(19) United States
(12) Patent Application Publication
(10) Pub. No.: US 2024/0173421 A1
(43) Pub. Date: May 30, 2024

(54) MODULATION OF ANTIBODY-DEPENDENT CELLULAR CYTOTOXICITY

(71) Applicant: Seagen Inc., Bothell, WA (US)

(72) Inventors: Philip MOQUIST, Seattle, WA (US); Matthew R. LEVENGOOD, Bothell, WA (US); Christopher I. LEISKE, Kirkland, WA (US); Noah A. BINDMAN, Seattle, WA (US)

(21) Appl. No.: 18/486,027

(22) Filed: Oct. 12, 2023

Related U.S. Application Data
(63) Continuation of application No. PCT/US2022/025610, filed on Apr. 20, 2022.
(60) Provisional application No. 63/177,218, filed on Apr. 20, 2021.

(51) Int. Cl. A61K 47/60; A61K 47/68; A61P 35/00; C07K 16/28

(52) U.S. Cl. CPC A61K 47/60; A61K 47/6849; A61K 47/6889; A61P 35/00; C07K 16/2878; C07K 2317/24; C07K 2317/72; C07K 2317/732

(57) ABSTRACT

The present disclosure provides, inter alia, antibodies with covalently attached biocompatible polymeric moieties, that are useful in treating various diseases such as cancer and autoimmune disorders.

### CROSS-REFERENCE

This application claims the benefit of U.S. Provisional Application No. 63/177,218, filed Apr. 20, 2021, which application is incorporated herein by reference.

### SEQUENCE LISTING

The instant application contains a Sequence Listing which has been submitted electronically in ASCII format and is hereby incorporated by reference in its entirety. Said ASCII copy, created on Apr. 19, 2022, is named 114093-718743_SL.txt and is 481,258 bytes in size.

### BACKGROUND

Antibody-dependent cellular cytotoxicity (ADCC) is an Fc-dependent effector function important for efficacious antibody therapy. ADCC is an immune reaction where antibodies bind a target cell or microbe; immune cells then bind the antibodies and release substances that lyse the target cell or microbe. See, e.g., Wang et al., Prot. Cell 2018: 9; 67-73. Natural killer (NK)-cell mediated ADCC is primarily triggered by IgG-subclasses IgG1 and IgG3 through the IgG-Fc-receptor (Fc gamma receptor) Ma. Binding of the Fc receptor induces release of granzymes, which induce apoptosis, and perforins, which oligomerize and form pores in the membranes of target cells. See, e.g., Trapani and Smyth, Nat. Rev. Immunol. 2002: 2; 735-47 and Smyth, et al., Mol. Immunol. 2005: 42; 501-10. However, while ADCC is an important mechanism of action for monoclonal antibody therapies, off-target antibody binding or immune cell activation can result in undesired side effects such as infusion-related reactions (IRRs) and systemic cytokine release syndrome (CRS). See, e.g., Tawara, et al., J. Immunol. 2008; 180:2294-8 and Wang, et al., Front. Immunol. 2015: 6; 368. Indeed, many of the most frequently administered cancer immunotherapies are associated with IRRs. See Caceres, et al., Ther. Clin. Risk Manag. 2019: 15; 965-977. In addition, antibodies with enhanced Fc receptor (FcR) binding affinity due to afucosylation or genetic engineering in the Fc region of the antibody are expected to be more prone to exhibit these undesired side effects. Thus, there remains a need for modulating the effector function of therapeutic antibodies to maintain potency and favorable pharmacokinetics while reducing off-target effects, such as these resulting from systemic Fc gamma receptor IIIa binding.

### SUMMARY

Aspects of the present disclosure provide a modulated effector function (MEF) antibody, wherein the MEF antibody comprises an effector function enhancing modification and an effector function diminishing modification, wherein the effector function diminishing modification comprises a biocompatible polymeric moiety (BPM) with a covalent attachment to an amino acid or post-translational modification of the MEF antibody. In some embodiments, the effector function diminishing modification is at least partially reversible. In some embodiments, the covalent attachment is cleavable, cleavage of which covalent attachment at least partially reverses the effector function diminishing modification. In some embodiments, the BPM comprises a cleavable moiety separate from the covalent attachment, cleavage of which moiety at least partially reverses the effector function diminishing modification.

In some embodiments, the effector function enhancing modification increases a binding affinity of the MEF antibody for FcγRI, FcγRIIa, FcγRIIb, FcγRIIIa, FcγRIIIb, or a combination thereof. In some embodiments, the effector function enhancing modification comprises afucosylation, a bisecting N-acetyl glucosamine, an S298A Fc region mutation, an E333A Fc region mutation, a K334A Fc region mutation, an S239D Fc region mutation, an I332E Fc region mutation, a G236A Fc region mutation, an S239E Fc region mutation, an A330L Fc region mutation, a G236A Fc region mutation, a L234Y Fc region mutation, a G236W Fc region mutation, an S296A Fc region mutation, an F243 Fc region mutation, an R292P Fc region mutation, a Y300L Fc region mutation, a V305L Fc region mutation, a P396L Fc region mutation, or a combination thereof. In some embodiments, the effector function enhancing modification comprises afucosylation.

In some embodiments, the amino acid comprises a cysteine residue or a methionine residue. In some embodiments, the covalent attachment to the cysteine residue comprises a disulfide bond, a thioether bond, a thioallyl bond, a vinyl thiol bond, or a combination thereof. In some embodiments, the disulfide bond, the thioallyl bond, or the combination thereof is cleavable. In some embodiments, the methionine residue couples to the BPM through a sulfanimine. In some embodiments, the post-translational modification comprises glycosylation, nitrosylation, phosphorylation, citrullination, sulfenylation, or a combination thereof.

In some embodiments, the BPM comprises an enzymatically cleavable moiety. In some embodiments, the enzymatically cleavable moiety comprises a protease cleavage sequence, a glycosidic group, a carbamate, a urea, a quaternary ammonium, or a combination thereof. In some embodiments, the enzymatically cleavable moiety comprises a protease cleavage sequence. In some embodiments, the protease cleavage sequence is a tumor-associated protease cleavage sequence. In some embodiments, the protease cleavage sequence is a cleavage sequence of thrombin, cathepsin, a matrix metalloproteinase, PAR-1 activating peptide, kallikrein, granzyme, caspase, ADAM, calpain, prostate-specific antigen, fibroblast activation protein, dipeptidyl peptidase IV, or a combination thereof.

In some embodiments, the effector function diminishing modification is at least partially reversible. In some embodiments, prior to the at least partial reversal of the effector function diminishing modification, the MEF antibody has between 2% and 20% of an effector function activity of an equivalent antibody lacking the BPM. In some embodiments, the MEF antibody has between 30% and 70% of the effector function activity of an equivalent antibody lacking the BPM following 192 hours incubation in 37° C. human plasma. In some embodiments, prior to the at least partial reversal of the effector function diminishing modification, the MEF antibody has between 2% and 20% of an FcγRIII binding affinity of an equivalent antibody lacking the BPM. In some embodiments, 192 hours after administration, the MEF antibody has between 30% and 70% of an FcγRIII binding affinity of an equivalent antibody lacking the BPM. In some embodiments, a rate of clearance of the MEF antibody is between 25% and 200% of a rate of the at least partial reversal of the effector function diminishing modification.

Aspects of the present disclosure provide a modulated effector function (MEF) antibody coupled to a plurality of biocompatible polymeric moieties (BPM) and an Fc which is at least partially blocked by the BPM, or a combination thereof; wherein a BPM of the plurality of BPMs is attached to a sulfur atom of a cysteine residue by a cleavable moiety comprising a disulfide bond.

Aspects of the present disclosure provide a modulated effector function (MEF) antibody coupled to a plurality of biocompatible polymeric moieties (BPM) and an Fc which is at least partially blocked by the BPM; wherein a BPM of the plurality of BPMs is attached to a methionine residue by a cleavable moiety.

Aspects of the present disclosure provide a modulated effector function (MEF) antibody comprising at least one Fc region and coupled to a plurality of biocompatible polymeric moieties (BPM) comprising cleavable moieties and present in a ratio of between 6 and 10 to Fc regions of the at least one Fc region; wherein the plurality of biocompatible polymeric moieties comprise molecular weights of between 500 and 2500 Daltons (Da); and wherein the cleavable moieties comprise cleavage rates of between 0.1 and 0.5 day⁻¹ in 37° C. human plasma.

Aspects of the present disclosure provide a modulated effector function (MEF) antibody, wherein the MEF antibody has 1, 2, 3, or 4 reduced interchain disulfide bonds and 2, 4, 6, or 8 biocompatible polymeric moieties (BPMs), respectively; wherein each BPM is covalently attached to a sulfur atom of a cysteine residue of a reduced interchain disulfide bond of the MEF antibody via a cleavable moiety; and wherein the MEF antibody exhibits time-dependent reduction in FcR binding, and thus a corresponding time-dependent reduction in an effector function, relative to that of an equivalent antibody.

In some embodiments, the MEF antibody has between 2% and 20% of the effector function activity of an equivalent antibody lacking the BPM. In some embodiments, the MEF antibody has between 2% and 10% of the effector function activity of an equivalent antibody lacking the BPM. In some embodiments, the MEF antibody has between 30% and 70% of the effector function activity of an equivalent antibody lacking the BPM following 192 hours incubation in 37° C. human plasma. In some embodiments, the MEF antibody has less than 50% of the effector function activity of an equivalent antibody lacking the BPM following cleavage of half of its BPMs. In some embodiments, the cleavable moiety comprises a cleavage rate of between 100% and 500% of its physiological clearance rate during in vivo circulation in an adult human male. In some embodiments, the cleavable moiety comprises a cleavage rate of between 50% and 300% of its physiological clearance rate during in vivo circulation in an adult human male.

In some embodiments, the cleavable moiety is configured to undergo a secondary reaction which diminishes a rate of its cleavage. In some embodiments, the cleavable moiety comprises a succinimide, and wherein the secondary reaction comprises succinimide hydrolysis. In some embodiments, the cleavable moiety is configured to undergo the BPM cleavage at least at twice the rate of the secondary reaction during in vivo circulation in an adult human male.

In some embodiments, each cleavable moiety is covalently attached to a sulfur atom of a cysteine residue of a reduced interchain disulfide bond of the MEF antibody through a cleavable disulfide bond, or through a cleavable thioether bond to a non-hydrolyzed succinimide moiety. In some embodiments, the non-hydrolyzed succinimide is configured to undergo thioether cleavage faster than hydrolysis in 37° C. human plasma. In some embodiments, each cleavable moiety is covalently attached to a sulfur atom of a cysteine residue of a reduced interchain disulfide bond of the MEF antibody through a thioether bond to a hydrolyzed succinimide moiety. In some embodiments, each cleavable moiety is covalently attached to a sulfur atom of a cysteine residue of a reduced interchain disulfide bond of the MEF antibody through the cleavable disulfide bond.

In some embodiments, each cleavable moiety comprises a structure according either to Formula (II) or (III):

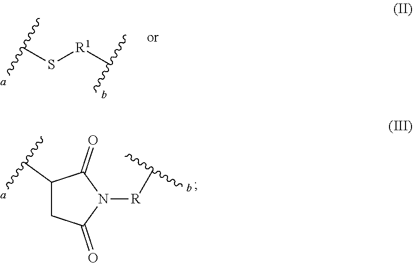

wherein:
R¹ is a C₂-C₁₂ alkylene, optionally interrupted with one of —NH—C(═O)—, —C(═O)NH—, —NH—, and —O—;
R is absent, or is a C₁-C₁₂ alkylene optionally interrupted with one or two of phenyl, —NH—C(═O)—, —C(═O)NH—, —NH—, —O—, —O—C(═O)—, —C(═O)O—, —S—C(═O)—, —C(═O)S—, —O—C(═O)O—, —C(═NR^{1A}), an acetal, —O(SO₂)O—, —O—[P(═O)(—OH)]O—, —C(═N—OH)—, —C(═N—NH₂)—, and —C(R^{1A})═N—NH—; and R is optionally substituted with 1-3 substituents independently selected from phenyl, oxo, and —CO₂R^{A}; C₃-C₆ cycloalkylene; and phenyl optionally substituted with 1-3 independently selected C₁-C₃ alkoxy;
each R^{A} is independently hydrogen or C₁-C₆ alkyl;
each R^{1A} is independently hydrogen or C₁-C₆ alkyl;
wherein [symbol] (a) represents the covalent attachment to the cysteine residue of the reduced interchain disulfide bond of the MEF antibody; and [symbol]
   (b) represents the covalent attachment to a BPM or the remainder of the cleavable moiety, which retains covalent attachment to a BPM.

In some embodiments, each cleavable moiety has a structure according to Formula (III):

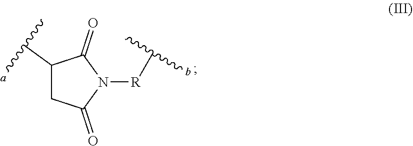

and
wherein R is a C₁-C₁₂ alkylene interrupted with —C(═N—NH₂)— or —C(R^{1A})═N—NH—; or interrupted with phenyl and one of —C(═N—NH₂)— and —C(R^{1A})═N—NH—.

In some embodiments, each cleavable moiety comprises a structure of any one of Formulas (IIa)-(IIi):

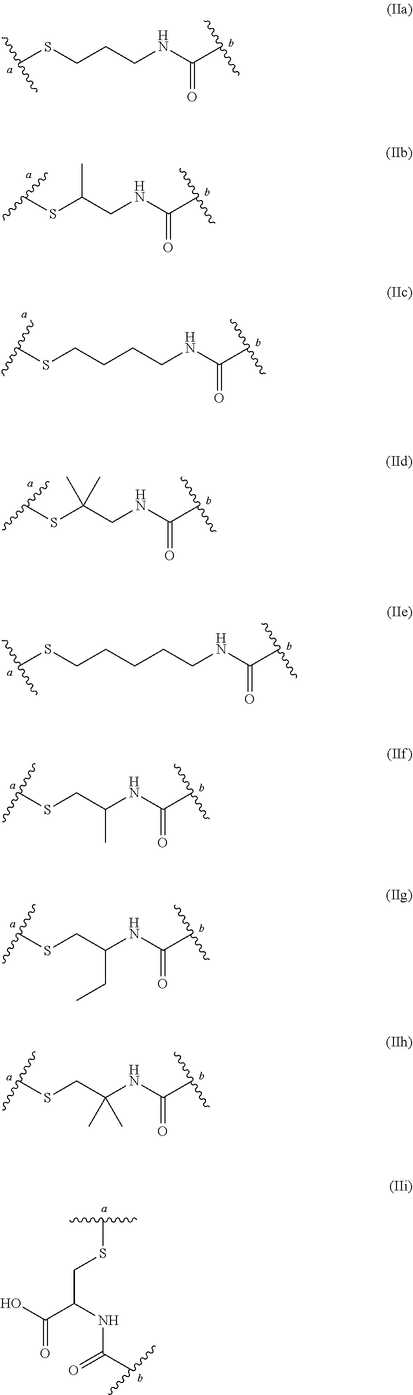

wherein [symbol] (a) represents the covalent attachment to the sulfur atom of a cysteine residue of a reduced interchain disulfide bond of the MEF antibody; and [symbol] (b) represents the covalent attachment of the cleavable moiety to a BPM.

In some embodiments, each cleavable moiety comprises a structure according to Formula (IIIl):

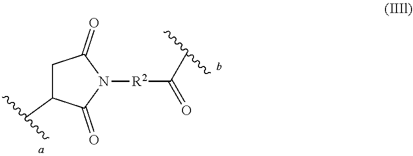

wherein:
R² is C₁-C₁₅ alkyl optionally substituted with one or more instances of hydroxyl, halogen, —CN, C₁-C₆ alkyl, C₁-C₆ alkenyl, C₁-C₆ alkynyl, C₁-C₆ alkoxyl, C₁-C₆ thioalkoxy, —C₁-C₆ cycloalkyl, —NR³R⁴, —C(═O)—R³, —C(═O)—OR⁵, PEG2-PEG72, or a combination thereof;
R³ and R⁴ are each independently selected from the group consisting of H,
[symbol] (a) represents the covalent attachment to the sulfur atom of a cysteine residue of a reduced interchain disulfide bond of the MEF antibody; and [symbol]
   (b) represents the covalent attachment of the cleavable moiety to a BPM.

In some embodiments, R² is C₁-C₁₅ alkyl optionally substituted with one or more instances of hydroxyl, halogen, —CN, C₁-C₆ alkyl, C₁-C₆ alkoxyl, or a combination thereof. In some embodiments, R² is C₁-C₁₂ alkyl optionally substituted with one or more instances of hydroxyl, halogen, —CN, C₁-C₃ alkyl, C₁-C₃ alkoxyl, or a combination thereof. In some embodiments, R² is C₁-C₁₂ alkyl optionally substituted with one or more instances of hydroxyl, halogen, or C₁-C₃ alkyl.

In some embodiments, each cleavable moiety comprises a structure of any one of Formulas (IIIh)-(IIIk):

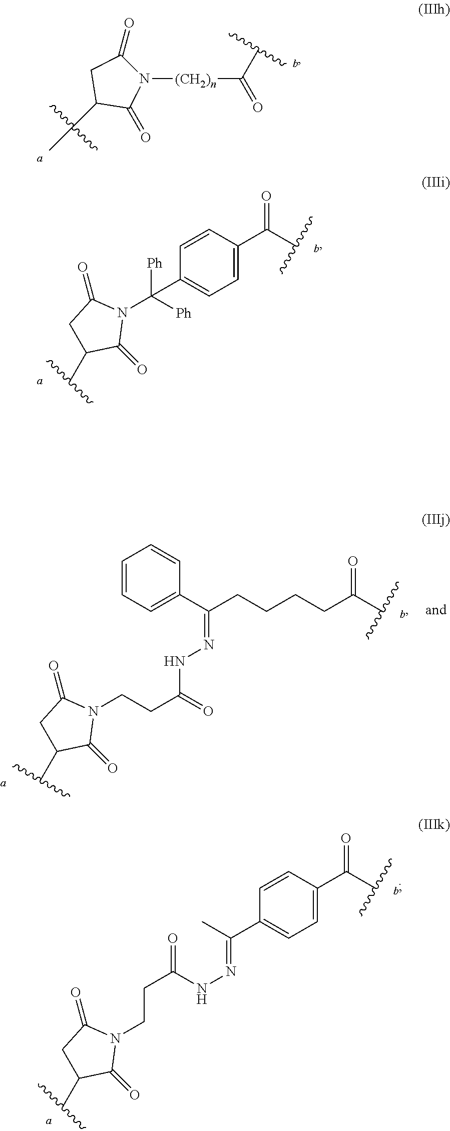

wherein subscript n is an integer ranging from 2 to 8; and
   wherein [symbol] (a) represents the covalent attachment to the sulfur atom of a cysteine residue of a reduced interchain disulfide bond of the MEF antibody; and [symbol] (b) represents the covalent attachment of the cleavable moiety to a BPM.

In some embodiments, each cleavable moiety comprises a structure according to Formula (IIIh):

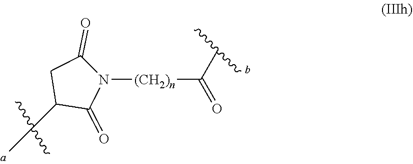

wherein subscript n is an integer ranging from 2 to 8; and
   wherein [symbol] (a) represents the covalent attachment to the sulfur atom of a cysteine residue of a reduced interchain disulfide bond of the MEF antibody; and [symbol] (b) represents the covalent attachment of the cleavable moiety to a BPM.

In some embodiments, the time-dependent reduction in FcR binding of the MEF antibody is characterized by an initial reduction in the binding of FcR from at least about 50% to about 90% relative to the equivalent antibody. In some embodiments, the initial reduction in FcR binding of the MEF antibody is characterized by a K_{D} that is about 2-fold to about 1,000-fold higher than the equivalent antibody. In some embodiments, the initial reduction of FcR binding is followed by a recovery of the binding as a further characteristic of the time-dependent reduction in FcR binding, wherein the recovery is correlated with BPM loss through non-enzymatic cleavage of the corresponding cleavable moiety(ies) in physiological media. In some embodiments, the physiological media is vertebrate plasma. In some embodiments, each of the cleavable moieties has a plasma half-life of from about 3 hours to about 96 hours. In some embodiments, the recovery substantially restores the FcR binding to that of the equivalent antibody after from about 3 hours to about 96 hours in vitro.

In some embodiments, the MEF antibody is fucosylated. In some embodiments, the MEF antibody is afucosylated. In some embodiments, the antibody of the MEF antibody is a therapeutic antibody.

In some embodiments, each BPM is a polyethylene glycol moiety, a polyketal moiety, a polyglycerol moiety, a polysaccharide moiety, a polysarcosine moiety, a polypeptide moiety, or a polyzwitterionic moiety. In some embodiments, each BPM is a monodispersed moiety. In some embodiments, each BPM comprises a monodispersed polyethylene glycol, polyglycerol, polypeptide, or polysaccharide moiety. In some embodiments, each BPM is a polydispersed moiety. In some embodiments, each BPM comprises a polydispersed polyethylene glycol, polyglycerol, polypeptide, or polysaccharide moiety. In some embodiments, each BPM independently has a weight-average molecular weight of about 100 Daltons to about 5,000 Daltons. In some embodiments, each BPM independently has a weight-average molecular weight of about 1,000 Daltons to about 3,000 Daltons. In some embodiments, each BPM independently has a hydrodynamic diameter of about 5 nm to about 25 nm. In some embodiments, each BPM independently has a hydrodynamic diameter of about 15 nm to about 25 nm. In some embodiments, each BPM independently has a hydrodynamic diameter of about 10 nm to about 20 nm. In some embodiments, each BPM independently has a hydrodynamic diameter of about 5 nm to about 15 nm. In some embodiments, each BPM independently has a hydrodynamic diameter of about 5 nm to about 10 nm. In some embodiments, each BPM comprises a monodispersed PEG2 to PEG72 moiety. In some embodiments, each BPM comprises a monodispersed PEG8 to PEG48 moiety. In some embodiments, each BPM comprises a monodispersed PEG12 to PEG24 moiety. In some embodiments, each BPM comprises a monodispersed branched PEG20 to PEG76 moiety; and wherein each branch comprises at least two contiguous ethylene glycol subunits. In some embodiments, each monodispersed branched PEG20 to PEG76 moiety has 2 to 8 branches. In some embodiments, each monodispersed branched PEG20 to PEG76 moiety has 2 to 6 branches. In some embodiments, each monodispersed branched PEG20 to PEG76 moiety has 2 to 4 branches. In some embodiments, each BPM is a PEG4(PEG8)₃ or a PEG4(PEG24)₃ moiety. In some embodiments, each polyethylene glycol moiety of a BPM has a cap selected from the group consisting of —CH₃, —CH₂CH₂CO₂H, and —CH₂CH₂NH₂. In some embodiments, each BPM has a structure selected from the group consisting of:

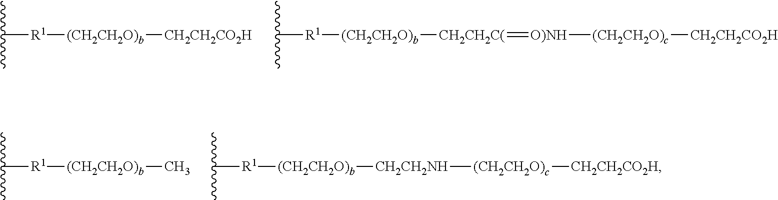

wherein R¹ is a C₂-C₁₂ alkylene, optionally interrupted with one of —NH—C(═O)—, —C(═O)NH—, —NH—, or —O— to which the cleavable moiety is covalently attached, and optionally substituted with —CO₂H;
each subscript b ranges from 2 to 72;
each subscript c ranges from 1 to 72; and [symbol]
   indicates site of covalent attachment to the cleavable moiety. In some embodiments, subscript b ranges from 6 to 72 and subscript c ranges from 1-12. In some embodiments, subscript b ranges from 8 to 72 and subscript c ranges from 1-12. In some embodiments, subscript b ranges from 10 to 72 and subscript c ranges from 1-12. In some embodiments, subscript b ranges from 12 to 72 and subscript c ranges from 1-12.

In some embodiments, each BPM and cleavable moiety, together with a sulfur atom of a cysteine residue of a reduced interchain disulfide bond of the MEF antibody to which the cleavable moiety is covalently attached, has a structure according to any one of Formulas (IIj-IIn):

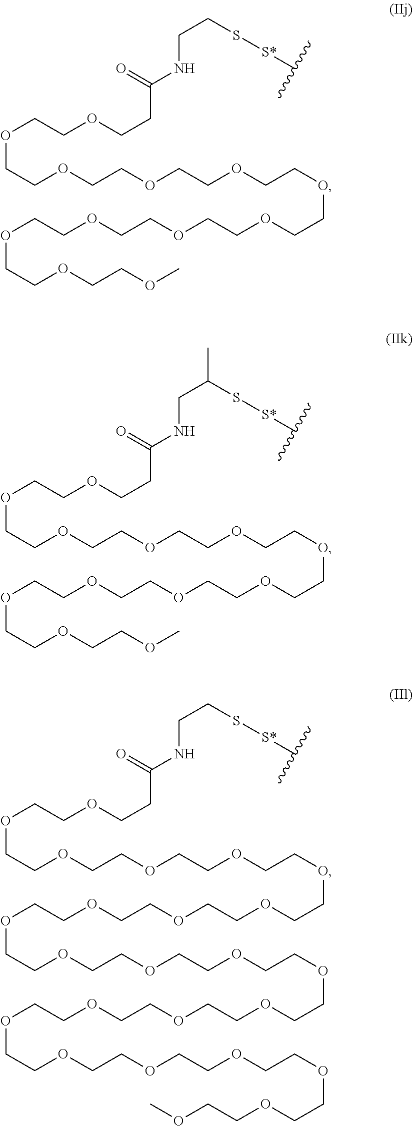

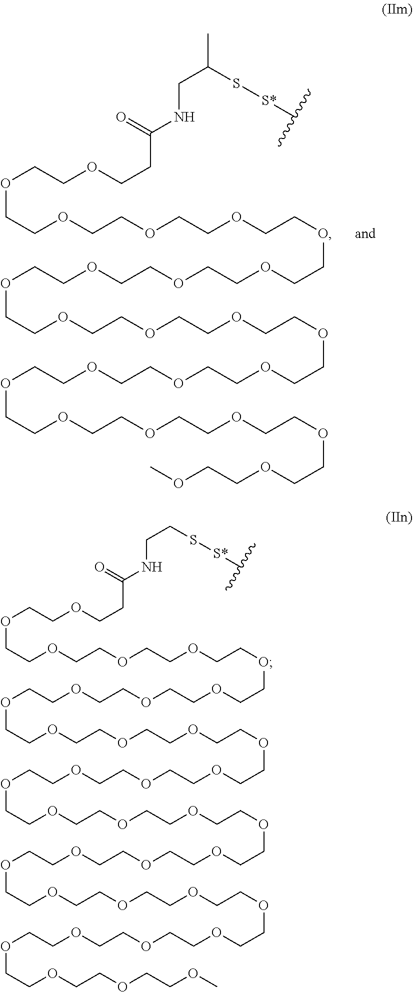

wherein S* is a sulfur atom from a cysteine residue of a reduced interchain disulfide bond of the MEF antibody; and
wherein [symbol] indicates covalent attachment to the remainder of the MEF antibody.

In some embodiments, each BPM and cleavable moiety, together with the sulfur atom of a cysteine residue of a reduced interchain disulfide bond of the MEF antibody, has a structure according to any one of Formulas (IIIa)-(IIIg):

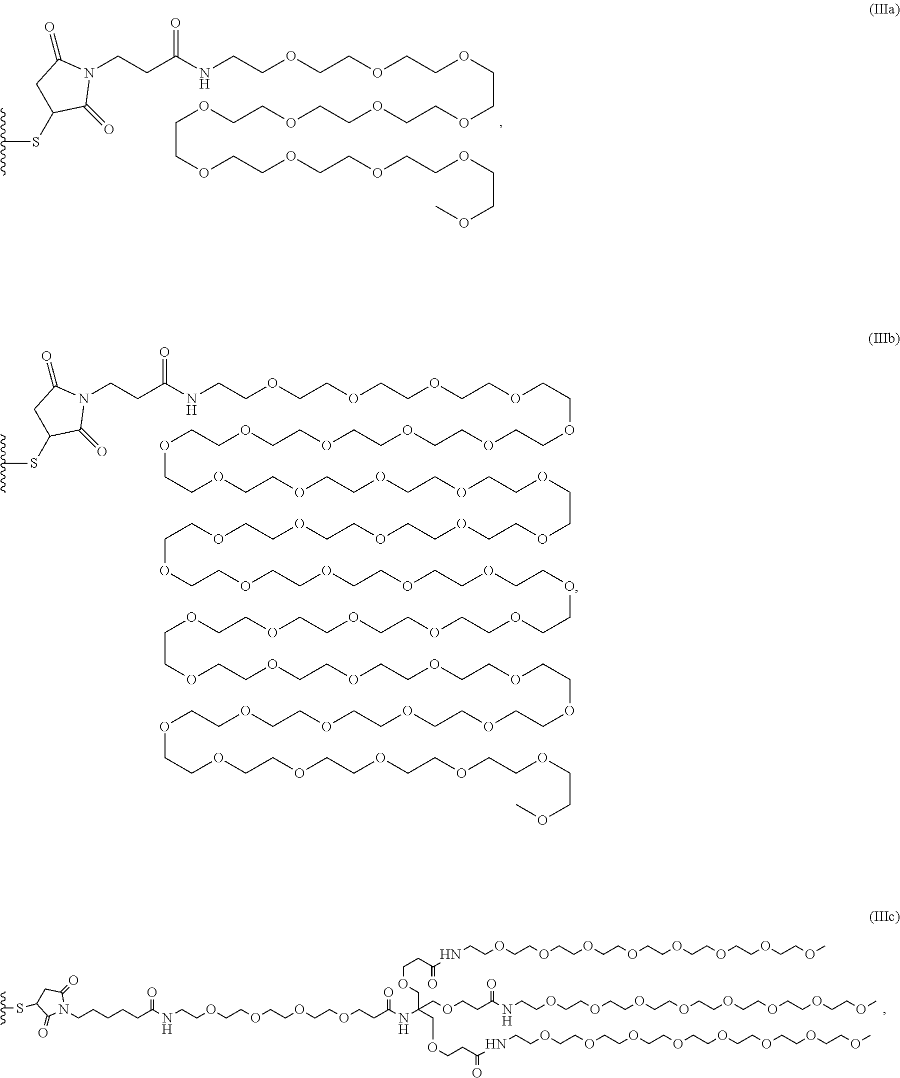 [SMILES: *SC1CC(=O)N(CCC(=O)NCCOCCOCCOCCOCCOCCOCCOCCOCCOCCOCCOCCOC)C1=O ; *SC1CC(=O)N(CCC(=O)NCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOC)C1=O ; *SC1CC(=O)N(CCCCCC(=O)NCCOCCOCCOCCOCCC(=O)NC(COCCC(=O)NCCOCCOCCOCCOCCOCCOCCOCCOC)(COCCC(=O)NCCOCCOCCOCCOCCOCCOCCOCCOC)COCCC(=O)NCCOCCOCCOCCOCCOCCOCCOCCOC)C1=O]

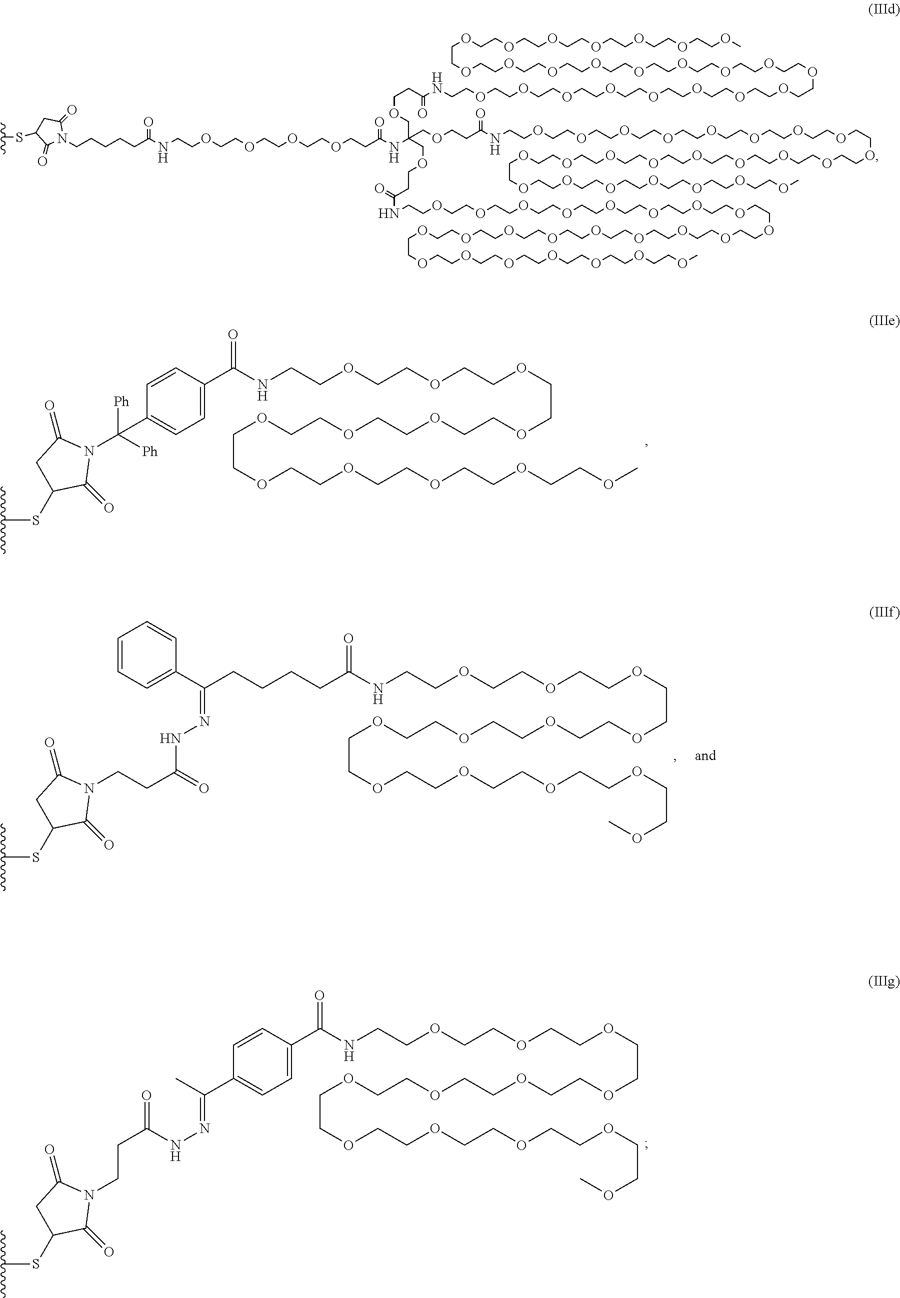

wherein [symbol] indicates covalent attachment to the remainder of the cysteine residue of the reduced interchain disulfide bond of the MEF antibody.

In some embodiments, the Fc receptor is present on a peripheral blood mononuclear cell (PBMC). In some embodiments, the Fc receptor is the Fc gamma Ma receptor. In some embodiments, the PBMC is a natural killer cell. In some embodiments, the PBMC is enriched from plasma of a normal donor. In some embodiments, the normal donor is a human having the Fc gamma receptor III 158 V/V genotype. In some embodiments, reduction in Fc receptor binding is determined by competitive binding of the MEF antibody and a labeled isotype matched IgG Fc fragment to an orthogonally labeled Fc receptor. In some embodiments, the IgG Fc fragment is the labeled isotype matched Fc domain of a human IgG₁ antibody. In some embodiments, the label of the isotype matched IgG Fc fragment comprises a fluorophore. In some embodiments, the labeled isotype matched IgG Fc fragment is immobilized on a solid support. In some embodiments, the orthogonal label of the Fc receptor comprises biotin. In some embodiments, the isoform of the Fc receptor is Fc gamma Ma or gamma Mb. In some embodiments, the effector function that is reduced relative to the equivalent antibody on administration of the MEF antibody to a subject is antibody-dependent cellular cytotoxicity (ADCC) or antibody-dependent cellular phagocytosis (ADCP). In some embodiments, the subject to whom the MEF antibody is administered is a human. In some embodiments, the MEF antibody is an IgG₁ antibody. In some embodiments, the MEF antibody is a monoclonal antibody. In some embodiments, the monoclonal antibody is a chimeric antibody. In some embodiments, the monoclonal antibody is a humanized antibody.

In some embodiments, the MEF antibody has one or more mutations in the Fc region; wherein the MEF antibody having the one or more mutations has higher effector function relative to the equivalent antibody. In some embodiments, the MEF antibody is an IgG₁ antibody; and the one or more mutations in the Fc region are selected from the group consisting of S298A, E333A, K334A, S239D, I332E, G236A, S239E, A330L, I332E, G236A, S239D, I332E, G236A, L234Y, G236W, S296A, F243, R292P, Y300L, V305L, and P396L.

In some embodiments, the MEF antibody binds to a cancer cell. In some embodiments, the MEF antibody binds to an immune cell. In some embodiments, the MEF antibody binds to human CD40. In some embodiments, the antibody comprises rituximab, obinutuzumab, ofatumumab, trastuzumab, alemtuzumab, mogamulizumab, cetuximab, or dinutuximab. In some embodiments, the MEF antibody comprises a sequence which has at least 80% sequence identity to SEQ ID NO: 890. In some embodiments, the MEF antibody comprises a sequence which has at least 80% sequence identity to SEQ ID NO: 891. In some embodiments, the MEF antibody comprises a sequence which has at least 80% sequence identity to the heavy chain variable region of SEQ ID NO: 890. In some embodiments, the MEF antibody comprises a sequence which has at least 80% sequence identity to the light chain variable region of SEQ ID NO: 891. In some embodiments, the MEF antibody has a dissociation constant of at most 500 nM for the human CD40. In some embodiments, the MEF antibody has a dissociation constant of at most 10 nM for the human CD40.

In some embodiments, when the MEF antibody is introduced to a population of cells comprising one or more target cells, the binding of the MEF antibody to the one or more target cells provides a time-dependent reduction in peripheral cytokine levels relative to peripheral cytokine levels provided by binding of an equimolar amount of the equivalent antibody. In some embodiments, the time-dependent reduction of peripheral cytokine levels is characterized by an initial reduction of at least about 50%. In some embodiments, the time-dependent reduction of peripheral cytokine levels is characterized by an initial reduction of at least about 80%. In some embodiments, the time-dependent reduction of peripheral cytokine levels is characterized by recovery of the peripheral cytokine levels to at least about 50% relative to that from an equimolar amount of the equivalent antibody after from about 48 h to about 96 h. In some embodiments, the time-dependent reduction of peripheral cytokine levels is characterized by recovery of the peripheral cytokine levels to about 100% relative to that from an equimolar amount of the equivalent antibody after from about 48 h to about 96 h. In some embodiments, the population of cells is a biological sample; and wherein the time-dependent reduction in peripheral cytokine levels is characterized by an initial reduction in peripheral cytokine levels in the supernatant of the biological sample relative to that from an equimolar amount of the equivalent antibody. In some embodiments, the population of cells is in a subject; and wherein the peripheral cytokine levels are systemic cytokine levels in the plasma of the subject. In some embodiments, when the MEF antibody is introduced to a population of cells comprising one or more target cells, the binding of the MEF antibody to the one or more target cells provides an initial reduction in the rate of cell lysis of the one or more target cells relative to the rate of cell lysis provided by binding of an equimolar amount of the equivalent antibody. In some embodiments, the population of cells is a biological sample. In some embodiments, the population of cells is in a subject. In some embodiments, the one or more target cells comprise cancer cells comprising antigens or immune cells comprising antigens. In some embodiments, the target cells are radiolabeled. In some embodiments, the population of cells further comprises normal PBMCs. In some embodiments, the normal PBMCs comprise natural killer cells.

In some embodiments, administration of the MEF antibody to a subject provides a reduction of about 20% to about 75% in cytokine Cₘₐₓ relative to administration of an equimolar amount of the equivalent antibody. In some embodiments, administration of the MEF antibody to a subject provides substantially the same total antibody AUC_{0-∞}. relative to administration of an equimolar amount of the equivalent antibody.

Aspects of the present disclosure provide a composition comprising a distribution of MEF antibodies as disclosed herein. In some embodiments, the composition comprises a unit dose of the distribution of MEF antibodies. In some embodiments, the unit dose does not increase systemic levels of monocyte chemotactic protein-1 (MCP-1), tumor necrosis factor (TNF-α), interferon gamma (IFN-γ), interleukin 1 beta (IL1B), interleukin 6 (IL6), or interleukin 10 (IL10) of more than 10-fold above levels prior to the administering. In some embodiments, the composition further comprises at least one pharmaceutically acceptable carrier.

Aspects of the present disclosure provide a composition comprising a first population of MEF antibodies; a second population of MEF antibodies; and at least one pharmaceutically acceptable carrier; wherein the BPMs present in the first population of MEF antibodies are different than the BPMs present in the second population of MEF antibodies.

Aspects of the present disclosure provide a composition comprising a first population of MEF antibodies; a second population of MEF antibodies; and at least one pharmaceutically acceptable carrier; wherein the cleavable moieties present in the first population of MEF antibodies are different than the cleavable moieties present in the second population of MEF antibodies.

In some embodiments, the sole active ingredient in the composition is the MEF antibody. In some embodiments, the percent aggregation of the MEF antibody in the composition is increased by about 1-fold to about 1.1 fold relative to an equivalent antibody. In some embodiments, at least 90% of antibodies of a distribution of MEF antibodies are afucosylated. In some embodiments, at least 90% of antibodies of a plurality of MEF antibodies are afucosylated. In some embodiments, at least 98% of antibodies of a distribution of MEF antibodies are afucosylated. In some embodiments, at least 98% of antibodies of a plurality of MEF antibodies are afucosylated.

Aspects of the present disclosure provide a method of treating a condition in a subject in need thereof, the method comprising: administering to the subject a therapeutically effective amount of a composition comprising a modulated effector function (MEF) antibody which comprises an effector function diminishing modification, and which effector function diminishing modification is at least partially reversible under physiological conditions; and treating the condition while maintaining a systemic level of a cytokine or an inflammatory marker to no more than 10-fold above a level prior to the administering.

In some embodiments, the cytokine or the inflammatory marker is monocyte chemotactic protein-1 (MCP-1), macrophage inflammatory protein-1 (MIP-1β), tumor necrosis factor (TNF-α), interferon gamma (IFN-γ), interleukin-1 receptor agonist (IL-1RA), interleukin 1 beta (IL1B), interleukin 6 (IL6), interleukin 10 (IL10), or a combination thereof. In some embodiments, the cytokine or the inflammatory marker is monocyte chemotactic protein-1 (MCP-1), macrophage inflammatory protein-1 (MIP-1β), interleukin-1 receptor agonist (IL-1RA), or a combination thereof. In some embodiments, the modification comprises a cleavable biocompatible polymeric moiety (BPM) covalently attached to an amino acid residue or a post-translational modification of the MEF antibody. In some embodiments, prior to the BPM cleavage, the MEF antibody has between 2% and 20% of the effector function activity of an equivalent antibody lacking the BPM. In some embodiments, 192 hours after administration, the MEF antibody has between 30% and 70% of the effector function activity of an equivalent antibody lacking the BPM. In some embodiments, a rate of clearance of the MEF antibody is between 25% and 200% of a rate cleavage of the BPM. In some embodiments, the modification which decreases the effector function of the MEF antibody decreases FcγRIII binding affinity of the MEF antibody.

Aspects of the present disclosure provide a method of decreasing the severity of an infusion related reaction in a subject associated with an antibody, comprising intravenously administering to the subject a composition consistent with the present disclosure; wherein the antibody is equivalent to an MEF antibody of the composition; and wherein the severity of the infusion related reaction is decreased from 1 to 4 units relative to intravenous administration of an equimolar amount of the antibody.

Aspects of the present disclosure provide a method of reducing the incidence of and/or risk of developing an infusion related reaction in a subject associated with an antibody, comprising intravenously administering to the subject a composition comprising a composition consistent with the present disclosure; wherein the antibody is equivalent to an MEF antibody of the composition; and wherein the incidence of and/or risk of developing the infusion related reaction is reduced relative to intravenous administration of an equimolar amount of an equivalent antibody.

Aspects of the present disclosure provide a method of reducing one or more symptoms of an infusion related reaction in a subject associated with an antibody, comprising intravenously administering to the subject a composition comprising a composition consistent with the present disclosure; wherein the antibody is equivalent to an MEF antibody of the composition; wherein the one or more symptoms of the infusion related reaction are reduced relative to intravenous administration of an equimolar amount of an equivalent antibody.

Aspects of the present disclosure provide a method of decreasing the Cₘₐₓ of an active antibody, comprising intravenously administering to a subject a composition comprising a composition consistent with the present disclosure; wherein the active antibody is equivalent to an MEF antibody of the composition; and wherein the Cₘₐₓ of the active antibody after intravenous administration of the MEF antibody composition is decreased relative to the Cₘₐₓ after intravenous administration of an equimolar amount of the active antibody.

Aspects of the present disclosure provide a method of delaying maximal Fc gamma receptor IIIa binding of an antibody in a subject, comprising intravenously administering to the subject a composition comprising a composition consistent with the present disclosure; wherein the antibody is equivalent to an MEF antibody of the composition; and wherein the MEF antibody delays binding to Fc gamma receptor IIIa relative to the antibody.

Aspects of the present disclosure provide a method of selectively increasing binding of an antibody to Fc gamma receptor IIIa in a target cell in a subject, comprising intravenously administering to the subject a composition comprising a composition consistent with the present disclosure; wherein the antibody is equivalent to an MEF antibody of the composition; and wherein the ratio of the MEF antibody (i) bound to Fc gamma receptor IIIa at the target cell and (ii) bound to Fc gamma receptor IIIa systemically is increased relative to the ratio of the antibody (i) bound to Fc gamma receptor IIIa at the target cell and (ii) bound to Fc gamma receptor IIIa systemically.

Aspects of the present disclosure provide a method of reducing systemic Fc gamma receptor IIIa activation in a subject after administration of an antibody, comprising intravenously administering to the subject a composition comprising a composition consistent with the present disclosure; wherein the antibody is equivalent to an MEF antibody of the composition; and wherein the administration of the MEF antibody provides reduced systemic activation of Fc gamma receptor IIIa relative to intravenous administration of an equimolar amount of the antibody.

Aspects of the present disclosure provide a method of decreasing systemic cytokine production in a subject after administration of an antibody, comprising intravenously administering to the subject a composition comprising a composition consistent with the present disclosure; wherein the antibody is equivalent to an MEF antibody of the composition; and wherein administration of the composition comprising the MEF antibody decreases systemic cytokine production relative to intravenous administration of an equimolar amount of the antibody.

In some embodiments, each cleavable moiety comprises a structure according to Formula (II):

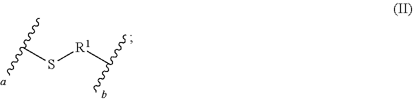

wherein at least about 10% of the BPMs are cleaved from the MEF antibody within about 12 hours and at least about 25% of the BPMs are cleaved from the MEF antibody within 48 hours after intravenous administration.

In some embodiments, each cleavable moiety comprises a structure according to Formula (III):

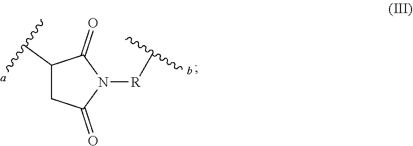

wherein about 10% of the BPMs are cleaved from the MEF antibody within about 12 hours and about 25% of the BPMs are cleaved from the MEF antibody within 48 hours after intravenous administration.

In some embodiments, at least about 10% of the BPMs are cleaved from the MEF antibody within about 12 hours and at least about 30% of the BPMs are cleaved from the MEF antibody within 48 hours after intravenous administration. In some embodiments, at least about 20% of the BPMs are cleaved from the MEF antibody within about 12 hours and at least about 40% of the BPMs are cleaved from the MEF antibody within 48 hours after intravenous administration. In some embodiments, at least about 30% of the BPMs are cleaved from the MEF antibody within about 12 hours and at least about 50% of the BPMs are cleaved from the MEF antibody within 48 hours after intravenous administration. In some embodiments, at least about 50% of the BPMs are cleaved from the MEF antibody within about 12 hours and about 100% of the BPMs are cleaved from the MEF antibody within 48 hours after intravenous administration. In some embodiments, at least about 50% of the BPMs are cleaved from the MEF antibody within about 12 hours.

In some embodiments, the MEF antibody is a therapeutic antibody. In some embodiments, the MEF antibody is selected from the group consisting of rituximab, obinutuzumab, ofatumumab, trastuzumab, alemtuzumab, mogamulizumab, cetuximab and dinutuximab.

Aspects of the present disclosure provide an MEF antibody having the structure:

Ab-(S*—X-BPM)ₚ
wherein:
each S* is a sulfur atom from a cysteine residue of a reduced interchain disulfide of the MEF antibody;
each X is a cleavable moiety; each BPM is a polyethylene glycol moiety, a polyketal moiety, a polyglycerol moiety, a polysaccharide moiety, a polysarcosine moiety, a polypeptide moiety, or a polyzwitterionic moiety;
subscript p is 2, 4, 6, or 8; and
   Ab represents the remainder of the antibody.

In some embodiments, each X is covalently attached to a sulfur atom of a cysteine residue of a reduced interchain disulfide bond of the MEF antibody through a cleavable disulfide bond, or through a cleavable thioether bond to a non-hydrolyzed succinimide moiety. In some embodiments, each X is covalently attached to a sulfur atom of a cysteine residue of a reduced interchain disulfide bond of the MEF antibody through the cleavable thioether bond. In some embodiments, each X is covalently attached to a sulfur atom of a cysteine residue of a reduced interchain disulfide bond of the MEF antibody through the cleavable disulfide bond.

In some embodiments, each X comprises a structure of either Formula (II) or (III):

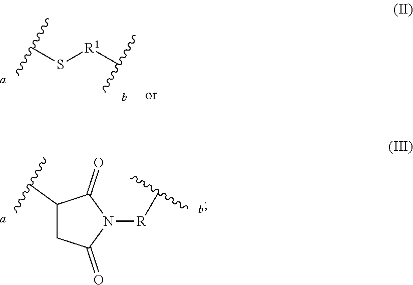

wherein:
R¹ is a C₂-C₁₂ alkylene, optionally interrupted with one of —NH—C(═O)—, —C(═O)NH—, —NH—, and —O—;
R is absent, or is a C₁-C₁₂ alkylene optionally interrupted with one or two of phenyl, —NH—C(═O)—, —C(═O)NH—, —NH—, —O—, —O—C(═O)—, —C(═O)O—, —S—C(═O)—, —C(═O)S—, —O—C(═O)O—, —C(═NR^{1A}), an acetal, —O(SO₂)O—, —O—[P(═O)(—OH)]O—, —C(═N—OH)—, —C(═N—NH₂)—, and —C(R^{1A})═N—NH—; and R is optionally substituted with 1-3 substituents independently selected from phenyl, oxo, and —CO₂R^{A}; C₃-C₆ cycloalkylene; and phenyl optionally substituted with 1-3 independently selected C₁-C₃ alkoxy;
each R^{A} is independently hydrogen or C₁-C₆ alkyl;
each R^{1A} is independently hydrogen or C₁-C₆ alkyl;
wherein [symbol] (a) represents the covalent attachment to the cysteine residue of the reduced interchain disulfide bond of the MEF antibody; and [symbol]
   (b) represents the covalent attachment to a BPM or the remainder of X, which retains covalent attachment to a BPM.

In some embodiments, each X comprises a structure according to Formula (III):

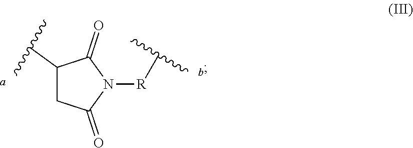

and
wherein R is a C₁-C₁₂ alkylene interrupted with —C(═N—NH₂)— or —C(R^{1A})═N—NH—; or interrupted with phenyl and one of —C(═N—NH₂)— and —C(R^{1A})═N—NH—.

In some embodiments, each X comprises a structure according to any one of Formulas (IIa)-(IIi):

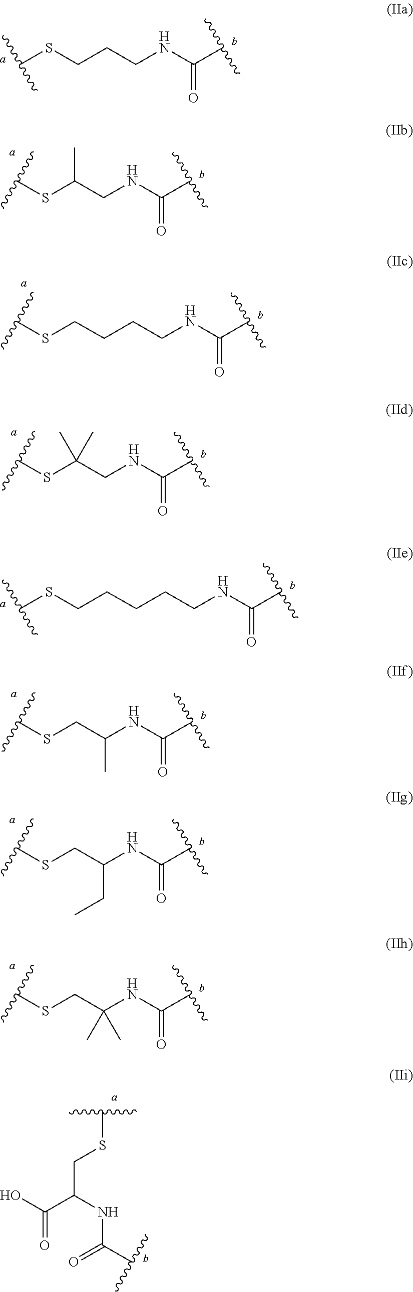

wherein [symbol] (a) represents the covalent attachment to the sulfur atom of a cysteine residue of a reduced interchain disulfide bond of the MEF antibody; and [symbol] (b) represents the covalent attachment of X to a BPM.

In some embodiments, each X comprises a structure according to any one of Formulas (IIIh)-(IIIk):

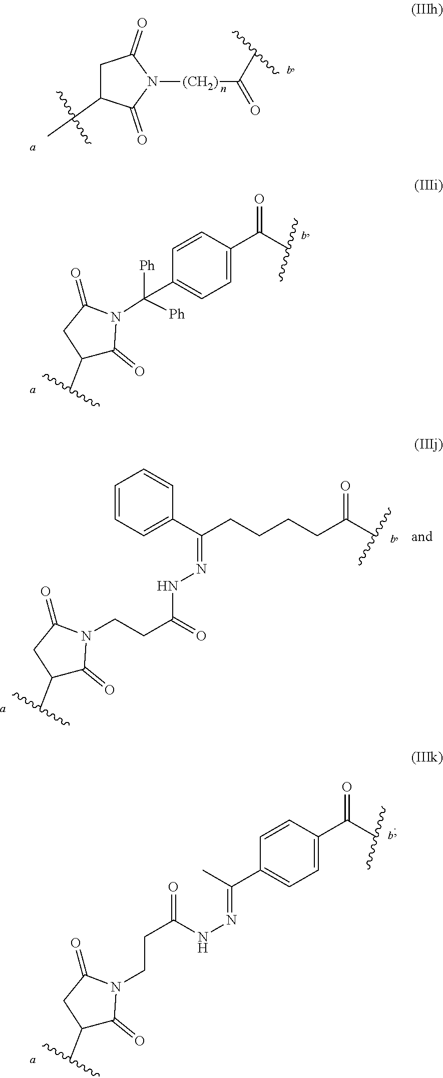

wherein subscript n is an integer ranging from 2 to 8; and
   wherein [symbol] (a) represents the covalent attachment to the sulfur atom of a cysteine residue of a reduced interchain disulfide bond of the MEF antibody; and [symbol] (b) represents the covalent attachment of X to a BPM.

In some embodiments, the MEF antibody has the structure of

Ab-(S*—X-BPM)ₚ
wherein each BPM moiety has a structure according to Formula (IVa):

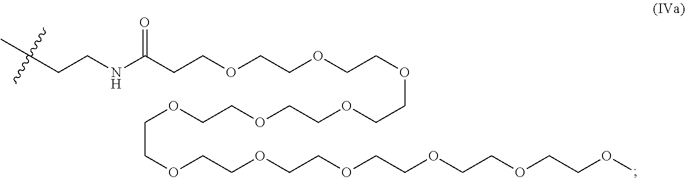

and
wherein [symbol] represents the covalent attachment to a cleavable moiety.

In some embodiments, the MEF antibody has the structure of:

Ab-(S*—X-BPM)ₚ
wherein each —X-BPM moiety has a structure according to Formula (Mb):

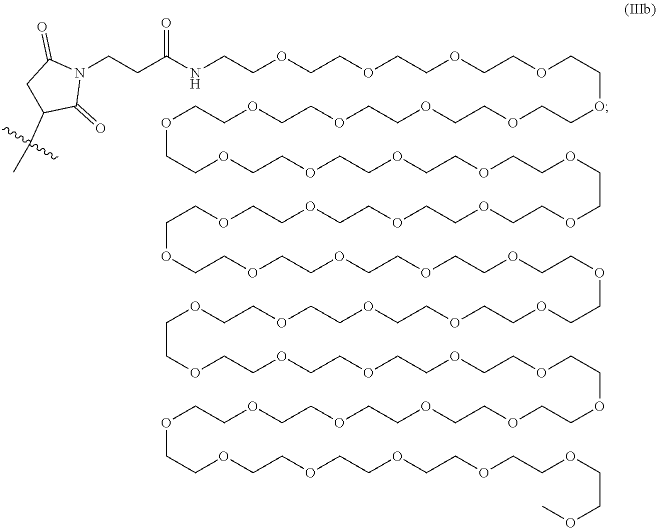 [SMILES: *C1CC(=O)N(CCC(=O)NCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOC)C1=O]

and
wherein [symbol] represents the covalent attachment to S*.

In some embodiments, the MEF antibody has the structure of:

Ab-(S*—X-BPM)ₚ
wherein each —X-BPM moiety has a structure according to formula (IIIm):

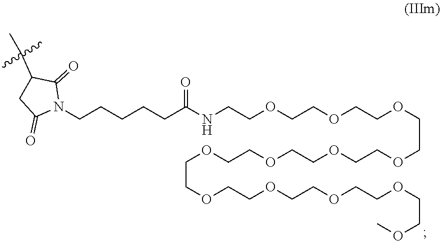 [SMILES: *C1CC(=O)N(CCCCCC(=O)NCCOCCOCCOCCOCCOCCOCCOCCOCCOCCOCCOCCOC)C1=O]

and
wherein [symbol] represents the covalent attachment to S*.

Provided herein are antibodies having biocompatible polymeric moieties (BPMs) covalently attached via cleavable moieties, providing adjustable magntitudes of Fc receptor interaction. The resulting antibodies initially exhibit decreased Fc receptor binding upon administration, but exhibit an increase in Fc receptor affinity over time.

Some embodiments provide a MEF antibody, wherein: the MEF antibody has 1, 2, 3, or 4 reduced interchain disulfide bonds and 2, 4, 6, or 8 biocompatible polymeric moieties (BPMs), respectively; wherein each BPM is covalently attached to each sulfur atom of the cysteine residues of each reduced interchain disulfide bond of the MEF antibody via a cleavable moiety; and wherein the MEF antibody exhibits time-dependent reduction in FcR binding, and thus a corresponding time-dependent reduction in an effector function, relative to that of an equivalent antibody.

Some embodiments provide a composition comprising a distribution of MEF antibodies, as described herein. In some embodiments, the MEF antibodies of the distribution differ primarily in the number of covalently attached BPMs.

Some embodiments provide a composition comprising a first population of an MEF antibody composition; a second population of an MEF antibody; and at least one pharmaceutically acceptable carrier; wherein the BPMs present in the first population of MEF antibodies are different than the BPMs present in the second population of MEF antibodies.

Some embodiments provide a composition comprising a first population of an MEF antibody composition; a second population of an MEF antibody composition; and at least one pharmaceutically acceptable carrier; wherein the cleavable moieties present in the first population of MEF antibodies are different than the cleavable moieties present in the second population of MEF antibodies.

Some embodiments provide a method of decreasing the severity of an infusion related reaction in a subject associated with an antibody, comprising intravenously administering to the subject a composition comprising an MEF antibody; wherein the severity of the infusion related reaction is decreased from 1 to 4 units relative to intravenous administration of an equimolar amount of the antibody; and wherein the antibody is equivalent to the MEF antibody.

Some embodiments provide a method of reducing the incidence of and/or risk of developing an infusion related reaction in a subject associated with an antibody, comprising intravenously administering to the subject a composition comprising an MEF antibody; wherein the antibody is equivalent to the MEF antibody; and wherein the incidence of and/or risk of developing the infusion related reaction is reduced relative to intravenous administration of an equimolar amount of the antibody.

Some embodiments provide a method of reducing one or more symptoms of an infusion related reaction in a subject associated with an antibody, comprising intravenously administering to the subject a composition comprising an MEF antibody; wherein the antibody is equivalent to the MEF antibody; and wherein the one or more symptoms of the infusion related reaction are reduced relative to intravenous administration of an equimolar amount of the antibody.

Some embodiments provide an MEF antibody having the structure:

Ab-(S*—X-BPM)ₚ

wherein: each S* is a sulfur atom from a cysteine residue of a reduced interchain disulfide bond of the MEF antibody; each X is a cleavable moiety; each BPM is a polyethylene glycol moiety, a polyketal moiety, a polyglycerol moiety, a polysaccharide moiety, a polysarcosine moiety, a polypeptide moiety, or a polyzwitterionic moiety; subscript p is 2, 4, 6, or 8; and Ab represents the remainder of the antibody.

Some embodiments provide a method of decreasing the Cₘₐₓ of an active antibody, comprising intravenously administering to a subject a composition comprising a MEF antibody;

wherein the active antibody is equivalent to the MEF antibody; wherein the Cₘₐₓ of the active antibody after intravenous administration of the MEF antibody composition is decreased relative to the Cₘₐₓ after intravenous administration of an equimolar amount of the active antibody.

Some embodiments provide a method of delaying maximal Fc gamma receptor IIIa binding of an antibody, comprising intravenously administering to a subject a composition comprising an MEF antibody to the subject in need thereof; wherein the antibody is equivalent to the MEF antibody; and wherein the MEF antibody delays binding to Fc gamma receptor IIIa relative to the antibody.

Some embodiments provide a method of selectively increasing binding of an antibody to Fc gamma receptor IIIa in a target cell in a subject, comprising intravenously administering a composition comprising an MEF antibody to the subject; wherein the antibody is equivalent to the MEF antibody; and wherein the ratio of the MEF antibody (i) bound to Fc gamma receptor Ma at the target cell and (ii) bound to Fc gamma receptor IIIa systemically is increased relative to the ratio of the antibody (i) bound to Fc gamma receptor IIIa at the target cell and (ii) bound to Fc gamma receptor IIIa systemically.

Some embodiments provide a method of reducing systemic Fc gamma receptor IIIa activation in a subject after administration of an antibody, comprising intravenously administering a composition comprising an MEF antibody to the subject, wherein the antibody is equivalent to the MEF antibody; and wherein the administration of the MEF antibody provides reduced systemic activation of Fc gamma receptor IIIa relative to intravenous administration of an equimolar amount of the antibody.

Some embodiments provide a method of decreasing systemic cytokine production in a subject after administration of an antibody, comprising intravenously administering a composition comprising an MEF antibody to the subject; wherein the antibody is equivalent to the MEF antibody; and wherein administration of the composition comprising the MEF antibody decreases systemic cytokine production relative to intravenous administration of an equimolar amount of the antibody.

Some embodiments provide a method of selectively activating an antibody, comprising intravenously administering a composition comprising a distribution of MEF antibodies; wherein at least about 10% of the BPMs are cleaved from the MEF antibody within about 12 hours and at least about 25% of the BPMs are cleaved from the MEF antibody within 48 hours.

Some embodiments provide a method of selectively activating an antibody, comprising intravenously administering a composition comprising a distribution of MEF antibodies; wherein at least about 25% of the BPMs are cleaved from the MEF antibody within about 12 hours and at least about 75% of the BPMs are cleaved from the MEF antibody within 24 hours.

Some embodiments provide a method of selectively activating an antibody, comprising intravenously administering a composition comprising a distribution of MEF antibodies; wherein about 25% to about 75% of the BPMs are cleaved from the MEF antibody within about 48 hours.

Some embodiments provide a method of selectively activating an antibody, comprising intravenously administering a composition comprising a distribution of MEF antibodies; wherein about 25% to about 75% of the BPMs are cleaved from the MEF antibody within about 72 hours.

### BRIEF DESCRIPTION OF THE FIGURES

FIGS. 1A-B illustrates the stability of modified antibodies Anti-CD40-AF-12 (FIG. 1A) and Anti-CD40-AF-1 (FIG. 1B) in rat plasma as assessed by the WES capillary gel electrophoresis assay.

FIG. 2 illustrates FcgRIIIa NFAT activity and EC₅₀ values of various modified antibodies (Anti-CD40-WT, Anti-CD40-AF, ANTI-CD40-AF-10, ANTI-CD40-AF-14, ANTI-CD40-AF-15, and ANTI-CD40-AF-17) versus control hIgG1k.

FIGS. 3A-C illustrates three time courses of FcgRIIIa NFAT activity of modified antibodies (Anti-CD40-WT, Anti-CD40-AF, Anti-CD40-AF-1, Anti-CD40-AF-12, Anti-BCMA-WT, Anti-BCMA-AF, and Anti-BCMA-AF-12) versus control hIgG1k in rat plasma (time in h).

FIG. 4 illustrates saturation binding of various modified antibodies (Anti-TIGIT-WT, Anti-TIGIT-AF, Anti-TIGIT-null Fc, Anti-TIGIT-AF-1 and Anti-TIGIT-AF-12) to CHO cells expressing human FcgRIIIa.

FIGS. 5A-D illustrates cytokine activity of human PBMCs dosed with various modified antibodies (Anti-CD40-WT, Anti-CD40-AF, Anti-CD40-AF-NEM, Anti-CD40-AF-12 and Anti-CD40-AF-19) versus control hIgG1k. The cytokine activity was measured for IP-10 (FIG. 5A), MIP-1b (FIG. 5B), TNFa (FIG. 5C), and MIP-1a (FIG. 5D).

FIGS. 6A-D illustrates cytokine production 2, 8, 24, 48, 72 hours post-dose in mice with transgenic human CD40 treated with various modified antibodies (Anti-CD40-WT, Anti-CD40-AF, Anti-CD40-AF-9, Anti-CD40-AF-10, and Anti-CD40-AF-12) versus untreated mouse. The cytokine production was measured for MCP-1 (FIG. 6A), KC (FIG. 6B), IP-10 (FIG. 6C), and MIP-1b (FIG. 6D).

FIGS. 7A-B illustrates cytokine production 2, 8, 24, 48, 72 hours post-dose in mice with transgenic human CD40 treated with various modified antibodies (Anti-CD40-WT, Anti-CD40-AF, Anti-CD40-AF-1, Anti-CD40-AF-2, and Anti-CD40-AF-9) versus untreated mouse. The cytokine production was measured for MCP-1 (FIG. 7A) and IP-10 (FIG. 7B).

FIGS. 8A-B illustrates FcgRIIIa NFAT activity of various modified antibodies (Anti-CD40-WT, Anti-CD40-AF, Anti-CD40-AF-NEM 4 load, Anti-CD40-AF-1 with 2, 4, and 6-load (FIG. 8A), and Anti-CD40-AF-12 with 2, 4, 5, 6, 7 and 7.5 load) (FIG. 8B) versus control hIgG1k.

FIGS. 9A-B illustrates FcgRIIIa NFAT activity of various modified antibodies: obinituzumab-WT, obinituzumab-AF, and obinituzumab-AF-12 in FIG. 9A; and rituximab, and rituximab-AF, and rituximab-AF-12 in FIG. 9B; versus control hIgG1k.

FIG. 10 illustrates in vivo mean tumor volume data for various modified antibodies (Anti-TIGIT-WT, Anti-TIGIT-AF, Anti-TIGIT-null Fc, Anti-TIGIT-AF-1, and Anti-TIGIT-AF-12) in a syngeneic mouse tumor model bearing subcutaneous CT26WT tumors.

FIG. 11 illustrates in vivo mean tumor volume data for various modified antibodies (Anti-CD40-WT, Anti-CD40-AF, Anti-CD40-AF-1, Anti-CD40-AF-9, and Anti-CD40-AF-12) in a mouse tumor model bearing subcutaneous A20 tumors.

FIG. 12 illustrates results from a FcγIIIa binding assay with antibodies containing PEG and oligopeptide-PEG functionalizations.

FIG. 13 summarizes changes in PEG oligomer to antibody ratios following administration to rats.

FIG. 14 overviews CD16a activity of PEG12 functionalized antibodies at multiple time-points following dosing in rats.

FIGS. 15A-D summarize results from CD16a binding assays for multiple concentrations of antibodies with various combinations of S239D, A330L, I332E, and PEG BPM modifications. FIGS. 15A-B provide results for antibodies with and without PEG12. FIG. 15C provides results for S239D I332E double mutants with and without PEG12 functionalizations. FIG. 15D provides results for S239D A330L I332E triple mutants with and without PEG12 functionalizations.

FIG. 16 summarizes MCP-1 levels in cynomolgus macaques prior to (x-axis ‘Pre’) and following non-PEGylated (Anti-CD40-SEA) and PEGylated (Anti-CD40-SEA-MC-PEG12) antibody administration.

FIG. 17 summarizes antibody levels in cynomolgus macaques following administration of non-PEGylated (Anti-CD40-SEA) and PEGylated (Anti-CD40-SEA-MC-PEG12) antibodies.

FIG. 18 summarizes B-cell levels in cynomolgus macaques following administration of non-PEGylated (Anti-CD40-SEA) and PEGylated (Anti-CD40-SEA-MC-PEG12) antibodies.

FIG. 19 summarizes MIP-1β levels in cynomolgus macaques prior to (x-axis ‘Pre’) and following non-PEGylated (Anti-CD40-SEA) and PEGylated (Anti-CD40-SEA-MC-PEG12) antibody administration.

FIG. 20 summarizes IL-1RA levels in cynomolgus macaques prior to (x-axis ‘Pre’) and following non-PEGylated (Anti-CD40-SEA) and PEGylated (Anti-CD40-SEA-MC-PEG12) antibody administration.

FIG. 21 outlines activity of an MEF antibody consistent with aspects of the present disclosure.

FIG. 22 depicts BPM coupling to interchain disulfide bond-derived thiols of an antibody consistent with the present disclosure.

### DETAILED DESCRIPTION

The in vivo toxicity of antibodies is often linked to their pharmacokinetics and affinity for their cognate Fc receptors. For many antibody-based treatments, Fc receptor-mediated effector functions simultaneously activate immune responses requisite for treatment efficacy and generate systemic toxicities which can limit dosing. As a means for controlling Fc receptor activation, the antibodies described herein can include cleavable biocompatible polymeric moieties (BPMs) which decrease Fc receptor binding in a time dependent manner. In many such cases, the resulting modulated antibodies initially exhibit decreased Fc receptor binding upon administration, but exhibit an increase in Fc receptor affinity over time.

After administration of such antibodies, the cleavable moieties, which covalently link the BPMs to the MEF antibody, are cleaved over time. Cleavage of the cleavable moieties releases the BPMs, a fragment of the BPMs, and/or an adduct formed from part of a cleavable moiety and a BPM. Each cleavage event thus removes an impediment to binding an Fc receptor, such that when all the BPMs have been released the antibodies can interact with an Fc receptor in substantially the same way as the equivalent antibody lacking the BPMs. The cleavable moieties can be selected to provide different time courses and conditions for cleavage. Thus, these antibodies can exhibit extended half-lives relative to traditional therapeutic antibodies or equivalent antibodies lacking the BPMs, without the need for extended infusion periods. This approach can enable tunable antibody activation, as well as tuning of an antibody's half-life, while maintaining activity and reducing systemic cytokine release and its concomitant adverse effects.

### Definitions

Unless otherwise defined, all technical and scientific terms used herein have the same meaning as commonly understood by one of ordinary skill in the art to which this disclosure belongs. Methods and materials are described herein for use in the present application; other, suitable methods and materials known in the art in some aspects of this disclosure are also used. The materials, methods, and examples are illustrative only and not intended to be limiting. All publications, patent applications, patents, sequences, database entries, and other references mentioned herein are incorporated by reference in their entireties. In case of conflict, the present specification, including definitions, will control. When trade names are used herein, the trade name includes the product formulation, the generic drug, and the active pharmaceutical ingredient(s) of the trade name product, unless otherwise indicated by context.

The term “biocompatible polymeric moiety” (BPM) as used herein, refers to a polyethylene glycol moiety, a polyketal moiety, a polyglycerol moiety, a polysaccharide moiety, a polysarcosine moiety, a polypeptide moiety, and/or a polyzwitterionic moiety, as described herein. In many cases, BPMs do not include polymeric groups that are linked to drug molecules. BPMs can be monodisperse, having a very similar degree of polymerization or relative molecular mass (typically purified from a heterogeneous mixture), or polydisperse, containing polymer chains of unequal length, and a distribution of molecular weights. As used herein, the term “polydispersity” can denote a ratio of weight average molecular weight to number average molecular weight for a collection of BPMs. Monodisperse BPMs can have a polydispersity index of about 1.0 (e.g., about 1.01, about 1.02, about 1.03, about 1.04, about 1.05, about 1.06, about 1.07, about 1.08, about 1.09, at most about 1.09, at most about 1.05, or at most about 1.03), while polydisperse BPMs can have a polydispersity index of at least 1.10 (e.g., at least about 1.10, at least about 1.11, at least about 1.12, at least about 1.13, at least about 1.14, at least about 1.15, at least about 1.16, at least about 1.17, at least about 1.18, at least about 1.19, at least about 1.20, at least about 1.3, at least about 1.4, at least about 1.5, at least about 2, at least about 2.5, or at least about 3).

The term “polyethylene glycol moiety” (PEG) as used herein, refers to a polymer of repeating ethylene glycol units that can be straight chain or branched. A branched PEG moiety can include a backbone, such as an alkyl chain. In many aspects disclosed herein, a PEG moiety has between 2 and 100 ethylene glycol monomers, denoted as PEG2-PEG100, for example, PEG2-PEG20, PEG4-PEG40, PEG8-PEG60, PEG10-PEG80, PEG12-PEG100, PEG2-PEG20, PEG2-PEG12, PEG4-PEG20, PEG4-PEG12, PEG8-PEG20, PEG8-PEG12, or PEG20-PEG76. The size of a PEG moiety can also be expressed by its average molecular weight, rather than a specific number of PEG units, for example, about 100 Da to about 5,000 Da. Straight chain PEG moieties can be represented by the structure

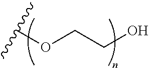

having “n” PEG units. Branched PEG can be represented by the following structures, where “n” represents the number of PEG units:

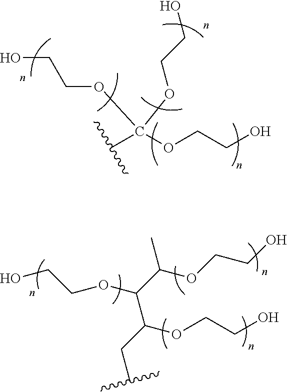

The term “polyketal moiety” as used herein, refers to a polymer of repeating ketal units. The size of a polyketal moiety can be expressed by the number of ketal units (e.g., 2-20), or can be expressed by its average molecular weight. Polyketals include, but are not limited to poly(dimethoxyacetone ketal) and “n” units of

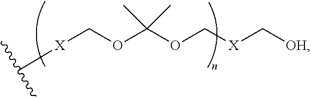

where X is phenylene or cyclohexylene, and “n” represents the number of ketal units.

The term “polyglycerol moiety” as used herein, refers to a polymer of repeating glycerol units. Polyglycerol moieties can be straight chain or branched, and can have 2-48 glycerol units. The size of a polyglycerol moiety can also be expressed by its average molecular weight, for example, about 160 Da to about 3,600 Da. Polyglycerol moieties can be α-functionalized, ω-functionalized, or backbone functionalized. An exemplary polyglycerol moiety is

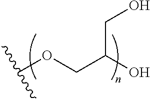

where “n” represents the number of glycerol units.

The term “polysaccharide moiety” as used herein, refers to a chain of independently selected saccharide units. A polysaccharide moiety can be straight chain or branched, and can include one or more α-1,4 glycosidic linkages, β-1,4 glycosidic linkages, α-1,6 glycosidic linkages, β-1,6 glycosidic linkages, and α-1, β-2 glycosidic linkages. Exemplary saccharide monomers include, but are not limited to glucose, fructose, galactose, arabinose, ribose, gulose, mannose, fucose, rhamnose, and combinations thereof. Polysaccharide moieties can include from 2-12 saccharide units, for example, 1, 2, 3, 4, 5, 6, 7, 8, 9, 10, 11, 12 saccharide units, and/or can be from about 350 Daltons to about 3,500 Daltons.

The term “polysarcosine moiety” as used herein, refers to a polymer comprising repeating sarcosine (N-methyglycine) units. In some cases, a polysarcosine moiety can have between 2 and 36 discrete sarcosine units, and/or be from about 250 Daltons to about 3,000 Daltons. In some cases, a polysarcosine moieties can be represented as

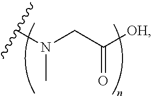

where “n” represents the number of sarcosine units.

The term “polypeptide moiety” as used herein refers to a branched or an unbranched chain of independently selected amino acids (including natural and non-natural amino acids). Exemplary amino acids include arginine, histidine, lysine, aspartic acid, glutamic acid, serine, threonine, asparagine, glutamine, cysteine, glycine, proline, alanine, valine, leucine, isoleucine, methionine, phenylalanine, tyrosine, tryptophan, ornithine, citrulline, and beta-alanine. Polypeptide moieties can have between 4 and 60, between 10 and 50, or between 10 and 30 discrete amino acids, and/or be from about 500 Daltons to about 7,000 Daltons. Polypeptide moieties can be represented as -(AA)ₙ- where “n” represents the number of amino acids.

The term “polyzwitterionic moiety” as used herein, refers to polymers that bear, within their constitutional repeat unit, the same number of anionic and cationic groups, such that each polyzwitterionic moiety has a net zero charge at physiological pH, for example, betaine or choline-based groups such as polycarboxybetaine and carboxybetaine acrylamide. See Laschewsky, Polymers 2014: 6; 1544-1601 and Zhang, et al., Proc. Nat. Acad. Sci., Vol. 112, No. 39, pp. 12046-12051 (2015), each of which are hereby incorporated by reference in their entireties. Polyzwitterionic moieties can have between 2-100 monomers and/or be between about 300 Daltons and about 5,000 Daltons.

“Physiological pH,” as used herein, can refer to a pH of about 7.3 to about 7.5.

The term “cleavable moiety” as used herein, refers to a chemical moiety that cleaves under a physiological condition. The cleavable moiety may cleave under multiple physiological conditions, for example in multiple locations or microenvironments within human body, or under a specific physiological condition, such as a tumor microenvironment. A cleavable moiety can connect an antibody and a BPM, such that when the cleavable moiety is cleaved, the BPM to which it is attached is released from the antibody. In many cases, cleavable moieties do not contain, nor are they attached to, drug molecules.

As used herein, the term “hydrolysable group” can refer to a moiety which undergoes spontaneous hydrolytic cleavage under a specific condition or range of conditions. For example, a hydrolysable group may be inert in neutral and basic solutions, but may undergo hydrolytic cleavage in days, hours, minutes, or seconds under acidic conditions. In some cases, a hydrolysable group is configured to undergo hydrolytic cleavage in a particular physiological environment, such as blood (e.g., peripheral blood) or oxidative (e.g., lysosomal) or reductive (e.g., cytoplasmic) intracellular compartments. In some cases, a hydrolysable group is configured for catalytic cleavage, for example by enzymes present in a specific organism (e.g., humans) or tissues (e.g., metabolically active tissues such as liver, kidney, or brain). A hydrolysable group can be configured for cleavage by a range of enzymes, or by a specific enzyme. For example, a hydrolysable group can comprise an oligopeptide of the sequence arginine-arginine-valine-arginine, for which human furin may have high cleavage activity. A hydrolysable group can be configured for cleavage within a particular environment, such as human cell endosomes or lysozomes. In such cases, the hydrolysable group may be stable outside of the environment in which it is configured for cleavage. For example, a hydrolysable group may be stable in circulation within peripheral blood, but hydrolytically cleave upon uptake into a cell. Examples of hydrolysable groups include disulfides, organophosphates such as phosphate esters, thiophosphates, and dithiophosphates, carbamates, carbonates, thioesters, quaternary amines, ureas, organosulfates, diorganosulfates, certain amides and esters, and peptides with protease cleavage sites.

The term “antibody” as used herein covers intact antibodies including monoclonal antibodies, polyclonal antibodies, monospecific antibodies, and multispecific antibodies (e.g., bispecific antibodies). The term “antibody” can also include portions of antibodies or non-naturally occurring constructs, including V_{H} domains, Fab domains, scFv constructs, diabodies, triabodies, tetrabodies, minibodies, nanobodies, and fusion and synthetic constructs thereof. The term “antibody” can include reduced forms of antibodies and antigen binding antibody fragments in which one or more of the interchain disulfide bonds are disrupted, that exhibit the desired biological activity and provided that the antigen binding antibody fragments have both a functional Fc receptor binding region, and the requisite number of attachment sites for the desired number of attached BPMs. The native form of an antibody is a tetramer and consists of two identical pairs of immunoglobulin chains, each pair having one light chain and one heavy chain. In each pair, the light and heavy chain variable domains (VL and VH) are together primarily responsible for binding to an antigen. The light chain and heavy chain variable domains consist of a framework region interrupted by three hypervariable regions, also called “complementarity determining regions” or “CDRs.” The constant regions may be recognized by and interact with the immune system. (see, e.g., Janeway et al., 2001, Immuno. Biology, 5th Ed., Garland Publishing, New York). An antibody includes any isotype (e.g., IgG, IgE, IgM, IgD, and IgA) or subclass (e.g., IgG1, IgG2, IgG3, IgG4, IgA1 and IgA2) thereof. The antibody is derivable from any suitable species. In some aspects, the antibody is of human or murine origin, and in some aspects the antibody is a human, humanized or chimeric antibody.

The term “therapeutic antibody” as used herein refers to an antibody, as described herein, that serves to deplete target cells to exert a therapeutic effect. For example, a therapeutic antibody can bind to an antigen present on a target cell, such as a tumor-specific antigen, ultimately resulting in the death of that cell.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. The modifier “monoclonal” indicates the character of the antibody as being obtained from a substantially homogeneous population of antibodies and is not to be construed as requiring production of the antibody by any particular method.

An “intact antibody” is one which comprises an antigen-binding variable region as well as a light chain constant domain (C_{L}) and heavy chain constant domains, C_{H}1, C_{H}2, C_{H}3 and C_{H}4, as appropriate for the antibody class. The constant domains are either native sequence constant domains (e.g., human native sequence constant domains) or amino acid sequence variant thereof.

An “antibody fragment” comprises a portion of an intact antibody that includes the antigen-binding or variable region thereof. Antibody fragments of the present disclosure include at least one cysteine residue that provides a site for attachment of a cleavable moiety and/or cleavable moiety-BPM construct. In some embodiments, an antibody fragment includes Fab, Fab′, F(ab′)₂.

An “antigen” is an entity to which an antibody specifically binds.

A “modulated effector function (MEF) antibody” refers to an antibody (as described herein) with one or more modifications which affects its effector function. For example, an MEF antibody as disclosed herein can comprise BPMs or fragments of one or more BPMs (e.g., the portion of a cleavable moiety that remains covalently attached to the antibody after cleavage) bound to the antibody at sulfur atom from a cysteine residue of a reduced interchain disulfide bond of the antibody.

An “equivalent antibody” refers to an antibody that is substantially identical to a corresponding MEF antibody, but lacks the reduced interchain disulfide bonds, cleavable moieties, and BPMs present in the MEF antibody.

The term “time-dependent reduction” as used herein refers to the reduction of a parameter, property, and/or biological process from an initial state, where the reduction is reversed over time such that the initial state is partially or completely restored. In the context of the present disclosure the degree in reduction of the binding affinity of the Fc region of a MEF antibody to the antibody's cognate FcR is dependent on the structure and number of BPMs that were covalently attached to the antibody. For a defined BPM structure, the initial decrease in FcR binding affinity, and thus the initial decrease in the effector function relative to the effector function provided by the equivalent antibody, becomes greater as the number of BPMs covalently attached to the antibody is increased. Loss of the BPMs of the MEF antibody over time, for example, upon exposure of the MEF antibody to a biological media is related to the kinetics at which the FcR binding affinity is partially or completely restored to that of the equivalent antibody. A parameter, property, and/or biological process related to the effector function that is also reduced from its initial state likewise experiences a time-dependent reduction, the kinetics of which are not necessarily in lockstep with that of the FcR binding affinity.

The terms “specific binding” and “specifically binds” mean that the antibody or antibody fragment thereof will bind, in a selective manner, with its corresponding target antigen and not with a multitude of other antigens. Typically, the antibody or antibody fragment binds with an affinity of at least about 1×10⁻⁷ M, for example, 10⁻⁸ M to 10⁻⁹ M, 10⁻¹⁰ M, 10⁻¹¹ M, or 10⁻¹² M and binds to the predetermined antigen with an affinity that is at least two-fold greater than its affinity for binding to a non-specific antigen (e.g., BSA, casein) other than the predetermined antigen or a closely-related antigen.

The term “maximal Fc gamma receptor binding” means the binding interaction of an antibody with an Fc gamma receptor necessary to elicit a full (e.g., 100%) or close to full response from the receptor. The binding interaction of an antibody with an Fc gamma receptor can be delayed, decreased, or otherwise modified by the addition of one or more BPMs as described herein.

The term “inhibit” or “inhibition of” means to reduce by a measurable amount, or to prevent entirely (e.g., 100% inhibition).

The term “therapeutically effective amount” refers to an amount of a MEF antibody described herein that is effective to treat a disease or disorder in a mammal. For example, in the case of cancer, the therapeutically effective amount of a MEF antibody provides one or more of the following biological effects: reduction of the number of cancer cells; reduction of tumor size; inhibition (i.e., slow to some extent and preferably stop) of cancer cell infiltration into peripheral organs; inhibition (i.e., slow to some extent and preferably stop) of tumor metastasis; inhibition, to some extent, of tumor growth; and/or relief to some extent one or more of the symptoms associated with the cancer. For cancer therapy, efficacy in some aspects is measured by assessing the time to disease progression (TTP) and/or determining the response rate (RR).

The terms “cancer” and “cancerous” refer to or describe the physiological condition or disorder in mammals that is typically characterized by unregulated cell growth. A “tumor” comprises multiple cancerous cells.

An “autoimmune disorder” herein is a disease or disorder arising from and directed against an individual's own tissues or proteins.

“Subject” as used herein refers to an individual to which a MEF antibody is administered. Examples of a “subject” include, but are not limited to, a mammal such as a human, rat, mouse, guinea pig, non-human primate, pig, goat, cow, horse, dog, cat, bird, and fowl. Typically, a subject is a rat, mouse, dog, non-human primate, or human. In some aspects, the subject is a human in need of a therapeutically effective amount of a MEF antibody.

The terms “treat” or “treatment,” unless otherwise indicated or implied by context, refer to therapeutic treatment and prophylactic measures to prevent relapse, wherein the object is to inhibit or slow down (lessen) an undesired physiological change or disorder, such as, for example, the development or spread of cancer. For purposes of the present disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” in some aspects also means prolonging survival as compared to expected survival if not receiving treatment. Those in need of treatment include those already with the condition or disorder and in some aspects further include those prone to have the condition or disorder.

In the context of cancer, the term “treating” includes any or all of: inhibiting growth of tumor cells, cancer cells, or of a tumor; inhibiting replication of tumor cells or cancer cells, lessening of overall tumor burden or decreasing the number of cancerous cells, and ameliorating one or more symptoms associated with the disease.

In the context of an autoimmune disorder, the term “treating” includes any or all of: inhibiting replication of cells associated with an autoimmune disorder state including, but not limited to, cells that produce an autoimmune antibody, lessening the autoimmune-antibody burden and ameliorating one or more symptoms of an autoimmune disorder.

The term “salt,” as used herein, refers to organic or inorganic salts of a compound, such as a BPM, such as those described herein, or a MEF antibody, as described herein. In some aspects, the compound contains at least one amino group, and accordingly acid addition salts can be formed with the amino group. Exemplary salts include, but are not limited to, sulfate, trifluoroacetate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A salt may involve the inclusion of another molecule such as an acetate ion, a succinate ion or other counterion. The counterion may be any organic or inorganic moiety that stabilizes the charge on the parent compound. Furthermore, a salt has one or more than one charged atom in its structure. In instances where there are multiple charged atoms as part of the salt multiple, counter ions are sometimes present. Hence, a salt can have one or more charged atoms and/or one or more counterions. A “pharmaceutically acceptable salt” is one that is suitable for administration to a subject as described herein and in some aspects includes salts as described by P. H. Stahl and C. G. Wermuth, editors, Handbook of Pharmaceutical Salts: Properties, Selection and Use, Weinheim/Zürich:Wiley-VCH/VHCA, 2002, the list for which is specifically incorporated by reference herein.

The term “alkyl” refers to a straight chain or branched, saturated hydrocarbon having the indicated number of carbon atoms (e.g., “C₁-C₈ alkyl” or “C₁-C₁₀” alkyl have from 1 to 8 or 1 to 10 carbon atoms, respectively) that is unsubstituted unless indicated otherwise explicitly or by context. When the number of carbon atoms is not indicated, the alkyl group has from 1 to 6 carbon atoms. Representative straight chain “C₁-C₈ alkyl” groups include, but are not limited to, methyl, ethyl, n-propyl, n-butyl, n-pentyl, n-hexyl, n-heptyl and n-octyl; while branched C₁-C₈ alkyls include, but are not limited to, isopropyl, sec-butyl, isobutyl, tert-butyl, isopentyl, and 2-methylbutyl.

The term “alkylene” refers to a bivalent saturated branched or straight chain hydrocarbon of the stated number of carbon atoms (e.g., a C₁-C₆ alkylene has from 1 to 6 carbon atoms) that is unsubstituted unless indicated otherwise explicitly or by context, and having two monovalent radical centers derived by the removal of two hydrogen atoms from the same or two different carbon atoms of the parent alkane. Typical alkylene radicals include but are not limited to: methylene (—CH₂—), 1,2-ethylene (—CH₂CH₂—), 1,3-propylene (—CH₂CH₂CH₂—), 1,4-butylene (—CH₂CH₂CH₂CH₂—), and the like.

The term “alkoxy” refers to an alkyl group, as defined herein, which is attached to a molecule via oxygen atom. For example, alkoxy groups include, but are not limited to methoxy, ethoxy, n-propoxy, iso-propoxy, n-butoxy, sec-butoxy, tert-butoxy, n-pentoxy and n-hexoxy.

The term “cycloalkylene” refers to a bivalent saturated cyclic hydrocarbon of the stated number of carbon atoms (e.g., a C₃-C₆ cycloalkylene has from 3 to 6 carbon atoms) that is unsubstituted unless indicated otherwise explicitly or by context, and having two monovalent radical centers derived by the removal of two hydrogen atoms from the same or two different carbon atoms of the parent cycloalkane. Typical cycloalkylene radicals include but are not limited to: 1,2-cyclopropylene, 1,3-cyclobutylene, 1,3-cyclopentlyene, 1,4-cyclohexylene, and the like.

The term “interchain disulfide bond,” in the context of an antibody or MEF antibody, as described herein, refers to a disulfide bond between two heavy chains, or a heavy and a light chain.

The term “about” when referring to a number or a numerical range means that the number or numerical range referred to is an approximation, for example, within experimental variability and/or statistical experimental error, and thus the number or numerical range may vary ±10% of the stated number or numerical range.

The term “interrupted” when referring to a particular functional group being inserted into an alkylene group, includes both interruption within the carbon chain of a straight chain or branched alkyl group, as well as at the terminus of the alkyl group. For example, a hexylene group interrupted with —NHC(═O)—, includes, but is not limited to —CH₂CH₂—NHC(═O)—CH₂CH₂CH₂CH₂— and —CH₂CH₂CH₂CH₂CH₂CH₂—NHC(═O)—.

The term “substantial” or “substantially” refers to a majority, i.e. >50% of a population, of a mixture or a sample, typically more than 50%, 55%, 60%, 65%, 70%, 75%, 80%, 85%, 90%, 91%, 92%, 93%, 94%, 95%, 96%, 97%, 98%, or 99% of a population. The term “substantially the same” or “substantially identical” when referring to a number or a numerical range, or to the sequence of an antibody, means that the number or numerical range referred to is an approximation, for example, within experimental variability and/or statistical experimental error, and thus the number or numerical range may vary ±5% of the stated number or numerical range.

### Antibodies

Aspects of the present disclosure provide a modulated effector function (MEF) antibody with an effector function diminishing modification. The effector function diminishing modification can be a biocompatible polymeric moiety (BPM). The BPM can affect a binding affinity (e.g., Fc receptor and complement binding affinities), pharmacokinetic properties (e.g., clearance rate), localization behavior, and cellular uptake of the antibody. As the properties imparted by the BPM can depend on its size, structure (e.g., branched versus linear) location, and number (e.g., 1 vs 8 BPMs on an antibody), BPM modifications can tune antibodies for broad ranges of applications. In many cases, the BPM does not affect or minimally affects antigen binding (e.g., does not block or minimally blocks antibody paratopes), but does diminish Fc binding activity (e.g., antibody binding affinity for FcγRI, FcγRII, FcγRIII, FcRn, and/or complement proteins).

In many cases, a BPM of the present disclosure is cleavable. Depending on BPM cleavage location and chemistry (for example, whether the cleavage leaves a scar on the antibody), cleavage of the BPM can partially or fully reverse its effects on antibody localization and activity (e.g., effector function activity). As a non-limiting example, an increased K_{D} for FcγRIII can be restored upon BPM cleavage from an antibody.

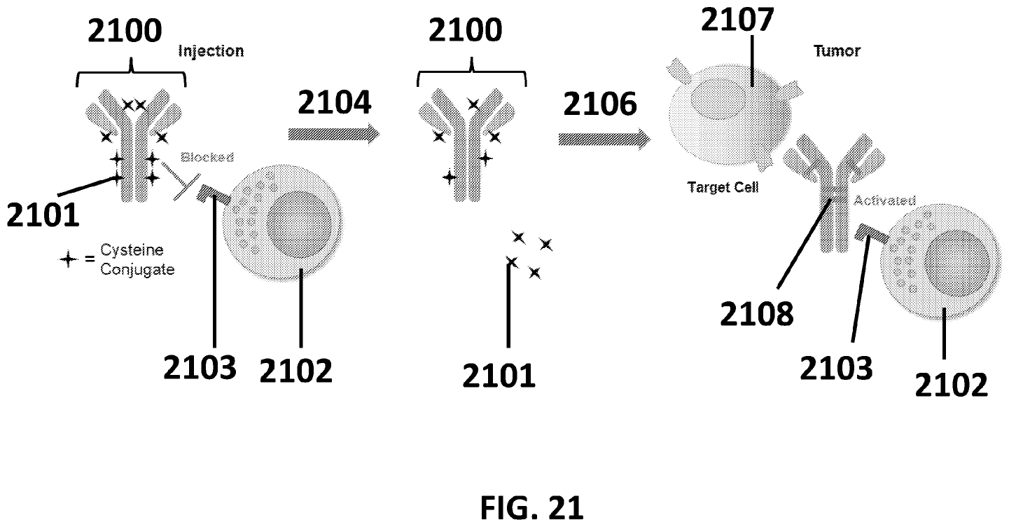

An illustrative example of in vivo activity of such a BPM-containing antibody is depicted in FIG. 21, in which, upon injection, the antibody 2100 contains BPMs 2101 which can diminish (e.g., block) interactions with immune cell 2102 Fc receptors 2103 (e.g., an FcγRIII receptor on a mast cell). During circulation 2104, the antibody can decouple from all or a portion of its BPMs, for example through hydrolytic cleavage. Following BPM loss 2106, the antibody 2100 can regain Fc receptor binding affinity, restoring or partially restoring its effector function. The antibody can bind to a target cell 2107, such as a tumor cell, such that its effector function is at least partially localized to a site of the target cell. In some cases, (e.g., when a BPM is coupled to a cysteine thiol), a disulfide bond can form following BPM loss.

Accordingly, cleavable BPMs can affect antibody activity (e.g., diminish effector function) in a time dependent manner. Without being bound by theory, a BPM can diminish effector function (e.g., Fc-FcgR binding) through multiple mechanisms (or combinations of mechanisms). In many cases, a BPM at least partially blocks an antibody Fc (e.g., through steric bulk), thereby preventing association with Fc receptors. Furthermore, a BPM can alter protein dynamics (e.g., solubility or physiological localization), thereby modifying the strength or prevalence of Fc receptor interactions. In some cases, BPM functionalizations (and in some cases accompanying disulfide bond reductions) can destabilize an antibody, thereby reducing the inherent binding affinity of its Fc for receptors.

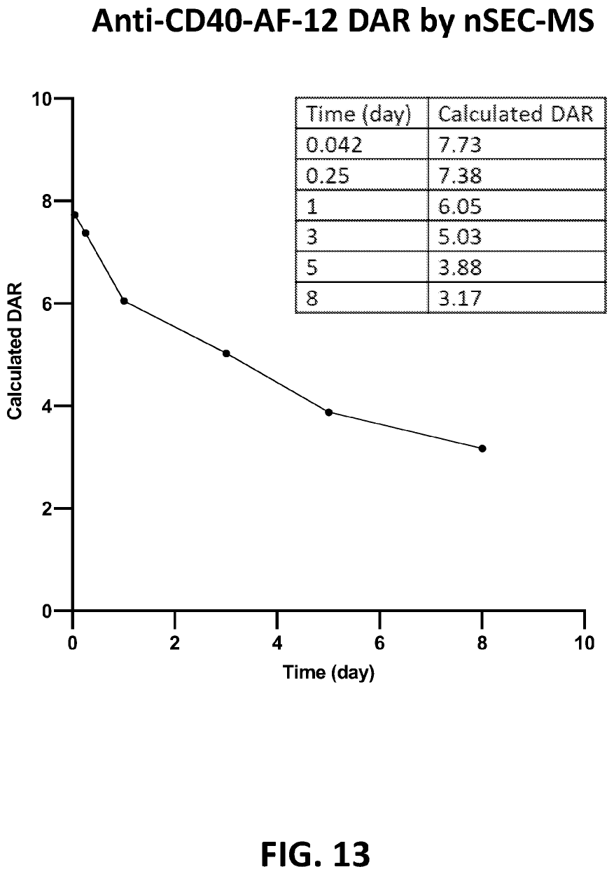

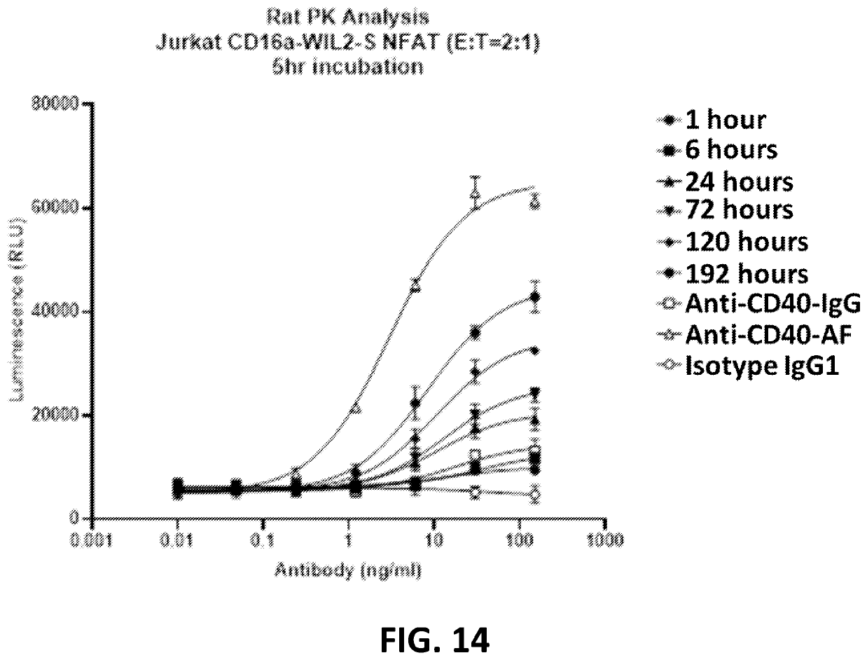

In some cases, a single BPM cleavage restores an activity (e.g., effector function), such that the BPM effectively functions as an “on-off” switch for that activity. In other cases, BPM cleavage restores only a portion of antibody activity. In some cases, such as those outlined in FIGS. 13 and 14, increased effector function may follow a relatively linear trend with respect to BPM cleavages. In other cases, successive BPM cleavages from antibodies with multiple BPMs can restore different degrees of activity. For example, an antibody with 8 BPMs may regain only 15% of its maximum effector function activity and greater than 99% of its antigen binding affinity after two BPM cleavages, but regain greater than 50% of its effector function activity following 4 BPM cleavages.

As disclosed herein, BPM cleavability can be exploited to impart time-dependence upon antibody activity (e.g., effector function activity), and to tune antibody activity to avoid toxic and off-target effects. Maintaining effector function and antigen binding activity through BPM modifications can require selection of BPM densities, sizes, structures, and cleavage rates, as well as antibody targets and structure. Tuning an MEF antibody to sequentially regain effector function in physiological conditions (e.g., as opposed to permanently losing effector function upon BPM modification) can require multiple BPMs which contribute to partial, but not complete, loss of effector function, as well as cleavage rates at least partially commensurate with or faster than clearance.

A surprising discovery disclosed herein is that, for many treatments, partially diminished effector function is optimal for eliciting localized (e.g., tumor-site) immune activation and avoiding antibody-induced systemic toxicities. Following from this observation, many antibodies of the present disclosure are configured to exhibit low or negligible effector function prior to BPM cleavage, and partial effector function following partial BPM cleavage (e.g., cleavage of a subset of BPMs coupled the antibody). Prior to BPM cleavage, an MEF antibody may have a binding affinity for an Fc receptor (e.g., FcγRI, FcγRIIa, FcγRIIb, FcγRIIIa, FcγRIIIb, or a receptor comprising at least 98% sequence identity to a receptor thereof) of at most 1%, at most 2%, most 5%, at most 10%, at most 15%, at most 20%, at most 25%, at most 30%, at most 40%, or at most 50% of that of an equivalent antibody lacking the BPM (e.g., a single BPM or a plurality of BPMs). In some cases, prior to BPM cleavage, the MEF antibody has between 1% and 30%, between 1% and 10%, between 2% and 20%, between 2% and 12%, between 5% and 25%, or between 10% and 30% of an effector function activity of an equivalent antibody lacking the BPM. In some cases, the MEF antibody has between 2% and 20% of an effector function activity of an equivalent antibody lacking the BPM.

In some cases, the MEF antibody has between 10% and 80%, between 10% and 30%, between 20% and 40%, between 20% and 50%, between 30% and 60%, or between 30% and 70% of the effector function activity of an equivalent antibody lacking the BPM following 192 hours incubation in 37° C. human plasma. In some cases, the MEF antibody has between 30% and 70% of the effector function activity of an equivalent antibody lacking the BPM following 192 hours incubation in 37° C. human plasma.

In some cases, prior to BPM cleavage, the MEF antibody has an FcγRIIIa binding affinity of at most 1%, at most 2%, most 5%, at most 10%, at most 15%, at most 20%, at most 25%, at most 30%, at most 40%, or at most 50% of that of an equivalent antibody lacking the BPM. In some cases, prior to BPM cleavage, the MEF antibody has between 1% and 30%, between 1% and 10%, between 2% and 20%, between 2% and 12%, between 5% and 25%, or between 10% and 30% of an FcγRIIIa binding affinity of an equivalent antibody lacking the BPM. In some cases, the MEF antibody has between 2% and 20% of an FcγRIIIa binding affinity of an equivalent antibody lacking the BPM. In some cases, the MEF antibody has between 10% and 80%, between 10% and 30%, between 20% and 40%, between 20% and 50%, between 30% and 60%, or between 30% and 70% of the FcγRIIIa binding affinity of an equivalent antibody lacking the BPM following 192 hours incubation in 37° C. human plasma. In some cases, the MEF antibody has between 30% and 70% of the FcγRIIIa binding affinity of an equivalent antibody lacking the BPM following 192 hours incubation in 37° C. human plasma.

In some cases, the MEF antibody is configured to regain between 10% and 50%, between 10% and 30%, between 25% and 40%, or between 30% and 50% of its Fc receptor binding affinity following cleavage of half (rounded up) of its BPMs (as a function of binding affinity of an equivalent antibody lacking the BPMs). In some cases, the MEF antibody is configured to undergo BPM cleavage at a rate of between 0.04 and 0.3 day⁻¹, between 0.075 and 0.2 day⁻¹, between 0.1 and 0.25 day⁻¹, between 0.1 and 0.5 day⁻¹, between 0.15 and 0.5 day⁻¹, or between 0.3 and 0.75 day⁻¹ during incubation in 37° C. human plasma. In some cases, the MEF antibody comprises a BPM cleavage rate of between 0.075 and 0.2 day⁻¹ (corresponding to BPM cleavage half-lives of between about 3.5 and about 9.25 days) during incubation in 37° C. plasma.

In some cases, the MEF antibody has a BPM cleavage rate which is at least 25%, at least 50%, at least 100%, at least 150%, at least 200%, at least 250%, at least 300%, at least 400%, or at least 500% of its physiological clearance rate during in vivo circulation in an adult human male. In specific cases, the MEF antibody comprises a cleavage rate of between about 50% and about 300% of its physiological clearance rate during in vivo circulation in an adult human male. In specific cases, the MEF antibody comprises a cleavage rate of between about 25% and about 200% of its physiological clearance rate during in vivo circulation in an adult human male.

The antibody can further comprise a modification in addition to the BPM, such as a mutation, tag, or post-translational modification. The modification can alter an antigen binding affinity, an effector function, a pharmacokinetic property of the antibody, or a combination thereof. In many cases, the modification increases MEF antibody effector function. When combined with an effector function-diminishing BPM, such as Fc-region PEGylation, the resultant antibody can exhibit enhanced activity localization and diminished systemic and off-target responses (e.g., increased blood cytokine levels). For example, a consortia of BPM-modified high effector function antibodies may localize to sites with high antigen concentrations (e.g., at sites with HER2+ metatstatic cancer cells), such that BPM cleavage, and concomitant effector function enhancement or restoration, disproportionately occur at target sites. Furthermore, for antibodies with multiple BPMs, an effector function enhancing modification may enable restoration of cytotoxic or phagocytic eliciting behavior with fewer BPM cleavages (e.g., only 1 of 8 BPMs may need to be cleaved to restore an effector function equivalent to that of an antibody analogue lacking the effector function enhancing modification).

Following from these observations, an MEF antibody of the present disclosure can comprise an effector function enhancing modification and an effector function diminishing modification, wherein the effector function diminishing modification comprises a biocompatible polymeric moiety (BPM) covalently attached to an amino acid or post-translational modification of the MEF antibody. In some cases, the effector function enhancing modification increases binding affinity for FcγRI, FcγRIIa, FcγRIIb, FcγRIIIa, FcγRIIIb, or a combination thereof. In some cases, the effector function enhancing modification increases binding affinity for FcγRIIIa. In some cases, the effector function enhancing modification comprises afucosylation, a bisecting N-acetyl glucosamine, an S298A Fc region mutation, an E333A Fc region mutation, a K334A Fc region mutation, an S239D Fc region mutation, an I332E Fc region mutation, a G236A Fc region mutation, an S239E Fc region mutation, an A330L Fc region mutation, a G236A Fc region mutation, a L234Y Fc region mutation, a G236W Fc region mutation, an S296A Fc region mutation, an F243 Fc region mutation, an R292P Fc region mutation, a Y300L Fc region mutation, a V305L Fc region mutation, a P396L Fc region mutation, or a combination thereof. In some cases, the effector function enhancing modification comprises afucosylation.

As used herein, the term ‘afucosylation’ can denote an absence of fucose on an antibody, can denote an absence of fucose on a plurality of antibodies (e.g., a unit dose of an antibody composition), or that a minor amount of fucose is incorporated into the complex N-glycoside-linked sugar chain(s) of a plurality of antibodies. Whereas in serum about 85% of IgG antibodies comprise fucose incorporated into N-glycoside-linked sugar chain(s), in various embodiments disclosed herein, less than about 30%, less than about 20%, less than about 15%, less than about 10%, less than about 5%, less than about 3% of the antibodies, less than about 2%, less than about 1%, or less than about 0.5% of antibodies of a plurality of antibodies have one or more fucose groups coupled thereto. In some embodiments, about 2% of the antibodies of the plurality have one or more fucose groups. In various embodiments, when less than 30% of the antibodies of a plurality of antibodies have fucose groups, the plurality of antibodies may be referred to as “nonfucosylated” or “afucosylated.” In some embodiments, at least 90%, at least 91%, at least 92%, at least 93%, at least 94%, at least 95%, at least 96%, at least 97%, at least 98%, or at least 99% of the antibodies of a plurality are afucosylated.

In certain embodiments, only a minor amount of a fucose analog (or a metabolite or product of the fucose analog) is incorporated into the complex N-glycoside-linked sugar chain(s) of the antibody. For example, in various embodiments less than about 30%, less than about 20%, less than about 15%, less than about 10%, less than about 5%, less than about 3%, less than about 2%, less than about 1%, or less than about 0.5% of antibodies of a plurality of antibodies have core fucosylation by a fucose analog or a metabolite or product of the fucose analog. In some embodiments, about 2% of antibodies of the plurality of antibodies have core fucosylation by a fucose analog or a metabolite or product of the fucose analog.

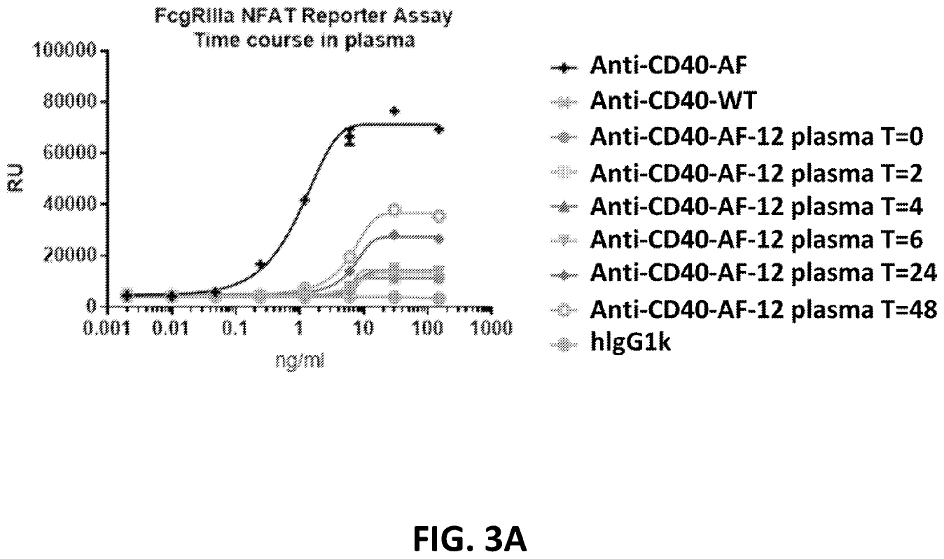

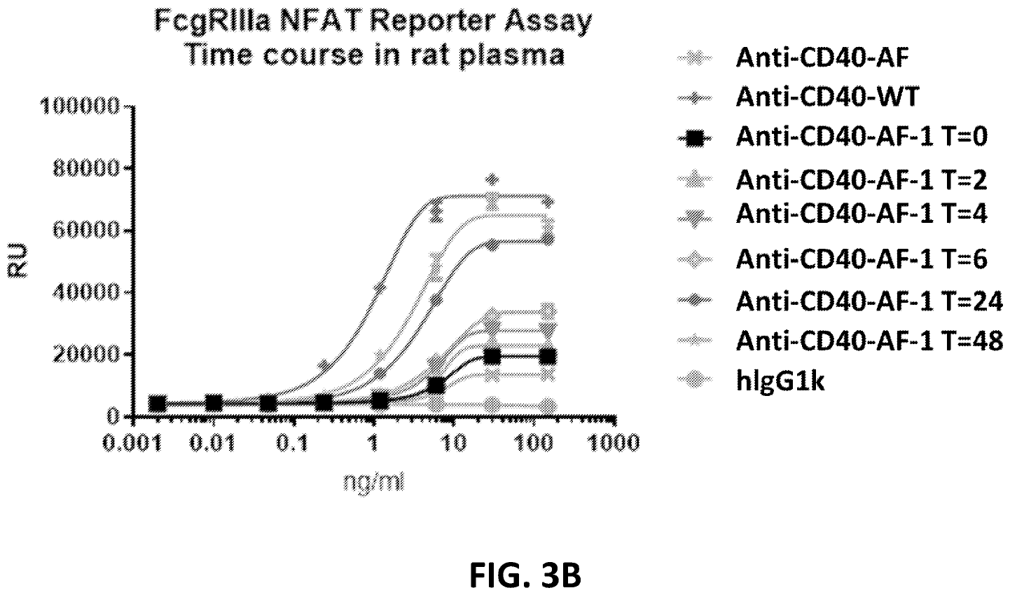

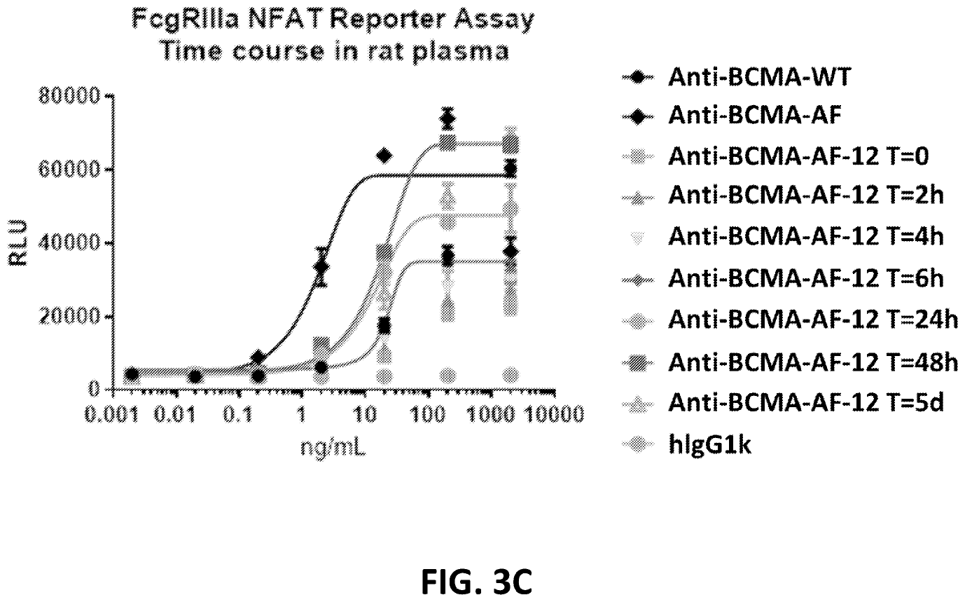

In some embodiments, less than about 30%, less than about 20%, less than about 15%, less than about 10%, less than about 5%, less than about 3%, less than about 2%, less than about 1%, or less than about 0.5% of antibodies of a plurality of antibodies have a fucose residue on a G0, G1, or G2 glycan structure. (See, e.g., Raju et al., 2012, MAbs 4: 385-391, FIG. 3.) In some embodiments, about 2% of the antibodies of the plurality of antibodies have a fucose residue on a G0, G1, or G2 glycan structure. In various embodiments, when less than 30% of the antibodies of a plurality of antibodies have a fucose residue on a G0, G1, or G2 glycan structure, the antibodies of the composition may be referred to as “afucosylated.” In some embodiments, at least 90%, at least 91%, at least 92%, at least 93%, at least 94%, at least 95%, at least 96%, at least 97%, at least 98%, or at least 99% of the antibodies of the plurality of antibodies lack fucose on a G0, G1, or G2 glycan structure. It should be noted that G0 glycans include G0-GN glycans, which can be monoantenary glycans with one terminal GlcNAc residue. G1 glycans include G1-GN glycans, which can be monoantenary glycans with one terminal galactose residue. G0-GN and G1-GN glycans can be fucosylated or non-fucosylated.

In some cases, the effector function diminishing modification is at least partially reversible. For example, in some cases, the effector function diminishing modification comprises a photoswitchable or chemically-switchable domain configured to interconvert the BPM between states which differentially alter effector function. In many cases, the BPM is configured for cleavage, which cleavage increases the effector function of the antibody. In some cases, the amino acid residue comprises a cysteine residue or a methionine residue. In some cases, the cysteine residue couples to the BPM to form a disulfide, a thioether, a thioallyl, a vinyl thiol, or a combination thereof. In some cases, the disulfide bond, the thioallyl bond, or the combination thereof is cleavable. In some cases, the methionine residue couples to the BPM through an S═N bond (e.g., as a sulfanimine). For example, the BPM can comprise an oxaziridine carboxamide, an oxaziridine ketone, or an oxaziridine carboxylate configured to couple to the methionine thioether.

In some cases, the BPM comprises an enzymatically cleavable group. In some cases, the enzymatically cleavable group is a protease cleavage sequence, a glycosidic group, a carbamate, a urea, a quaternary ammonium, or a combination thereof. In some cases, the enzymatically cleavable moiety is a protease cleavage sequence. In some cases, the protease cleavage sequence is a tumor-associated protease cleavage sequence. In some cases, the BPM comprises a moiety which cleaves under physiological conditions, such as a quaternary ammonia or a carbamate.

A BPM can be configured for cleavage at a site of attachment to an antibody. For example, a BPM can be coupled to an antibody-derived cysteine by a cleavable thioether (e.g., a cysteine-maleimide adduct), vinyl ether, or disulfide bond, such that cleavage completely removes the BPM from the antibody. Alternatively or in addition thereto, a cleavable group can be disposed within the BPM, such that a portion of the BPM remains attached to the antibody following its cleavage. In some cases, the BPM is configured for hydrolytic cleavage. In some such cases, BPM cleavage exhibits a first order rate dependence in plasma, cerebrospinal fluid, lymph, or another bodily fluid. In some cases, BPM cleavage is condition dependent. For example, a BPM may cleave slowly in plasma, but quickly within a low pH tumor microenvironment.

In some cases, the BPM is configured for enzymatic cleavage. When the enzymes for such cleavage are localized within specific tissues, cells, or sub-cellular compartments, the cleavable group can exhibit location specific or location enhanced cleavage, thereby primarily activating within target sites. Examples of BPM cleavable groups include protease and hydrolase cleavage sites. In some cases, the cleavable group includes a protease-cleavable peptide sequence. As non-limiting examples, the protease cleavage sequence can be a thrombin cleavage sequence, cathepsin cleavage sequence, a matrix metalloproteinase cleavage sequence, a PAR-1 activating peptide cleavage sequence, a kallikrein cleavage sequence, a granzyme cleavage sequence, a caspase cleavage sequence, an ADAM cleavage sequence, a calpain cleavage sequence, a prostate-specific antigen cleavage sequence, a fibroblast activation protein cleavage sequence, a dipeptidyl peptidase IV cleavage sequence, or a combination thereof. In some cases, the BPM cleavable group includes a cleavable glycosidic group. As non-limiting examples, the cleavable glycosidic group can comprise β-D-glucuronide, β-D-galactose, β-D-glucose, β-D-xylose, hexamaltose, β-L-gulose, β-L-allose, β-L-glucose, β-L-galactose, β-mannose-6-phosphate, β-L-fucose, α-E-mannose, β-D-fucose, 6-deoxy-β-D-glucose, β-mannose-6-phosphate, lactose, maltose, cellobiose, gentiobiose, maltotriose, β-D-GlcNAc, β-D-GalNAc, or a combination thereof. For example, the cleavable group can comprise β-glucuronidase or α-mannosidase-cleavage sites cleavable by lysosomal β-glucuronidases or α-mannosidases, thereby rendering the linker (L) inert prior to lysosomal uptake and cleavable subsequent to lysosomal uptake. In some cases, the BPM cleavable group comprises an enzymatically cleavable glycosidic bond, peptide bond, carbamate, or quaternary amine. In some cases, the enzyme for such cleavage is associated with a cancer cell, such as extracellular cathepsin.

In some embodiments, a cleavable moiety of a BPM is configured to undergo a secondary reaction which diminishes the cleavage rate of the BPM. For example, when the BPM comprises a succinimide (e.g., coupled to the antibody through a thioether bond), the succinimide can undergo a hydrolysis reaction to form a carboxylate and amide, which can slow the rate of cleavage (e.g., from an antibody-derived cysteine). In some cases, the cleavable moiety is configured to undergo the BPM cleavage at least at 1.5-times the rate of the secondary reaction during in vivo circulation in an adult human male. In some cases, the cleavable moiety is configured to undergo the BPM cleavage at least at 2-times the rate of the secondary reaction during in vivo circulation in an adult human male. In some cases, the cleavable moiety is configured to undergo the BPM cleavage at least at 2.5-times the rate of the secondary reaction during in vivo circulation in an adult human male. In some cases, the cleavable moiety is configured to undergo the BPM cleavage at least at 3-times the rate of the secondary reaction during in vivo circulation in an adult human male.

In some cases, the secondary reaction can inhibit or prevent full BPM cleavage (e.g., in 37° C. plasma or during in vivo circulation in an adult male human). In some cases, at least 10% of BPMs remain attached to the MEF antibody following one month of 37° C. plasma incubation. In some cases, at least 15% of BPMs remain attached to the MEF antibody following one month of 37° C. plasma incubation. In some cases, at least 20% of BPMs remain attached to the MEF antibody following one month of 37° C. plasma incubation. In some cases, at least 25% of BPMs remain attached to the MEF antibody following one month of 37° C. plasma incubation. In some cases, at least 30% of BPMs remain attached to the MEF antibody following one month of 37° C. plasma incubation. In some cases, at least 35% of BPMs remain attached to the MEF antibody following one month of 37° C. plasma incubation. In some cases, at least 60% of cleavable groups of BPMs which have remained attached to the MEF antibody following one month of 37° C. plasma incubation have undergone the secondary reaction. In some cases, at least 80% of cleavable groups of BPMs which have remained attached to the MEF antibody following one month of 37° C. plasma incubation have undergone the secondary reaction.

In some cases, at least 10% of BPMs remain attached to the MEF antibody following one month of in vivo circulation in an adult male human. In some cases, at least 15% of BPMs remain attached to the MEF antibody following one month of in vivo circulation in an adult male human. In some cases, at least 20% of BPMs remain attached to the MEF antibody following one month of in vivo circulation in an adult male human. In some cases, at least 25% of BPMs remain attached to the MEF antibody following one month of in vivo circulation in an adult male human. In some cases, at least 30% of BPMs remain attached to the MEF antibody following one month of in vivo circulation in an adult male human. In some cases, at least 35% of BPMs remain attached to the MEF antibody following one month of in vivo circulation in an adult male human. In some cases, at least 60% of cleavable groups of BPMs which have remained attached to the MEF antibody following one month of in vivo circulation in an adult male human have undergone the secondary reaction. In some cases, at least 80% of cleavable groups of BPMs which have remained attached to the MEF antibody following one month of in vivo circulation in an adult male human have undergone the secondary reaction.

Certain embodiments of the present disclosure provide a modulated effector function (MEF) antibody coupled to a plurality of biocompatible polymeric moieties (BPM) and an Fc which is at least partially blocked by the BPM; wherein a BPM of the plurality of BPMs is attached to a sulfur atom of a cysteine residue by a cleavable disulfide bond. Alternatively or in addition thereto, aspects of the present disclosure provide a modulated effector function (MEF) antibody coupled to a plurality of biocompatible polymeric moieties (BPM) which at least partially diminish an effector function of the MEF antibody; wherein a BPM of the plurality of BPMs is attached to a sulfur atom of a cysteine residue by a cleavable disulfide bond.

Certain embodiments of the present disclosure provide a modulated effector function (MEF) antibody coupled to a plurality of biocompatible polymeric moieties (BPM) and an Fc which is at least partially blocked by the BPM; wherein a BPM of the plurality of BPMs is attached to a methionine residue by a cleavable moiety. Alternatively or in addition thereto, certain embodiments of the present disclosure provide a modulated effector function (MEF) antibody coupled to a plurality of biocompatible polymeric moieties (BPM) which at least partially diminish an effector function of the MEF antibody; wherein a BPM of the plurality of BPMs is attached to a methionine residue by a cleavable moiety.

In some cases, a BPM at least partially diminishes an effector function of an antibody by at least partially blocking an Fc region of the antibody. In some cases, a BPM at least partially diminishes an effector function of an antibody by diminishing the antibody stability. For example, a BPM-functionalized antibody can denature a 5%, a 10%, a 15%, a 20%, or a 25% lower guanidinium concentration than an equivalent antibody lacking BPM functionalizations.

Certain embodiments of the present disclosure provide a modulated effector function (MEF) antibody comprising at least one Fc region and coupled to a plurality of biocompatible polymeric moieties (BPM) in a ratio to Fc regions of the at least one Fc region of between 6 and 10; wherein the plurality of biocompatible polymeric moieties comprise molecular weights of between 500 and 2500 Daltons (Da) and have cleavage rates of between 0.1 and 0.5 day′ in 37° C. human plasma. Certain embodiments of the present disclosure provide a modulated effector function (MEF) antibody, wherein the MEF antibody has a plurality of biocompatible polymeric moieties (BPMs), wherein each BPM is covalently attached to amino acid residues of the MEF antibody via cleavable moieties; and wherein the MEF antibody exhibits time-dependent reduction in FcR binding, and thus a corresponding time-dependent reduction in an effector function, relative to that of an equivalent antibody. In some cases, each BPM is covalently attached to sulfur-containing amino acid residues of the MEF antibody. In some cases, each BPM is covalently attached to cysteine residues of the MEF antibody. In some cases, at least a subset of the cysteine residues is derived from disulfide bonds in the MEF antibody prior to reduction and BPM coupling.

In particular cases, the present disclosure provides a modulated effector function (MEF) antibody, wherein the MEF antibody has 1, 2, 3, or 4 reduced interchain disulfide bonds and 2, 4, 6, or 8 biocompatible polymeric moieties (BPMs), respectively; wherein each BPM is covalently attached to each sulfur atom of the cysteine residues of each reduced interchain disulfide bond of the MEF antibody via a cleavable moiety; and wherein the MEF antibody exhibits time-dependent reduction in FcR binding, and thus a corresponding time-dependent reduction in an effector function, relative to that of an equivalent antibody. In some cases, the MEF antibody comprises an effector function increasing modification. In some cases, the effector function enhancing modification increases binding affinity for FcγRI, FcγRIIa, FcγRIIb, FcγRIIIa, FcγRIIIb, or a combination thereof. In some cases, the MEF antibody comprises an IgG antibody.

Reference to an “antibody” as a component of the MEF antibodies of the present disclosure refer to antibodies as described herein, such as therapeutic antibodies. In some cases, an MEF antibody comprises an IgG antibody. In some cases, an MEF antibody is an IgG antibody. In some cases, the IgG antibody in an IgG1 antibody.

In some embodiments, the time-dependent reduction of FcR binding is correlated with the initial presence, and subsequent loss, of the BPMs through cleavage of the corresponding cleavable moiety(ies), for example, in physiological media.

In some embodiments, a MEF antibody as provided herein exhibits decreased binding of the Fc region of the antibody to its cognate Fc receptor relative to an equivalent antibody, as described herein. In some embodiments, the binding to the cognate Fc receptor is decreased by about 10% to about 99%, for example, about 10% to about 50%, about 25% to about 75%, about 50% to about 99%, or any value in between. In some embodiments, the decrease in Fc receptor binding is partially or fully reversed by cleavage of the cleavable moieties.

In some embodiments, a MEF antibody as provided herein binds to the cognate Fc receptor with a binding constant (K_{D}) about 2-fold to about 1,000-fold higher than an equivalent antibody. In some embodiments, the K_{D} for the cognate Fc receptor is about 2-fold to about 10-fold higher, about 5-fold to about 20-fold higher, about 10-fold to about 50-fold higher, about 25-fold to about 100-fold higher, about 50-fold to about 200-fold higher, about 100-fold to about 300-fold higher, about 200-fold to about 400-fold higher, about 300-fold to about 500-fold higher, about 400-fold to about 600-fold higher, about 500-fold to about 700-fold higher, about 600-fold to about 800-fold higher, about 700-fold to about 900-fold higher, about 800-fold to about 1,000-fold higher, or any value in between.

In some embodiments, the increased Fc receptor K_{D} is reduced by cleavage of the cleavable moieties, thereby providing a time-dependent reduction in FcR binding of the MEF antibody. In some embodiments, the time-dependent reduction in FcR binding of the MEF antibody is characterized by an initial reduction in the binding of FcR from at least about 50% to about 90% relative to the equivalent antibody. In some embodiments, the initial reduction of FcR binding is followed by a recovery of the binding as a further characteristic of the time-dependent reduction in FcR binding, wherein the recovery is correlated with BPM loss through non-enzymatic cleavage of the corresponding cleavable moiety(ies) in physiological media, such as vertebrate plasma. In some embodiments, the initial reduction comprises a period of time from the administration of the MEF antibody to a subject (e.g., “0 hours” post-administration) and about 3 hours after administration of the MEF antibody to the subject. For example, about 0 hours to about 2 hours post-administration, about 0 hours to about 1.5 hours post-administration, about 0 hours to about 1 hour post-administration, about 0 hours to about 0.5 hours post-administration, about 0.5 hours to about 2 hours post administration, or about 0.5 hours to 1.5 hours post-administration.

In some embodiments, the plasma half-life of the cleavable moieties is about 3 hours to about 96 hours. For example, the plasma half-life of the cleavable moieties can be about 3 hours to about 12 hours, about 6 hours to about 18 hours, about 12 hours to about 24 hours, about 18 hours to about 36 hours, about 24 hours to about 48 hours, about 36 hours to about 72 hours, about 48 hours to about 96 hours, about 72 to about 120 hours, or any value in between. In specific cases (for example, as outlined in FIG. 13), the cleavable moieties comprise a half-life of between about 60 and about 150 hours, or between about 72 and about 120 hours.

In some embodiments, the K_{D} for the Fc receptor is increased after about 3 hours to about 96 hours. This value can be measured either in vitro or in vivo. In some embodiments, the K_{D} for the Fc receptor is increased when measured in vitro. In some embodiments, the K_{D} for the Fc receptor is increased when measured in vivo. Antibody K_{D} can be measured by, for example, polarization-modulated oblique-incidence reflectivity difference (OI-RD), surface plasmon resonance, interferometry, fluorescence-activated cell sorting (FACS), and by other techniques known in the art. See, e.g., Hearty, et al., Methods Mol. Biol. 2012; 907: 411-442 and Landry, et al., Assay Drug Dev. Tech. 2012; 10: 250-259. In some embodiments, the K_{D} for the Fc receptor can be increased after about 3 hours to about 12 hours, about 6 hours to about 18 hours, about 12 hours to about 24 hours, about 18 hours to about 36 hours, about 24 hours to about 48 hours, about 36 hours to about 72 hours, about 48 hours to about 96 hours, or any value in between.

In some embodiments, the cleavage of the cleavable moieties comprises contacting the cleavable moieties with plasma for a period of time. In some embodiments, the plasma is vertebrate plasma. In some embodiments, the contacting of the cleavable moieties with plasma is in vitro. In some embodiments, the contacting of the cleavable moieties with plasma is in vivo.

In some embodiments, a MEF antibody as provided herein comprises an intact or fully-reduced antibody. The term ‘fully-reduced’ is meant to refer to antibodies in which all inter-chain disulfide linkages have been reduced to provide thiols that can be attached to a cleavable moiety.

A BPM can couple to a range of sites along an antibody. In some cases, a BPM couples to an amino acid residue or post-translational modification of the MEF antibody. In some cases, the BPM couples to a native amino acid residue of the antibody. In some cases, the native amino acid residue is a cysteine residue, a methionine residue, a lysine residue, or a combination thereof. In some cases, the native amino acid residue is a cysteine residue. In some cases, the cysteine residue is reduced from a disulfide bond of the antibody prior to BPM coupling. In some cases, the amino acid residue is provided by means of mutation (e.g., a cysteine residue is provided at a position that typically comprises a valine). In some cases, the post-translational modification comprises glycosylation, nitrosylation, phosphorylation, citrullination, sulfenylation, or a combination thereof. In some cases, the BPM couples to a post-translational modification of the MEF antibody.

In some cases, a BPM is coupled to antibody glycosylation. Coupling a BPM to glycosylation can involve chemically or enzymatically attaching a BPM-modified glycan to the antibody. The BPM-modified glycan can be attached to a glycan, for example with a glycosyltransferase, or an amino acid residue, for example to a serine or threonine with an O—N-acetylgalactosamine-transferase or to an asparagine by an oligosaccharyltransferase. In some cases, coupling a BPM to glycosylation can involve chemically or enzymatically attaching a BPM-to antibody-derived glycosylation. Such coupling can involve oxidation of a terminal glycan monomer to its corresponding dialdehyde, for example with sodium periodate, and coupling a dithiol or diamine of the BPM to the dialdehyde.

In some cases, a BPM is coupled to an antibody-derived nitrosyl group (e.g., post-translationally added nitrosylation). In some cases, the nitrosyl group is coupled to a cysteine, tyrosine, tryptophan, or methionine. The BPM can be electrophilically coupled to a nitrosylated residue following reduction of the nitrosyl group to amine, or, for nitrosylated cysteine, by nucleophilic substitution resulting in disulfide bond formation and nitric oxide displacement. In some cases, a BPM is attached to a citrulline residue of an antibody through a BPM-coupled glyoxal, forming a hydroxyimidazolone adduct with the citrulline urea. In some cases, a BPM is coupled to a sulfenylated residue of an antibody with a 1,3-cycloalkanedione, such as 1,3-cyclohexanedione. In some cases, a BPM is attached to a phosphoryl group of an antibody by forming an adduct between the phosphoryl and a BPM-coupled carbodiimide.

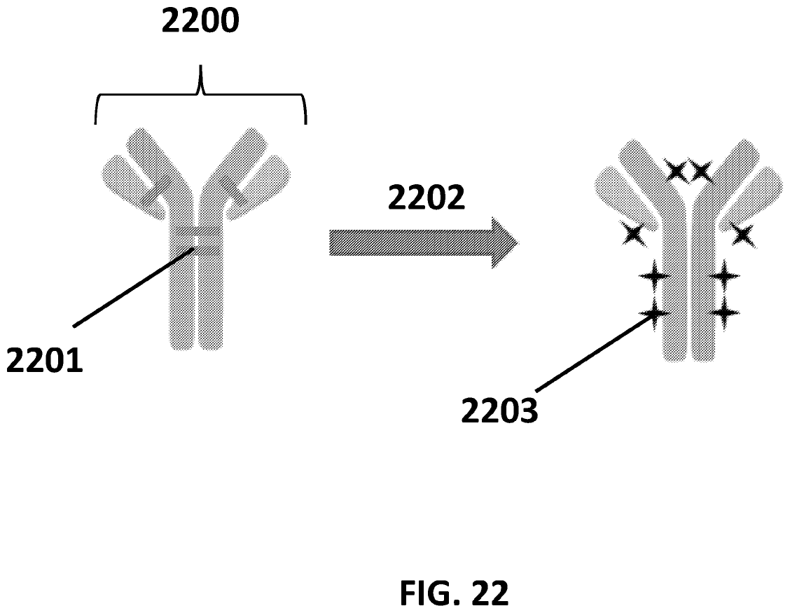

As described herein, each cleavable moiety can be covalently linked to (i) a BPM, and (ii) a sulfur atom of a cysteine residue. In many cases, the cysteine residue is derived from a reduced interchain disulfide bond of a MEF antibody. Each interchain disulfide bond requires a pair of cysteine residues: one on a heavy chain, and the other on either a light chain or a heavy chain. Such a coupling scheme is depicted in FIG. 22, in which interchain disulfide bonds 2201 of an antibody 2200 are reduced 2202 and coupled to BPMs 2203. In some embodiments, after cleavage of a cleavable moiety (releasing the BPM), some part of the cleavable moiety remains attached to the MEF antibody. In some embodiments, cleavage of a cleavable moiety (releasing the BPM), results in a free cysteine thiol group (—SH).

The attachment of the cleavable moiety to a MEF antibody can be via a thioether linkage or disulfide linkage, to a sulfur atom of a cysteine residue of a reduced interchain disulfide bond of the antibody. In some embodiments, the thioether linkage is between a MEF antibody and a succinimide, wherein the cleavable moiety comprises the succinimide. In some embodiments, the thioether linkage is between a MEF antibody and a non-hydrolyzed succinimide, wherein the cleavable moiety comprises the succinimide. In some embodiments, the disulfide linkage is between a MEF antibody the BPM, wherein the cleavable moiety comprises the disulfide linkage. In some embodiments, each cleavable moiety is covalently attached to a sulfur atom of a cysteine residue of a reduced interchain disulfide bond of the MEF antibody through a cleavable disulfide bond, or through a cleavable thioether bond to a non-hydrolyzed succinimide moiety. In some embodiments, each cleavable moiety is covalently attached to a sulfur atom of a cysteine residue of a reduced interchain disulfide bond of the MEF antibody through a cleavable disulfide bond. In some embodiments, each cleavable moiety is covalently attached to a sulfur atom of a cysteine residue of a reduced interchain disulfide bond of the MEF antibody through a cleavable thioether bond to a non-hydrolyzed succinimide moiety.

In some cases, the MEF antibody comprises a ratio of BPMs to Fc regions of between 2 and 20, between 2 and 10, between 2 and 4, between 4 and 12, between 4 and 10, between 6 and 15, between 6 and 10, or between 8 and 15. In some cases, the MEF antibody comprises a ratio of BPMs to fragment antigen-binding (Fab) regions of between 1 and 10, between 1 and 5, between 1 and 3, between 2 and 6, between 2 and 4, between 3 and 8, between 3 and 5, or between 4 and 8.

Without being bound by any theory, when a first cleavable moiety comprising a disulfide linkage is cleaved (releasing a first BPM), the resulting cysteine thiol can preferentially form an interchain disulfide bond with its corresponding cysteine residue, thereby cleaving a second cleavable moiety, and releasing a second BPM. Thus, it is believed that when a first BPM of a pair of cysteine residues of a reduced interchain disulfide bond is released (via cleavage of a disulfide bond), the second BPM, attached to the corresponding cysteine residue via a disulfide bond, will also be released.

In some cases, at least a subset of the BPMs are coupled to cysteine thiols reductively liberated from disulfide bonds. Similarly, without being bound by any theory, it is believed that each pair of BPMs can be bound to the corresponding sulfide residues of a single reduced interchain disulfide bond. Thus, for example, when each cleavable moiety comprises a disulfide linkage, cleavage will occur in a pair-wise fashion, such that the MEF antibody will maintain an even number of BPMs, until all BPMs are lost. In some embodiments, a MEF antibody as described herein comprises 2 BPMs. In some embodiments, a MEF antibody as described herein comprises 4 BPMs. In some embodiments, a MEF antibody as described herein comprises 6 BPMs. In some embodiments, a MEF antibody as described herein comprises 8 BPMs. In some embodiments, a MEF antibody as described herein comprises 10 BPMs.

In contrast, also without being bound by any theory, it is believed that cleavable moieties that do not include a disulfide linkage (such as a thioether linkage to a succinimide) do not necessarily release BPMs in a pair-wise fashion, as do cleavable moieties comprising a disulfide linkage. Accordingly, when the cleavable moiety does not include a disulfide linkage, some embodiments of the antibodies described herein comprise from 1-8 BPMs.

In some embodiments, when each BPM is a polypeptide moiety, each cleavable moiety comprises from 2 to 10 amino acids. Accordingly, in some embodiments, a BPM and a cleavable moiety together comprise from 12 to 60 amino acids.

In some embodiments, each BPM is selected from the group consisting of a polyethylene glycol moiety, a polyketal moiety, a polyglycerol moiety, a polysaccharide moiety, a polysarcosine moiety, a polypeptide moiety, and a polyzwitterionic moiety. In some embodiments, each BPM comprises a monodisperse moiety. In some embodiments, the monodisperse moiety is selected from: a polyethylene glycol moiety, a polyketal moiety, polyglycerol moiety, a polysaccharide moiety, a polysarcosine moiety, a polypeptide moiety, and a polyzwitterionic moiety. In some embodiments, each BPM consists essentially of a monodisperse moiety selected from: a polyethylene glycol moiety, a polyketal moiety, polyglycerol moiety, a polysaccharide moiety, a polysarcosine moiety, a polypeptide moiety, and a polyzwitterionic moiety.

In some embodiments, each BPM comprises a polydisperse moiety. In some embodiments, the polydisperse moiety is selected from: a polyethylene glycol moiety, a polyketal moiety, polyglycerol moiety, a polysaccharide moiety, a polysarcosine moiety, a polypeptide moiety, and a polyzwitterionic moiety. In some embodiments, each BPM consists essentially of a polydisperse moiety selected from: a polyethylene glycol moiety, a polyketal moiety, polyglycerol moiety, a polysaccharide moiety, a polysarcosine moiety, a polypeptide moiety, and a polyzwitterionic moiety.

The average molecular weight of a BPM, as described herein, can be represented by the number-average molecular weight (Mₙ), the weight-average molecular weight (M_{w}), the Z-average molecular weight (M_{z}), and/or the molecular weight at the peak maxima of the molecular weight distribution curve (Mₚ). The average molecular weight of a BPM can be determined by a variety of analytical characterization techniques, such as size-exclusion chromatography (SEC).

In some embodiments, each BPM independently has a weight-average molecular weight of about 100 Daltons to about Daltons 5,000 Daltons. In some embodiments, each BPM independently has a weight-average molecular weight of about 100 Daltons to about 1,000 Daltons, about 600 Daltons to about 1,500 Daltons, about 800 Daltons to about 2,000 Daltons, about 1,000 Daltons to about 2,500 Daltons, about 1,500 Daltons to about 3,000 Daltons, about 2,000 Daltons to about 3,500 Daltons, about 2,500 Daltons to about 4,000 Daltons, about 3,000 Daltons to about 4,500 Daltons, about 3,500 Daltons to about 5,000 Daltons, or any value in between. In some embodiments, each BPM has a molecular weight of between 200 and 1000 Daltons, between 200 and 2000 Daltons, between 300 and 1200 Daltons, between 500 and 1500 Daltons, between 500 and 2500 Daltons, between 500 and 5000 Daltons, between 800 and 3000 Daltons, between 800 and 6000 Daltons, or between 1000 and 8000 Daltons.

The hydrodynamic size of a BPM can influence the behavior of a MEF antibody in a fluid and also influence the pharmacokinetic properties of a MEF antibody. The hydrodynamic size, represented by hydrodynamic radius (Rₕ) or hydrodynamic diameter (Dₕ), can be measured directly or indirectly using analytical characterization techniques such as size-exclusion chromatography (SEC).

In some embodiments, each BPM independently has a hydrodynamic diameter of about 5 nm to about 25 nm. In some embodiments, each BPM independently has a hydrodynamic diameter of about 5 nm to about 10 nm, about 7.5 nm to about 12.5 nm, about 10 nm to about 15 nm, about 12.5 nm to about 17.5 nm, about 15 nm to about 20 nm, about 17.5 nm to about 22.5 nm, about 20 nm to about 25 nm, or any value in between. In some embodiments, each BPM independently has a hydrodynamic diameter of about 15 nm to about 25 nm. In some embodiments, each BPM independently has a hydrodynamic diameter of about 10 nm to about 20 nm. In some embodiments, each BPM independently has a hydrodynamic diameter of about 5 nm to about 15 nm. In some embodiments, each BPM independently has a hydrodynamic diameter of about 5 nm to about 10 nm.

In some embodiments, a plurality of BPMs (e.g., multiple BPMs coupled to an antibody or a plurality of antibodies) is polydisperse. In some embodiments, a plurality of BPMs is monodisperse. In some embodiments, BPMs are discrete, that is, are synthesized in step-wise fashion and not via a polymerization process. Discrete BPMs provide a single molecule with defined and specified chain length.

In some embodiments, a BPM comprises a synthetic polymer, a peptide, an oligosaccharide, a fatty acid, or a combination thereof. In some cases, the BPM comprises PEG, polypropylene glycol, polybutylene glycol, polyglycerin, polyglutamic acid, polylactic acid, polyglycolic acid, polyethylene terephthalate, a derivative thereof, or a combination thereof. In some cases, the BPM comprises PEG, polypropylene glycol, polyglycerin, a derivative thereof, or a combination thereof. In some embodiments, a plurality of BPMs comprises a monodisperse plurality of PEG moieties. In some embodiments, a plurality of BPMs comprises a polydisperse plurality of PEG moieiesy. In some embodiments, each PEG moiety comprises discrete PEGs.

In some embodiments, one terminus of the PEG moiety is directly attached to a MEF antibody via the cleavable moiety, and the other terminus (or termini, in the case of branched PEG moieties) is free and untethered (i.e., not covalently attached). In some embodiments, the free and untethered terminus (or termini) further comprises a cap comprising a suitable functional group such as alkyl, alkyl-carboxylic acid, or alkylamino. In some embodiments, each PEG moiety further comprises a cap selected from the group consisting of —CH₃, —CH₂CH₂CO₂H, —CH₂CH₂NH₂, and combinations thereof.

In some embodiments, when the PEG moiety is branched, each branch comprises an independently selected number of PEG units, e.g., are the same or different chemical moieties, such as having different average molecular weights or number of PEG units.

In some embodiments provided herein, the PEG unit comprises two monomeric polyethylene glycol chains attached to each other via non-PEG elements, which are not part of the repeating PEG structure, such as an amido or urea group.

In some embodiments, each BPM comprises a monodispersed PEG2 to PEG72 moiety. In some embodiments, each BPM comprises a monodispersed PEG4 to PEG48 moiety. In some embodiments, each BPM comprises a monodispersed PEG8 to PEG48 moiety. In some embodiments, each BPM comprises a monodispersed branched PEG20 to PEG76 moiety; and wherein each branch comprises at least a PEG2 unit. In some embodiments, each monodispersed branched PEG20 to PEG76 moiety comprises 2 to 8 branches. In some embodiments, each monodispersed branched PEG20 to PEG76 moiety comprises 2 to 4 branches. In some embodiments, each BPM is a PEG4(PEG8)₃ or a PEG4(PEG24)₃ moiety.

In some embodiments, the PEG moiety comprises one or more linear polyethylene glycol chains each having at least 8 subunits, at least 9 subunits, at least 10 subunits, at least 11 subunits, at least 12 subunits, at least 13 subunits, at least 14 subunits, at least 15 subunits, at least 16 subunits, at least 17 subunits, at least 18 subunits, at least 19 subunits, at least 20 subunits, at least 21 subunits, at least 22 subunits, at least 23 subunits, or at least 24 subunits. In some embodiments, the PEG moiety comprises a combined total of at least 8 subunits, at least 10 subunits, or at least 12 subunits. In some such embodiments, the PEG moiety comprises no more than a combined total of about 72 subunits, preferably no more than a combined total of about 36 subunits. In some embodiments, the PEG comprises about 8 to about 24 subunits (referred to as PEG8 to PEG24).

In some embodiments, the PEG moiety comprises a combined total of from 8 to 72, 8 to 60, 8 to 48, 8 to 36 or 8 to 24 subunits, from 9 to 72, 9 to 60, 9 to 48, 9 to 36 or 9 to 24 subunits, from 10 to 72, 10 to 60, 10 to 48, 10 to 36 or 10 to 24 subunits, from 11 to 72, 11 to 60, 11 to 48, 11 to 36 or 11 to 24 subunits, from 12 to 72, 12 to 60, 12 to 48, 12 to 36 or 12 to 24 subunits, from 13 to 72, 13 to 60, 13 to 48, 13 to 36 or 13 to 24 subunits, from 14 to 72, 14 to 60, 14 to 48, 14 to 36 or 14 to 24 subunits, from 15 to 72, 15 to 60, 15 to 48, 15 to 36 or 15 to 24 subunits, from 16 to 72, 16 to 60, 16 to 48, 16 to 36 or 16 to 24 subunits, from 17 to 72, 17 to 60, 17 to 48, 17 to 36 or 17 to 24 subunits, from 18 to 72, 18 to 60, 18 to 48, 18 to 36 or 18 to 24 subunits, from 19 to 72, 19 to 60, 19 to 48, 19 to 36 or 19 to 24 subunits, from 20 to 72, 20 to 60, 20 to 48, 20 to 36 or 20 to 24 subunits, from 21 to 72, 21 to 60, 21 to 48, 21 to 36 or 21 to 24 subunits, from 22 to 72, 22 to 60, 22 to 48, 22 to 36 or 22 to 24 subunits, from 23 to 72, 23 to 60, 23 to 48, 23 to 36 or 23 to 24 subunits, or from 24 to 72, 24 to 60, 24 to 48, 24 to 36 or 24 subunits.

Illustrative linear PEG moieties include:

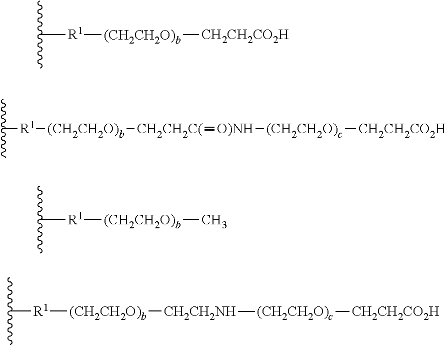

wherein R¹ is a C₂-C₁₂ alkylene, optionally interrupted with one of —NH—C(═O)—, —C(═O)NH—, —NH—, or —O—, and optionally substituted with —CO₂H (as defined for the cleavable moiety); and wherein indicates site of covalent attachment to the cleavable moiety, each subscript b ranges from 2 to 72, and each subscript c ranges from 1 to 72.

In some embodiments, subscript b ranges from 6 to 72. In some embodiments, subscript b ranges from 8 to 72. In some embodiments, subscript b ranges from 10 to 72. In some embodiments, subscript b ranges from 12 to 72. In some embodiments, subscript b ranges from 6 to 24. In some embodiments, subscript b ranges from 8 to 24. In some embodiments, subscript b ranges from 12 to 36. In some embodiments, subscript b ranges from 24 to 48. In some embodiments, subscript b ranges from 36 to 72. In some embodiments, subscript b is about 8, about 12, or about 24.

In some embodiments, subscript c ranges from 1 to 36. In some embodiments, subscript c ranges from 1 to 24. In some embodiments, subscript c ranges from 1 to 12. In some embodiments, subscript c ranges from 1 to 8. In some embodiments, subscript c ranges from 1 to 4. In some embodiments, subscript c is about 1, about 2, or about 2.

In some embodiments, the sum of subscript b and subscript c (b+c) ranges from 6 to 72. In some embodiments, the sum of subscript b and subscript c (b+c) ranges from 8 to 72. In some embodiments, the sum of subscript b and subscript c (b+c) ranges from 10 to 72. In some embodiments, the sum of subscript b and subscript c (b+c) ranges from 12 to 72. In some embodiments, the sum of subscript b and subscript c (b+c) ranges from 6 to 24. In some embodiments, the sum of subscript b and subscript c (b+c) ranges from 8 to 24. In some embodiments, the sum of subscript b and subscript c (b+c) ranges from 12 to 36. In some embodiments, the sum of subscript b and subscript c (b+c) ranges from 24 to 48. In some embodiments, the sum of subscript b and subscript c (b+c) ranges from 36 to 72. In some embodiments, the sum of subscript b and subscript c (b+c) is about 8, about 12, or about 24.

In some embodiments, the PEG moiety is from about 300 Daltons to about 5,000 Daltons; from about 300 Daltons to about 4,000 Daltons; from about 300 Daltons to about 3,000 Daltons; from about 300 daltons to about 2,000 Daltons; from about 300 Daltons to about 1,000 Daltons; or any value in between. In some such aspects, the PEG moiety has at least 8, 10 or 12 subunits. In some embodiments, the PEG has at least 8, 10 or 12 subunits but no more than 72 subunits, preferably no more than 36 subunits.

In some embodiments, apart from the PEG moiety covalently linked to the cleavable moiety, there are no other PEGs present in the antibodies described herein.

In some embodiments, each BPM is a monodisperse polyketal moiety. In some embodiments, each BPM is a polydisperse polyketal moiety. In some embodiments, each polyketal moiety comprises discrete polyketals. In some embodiments, each BPM is a polyketal moiety comprising 2-10 ketal units, 5-10 ketal units, 5-15 ketal units, 10-20 ketal units, or any value in between.

In some embodiments, one terminus of the polyketal moiety is directly attached to a MEF antibody via the cleavable moiety, and the other terminus (or termini, in the case of branched polyketal moieties) is free and untethered (i.e., not covalently attached). In some embodiments, the free and untethered terminus (or termini) further comprises a cap comprising a suitable functional group such as alkyl, alkyl-carboxylic acid, or alkylamino. In some embodiments, each polyketal moiety further comprises a cap selected from the group consisting of —CH₃, —CH₂CH₂CO₂H, —CH₂CH₂NH₂, and combinations thereof.

In some embodiments, when the polyketal moiety is branched, each branch comprises an independently selected number of polyketal units, e.g., are the same or different chemical moieties, such as having different average molecular weights or number of polyketal units.

In some embodiments provided herein, the polyketal unit comprises two monomeric polyketal chains attached to each other via non-polyketal elements, and which are not part of the repeating polyketal structure.

In some embodiments, each BPM is a monodisperse polyglycerol moiety. In some embodiments, each BPM is a polydisperse polyglycerol moiety. In some embodiments, each polyglycerol moiety comprises discrete polyglycerols. In some embodiments, each BPM is a polyglycerol moiety comprising 2-48 glycerol units, 2-6 glycerol units, 2-12 glycerol units, 6-18 glycerol units, 12-24 glycerol units, 18-36 glycerol units, 24-48 glycerol units, or any value in between.

In some embodiments, one terminus of the polyglycerol moiety is directly attached to a MEF antibody via the cleavable moiety, and the other terminus (or termini, in the case of branched polyglycerol moieties) is free and untethered (i.e., not covalently attached). In some embodiments, the free and untethered terminus (or termini) further comprises a cap comprising a suitable functional group such as alkyl, alkyl-carboxylic acid, or alkylamino. In some embodiments, each polyglycerol moiety further comprises a cap selected from the group consisting of —CH₃, —CH₂CH₂CO₂H, —CH₂CH₂NH₂, and combinations thereof.

In some embodiments, when the polyglycerol moiety is branched, each branch comprises an independently selected number of polyglycerol units, e.g., are the same or different chemical moieties, such as having different average molecular weights or number of polyglycerol units.

In some embodiments provided herein, the polyglycerol unit comprises two monomeric polyglycerol chains attached to each other via non-polyglycerol elements, which are not part of the repeating polyglycerol structure.

In some embodiments, each BPM is a monodisperse polysaccharide moiety. In some embodiments, each BPM is a polydisperse polysaccharide moiety. In some embodiments, each polysaccharide moiety comprises discrete polysaccharides. In some embodiments, each BPM is a polysaccharide moiety comprising 2-12 saccharide units, 2-4 saccharide units, 2-6 saccharide units, 2-8 saccharide units, 2-10 saccharide units, 4-8 saccharide units, 6-12 saccharide units, or any value in between. Exemplary saccharide groups include, but are not limited to glucose, fructose, galactose, glucuronic acid, sucrose, lactose, maltose, fructose, trehalose, cellobiose, mannose, fucose, dextran, and any combination thereof.

In some embodiments, one terminus of the polysaccharide moiety is directly attached to a MEF antibody via the cleavable moiety, and the other terminus (or termini, in the case of branched polysaccharide moieties) is free and untethered (i.e., not covalently attached). In some embodiments, one or more hydroxyl groups at the free and untethered terminus (or termini) further comprises a cap comprising a suitable functional group such as alkyl, alkyl-carboxylic acid, or alkylamino. In some embodiments, each polysaccharide moiety further comprises a cap selected from the group consisting of —CH₃, —CH₂CH₂CO₂H, —CH₂CH₂NH₂, and combinations thereof, on one or more hydroxyl groups.

In some embodiments, when the polysaccharide moiety is branched, each branch comprises an independently selected number of polysaccharide units, e.g., are the same or different chemical moieties, such as having different average molecular weights or number of polysaccharide units.

In some embodiments, each BPM is a monodisperse polysarcosine moiety. In some embodiments, each BPM is a polydisperse polysarcosine moiety. In some embodiments, each polysarcosine moiety comprises discrete polysarcosine. In some embodiments, each BPM is a polysarcosine moiety comprising 2-36 sarcosine units, 2-6 sarcosine units, 2-8 sarcosine units, 2-12 sarcosine units, 4-12 sarcosine units, 6-12 sarcosine units, 6-18 sarcosine units, 12-24 sarcosine units, 18-30 sarcosine units, 24-36 sarcosine units, 30-42 sarcosine units, 36-48 sarcosine units, or any value in between.

In some embodiments, each BPM is a monodisperse polypeptide moiety. In some embodiments, each BPM is a polydisperse polypeptide moiety. In some embodiments, each BPM is a polypeptide moiety comprising 3-12 amino acids, 4-10 amino acids, 4-8 amino acids, 5-12 amino acids, 6-15 amino acids, 15-50 amino acids, 15-40 amino acids, 15-30 amino acids, 15-25 amino acids, 15-20 amino acids, 20-30 amino acids, 25-35 amino acids, 30-40 amino acids, 35-45 amino acids, 45-50 amino acids, 25-40 amino acids, or any value in between.

In some embodiments, each BPM is a monodisperse polyzwitterionic moiety. In some embodiments, each BPM is a polydisperse polyzwitterionic moiety. In some embodiments, each polyzwitterionic moiety comprises discrete polyzwitterionic units. See Laschewsky.

In some embodiments, the antibody of the MEF antibodies described herein is a therapeutic antibody. Other than the antibody itself, MEF antibodies as described herein do not contain a therapeutic moiety, i.e., the antibodies do not contain a drug. Likewise, no drug is attached to any cleavable moiety and no drug is attached to any BPM. Moreover, the cleavable moieties, BPMs, and fragments and metabolites thereof, whether attached to the MEF antibody or after cleavage from the MEF antibody, are therapeutically inert, that is, they have no therapeutic effect on a subject. In many instances, the antibodies described herein are not antibody-drug conjugates.

Some embodiments provide a MEF antibody having the structure of Formula (I):

Ab-(S*—X-BPM)ₚ (I)
wherein: each S* is a sulfur atom from a cysteine residue of a reduced interchain disulfide bond of the MEF antibody; each X is a cleavable moiety; each BPM comprises a polyethylene glycol moiety, a polyketal moiety, a polyglycerol moiety, a polysaccharide moiety, a polysarcosine moiety, a polypeptide moiety, or a polyzwitterionic moiety; subscript p is 2, 4, 6, or 8; and
Ab represents the remainder of the antibody.

It will be understood that the antibody of the MEF antibodies described herein is an antibody in residue form such that “Ab” in the structures provided herein incorporates the structure of the MEF antibody.

In some embodiments, subscript p is 2. In some embodiments, subscript p is 4. In some embodiments, subscript p is 6. In some embodiments, subscript p is 8.

In some embodiments, each cleavable moiety is formed from a Michael acceptor moiety. A “Michael acceptor,” as used herein, refers to an α, β-unsaturated electrophile, including, but not limited to, α, β-unsaturated carbonyls (including pyridazinediones), α, β-unsaturated sulfonyls, α, β-unsaturated nitros, α, β-unsaturated nitriles, 5-methylpyrrolones. In some embodiments, a Michael acceptor moiety is formed from a maleimide, for example, which upon the Michael addition forms a succinimide. In some embodiments, each cleavable moiety is formed from a bromomaleimide or a sulfone.

In some embodiments, each cleavable moiety is formed from a sulfur atom of a cysteine thiol from a reduced interchain disulfide bond in a MEF antibody as described herein and a second sulfur atom attached to the BPM, thereby forming a disulfide linkage (—S—S—).

In some embodiments, each cleavable moiety is selected from structures according to Formulas (II) and (III):

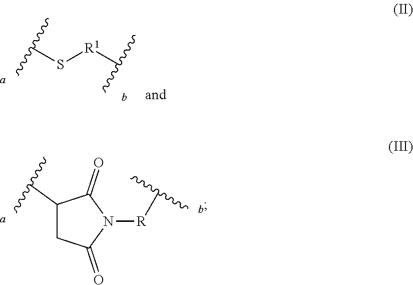

R¹ is a C₂-C₁₂ alkylene, optionally interrupted with one of —NH—C(═O)—, —C(═O)NH—, —NH—, and —O—;
R is absent, or is a C₁-C₁₂ alkylene optionally interrupted with one or two of phenyl, —NH—, —O—, amide, ester, thioester, hydrazone, imine, oxime, sulfate, phosphate ester, or acetal; and R is optionally substituted with 1-3 substituents independently selected from phenyl, oxo, and —CO₂R^{A}; C₃-C₆ cycloalkylene; and phenyl optionally substituted with 1-3 independently selected C₁-C₃ alkoxy; each R^{A} is independently hydrogen or C₁-C₆ alkyl; each R^{1A} is independently hydrogen or C₁-C₆ alkyl; wherein [symbol] (a) represents the covalent attachment to a sulfur atom of the antibody (e.g., the sulfur atom of a cysteine residue of a reduced interchain disulfide bond of the MEF antibody); and
[symbol] (b) represents the covalent attachment to a BPM or the remainder of the cleavable moiety, which retains covalent attachment to a BPM.

In some embodiments, each cleavable moiety is selected from structures according to Formulas (II) and (III):

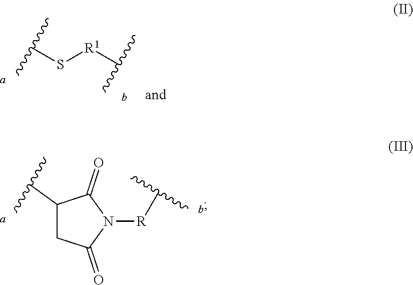

R¹ is a C₂-C₁₂ alkylene, optionally interrupted with one of —NH—C(═O)—, —C(═O)NH—, —NH—, and —O—;
R is absent, or is a C₁-C₁₂ alkylene optionally interrupted with —NH—C(═O)—, —C(═O)NH—, —NH—, —O—, —O—C(═O)—, —C(═O)O—, —S—C(═O)—, —C(═O)S—, —O—C(═O)O—, —C(═NR^{1A}), an acetal, —O(SO₂)O—, —O—[P(═O)(—OH)]O—, —C(═N—OH)—, —C(═N—NH₂)—, and —C(R^{1A})═N—NH—; and R is optionally substituted with 1-3 substituents independently selected from phenyl, oxo, and —CO₂R^{A}; C₃-C₆ cycloalkylene; and phenyl optionally substituted with 1-3 independently selected C₁-C₃ alkoxy; each R^{A} is independently hydrogen or C₁-C₆ alkyl; each R^{1A} is independently hydrogen or C₁-C₆ alkyl; wherein [symbol] (a) represents the covalent attachment to a sulfur atom of the antibody (e.g., the sulfur atom of a cysteine residue of a reduced interchain disulfide bond of the MEF antibody); and
[symbol] (b) represents the covalent attachment to a BPM or the remainder of the cleavable moiety, which retains covalent attachment to a BPM.

In some embodiments, each cleavable moiety has a structure according to either Formula (II) or (III):

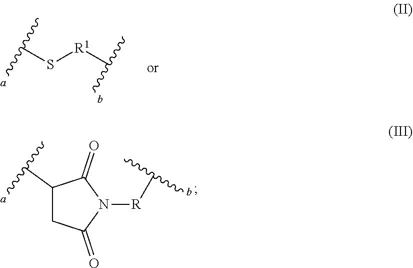

R¹ is a C₂-C₁₂ alkylene, optionally interrupted with one of —NH—C(═O)—, —C(═O)NH—, —NH—, and —O—;
R is absent, or is a C₁-C₁₂ alkylene optionally interrupted with one or two of phenyl, —NH—C(═O)—, —C(═O)NH—, —NH—, —O—, —O—C(═O)—, —C(═O)O—, —S—C(═O)—, —C(═O)S—, —O—C(═O)O—, —C(═NR^{1A}), an acetal, —O(SO₂)O—, —O—[P(═O)(—OH)]O—, —C(═N—OH)—, —C(═N—NH₂)—, and —C(R^{1A})═N—NH—; and R is optionally substituted with 1-3 substituents independently selected from phenyl, oxo, and —CO₂R^{A}; C₃-C₆ cycloalkylene; and phenyl optionally substituted with 1-3 independently selected C₁-C₃ alkoxy; each R^{A} is independently hydrogen or C₁-C₆ alkyl; each R^{1A} is independently hydrogen or C₁-C₆ alkyl; wherein [symbol] (a) represents the covalent attachment to a sulfur atom of the antibody (e.g., a cysteine residue of a reduced interchain disulfide bond of the MEF antibody); and
[symbol] (b) represents the covalent attachment to a BPM or the remainder of the cleavable moiety, which retains covalent attachment to a BPM.

In some embodiments, each cleavable moiety comprises a structure according to Formula (II):

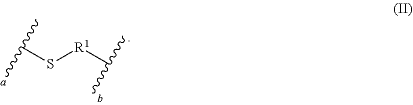

In some embodiments, le is a C₂-C₁₂ alkylene, optionally interrupted with one of —NH—C(═O)—, —C(═O)NH—, —NH—, or —O—, and optionally substituted with —CO₂H. In some embodiments, le is a C₂-C₆ alkylene, optionally interrupted with one of —NH—C(═O)—, —C(═O)NH—, —NH—, or —O—, and optionally substituted with —CO₂H. In some embodiments, le is interrupted at the terminus ( [symbol] (b)). In some embodiments, le is an uninterrupted C₂-C₆ alkylene optionally substituted with —CO₂H. In some embodiments, le is an uninterrupted C₂-C₆ alkylene. In some embodiments, le is an uninterrupted linear C₃-C₆ alkylene, such as n-propyl, n-butyl, n-pentyl, or n-hexyl, optionally substituted with —CO₂H. In some embodiments, le is an uninterrupted linear C₃-C₆ alkylene. In some embodiments, le is an uninterrupted branched C₃-C₆ alkylene, optionally substituted with —CO₂H. In some embodiments, le is substituted with —CO₂H. In some embodiments, le is an uninterrupted branched C₃-C₆ alkylene.

In some embodiments, each cleavable moiety is a structure according to Formula (II):

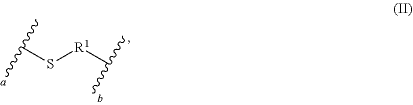

and subscript p is 2. In some embodiments, each cleavable moiety is a structure according to Formula (II):

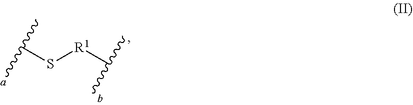

and subscript p is 4. In some embodiments, each cleavable moiety is a structure according to Formula (II):

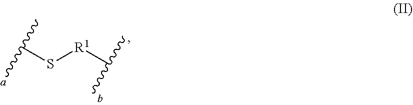

and subscript p is 6. In some embodiments, each cleavable moiety is a structure according to Formula (II):

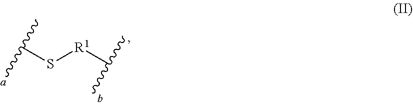

and subscript p is 8.

In some embodiments, each cleavable moiety is selected from one of structures (IIa-IIi) below, wherein [symbol] (a) represents the covalent attachment to a sulfur atom of the antibody (e.g., the sulfur atom of a cysteine residue of a reduced interchain disulfide bond of the MEF antibody); and [symbol] (b) represents the covalent attachment of the cleavable moiety to a BPM.

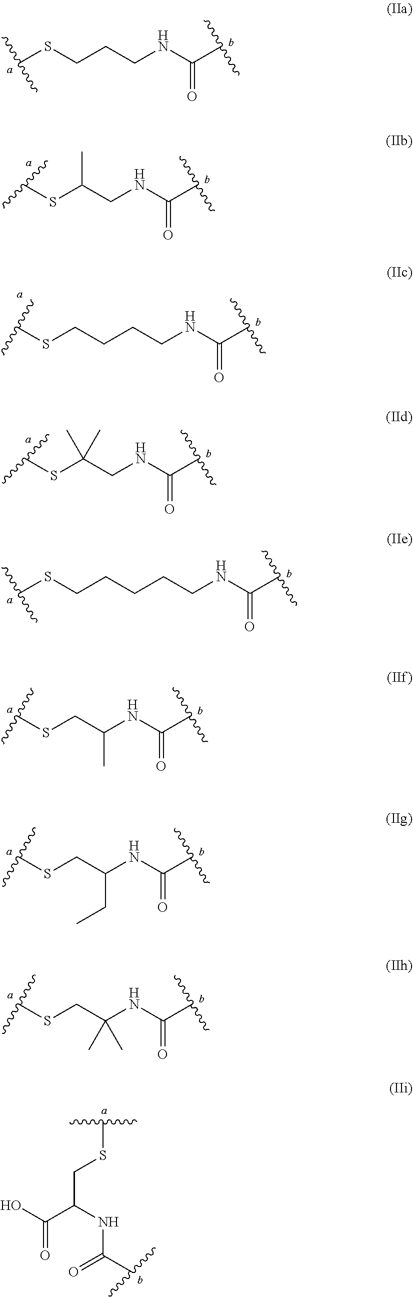

In some embodiments, R¹ is a C₂-C₆ alkylene, interrupted with one of —NH—C(═O)—, —C(═O)NH—, —NH—, or —O—. In some embodiments, R¹ is a C₂-C₆ alkylene, interrupted with —NH—C(═O)— or —C(═O)NH—. In some embodiments, R¹ is -ethylene-NH—C(═O)— or -ethylene-C(═O)NH—. In some embodiments, R¹ is —C₃ alkylene-NH—C(═O)— or —C₃ alkylene-C(═O)NH—. In some embodiments, R¹ is —C₄ alkylene-NH—C(═O)— or —C₄ alkylene-C(═O)NH—.

In some embodiments, each cleavable moiety comprises a structure according to Formula (III):

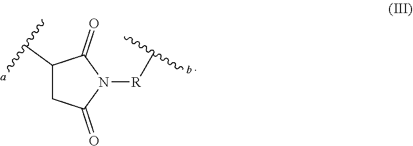

In some embodiments, R is absent. In some embodiments, R is a C₁-C₁₂ alkylene optionally interrupted with one or two of phenyl, —NH—C(═O)—, —C(═O)NH—, —NH—, —O—, —O—C(═O)—, —C(═O)O—, —S—C(═O)—, —C(═O)S—, —O—C(═O)O—, —C(═NR^{1A}), an acetal, a dipeptide, —O(SO₂)O—, —O—[P(═O)(—OH)]O—, —C(═N—OH)—, —C(═N—NH₂)—, and —C(R^{1A})═N—NH—; and R is optionally substituted with 1-3 substituents independently selected from phenyl, oxo, and —CO₂R^{A}; C₃-C₆ cycloalkylene; and phenyl optionally substituted with 1-3 independently selected C₁-C₃ alkoxy.

In some embodiments, R is selected from a C₁-C₁₂ alkylene interrupted with one or two of phenyl, —NH—C(═O)—, —C(═O)NH—, —NH—, —O—, —O—C(═O)—, —C(═O)O—, —S—C(═O)—, —C(═O)S—, —O—C(═O)O—, —C(═NR^{1A}), an acetal, a dipeptide, —O(SO₂)O—, —O—[P(═O)(—OH)]O—, —C(═N—OH)—, —C(═N—NH₂)—, and —C(R^{1A})═N—NH—; and R is optionally substituted with 1-3 substituents independently selected from phenyl, oxo, and —CO₂R^{A}; C₃-C₆ cycloalkylene; and phenyl optionally substituted with 1-3 independently selected C₁-C₃ alkoxy.

In some embodiments, R is selected from a C₁-C₁₂ alkylene optionally interrupted with one or two of phenyl, —NH—C(═O)—, —C(═O)NH—, —NH—, —O—, —O—C(═O)—, —C(═O)O—, —S—C(═O)—, —C(═O)S—, —O—C(═O)O—, —C(═NR^{1A}), an acetal, a dipeptide, —O(SO₂)O—, —O—[P(═O)(—OH)]O—, —C(═N—OH)—, —C(═N—NH₂)—, and —C(R^{1A})═N—NH—; and R is substituted with 1-3 substituents independently selected from phenyl, oxo, and —CO₂R^{A}; C₃-C₆ cycloalkylene; and phenyl optionally substituted with 1-3 independently selected C₁-C₃ alkoxy.

In some embodiments, R is selected from a C₁-C₁₂ alkylene interrupted with one or two of phenyl, —NH—C(═O)—, —C(═O)NH—, —NH—, —O—, —O—C(═O)—, —C(═O)O—, —S—C(═O)—, —C(═O)S—, —O—C(═O)O—, —C(═NR^{1A}), an acetal, a dipeptide, —O(SO₂)O—, —O—[P(═O)(—OH)]O—, —C(═N—OH)—, —C(═N—NH₂)—, and —C(R^{1A})═N—NH—; and R is substituted with 1-3 substituents independently selected from phenyl, oxo, and —CO₂R^{A}; C₃-C₆ cycloalkylene; and phenyl optionally substituted with 1-3 independently selected C₁-C₃ alkoxy.

In some embodiments, R is selected from a C₁-C₁₂ alkylene interrupted with phenyl, —NH—C(═O)—, —C(═O)NH—, —NH—, —O—, —O—C(═O)—, —C(═O)O—, —S—C(═O)—, —C(═O)S—, —O—C(═O)O—, —C(═NR^{1A}), an acetal, a dipeptide, —O(SO₂)O—, —O—[P(═O)(—OH)]O—, —C(═N—OH)—, —C(═N—NH₂)—, or —C(R^{1A})═N—NH—.

In some embodiments, R is selected from a C₁-C₁₂ alkylene interrupted with two groups independently selected from phenyl, —NH—C(═O)—, —C(═O)NH—, —NH—, —O—, —O—C(═O)—, —C(═O)O—, —S—C(═O)—, —C(═O)S—, —O—C(═O)O—, —C(═NR^{1A}), an acetal, a dipeptide, —O(SO₂)O—, —O—[P(═O)(—OH)]O—, —C(═N—OH)—, —C(═N—NH₂)—, and —C(R^{1A})═N—NH—. In some embodiments, R is selected from a C₁-C₁₂ alkylene interrupted with two groups independently selected from phenyl, —NH—C(═O)—, —C(═O)NH—, —NH—, —O—, —O—C(═O)—, —C(═O)O—, —S—C(═O)—, —C(═O)S—, —O—C(═O)O—, —C(═NR^{1A}), an acetal, —O(SO₂)O—, —O—[P(═O)(—OH)]O—, —C(═N—OH)—, —C(═N—NH₂)—, and —C(R^{1A})═N—NH—; and R is substituted with 1 or 2 oxo groups.

In some embodiments, R is a C₁-C₁₂ alkylene optionally substituted with 1-3 substituents independently selected from phenyl and —CO₂R^{A}. In some embodiments, R is an unsubstituted C₁-C₁₂ alkylene. In some embodiments, R is a C₁-C₁₂ alkylene substituted with 1-3 substituents independently selected from phenyl and —CO₂R^{A}. In some embodiments, R is a C₁-C₁₂ alkylene substituted with two or three phenyl groups. In some embodiments, R is a C₁-C₁₂ alkylene substituted with two phenyl groups and —CO₂R^{A}.

In some embodiments, the C₁-C₁₂ alkylene is a C₂-C₆ alkylene. In some embodiments, the C₁-C₁₂ alkylene is a C₂ alkylene, a C₃ alkylene, a C₄ alkylene, a C₅ alkylene, a C₆ alkylene, a C₇ alkylene, or a C₈ alkylene. In some embodiments, the C₁-C₁₂ alkylene is a C₂ alkylene, a C₃ alkylene, or a C₄ alkylene. In some embodiments, the alkylene is branched, such as 2-propyl, 2-hexyl, 3-pentanyl, or t-butyl. In some embodiments, the alkylene is straight chained, such as methylene, ethylene, propylene, butylene, pentylene, or hexylene.

In some embodiments, R is a C₃-C₆ cycloalkylene, such as cyclopropylene, cyclobutylene, cyclopentylene, or cyclohexylene.

In some embodiments, R is a phenyl optionally substituted with 1-3 independently selected C₁-C₃ alkoxy. In some embodiments, R is an unsubstituted phenyl. In some embodiments, R is a phenyl substituted with 1-3 independently selected C₁-C₃ alkoxy. In some embodiments, R is 2,4,6-trimethoxyphenyl.

In some embodiments, each R^{A} is hydrogen. In some embodiments, each R^{A} is C₁-C₆ alkyl. In some embodiments, one or more R^{A} is hydrogen and the remaining R^{A} are C₁-C₆ alkyl. In some embodiments, one or more R^{A} is C₁-C₆ alkyl and the remaining R^{A} are hydrogen.

In some embodiments, each R^{1A} is hydrogen. In some embodiments, each R^{1A} is C₁-C₆ alkyl. In some embodiments, one or more R^{1A} is hydrogen and the remaining R^{1A} are C₁-C₆ alkyl. In some embodiments, one or more R^{1A} is C₁-C₆ alkyl and the remaining R^{1A} are hydrogen.

In some embodiments, each cleavable moiety has a structure according to Formula (III):

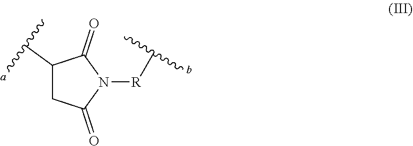

and further comprises a functional group selected from ester, carbonate, amide, imine, an acetal, a dipeptide, sulfate, phosphate ester, oxime, thioester, and hydrazone, as described herein. In some embodiments, the aforementioned functional group is capable of cleavage by hydrolysis, resulting in loss of the corresponding BPM, wherein the aforementioned functional group comprises the remainder of the cleavable moiety. In some embodiments, each cleavable moiety further comprises a hydrazone.

In some embodiments, each BPM and cleavable moiety, together with a sulfur atom of the antibody (e.g., the sulfur atom of a cysteine residue of a reduced interchain disulfide bond of a MEF antibody as described herein), has a structure according to any one of Formulas (IIj-IIn):

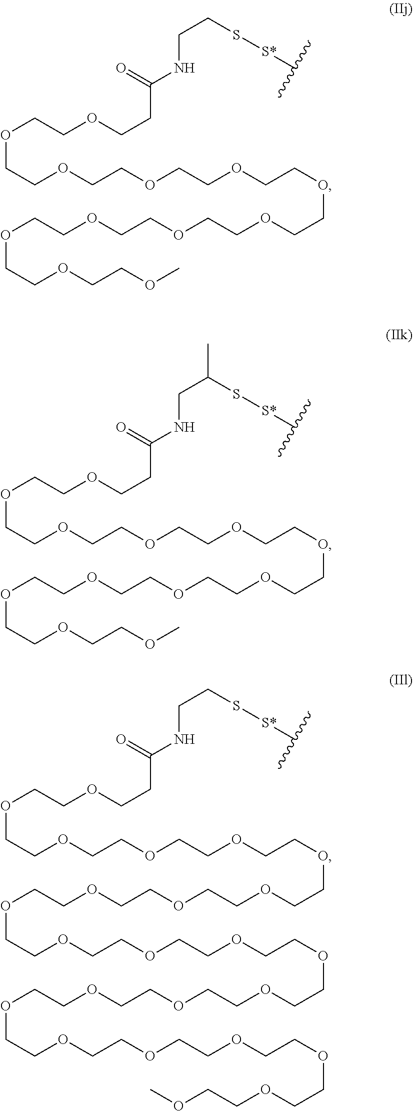

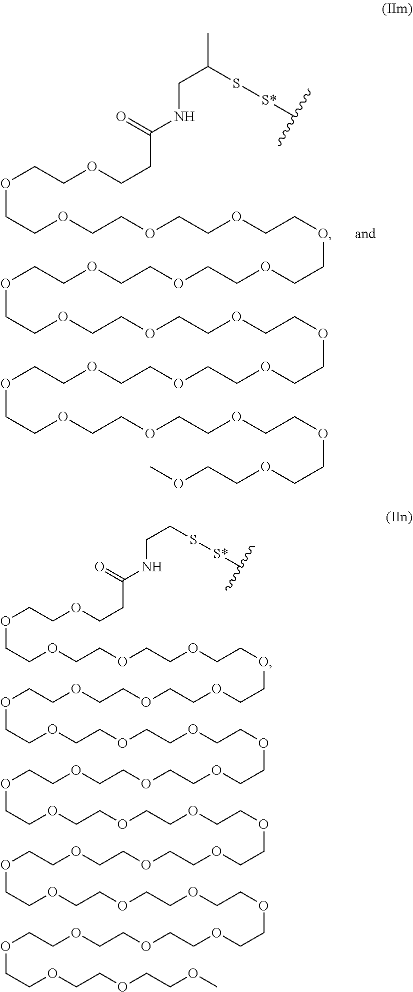

wherein S* is a sulfur atom of the antibody (e.g., the sulfur atom from the cysteine residue of the reduced interchain disulfide bond of the MEF antibody); and wherein [symbol] indicates covalent attachment to the remainder of the MEF antibody.

In some embodiments, each BPM and cleavable moiety, together with a sulfur atom of the antibody (e.g., the sulfur atom of a cysteine residue of a reduced interchain disulfide bond of the MEF antibody), has a structure of any one of Formulas (IIIa)-(IIIg):

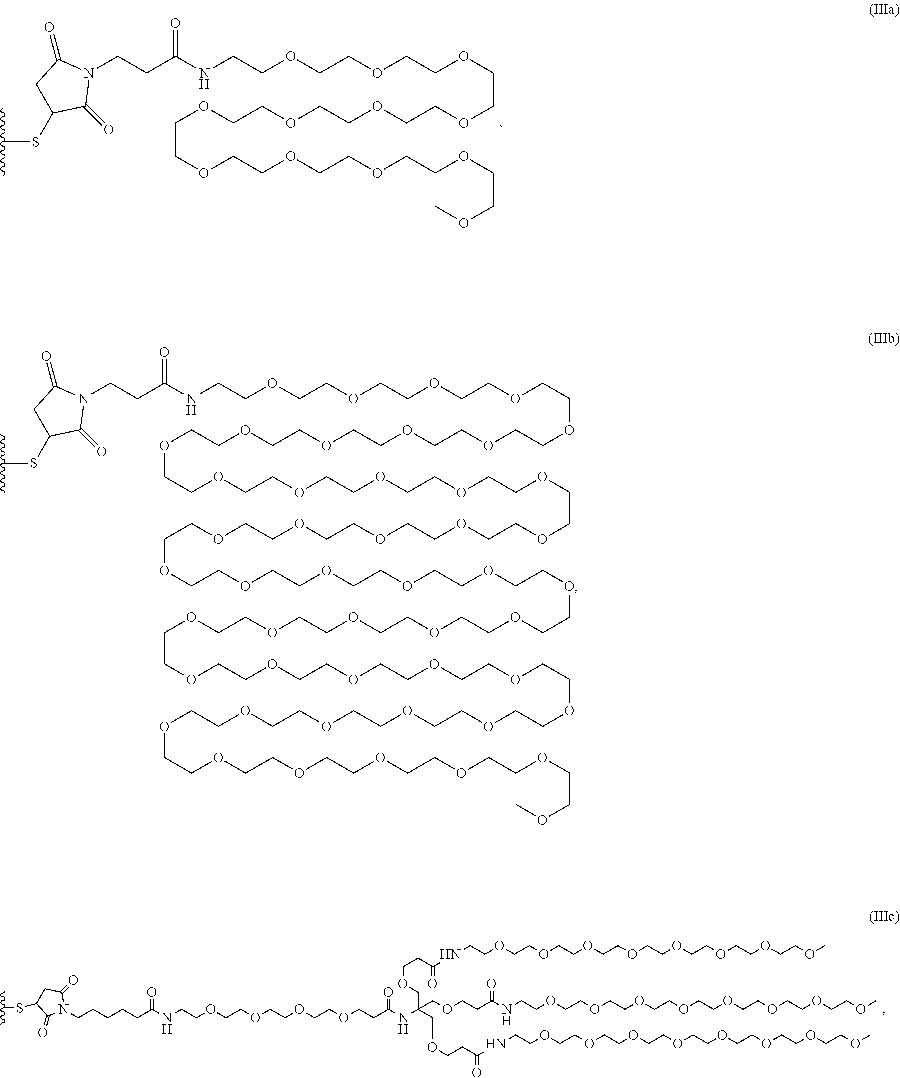 [SMILES: *SC1CC(=O)N(CCC(=O)NCCOCCOCCOCCOCCOCCOCCOCCOCCOCCOCCOCCOC)C1=O ; *SC1CC(=O)N(CCC(=O)NCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOC)C1=O ; *SC1CC(=O)N(CCCCCC(=O)NCCOCCOCCOCCOCCC(=O)NC(COCCC(=O)NCCOCCOCCOCCOCCOCCOCCOCCOC)(COCCC(=O)NCCOCCOCCOCCOCCOCCOCCOCCOC)COCCC(=O)NCCOCCOCCOCCOCCOCCOCCOCCOC)C1=O]

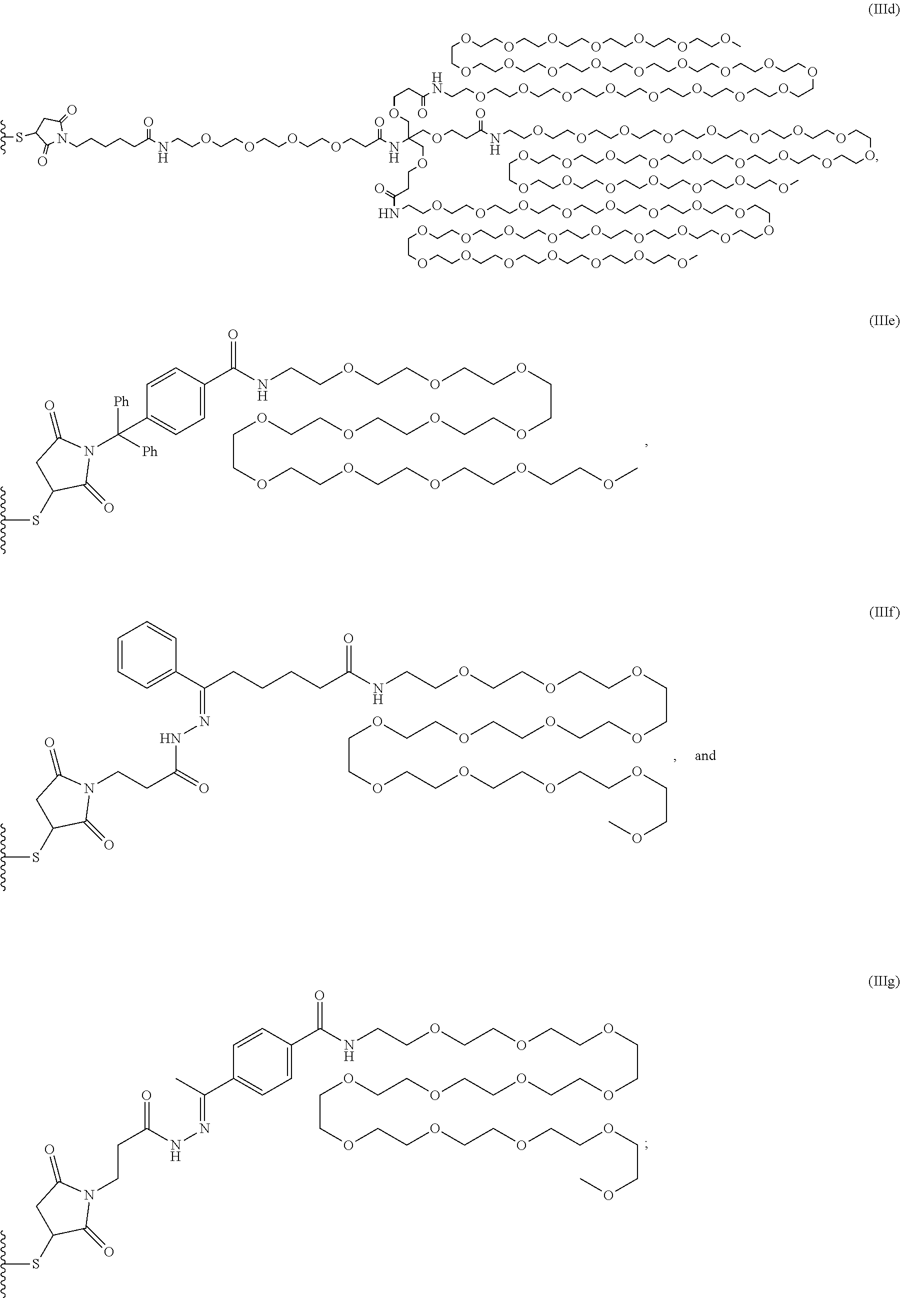

wherein [symbol] indicates covalent attachment to a sulfur atom of the antibody (e.g., the remainder of the cysteine residue of a reduced interchain disulfide bond of the MEF antibody).

In some embodiments, each cleavable moiety comprises a structure of any one of Formulas (IIIh-IIIk):

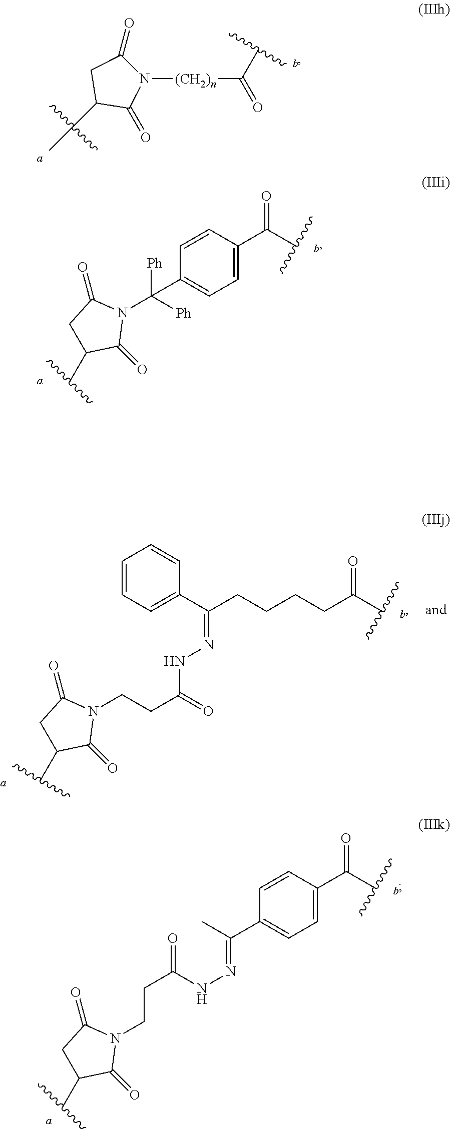

wherein subscript n is an integer ranging from 2 to 8;
[symbol] (a) represents the covalent attachment to a sulfur atom of the antibody (e.g., the sulfur atom of a cysteine residue of a reduced interchain disulfide bond of the MEF antibody); and [symbol] (b) represents the covalent attachment of the cleavable moiety to a BPM.

In some embodiments, each cleavable moiety comprises a structure according to Formula (IIIl):

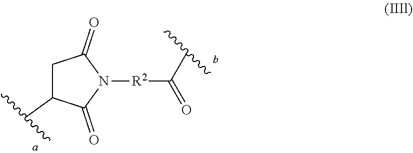

wherein:
R² is C₁-C₁₅ alkyl optionally substituted with one or more instances of hydroxyl, halogen, —CN, C₁-C₆ alkyl, C₁-C₆ alkenyl, C₁-C₆ alkynyl, C₁-C₆ alkoxyl, C₁-C₆ thioalkoxy, —C₁-C₆ cycloalkyl, —NR³R⁴, —C(═O)—R³, —C(═O)—OR⁵, PEG2-PEG72, or a combination thereof;
R³ and R⁴ are each independently selected from the group consisting of H,
[symbol] (a) represents the covalent attachment to a sulfur atom of the antibody (e.g., the sulfur atom of a cysteine residue of a reduced interchain disulfide bond of the MEF antibody); and
[symbol] (b) represents the covalent attachment to a BPM. In some embodiments, R² is C₁-C₁₅ alkyl optionally substituted with one ore more instances of hydroxyl, halogen, —CN, C₁-C₆ alkyl, C₁-C₆ alkoxyl, or a combination thereof. In some embodiments, R² is C₁-C₁₂ alkyl optionally substituted with one more instances of hydroxyl, halogen, —CN, C₁-C₃ alkyl, C₁-C₃ alkoxyl, or a combination thereof. In some embodiments, R² is C₁-C₁₂ alkyl optionally substituted with one more instances of hydroxyl, halogen, or —C₁-C₃ alkyl.

In some embodiments, each cleavable moiety has a structure according to Formula (IIIh):

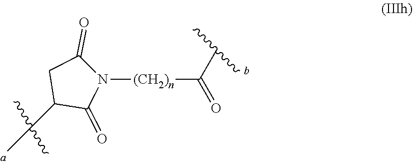

wherein subscript n is an integer ranging from 2 to 8; and
   wherein [symbol] (a) represents the covalent attachment to a sulfur atom of the antibody (e.g., the sulfur atom of a cysteine residue of a reduced interchain disulfide bond of the MEF antibody); and [symbol] (b) represents the covalent attachment of the cleavable moiety to a BPM.

In some embodiments, each BPM has a structure according to Formula (IVa):

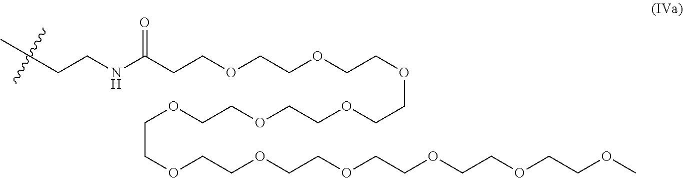

wherein [symbol] represents the covalent attachment to a cleavable moiety.

In some embodiments, each —X-BPM moiety has a structure according to Formula (Mb):

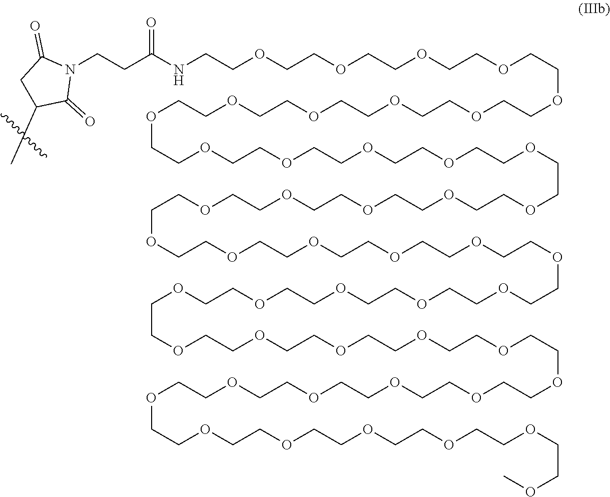

wherein [symbol] represents the covalent attachment to S*.

In some embodiments, each —X-BPM moiety has a structure according to Formula (IIIm):

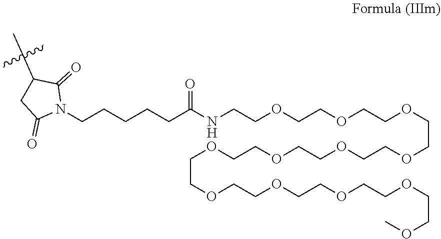

wherein [symbol] represents the covalent attachment to S*.

In some embodiments, a MEF antibody as described herein comprises BPMs covalently attached primarily in the hinge region of the antibody, for example, greater than 50% of the BPMs are covalently attached in the hinge region, greater than 75% of the BPMs are covalently attached in the hinge region, or greater than 90% of the BPMs are covalently attached in the hinge region. In some embodiments, a MEF antibody as described herein comprises BPMs covalently attached primarily in the Fab region of the antibody, for example, greater than 50% of the BPMs are covalently attached in the Fab region, greater than 75% of the BPMs are covalently attached in the Fab region, or greater than 90% of the BPMs are covalently attached in the Fab region. In some embodiments, a MEF antibody as described herein comprises BPMs covalently attached only in the hinge region of the antibody. In some embodiments, a MEF antibody as described herein comprises BPMs covalently attached only in the Fab region of the antibody.

In some embodiments, a MEF antibody as described herein is an IgG antibody. In some embodiments, a MEF antibody as described herein is an IgG₁ antibody. In some embodiments, a MEF antibody as described herein is an IgG₂ antibody. In some embodiments, a MEF antibody as described herein is an IgG₃ antibody. In some embodiments, a MEF antibody as described herein is an IgG₄ antibody. In some embodiments, a MEF antibody as described herein is a monospecific antibody. In some embodiments, a MEF antibody as described herein is a multispecific (e.g., bispecific) antibody. In some embodiments, a MEF antibody as described herein is a polyclonal antibody. In some embodiments, a MEF antibody as described herein is a monoclonal antibody. In some embodiments, the monoclonal antibody is a chimeric antibody. In some embodiments, the monoclonal antibody is a humanized antibody. In some embodiments, the MEF antibodies described herein are present in salt form. In some embodiments, the MEF antibodies described herein are present in pharmaceutically acceptable salt form.

### Antibody Targets

Various aspects of the present disclosure provide MEF antibodies configured to bind to a range of target species. In some embodiments, the MEF antibody binds to a cancer cell. In some embodiments, the MEF antibody binds to a cancer cell antigen which is on the surface of a cancer cell. In some embodiments, the MEF antibody binds to an immune cell. In some embodiments, the MEF antibody binds to an immune cell antigen which is on the surface of an immune cell. In some embodiments, the antibodies described herein are directed against a cancer cell antigen. In some embodiments, the antibodies are directed against a bacteria-related antigen. In some embodiments, the antibodies are directed against a virus-related antigen. In some embodiments, the antibodies are directed against an immune cell antigen.

In some embodiments, an antibody includes a functionally active fragment, derivative or analog of an antibody that immunospecifically binds to target cells (e.g., cancer cell antigens, viral antigens, or microbial antigens) or other antibodies bound to tumor cells or matrix. In this regard, “functionally active” means that the fragment, derivative or analog is able to immunospecifically binds to target cells. The antigen specificity of antibodies is defined by the amino acid sequence of their complementarity-determining region (CDR). To determine which CDR sequences bind the antigen, synthetic peptides containing the CDR sequences are typically used in binding assays with the antigen by any binding assay method known in the art (e.g., the BIA core assay) (See, e.g., Kabat et al., 1991, Sequences of Proteins of Immunological Interest, Fifth Edition, National Institute of Health, Bethesda, Md; Kabat E et al., 1980, J. Immunology 125(3):961-969).

Additionally, recombinant antibodies, such as chimeric and humanized monoclonal antibodies, comprising both human and non-human portions, which are typically obtained using standard recombinant DNA techniques, are useful antibodies. A chimeric antibody is a molecule in which different portions are derived from different animal species, such as for example, those having a variable region derived from a murine monoclonal and human immunoglobulin constant regions. See, e.g., U.S. Pat. Nos. 4,816,567; and 4,816,397, which are incorporated herein by reference in their entirety. Humanized antibodies are antibody molecules from non-human species having one or more complementarity determining regions (CDRs) from the non-human species and a framework region from a human immunoglobulin molecule. (See, e.g., U.S. Pat. No. 5,585,089, which is incorporated herein by reference in its entirety.) Such chimeric and humanized monoclonal antibodies can be produced by recombinant DNA techniques known in the art, for example using methods described in Berter et al., 1988, Science 240:1041-1043; Liu et al., 1987, Proc. Natl. Acad. Sci. USA 84:3439-3443; Liu et al., 1987, J. Immunol. 139:3521-3526; Sun et al., 1987, Proc. Natl. Acad. Sci. USA 84:214-218; Nishimura et al., 1987, Cancer. Res. 47:999-1005; Wood et al., 1985, Nature 314:446-449; and Shaw et al., 1988, J. Natl. Cancer Inst. 80:1553-1559; Morrison, 1985, Science 229:1202-1207; Oi et al., 1986, BioTechniques 4:214; U.S. Pat. No. 5,225,539; Jones et al., 1986, Nature 321:552-525; Verhoeyan et al., 1988, Science 239:1534; and Beidler et al., 1988, J. Immunol. 141:4053-4060; each of which is incorporated herein by reference in its entirety.

Useful polyclonal antibodies are heterogeneous populations of antibody molecules derived from the sera of immunized animals. Useful monoclonal antibodies are homogeneous populations of antibodies to a particular antigenic determinant (e.g., a cancer cell antigen, a viral antigen, a microbial antigen, a protein, a peptide, a carbohydrate, a chemical, nucleic acid, or fragments thereof). A monoclonal antibody (mAb) to an antigen-of-interest can be prepared by using any technique known in the art which provides for the production of antibody molecules by continuous cell lines in culture.

Useful monoclonal antibodies include, but are not limited to, human monoclonal antibodies, humanized monoclonal antibodies, or chimeric human-mouse (or other species) monoclonal antibodies. The antibodies include full-length antibodies and antigen binding fragments thereof. Human monoclonal antibodies may be made by any of numerous techniques known in the art (e.g., Teng et al., 1983, Proc. Natl. Acad. Sci. USA. 80:7308-7312; Kozbor et al., 1983, Immunology Today 4:72-79; and Olsson et al., 1982, Meth. Enzymol. 92:3-16).

In some embodiments, an antibody as described herein is a completely human antibody. In some embodiments, an antibody as described herein is produced using transgenic mice that are incapable of expressing endogenous immunoglobulin heavy and light chains genes, but which are capable of expressing human heavy and light chain genes.

Antibodies immunospecific for a cancer cell antigen are available commercially or produced by any method known to one of skill in the art such as, e.g., chemical synthesis or recombinant expression techniques. The nucleotide sequence encoding antibodies immunospecific for a cancer cell antigen are obtainable, e.g., from the GenBank database or a database like it, the literature publications, or by routine cloning and sequencing.

The MEF antibody can contain a modification which increases its effector function. Combining time-dependent effector function inhibition (e.g., site-selective PEGylation as disclosed in embodiments herein) with effector function enhancing modifications can lead to controllable, high potency treatments. As an antibody disclosed herein can localize to a target site, such as a particular type of cancer cell, effector function enhancing modifications can intensify localized immune responses during treatment, while the time-dependent effector function inhibition can prevent immune overactivation and deleterious systemic effects.

In some cases, the effector function increasing modification comprises a change in glycosylation. In many antibodies (e.g., many IgG antibodies), Fc region glycosylation affects binding to a wide range of proteins which can alter systemic clearance and immune activation, including FcRs (e.g., FcγRs), FcRns, and complement proteins. In many cases, altering glycosylation affects not only the strength of antibody-receptor interactions, but also the types of receptors which preferentially bind to the antibody. In some embodiments, a MEF antibody as described herein comprises one or more fucosyl groups. In some embodiments, a MEF antibody as described herein is afucosylated. In some embodiments, each BPM comprises one or more fucosyl groups, but the MEF antibody is afucosylated (i.e., there are no fucosyl groups directly attached to the MEF antibody). In some cases, a MEF antibody as described herein comprises one or more galactose groups. In some cases, the MEF antibody does not comprise a galactose group. In some cases, the MEF antibody is sialylated (comprises a sialic acid moiety). In some cases, the MEF antibody is not sialylated.

In some embodiments, an antibody as described herein comprises one or more mutations in the Fc region (for example, in each heavy chain of the Fc region); wherein the MEF antibody having one or more mutations has higher effector function relative to an equivalent antibody without the one or more mutations. In some embodiments, an antibody as described herein is an IgG₁ antibody; and the one or more mutations in the Fc region are selected from the group consisting of S298A, E333A, K334A, S239D, 1332E, G236A, S239E, A330L, G236A, L234Y, G236W, S296A, F243, R292P, Y300L, V305L, and P396L. In some embodiments, the one or more mutations are selected from: S298A/E333A/K334A, S239D/I332E, G236A/S239E/A330L/1332E, S239D/I332E, L234Y/G236W/S296A, G236A, F243, R292P, Y300L, V305L and P396L. In some embodiments, the one or more mutations is one mutation. In some embodiments, the one or more mutations are two mutations. In some embodiments, the one or more mutations are three mutations. In some embodiments, the one or more mutations are four or more mutations. In some embodiments, the MEF antibody comprising one or more mutations in the Fc region, as described herein, is an afucosylated antibody.

In some embodiments, an antibody as described herein is a known antibody for the treatment of cancer (e.g., an antibody approved by the FDA and/or EMA). Antibodies immunospecific for a cancer cell antigen are obtainable commercially or produced by any method known to one of skill in the art such as, e.g., recombinant expression techniques. The nucleotide sequence encoding antibodies immunospecific for a cancer cell antigen are obtainable, e.g., from the GenBank database or a database like it, the literature publications, or by routine cloning and sequencing.

In some embodiments, the antibodies described herein for the treatment of an autoimmune disorder are used in accordance with the compositions and methods described herein. Antibodies immunospecific for an antigen of a cell that is responsible for producing autoimmune antibodies are obtainable if not commercially or otherwise available by any method known to one of skill in the art such as, e.g., chemical synthesis or recombinant expression techniques.

In some embodiments, the antibodies described herein are to a receptor or a receptor complex expressed on an activated lymphocyte. The receptor or receptor complex can comprise an immunoglobulin gene superfamily member, a TNF receptor superfamily member, an integrin, a cytokine receptor, a chemokine receptor, a major histocompatibility protein, a lectin, or a complement control protein.

Exemplary antigens are provided below. Exemplary antibodies that bind the indicated antigen are shown in parentheses.

In some embodiments, the antigen is a tumor-associated antigen. In some embodiments, the tumor-associated antigen is a transmembrane protein. For example, the following antigens are transmembrane proteins: ANTXR1, BAFF-R, CA9 (exemplary antibodies include girentuximab), CD147 (exemplary antibodies include gavilimomab and metuzumab), CD19, CD20 (exemplary antibodies include divozilimab and ibritumomab tiuxetan), CD274 also known as PD-L1 (exemplary antibodies include adebrelimab, atezolizumab, garivulimab, durvalumab, and avelumab), CD30 (exemplary antibodies include iratumumab and brentuximab), CD33 (exemplary antibodies include lintuzumab), CD352, CD45 (exemplary antibodies include apamistamab), CD47 (exemplary antibodies include letaplimab and magrolimab), CLPTM1L, DPP4, EGFR, ERVMER34-1, FASL, FSHR, FZD5, FZD8, GUCY2C (exemplary antibodies include indusatumab), IFNAR1 (exemplary antibodies include faralimomab), IFNAR2, LMP2, MLANA, SITZ, TLR2/4/1 (exemplary antibodies include tomaralimab), TM4SF5, TMEM132A, TMEM40, UPK1B, VEGF, and VEFGR2 (exemplary antibodies include gentuximab).

In some embodiments, the tumor-associated antigen is a transmembrane transport protein. For example, the following antigens are transmembrane transport proteins: ASCT2 (exemplary antibodies include idactamab), MFSD13A, Mincle, NOX1, SLC10A2, SLC12A2, SLC17A2, SLC38A1, SLC39A5, SLC39A6 also known as LIV1 (exemplary antibodies include ladiratuzumab), SLC44A4, SLC6A15, SLC6A6, SLC7A11, and SLC7A5.

In some embodiments, the tumor-associated antigen is a transmembrane or membrane-associated glycoprotein. For example, the following antigens are transmembrane or membrane-associated glycoproteins: CA-125, CA19-9, CAMPATH-1 (exemplary antibodies include alemtuzumab), carcinoembryonic antigen (exemplary antibodies include arcitumomab, cergutuzumab, amunaleukin, and labetuzumab), CD112, CD155, CD24, CD247, CD37 (exemplary antibodies include lilotomab), CD38 (exemplary antibodies include felzartamab), CD3D, CD3E (exemplary antibodies include foralumab and teplizumab), CD3G, CD96, CDCP1, CDH17, CDH3, CDH6, CEACAM1, CEACAM6, CLDN1, CLDN16, CLDN18.1 (exemplary antibodies include zolbetuximab), CLDN18.2 (exemplary antibodies include zolbetuximab), CLDN19, CLDN2, CLEC12A (exemplary antibodies include tepoditamab), DPEP1, DPEP3, DSG2, endosialin (exemplary antibodies include ontuxizumab), ENPP1, EPCAM (exemplary antibodies include adecatumumab), FN, FN1, Gp100, GPA33, gpNMB (exemplary antibodies include glembatumumab), ICAM1, L1CAM, LAMP1, MELTF also known as CD228, NCAM1, Nectin-4 (exemplary antibodies include enfortumab), PDPN, PMSA, PROM1, PSCA, PSMA, Siglecs 1-16, SIRPa, SIRPg, TACSTD2, TAG-72, Tenascin, Tissue Factor also known as TF (exemplary antibodies include tisotumab), and ULBP1/2/3/4/5/6.

In some embodiments, the tumor-associated antigen is a transmembrane or membrane-associated receptor kinase. For example, the following antigens are transmembrane or membrane-associated receptor kinases: ALK, Axl (exemplary antibodies include tilvestamab), BMPR2, DCLK1, DDR1, EPHA receptors, EPHA2, ERBB2 also known as HER2 (exemplary antibodies include trastuzumab, bevacizumab, pertuzumab, and margetuximab), ERBB3, FLT3, PDGFR-B (exemplary antibodies include rinucumab), PTK7 (exemplary antibodies include cofetuzumab), RET, ROR1 (exemplary antibodies include cirmtuzumab), ROR2, ROS1, and Tie3.

In some embodiments, the tumor-associated antigen is a membrane-associated or membrane-localized protein. For example, the following antigens are membrane-associated or membrane-localized proteins: ALPP, ALPPL2, ANXA1, FOLR1 (exemplary antibodies include farletuzumab), IL13Ra2, IL1RAP (exemplary antibodies include nidanilimab), NT5E, OX40, Ras mutant, RGS5, RhoC, SLAMF7 (exemplary antibodies include elotuzumab), and VSIR.

In some embodiments, the tumor-associated antigen is a transmembrane G-protein coupled receptor (GPCR). For example, the following antigens are GPCRs: CALCR, CD97, GPR87, and KISS1R.

In some embodiments, the tumor-associated antigen is cell-surface-associated or a cell-surface receptor. For example, the following antigens are cell-surface-associated and/or cell-surface receptors: B7-DC, B-cell maturation antigen (BCMA), CD137, CD 244, CD3 (exemplary antibodies include otelixizumab and visilizumab), CD48, CD5 (exemplary antibodies include zolimomab aritox), CD70 (exemplary antibodies include cusatuzumab and vorsetuzumab), CD74 (exemplary antibodies include milatuzumab), CD79A, CD-262 (exemplary antibodies include tigatuzumab), DR4 (exemplary antibodies include mapatumumab), FAS, FGFR1, FGFR2 (exemplary antibodies include aprutumab), FGFR3 (exemplary antibodies include vofatamab), FGFR4, GITR (exemplary antibodies include ragifilimab), Gpc3 (exemplary antibodies include ragifilimab), HAVCR2, HLA-E, HLA-F, HLA-G, LAG-3 (exemplary antibodies include encelimab), LY6G6D, LY9, MICA, MICB, MSLN, MUC1, MUC5AC, NY-ESO-1, OY-TES1, PVRIG, Sialyl-Thomsen-Nouveau Antigen, Sperm protein 17, TNFRSF12, and uPAR.

In some embodiments, the tumor-associated antigen is a chemokine receptor or cytokine receptor. For example, the following antigens are chemokine receptors or cytokine receptors: CD115 (exemplary antibodies include axatilimab, cabiralizumab, and emactuzumab), CD123, CXCR 4 (exemplary antibodies include ulocuplumab), IL-21R, and IL-5R (exemplary antibodies include benralizumab).

In some embodiments, the tumor-associated antigen is a co-stimulatory, surface-expressed protein. For example, the following antigens are co-stimulatory, surface-expressed proteins: B7-H3 (exemplary antibodies include enoblituzumab and omburtamab), B7-H4, B7-H6, and B7-H7.

In some embodiments, the tumor-associated antigen is a transcription factor or a DNA-binding protein. For example, the following antigens are transcription factors: ETV6-AML, MYCN, PAX3, PAX5, and WT1. The following protein is a DNA-binding protein: BORIS.

In some embodiments, the tumor-associated antigen is an integral membrane protein. For example, the following antigens are integral membrane proteins: SLITRK6 (exemplary antibodies include sirtratumab), UPK2, and UPK3B.

In some embodiments, the tumor-associated antigen is an integrin. For example, the following antigens are integrin antigens: alpha v beta 6, ITGAV (exemplary antibodies include abituzumab), ITGB6, and ITGB8.

In some embodiments, the tumor-associated antigen is a glycolipid. For example, the following are glycolipid antigens: FucGM1, GD2 (exemplary antibodies include dinutuximab), GD3 (exemplary antibodies include mitumomab), GloboH, GM2, and GM3 (exemplary antibodies include racotumomab).

In some embodiments, the tumor-associated antigen is a cell-surface hormone receptor. For example, the following antigens are cell-surface hormone receptors: AMHR2 and androgen receptor.

In some embodiments, the tumor-associated antigen is a transmembrane or membrane-associated protease. For example, the following antigens are transmembrane or membrane-associated proteases: ADAM12, ADAMS, TMPRSS11D, and metalloproteinase.

In some embodiments, the tumor-associated antigen is aberrantly expressed in individuals with cancer. For example, the following antigens may be aberrantly expressed in individuals with cancer: AFP, AGR2, AKAP-4, ARTN, BCR-ABL, C5 complement, CCNB1, CSPG4, CYP1B1, De2-7 EGFR, EGF, Fas-related antigen 1, FBP, G250, GAGE, HAS3, HPV E6 E7, hTERT, IDOL LCK, Legumain, LYPD1, MAD-CT-1, MAD-CT-2, MAGEA3, MAGEA4, MAGEC2, MerTk, ML-IAP, NA17, NY-BR-1, p53, p53 mutant, PAP, PLAVI, polysialic acid, PR1, PSA, Sarcoma translocation breakpoints, SART3, sLe, SSX2, Survivin, Tn, TRAIL, TRAIL1, TRP-2, and XAGE1.

In some embodiments, the antigen is an immune-cell-associated antigen. In some embodiments, the immune-cell-associated antigen is a transmembrane protein. For example, the following antigens are transmembrane proteins: BAFF-R, CD163, CD19, CD20 (exemplary antibodies include rituximab, ocrelizumab, divozilimab; ibritumomab tiuxetan), CD25 (exemplary antibodies include basiliximab), CD274 also known as PD-L1 (exemplary antibodies include adebrelimab, atezolizumab, garivulimab, durvalumab, and avelumab), CD30 (exemplary antibodies include iratumumab and brentuximab), CD33 (exemplary antibodies include lintuzumab), CD352, CD45 (exemplary antibodies include apamistamab), CD47 (exemplary antibodies include letaplimab and magrolimab), CTLA4 (exemplary antibodies include ipilimumab), FASL, IFNAR1 (exemplary antibodies include faralimomab), IFNAR2, LAYN, LILRB2, LILRB4, PD-1 (exemplary antibodies include ipilimumab, nivolumab, pembrolizumab, balstilimab, budigalimab, geptanolimab, toripalimab, and pidilizumabsf), SITZ, and TLR2/4/1 (exemplary antibodies include tomaralimab).

In some embodiments, the immune-cell-associated antigen is a transmembrane transport protein. For example, Mincle is a transmembrane transport protein.

In some embodiments, the immune-cell-associated antigen is a transmembrane or membrane-associated glycoprotein. For example, the following antigens are transmembrane or membrane-associated glycoproteins: CD112, CD155, CD24, CD247, CD28, CD30L, CD37 (exemplary antibodies include lilotomab), CD38 (exemplary antibodies include felzartamab), CD3D, CD3E (exemplary antibodies include foralumab and teplizumab), CD3G, CD44, CLEC12A (exemplary antibodies include tepoditamab), DCIR, DCSIGN, Dectin 1, Dectin 2, ICAM1, LAMP1, Siglecs 1-16, SIRPa, SIRPg, and ULBP1/2/3/4/5/6.

In some embodiments, the immune-cell-associated antigen is a transmembrane or membrane-associated receptor kinase. For example, the following antigens are transmembrane or membrane-associated receptor kinases: Axl (exemplary antibodies include tilvestamab) and FLT3.

In some embodiments, the immune-cell-associated antigen is a membrane-associated or membrane-localized protein. For example, the following antigens are membrane-associated or membrane-localized proteins: CD83, IL1RAP (exemplary antibodies include nidanilimab), OX40, SLAMF7 (exemplary antibodies include elotuzumab), and VSIR.

In some embodiments, the immune-cell-associated antigen is a transmembrane G-protein coupled receptor (GPCR). For example, the following antigens are GPCRs: CCR4 (exemplary antibodies include mogamulizumab-kpkc), CCR8, and CD97.

In some embodiments, the immune-cell-associated antigen is cell-surface-associated or a cell-surface receptor. For example, the following antigens are cell-surface-associated and/or cell-surface receptors: B7-DC, BCMA, CD137, CD2 (exemplary antibodies include siplizumab), CD 244, CD27 (exemplary antibodies include varlilumab), CD278 (exemplary antibodies include feladilimab and vopratelimab), CD3 (exemplary antibodies include otelixizumab and visilizumab), CD40 (exemplary antibodies include dacetuzumab and lucatumumab), CD48, CD5 (exemplary antibodies include zolimomab aritox), CD70 (exemplary antibodies include cusatuzumab and vorsetuzumab), CD74 (exemplary antibodies include milatuzumab), CD79A, CD-262 (exemplary antibodies include tigatuzumab), DR4 (exemplary antibodies include mapatumumab), GITR (exemplary antibodies include ragifilimab), HAVCR2, HLA-DR, HLA-E, HLA-F, HLA-G, LAG-3 (exemplary antibodies include encelimab), MICA, MICB, MRC1, PVRIG, Sialyl-Thomsen-Nouveau Antigen, TIGIT (exemplary antibodies include etigilimab), Trem2, and uPAR.

In some embodiments, the immune-cell-associated antigen is a chemokine receptor or cytokine receptor. For example, the following antigens are chemokine receptors or cytokine receptors: CD115 (exemplary antibodies include axatilimab, cabiralizumab, and emactuzumab), CD123, CXCR4 (exemplary antibodies include ulocuplumab), IL-21R, and IL-5R (exemplary antibodies include benralizumab).

In some embodiments, the immune-cell-associated antigen is a co-stimulatory, surface-expressed protein. For example, the following antigens are co-stimulatory, surface-expressed proteins: B7-H 3 (exemplary antibodies include enoblituzumab and omburtamab), B7-H4, B7-H6, and B7-H7.

In some embodiments, the immune-cell-associated antigen is a peripheral membrane protein. For example, the following antigens are peripheral membrane proteins: B7-1 (exemplary antibodies include galiximab) and B7-2.

In some embodiments, the immune-cell-associated antigen is aberrantly expressed in individuals with cancer. For example, the following antigens may be aberrantly expressed in individuals with cancer: C5 complement, IDO1, LCK, MerTk, and Tyrol.

In some embodiments, the antigen is a stromal-cell-associated antigen. In some embodiments, the stromal-cell-associated antigens is a transmembrane or membrane-associated protein. For example, the following antigens are transmembrane or membrane-associated proteins: FAP (exemplary antibodies include sibrotuzumab), IFNAR1 (exemplary antibodies include faralimomab), and IFNAR2.

In some embodiments, the antigen is CD30. In some embodiments, the antibody is an antibody or antigen-binding fragment that binds to CD30, such as described in International Patent Publication No. WO 02/43661. In some embodiments, the anti-CD30 antibody is cAC10, which is described in International Patent Publication No. WO 02/43661. cAC10 is also known as brentuximab. In some embodiments, the anti-CD30 antibody comprises the CDRs of cAC10. In some embodiments, the CDRs are as defined by the Kabat numbering scheme. In some embodiments, the CDRs are as defined by the Chothia numbering scheme. In some embodiments, the CDRs are as defined by the IMGT numbering scheme. In some embodiments, the CDRs are as defined by the AbM numbering scheme. In some embodiments, the anti-CD30 antibody comprises CDR-H1, CDR-H2, CDR-H3, CDR-L1, CDR-L2, and CDR-L3 comprising the amino acid sequences of SEQ ID NOs: 1, 2, 3, 4, 5, and 6, respectively. In some embodiments, the anti-CD30 antibody comprises a heavy chain variable region comprising an amino acid sequence that is at least 95%, at least 96%, at least 97%, at last 98%, at least 99%, or 100% identical to the amino acid sequence of SEQ ID NO: 7 and a light chain variable region comprising an amino acid sequence that is at least 95% at least 96%, at least 97%, at last 98%, at least 99%, or 100% identical to the amino acid sequence of SEQ ID NO: 8. In some embodiments, the anti-CD30 antibody comprises a heavy chain comprising the amino acid sequence of SEQ ID NO: 9 or SEQ ID NO: 10 and a light chain comprising the amino acid sequence of SEQ ID NO: 11.

In some embodiments, the antigen is CD70. In some embodiments, the antibody is an antibody or antigen-binding fragment that binds to CD70, such as described in International Patent Publication No. WO 2006/113909. In some embodiments, the antibody is a h1F6 anti-CD70 antibody, which is described in International Patent Publication No. WO 2006/113909. h1F6 is also known as vorsetuzumab. In some embodiments, the anti-CD70 antibody comprises a heavy chain variable region comprising the three CDRs of SEQ ID NO:12 and a light chain variable region comprising the three CDRs of SEQ ID NO:13. In some embodiments, the CDRs are as defined by the Kabat numbering scheme. In some embodiments, the CDRs are as defined by the Chothia numbering scheme. In some embodiments, the CDRs are as defined by the IMGT numbering scheme. In some embodiments, the CDRs are as defined by the AbM numbering scheme. In some embodiments, the anti-CD70 antibody comprises a heavy chain variable region comprising an amino acid sequence that is at least 95%, at least 96%, at least 97%, at last 98%, at least 99%, or 100% identical to the amino acid sequence of SEQ ID NO: 12 and a light chain variable region comprising an amino acid sequence that is at least 95% at least 96%, at least 97%, at last 98%, at least 99%, or 100% identical to the amino acid sequence of SEQ ID NO: 13. In some embodiments, the anti-CD30 antibody comprises a heavy chain comprising the amino acid sequence of SEQ ID NO: 14 and a light chain comprising the amino acid sequence of SEQ ID NO: 15.

In some embodiments, the antigen is interleukin-1 receptor accessory protein (IL1RAP). IL1RAP is a co-receptor of the IL1 receptor (IL1R1) and is required for interleukin-1 (IL1) signaling. IL1 has been implicated in the resistance to certain chemotherapy regimens. IL1RAP is overexpressed in various solid tumors, both on cancer cells and in the tumor microenvironment, but has low expression on normal cells. IL1RAP is also overexpressed in hematopoietic stem and progenitor cells, making it a candidate to target for chronic myeloid leukemia (CIVIL). IL1RAP has also been shown to be overexpressed in acute myeloid leukemia (AML). Antibody binding to IL1RAP could block signal transduction from IL-1 and IL-33 into cells and allow NK-cells to recognize tumor cells and subsequent killing by antibody dependent cellular cytotoxicity (ADCC).

In some embodiments, the antigen is ASCT2. ASCT2 is also known as SLC1A5. ASCT2 is a ubiquitously expressed, broad-specificity, sodium-dependent neutral amino acid exchanger. ASCT2 is involved in glutamine transport. ASCT2 is overexpressed in different cancers and is closely related to poor prognosis. Downregulating ASCT2 has been shown to suppress intracellular glutamine levels and downstream glutamine metabolism, including glutathione production. Due to its high expression in many cancers, ASCT2 is a potential therapeutic target. These effects attenuated growth and proliferation, increased apoptosis and autophagy, and increased oxidative stress and mTORC1 pathway suppression in head and neck squamous cell carcinoma (HNSCC). Additionally, silencing ASCT2 improved the response to cetuximab in HNSCC.

In some embodiments, an antibody provided herein binds to TROP2. In some embodiments, the antibody comprises CDR-H1, CDR-H2, CDR-H3, CDR-L1, CDR-L2, and CDR-L3 comprising the amino acid sequences of SEQ ID NOs: 16, 17, 18, 19, 20, and 21, respectively. In some embodiments, the antibody comprises a heavy chain variable region comprising the amino acid sequence of SEQ ID NO: 22 and a light chain variable region comprising the amino acid sequence of SEQ ID NO: 23. In some embodiments, the antibody is sacituzumab. In some embodiments, the antibody comprises CDR-H1, CDR-H2, CDR-H3, CDR-L1, CDR-L2, and CDR-L3 comprising the amino acid sequences of SEQ ID NOs: 24, 25, 26, 27, 28, and 29, respectively. In some embodiments, the antibody comprises a heavy chain variable region comprising the amino acid sequence of SEQ ID NO: 30 and a light chain variable region comprising the amino acid sequence of SEQ ID NO: 31. In some embodiments, the antibody is datopotamab.

In some embodiments, an antibody provided herein binds to MICA. In some embodiments, the antibody comprises CDR-H1, CDR-H2, CDR-H3, CDR-L1, CDR-L2, and CDR-L3 comprising the amino acid sequences of SEQ ID NOs: 32, 33, 34, 35, 36, and 37, respectively. In some embodiments, the antibody comprises a heavy chain variable region comprising the amino acid sequence of SEQ ID NO: 38 and a light chain variable region comprising the amino acid sequence of SEQ ID NO: 39. In some embodiments, the antibody is h1D5v11 hIgG1K. In some embodiments, the antibody comprises CDR-H1, CDR-H2, CDR-H3, CDR-L1, CDR-L2, and CDR-L3 comprising the amino acid sequences of SEQ ID NOs: 40, 41, 42, 43, 44, and 45, respectively. In some embodiments, the antibody comprises a heavy chain variable region comprising the amino acid sequence of SEQ ID NO: 46 and a light chain variable region comprising the amino acid sequence of SEQ ID NO: 47. In some embodiments, the antibody is MICA.36 hIgG1K G236A. In some embodiments, the antibody comprises CDR-H1, CDR-H2, CDR-H3, CDR-L1, CDR-L2, and CDR-L3 comprising the amino acid sequences of SEQ ID NOs: 48, 49, 50, 51, 52, and 53, respectively. In some embodiments, the antibody comprises a heavy chain variable region comprising the amino acid sequence of SEQ ID NO: 54 and a light chain variable region comprising the amino acid sequence of SEQ ID NO: 55. In some embodiments, the antibody is h3F9 H1L3 hIgG1K. In some embodiments, the antibody comprises CDR-H1, CDR-H2, CDR-H3, CDR-L1, CDR-L2, and CDR-L3 comprising the amino acid sequences of SEQ ID NOs: 56, 57, 58, 59, 60, and 61, respectively. In some embodiments, the antibody comprises a heavy chain variable region comprising the amino acid sequence of SEQ ID NO: 62 and a light chain variable region comprising the amino acid sequence of SEQ ID NO: 63. In some embodiments, the antibody is CM33322 Ab28 hIgG1K.

In some embodiments, an antibody provided herein binds to CD24. In some embodiments, the antibody comprises CDR-H1, CDR-H2, CDR-H3, CDR-L1, CDR-L2, and CDR-L3 comprising the amino acid sequences of SEQ ID NOs: 64, 65, 66, 67, 68, and 69, respectively. In some embodiments, the antibody comprises a heavy chain variable region comprising the amino acid sequence of SEQ ID NO: 70 and a light chain variable region comprising the amino acid sequence of SEQ ID NO: 71. In some embodiments, the antibody is SWA11.

In some embodiments, an antibody provided herein binds to ITGay. In some embodiments, the antibody comprises CDR-H1, CDR-H2, CDR-H3, CDR-L1, CDR-L2, and CDR-L3 comprising the amino acid sequences of SEQ ID NOs: 72, 73, 74, 75, 76, and 77, respectively. In some embodiments, the antibody comprises a heavy chain variable region comprising the amino acid sequence of SEQ ID NO: 78 and a light chain variable region comprising the amino acid sequence of SEQ ID NO: 79. In some embodiments, the antibody is intetumumab. In some embodiments, the antibody comprises CDR-H1, CDR-H2, CDR-H3, CDR-L1, CDR-L2, and CDR-L3 comprising the amino acid sequences of SEQ ID NOs: 80, 81, 82, 83, 84, and 85, respectively. In some embodiments, the antibody comprises a heavy chain variable region comprising the amino acid sequence of SEQ ID NO: 86 and a light chain variable region comprising the amino acid sequence of SEQ ID NO: 87. In some embodiments, the antibody is abituzumab.

In some embodiments, an antibody provided herein binds to gpA33. In some embodiments, the antibody comprises CDR-H1, CDR-H2, CDR-H3, CDR-L1, CDR-L2, and CDR-L3 comprising the amino acid sequences of SEQ ID NOs: 88, 89, 90, 91, 92, and 93, respectively. In some embodiments, the antibody comprises a heavy chain variable region comprising the amino acid sequence of SEQ ID NO: 94 and a light chain variable region comprising the amino acid sequence of SEQ ID NO: 95.

In some embodiments, an antibody provided herein binds to IL1Rap. In some embodiments, the antibody comprises CDR-H1, CDR-H2, CDR-H3, CDR-L1, CDR-L2, and CDR-L3 comprising the amino acid sequences of SEQ ID NOs: 96, 97, 98, 99, 100, and 101, respectively. In some embodiments, the antibody comprises a heavy chain variable region comprising the amino acid sequence of SEQ ID NO: 102 and a light chain variable region comprising the amino acid sequence of SEQ ID NO: 103. In some embodiments, the antibody is nidanilimab.

In some embodiments, an antibody provided herein binds to EpCAM. In some embodiments, the antibody comprises CDR-H1, CDR-H2, CDR-H3, CDR-L1, CDR-L2, and CDR-L3 comprising the amino acid sequences of SEQ ID NOs: 104, 105, 106, 017, 108, and 109, respectively. In some embodiments, the antibody comprises a heavy chain variable region comprising the amino acid sequence of SEQ ID NO: 110 and a light chain variable region comprising the amino acid sequence of SEQ ID NO: 111. In some embodiments, the antibody is adecatumumab. In some embodiments, the antibody comprises CDR-H1, CDR-H2, CDR-H3, CDR-L1, CDR-L2, and CDR-L3 comprising the amino acid sequences of SEQ ID NOs: 112, 113, 114, 115, 116, and 117, respectively. In some embodiments, the antibody comprises a heavy chain variable region comprising the amino acid sequence of SEQ ID NO: 118 and a light chain variable region comprising the amino acid sequence of SEQ ID NO: 119. In some embodiments, the antibody is Ep157305. In some embodiments, the antibody comprises CDR-H1, CDR-H2, CDR-H3, CDR-L1, CDR-L2, and CDR-L3 comprising the amino acid sequences of SEQ ID NOs: 120, 121, 122, 123, 124, and 125, respectively. In some embodiments, the antibody comprises a heavy chain variable region comprising the amino acid sequence of SEQ ID NO: 126 and a light chain variable region comprising the amino acid sequence of SEQ ID NO: 127. In some embodiments, the antibody is Ep3-171. In some embodiments, the antibody comprises CDR-H1, CDR-H2, CDR-H3, CDR-L1, CDR-L2, and CDR-L3 comprising the amino acid sequences of SEQ ID NOs: 128, 129, 130, 131, 132, and 133, respectively. In some embodiments, the antibody comprises a heavy chain variable region comprising the amino acid sequence of SEQ ID NO: 134 and a light chain variable region comprising the amino acid sequence of SEQ ID NO: 135. In some embodiments, the antibody is Ep3622w94. In some embodiments, the antibody comprises CDR-H1, CDR-H2, CDR-H3, CDR-L1, CDR-L2, and CDR-L3 comprising the amino acid sequences of SEQ ID NOs: 136, 137, 138, 139, 140, and 141, respectively. In some embodiments, the antibody comprises a heavy chain variable region comprising the amino acid sequence of SEQ ID NO: 142 and a light chain variable region comprising the amino acid sequence of SEQ ID NO: 143. In some embodiments, the antibody is EpING1. In some embodiments, the antibody comprises CDR-H1, CDR-H2, CDR-H3, CDR-L1, CDR-L2, and CDR-L3 comprising the amino acid sequences of SEQ ID NOs: 144, 145, 146, 147, 148, and 149, respectively. In some embodiments, the antibody comprises a heavy chain variable region comprising the amino acid sequence of SEQ ID NO: 150 and a light chain variable region comprising the amino acid sequence of SEQ ID NO: 151. In some embodiments, the antibody is EpAb2-6.

In some embodiments, an antibody provided herein binds to CD352. In some embodiments, the antibody comprises CDR-H1, CDR-H2, CDR-H3, CDR-L1, CDR-L2, and CDR-L3 comprising the amino acid sequences of SEQ ID NOs: 152, 153, 154, 155, 156, and 157, respectively. In some embodiments, the antibody comprises a heavy chain variable region comprising the amino acid sequence of SEQ ID NO: 158 and a light chain variable region comprising the amino acid sequence of SEQ ID NO: 159. In some embodiments, the antibody is h20F3.

In some embodiments, an antibody provided herein binds to CS1. In some embodiments, the antibody comprises CDR-H1, CDR-H2, CDR-H3, CDR-L1, CDR-L2, and CDR-L3 comprising the amino acid sequences of SEQ ID NOs: 160, 161, 162, 163, 164, and 165, respectively. In some embodiments, the antibody comprises a heavy chain variable region comprising the amino acid sequence of SEQ ID NO: 166 and a light chain variable region comprising the amino acid sequence of SEQ ID NO: 167. In some embodiments, the antibody is elotuzumab.

In some embodiments, an antibody provided herein binds to CD38. In some embodiments, the antibody comprises CDR-H1, CDR-H2, CDR-H3, CDR-L1, CDR-L2, and CDR-L3 comprising the amino acid sequences of SEQ ID NOs: 168, 169, 170, 171, 172, and 173, respectively. In some embodiments, the antibody comprises a heavy chain variable region comprising the amino acid sequence of SEQ ID NO: 174 and a light chain variable region comprising the amino acid sequence of SEQ ID NO: 175. In some embodiments, the antibody is daratumumab.

In some embodiments, an antibody provided herein binds to CD25. In some embodiments, the antibody comprises CDR-H1, CDR-H2, CDR-H3, CDR-L1, CDR-L2, and CDR-L3 comprising the amino acid sequences of SEQ ID NOs: 176, 177, 178, 179, 180, and 181, respectively. In some embodiments, the antibody comprises a heavy chain variable region comprising the amino acid sequence of SEQ ID NO: 182 and a light chain variable region comprising the amino acid sequence of SEQ ID NO: 183. In some embodiments, the antibody is daclizumab.

In some embodiments, an antibody provided herein binds to ADAMS. In some embodiments, the antibody comprises CDR-H1, CDR-H2, CDR-H3, CDR-L1, CDR-L2, and CDR-L3 comprising the amino acid sequences of SEQ ID NOs: 184, 185, 186, 187, 188, and 189, respectively. In some embodiments, the antibody comprises a heavy chain variable region comprising the amino acid sequence of SEQ ID NO: 190 and a light chain variable region comprising the amino acid sequence of SEQ ID NO: 191. In some embodiments, the antibody is chMAbA9-A. In some embodiments, the antibody comprises CDR-H1, CDR-H2, CDR-H3, CDR-L1, CDR-L2, and CDR-L3 comprising the amino acid sequences of SEQ ID NOs: 192, 193, 194, 195, 196, and 197, respectively. In some embodiments, the antibody comprises a heavy chain variable region comprising the amino acid sequence of SEQ ID NO: 198 and a light chain variable region comprising the amino acid sequence of SEQ ID NO: 199. In some embodiments, the antibody is hMAbA9-A.

In some embodiments, an antibody provided herein binds to CD59. In some embodiments, the antibody comprises CDR-H1, CDR-H2, CDR-H3, CDR-L1, CDR-L2, and CDR-L3 comprising the amino acid sequences of SEQ ID NOs: 200, 201, 202, 203, 204, and 205, respectively. In some embodiments, the antibody comprises a heavy chain variable region comprising the amino acid sequence of SEQ ID NO: 206 and a light chain variable region comprising the amino acid sequence of SEQ ID NO: 207.

In some embodiments, an antibody provided herein binds to CD25. In some embodiments, the antibody is Clone123.

In some embodiments, an antibody provided herein binds to CD229. In some embodiments, the antibody is h8A10.

In some embodiments, an antibody provided herein binds to CD19. In some embodiments, the antibody comprises CDR-H1, CDR-H2, CDR-H3, CDR-L1, CDR-L2, and CDR-L3 comprising the amino acid sequences of SEQ ID NOs: 208, 209, 210, 211, 212, and 213, respectively. In some embodiments, the antibody comprises a heavy chain variable region comprising the amino acid sequence of SEQ ID NO: 214 and a light chain variable region comprising the amino acid sequence of SEQ ID NO: 215. In some embodiments, the antibody is denintuzumab, which is also known as hBU12. See WO2009052431.

In some embodiments, an antibody provided herein binds to CD70. In some embodiments, the antibody comprises CDR-H1, CDR-H2, CDR-H3, CDR-L1, CDR-L2, and CDR-L3 comprising the amino acid sequences of SEQ ID NOs: 216, 217, 218, 219, 220, and 221, respectively. In some embodiments, the antibody comprises a heavy chain variable region comprising the amino acid sequence of SEQ ID NO: 222 and a light chain variable region comprising the amino acid sequence of SEQ ID NO: 223. In some embodiments, the antibody is vorsetuzumab.

In some embodiments, an antibody provided herein binds to B7H4. In some embodiments, the antibody comprises CDR-H1, CDR-H2, CDR-H3, CDR-L1, CDR-L2, and CDR-L3 comprising the amino acid sequences of SEQ ID NOs: 224, 225, 226, 227, 228, and 229, respectively. In some embodiments, the antibody comprises a heavy chain variable region comprising the amino acid sequence of SEQ ID NO: 230 and a light chain variable region comprising the amino acid sequence of SEQ ID NO: 231. In some embodiments, the antibody is mirzotamab.

In some embodiments, an antibody provided herein binds to CD138. In some embodiments, the antibody comprises CDR-H1, CDR-H2, CDR-H3, CDR-L1, CDR-L2, and CDR-L3 comprising the amino acid sequences of SEQ ID NOs: 232, 233, 234, 235, 236, and 237, respectively. In some embodiments, the antibody comprises a heavy chain variable region comprising the amino acid sequence of SEQ ID NO: 238 and a light chain variable region comprising the amino acid sequence of SEQ ID NO: 239. In some embodiments, the antibody is indatuxumab.

In some embodiments, an antibody provided herein binds to CD166. In some embodiments, the antibody comprises CDR-H1, CDR-H2, CDR-H3, CDR-L1, CDR-L2, and CDR-L3 comprising the amino acid sequences of SEQ ID NOs: 240, 241, 242, 243, 244, and 245, respectively. In some embodiments, the antibody comprises a heavy chain variable region comprising the amino acid sequence of SEQ ID NO: 246 and a light chain variable region comprising the amino acid sequence of SEQ ID NO: 247. In some embodiments, the antibody is praluzatamab.

In some embodiments, an antibody provided herein binds to CD51. In some embodiments, the antibody comprises CDR-H1, CDR-H2, CDR-H3, CDR-L1, CDR-L2, and CDR-L3 comprising the amino acid sequences of SEQ ID NOs: 248, 249, 250, 251, 252, and 253, respectively. In some embodiments, the antibody comprises a heavy chain variable region comprising the amino acid sequence of SEQ ID NO: 254 and a light chain variable region comprising the amino acid sequence of SEQ ID NO: 255. In some embodiments, the antibody is intetumumab.

In some embodiments, an antibody provided herein binds to CD56. In some embodiments, the antibody comprises CDR-H1, CDR-H2, CDR-H3, CDR-L1, CDR-L2, and CDR-L3 comprising the amino acid sequences of SEQ ID NOs: 256, 257, 258, 259, 260, and 261, respectively. In some embodiments, the antibody comprises a heavy chain variable region comprising the amino acid sequence of SEQ ID NO: 262 and a light chain variable region comprising the amino acid sequence of SEQ ID NO: 263. In some embodiments, the antibody is lorvotuzumab.

In some embodiments, an antibody provided herein binds to CD74. In some embodiments, the antibody comprises CDR-H1, CDR-H2, CDR-H3, CDR-L1, CDR-L2, and CDR-L3 comprising the amino acid sequences of SEQ ID NOs: 264, 265, 266, 267, 268, and 269, respectively. In some embodiments, the antibody comprises a heavy chain variable region comprising the amino acid sequence of SEQ ID NO: 270 and a light chain variable region comprising the amino acid sequence of SEQ ID NO: 271. In some embodiments, the antibody is milatuzumab.

In some embodiments, an antibody provided herein binds to CEACAM5. In some embodiments, the antibody comprises CDR-H1, CDR-H2, CDR-H3, CDR-L1, CDR-L2, and CDR-L3 comprising the amino acid sequences of SEQ ID NOs: 272, 273 274, 275, 276, and 277, respectively. In some embodiments, the antibody comprises a heavy chain variable region comprising the amino acid sequence of SEQ ID NO: 278 and a light chain variable region comprising the amino acid sequence of SEQ ID NO: 279. In some embodiments, the antibody is labetuzumab.

In some embodiments, an antibody provided herein binds to CanAg. In some embodiments, the antibody comprises CDR-H1, CDR-H2, CDR-H3, CDR-L1, CDR-L2, and CDR-L3 comprising the amino acid sequences of SEQ ID NOs: 280, 281, 282, 283, 284, and 285, respectively. In some embodiments, the antibody comprises a heavy chain variable region comprising the amino acid sequence of SEQ ID NO: 286 and a light chain variable region comprising the amino acid sequence of SEQ ID NO: 287. In some embodiments, the antibody is cantuzumab.

In some embodiments, an antibody provided herein binds to DLL-3. In some embodiments, the antibody comprises CDR-H1, CDR-H2, CDR-H3, CDR-L1, CDR-L2, and CDR-L3 comprising the amino acid sequences of SEQ ID NOs: 288, 289, 290, 291, 292, and 293, respectively. In some embodiments, the antibody comprises a heavy chain variable region comprising the amino acid sequence of SEQ ID NO: 294 and a light chain variable region comprising the amino acid sequence of SEQ ID NO: 295. In some embodiments, the antibody is rovalpituzumab.

In some embodiments, an antibody provided herein binds to DPEP-3. In some embodiments, the antibody comprises CDR-H1, CDR-H2, CDR-H3, CDR-L1, CDR-L2, and CDR-L3 comprising the amino acid sequences of SEQ ID NOs: 296, 297, 298, 299, 300, and 301, respectively. In some embodiments, the antibody comprises a heavy chain variable region comprising the amino acid sequence of SEQ ID NO: 302 and a light chain variable region comprising the amino acid sequence of SEQ ID NO: 303. In some embodiments, the antibody is tamrintamab.

In some embodiments, an antibody provided herein binds to EGFR. In some embodiments, the antibody comprises CDR-H1, CDR-H2, CDR-H3, CDR-L1, CDR-L2, and CDR-L3 comprising the amino acid sequences of SEQ ID NOs: 304, 305, 306, 307, 308, and 309, respectively. In some embodiments, the antibody comprises a heavy chain variable region comprising the amino acid sequence of SEQ ID NO: 310 and a light chain variable region comprising the amino acid sequence of SEQ ID NO: 311. In some embodiments, the antibody is laprituximab. In some embodiments, the antibody comprises CDR-H1, CDR-H2, CDR-H3, CDR-L1, CDR-L2, and CDR-L3 comprising the amino acid sequences of SEQ ID NOs: 312, 313, 314, 315, 316, and 317, respectively. In some embodiments, the antibody comprises a heavy chain variable region comprising the amino acid sequence of SEQ ID NO: 318 and a light chain variable region comprising the amino acid sequence of SEQ ID NO: 319. In some embodiments, the antibody is losatuxizumab. In some embodiments, the antibody comprises CDR-H1, CDR-H2, CDR-H3, CDR-L1, CDR-L2, and CDR-L3 comprising the amino acid sequences of SEQ ID NOs: 320, 321, 322, 323, 324, and 325, respectively. In some embodiments, the antibody comprises a heavy chain variable region comprising the amino acid sequence of SEQ ID NO: 326 and a light chain variable region comprising the amino acid sequence of SEQ ID NO: 327. In some embodiments, the antibody is serclutamab. In some embodiments, the antibody comprises CDR-H1, CDR-H2, CDR-H3, CDR-L1, CDR-L2, and CDR-L3 comprising the amino acid sequences of SEQ ID NOs: 328, 329, 330, 331, 332, and 333, respectively. In some embodiments, the antibody comprises a heavy chain variable region comprising the amino acid sequence of SEQ ID NO: 334 and a light chain variable region comprising the amino acid sequence of SEQ ID NO: 335. In some embodiments, the antibody is cetuximab.

In some embodiments, an antibody provided herein binds to FRa. In some embodiments, the antibody comprises CDR-H1, CDR-H2, CDR-H3, CDR-L1, CDR-L2, and CDR-L3 comprising the amino acid sequences of SEQ ID NOs: 336, 337, 338, 339, 340, and 341, respectively. In some embodiments, the antibody comprises a heavy chain variable region comprising the amino acid sequence of SEQ ID NO: 342 and a light chain variable region comprising the amino acid sequence of SEQ ID NO: 343. In some embodiments, the antibody is mirvetuximab. In some embodiments, the antibody comprises CDR-H1, CDR-H2, CDR-H3, CDR-L1, CDR-L2, and CDR-L3 comprising the amino acid sequences of SEQ ID NOs: 344, 345, 346, 347, 348, and 349, respectively. In some embodiments, the antibody comprises a heavy chain variable region comprising the amino acid sequence of SEQ ID NO: 350 and a light chain variable region comprising the amino acid sequence of SEQ ID NO: 351. In some embodiments, the antibody is farletuzumab.

In some embodiments, an antibody provided herein binds to MUC-1. In some embodiments, the antibody comprises CDR-H1, CDR-H2, CDR-H3, CDR-L1, CDR-L2, and CDR-L3 comprising the amino acid sequences of SEQ ID NOs: 352, 353, 354, 355, 356, and 357, respectively. In some embodiments, the antibody comprises a heavy chain variable region comprising the amino acid sequence of SEQ ID NO: 358 and a light chain variable region comprising the amino acid sequence of SEQ ID NO: 359. In some embodiments, the antibody is gatipotuzumab.

In some embodiments, an antibody provided herein binds to mesothelin. In some embodiments, the antibody comprises CDR-H1, CDR-H2, CDR-H3, CDR-L1, CDR-L2, and CDR-L3 comprising the amino acid sequences of SEQ ID NOs: 360, 361, 362, 363, 364, and 365, respectively. In some embodiments, the antibody comprises a heavy chain variable region comprising the amino acid sequence of SEQ ID NO: 366 and a light chain variable region comprising the amino acid sequence of SEQ ID NO: 367. In some embodiments, the antibody is anetumab.

In some embodiments, an antibody provided herein binds to ROR-1. In some embodiments, the antibody comprises CDR-H1, CDR-H2, CDR-H3, CDR-L1, CDR-L2, and CDR-L3 comprising the amino acid sequences of SEQ ID NOs: 368, 369, 370, 371, 372, and 373, respectively. In some embodiments, the antibody comprises a heavy chain variable region comprising the amino acid sequence of SEQ ID NO: 374 and a light chain variable region comprising the amino acid sequence of SEQ ID NO: 375. In some embodiments, the antibody is zilovertamab.

In some embodiments, an antibody provided herein binds to ASCT2. In some embodiments, an antibody provided herein binds to B7H4. In some embodiments, the antibody comprises CDR-H1, CDR-H2, CDR-H3, CDR-L1, CDR-L2, and CDR-L3 comprising the amino acid sequences of SEQ ID NOs: 376, 377, 378, 379, 380, and 381, respectively. In some embodiments, the antibody comprises a heavy chain variable region comprising the amino acid sequence of SEQ ID NO: 382 and a light chain variable region comprising the amino acid sequence of SEQ ID NO: 383. In some embodiments, the antibody is 20502. See WO2019040780.

In some embodiments, an antibody provided herein binds to B7-H3. In some embodiments, the antibody comprises CDR-H1, CDR-H2, CDR-H3, CDR-L1, CDR-L2, and CDR-L3 comprising the amino acid sequences of SEQ ID NOs: 384, 385, 386, 387, 388, and 389, respectively. In some embodiments, the antibody comprises a heavy chain variable region comprising the amino acid sequence of SEQ ID NO: 390 and a light chain variable region comprising the amino acid sequence of SEQ ID NO: 391. In some embodiments, the antibody is chAb-A (BRCA84D). In some embodiments, the antibody comprises CDR-H1, CDR-H2, CDR-H3, CDR-L1, CDR-L2, and CDR-L3 comprising the amino acid sequences of SEQ ID NOs: 392, 393, 394, 395, 396, and 397, respectively. In some embodiments, the antibody comprises a heavy chain variable region comprising the amino acid sequence of SEQ ID NO: 398 and a light chain variable region comprising the amino acid sequence of SEQ ID NO: 399. In some embodiments, the antibody is hAb-B. In some embodiments, the antibody comprises CDR-H1, CDR-H2, CDR-H3, CDR-L1, CDR-L2, and CDR-L3 comprising the amino acid sequences of SEQ ID NOs: 400, 401, 402, 403, 404, and 405, respectively. In some embodiments, the antibody comprises a heavy chain variable region comprising the amino acid sequence of SEQ ID NO: 406 and a light chain variable region comprising the amino acid sequence of SEQ ID NO: 407. In some embodiments, the antibody is hAb-C. In some embodiments, the antibody comprises CDR-H1, CDR-H2, CDR-H3, CDR-L1, CDR-L2, and CDR-L3 comprising the amino acid sequences of SEQ ID NOs: 408, 409, 410, 411, 412, and 413, respectively. In some embodiments, the antibody comprises a heavy chain variable region comprising the amino acid sequence of SEQ ID NO: 414 and a light chain variable region comprising the amino acid sequence of SEQ ID NO: 415. In some embodiments, the antibody is hAb-D. In some embodiments, the antibody comprises CDR-H1, CDR-H2, CDR-H3, CDR-L1, CDR-L2, and CDR-L3 comprising the amino acid sequences of SEQ ID NOs: 416, 417, 418, 419, 420, and 421, respectively. In some embodiments, the antibody comprises a heavy chain variable region comprising the amino acid sequence of SEQ ID NO: 422 and a light chain variable region comprising the amino acid sequence of SEQ ID NO: 423. In some embodiments, the antibody is chM30. In some embodiments, the antibody comprises CDR-H1, CDR-H2, CDR-H3, CDR-L1, CDR-L2, and CDR-L3 comprising the amino acid sequences of SEQ ID NOs: 424, 425, 426, 427, 428, and 429, respectively. In some embodiments, the antibody comprises a heavy chain variable region comprising the amino acid sequence of SEQ ID NO: 430 and a light chain variable region comprising the amino acid sequence of SEQ ID NO: 431. In some embodiments, the antibody is hM30-H1-L4. In some embodiments, the antibody comprises CDR-H1, CDR-H2, CDR-H3, CDR-L1, CDR-L2, and CDR-L3 comprising the amino acid sequences of SEQ ID NOs: 432, 433, 434, 435, 436, and 437, respectively. In some embodiments, the antibody comprises a heavy chain variable region comprising the amino acid sequence of SEQ ID NO: 438 and a light chain variable region comprising the amino acid sequence of SEQ ID NO: 439. In some embodiments, the antibody is AbV huAb18-v4. In some embodiments, the antibody comprises CDR-H1, CDR-H2, CDR-H3, CDR-L1, CDR-L2, and CDR-L3 comprising the amino acid sequences of SEQ ID NOs: 440, 441, 442, 443, 444, and 445, respectively. In some embodiments, the antibody comprises a heavy chain variable region comprising the amino acid sequence of SEQ ID NO: 446 and a light chain variable region comprising the amino acid sequence of SEQ ID NO: 447. In some embodiments, the antibody is AbV huAb3-v6. In some embodiments, the antibody comprises CDR-H1, CDR-H2, CDR-H3, CDR-L1, CDR-L2, and CDR-L3 comprising the amino acid sequences of SEQ ID NOs: 448, 449, 450, 451, 452, and 453, respectively. In some embodiments, the antibody comprises a heavy chain variable region comprising the amino acid sequence of SEQ ID NO: 454 and a light chain variable region comprising the amino acid sequence of SEQ ID NO: 455. In some embodiments, the antibody is AbV huAb3-v2.6. In some embodiments, the antibody comprises CDR-H1, CDR-H2, CDR-H3, CDR-L1, CDR-L2, and CDR-L3 comprising the amino acid sequences of SEQ ID NOs: 456, 457, 458, 459, 460, and 461, respectively. In some embodiments, the antibody comprises a heavy chain variable region comprising the amino acid sequence of SEQ ID NO: 462 and a light chain variable region comprising the amino acid sequence of SEQ ID NO: 463. In some embodiments, the antibody is AbV_huAb13-v1-CR. In some embodiments, the antibody comprises CDR-H1, CDR-H2, CDR-H3, CDR-L1, CDR-L2, and CDR-L3 comprising the amino acid sequences of SEQ ID NOs: 464, 465, 466, 467, 468, and 469, respectively. In some embodiments, the antibody comprises a heavy chain variable region comprising the amino acid sequence of SEQ ID NO: 470 and a light chain variable region comprising the amino acid sequence of SEQ ID NO: 471. In some embodiments, the antibody is 8H9-6m. In some embodiments, the antibody comprises a heavy chain variable region comprising the amino acid sequence of SEQ ID NO: 472 and a light chain variable region comprising the amino acid sequence of SEQ ID NO: 473. In some embodiments, the antibody is m8517. In some embodiments, the antibody comprises CDR-H1, CDR-H2, CDR-H3, CDR-L1, CDR-L2, and CDR-L3 comprising the amino acid sequences of SEQ ID NOs: 474, 475, 476, 477, 478, and 479, respectively. In some embodiments, the antibody comprises a heavy chain variable region comprising the amino acid sequence of SEQ ID NO: 480 and a light chain variable region comprising the amino acid sequence of SEQ ID NO: 481. In some embodiments, the antibody is TPP-5706. In some embodiments, the antibody comprises a heavy chain variable region comprising the amino acid sequence of SEQ ID NO: 482 and a light chain variable region comprising the amino acid sequence of SEQ ID NO: 483. In some embodiments, the antibody is TPP-6642. In some embodiments, the antibody comprises a heavy chain variable region comprising the amino acid sequence of SEQ ID NO: 484 and a light chain variable region comprising the amino acid sequence of SEQ ID NO: 485. In some embodiments, the antibody is TPP-6850.

In some embodiments, an antibody provided herein binds to CDCP1. In some embodiments, the antibody is 10D7.

In some embodiments, an antibody provided herein binds to HER3. In some embodiments, the antibody comprises a heavy chain comprising the amino acid sequence of SEQ ID NO: 486 and a light chain comprising the amino acid sequence of SEQ ID NO: 487. In some embodiments, the antibody is patritumab. In some embodiments, the antibody comprises a heavy chain comprising the amino acid sequence of SEQ ID NO: 488 and a light chain comprising the amino acid sequence of SEQ ID NO: 489. In some embodiments, the antibody is seribantumab. In some embodiments, the antibody comprises a heavy chain comprising the amino acid sequence of SEQ ID NO: 490 and a light chain comprising the amino acid sequence of SEQ ID NO: 491. In some embodiments, the antibody is elgemtumab. In some embodiments, the antibody comprises a heavy chain the amino acid sequence of SEQ ID NO: 492 and a light chain comprising the amino acid sequence of SEQ ID NO: 493. In some embodiments, the antibody is lumretuzumab.

In some embodiments, an antibody provided herein binds to RON. In some embodiments, the antibody is Zt/g4.

In some embodiments, an antibody provided herein binds to claudin-2.

In some embodiments, an antibody provided herein binds to HLA-G.

In some embodiments, an antibody provided herein binds to PTK7. In some embodiments, the antibody comprises CDR-H1, CDR-H2, CDR-H3, CDR-L1, CDR-L2, and CDR-L3 comprising the amino acid sequences of SEQ ID NOs: 494, 495, 496, 497, 498, and 499, respectively. In some embodiments, the antibody comprises a heavy chain variable region comprising the amino acid sequence of SEQ ID NO: 500 and a light chain variable region comprising the amino acid sequence of SEQ ID NO: 501. In some embodiments, the antibody is PTK7 mab 1. In some embodiments, the antibody comprises CDR-H1, CDR-H2, CDR-H3, CDR-L1, CDR-L2, and CDR-L3 comprising the amino acid sequences of SEQ ID NOs: 502, 503, 504, 505, 506, and 507, respectively. In some embodiments, the antibody comprises a heavy chain variable region comprising the amino acid sequence of SEQ ID NO: 508 and a light chain variable region comprising the amino acid sequence of SEQ ID NO: 509. In some embodiments, the antibody is PTK7 mab 2. In some embodiments, the antibody comprises CDR-H1, CDR-H2, CDR-H3, CDR-L1, CDR-L2, and CDR-L3 comprising the amino acid sequences of SEQ ID NOs: 510, 511, 512, 513, 514, and 515, respectively. In some embodiments, the antibody comprises a heavy chain variable region comprising the amino acid sequence of SEQ ID NO: 516 and a light chain variable region comprising the amino acid sequence of SEQ ID NO: 517. In some embodiments, the antibody is PTK7 mab 3.

In some embodiments, an antibody provided herein binds to LIV1. In some embodiments, the antibody comprises CDR-H1, CDR-H2, CDR-H3, CDR-L1, CDR-L2, and CDR-L3 comprising the amino acid sequences of SEQ ID NOs: 518, 519, 520, 521, 522, and 523, respectively. In some embodiments, the antibody comprises a heavy chain variable region comprising the amino acid sequence of SEQ ID NO: 524 and a light chain variable region comprising the amino acid sequence of SEQ ID NO: 525. In some embodiments, the antibody is ladiratuzumab, which is also known as hLIV22 and hglg. See WO2012078668.

In some embodiments, an antibody provided herein binds to avb6. In some embodiments, the antibody comprises CDR-H1, CDR-H2, CDR-H3, CDR-L1, CDR-L2, and CDR-L3 comprising the amino acid sequences of SEQ ID NOs: 526, 527, 528, 529, 530, and 531, respectively. In some embodiments, the antibody comprises a heavy chain variable region comprising the amino acid sequence of SEQ ID NO: 532 and a light chain variable region comprising the amino acid sequence of SEQ ID NO: 533. In some embodiments, the antibody is h2A2. In some embodiments, the antibody comprises CDR-H1, CDR-H2, CDR-H3, CDR-L1, CDR-L2, and CDR-L3 comprising the amino acid sequences of SEQ ID NOs: 534, 535, 536, 537, 538, and 539, respectively. In some embodiments, the antibody comprises a heavy chain variable region comprising the amino acid sequence of SEQ ID NO: 540 and a light chain variable region comprising the amino acid sequence of SEQ ID NO: 541. In some embodiments, the antibody is h 15H3.

In some embodiments, an antibody provided herein binds to CD48. In some embodiments, the antibody comprises CDR-H1, CDR-H2, CDR-H3, CDR-L1, CDR-L2, and CDR-L3 comprising the amino acid sequences of SEQ ID NOs: 542, 543, 544, 545, 546, and 547, respectively. In some embodiments, the antibody comprises a heavy chain variable region comprising the amino acid sequence of SEQ ID NO: 548 and a light chain variable region comprising the amino acid sequence of SEQ ID NO: 549. In some embodiments, the antibody is hMEM102. See WO2016149535.

In some embodiments, an antibody provided herein binds to PD-L1. In some embodiments, the antibody comprises CDR-H1, CDR-H2, CDR-H3, CDR-L1, CDR-L2, and CDR-L3 comprising the amino acid sequences of SEQ ID NOs: 550, 551, 552, 553, 554, and 555, respectively. In some embodiments, the antibody comprises a heavy chain variable region comprising the amino acid sequence of SEQ ID NO: 556 and a light chain variable region comprising the amino acid sequence of SEQ ID NO: 557. In some embodiments, the antibody is SG-559-01 LALA mAb.

In some embodiments, an antibody provided herein binds to IGF-1R. In some embodiments, the antibody comprises CDR-H1, CDR-H2, CDR-H3, CDR-L1, CDR-L2, and CDR-L3 comprising the amino acid sequences of SEQ ID NOs: 558, 559, 560, 561, 562, and 563, respectively. In some embodiments, the antibody comprises a heavy chain variable region comprising the amino acid sequence of SEQ ID NO: 564 and a light chain variable region comprising the amino acid sequence of SEQ ID NO: 565. In some embodiments, the antibody is cixutumumab.

In some embodiments, an antibody provided herein binds to claudin-18.2. In some embodiments, the antibody comprises CDR-H1, CDR-H2, CDR-H3, CDR-L1, CDR-L2, and CDR-L3 comprising the amino acid sequences of SEQ ID NOs: 566, 567, 568, 569, 570, and 571, respectively. In some embodiments, the antibody comprises a heavy chain variable region comprising the amino acid sequence of SEQ ID NO: 572 and a light chain variable region comprising the amino acid sequence of SEQ ID NO: 573. In some embodiments, the antibody is zolbetuximab (175D10). In some embodiments, the antibody comprises CDR-H1, CDR-H2, CDR-H3, CDR-L1, CDR-L2, and CDR-L3 comprising the amino acid sequences of SEQ ID NOs: 574, 575, 576, 577, 578, and 579, respectively. In some embodiments, the antibody comprises a heavy chain variable region comprising the amino acid sequence of SEQ ID NO: 580 and a light chain variable region comprising the amino acid sequence of SEQ ID NO: 581. In some embodiments, the antibody is 163E12.

In some embodiments, an antibody provided herein binds to Nectin-4. In some embodiments, the antibody comprises CDR-H1, CDR-H2, CDR-H3, CDR-L1, CDR-L2, and CDR-L3 comprising the amino acid sequences of SEQ ID NOs: 582, 583, 584, 585, 586, and 587, respectively. In some embodiments, the antibody comprises a heavy chain variable region comprising the amino acid sequence of SEQ ID NO: 588 and a light chain variable region comprising the amino acid sequence of SEQ ID NO: 589. In some embodiments, the antibody is enfortumab. See WO 2012047724.

In some embodiments, an antibody provided herein binds to SLTRK6. In some embodiments, the antibody comprises CDR-H1, CDR-H2, CDR-H3, CDR-L1, CDR-L2, and CDR-L3 comprising the amino acid sequences of SEQ ID NOs: 590, 591, 592, 593, 594, and 595, respectively. In some embodiments, the antibody comprises a heavy chain variable region comprising the amino acid sequence of SEQ ID NO: 596 and a light chain variable region comprising the amino acid sequence of SEQ ID NO: 597. In some embodiments, the antibody is sirtratumab.

In some embodiments, an antibody provided herein binds to CD228. In some embodiments, the antibody comprises CDR-H1, CDR-H2, CDR-H3, CDR-L1, CDR-L2, and CDR-L3 comprising the amino acid sequences of SEQ ID NOs: 598, 599, 600, 601, 602, and 603, respectively. In some embodiments, the antibody comprises a heavy chain variable region comprising the amino acid sequence of SEQ ID NO: 604 and a light chain variable region comprising the amino acid sequence of SEQ ID NO: 605. In some embodiments, the antibody is hL49. See WO 2020/163225.

In some embodiments, an antibody provided herein binds to CD142 (tissue factor; TF). In some embodiments, the antibody comprises CDR-H1, CDR-H2, CDR-H3, CDR-L1, CDR-L2, and CDR-L3 comprising the amino acid sequences of SEQ ID NOs: 606, 607, 608, 609, 610, and 611, respectively. In some embodiments, the antibody comprises a heavy chain variable region comprising the amino acid sequence of SEQ ID NO: 612 and a light chain variable region comprising the amino acid sequence of SEQ ID NO: 613. In some embodiments, the antibody is tisotumab. See WO 2010/066803.

In some embodiments, an antibody provided herein binds to STn. In some embodiments, the antibody comprises CDR-H1, CDR-H2, CDR-H3, CDR-L1, CDR-L2, and CDR-L3 comprising the amino acid sequences of SEQ ID NOs: 614, 615, 616, 617, 618, and 619, respectively. In some embodiments, the antibody comprises a heavy chain variable region comprising the amino acid sequence of SEQ ID NO: 620 and a light chain variable region comprising the amino acid sequence of SEQ ID NO: 621. In some embodiments, the antibody is h2G12.

In some embodiments, an antibody provided herein binds to CD20. In some embodiments, the antibody comprises CDR-H1, CDR-H2, CDR-H3, CDR-L1, CDR-L2, and CDR-L3 comprising the amino acid sequences of SEQ ID NOs: 622, 623, 624, 625, 626, and 627, respectively. In some embodiments, the antibody comprises a heavy chain variable region comprising the amino acid sequence of SEQ ID NO: 628 and a light chain variable region comprising the amino acid sequence of SEQ ID NO: 629. In some embodiments, the antibody is rituximab. In some embodiments, the antibody is obinituzumab.

In some embodiments, an antibody provided herein binds to HER2. In some embodiments, the antibody comprises CDR-H1, CDR-H2, CDR-H3, CDR-L1, CDR-L2, and CDR-L3 comprising the amino acid sequences of SEQ ID NOs: 630, 631, 632, 633, 634, and 635, respectively. In some embodiments, the antibody comprises a heavy chain variable region comprising the amino acid sequence of SEQ ID NO: 636 and a light chain variable region comprising the amino acid sequence of SEQ ID NO: 637. In some embodiments, the antibody is trastuzumab.

In some embodiments, an antibody provided herein binds to FLT3.

In some embodiments, an antibody provided herein binds to CD46.

In some embodiments, an antibody provided herein binds to GloboH.

In some embodiments, an antibody provided herein binds to AG7.

In some embodiments, an antibody provided herein binds to mesothelin.

In some embodiments, an antibody provided herein binds to FCRH5.

In some embodiments, an antibody provided herein binds to ETBR.

In some embodiments, an antibody provided herein binds to Tim-1.

In some embodiments, an antibody provided herein binds to SLC44A4.

In some embodiments, an antibody provided herein binds to ENPP3.

In some embodiments, an antibody provided herein binds to CD37.

In some embodiments, an antibody provided herein binds to CA9.

In some embodiments, an antibody provided herein binds to Notch3.

In some embodiments, an antibody provided herein binds to EphA2.

In some embodiments, an antibody provided herein binds to TRFC.

In some embodiments, an antibody provided herein binds to PSMA.

In some embodiments, an antibody provided herein binds to LRRC15.

In some embodiments, an antibody provided herein binds to 5T4.

In some embodiments, an antibody provided herein binds to CD79b. In some embodiments, the antibody comprises CDR-H1, CDR-H2, CDR-H3, CDR-L1, CDR-L2, and CDR-L3 comprising the amino acid sequences of SEQ ID NOs: 638, 639, 640, 641, 642, and 643, respectively. In some embodiments, the antibody comprises a heavy chain variable region comprising the amino acid sequence of SEQ ID NO: 644 and a light chain variable region comprising the amino acid sequence of SEQ ID NO: 645. In some embodiments, the antibody is polatuzumab.

In some embodiments, an antibody provided herein binds to NaPi2B. In some embodiments, the antibody comprises CDR-H1, CDR-H2, CDR-H3, CDR-L1, CDR-L2, and CDR-L3 comprising the amino acid sequences of SEQ ID NOs: 646, 647, 648, 649, 650, and 651, respectively. In some embodiments, the antibody comprises a heavy chain variable region comprising the amino acid sequence of SEQ ID NO: 652 and a light chain variable region comprising the amino acid sequence of SEQ ID NO: 653. In some embodiments, the antibody is lifastuzumab.

In some embodiments, an antibody provided herein binds to Muc16. In some embodiments, the antibody comprises CDR-H1, CDR-H2, CDR-H3, CDR-L1, CDR-L2, and CDR-L3 comprising the amino acid sequences of SEQ ID NOs: 654, 655, 656, 657, 658, and 659, respectively. In some embodiments, the antibody comprises a heavy chain variable region comprising the amino acid sequence of SEQ ID NO: 660 and a light chain variable region comprising the amino acid sequence of SEQ ID NO: 661. In some embodiments, the antibody is sofituzumab.

In some embodiments, an antibody provided herein binds to STEAP1. In some embodiments, the antibody comprises CDR-H1, CDR-H2, CDR-H3, CDR-L1, CDR-L2, and CDR-L3 comprising the amino acid sequences of SEQ ID NOs: 662, 663, 664, 665, 666, and 667, respectively. In some embodiments, the antibody comprises a heavy chain variable region comprising the amino acid sequence of SEQ ID NO: 668 and a light chain variable region comprising the amino acid sequence of SEQ ID NO: 669. In some embodiments, the antibody is vandortuzumab.

In some embodiments, an antibody provided herein binds to BCMA. In some embodiments, the antibody comprises CDR-H1, CDR-H2, CDR-H3, CDR-L1, CDR-L2, and CDR-L3 comprising the amino acid sequences of SEQ ID NOs: 670, 671, 672, 673, 674, and 675, respectively. In some embodiments, the antibody comprises a heavy chain variable region comprising the amino acid sequence of SEQ ID NO: 676 and a light chain variable region comprising the amino acid sequence of SEQ ID NO: 677. In some embodiments, the antibody is belantamab.

In some embodiments, an antibody provided herein binds to c-Met. In some embodiments, the antibody comprises CDR-H1, CDR-H2, CDR-H3, CDR-L1, CDR-L2, and CDR-L3 comprising the amino acid sequences of SEQ ID NOs: 678, 679, 680, 681, 682, and 683, respectively. In some embodiments, the antibody comprises a heavy chain variable region comprising the amino acid sequence of SEQ ID NO: 684 and a light chain variable region comprising the amino acid sequence of SEQ ID NO: 685. In some embodiments, the antibody is telisotuzumab.

In some embodiments, an antibody provided herein binds to EGFR. In some embodiments, the antibody comprises CDR-H1, CDR-H2, CDR-H3, CDR-L1, CDR-L2, and CDR-L3 comprising the amino acid sequences of SEQ ID NOs: 686, 687, 688, 689, 690, and 691, respectively. In some embodiments, the antibody comprises a heavy chain variable region comprising the amino acid sequence of SEQ ID NO: 692 and a light chain variable region comprising the amino acid sequence of SEQ ID NO: 693. In some embodiments, the antibody is depatuxizumab.

In some embodiments, an antibody provided herein binds to SLAMF7. In some embodiments, the antibody comprises CDR-H1, CDR-H2, CDR-H3, CDR-L1, CDR-L2, and CDR-L3 comprising the amino acid sequences of SEQ ID NOs: 694, 695, 696, 697, 698, and 699, respectively. In some embodiments, the antibody comprises a heavy chain variable region comprising the amino acid sequence of SEQ ID NO: 700 and a light chain variable region comprising the amino acid sequence of SEQ ID NO: 701. In some embodiments, the antibody is azintuxizumab.

In some embodiments, an antibody provided herein binds to SLITRK6. In some embodiments, the antibody comprises CDR-H1, CDR-H2, CDR-H3, CDR-L1, CDR-L2, and CDR-L3 comprising the amino acid sequences of SEQ ID NOs: 702, 703, 704, 705, 706, and 707, respectively. In some embodiments, the antibody comprises a heavy chain variable region comprising the amino acid sequence of SEQ ID NO: 708 and a light chain variable region comprising the amino acid sequence of SEQ ID NO: 709. In some embodiments, the antibody is sirtratumab.

In some embodiments, an antibody provided herein binds to C4.4a. In some embodiments, the antibody comprises CDR-H1, CDR-H2, CDR-H3, CDR-L1, CDR-L2, and CDR-L3 comprising the amino acid sequences of SEQ ID NOs: 710, 711, 712, 713, 714, and 715, respectively. In some embodiments, the antibody comprises a heavy chain variable region comprising the amino acid sequence of SEQ ID NO: 716 and a light chain variable region comprising the amino acid sequence of SEQ ID NO: 717. In some embodiments, the antibody is lupartumab.

In some embodiments, an antibody provided herein binds to GCC. In some embodiments, the antibody comprises CDR-H1, CDR-H2, CDR-H3, CDR-L1, CDR-L2, and CDR-L3 comprising the amino acid sequences of SEQ ID NOs: 718, 719, 720, 721, 722, and 723, respectively. In some embodiments, the antibody comprises a heavy chain variable region comprising the amino acid sequence of SEQ ID NO: 724 and a light chain variable region comprising the amino acid sequence of SEQ ID NO: 725. In some embodiments, the antibody is indusatumab.

In some embodiments, an antibody provided herein binds to Axl. In some embodiments, the antibody comprises CDR-H1, CDR-H2, CDR-H3, CDR-L1, CDR-L2, and CDR-L3 comprising the amino acid sequences of SEQ ID NOs: 726, 727, 728, 729, 730, and 731, respectively. In some embodiments, the antibody comprises a heavy chain variable region comprising the amino acid sequence of SEQ ID NO: 732 and a light chain variable region comprising the amino acid sequence of SEQ ID NO: 733. In some embodiments, the antibody is enapotamab.

In some embodiments, an antibody provided herein binds to gpNMB. In some embodiments, the antibody comprises CDR-H1, CDR-H2, CDR-H3, CDR-L1, CDR-L2, and CDR-L3 comprising the amino acid sequences of SEQ ID NOs: 734, 735, 736, 737, 738, and 739, respectively. In some embodiments, the antibody comprises a heavy chain variable region comprising the amino acid sequence of SEQ ID NO: 740 and a light chain variable region comprising the amino acid sequence of SEQ ID NO: 741. In some embodiments, the antibody is glembatumumab.

In some embodiments, an antibody provided herein binds to Prolactin receptor. In some embodiments, the antibody comprises CDR-H1, CDR-H2, CDR-H3, CDR-L1, CDR-L2, and CDR-L3 comprising the amino acid sequences of SEQ ID NOs: 742, 743, 744, 745, 746, and 747, respectively. In some embodiments, the antibody comprises a heavy chain variable region comprising the amino acid sequence of SEQ ID NO: 748 and a light chain variable region comprising the amino acid sequence of SEQ ID NO: 749. In some embodiments, the antibody is rolinsatamab.

In some embodiments, an antibody provided herein binds to FGFR2. In some embodiments, the antibody comprises CDR-H1, CDR-H2, CDR-H3, CDR-L1, CDR-L2, and CDR-L3 comprising the amino acid sequences of SEQ ID NOs: 750, 751, 752, 753, 754, and 755, respectively. In some embodiments, the antibody comprises a heavy chain variable region comprising the amino acid sequence of SEQ ID NO: 756 and a light chain variable region comprising the amino acid sequence of SEQ ID NO: 757. In some embodiments, the antibody is aprutumab.

In some embodiments, an antibody provided herein binds to CDCP1. In some embodiments, the antibody comprises CDR-H1, CDR-H2, CDR-H3, CDR-L1, CDR-L2, and CDR-L3 comprising the amino acid sequences of SEQ ID NOs: 758, 759, 760, 761, 762, and 763, respectively. In some embodiments, the antibody comprises a heavy chain variable region comprising the amino acid sequence of SEQ ID NO: 764 and a light chain variable region comprising the amino acid sequence of SEQ ID NO: 765. In some embodiments, the antibody is Humanized CUB4 #135 HC4-H. In some embodiments, the antibody comprises CDR-H1, CDR-H2, CDR-H3, CDR-L1, CDR-L2, and CDR-L3 comprising the amino acid sequences of SEQ ID NOs: 766, 767, 768, 769, 770, and 771, respectively. In some embodiments, the antibody comprises a heavy chain variable region comprising the amino acid sequence of SEQ ID NO: 772 and a light chain variable region comprising the amino acid sequence of SEQ ID NO: 773. In some embodiments, the antibody is CUB4. In some embodiments, the antibody comprises CDR-H1, CDR-H2, CDR-H3, CDR-L1, CDR-L2, and CDR-L3 comprising the amino acid sequences of SEQ ID NOs: 774, 775, 776, 777, 778, 779, respectively. In some embodiments, the antibody comprises a heavy chain variable region comprising the amino acid sequence of SEQ ID NO: 780 and a light chain variable region comprising the amino acid sequence of SEQ ID NO: 781. In some embodiments, the antibody is CP13E10-WT. In some embodiments, the antibody comprises CDR-H1, CDR-H2, CDR-H3, CDR-L1, CDR-L2, and CDR-L3 comprising the amino acid sequences of SEQ ID NOs: 782, 783, 784, 785, 786, and 787, respectively. In some embodiments, the antibody comprises a heavy chain variable region comprising the amino acid sequence of SEQ ID NO: 788 and a light chain variable region comprising the amino acid sequence of SEQ ID NO: 789. In some embodiments, the antibody is CP13E10-54HCv13-89LCv1.

In some embodiments, an antibody provided herein binds to ASCT2. In some embodiments, the antibody comprises a heavy chain variable region comprising the amino acid sequence of SEQ ID NO: 790 and a light chain variable region comprising the amino acid sequence of SEQ ID NO: 791. In some embodiments, the antibody is KM8094a. In some embodiments, the antibody comprises a heavy chain variable region comprising the amino acid sequence of SEQ ID NO: 792 and a light chain variable region comprising the amino acid sequence of SEQ ID NO: 793. In some embodiments, the antibody is KM8094b. In some embodiments, the antibody comprises CDR-H1, CDR-H2, CDR-H3, CDR-L1, CDR-L2, and CDR-L3 comprising the amino acid sequences of SEQ ID NOs: 794, 795, 796, 797, 798, and 799, respectively. In some embodiments, the antibody comprises a heavy chain variable region comprising the amino acid sequence of SEQ ID NO: 800 and a light chain variable region comprising the amino acid sequence of SEQ ID NO: 801. In some embodiments, the antibody is KM4018.

In some embodiments, an antibody provided herein binds to CD123. In some embodiments, the antibody comprises CDR-H1, CDR-H2, CDR-H3, CDR-L1, CDR-L2, and CDR-L3 comprising the amino acid sequences of SEQ ID NOs: 802, 803, 804, 805, 806, and 807, respectively. In some embodiments, the antibody comprises a heavy chain variable region comprising the amino acid sequence of SEQ ID NO: 808 and a light chain variable region comprising the amino acid sequence of SEQ ID NO: 809. In some embodiments, the antibody is h7G3. See WO 2016201065.

In some embodiments, an antibody provided herein binds to GPC3. In some embodiments, the antibody comprises CDR-H1, CDR-H2, CDR-H3, CDR-L1, CDR-L2, and CDR-L3 comprising the amino acid sequences of SEQ ID NOs: 810, 811, 812, 813, 814, and 815, respectively. In some embodiments, the antibody comprises a heavy chain variable region comprising the amino acid sequence of SEQ ID NO: 816 and a light chain variable region comprising the amino acid sequence of SEQ ID NO: 817. In some embodiments, the antibody is hGPC3-1. See WO 2019161174.

In some embodiments, an antibody provided herein binds to B6A. In some embodiments, the antibody comprises CDR-H1, CDR-H2, CDR-H3, CDR-L1, CDR-L2, and CDR-L3 comprising the amino acid sequences of SEQ ID NOs: 818, 819, 820, 821, 822, and 823, respectively. In some embodiments, the antibody comprises a heavy chain variable region comprising the amino acid sequence of SEQ ID NO: 824 and a light chain variable region comprising the amino acid sequence of SEQ ID NO: 825. In some embodiments, the antibody is h2A2. See PCT/US20/63390. In some embodiments, the antibody comprises CDR-H1, CDR-H2, CDR-H3, CDR-L1, CDR-L2, and CDR-L3 comprising the amino acid sequences of SEQ ID NOs: 826, 827, 828, 829, 830, and 831, respectively. In some embodiments, the antibody comprises a heavy chain variable region comprising the amino acid sequence of SEQ ID NO: 832 and a light chain variable region comprising the amino acid sequence of SEQ ID NO: 833. In some embodiments, the antibody is h15H3. See WO 2013/123152.

In some embodiments, an antibody provided herein binds to PD-L1. In some embodiments, the antibody comprises CDR-H1, CDR-H2, CDR-H3, CDR-L1, CDR-L2, and CDR-L3 comprising the amino acid sequences of SEQ ID NOs: 834, 835, 836, 837, 838, and 839, respectively. In some embodiments, the antibody comprises a heavy chain variable region comprising the amino acid sequence of SEQ ID NO: 840 and a light chain variable region comprising the amino acid sequence of SEQ ID NO: 841. In some embodiments, the antibody is SG-559-01. See PCT/US2020/054037.

In some embodiments, an antibody provided herein binds to TIGIT. In some embodiments, the antibody comprises CDR-H1, CDR-H2, CDR-H3, CDR-L1, CDR-L2, and CDR-L3 comprising the amino acid sequences of SEQ ID NOs: 842, 843, 844, 845, 846, and 847, respectively. In some embodiments, the antibody comprises a heavy chain variable region comprising the amino acid sequence of SEQ ID NO: 848 and a light chain variable region comprising the amino acid sequence of SEQ ID NO: 849. In some embodiments, the antibody is Clone 13 (also known as ADI-23674 or mAb13). See WO 2020041541.

In some embodiments, an antibody provided herein binds to STN. In some embodiments, the antibody comprises CDR-H1, CDR-H2, CDR-H3, CDR-L1, CDR-L2, and CDR-L3 comprising the amino acid sequences of SEQ ID NOs: 850, 851, 852, 853, 854, and 855, respectively. In some embodiments, the antibody comprises a heavy chain variable region comprising the amino acid sequence of SEQ ID NO: 856 and a light chain variable region comprising the amino acid sequence of SEQ ID NO: 857. In some embodiments, the antibody is 2G12-2B2. See WO 2017083582.

In some embodiments, an antibody provided herein binds to CD33. In some embodiments, the antibody comprises CDR-H1, CDR-H2, CDR-H3, CDR-L1, CDR-L2, and CDR-L3 comprising the amino acid sequences of SEQ ID NOs: 858, 859, 860, 861, 862, and 863, respectively. In some embodiments, the antibody comprises a heavy chain variable region comprising the amino acid sequence of SEQ ID NO: 864 and a light chain variable region comprising the amino acid sequence of SEQ ID NO: 865. In some embodiments, the antibody is h2H12. See WO2013173496.

In some embodiments, an antibody provided herein binds to NTBA (also known as CD352). In some embodiments, the antibody comprises CDR-H1, CDR-H2, CDR-H3, CDR-L1, CDR-L2, and CDR-L3 comprising the amino acid sequences of SEQ ID NOs: 866, 867, 868, 869, 870, and 871, respectively. In some embodiments, the antibody comprises a heavy chain variable region comprising the amino acid sequence of SEQ ID NO: 872 and a light chain variable region comprising the amino acid sequence of SEQ ID NO: 873. In some embodiments, the antibody is h20F3 HDLD. See WO 2017004330.

In some embodiments, an antibody provided herein binds to BCMA. In some embodiments, the antibody comprises CDR-H1, CDR-H2, CDR-H3, CDR-L1, CDR-L2, and CDR-L3 comprising the amino acid sequences of SEQ ID NOs: 874, 875, 876, 877, 878, and 879, respectively. In some embodiments, the antibody comprises a heavy chain variable region comprising the amino acid sequence of SEQ ID NO: 880 and a light chain variable region comprising the amino acid sequence of SEQ ID NO: 881. In some embodiments, the antibody is SEA-BCMA (also known as hSG16.17; as used herein, ‘SEA’ denoted antibody afucosylation). See WO 2017/143069.

In some embodiments, an antibody provided herein binds to Tissue Factor (also known as TF). In some embodiments, the antibody comprises CDR-H1, CDR-H2, CDR-H3, CDR-L1, CDR-L2, and CDR-L3 comprising the amino acid sequences of SEQ ID NOs: 882, 883, 884, 885, 886, and 887, respectively. In some embodiments, the antibody comprises a heavy chain variable region comprising the amino acid sequence of SEQ ID NO: 888 and a light chain variable region comprising the amino acid sequence of SEQ ID NO: 889. In some embodiments, the antibody is tisotumab. See WO 2010/066803 and U.S. Pat. No. 9,150,658.

Table of Sequences

| SEQ | | |
|---|---|---|
| ID | | |
| NO | Description | Sequence |
| 1 | cAC10 CDR-H1 | DYYIT |
| 2 | cAC10 CDR-H2 | WIYPGSGNTKYNEKFKG |
| 3 | cAC10 CDR-H3 | YGNYWFAY |
| 4 | cAC10 CDR-L1 | KASQSVDFDGDSYMN |
| 5 | cAC10 CDR-L2 | AASNLES |
| 6 | cAC10 CDR-L3 | QQSNEDPWT |
| 7 | cAC10 VH | QIQLQQSGPEVVKPGASVKISCKASGYTFTDYYITWVKQKPGQGL |
| | | EWIGWIYPGSGNTKY |
| | | NEKFKGKATLTVDTSSSTAFMQLSSLTSEDTAVYFCANYGNYWF |
| | | AYWGQGTQVTVSA |
| 8 | cAC10 VL | DIVLTQSPASLAVSLGQRATISCKASQSVDFDGDSYMNWYQQKPG |
| | | QPPKVLIYAASNLES |
| | | GIPARFSGSGSGTDFTLNIHPVEEEDAATYYCQQSNEDPWTFGGGT |
| | | KLEIK |
| 9 | cAC10 HC | QIQLQQSGPEVVKPGASVKISCKASGYTFTDYYITWVKQKPGQGL |
| | | EWIGWIYPGSGNTKY |
| | | NEKFKGKATLTVDTSSSTAFMQLSSLTSEDTAVYFCANYGNYWF |
| | | AYWGQGTQVTVSAAST |
| | | KGPSVFPLAPSSKSTSGGTAALGCLVKDYFPEPVTVSWNSGALTS |
| | | GVHTFPAVLQSS |
| | | GLYSLSSVVTVPSSSLGTQTYICNVNHKPSNTKVDKKVEPKSCDK |
| | | THTCPPCPAPELLGG |
| | | PSVFLFPPKPKDTLMISRTPEVTCVVVDVSHEDPEVKFNWYVDGV |
| | | EVHNAKTKPREEQYN |
| | | STYRVVSVLTVLHQDWLNGKEYKCKVSNKALPAPIEKTISKAKGQ |
| | | PREPQVYTLPPSRDE |
| | | LTKNQVSLTCLVKGFYPSDIAVEWESNGQPENNYKTTPPVLDSDG |
| | | SFFLYSKLTVDKSRW |
| | | QQGNVFSCSVMHEALHNHYTQKSLSLSPGK |
| 10 | cAC10 HC v2 | QIQLQQSGPEVVKPGASVKISCKASGYTFTDYYITWVKQKPGQGL |
| | | EWIGWIYPGSGNTKY |
| | | NEKFKGKATLTVDTSSSTAFMQLSSLTSEDTAVYFCANYGNYWF |
| | | AYWGQGTQVTVSAAST |
| | | KGPSVFPLAPSSKSTSGGTAALGCLVKDYFPEPVTVSWNSGALTS |
| | | GVHTFPAVLQSS |
| | | GLYSLSSVVTVPSSSLGTQTYICNVNHKPSNTKVDKKVEPKSCDK |
| | | THTCPPCPAPELLGG |
| | | PSVFLFPPKPKDTLMISRTPEVTCVVVDVSHEDPEVKFNWYVDGV |
| | | EVHNAKTKPREEQYN |
| | | STYRVVSVLTVLHQDWLNGKEYKCKVSNKALPAPIEKTISKAKGQ |
| | | PREPQVYTLPPSRDE |
| | | LTKNQVSLTCLVKGFYPSDIAVEWESNGQPENNYKTTPPVLDSDG |
| | | SFFLYSKLTVDKSRW |
| | | QQGNVFSCSVMHEALHNHYTQKSLSLSPG |
| 11 | cAC10 LC | DIVLTQSPASLAVSLGQRATISCKASQSVDFDGDSYMNWYQQKPG |
| | | QPPKVLIYAASNLES |
| | | GIPARFSGSGSGTDFTLNIHPVEEEDAATYYCQQSNEDPWTFGGGT |
| | | KLEIKR |
| | | TVAAPSVFIFPPSDEQLKSGTASVVCLLNNFYPREAKVQWKVDNA |
| | | LQSGNSQESVTEQDS |
| | | KDSTYSLSSTLTLSKADYEKHKVYACEVTHQGLSSPVTKSFNRGE |
| | | C |
| 12 | h1F6 VH | QVQLVQSGAEVKKPGASVKVSCKASGYTFTNYGMNWVRQAPGQ |
| | | GLKWMGWINTYTGEPTY |
| | | ADAFKGRVTMTRDTSISTAYMELSRLRSDDTAVYYCARDYGDYG |
| | | MDYWGQGTTVTVSS |
| 13 | h1F6 VL | DIVMTQSPDSLAVSLGERATINCRASKSVSTSGYSFMHWYQQKPG |
| | | QPPKLLIYLASNLES |
| | | GVPDRFSGSGSGTDFTLTISSLQAEDVAVYYCQHSREVPWTFGQG |
| | | TKVEIK |
| 14 | h1F6 HC | QVQLVQSGAEVKKPGASVKVSCKASGYTFTNYGMNWVRQAPGQ |
| | | GLKWMGWINTYTGEPTY |
| | | ADAFKGRVTMTRDTSISTAYMELSRLRSDDTAVYYCARDYGDYG |
| | | MDYWGQGTTVTVSSAS |
| | | TKGPSVFPLAPSSKSTSGGTAALGCLVKDYFPEPVTVSWNSGALTS |
| | | GVHTFPAVLQSSGL |
| | | YSLSSVVTVPSSSLGTQTYICNVNHKPSNTKVDKKVEPKSCDKTH |
| | | TCPPCPAPELLGGPS |
| | | VFLFPPKPKDTLMISRTPEVTCVVVDVSHEDPEVKFNWYVDGVEV |
| | | HNAKTKPREEQYNST |
| | | YRVVSVLTVLHQDWLNGKEYKCKVSNKALPAPIEKTISKAKGQP |
| | | REPQVYTLPPSRDELT |
| | | KNQVSLTCLVKGFYPSDIAVEWESNGQPENNYKTTPPVLDSDGSF |
| | | FLYSKLTVDKSRWQQ |
| | | GNVFSCSVMHEALHNHYTQKSLSLSPGK |
| 15 | h1F6 LC | DIVMTQSPDSLAVSLGERATINCRASKSVSTSGYSFMHWYQQKPG |
| | | QPPKLLIYLASNLES |
| | | GVPDRFSGSGSGTDFTLTISSLQAEDVAVYYCQHSREVPWTFGQG |
| | | TKVEIKRTVAAPSVF |
| | | IFPPSDEQLKSGTASVVCLLNNFYPREAKVQWKVDNALQSGNSQE |
| | | SVTEQDSKDSTYSLS |
| | | STLTLSKADYEKHKVYACEVTHQGLSSPVTKSFNRGEC |
| 16 | TROP2 CDR-H1 | NYGMN |
| 17 | TROP2 CDR-H2 | WINTYTGEPTYTDDFKG |
| 18 | TROP2 CDR-H3 | GGFGSSYWYFDV |
| 19 | TROP2 CDR-L1 | KASQDVSIAVA |
| 20 | TROP2 CDR-L2 | SASYRYT |
| 21 | TROP2 CDR-L3 | QQHYITPLT |
| 22 | TROP2 VH | QVQLQQSGSELKKPGASVKVSCKASGYTFTNYGMNWVKQAPGQ |
| | | GLKWMGWINTYTGEPT |
| | | YTDDFKGRFAFSLDTSVSTAYLQISSLKADDTAVYFCARGGFGSS |
| | | YWYFDVWGQGSLVTVSS |
| 23 | TROP2 VL | DIQLTQSPSSLSASVGDRVSITCKASQDVSIAVAWYQQKPGKAPKL |
| | | LIYSASYRYTGVP |
| | | DRFSGSGSGTDFTLTISSLQPEDFAVYYCQQHYITPLTFGAGTKVEI |
| | | K |
| 24 | TROP2 CDR-H1 | TAGMQ |
| 25 | TROP2 CDR-H2 | WINTHSGVPKYAEDFKG |
| 26 | TROP2 CDR-H3 | SGFGSSYWYFDV |
| 27 | TROP2 CDR-L1 | KASQDVSTAVA |
| 28 | TROP2 CDR-L2 | SASYRYT |
| 29 | TROP2 CDR-L3 | QQHYITPLT |
| 30 | TROP2 VH | QVQLVQSGAEVKKPGASVKVSCKASGYTFTTAGMQWVRQAPGQ |
| | | GLEWMGWINTHSGVPKYAEDFKGRVTISADTSTSTAYLQLSSLKS |
| | | EDTAVYYCARSGFGSSYWYFDVWGQGTLVTVSS |
| 31 | TROP2 VL | DIQMTQSPSSLSASVGDRVTITCKASQDVSTAVAWYQQKPGKAPK |
| | | LLIYSASYRYTGVPSRFSGSGSGTDFTLTISSLQPEDFAVYYCQQHY |
| | | ITPLTFGQGTKLEIK |
| 32 | MICA CDR-H1 | SQNIY |
| 33 | MICA CDR-H2 | YIEPYNVVPMYNPKFKG |
| 34 | MICA CDR-H3 | SGSSNFDY |
| 35 | MICA CDR-L1 | SASSSISSHYLH |
| 36 | MICA CDR-L2 | RTSNLAS |
| 37 | MICA CDR-L3 | QQGSSLPLT |
| 38 | MICA VH | EIQLVQSGAEVKKPGASVKVSCKASGYAFTSQNIYWVRQAPGQG |
| | | LEWIGYIEPYNVVPMYNPKFKGRATLTVDKSTSTAYLELSSLRSED |
| | | TAVYYCARSGSSNFDYWGQGTLVTVSS |
| 39 | MICA VL | DIQLTQSPSSLSASVGDRVTITCSASSSISSHYLHWYQQKPGKSPKL |
| | | LIYRTSNLASGVPSRFSGSGSGTDYTLTISSLQPEDFATYYCQQGSS |
| | | LPLTFGQGTKVEIK |
| 40 | MICA CDR-H1 | NYAMH |
| 41 | MICA CDR-H2 | LIWYDGSNKFYGDSVKG |
| 42 | MICA CDR-H3 | EGSGHY |
| 43 | MICA CDR-L1 | RASQGISSALA |
| 44 | MICA CDR-L2 | DASSLES |
| 45 | MICA CDR-L3 | QQFNSYPIT |
| 46 | MICA VH | QVQLVESGGGVVQPGRSLRLSCAASGFTFSNYAMHWVRQAPGEG |
| | | LEWVALIWYDGSNKFYGDSVKGRFTISRDNSKNTLYLQMNSLSA |
| | | EDTAVYYCAREGSGHYWGQGTLVTVSS |
| 47 | MICA VL | AIQLTQSPSSLSASVGDRVTITCRASQGISSALAWYQQKPGKVPKS |
| | | LIYDASSLESGVPSRFSGSGSGTDFTLTISSLQPEDFATYYCQQFNS |
| | | YPITFGQGTRLEIK |
| 48 | MICA CDR-H1 | NYAMS |
| 49 | MICA CDR-H2 | YISPGGDYIYYADSVKG |
| 50 | MICA CDR-H3 | DRRHYGSYAMDY |
| 51 | MICA CDR-L1 | RSSKSLLHSNLNTYLY |
| 52 | MICA CDR-L2 | RMSNLAS |
| 53 | MICA CDR-L3 | MQHLEYPFT |
| 54 | MICA VH | QVQLVESGGGLVKPGGSLRLSCAASGFTFSNYAMSWIRQAPGKG |
| | | LEWVSYISPGGDYIYYADSVKGRFTISRDNAKNSLYLQMNSLRAE |
| | | DTAVYYCTTDRRHYGSYAMDYWGQGTLVTVSS |
| 55 | MICA VL | DIVMTQSPLSLPVTPGEPASISCRSSKSLLHSNLNTYLYWFLQKPG |
| | | QSPQILIYRMSNLASGVPDRFSGSGSGTAFTLKISRVEAEDVGVYY |
| | | CMQHLEYPFTFGPGTKLEIK |
| 56 | MICA CDR-H1 | TYAFH |
| 57 | MICA CDR-H2 | GIVPIFGTLKYAQKFQD |
| 58 | MICA CDR-H3 | AIQLEGRPFDH |
| 59 | MICA CDR-L1 | RASQGITSYLA |
| 60 | MICA CDR-L2 | AASALQS |
| 61 | MICA CDR-L3 | QQVNRGAAIT |
| 62 | MICA VH | QVQLVQSGAEVKKPGSSVRVSCRASGGSSTTYAFHWVRQAPGQG |
| | | LEWMGGIVPIFGTLKYAQKFQDRVTLTADKSTGTAYMELNSLRL |
| | | DDTAVYYCARAIQLEGRPFDHWGQGTQVTVSA |
| 63 | MICA VL | DIQLTQSPSFLSASVGDRVTITCRASQGITSYLAWYQQKPGKAPKL |
| | | LIYAASALQSGVPSRFSGRGSGTEFTLTISSLQPEDFATYYCQQVNR |
| | | GAAITFGHGTRLDIK |
| 64 | CD24 CDR-H1 | TYAFH |
| 65 | CD24 CDR-H2 | GIVPIFGTLKYAQKFQD |
| 66 | CD24 CDR-H3 | AIQLEGRPFDH |
| 67 | CD24 CDR-L1 | RASQGITSYLA |
| 68 | CD24 CDR-L2 | AASALQS |
| 69 | CD24 CDR-L3 | QQVNRGAAIT |
| 70 | CD24 VH | QVQLVQSGAEVKKPGSSVRVSCRASGGSSTTYAFHWVRQAPGQG |
| | | LEWMGGIVPIFGTLKYAQKFQDRVTLTADKSTGTAYMELNSLRL |
| | | DDTAVYYCARAIQLEGRPFDHWGQGTQVTVSA |
| 71 | CD24 VL | DIQLTQSPSFLSASVGDRVTITCRASQGITSYLAWYQQKPGKAPKL |
| | | LIYAASALQSGVPS |
| | | RFSGRGSGTEFTLTISSLQPEDFATYYCQQVNRGAAITFGHGTRLDI |
| | | K |
| 72 | ITGav CDR-H1 | RYTMH |
| 73 | ITGav CDR-H2 | VISFDGSNKYYVDSVKG |
| 74 | ITGav CDR-H3 | EARGSYAFDI |
| 75 | ITGav CDR-L1 | RASQSVSSYLA |
| 76 | ITGav CDR-L2 | DASNRAT |
| 77 | ITGav CDR-L3 | QQRSNWPPFT |
| 78 | ITGav VH | QVQLVESGGGVVQPGRSRRLSCAASGFTFSRYTMHWVRQAPGKG |
| | | LEWVAVISFDGSNKYYVDSVKGRFTISRDNSENTLYLQVNILRAE |
| | | DTAVYYCAREARGSYAFDIWGQGTMVTVSS |
| 79 | ITGav VL | EIVLTQSPATLSLSPGERATLSCRASQSVSSYLAWYQQKPGQAPRL |
| | | LIYDASNRATGIPARFSGSGSGTDFTLTISSLEPEDFAVYYCQQRSN |
| | | WPPFTFGPGTKVDIK |
| 80 | ITGav CDR-H1 | SFWMH |
| 81 | ITGav CDR-H2 | YINPRSGYTEYNEIFRD |
| 82 | ITGav CDR-H3 | FLGRGAMDY |
| 83 | ITGav CDR-L1 | RASQDISNYLA |
| 84 | ITGav CDR-L2 | YTSKIHS |
| 85 | ITGav CDR-L3 | QQGNTFPYT |
| 86 | ITGav VH | QVQLQQSGGELAKPGASVKVSCKASGYTFSSFWMHWVRQAPGQ |
| | | GLEWIGYINPRSGYTEYNEIFRDKATMTTDTSTSTAYMELSSLRSE |
| | | DTAVYYCASFLGRGAMDYWGQGTTVTVSS |
| 87 | ITGav VL | DIQMTQSPSSLSASVGDRVTITCRASQDISNYLAWYQQKPGKAPK |
| | | LLIYYTSKIHSGVPSRFSGSGSGTDYTFTISSLQPEDIATYYCQQGN |
| | | TFPYTFGQGTKVEIK |
| 88 | gpA33 CDR-H1 | TSSYYWG |
| 89 | gpA33 CDR-H2 | TIYYNGSTYYSPSLKS |
| 90 | gpA33 CDR-H3 | QGYDIKINIDV |
| 91 | gpA33 CDR-L1 | RASQSVSSYLA |
| 92 | gpA33 CDR-L2 | VASNRAT |
| 93 | gpA33 CDR-L3 | QQRSNWPLT |
| 94 | gpA33 VH | QLQLQESGPGLVKPSETLSLTCTVSGGSISTSSYYWGWIRQPPGKG |
| | | LEWIGTIYYNGSTYYSPSLKSRVSISVDTSKNQFSLKLSSVTAADTS |
| | | VYYCARQGYDIKINIDVWGQGTTVTVSS |
| 95 | gpA33 VL | EIVLTQSPATLSLSPGERATLSCRASQSVSSYLAWYQQKPGQAPRL |
| | | LIYVASNRATGIPARFSGSGSGTDFTLTISSLEPEDFAVYYCQQRSN |
| | | WPLTFGGGTKVEIK |
| 96 | IL1Rap CDR-H1 | SSWMN |
| 97 | IL1Rap CDR-H2 | RIYPGDGNTHYAQKFQG |
| 98 | IL1Rap CDR-H3 | GYLDPMDY |
| 99 | IL1Rap CDR-L1 | QASQGINNYLN |
| 100 | IL1Rap CDR-L2 | YTSGLHA |
| 101 | IL1Rap CDR-L3 | QQYSILPWT |
| 102 | IL1Rap VH | QVQLVQSGAEVKKPGSSVKVSCKASGYAFTSSWMNWVRQAPGQ |
| | | GLEWMGRIYPGDGNTHYAQKFQGRVTLTADKSTSTAYMELSSLR |
| | | SEDTAVYYCGEGYLDPMDYWGQGTLVTVSS |
| 103 | IL1Rap VL | DIQMTQSPSSLSASVGDRVTITCQASQGINNYLNWYQQKPGKAPK |
| | | LLIHYTSGLHAGVPSRFSGSGSGTDYTLTISSLEPEDVATYYCQQY |
| | | SILPWTFGGGTKVEIK |
| 104 | EpCAM CDR-H1 | SYGMH |
| 105 | EpCAM CDR-H2 | VISYDGSNKYYADSVKG |
| 106 | EpCAM CDR-H3 | DMG |
| 107 | EpCAM CDR-L1 | RTSQSISSYLN |
| 108 | EpCAM CDR-L2 | WASTRES |
| 109 | EpCAM CDR-L3 | QQSYDIPYT |
| 110 | EpCAM VH | EVQLLESGGGVVQPGRSLRLSCAASGFTFSSYGMHWVRQAPGKG |
| | | LEWVAVISYDGSNKYYADSVKGRFTISRDNSKNTLYLQMNSLRA |
| | | EDTAVYYCAKDMGWGSGWRPYYYYGMDVWGQGTTVTVSS |
| 111 | EpCAM VL | ELQMTQSPSSLSASVGDRVTITCRTSQSISSYLNWYQQKPGQPPKL |
| | | LIYWASTRESGVPDRFSGSGSGTDFTLTISSLQPEDSATYYCQQSY |
| | | DIPYTFGQGTKLEIK |
| 112 | EpCAM CDR-H1 | NYWMS |
| 113 | EpCAM CDR-H2 | NIKQDGSEKFYADSVKG |
| 114 | EpCAM CDR-H3 | VGPSWEQDY |
| 115 | EpCAM CDR-L1 | TGSSSNIGSYYGVH |
| 116 | EpCAM CDR-L2 | SDTNRPS |
| 117 | EpCAM CDR-L3 | QSYDKGFGHRV |
| 118 | EpCAM VH | EVQLVESGGGLVQPGGSLRLSCAASGFTFSNYWMSWVRQAPGKG |
| | | LEWVANIKQDGSEKFYADSVKGRFTISRDNAKNSLYLQMNSLRA |
| | | EDTAVYYCARVGPSWEQDYWGQGTLVTVSA |
| 119 | EpCAM VL | QSVLTQPPSVSGAPGQRVTISCTGSSSNIGSYYGVHWYQQLPGTAP |
| | | KLLIYSDTNRPSGVPDRFSGSKSGTSASLAITGLQAEDEADYYCQS |
| | | YD |
| 120 | EpCAM CDR-H1 | SYAIS |
| 121 | EpCAM CDR-H2 | GIIPIFGTANYAQKFQG |
| 122 | EpCAM CDR-H3 | GLLWNY |
| 123 | EpCAM CDR-L1 | RASQSVSSNLA |
| 124 | EpCAM CDR-L2 | GASTTAS |
| 125 | EpCAM CDR-L3 | QQYNNWPPAYT |
| 126 | EpCAM VH | QVQLVQSGAEVKKPGSSVKVSCKASGGTFSSYAISWVRQAPGQG |
| | | LEWMGGIIPIFGTANYAQKFQGRVTITADESTSTAYMELSSLRSED |
| | | TAVYYCARGLLWNYWGQGTLVTVSS |
| 127 | EpCAM VL | EIVMTQSPATLSVSPGERATLSCRASQSVSSNLAWYQQKPGQAPR |
| | | LIIYGASTTASGIPARFSASGSGTDFTLTISSLQSEDFAVYYCQQYN |
| | | NWPPAYTFGQGTKLEIK |
| 128 | EpCAM CDR-H1 | NYGMN |
| 129 | EpCAM CDR-H2 | WINTYTGEPTYGEDFKG |
| 130 | EpCAM CDR-H3 | FGNYVDY |
| 131 | EpCAM CDR-L1 | RSSKNLLHSNGITYLY |
| 132 | EpCAM CDR-L2 | QMSNLAS |
| 133 | EpCAM CDR-L3 | AQNLEIPRT |
| 134 | EpCAM VH | QVQLVQSGPEVKKPGASVKVSCKASGYTFTNYGMNWVRQAPGQ |
| | | GLEWMGWINTYTGEPTYGEDFKGRFAFSLDTSASTAYMELSSLRS |
| | | EDTAVYFCARFGNYVDYWGQGSLVTVSS |
| 135 | EpCAM VL | DIVMTQSPLSLPVTPGEPASISCRSSKNLLHSNGITYLYWYLQKPG |
| | | QSPQLLIYQMSNLASGVPDRFSSSGSGTDFTLKISRVEAEDVGVYY |
| | | CAQNLEIPRTFGQGTKVEIK |
| 136 | EpCAM CDR-H1 | KYGMN |
| 137 | EpCAM CDR-H2 | WINTYTEEPTYGDDFKG |
| 138 | EpCAM CDR-H3 | FGSAVDY |
| 139 | EpCAM CDR-L1 | RSSKSLLHSNGITYLY |
| 140 | EpCAM CDR-L2 | QMSNRAS |
| 141 | EpCAM CDR-L3 | AQNLELPRT |
| 142 | EpCAM VH | QIQLVQSGPEVKKPGESVKISCKASGYTFTKYGMNWVKQAPGQG |
| | | LKWMGWINTYTEEPTYGDDFKGRFTFTLDTSTSTAYLEISSLRSED |
| | | TATYFCARFGSAVDYWGQGTLVTVSS |
| 143 | EpCAM VL | DIVMTQSALSNPVTLGESGSISCRSSKSLLHSNGITYLYWYLQKPG |
| | | QSPQLLIYQMSNRASGVPDRFSSSGSGTDFTLKISRVEAEDVGVYY |
| | | CAQNLELPRTFGQGTKLEMKR |
| 144 | EpCAM CDR-H1 | DYSMH |
| 145 | EpCAM CDR-H2 | WINTETGEPTYADDFKG |
| 146 | EpCAM CDR-H3 | TAVY |
| 147 | EpCAM CDR-L1 | RASQEISVSLS |
| 148 | EpCAM CDR-L2 | ATSTLDS |
| 149 | EpCAM CDR-L3 | LQYASYPWT |
| 150 | EpCAM VH | QVKLQESGPELKKPGETVKISCKASGYTFTDYSMHWVKQAPGKG |
| | | LKWMGWINTETGEPTYADDFKGRFAFSLETSASTAYLQINNLKNE |
| | | DTATYFCARTAVYWGQGTTVTVSS |
| 151 | EpCAM VL | DIQMTQSPSSLSASLGERVSLTCRASQEISVSLSWLQQEPDGTIKRL |
| | | IYATSTLDSGVPKRFSGSRSGSDYSLTISSLESEDFVDYYCLQYASY |
| | | PWTFGGGTKLEIKR |
| 152 | CD352 CDR-H1 | NYGMN |
| 153 | CD352 CDR-H2 | WINTYSGEPRYADDFKG |
| 154 | CD352 CDR-H3 | DYGRWYFDV |
| 155 | CD352 CDR-L1 | RASSSVSHMH |
| 156 | CD352 CDR-L2 | ATSNLAS |
| 157 | CD352 CDR-L3 | QQWSSTPRT |
| 158 | CD352 VH | QIQLVQSGSELKKPGASVKVSCKASGYTFTNYGMNWVRQAPGQD |
| | | LKWMGWINTYSGEPRYADDFKGRFVFSLDKSVNTAYLQISSLKAE |
| | | DTAVYYCARDYGRWYFDVWGQGTTVTVSS |
| 159 | CD352 VL | QIVLSQSPATLSLSPGERATMSCRASSSVSHMHWYQQKPGQAPRP |
| | | WIYATSNLASGVPARFSGSGSGTDYTLTISSLEPEDFAVYYCQQWS |
| | | STPRTFGGGTKVEIKR |
| 160 | CS1 CDR-H1 | RYWMS |
| 161 | CS1 CDR-H2 | EINPDSSTINYAPSLKD |
| 162 | CS1 CDR-H3 | PDGNYWYFDV |
| 163 | CS1 CDR-L1 | KASQDVGIAVA |
| 164 | CS1 CDR-L2 | WASTRHT |
| 165 | CS1 CDR-L3 | QQYSSYPYT |
| 166 | CS1 VH | EVQLVESGGGLVQPGGSLRLSCAASGFDFSRYWMSWVRQAPGKG |
| | | LEWIGEINPDSSTINYAPSLKDKFIISRDNAKNSLYLQMNSLRAEDT |
| | | AVYYCARPDGNYWYFDVWGQGTLVTVSS |
| 167 | CS1 VL | DIQMTQSPSSLSASVGDRVTITCKASQDVGIAVAWYQQKPGKVPK |
| | | LLIYWASTRHTGVPDRFSGSGSGTDFTLTISSLQPEDVATYYCQQY |
| | | SSYPYTFGQGTKVEIKR |
| 168 | CD38 CDR-H1 | SFAMS |
| 169 | CD38 CDR-H2 | AISGSGGGTYYADSVKG |
| 170 | CD38 CDR-H3 | DKILWFGEPVFDY |
| 171 | CD38 CDR-L1 | RASQSVSSYLA |
| 172 | CD38 CDR-L2 | DASNRAT |
| 173 | CD38 CDR-L3 | QQRSNWPPT |
| 174 | CD38 VH | EVQLLESGGGLVQPGGSLRLSCAVSGFTFNSFAMSWVRQAPGKG |
| | | LEWVSAISGSGGGTYYADSVKGRFTISRDNSKNTLYLQMNSLRAE |
| | | DTAVYFCAKDKILWFGEPVFDYWGQGTLVTVSS |
| 175 | CD38 VL | EIVLTQSPATLSLSPGERATLSCRASQSVSSYLAWYQQKPGQAPRL |
| | | LIYDASNRATGIPARFSGSGSGTDFTLTISSLEPEDFAVYYCQQRSN |
| | | WPPTFGQGTKVEIKR |
| 176 | CD25 CDR-H1 | SYRMH |
| 177 | CD25 CDR-H2 | YINPSTGYTEYNQKFKD |
| 178 | CD25 CDR-H3 | GGGVFDY |
| 179 | CD25 CDR-L1 | SASSSISYMH |
| 180 | CD25 CDR-L2 | TTSNLAS |
| 181 | CD25 CDR-L3 | HQRSTYPLT |
| 182 | CD25 VH | QVQLVQSGAEVKKPGSSVKVSCKASGYTFTSYRMHWVRQAPGQ |
| | | GLEWIGYINPSTGYTEYNQKFKDKATITADESTNTAYMELSSLRSE |
| | | DTAVYYCARGGGVFDYWGQGTLVTVSS |
| 183 | CD25 VL | DIQMTQSPSTLSASVGDRVTITCSASSSISYMHWYQQKPGKAPKLL |
| | | IYTTSNLASGVPARFSGSGSGTEFTLTISSLQPDDFATYYCHQRSTY |
| | | PLTFGQGTKVEVK |
| 184 | ADAM9 CDR-H1 | SYWM |
| 185 | ADAM9 CDR-H2 | EIIPINGHTNYNEKFKS |
| 186 | ADAM9 CDR-H3 | GGYYYYGSRDYFDY |
| 187 | ADAM9 CDR-L1 | KASQSVDYDGDSYMN |
| 188 | ADAM9 CDR-L2 | AASDLES |
| 189 | ADAM9 CDR-L3 | QQSHEDPFT |
| 190 | ADAM9 VH | QVQLQQPGAELVKPGASVKLSCKASGYTFTSYWMHWVKQRPGQ |
| | | GLEWIGEIIPINGHTNYNEKFKSKATLTLDKSSSTAYMQLSSLASED |
| | | SAVYYCARGGYYYYGSRDYFDYWGQGTTLTVSS |
| 191 | ADAM9 VL | DIVLTQSPASLAVSLGQRATISCKASQSVDYDGDSYMNWYQQIPG |
| | | QPPKLLIYAASDLESGIPARFSGSGSGTDFTLNIHPVEEEDAATYYC |
| | | QQSHEDPFTFGGGTKLEIK |
| 192 | ADAM9 CDR-H1 | SYWM |
| 193 | ADAM9 CDR-H2 | EIIPIFGHTNYNEKFKS |
| 194 | ADAM9 CDR-H3 | GGYYYYPRQGFLDY |
| 195 | ADAM9 CDR-L1 | KASQSVDYDSGDSYMN |
| 196 | ADAM9 CDR-L2 | AASDLES |
| 197 | ADAM9 CDR-L3 | QQSHEDPFT |
| 198 | ADAM9 VH | EVQLVESGGGLVKPGGSLRLSCAASGFTFSSYWMHWVRQAPGKG |
| | | LEWVGEIIPIFGHTNYNEKFKSRFTISLDNSKNTLYLQMGSLRAED |
| | | TAVYYCARGGYYYYPRQGFLDYWGQGTTVTVSS |
| 199 | ADAM9 VL | DIVMTQSPDSLAVSLGERATISCKASQSVDYSGDSYMNWYQQKP |
| | | GQPPKLLIYAASDLESGIPARFSGSGSGTDFTLTISSLEPEDFATYYC |
| | | QQSHEDPFTFGQGTKLEIK |
| 200 | CD59 CDR-H1 | YGMN |
| 201 | CD59 CDR-H2 | YISSSSSTIYADSVKG |
| 202 | CD59 CDR-H3 | GPGMDV |
| 203 | CD59 CDR-L1 | KSSQSVLYSSNNKNYLA |
| 204 | CD59 CDR-L2 | WASTRES |
| 205 | CD59 CDR-L3 | QQYYSTPQLT |
| 206 | CD59 VH | QVQLQQSGGGVVQPGRSLGLSCAASFTFSSYGMNWVRQAPGKGL |
| | | EWVSYISSSSSTIYADSVKGRFTISRDNSKNTLYLQMNSLRAEDTA |
| | | VYYCARGPGMDVWGQGTTVTVS |
| 207 | CD59 VL | DIVLTQSPDSLAVSLGERATINCKSSQSVLYSSNNKNYLAWYQQK |
| | | PGQPPKLLIYWASTRESGVPDRFSGSGSGTDFTPAISSLQAEDVAV |
| | | YYCQQYYSTPQLTFGGGTKVDIK |
| 208 | CD19 CDR-H1 | TSGMGVG |
| 209 | CD19 CDR-H2 | HIWWDDDKRYNPALKS |
| 210 | CD19 CDR-H3 | MELWSYYFDY |
| 211 | CD19 CDR-L1 | SASSSVSYMH |
| 212 | CD19 CDR-L2 | DTSKLAS |
| 213 | CD19 CDR-L3 | FQGSVYPFT |
| 214 | CD19 VH | QVQLQESGPGLVKPSQTLSLTCTVSGGSISTSGMGVGWIRQHPGK |
| | | GLEWIGHIWWDDDKRYNPALKSRVTISVDTSKNQFSLKLSSVTAA |
| | | DTAVYYCARMELWSYYFDYWGQGTLVTVSS |
| 215 | CD19 VL | EIVLTQSPATLSLSPGERATLSCSASSSVSYMHWYQQKPGQAPRLL |
| | | IYDTSKLASGIPARFSGSGSGTDFTLTISSLEPEDVAVYYCFQGSVY |
| | | PFTFGQGTKLEIKR |
| 216 | CD70 CDR-H1 | NYGMN |
| 217 | CD70 CDR-H2 | WINTYTGEPTYADAFKG |
| 218 | CD70 CDR-H3 | DYGDYGMDY |
| 219 | CD70 CDR-L1 | RASKSVSTSGYSFMH |
| 220 | CD70 CDR-L2 | LASNLES |
| 221 | CD70 CDR-L3 | QHSREVPWT |
| 222 | CD70 VH | QVQLVQSGAEVKKPGASVKVSCKASGYTFTNYGMNWVRQAPGQ |
| | | GLKWMGWINTYTGEPTYADAFKGRVTMTRDTSISTAYMELSRLR |
| | | SDDTAVYYCARDYGDYGMDYWGQGTTVTVSS |
| 223 | CD70 VL | DIVMTQSPDSLAVSLGERATINCRASKSVSTSGYSFMHWYQQKPG |
| | | QPPKLLIYLASNLESGVPDRFSGSGSGTDFTLTISSLQAEDVAVYYC |
| | | QHSREVPWTFGQGTKVEIK |
| 224 | B7H4 CDR-H1 | SGYSWH |
| 225 | B7H4 CDR-H2 | YIHSSGSTNYNPSLKS |
| 226 | B7H4 CDR-H3 | YDDYFEY |
| 227 | B7H4 CDR-L1 | KASQNVGFNVA |
| 228 | B7H4 CDR-L2 | SASYRYS |
| 229 | B7H4 CDR-L3 | QQYNWYPFT |
| 230 | B7H4 VH | EVQLQESGPGLVKPSETLSLTCAVTGYSITSGYSWHWIRQFPGNGL |
| | | EWMGYIHSSGSTNYNPSLKSRISISRDTSKNQFFLKLSSVTAADTA |
| | | VYYCAGYDDYFEYWGQGTTVTVSS |
| 231 | B7H4 VL | DIQMTQSPSSLSASVGDRVTITCKASQNVGFNVAWYQQKPGKSPK |
| | | ALIYSASYRYSGVPSRFSGSGSGTDFTLTISSLQPEDFAEYFCQQYN |
| | | WYPFTFGQGTKLEIK |
| 232 | CD138 CDR-H1 | NYWIE |
| 233 | CD138 CDR-H2 | EILPGTGRTIYNEKFKG |
| 234 | CD138 CDR-H3 | RDYYGNFYYAMDY |
| 235 | CD138 CDR-L1 | SASQGINNYLN |
| 236 | CD138 CDR-L2 | YTSTLQS |
| 237 | CD138 CDR-L3 | QQYSKLPRT |
| 238 | CD138 VH | QVQLQQSGSELMMPGASVKISCKATGYTFSNYWIEWVKQRPGHG |
| | | LEWIGEILPGTGRTIY |
| | | NEKFKGKATFTADISSNTVQMQLSSLTSEDSAVYYCARRDYYGNF |
| | | YYAMDYWGQGTSVTVSS |
| 239 | CD138 VL | DIQMTQSTSSLSASLGDRVTISCSASQGINNYLNWYQQKPDGTVEL |
| | | LIYYTSTLQSGVP |
| | | SRFSGSGSGTDYSLTISNLEPEDIGTYYCQQYSKLPRTFGGGTKLEI |
| | | K |
| 240 | CD166 CDR-H1 | TYGMGVG |
| 241 | CD166 CDR-H2 | NIWWSEDKHYSPSLKS |
| 242 | CD166 CDR-H3 | IDYGNDYAFTY |
| 243 | CD166 CDR-L1 | RSSKSLLHSNGITYLY |
| 244 | CD166 CDR-L2 | QMSNLAS |
| 245 | CD166 CDR-L3 | AQNLELPYT |
| 246 | CD166 VH | QITLKESGPTLVKPTQTLTLTCTFSGFSLSTYGMGVGWIRQPPGKA |
| | | LEWLANIWWSEDKHYSPSLKSRLTITKDTSKNQVVLTITNVDPVD |
| | | TATYYCVQIDYGNDYAFTYWGQGTLVTVSS |
| 247 | CD166 VL | DIVMTQSPLSLPVTPGEPASISCRSSKSLLHSNGITYLYWYLQKPGQ |
| | | SPQLLIYQMSNLASGVPDRFSGSGSGTDFTLKISRVEAEDVGVYYC |
| | | AQNLELPYTFGQGTKLEIK |
| 248 | CD51 CDR-H1 | RYTMH |
| 249 | CD51 CDR-H2 | VISFDGSNKYYVDSVKG |
| 250 | CD51 CDR-H3 | EARGSYAFDI |
| 251 | CD51 CDR-L1 | RASQSVSSYLA |
| 252 | CD51 CDR-L2 | DASNRAT |
| 253 | CD51 CDR-L3 | QQRSNWPPFT |
| 254 | CD51 VH | QVQLVESGGGVVQPGRSRRLSCAASGFTFSRYTMHWVRQAPGKG |
| | | LEWVAVISFDGSNKYYVDSVKGRFTISRDNSENTLYLQVNILRAE |
| | | DTAVYYCAREARGSYAFDIWGQGTMVTVSS |
| 255 | CD51 VL | EIVLTQSPATLSLSPGERATLSCRASQSVSSYLAWYQQKPGQAPRL |
| | | LIYDASNRATGIPARFSGSGSGTDFTLTISSLEPEDFAVYYCQQRSN |
| | | WPPFTFGPGTKVDIK |
| 256 | CD56 CDR-H1 | SFGMH |
| 257 | CD56 CDR-H2 | YISSGSFTIYYADSVKG |
| 258 | CD56 CDR-H3 | MRKGYAMDY |
| 259 | CD56 CDR-L1 | RSSQIIIHSDGNTYLE |
| 260 | CD56 CDR-L2 | KVSNRFS |
| 261 | CD56 CDR-L3 | FQGSHVPHT |
| 262 | CD56 VH | QVQLVESGGGVVQPGRSLRLSCAASGFTFSSFGMHWVRQAPGKG |
| | | LEWVAYISSGSFTIYYADSVKGRFTISRDNSKNTLYLQMNSLRAED |
| | | TAVYYCARMRKGYAMDYWGQGTLVTVSS |
| 263 | CD56 VL | DVVMTQSPLSLPVTLGQPASISCRSSQIIIHSDGNTYLEWFQQRPGQ |
| | | SPRRLIYKVSNRFSGVPDRFSGSGSGTDFTLKISRVEAEDVGVYYC |
| | | FQGSHVPHTFGQGTKVEIK |
| 264 | CD74 CDR-H1 | NYGVN |
| 265 | CD74 CDR-H2 | WINPNTGEPTFDDDFKG |
| 266 | CD74 CDR-H3 | SRGKNEAWFAY |
| 267 | CD74 CDR-L1 | RSSQSLVHRNGNTYLH |
| 268 | CD74 CDR-L2 | TVSNRFS |
| 269 | CD74 CDR-L3 | SQSSHVPPT |
| 270 | CD74 VH | QVQLQQSGSELKKPGASVKVSCKASGYTFTNYGVNWIKQAPGQG |
| | | LQWMGWINPNTGEPTFDDDFKGRFAFSLDTSVSTAYLQISSLKAD |
| | | DTAVYFCSRSRGKNEAWFAYWGQGTLVTVSS |
| 271 | CD74 VL | DIQLTQSPLSLPVTLGQPASISCRSSQSLVHRNGNTYLHWFQQRPG |
| | | QSPRLLIYTVSNRFSGVPDRFSGSGSGTDFTLKISRVEAEDVGVYFC |
| | | SQSSHVPPTFGAGTRLEIK |
| 272 | CEACAM5 CDR-H1 | TYWMS |
| 273 | CEACAM5 CDR-H2 | EIHPDSSTINYAPSLKD |
| 274 | CEACAM5 CDR-H3 | LYFGFPWFAY |
| 275 | CEACAM5 CDR-L1 | KASQDVGTSVA |
| 276 | CEACAM5 CDR-L2 | WTSTRHT |
| 277 | CEACAM5 CDR-L3 | QQYSLYRS |
| 278 | CEACAM5 VH | EVQLVESGGGVVQPGRSLRLSCSASGFDFTTYWMSWVRQAPGKG |
| | | LEWIGEIHPDSSTINYAPSLKDRFTISRDNAKNTLFLQMDSLRPEDT |
| | | GVYFCASLYFGFPWFAYWGQGTPVTVSS |
| 279 | CEACAM5 VL | DIQLTQSPSSLSASVGDRVTITCKASQDVGTSVAWYQQKPGKAPK |
| | | LLIYWTSTRHTGVPSRFSGSGSGTDFTFTISSLQPEDIATYYCQQYS |
| | | LYRSFGQGTKVEIK |
| 280 | CanAg CDR-H1 | YYGMN |
| 281 | CanAg CDR-H2 | WIDTTTGEPTYAQKFQG |
| 282 | CanAg CDR-H3 | RGPYNWYFDV |
| 283 | CanAg CDR-L1 | RSSKSLLHSNGNTYLY |
| 284 | CanAg CDR-L2 | RMSNLVS |
| 285 | CanAg CDR-L3 | LQHLEYPFT |
| 286 | CanAg VH | QVQLVQSGAEVKKPGETVKISCKASDYTFTYYGMNWVKQAPGQ |
| | | GLKWMGWIDTTTGEPTYAQKFQGRIAFSLETSASTAYLQIKSLKSE |
| | | DTATYFCARRGPYNWYFDVWGQGTTVTVSS |
| 287 | CanAg VL | DIVMTQSPLSVPVTPGEPVSISCRSSKSLLHSNGNTYLYWFLQRPG |
| | | QSPQLLIYRMSNLVSGVPDRFSGSGSGTAFTLRISRVEAEDVGVYY |
| | | CLQHLEYPFTFGPGTKLELK |
| 288 | DLL-3 CDR-H1 | NYGMN |
| 289 | DLL-3 CDR-H2 | WINTYTGEPTYADDFKG |
| 290 | DLL-3 CDR-H3 | IGDSSPSDY |
| 291 | DLL-3 CDR-L1 | KASQSVSNDVV |
| 292 | DLL-3 CDR-L2 | YASNRYT |
| 293 | DLL-3 CDR-L3 | QQDYTSPWT |
| 294 | DLL-3 VH | QVQLVQSGAEVKKPGASVKVSCKASGYTFTNYGMNWVRQAPGQ |
| | | GLEWMGWINTYTGEPTY |
| | | ADDFKGRVTMTTDTSTSTAYMELRSLRSDDTAVYYCARIGDSSPS |
| | | DYWGQGTLVTVSS |
| 295 | DLL-3 VL | EIVMTQSPATLSVSPGERATLSCKASQSVSNDVVWYQQKPGQAPR |
| | | LLIYYASNRYTGIPA |
| | | RFSGSGSGTEFTLTISSLQSEDFAVYYCQQDYTSPWTFGQGTKLEI |
| | | K |
| 296 | DPEP-3 CDR-H1 | SYWIE |
| 297 | DPEP-3 CDR-H2 | EILPGSGNTYYNERFKD |
| 298 | DPEP-3 CDR-H3 | RAAAYYSNPEWFAY |
| 299 | DPEP-3 CDR-L1 | TASSSVNSFYLH |
| 300 | DPEP-3 CDR-L2 | STSNLAS |
| 301 | DPEP-3 CDR-L3 | HQYHRSPYT |
| 302 | DPEP-3 VH | QVQLVQSGAEVKKPGSSVKVSCKASGGTFSSYWIEWVRQAPGQG |
| | | LEWMGEILPGSGNTYYNERFKDRVTITADESTSTAYMELSSLRSED |
| | | TAVYYCARRAAAYYSNPEWFAYWGQGTLVTVSS |
| 303 | DPEP-3 VL | EIVLTQSPATLSLSPGERATLSCTASSSVNSFYLHWYQQKPGLAPR |
| | | LLIYSTSNLASGIPDRFSGSGSGTDFTLTISRLEPEDFAVYYCHQYH |
| | | RSPYTFGQGTKLEIK |
| 304 | EGFR CDR-H1 | SYWMQ |
| 305 | EGFR CDR-H2 | TIYPGDGDTTYTQKFQG |
| 306 | EGFR CDR-H3 | YDAPGYAMDY |
| 307 | EGFR CDR-L1 | RASQDINNYLA |
| 308 | EGFR CDR-L2 | YTSTLHP |
| 309 | EGFR CDR-L3 | LQYDNLLYT |
| 310 | EGFR VH | QVQLVQSGAEVAKPGASVKLSCKASGYTFTSYWMQWVKQRPGQ |
| | | GLECIGTIYPGDGDTTYTQKFQGKATLTADKSSSTAYMQLSSLRSE |
| | | DSAVYYCARYDAPGYAMDYWGQGTLVTVSS |
| 311 | EGFR VL | DIQMTQSPSSLSASVGDRVTITCRASQDINNYLAWYQHKPGKGPK |
| | | LLIHYTSTLHPGIPSRFSGSGSGRDYSFSISSLEPEDIATYYCLQYDN |
| | | LLYTFGQGTKLEIK |
| 312 | EGFR CDR-H1 | RDFAWN |
| 313 | EGFR CDR-H2 | YISYNGNTRYQPSLKS |
| 314 | EGFR CDR-H3 | ASRGFPY |
| 315 | EGFR CDR-L1 | HSSQDINSNIG |
| 316 | EGFR CDR-L2 | HGTNLDD |
| 317 | EGFR CDR-L3 | VQYAQFPWT |
| 318 | EGFR VH | EVQLQESGPGLVKPSQTLSLTCTVSGYSISRDFAWNWIRQPPGKGL |
| | | EWMGYISYNGNTRYQPSLKSRITISRDTSKNQFFLKLNSVTAADTA |
| | | TYYCVTASRGFPYWGQGTLVTVSS |
| 319 | EGFR VL | DIQMTQSPSSMSVSVGDRVTITCHSSQDINSNIGWLQQKPGKSFKG |
| | | LIYHGTNLDDGVPSRFSGSGSGTDYTLTISSLQPEDFATYYCVQYA |
| | | QFPWTFGGGTKLEIK |
| 320 | EGFR CDR-H1 | RDFAWN |
| 321 | EGFR CDR-H2 | YISYNGNTRYQPSLKS |
| 322 | EGFR CDR-H3 | ASRGFPY |
| 323 | EGFR CDR-L1 | HSSQDINSNIG |
| 324 | EGFR CDR-L2 | HGTNLDD |
| 325 | EGFR CDR-L3 | VQYAQFPWT |
| 326 | EGFR VH | EVQLQESGPGLVKPSQTLSLTCTVSGYSISRDFAWNWIRQPPGKGL |
| | | EWMGYISYNGNTRYQPSLKSRITISRDTSKNQFFLKLNSVTAADTA |
| | | TYYCVTASRGFPYWGQGTLVTVSS |
| 327 | EGFR VL | DIQMTQSPSSMSVSVGDRVTITCHSSQDINSNIGWLQQKPGKSFKG |
| | | LIYHGTNLDDGVPSRFSGSGSGTDYTLTISSLQPEDFATYYCVQYA |
| | | QFPWTFGGGTKLEIK |
| 328 | EGFR CDR-H1 | NYGVH |
| 329 | EGFR CDR-H2 | VIWSGGNTDYNTPFTS |
| 330 | EGFR CDR-H3 | ALTYYDYEFAY |
| 331 | EGFR CDR-L1 | RASQSIGTNIH |
| 332 | EGFR CDR-L2 | YASESIS |
| 333 | EGFR CDR-L3 | QQNNNWPTT |
| 334 | EGFR VH | QVQLKQSGPGLVQPSQSLSITCTVSGFSLINYGVHWVRQSPGKGL |
| | | EWLGVIWSGGNTDYNTPFTSRLSINKDNSKSQVFFKMNSLQSNDT |
| | | AIYYCARALTYYDYEFAYWGQGTLVTVSA |
| 335 | EGFR VL | DILLTQSPVILSVSPGERVSFSCRASQSIGTNIHWYQQRINGSPRLLI |
| | | KYASESISGIPSRFSGSGSGTDFTLSINSVESEDIADYYCQQNNNWP |
| | | TTFGAGTKLELK |
| 336 | FRa CDR-H1 | GYFMN |
| 337 | FRa CDR-H2 | RIHPYDGDTFYNQKFQG |
| 338 | FRa CDR-H3 | YDGSRAMDY |
| 339 | FRa CDR-L1 | KASQSVSFAGTSLMH |
| 340 | FRa CDR-L2 | RASNLEA |
| 341 | FRa CDR-L3 | QQSREYPYT |
| 342 | FRa VH | QVQLVQSGAEVVKPGASVKISCKASGYTFTGYFMNWVKQSPGQS |
| | | LEWIGRIHPYDGDTFY |
| | | NQKFQGKATLTVDKSSNTAHMELLSLTSEDFAVYYCTRYDGSRA |
| | | MDYWGQGTTVTVSS |
| 343 | FRa VL | DIVLTQSPLSLAVSLGQPAIISCKASQSVSFAGTSLMHWYHQKPGQ |
| | | QPRLLIYRASNLEAGVPDRFSGSGSKTDFTLTISPVEAEDAATYYC |
| | | QQSREYPYTFGGGTKLEIK |
| 344 | FRa CDR-H1 | GYGLS |
| 345 | FRa CDR-H2 | MISSGGSYTYYADSVKG |
| 346 | FRa CDR-H3 | HGDDPAWFAY |
| 347 | FRa CDR-L1 | SVSSSISSNNLH |
| 348 | FRa CDR-L2 | GTSNLAS |
| 349 | FRa CDR-L3 | QQWSSYPYMYT |
| 350 | FRa VH | EVQLVESGGGVVQPGRSLRLSCSASGFTFSGYGLSWVRQAPGKGL |
| | | EWVAMISSGGSYTYY |
| | | ADSVKGRFAISRDNAKNTLFLQMDSLRPEDTGVYFCARHGDDPA |
| | | WFAYWGQGTPVTVSS |
| 351 | FRa VL | DIQLTQSPSSLSASVGDRVTITCSVSSSISSNNLHWYQQKPGKAPKP |
| | | WIYGTSNLASGVPSRFSGSGSGTDYTFTISSLQPEDIATYYCQQWS |
| | | SYPYMYTFGQGTKVEIK |
| 352 | MUC-1 CDR-H1 | NYWMN |
| 353 | MUC-1 CDR-H2 | EIRLKSNNYTTHYAESVKG |
| 354 | MUC-1 CDR-H3 | HYYFDY |
| 355 | MUC-1 CDR-L1 | RSSKSLLHSNGITYFF |
| 356 | MUC-1 CDR-L2 | QMSNLAS |
| 357 | MUC-1 CDR-L3 | AQNLELPPT |
| 358 | MUC-1 VH | EVQLVESGGGLVQPGGSMRLSCVASGFPFSNYWMNWVRQAPGK |
| | | GLEWVGEIRLKSNNYTTHYAESVKGRFTISRDDSKNSLYLQMNSL |
| | | KTEDTAVYYCTRHYYFDYWGQGTLVTVSS |
| 359 | MUC-1 VL | DIVMTQSPLSNPVTPGEPASISCRSSKSLLHSNGITYFFWYLQKPGQ |
| | | SPQLLIYQMSNLASGVPDRFSGSGSGTDFTLRISRVEAEDVGVYYC |
| | | AQNLELPPTFGQGTKVEIK |
| 360 | Mesothelin CDR-H1 | SYWIG |
| 361 | Mesothelin CDR-H2 | IIDPGDSRTRYSPSFQG |
| 362 | Mesothelin CDR-H3 | GQLYGGTYMDG |
| 363 | Mesothelin CDR-L1 | TGTSSDIGGYNSVS |
| 364 | Mesothelin CDR-L2 | GVNNRPS |
| 365 | Mesothelin CDR-L3 | SSYDIESATPV |
| 366 | Mesothelin VH | QVELVQSGAEVKKPGESLKISCKGSGYSFTSYWIGWVRQAPGKGL |
| | | EWMGIIDPGDSRTRYSPSFQGQVTISADKSISTAYLQWSSLKASDT |
| | | AMYYCARGQLYGGTYMDGWGQGTLVTVSS |
| 367 | Mesothelin VL | DIALTQPASVSGSPGQSITISCTGTSSDIGGYNSVSWYQQHPGKAPK |
| | | LMIYGVNNRPSGV |
| | | SNRFSGSKSGNTASLTISGLQAEDEADYYCSSYDIESATPVFGGGT |
| | | KLTVL |
| 368 | ROR-1 CDR-H1 | AYNIH |
| 369 | ROR-1 CDR-H2 | SFDPYDGGSSYNQKFKD |
| 370 | ROR-1 CDR-H3 | GWYYFDY |
| 371 | ROR-1 CDR-L1 | RASKSISKYLA |
| 372 | ROR-1 CDR-L2 | SGSTLQS |
| 373 | ROR-1 CDR-L3 | QQHDESPYT |
| 374 | ROR-1 VH | QVQLQESGPGLVKPSQTLSLTCTVSGYAFTAYNIHWVRQAPGQGL |
| | | EWMGSFDPYDGGSSYNQKFKDRLTISKDTSKNQVVLTMTNMDPV |
| | | DTATYYCARGWYYFDYWGHGTLVTVSS |
| 375 | ROR-1 VL | DIVMTQTPLSLPVTPGEPASISCRASKSISKYLAWYQQKPGQAPRL |
| | | LIYSGSTLQSGIPPRFSGSGYGTDFTLTINNIESEDAAYYFCQQHDE |
| | | SPYTFGEGTKVEIK |
| 376 | B7H4 CDR-H1 | GSIKSGSYYWG |
| 377 | B7H4 CDR-H2 | NIYYSGSTYYNPSLRS |
| 378 | B7H4 CDR-H3 | AREGSYPNQFDP |
| 379 | B7H4 CDR-L1 | RASQSVSSNLA |
| 380 | B7H4 CDR-L2 | GASTRAT |
| 381 | B7H4 CDR-L3 | QQYHSFPFT |
| 382 | B7H4 VH | QLQLQESGPGLVKPSETLSLTCTVSGGSIKSGSYYWGWIRQPPGKG |
| | | LEWIGNIYYSGSTY |
| | | YNPSLRSRVTISVDTSKNQFSLKLSSVTAADTAVYYCAREGSYPN |
| | | QFDPWGQGTLVTVSS |
| 383 | B7H4 VL | EIVMTQSPATLSVSPGERATLSCRASQSVSSNLAWYQQKPGQAPR |
| | | LLIYGASTRATGIPA |
| | | RFSGSGSGTEFTLTISSLQSEDFAVYYCQQYHSFPFTFGGGTKVEIK |
| 384 | B7-H3 CDR-H1 | SFGMH |
| 385 | B7-H3 CDR-H2 | YISSDSSAIYY |
| 386 | B7-H3 CDR-H3 | GRENIYYGSRLD |
| 387 | B7-H3 CDR-L1 | KASQNVD |
| 388 | B7-H3 CDR-L2 | SASYRYSGVPD |
| 389 | B7-H3 CDR-L3 | QQYNNYPFTFGS |
| 390 | B7-H3 VH | DVQLVESGGGLVQPGGSRKLSCAASGFTFSSFGMHWVRQAPEKG |
| | | LEWVAYISSDSSAIYY |
| | | ADTVKGRFTISRDNPKNTLFLQMTSLRSEDTAMYYCGRGRENIYY |
| | | GSRLDYWGQGTTLTVSS |
| 391 | B7-H3 VL | DIAMTQSQKFMSTSVGDRVSVTCKASQNVDTNVAWYQQKPGQS |
| | | PKALIYSASYRYSGVPD |
| | | RFTGSGSGTDFTLTINNVQSEDLAEYFCQQYNNYPFTFGSGTKLEI |
| | | K |
| 392 | B7-H3 CDR-H1 | SYWMQWVRQA |
| 393 | B7-H3 CDR-H2 | TIYPGDGDTRY |
| 394 | B7-H3 CDR-H3 | RGIPRLWYFDVM |
| 395 | B7-H3 CDR-L1 | ITCRASQDIS |
| 396 | B7-H3 CDR-L2 | YTSRLHSGVPS |
| 397 | B7-H3 CDR-L3 | QQGNTLPPFTGG |
| 398 | B7-H3 VH | DVQLVESGGGLVQPGGSRKLSCAASGFTFSSFGMHWVRQAPEKG |
| | | LEWVAYISSDSSAIYY |
| | | ADTVKGRFTISRDNPKNTLFLQMTSLRSEDTAMYYCGRGRENIYY |
| | | GSRLDYWGQGTTLTVSS |
| 399 | B7-H3 VL | DIAMTQSQKFMSTSVGDRVSVTCKASQNVDTNVAWYQQKPGQS |
| | | PKALIYSASYRYSGVPD |
| | | RFTGSGSGTDFTLTINNVQSEDLAEYFCQQYNNYPFTFGSGTKLEI |
| | | K |
| 400 | B7-H3 CDR-H1 | SYGMSWVRQA |
| 401 | B7-H3 CDR-H2 | INSGGSNTYY |
| 402 | B7-H3 CDR-H3 | HDGGAMDYW |
| 403 | B7-H3 CDR-L1 | ITCRASESIYSYLA |
| 404 | B7-H3 CDR-L2 | NTKTLPE |
| 405 | B7-H3 CDR-L3 | HHYGTPPWTFG |
| 406 | B7-H3 VH | EVQLVESGGGLVKPGGSLRLSCAASGFTFSSYGMSWVRQAPGKG |
| | | LEWVATINSGGSNTYY |
| | | PDSLKGRFTISRDNAKNSLYLQMNSLRAEDTAVYYCARHDGGAM |
| | | DYWGQGTTVTVSS |
| 407 | B7-H3 VL | DIQMTQSPSSLSASVGDRVTITCRASESIYSYLAWYQQKPGKAPKL |
| | | LVYNTKTLPEGVPSRFSGSGSGTDFTLTISSLQPEDFATYYCQHHY |
| | | GTPPWTFGQGTRLEIK |
| 408 | B7-H3 CDR-H1 | SFGMHWVRQA |
| 409 | B7-H3 CDR-H2 | ISSGSGTIYYADTVKGRFTI |
| 410 | B7-H3 CDR-H3 | HGYRYEGFDYWG |
| 411 | B7-H3 CDR-L1 | ITCKASQNVDTNVA |
| 412 | B7-H3 CDR-L2 | SASYRYSGVPS |
| 413 | B7-H3 CDR-L3 | QQYNNYPFTFGQ |
| 414 | B7-H3 VH | EVQLVESGGGLVQPGGSLRLSCAASGFTFSSFGMHWVRQAPGKG |
| | | LEWVAYISSGSGTIY |
| | | YADTVKGRFTISRDNAKNSLYLQMNSLRAEDTAVYYCARHGYRY |
| | | EGFDYWGQGTTVTVSS |
| 415 | B7-H3 VL | DIQMTQSPSFLSASVGDRVTITCKASQNVDTNVAWYQQKPGKAP |
| | | KALIYSASYRYSGVPSRFSGSGSGTDFTLTISSLQPEDFAEYFCQQY |
| | | NNYPFTFGQGTKLEIK |
| 416 | B7-H3 CDR-H1 | NYVMH |
| 417 | B7-H3 CDR-H2 | YINPYNDDVKYNEKFKG |
| 418 | B7-H3 CDR-H3 | WGYYGSPLYYFDY |
| 419 | B7-H3 CDR-L1 | RASSRLIYMH |
| 420 | B7-H3 CDR-L2 | ATSNLAS |
| 421 | B7-H3 CDR-L3 | QQWNSNPPT |
| 422 | B7-H3 VH | EVQLQQSGPELVKPGASVKMSCKASGYTFTNYVMHWVKQKPGQ |
| | | GLEWIGYINPYNDDVKYNEKFKGKATQTSDKSSSTAYMELSSLTS |
| | | EDSAVYYCARWGYYGSPLYYFDYWGQGTTLTVSS |
| 423 | B7-H3 VL | QIVLSQSPTILSASPGEKVTMTCRASSRLIYMHWYQQKPGSSPKPW |
| | | IYATSNLASGVPAR |
| | | FSGSGSGTSYSLTISRVEAEDAATYYCQQWNSNPPTFGTGTKLELK |
| 424 | B7-H3 CDR-H1 | NYVMH |
| 425 | B7-H3 CDR-H2 | YINPYNDDVKYNEKFKG |
| 426 | B7-H3 CDR-H3 | WGYYGSPLYYFDY |
| 427 | B7-H3 CDR-L1 | RASSRLIYMH |
| 428 | B7-H3 CDR-L2 | ATSNLAS |
| 429 | B7-H3 CDR-L3 | QQWNSNPPT |
| 430 | B7-H3 VH | QVQLVQSGAEVKKPGSSVKVSCKASGYTFTNYVMHWVRQAPGQ |
| | | GLEWMGYINPYNDDVKYNE |
| | | KFKGRVTITADESTSTAYMELSSLRSEDTAVYYCARWGYYGSPLY |
| | | YFDYWGQGTLVTVSS |
| 431 | B7-H3 VL | EIVLTQSPATLSLSPGERATLSCRASSRLIYMHWYQQKPGQAPRPLI |
| | | YATSNLASGIPARFSGSGSGTDFTLTISSLEPEDFAVYYCQQWNSN |
| | | PPTFGQGTKVEIK |
| 432 | B7-H3 CDR-H1 | GYSFTSYTIH |
| 433 | B7-H3 CDR-H2 | YINPNSRNTDYAQKFQG |
| 434 | B7-H3 CDR-H3 | YSGSTPYWYFDV |
| 435 | B7-H3 CDR-L1 | RASSSVSYMN |
| 436 | B7-H3 CDR-L2 | ATSNLAS |
| 437 | B7-H3 CDR-L3 | QQWSSNPLT |
| 438 | B7-H3 VH | EVQLVQSGAEVKKPGSSVKVSCKASGYSFTSYTIHWVRQAPGQG |
| | | LEWMGYINPNSRNTDYAQKFQGRVTLTADKSTSTAYMELSSLRSE |
| | | DTAVYYCARYSGSTPYWYFDVWGQGTTVTVSS |
| 439 | B7-H3 VL | DIQMTQSPSSLSASVGDRVTITCKASQNVGFNVAWYQQKPGKSPK |
| | | ALIYSASYRYSGVPSRFSGSGSGTDFTLTISSLQPEDFAEYFCQQYN |
| | | WYPFTFGQGTKLEIK |
| 440 | B7-H3 CDR-H1 | GYTFSSYWMH |
| 441 | B7-H3 CDR-H2 | LIHPDSGSTNYNEMFKN |
| 442 | B7-H3 CDR-H3 | GGRLYFD |
| 443 | B7-H3 CDR-L1 | RSSQSLVHSNGDTYLR |
| 444 | B7-H3 CDR-L2 | KVSNRFS |
| 445 | B7-H3 CDR-L3 | SQSTHVPYT |
| 446 | B7-H3 VH | EVQLVQSGAEVKKPGSSVKVSCKASGYTFSSYWMHWVRQAPGQ |
| | | GLEWIGLIHPDSGSTNYNEMFKNRATLTVDRSTSTAYVELSSLRSE |
| | | DTAVYFCAGGGRLYFDYWGQGTTVTVSS |
| 447 | B7-H3 VL | DVVMTQSPLSLPVTPGEPASISCRSSQSLVHSNGDTYLRWYLQKP |
| | | GQSPQLLIYKVSNRFSGVPDRFSGSGSGTDFTLKISRVEAEDVGVY |
| | | YCSQSTHVPYTFGGGTKVEIK |
| 448 | B7-H3 CDR-H1 | GYTFSSYWMH |
| 449 | B7-H3 CDR-H2 | LIHPESGSTNYNEMFKN |
| 450 | B7-H3 CDR-H3 | GGRLYFDY |
| 451 | B7-H3 CDR-L1 | RSSQSLVHSNQDTYLR |
| 452 | B7-H3 CDR-L2 | KVSNRFS |
| 453 | B7-H3 CDR-L3 | SQSTHVPYT |
| 454 | B7-H3 VH | EVQLVQSGAEVKKPGSSVKVSCKASGYTFSSYWMHWVRQAPGQ |
| | | GLEWIGLIHPESGSTNY |
| | | NEMFKNRATLTVDRSTSTAYMELSSLRSEDTAVYYCAGGGRLYF |
| | | DYWGQGTTVTVSS |
| 455 | B7-H3 VL | DIVMTQSPLSLPVTPGEPASISCRSSQSLVHSNQDTYLRWYLQKPG |
| | | QSPQLLIYKVSNRF |
| | | SGVPDRFSGSGSGTDFTLKKISRVEAEDVGVYYCSQSTHVPYTFG |
| | | GGTKVEIK |
| 456 | B7-H3 CDR-H1 | TGYSITSGYSWH |
| 457 | B7-H3 CDR-H2 | YIHSSGSTNYNPSLKS |
| 458 | B7-H3 CDR-H3 | YDDYFEY |
| 459 | B7-H3 CDR-L1 | KASQNVGFNVAW |
| 460 | B7-H3 CDR-L2 | SASYRYS |
| 461 | B7-H3 CDR-L3 | QQYNWYPFT |
| 462 | B7-H3 VH | EVQLQESGPGLVKPSETLSLTCAVTGYSITSGYSWHWIRQFPGNGL |
| | | EWMGYIHSSGSTNY |
| | | NPSLKSRISISRDTSKNQFFLKLSSVTAADTAVYYCAGYDDYFEY |
| | | WGQGTTVTVSS |
| 463 | B7-H3 VL | DIQMTQSPSSLSASVGDRVTITCKASQNVGGFNVAWYQQKPGKSP |
| | | KALIYSASYRYSGV |
| | | PSRFSGSGSGTDFTLTISSLQPEDFAEYFCQQYNWYPFTFGQGTKL |
| | | EIK |
| 464 | B7-H3 CDR-H1 | NYDIN |
| 465 | B7-H3 CDR-H2 | WIGWIFPGDDSTQYNEKFKG |
| 466 | B7-H3 CDR-H3 | QTTGTWFAY |
| 467 | B7-H3 CDR-L1 | RASQSISDYLY |
| 468 | B7-H3 CDR-L2 | YASQSIS |
| 469 | B7-H3 CDR-L3 | CQNGHSFPL |
| 470 | B7-H3 VH | QVQLVQSGAEVVKPGASVKLSCKTSGYTFTNYDINWVRQRPGQG |
| | | LEWIGWIFPGDDSTQY |
| | | NEKFKGKATLTTDTSTSTAYMELSSLRSEDTAVYFCARQTTGTWF |
| | | AYWGQGTLVTVSS |
| 471 | B7-H3 VL | EIVMTQSPATLSVSPGERVTLSCRASQSISDYLYWYQQKSHESPRL |
| | | LIKYASQSISGIPA |
| | | RFSGSGSGSEFTLTINSVEPEDVGVYYCQNGHSFPLTFGQGTKLEL |
| | | K |
| 472 | B7-H3 VH | QVQLQQSGAEVKKPGSSVKVSCKASGGTFSSYAISWVRQAPGQG |
| | | LEWMGGIIPILGIAN |
| | | YAQKFQGRVTITADESTSTAYMELSSLRSEDTAVYYCARGGSGSY |
| | | HMDVWGKGTTVTVSS |
| 473 | B7-H3 VL | EIVLTQSPATLSLSPGERATLSCRASQSVSSYLAWYQQKPGQAPRL |
| | | LIYDASNRATGIP |
| | | ARFSGSGSGTDFTLTISSLEPEDFAVYYCQQRSNWPPRITFGQGTRL |
| | | EIK |
| 474 | B7-H3 CDR-H1 | IYNVH |
| 475 | B7-H3 CDR-H2 | TIFPGNGDTSYNQKFKD |
| 476 | B7-H3 CDR-H3 | WDDGNVGFAH |
| 477 | B7-H3 CDR-L1 | RASENINNYLT |
| 478 | B7-H3 CDR-L2 | HAKTLAE |
| 479 | B7-H3 CDR-L3 | QHHYGTPPT |
| 480 | B7-H3 VH | QVQLQQPGAELVKPGASVKMSCKASGYTFTIYNVHWIKQTPGQG |
| | | LEWMGTIFPGNGDTSY |
| | | NQKFKDKATLTTDKSSKTAYMQLNSLTSEDSAVYYCARWDDGN |
| | | VGFAHWGQGTLVTVSA |
| 481 | B7-H3 VL | DIQMTQSPASLSASVGETVTITCRASENINNYLTWFQQKQGKSPQL |
| | | LVYHAKTLAEGVPS |
| | | RFSGSGSGTQFSLKINSLQPEDFGSYYCQHHYGTPPTFGGGTKLEI |
| | | K |
| 482 | B7-H3 VH | EVQLVQSGAEVKKPGASVKVSCKASGYTFTIYNVHWVRQAPGQG |
| | | LEWMGTIFPGNGDTS |
| | | YNQKFKDKVTMTTDTSTSTAYMELSSLRSEDTAVYYCARWDDG |
| | | NVGFAHWGQGTLVTVSS |
| 483 | B7-H3 VL | DIQMTQSPSSLSASVGDRVTITCRASENINNYLTWFQQKQGKSPQL |
| | | LIYHAKTLAEGVP |
| | | SRFSGSGSGTDFTLTISSLQPEDFATYYCQHHYGTPPTFGGGTKVEI |
| | | K |
| 484 | B7-H3 VH | EVQLVQSGAEVKKPGASVKVSCKASGYTFTIYNVHWIRQAPGQG |
| | | LEWMGTIFPGNGDTSY |
| | | NQKFKDRATLTTDKSTKTAYMELRSLRSDDTAVYYCARWDDGN |
| | | VGFAHWGQGTLVTVSS |
| 485 | B7-H3 VL | DIQMTQSPSSLSASVGDRVTITCRASENINNYLTWFQQKPGKAPKL |
| | | LVYHAKTLAEGVPS |
| | | RFSGSGSGTQFTLTISSLQPEDFATYYCQHHYGTPPTFGQGTKLEIK |
| 486 | HER3 H | QVQLQQWGAGLLKPSETLSLTCAVYGGSFSGYYWSWIRQPPGKG |
| | | LEWIGEINHSGSTNYN |
| | | PSLKSRVTISVETSKNQFSLKLSSVTAADTAVYYCARDKWTWYFD |
| | | LWGRGTLVTVSSAST |
| | | KGPSVFPLAPSSKSTSGGTAALGCLVKDYFPEPVTVSWNSGALTS |
| | | GVHTFPAVLQSSGL |
| | | YSLSSVVTVPSSSLGTQTYICNVNHKPSNTKVDKRVEPKSCDKTHT |
| | | CPPCPAPELLGGPSVFLFPPKPKDTLMISRTPEVTCVVVDVSHEDPE |
| | | VKFNWYVDGVEVHNAKTKPREEQYNSTYRVVSVLTVLHQDWLN |
| | | GKEYKCKVSNKALPAPIEKTISKAKGQPREPQVYTLPPSREEMTKN |
| | | QVSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 487 | HER3 L | DIEMTQSPDSLAVSLGERATINCRSSQSVLYSSSNRNYLAWYQQN |
| | | PGQPPKLLIYWASTRESGVPDRFSGSGSGTDFTLTISSLQAEDVAV |
| | | YYCQQYYSTPRTFGQGTKVEIKRTVAAPSVFIFPPSDEQLKSGTAS |
| | | VVCLLNNFYPREAKVQWKVDNALQSGNSQESVTEQDSKDSTYSL |
| | | SSTLTLSKADYEKHKVYACEVTHQGLSSPVTKSFNRGEC |
| 488 | HER3 H | EVQLLESGGGLVQPGGSLRLSCAASGFTFSHYVMAWVRQAPGKG |
| | | LEWVSSISSSGGWTLY |
| | | ADSVKGRFTISRDNSKNTLYLQMNSLRAEDTAVYYCTRGLKMATI |
| | | FDYWGQGTLVTVSSA |
| | | STKGPSVFPLAPCSRSTSESTAALGCLVKDYFPEPVTVSWNSGALT |
| | | SGVHTFPAVLQSSG |
| | | LYSLSSVVTVPSSNFGTQTYTCNVDHKPSNTKVDKTVERKCCVEC |
| | | PPCPAPPVAGPSVFL |
| | | FPPKPKDTLMISRTPEVTCVVVDVSHEDPEVQFNWYVDGVEVHN |
| | | AKTKPREEQFNSTFRV |
| | | VSVLTVVHQDWLNGKEYKCKVSNKGLPAPIEKTISKTKGQPREPQ |
| | | VYTLPPSREEMTKNQ |
| | | VSLTCLVKGFYPSDIAVEWESNGQPENNYKTTPPMLDSDGSFFLY |
| | | SKLTVDKSRWQQGNV |
| | | FSCSVMHEALHNHYTQKSLSLSPGK |
| 489 | HER3 L | QSALTQPASVSGSPGQSITISCTGTSSDVGSYNVVSWYQQHPGKAP |
| | | KLIIYEVSQRPSGVSNRFSGSKSGNTASLTISGLQTEDEADYYCCSY |
| | | AGSSIFVIFGGGTKVTVLGQPKAAPSVTLFPPSSEELQANKATLVC |
| | | LVSDFYPGAVTVAWKADGSPVKVGVETTKPSKQSNNKYAASSYL |
| | | SLTPEQWKSHRSYSCRVTHEGSTVEKTVAPAECS |
| 490 | HER3 H | EVQLLESGGGLVQPGGSLRLSCAASGFTFSSYAMSWVRQAPGKG |
| | | LEWVSAINSQGKSTYYADSVKGRFTISRDNSKNTLYLQMNSLRAE |
| | | DTAVYYCARWGDEGFDIWGQGTLVTVSSASTKGPSVFPLAPSSKS |
| | | TSGGTAALGCLVKDYFPEPVTVSWNSGALTSGVHTFPAVLQSSGL |
| | | YSLSSVVTVPSSSLGTQTYICNVNHKPSNTKVDKRVEPKSCDKTHT |
| | | CPPCPAPELLGGPSVFLFPPKPKDTLMISRTPEVTCVVVDVSHEDPE |
| | | VKFNWYVDGVEVHNAKTKPREEQYNSTYRVVSVLTVLHQDWLN |
| | | GKEYKCKVSNKALPAPIEKTISKAKGQPREPQVYTLPPSREEMTKN |
| | | QVSLTCLVKGFYPSDIAVEWESNGQPENNYKTTPPVLDSDGSFFL |
| | | YSKLTVDKSRWQQGNVFSCSVMHEALHNHYTQKSLSLSPGK |
| 491 | HER3 L | DIQMTQSPSSLSASVGDRVTITCRASQGISNWLAWYQQKPGKAPK |
| | | LLIYGASSLQSGVPSRFSGSGSGTDFTLTISSLQPEDFATYYCQQYS |
| | | SFPTTFGQGTKVEIKRTVAAPSVFIFPPSDEQLKSGTASVVCLLNNF |
| | | YPREAKVQWKVDNALQSGNSQESVTEQDSKDSTYSLSSTLTLSKA |
| | | DYEKHKVYACEVTHQGLSSPVTKSFNRGEC |
| 492 | HER3 H | QVQLVQSGAEVKKPGASVKVSCKASGYTFRSSYISWVRQAPGQG |
| | | LEWMGWIYAGTGSPSYNQKLQGRVTMTTDTSTSTAYMELRSLRS |
| | | DDTAVYYCARHRDYYSNSLTYWGQGTLVTVSSASTKGPSVFPLA |
| | | PSSKSTSGGTAALGCLVKDYFPEPVTVSWNSGALTSGVHTFPAVL |
| | | QSSGLYSLSSVVTVPSSSLGTQTYICNVNHKPSNTKVDKKVEPKSC |
| | | DKTHTCPPCPAPELLGGPSVFLFPPKPKDTLMISRTPEVTCVVVDV |
| | | SHEDPEVKFNWYVDGVEVHNAKTKPREEQYNSTYRVVSVLTVLH |
| | | QDWLNGKEYKCKVSNKALPAPIEKTISKAKGQPREPQVYTLPPSR |
| | | DELTKNQVSLTCLVKGFYPSDIAVEWESNGQPENNYKTTPPVLDS |
| | | DGSFFLYSKLTVDKSRWQQGNVFSCSVMHEALHNHYTQKSLSLS |
| | | PG |
| 493 | HER3 L | DIVMTQSPDSLAVSLGERATINCKSSQSVLNSGNQKNYLTWYQQK |
| | | PGQPPKLLIYWASTRESGVPDRFSGSGSGTDFTLTISSLQAEDVAV |
| | | YYCQSDYSYPYTFGQGTKLEIKRTVAAPSVFIFPPSDEQLKSGTAS |
| | | VVCLLNNFYPREAKVQWKVDNALQSGNSQESVTEQDSKDSTYSL |
| | | SSTLTLSKADYEKHKVYACEVTHQGLSSPVTKSFNRGEC |
| 494 | PTK7 CDR-H1 | TSNMGVG |
| 495 | PTK7 CDR-H2 | HIWWDDDKYYSPSLKS |
| 496 | PTK7 CDR-H3 | SNYGYAWFAY |
| 497 | PTK7 CDR-L1 | KASQDIYPYLN |
| 498 | PTK7 CDR-L2 | RTNRLLD |
| 499 | PTK7 CDR-L3 | LQYDEFPLT |
| 500 | PTK7 VH | QITLKESGPTLVKPTQTLTLTCTFSGFSLSTSNMGVGWIRQPPGKA |
| | | LEWLAHIWWDDDKYYSPSLKSRLTITKDTSKNQVVLTMTNMDPV |
| | | DTATYYCVRSNYGYAWFAYWGQGTLVTVSS |
| 501 | PTK7 VL | DIQMTQSPSSLSASVGDRVTITCKASQDIYPYLNWFQQKPGKAPKT |
| | | LIYRTNRLLDGVPS |
| | | RFSGSGSGTDFTFTISSLQPEDIATYYCLQYDEFPLTFGAGTKLEIK |
| 502 | PTK7 CDR-H1 | DYAVH |
| 503 | PTK7 CDR-H2 | VISTYNDYTYNNQDFKG |
| 504 | PTK7 CDR-H3 | GNSYFYALDY |
| 505 | PTK7 CDR-L1 | RASESVDSYGKSFMH |
| 506 | PTK7 CDR-L2 | RASNLES |
| 507 | PTK7 CDR-L3 | QQSNEDPWT |
| 508 | PTK7 VH | QVQLVQSGPEVKKPGASVKVSCKASGYTFTDYAVHWVRQAPGK |
| | | RLEWIGVISTYNDYTY |
| | | NNQDFKGRVTMTRDTSASTAYMELSRLRSEDTAVYYCARGNSYF |
| | | YALDYWGQGTSVTVSS |
| 509 | PTK7 VL | EIVLTQSPATLSLSPGERATLSCRASESVDSYGKSFMHWYQQKPG |
| | | QAPRLLIYRASNLES |
| | | GIPARFSGSGSGTDFTLTISSLEPEDFAVYYCQQSNEDPWTFGGGT |
| | | KLEIK |
| 510 | PTK7 CDR-H1 | RYWMS |
| 511 | PTK7 CDR-H2 | DLNPDSSAINYVDSVKG |
| 512 | PTK7 CDR-H3 | ITTLVPYTMDF |
| 513 | PTK7 CDR-L1 | ITNTDIDDDMN |
| 514 | PTK7 CDR-L2 | EGNGLRP |
| 515 | PTK7 CDR-L3 | LQSDNLPLT |
| 516 | PTK7 VH | EVQLVESGGGLVQPGGSLRLSCAASGFDFSRYWMSWVRQAPGKG |
| | | LEWIGDLNPDSSAINY |
| | | VDSVKGRFTISRDNAKNSLYLQMNSLRAEDTAVYYCTLITTLVPY |
| | | TMDFWGQGTSVTVSS |
| 517 | PTK7 VL | ETTLTQSPAFMSATPGDKVNISCITNTDIDDDMNWYQQKPGEAAI |
| | | LLISEGNGLRPGIPPRFSGSGYGTDFTLTINNIESEDAAYYFCLQSD |
| | | NLPLTFGSGTKLEIK |
| 518 | LIV1 CDR-H1 | DYYMH |
| 519 | LIV1 CDR-H2 | WIDPENGDTEYGPKFQG |
| 520 | LIV1 CDR-H3 | HNAHYGTWFAY |
| 521 | LIV1 CDR-L1 | RSSQSLLHSSGNTYLE |
| 522 | LIV1 CDR-L2 | KISTRFS |
| 523 | LIV1 CDR-L3 | FQGSHVPYT |
| 524 | LIV1 VH | QVQLVQSGAEVKKPGASVKVSCKASGLTIEDYYMHWVRQAPGQ |
| | | GLEWMGWIDPENGDTEY |
| | | GPKFQGRVTMTRDTSINTAYMELSRLRSDDTAVYYCAVHNAHYG |
| | | TWFAYWGQGTLVTVSS |
| 525 | LIV1 VL | DVVMTQSPLSLPVTLGQPASISCRSSQSLLHSSGNTYLEWYQQRPG |
| | | QSPRPLIYKISTRFSGVPDRFSGSGSGTDFTLKISRVEAEDVGVYYC |
| | | FQGSHVPYTFGGGTKVEIK |
| 526 | avb6 CDR-H1 | DYNVN |
| 527 | avb6 CDR-H2 | VINPKYGTTRYNQKFKG |
| 528 | avb6 CDR-H3 | GLNAWDY |
| 529 | avb6 CDR-L1 | GASENIYGALN |
| 530 | avb6 CDR-L2 | GATNLED |
| 531 | avb6 CDR-L3 | QNVLTTPYT |
| 532 | avb6 VH | QFQLVQSGAEVKKPGASVKVSCKASGYSFTDYNVNWVRQAPGQ |
| | | GLEWIGVINPKYGTTRY |
| | | NQKFKGRATLTVDKSTSTAYMELSSLRSEDTAVYYCTRGLNAWD |
| | | YWGQGTLVTVSS |
| 533 | avb6 VL | DIQMTQSPSSLSASVGDRVTITCGASENIYGALNWYQQKPGKAPK |
| | | LLIYGATNLEDGVPS |
| | | RFSGSGSGRDYTFTISSLQPEDIATYYCQNVLTTPYTFGQGTKLEIK |
| 534 | avb6 CDR-H1 | GYFMN |
| 535 | avb6 CDR-H2 | LINPYNGDSFYNQKFKG |
| 536 | avb6 CDR-H3 | GLRRDFDY |
| 537 | avb6 CDR-L1 | KSSQSLLDSDGKTYLN |
| 538 | avb6 CDR-L2 | LVSELDS |
| 539 | avb6 CDR-L3 | WQGTHFPRT |
| 540 | avb6 VH | QVQLVQSGAEVKKPGASVKVSCKASGYSFSGYFMNWVRQAPGQ |
| | | GLEWMGLINPYNGDSFY |
| | | NQKFKGRVTMTRQTSTSTVYMELSSLRSEDTAVYYCVRGLRRDF |
| | | DYWGQGTLVTVSS |
| 541 | avb6 VL | DVVMTQSPLSLPVTLGQPASISCKSSQSLLDSDGKTYLNWLFQRPG |
| | | QSPRRLIYLVSELD |
| | | SGVPDRFSGSGSGTDFTLKISRVEAEDVGVYYCWQGTHFPRTFGG |
| | | GTKLEIK |
| 542 | CD48 CDR-H1 | DFGMN |
| 543 | CD48 CDR-H2 | WINTFTGEPSYGNVFKG |
| 544 | CD48 CDR-H3 | RHGNGNVFDS |
| 545 | CD48 CDR-L1 | RASQSIGSNIH |
| 546 | CD48 CDR-L2 | YTSESIS |
| 547 | CD48 CDR-L3 | QQSNSWPLT |
| 548 | CD48 VH | QVQLVQSGSELKKPGASVKVSCKASGYTFTDFGMNWVRQAPGQ |
| | | GLEWMGWINTFTGEPSYGNVFKGRFVFSLDTSVSTAYLQISSLKA |
| | | EDTAVYYCARRHGNGNVFDSWGQGTLVTVSS |
| 549 | CD48 VL | EIVLTQSPDFQSVTPKEKVTITCRASQSIGSNIHWYQQKPDQSPKLL |
| | | IKYTSESISGVPSRFSGSGSGTDFTLTINSLEAEDAATYYCQQSNSW |
| | | PLTFGGGTKVEIKR |
| 550 | PD-L1 CDR-H1 | TAAIS |
| 551 | PD-L1 CDR-H2 | GIIPIFGKAHYAQKFQG |
| 552 | PD-L1 CDR-H3 | KFHFVSGSPFGMDV |
| 553 | PD-L1 CDR-L1 | RASQSVSSYLA |
| 554 | PD-L1 CDR-L2 | DASNRAT |
| 555 | PD-L1 CDR-L3 | QQRSNWPT |
| 556 | PD-L1 VH | QVQLVQSGAEVKKPGSSVKVSCKTSGDTFSTAAISWVRQAPGQG |
| | | LEWMGGIIPIFGKAHYAQKFQGRVTITADESTSTAYMELSSLRSED |
| | | TAVYFCARKFHFVSGSPFGMDVWGQGTTVTVSS |
| 557 | PD-L1 VL | EIVLTQSPATLSLSPGERATLSCRASQSVSSYLAWYQQKPGQAPRL |
| | | LIYDASNRATGIPA |
| | | RFSGSGSGTDFTLTISSLEPEDFAVYYCQQRSNWPTFGQGTKVEIK |
| 558 | IGF-1R CDR-H1 | SYAIS |
| 559 | IGF-IR CDR-H2 | GIIPIFGTANYAQKFQG |
| 560 | IGF-1R CDR-H3 | APLRFLEWSTQDHYYYYYMDV |
| 561 | IGF-1R CDR-L1 | QGDSLRSYYAT |
| 562 | IGF-1R CDR-L2 | GENKRPS |
| 563 | IGF-1R CDR-L3 | KSRDGSGQHLV |
| 564 | IGF-1R VH | EVQLVQSGAEVKKPGSSVKVSCKASGGTFSSYAISWVRQAPGQGL |
| | | EWMGGIIPIFGTANY |
| | | AQKFQGRVTITADKSTSTAYMELSSLRSEDTAVYYCARAPLRFLE |
| | | WSTQDHYYYYYMDVWGKGTTVTVSS |
| 565 | IGF-1R VL | SSELTQDPAVSVALGQTVRITCQGDSLRSYYATWYQQKPGQAPIL |
| | | VIYGENKRPSGIPDR |
| | | FSGSSSGNTASLTITGAQAEDEADYYCKSRDGSGQHLVFGGGTKL |
| | | TVL |
| 566 | Claudin-18.2 CDR-H1 | SYWIN |
| 567 | Claudin-18.2 CDR-H2 | NIYPSDSYTNYNQKFKD |
| 568 | Claudin-18.2 CDR-H3 | SWRGNSFDY |
| 569 | Claudin-18.2 CDR-L1 | KSSQSLLNSGNQKNYLT |
| 570 | Claudin-18.2 CDR-L2 | WASTRES |
| 571 | Claudin-18.2 CDR-L3 | QNDYSYPFT |
| 572 | Claudin-18.2 VH | QVQLQQPGAELVRPGASVKLSCKASGYTFTSYWINWVKQRPGQG |
| | | LEWIGNIYPSDSYTN |
| | | YNQKFKDKATLTVDKSSSTAYMQLSSPTSEDSAVYYCTRSWRGN |
| | | SFDYWGQGTTLTVSS |
| 573 | Claudin-18.2 VL | DIVMTQSPSSLTVTAGEKVTMSCKSSQSLLNSGNQKNYLTWYQQ |
| | | KPGQPPKLLIYWASTR |
| | | ESGVPDRFTGSGSGTDFTLTISSVQAEDLAVYYCQNDYSYPFTFGS |
| | | GTKLEIK |
| 574 | Claudin-18.2 CDR-H1 | NYGMN |
| 575 | Claudin-18.2 CDR-H2 | WINTNTGEPTYAEEFKG |
| 576 | Claudin-18.2 CDR-H3 | LGFGNAMDY |
| 577 | Claudin-18.2 CDR-L1 | KSSQSLLNSGNQKNYLT |
| 578 | Claudin-18.2 CDR-L2 | WASTRES |
| 579 | Claudin-18.2 CDR-L3 | QNDYSYPLT |
| 580 | Claudin-18.2 VH | QIQLVQSGPELKKPGETVKISCKASGYTFTNYGMNWVKQAPGKG |
| | | LKWMGWINTNTGEPTY |
| | | AEEFKGRFAFSLETSASTAYLQINNLKNEDTATYFCARLGFGNAM |
| | | DYWGQGTSVTVSS |
| 581 | Claudin-18.2 VL | DIVMTQSPSSLTVTAGEKVTMSCKSSQSLLNSGNQKNYLTWYQQ |
| | | KPGQPPKLLIYWASTR |
| | | ESGVPDRFTGSGSGTDFTLTISSVQAEDLAVYYCQNDYSYPLTFGA |
| | | GTKLELK |
| 582 | Nectin-4 CDR-H1 | SYNMN |
| 583 | Nectin-4 CDR-H2 | YISSSSSTIYYADSVKG |
| 584 | Nectin-4 CDR-H3 | AYYYGMDV |
| 585 | Nectin-4 CDR-L1 | RASQGISGWLA |
| 586 | Nectin-4 CDR-L2 | AASTLQS |
| 587 | Nectin-4 CDR-L3 | QQANSFPPT |
| 588 | Nectin-4 VH | EVQLVESGGGLVQPGGSLRLSCAASGFTFSSYNMNWVRQAPGKG |
| | | LEWVSYISSSSSTIYY |
| | | ADSVKGRFTISRDNAKNSLSLQMNSLRDEDTAVYYCARAYYYGM |
| | | DVWGQGTTVTVSS |
| 589 | Nectin-4 VL | DIQMTQSPSSVSASVGDRVTITCRASQGISGWLAWYQQKPGKAPK |
| | | FLIYAASTLQSGVPS |
| | | RFSGSGSGTDFTLTISSLQPEDFATYYCQQANSFPPTFGGGTKVEIK |
| 590 | SLTRK6 CDR-H1 | SYGMH |
| 591 | SLTRK6 CDR-H2 | VIWYDGSNQYYADSVKG |
| 592 | SLTRK6 CDR-H3 | GLTSGRYGMDV |
| 593 | SLTRK6 CDR-L1 | RSSQSLLLSHGFNYLD |
| 594 | SLTRK6 CDR-L2 | LGSSRAS |
| 595 | SLTRK6 CDR-L3 | MQPLQIPWT |
| 596 | SLTRK6 VH | QVQLVESGGGVVQPGRSLRLSCAASGFTFSSYGMHWVRQAPGKG |
| | | LEWVAVIWYDGSNQYY |
| | | ADSVKGRFTISRDNSKNTLFLQMHSLRAEDTAVYYCARGLTSGRY |
| | | GMDVWGQGTTVTVSS |
| 597 | SLTRK6 VL | DIVMTQSPLSLPVTPGEPASISCRSSQSLLLSHGFNYLDWYLQKPG |
| | | QSPQLLIYLGSSRASGVPDRFSGSGSGTDFTLKISRVEAEDVGLYY |
| | | CMQPLQIPWTFGQGTKVEIK |
| 598 | CD228 CDR-H1 | SGYWN |
| 599 | CD228 CDR-H2 | YISDSGITYYNPSLKS |
| 600 | CD228 CDR-H3 | RTLATYYAMDY |
| 601 | CD228 CDR-L1 | RASQSLVHSDGNTYLH |
| 602 | CD228 CDR-L2 | RVSNRFS |
| 603 | CD228 CDR-L3 | SQSTHVPPT |
| 604 | CD228 VH | QVQLQESGPGLVKPSETLSLTCTVSGDSITSGYWNWIRQPPGKGLE |
| | | YIGYISDSGITYYN |
| | | PSLKSRVTISRDTSKNQYSLKLSSVTAADTAVYYCARRTLATYYA |
| | | MDYWGQGTLVTVSS |
| 605 | CD228 VL | DFVMTQSPLSLPVTLGQPASISCRASQSLVHSDGNTYLHWYQQRP |
| | | GQSPRLLIYRVSNRFSGVPDRFSGSGSGTDFTLKISRVEAEDVGVY |
| | | YCSQSTHVPPTFGQGTKLEIKR |
| 606 | CD142 (TF) CDR-H1 | NYAMS |
| 607 | CD142 (TF) CDR-H2 | SISGSGDYTYYTDSVKG |
| 608 | CD142 (TF) CDR-H3 | SPWGYYLDS |
| 609 | CD142 (TF) CDR-L1 | RASQGISSRLA |
| 610 | CD142 (TF) CDR-L2 | AASSLQS |
| 611 | CD142 (TF) CDR-L3 | QQYNSYPYT |
| 612 | CD142 (TF) VH | EVQLLESGGGLVQPGGSLRLSCAASGFTFSNYAMSWVRQAPGKG |
| | | LEWVSSISGSGDYTY |
| | | YTDSVKGRFTISRDNSKNTLYLQMNSLRAEDTAVYYCARSPWGY |
| | | YLDSWGQGTLVTVSS |
| 613 | CD142 (TF) VL | DIQMTQSPPSLSASAGDRVTITCRASQGISSRLAWYQQKPEKAPKS |
| | | LIYAASSLQSGVPS |
| | | RFSGSGSGTDFTLTISSLQPEDFATYYCQQYNSYPYTFGQGTKLEIK |
| 614 | STn CDR-H1 | DHAIH |
| 615 | STn CDR-H2 | YFSPGNDDIKYNEKFRG |
| 616 | STn CDR-H3 | SLSTPY |
| 617 | STn CDR-L1 | KSSQSLLNRGNHKNYLT |
| 618 | STn CDR-L2 | WASTRES |
| 619 | STn CDR-L3 | QNDYTYPYT |
| 620 | STn VH | EVQLVQSGAEVKKPGASVKVSCKASGYTFTDHAIHWVRQAPGQG |
| | | LEWMGYFSPGNDDIKY |
| | | NEKFRGRVTMTADKSSSTAYMELRSLRSDDTAVYFCKRSLSTPY |
| | | WGQGTLVTVSS |
| 621 | STn VL | DIVMTQSPDSLAVSLGERATINCKSSQSLLNRGNHKNYLTWYQQK |
| | | PGQPPKLLIYWAST |
| | | RESGVPDRFSGSGSGTDFTLTISSLQAEDVAVYYCQNDYTYPYTFG |
| | | QGTKVEIK |
| 622 | CD20 CDR-H1 | SYNMH |
| 623 | CD20 CDR-H2 | AIYPGNGDTSYNQKFKG |
| 624 | CD20 CDR-H3 | STYYGGDWYFNV |
| 625 | CD20 CDR-L1 | RASSSVSYIH |
| 626 | CD20 CDR-L2 | ATSNLAS |
| 627 | CD20 CDR-L3 | QQWTSNPPT |
| 628 | CD20 VH | QVQLQQPGAELVKPGASVKMSCKASGYTFTSYNMHWVKQTPGR |
| | | GLEWIGAIYPGNGDTSY |
| | | NQKFKGKATLTADKSSSTAYMQLSSLTSEDSAVYYCARSTYYGG |
| | | DWYFNVWGAGTTVTVSA |
| 629 | CD20 VL | QIVLSQSPAILSASPGEKVTMTCRASSSVSYIHWFQQKPGSSPKPWI |
| | | YATSNLASGVPVR |
| | | FSGSGSGTSYSLTISRVEAEDAATYYCQQWTSNPPTFGGGTKLEIK |
| 630 | HER2 CDR-H1 | DTYIH |
| 631 | HER2 CDR-H2 | RIYPTNGYTRYADSVKG |
| 632 | HER2 CDR-H3 | WGGDGFYAMDY |
| 633 | HER2 CDR-L1 | RASQDVNTAVA |
| 634 | HER2 CDR-L2 | SASFLYS |
| 635 | HER2 CDR-L3 | QQHYTTPPT |
| 636 | HER2 VH | EVQLVESGGGLVQPGGSLRLSCAASGFNIKDTYIHWVRQAPGKGL |
| | | EWVARIYPTNGYTRY |
| | | ADSVKGRFTISADTSKNTAYLQMNSLRAEDTAVYYCSRWGGDGF |
| | | YAMDYWGQGTLVTVSS |
| 637 | HER2 VL | DIQMTQSPSSLSASVGDRVTITCRASQDVNTAVAWYQQKPGKAP |
| | | KLLIYSASFLYSGVPS |
| | | RFSGSRSGTDFTLTISSLQPEDFATYYCQQHYTTPPTFGQGTKVEIK |
| 638 | CD79b CDR-H1 | SYWIE |
| 639 | CD79b CDR-H2 | EILPGGGDTNYNEIFKG |
| 640 | CD79b CDR-H3 | RVPIRLDY |
| 641 | CD79b CDR-L1 | KASQSVDYEGDSFLN |
| 642 | CD79b CDR-L2 | AASNLES |
| 643 | CD79b CDR-L3 | QQSNEDPLT |
| 644 | CD79b VH | EVQLVESGGGLVQPGGSLRLSCAASGYTFSSYWIEWVRQAPGKG |
| | | LEWIGEILPGGGDTNYNEIFKGRATFSADTSKNTAYLQMNSLRAE |
| | | DTAVYYCTRRVPIRLDYWGQGTLVTVSS |
| 645 | CD79b VL | DIQLTQSPSSLSASVGDRVTITCKASQSVDYEGDSFLNWYQQKPG |
| | | KAPKLLIYAASNLES |
| | | GVPSRFSGSGSGTDFTLTISSLQPEDFATYYCQQSNEDPLTFGQGT |
| | | KVEIK |
| 646 | NaPi2B CDR-H1 | DFAMS |
| 647 | NaPi2B CDR-H2 | TIGRVAFHTYYPDSMKG |
| 648 | NaPi2B CDR-H3 | HRGFDVGHFDF |
| 649 | NaPi2B CDR-L1 | RSSETLVHSSGNTYLE |
| 650 | NaPi2B CDR-L2 | RVSNRFS |
| 651 | NaPi2B CDR-L3 | FQGSFNPLT |
| 652 | NaPi2B VH | EVQLVESGGGLVQPGGSLRLSCAASGFSFSDFAMSWVRQAPGKG |
| | | LEWVATIGRVAFHTYY |
| | | PDSMKGRFTISRDNSKNTLYLQMNSLRAEDTAVYYCARHRGFDV |
| | | GHFDFWGQGTLVTVSS |
| 653 | NaPi2B VL | DIQMTQSPSSLSASVGDRVTITCRSSETLVHSSGNTYLEWYQQKPG |
| | | KAPKLLIYRVSNRF |
| | | SGVPSRFSGSGSGTDFTLTISSLQPEDFATYYCFQGSFNPLTFGQGT |
| | | KVEIK |
| 654 | Muc16 CDR-H1 | NDYAWN |
| 655 | Muc16 CDR-H2 | YISYSGYTTYNPSLKS |
| 656 | Muc16 CDR-H3 | WTSGLDY |
| 657 | Muc16 CDR-L1 | KASDLIHNWLA |
| 658 | Muc16 CDR-L2 | GATSLET |
| 659 | Muc16 CDR-L3 | QQYWTTPFT |
| 660 | Muc16 VH | EVQLVESGGGLVQPGGSLRLSCAASGYSITNDYAWNWVRQAPGK |
| | | GLEWVGYISYSGYTTY |
| | | NPSLKSRFTISRDTSKNTLYLQMNSLRAEDTAVYYCARWTSGLDY |
| | | WGQGTLVTVSS |
| 661 | Muc16 VL | DIQMTQSPSSLSASVGDRVTITCKASDLIHNWLAWYQQKPGKAPK |
| | | LLIYGATSLETGVPSRFSGSGSGTDFTLTISSLQPEDFATYYCQQYW |
| | | TTPFTFGQGTKVEIK |
| 662 | STEAP1 CDR-H1 | SDYAWN |
| 663 | STEAP1 CDR-H2 | YISNSGSTSYNPSLKS |
| 664 | STEAP1 CDR-H3 | ERNYDYDDYYYAMDY |
| 665 | STEAP1 CDR-L1 | KSSQSLLYRSNQKNYLA |
| 666 | STEAP1 CDR-L2 | WASTRES |
| 667 | STEAP1 CDR-L3 | QQYYNYPRT |
| 668 | STEAP1 VH | EVQLVESGGGLVQPGGSLRLSCAVSGYSITSDYAWNWVRQAPGK |
| | | GLEWVGYISNSGSTSYNPSLKSRFTISRDTSKNTLYLQMNSLRAED |
| | | TAVYYCARERNYDYDDYYYAMDYWGQGTLVTVSS |
| 669 | STEAP1 VL | DIQMTQSPSSLSASVGDRVTITCKSSQSLLYRSNQKNYLAWYQQK |
| | | PGKAPKLLIYWASTRESGVPSRFSGSGSGTDFTLTISSLQPEDFATY |
| | | YCQQYYNYPRTFGQGTKVEIK |
| 670 | BCMA CDR-H1 | NYWMH |
| 671 | BCMA CDR-H2 | ATYRGHSDTYYNQKFKG |
| 672 | BCMA CDR-H3 | GAIYDGYDVLDN |
| 673 | BCMA CDR-L1 | SASQDISNYLN |
| 674 | BCMA CDR-L2 | YTSNLHS |
| 675 | BCMA CDR-L3 | QQYRKLPWT |
| 676 | BCMA VH | QVQLVQSGAEVKKPGSSVKVSCKASGGTFSNYWMHWVRQAPGQ |
| | | GLEWMGATYRGHSDTYYNQKFKGRVTITADKSTSTAYMELSSLR |
| | | SEDTAVYYCARGAIYDGYDVLDNWGQGTLVTVSS |
| 677 | BCMA VL | DIQMTQSPSSLSASVGDRVTITCSASQDISNYLNWYQQKPGKAPKL |
| | | LIYYTSNLHSGVPSRFSGSGSGTDFTLTISSLQPEDFATYYCQQYRK |
| | | LPWTFGQGTKLEIK |
| 678 | c-Met CDR-H1 | AYTMH |
| 679 | c-Met CDR-H2 | WIKPNNGLANYAQKFQG |
| 680 | c-Met CDR-H3 | SEITTEFDY |
| 681 | c-Met CDR-L1 | KSSESVDSYANSFLH |
| 682 | c-Met CDR-L2 | RASTRES |
| 683 | c-Met CDR-L3 | QQSKEDPLT |
| 684 | c-Met VH | QVQLVQSGAEVKKPGASVKVSCKASGYIFTAYTMHWVRQAPGQ |
| | | GLEWMGWIKPNNGLAN |
| | | YAQKFQGRVTMTRDTSISTAYMELSRLRSDDTAVYYCARSEITTE |
| | | FDYWGQGTLVTVSS |
| 685 | c-Met VL | DIVMTQSPDSLAVSLGERATINCKSSESVDSYANSFLHWYQQKPG |
| | | QPPKLLIYRASTRE |
| | | SGVPDRFSGSGSGTDFTLTISSLQAEDVAVYYCQQSKEDPLTFGGG |
| | | TKVEIK |
| 686 | EGFR CDR-H1 | SDFAWN |
| 687 | EGFR CDR-H2 | YISYSGNTRYQPSLKS |
| 688 | EGFR CDR-H3 | AGRGFPY |
| 689 | EGFR CDR-L1 | HSSQDINSNIG |
| 690 | EGFR CDR-L2 | HGTNLDD |
| 691 | EGFR CDR-L3 | VQYAQFPWT |
| 692 | EGFR VH | QVQLQESGPGLVKPSQTLSLTCTVSGYSISSDFAWNWIRQPPGKGL |
| | | EWMGYISYSGNTRY |
| | | QPSLKSRITISRDTSKNQFFLKLNSVTAADTATYYCVTAGRGFPYW |
| | | GQGTLVTVSS |
| 693 | EGFR VL | DIQMTQSPSSMSVSVGDRVTITCHSSQDINSNIGWLQQKPGKSFKG |
| | | LIYHGTNLDDGVPS |
| | | RFSGSGSGTDYTLTISSLQPEDFATYYCVQYAQFPWTFGGGTKLEI |
| | | K |
| 694 | SLAMF7 CDR-H1 | DYYMA |
| 695 | SLAMF7 CDR-H2 | SINYDGSSTYYVDSVKG |
| 696 | SLAMF7 CDR-H3 | DRGYYFDY |
| 697 | SLAMF7 CDR-L1 | RSSQSLVHSNGNTYLH |
| 698 | SLAMF7 CDR-L2 | KVSNRFS |
| 699 | SLAMF7 CDR-L3 | SQSTHVPPFT |
| 700 | SLAMF7 VH | EVQLVESGGGLVQPGGSLRLSCAASGFTFSDYYMAWVRQAPGKG |
| | | LEWVASINYDGSSTY |
| | | YVDSVKGRFTISRDNAKNSLYLQMNSLRAEDTAVYYCARDRGYY |
| | | FDYWGQGTTVTVSS |
| 701 | SLAMF7 VL | DVVMTQTPLSLSVTPGQPASISCRSSQSLVHSNGNTYLHWYLQKP |
| | | GQSPQLLIYKVSNRF |
| | | SGVPDRFSGSGSGTDFTLKISRVEAEDVGVYFCSQSTHVPPFTFGG |
| | | GTKVEIK |
| 702 | SLITRK6 CDR-H1 | SYGMH |
| 703 | SLITRK6 CDR-H2 | VIWYDGSNQYYADSVKG |
| 704 | SLITRK6 CDR-H3 | GLTSGRYGMDV |
| 705 | SLITRK6 CDR-L1 | RSSQSLLLSHGFNYLD |
| 706 | SLITRK6 CDR-L2 | LGSSRAS |
| 707 | SLITRK6 CDR-L3 | MQPLQIPWT |
| 708 | SLITRK6 VH | QVQLVESGGGVVQPGRSLRLSCAASGFTFSSYGMHWVRQAPGKG |
| | | LEWVAVIWYDGSNQYY |
| | | ADSVKGRFTISRDNSKNTLFLQMHSLRAEDTAVYYCARGLTSGRY |
| | | GMDVWGQGTTVTVSS |
| 709 | SLITRK6 VL | DIVMTQSPLSLPVTPGEPASISCRSSQSLLLSHGFNYLDWYLQKPG |
| | | QSPQLLIYLGSSRA |
| | | SGVPDRFSGSGSGTDFTLKISRVEAEDVGLYYCMQPLQIPWTFGQ |
| | | GTKVEIK |
| 710 | C4.4a CDR-H1 | NAWMS |
| 711 | C4.4a CDR-H2 | YISSSGSTIYYADSVKG |
| 712 | C4.4a CDR-H3 | EGLWAFDY |
| 713 | C4.4a CDR-L1 | TGSSSNIGAGYVVH |
| 714 | C4.4a CDR-L2 | DNNKRPS |
| 715 | C4.4a CDR-L3 | AAWDDRLNGPV |
| 716 | C4.4a VH | EVQLLESGGGLVQPGGSLRLSCAASGFTFSNAWMSWVRQAPGKG |
| | | LEWVSYISSSGSTIYY |
| | | ADSVKGRFTISRDNSKNTLYLQMNSLRAEDTAVYYCAREGLWAF |
| | | DYWGQGTLVTVSS |
| 717 | C4.4a VL | ESVLTQPPSVSGAPGQRVTISCTGSSSNIGAGYVVHWYQQLPGTAP |
| | | KLLIYDNNKRPSGV |
| | | PDRFSGSKSGTSASLAISGLRSEDEADYYCAAWDDRLNGPVFGGG |
| | | TKLTVL |
| 718 | GCC CDR-H1 | GYYWS |
| 719 | GCC CDR-H2 | EINHRGNTNDNPSLKS |
| 720 | GCC CDR-H3 | ERGYTYGNFDH |
| 721 | GCC CDR-L1 | RASQSVSRNLA |
| 722 | GCC CDR-L2 | GASTRAT |
| 723 | GCC CDR-L3 | QQYKTWPRT |
| 724 | GCC VH | QVQLQQWGAGLLKPSETLSLTCAVFGGSFSGYYWSWIRQPPGKG |
| | | LEWIGEINHRGNTNDN |
| | | PSLKSRVTISVDTSKNQFALKLSSVTAADTAVYYCARERGYTYGN |
| | | FDHWGQGTLVTVSS |
| 725 | GCC VL | EIVMTQSPATLSVSPGERATLSCRASQSVSRNLAWYQQKPGQAPR |
| | | LLIYGASTRATGIP |
| | | ARFSGSGSGTEFTLTIGSLQSEDFAVYYCQQYKTWPRTFGQGTNV |
| | | EIK |
| 726 | Axl CDR-H1 | SYAMN |
| 727 | Axl CDR-H2 | TTSGSGASTYYADSVKG |
| 728 | Axl CDR-H3 | IWIAFDI |
| 729 | Axl CDR-L1 | RASQSVSSSYLA |
| 730 | Axl CDR-L2 | GASSRAT |
| 731 | Axl CDR-L3 | QQYGSSPYT |
| 732 | Axl VH | EVQLLESGGGLVQPGGSLRLSCAASGFTFSSYAMNWVRQAPGKG |
| | | LEWVSTTSGSGASTYY |
| | | ADSVKGRFTISRDNSKNTLYLQMNSLRAEDTAVYYCAKIWIAFDI |
| | | WGQGTMVTVSS |
| 733 | Axl VL | EIVLTQSPGTLSLSPGERATLSCRASQSVSSSYLAWYQQKPGQAPR |
| | | LLIYGASSRATGIP |
| | | DRFSGSGSGTDFTLTISRLEPEDFAVYYCQQYGSSPYTFGQGTKLEI |
| | | K |
| 734 | gpNMB CDR-H1 | SFNYYWS |
| 735 | gpNMB CDR-H2 | YIYYSGSTYSNPSLKS |
| 736 | gpNMB CDR-H3 | GYNWNYFDY |
| 737 | gpNMB CDR-L1 | RASQSVDNNLV |
| 738 | gpNMB CDR-L2 | GASTRAT |
| 739 | gpNMB CDR-L3 | QQYNNWPPWT |
| 740 | gpNMB VH | QVQLQESGPGLVKPSQTLSLTCTVSGGSISSFNYYWSWIRHHPGK |
| | | GLEWIGYTYYSGSTY |
| | | SNPSLKSRVTISYDTSKNQFSLTLSSVTAADTAVYYCARGYNWNY |
| | | FDYWGQGTLVTVSS |
| 741 | gpNMB VL | EIVMTQSPATLSVSPGERATLSCRASQSVDNNLVWYQQKPGQAPR |
| | | LLIYGASTRATGIPA |
| | | RFSGSGSGTEFTLTISSLQSEDFAVYYCQQYNNWPPWTFGQGTKV |
| | | EIK |
| 742 | Prolactin receptor | TYWMH |
| | CDR-H1 | |
| 743 | Prolactin receptor | EIDPSDSYSNYNQKFKD |
| | CDR-H2 | |
| 744 | Prolactin receptor | NGGLGPAWFSY |
| | CDR-H3 | |
| 745 | Prolactin receptor | KASQYVGTAVA |
| | CDR-L1 | |
| 746 | Prolactin receptor | SASNRYT |
| | CDR-L2 | |
| 747 | Prolactin receptor | QQYSSYPWT |
| | CDR-L3 | |
| 748 | Prolactin receptor VH | EVQLVQSGAEVKKPGSSVKVSCKASGYTFTTYWMHWVRQAPGQ |
| | | GLEWIGEIDPSDSYSNY |
| | | NQKFKDRATLTVDKSTSTAYMELSSLRSEDTAVYYCARNGGLGP |
| | | AWFSYWGQGTLVTVSS |
| 749 | Prolactin receptor VL | DIQMTQSPSSVSASVGDRVTITCKASQYVGTAVAWYQQKPGKSP |
| | | KLLIYSASNRYTGVPS |
| | | RFSDSGSGTDFTLTISSLQPEDFATYFCQQYSSYPWTFGGGTKVEIK |
| 750 | FGFR2 CDR-H1 | SYAMS |
| 751 | FGFR2 CDR-H2 | AISGSGTSTYYADSVKG |
| 752 | FGFR2 CDR-H3 | VRYNWNHGDWFDP |
| 753 | FGFR2 CDR-L1 | SGSSSNIGNNYVS |
| 754 | FGFR2 CDR-L2 | ENYNRPA |
| 755 | FGFR2 CDR-L3 | SSWDDSLNYWV |
| 756 | FGFR2 VH | EVQLLESGGGLVQPGGSLRLSCAASGFTFSSYAMSWVRQAPGKG |
| | | LEWVSAISGSGTSTYYADSVKGRFTISRDNSKNTLYLQMNSLRAE |
| | | DTAVYYCARVRYNWNHGDWFDPWGQGTLVTVSS |
| 757 | FGFR2 VL | QSVLTQPPSASGTPGQRVTISCSGSSSNIGNNYVSWYQQLPGTAPK |
| | | LLIYENYNRPAGVP |
| | | DRFSGSKSGTSASLAISGLRSEDEADYYCSSWDDSLNYWVFGGGT |
| | | KLTVL |
| 758 | CDCP1 CDR-H1 | SYGMS |
| 759 | CDCP1 CDR-H2 | TISSGGSYKYYVDSVKG |
| 760 | CDCP1 CDR-H3 | HPDYDGVWFAY |
| 761 | CDCP1 CDR-L1 | SVSSSVFYVH |
| 762 | CDCP1 CDR-L2 | DTSKLAS |
| 763 | CDCP1 CDR-L3 | QQWNSNPPT |
| 764 | CDCP1 VH | EVQLVESGGGLVQPGGSLRLSCAASGFTFNSYGMSWVRQAPGKG |
| | | LEWVATISSGGSYKYY |
| | | VDSVKGRFTISRDNAKNSLYLQMNSLRAEDTAVYYCARHPDYDG |
| | | VWFAYWGQGTLVTVSS |
| 765 | CDCP1 VL | DIQMTQSPSSLSASVGDRVTITCSVSSSVFYVHWYQQKPGKAPKL |
| | | LIYDTSKLASSGVPS |
| | | RFSGSGSGTDFTFTISSLQPEDIATYYCQQWNSNPPTFGGGTKVEIK |
| 766 | CDCP1 CDR-H1 | SYGMS |
| 767 | CDCP1 CDR-H2 | TISSGGSYTYYPDSVKG |
| 768 | CDCP1 CDR-H3 | HPDYDGVWFAY |
| 769 | CDCP1 CDR-L1 | SVSSSVFYVH |
| 770 | CDCP1 CDR-L2 | DTSKLAS |
| 771 | CDCP1 CDR-L3 | QQWNSNPPT |
| 772 | CDCP1 VH | EVQLVESGGDLVKPGGSLKLSCAASGFTFNSYGMSWVRQTPDKR |
| | | LEWVATISSGGSYTYY |
| | | PDSVKGRFTISRDNAKNTLYLQMSSLKSEDTAMYYCARHPDYDG |
| | | VWFAYWGQGTLVTVSA |
| 773 | CDCP1 VL | QIVLTQSPAIMASPGEKVTMTCSVSSSVFYVHWYQQKSGTSPKRW |
| | | IYDTSKLASGVPARF |
| | | SGSGSGTSYSLTISSMEAEDAATYYCQQWNSNPPTFGGGTKLEIK |
| 774 | CDCP1 CDR-H1 | SYYMH |
| 775 | CDCP1 CDR-H2 | IINPSGGSTSYAQKFQG |
| 776 | CDCP1 CDR-H3 | DGVLRYFDWLLDYYYY |
| 777 | CDCP1 CDR-L1 | RASQSVGSYLA |
| 778 | CDCP1 CDR-L2 | DASNRAT |
| 779 | CDCP1 CDR-L3 | QQRANVFT |
| 780 | CDCP1 VH | EVQLVQSGAEVKKPGASVKVSCKASGYTFTSYYMHWVRQAPGQ |
| | | GLEWMGIINPSGGSTSY |
| | | AQKFQGRVTMTRDTSTSTVYMELSSLRSEDTAVYYCARDGVLRY |
| | | FDWLLDYYYYMDVWGKG |
| | | TTVTVSS |
| 781 | CDCP1 VL | EIVLTQSPATLSLSPGERATLSCRASQSVGSYLAWYQQRPGQAPRL |
| | | LIYDASNRATGIPA |
| | | RFSGSGSGTDFTLTISSLEPEDFAVYYCQQRANVFTFGQGTKVEIK |
| 782 | CDCP1 CDR-H1 | SYYMH |
| 783 | CDCP1 CDR-H2 | IINPSGGSTSYAQKFQG |
| 784 | CDCP1 CDR-H3 | DAELRHFDHLLDYHYYMDV |
| 785 | CDCP1 CDR-L1 | RASQSVGSYLA |
| 786 | CDCP1 CDR-L2 | DASNRAT |
| 787 | CDCP1 CDR-L3 | QQRAQEFT |
| 788 | CDCP1 VH | EVQLVQSGAEVKKPGASVKVSCKASGYTFTSYYMHWVRQAPGQ |
| | | GLEWMGIINPSGGSTSYAQKFQGRVTMTRDTSTSTVYMELSSLRS |
| | | EDTAVYYCARDAELRHFDHLLDYHYYMDVWGQGTTVTVSS |
| 789 | CDCP1 VL | EIVMTQSPATLSLSPGERATLSCRASQSVGSYLAWYQQKPGQAPR |
| | | LLIYDASNRATGIPA |
| | | RFSGSGSGTDFTLTISSLQPEDFAVYYCQQRAQEFTFGQGTKVEIK |
| 790 | ASCT2 VH | QVQLVQSGSELKKPGAPVKVSCKASGYTFSTFGMSWVRQAPGQG |
| | | LKWMGWIHTYAGVPIYGDDFKGRFVFSLDTSVSTAYLQISSLKAE |
| | | DTAVYFCARRSDNYRYFFDYWGQGTTVTVSS |
| 791 | ASCT2 VL | DIQMTQSPSSLSASLGDRVTITCRASQDIRNYLNWYQQKPGKAPK |
| | | LLIYYTSRLHSGVPSRFSGSGSGTDYTLTISSLQPEDFATYFCQQGH |
| | | TLPPTFGQGTKLEIK |
| 792 | ASCT2 VH | QIQLVQSGPELKKPGAPVKISCKASGYTFTTFGMSWVKQAPGQGL |
| | | KWMGWIHTYAGVPIYGDDFKGRFVFSLDTSVSTAYLQISSVKAED |
| | | TATYFCARRSDNYRYFFDYWGQGTTLTVSS |
| 793 | ASCT2 VL | DIQMTQSPSSLSASLGDRVTITCRASQDIRNYLNWYQQKPGKAPK |
| | | LLIYYTSRLHSGVPS |
| | | RFSGSGSGTDYTLTISSLQPEDFATYFCQQGHTLPPTFGQGTKLEIK |
| 794 | ASCT2 CDR-H1 | NYYMA |
| 795 | ASCT2 CDR-H2 | SITKGGGNTYYRDSVKG |
| 796 | ASCT2 CDR-H3 | QVTIAAVSTSYFDS |
| 797 | ASCT2 CDR-L1 | KTNQKVDYYGNSYVY |
| 798 | ASCT2 CDR-L2 | LASNLAS |
| 799 | ASCT2 CDR-L3 | QQSRNLPYT |
| 800 | ASCT2 VH | EVQLVESGGGLVQSGRSIRLSCAASGFSFSNYYMAWVRQAPSKGL |
| | | EWVASITKGGGNTYYRDSVKGRFTFSRDNAKSTLYLQMDSLRSE |
| | | DTATYYCARQVTIAAVSTSYFDSWGQGVMVTVSS |
| 801 | ASCT2 VL | DIVLTQSPALAVSLGQRATISCKTNQKVDYYGNSYVYWYQQKPG |
| | | QQPKLLIYLASNLASGIPARFSGRGSGTDFTLTIDPVEADDTATYY |
| | | CQQSRNLPYTFGAGTKLELK |
| 802 | CD123 CDR-H1 | DYYMK |
| 803 | CD123 CDR-H2 | DIIPSNGATFYNQKFKG |
| 804 | CD123 CDR-H3 | SHLLRASWFAY |
| 805 | CD123 CDR-L1 | KSSQSLLNSGNQKNYLT |
| 806 | CD123 CDR-L2 | WASTRES |
| 807 | CD123 CDR-L3 | QNDYSYPYT |
| 808 | CD123 VH | QVQLVQSGAEVKKPGASVKMSCKASGYTFTDYYMKWVKQAPG |
| | | QGLEWIGDIIPSNGATFYNQKFKGKATLTVDRSISTAYMHLNRLRS |
| | | DDTAVYYCTRSHLLRASWFAYWGQGTLVTVSS |
| 809 | CD123 VL | DFVMTQSPDSLAVSLGERATINCKSSQSLLNSGNQKNYLTWYLQK |
| | | PGQPPKLLIYWASTRESGVPDRFSGSGSGTDFTLTISSLQAEDVAV |
| | | YYCQNDYSYPYTFGQGTKLEIK |
| 810 | GPC3 CDR-H1 | DYEMH |
| 811 | GPC3 CDR-H2 | WIGGIDPETGGTAYNQKFKG |
| 812 | GPC3 CDR-H3 | YYSFAY |
| 813 | GPC3 CDR-L1 | RSSQSIVHSNGNTYLQ |
| 814 | GPC3 CDR-L2 | KVSNRFS |
| 815 | GPC3 CDR-L3 | FQVSHVPYT |
| 816 | GPC3 VH | EVQLVQSGAEVKKPGATVKISCKVSGYTFTDYEMHWVQQAPGK |
| | | GLEWMGGIDPETGGTAYNQKFKGRVTLTADKSTDTAYMELSSLR |
| | | SEDTAVYYCGRYYSFAYWGQGTLVTVSS |
| 817 | GPC3 VL | DVVMTQSPLSLPVTLGQPASISCRSSQSIVHSNANTYLQWFQQRPG |
| | | QSPRLLIYKVSNRFSGVPDRFSGSGSGTDFTLKISRVEAEDVGVYY |
| | | CFQVSHVPYTFGQGTKLEIK |
| 818 | B6A CDR-H1 | DYNVN |
| 819 | B6A CDR-H2 | VINPKYGTTRYNQKFKG |
| 820 | B6A CDR-H3 | GLNAWDY |
| 821 | B6A CDR-L1 | GASENIYGALN |
| 822 | B6A CDR-L2 | GATNLED |
| 823 | B6A CDR-L3 | QNVLTTPYT |
| 824 | B6A VH | QFQLVQSGAEVKKPGASVKVSCKASGYSFTDYNVNWVRQAPGQ |
| | | GLEWIGVINPKYGTTRYNQKFKGRATLTVDKSTSTAYMELSSLRS |
| | | EDTAVYYCTRGLNAWDYWGQGTLVTVSS |
| 825 | B6A VL | DIQMTQSPSSLSASVGDRVTITCGASENIYGALNWYQQKPGKAPK |
| | | LLIYGATNLEDGVPSRFSGSGSGRDYTFTISSLQPEDIATYYCQNVL |
| | | TTPYTFGQGTKLEIK |
| 826 | B6A CDR-H1 | GYFMN |
| 827 | B6A CDR-H2 | LINPYNGDSFYNQKFKG |
| 828 | B6A CDR-H3 | GLRRDFDY |
| 829 | B6A CDR-L1 | KSSQSLLDSDGKTYLN |
| 830 | B6A CDR-L2 | LVSELDS |
| 831 | B6A CDR-L3 | WQGTHFPRT |
| 832 | B6A VH | QVQLVQSGAEVKKPGASVKVSCKASGYSFSGYFMNWVRQAPGQ |
| | | GLEWMGLINPYNGDSFYNQKFKGRVTMTRQTSTSTVYMELSSLR |
| | | SEDTAVYYCVRGLRRDFDYWGQGTLVTVSS |
| 833 | B6A VL | DVVMTQSPLSLPVTLGQPASISCKSSQSLLDSDGKTYLNWLFQRPG |
| | | QSPRRLIYLVSELDSGVPDRFSGSGSGTDFTLKISRVEAEDVGVYY |
| | | CWQGTHFPRTFGGGTKLEIK |
| 834 | PD-L1 CDR-H1 | TAAIS |
| 835 | PD-L1 CDR-H2 | GIIPIFGKAHYAQKFQG |
| 836 | PD-L1 CDR-H3 | KFHFVSGSPFGMDV |
| 837 | PD-L1 CDR-L1 | RASQSVSSYLA |
| 838 | PD-L1 CDR-L2 | DASNRAT |
| 839 | PD-L1 CDR-L3 | QQRSNWPT |
| 840 | PD-L1 VH | QVQLVQSGAEVKKPGSSVKVSCKTSGDTFSTAAISWVRQAPGQG |
| | | LEWMGGIIPIFGKAHYAQKFQGRVTITADESTSTAYMELSSLRSED |
| | | TAVYFCARKFHFVSGSPFGMDVWGQGTTVTVSS |
| 841 | PD-L1 VL | EIVLTQSPATLSLSPGERATLSCRASQSVSSYLAWYQQKPGQAPRL |
| | | LIYDASNRATGIPARFSGSGSGTDFTLTISSLEPEDFAVYYCQQRSN |
| | | WPTFGQGTKVEIK |
| 842 | TIGIT CDR-H1 | GTFSSYAIS |
| 843 | TIGIT CDR-H2 | SIIPIFGTANYAQKFQG |
| 844 | TIGIT CDR-H3 | ARGPSEVGAILGYVWFDP |
| 845 | TIGIT CDR-L1 | RSSQSLLHSNGYNYLD |
| 846 | TIGIT CDR-L2 | LGSNRAS |
| 847 | TIGIT CDR-L3 | MQARRIPIT |
| 848 | TIGIT VH | QVQLVQSGAEVKKPGSSVKVSCKASGGTFSSYAISWVRQAPGQG |
| | | LEWMGSIIPIFGTANYAQKFQGRVTITADESTSTAYMELSSLRSED |
| | | TAVYYCARGPSEVGAILGYVWFDPWGQGTLVTVSS |
| 849 | TIGIT VL | DIVMTQSPLSLPVTPGEPASISCRSSQSLLHSNGYNYLDWYLQKPG |
| | | QSPQLLIYLGSNRASGVPDRFSGSGSGTDFTLKISRVEAEDVGVYY |
| | | CMQARRIPITFGGGTKVEIK |
| 850 | STN CDR-H1 | GYTFTDHAIHWV |
| 851 | STN CDR-H2 | FSPGNDDIKY |
| 852 | STN CDR-H3 | KRSLSTPY |
| 853 | STN CDR-L1 | QSLLNRGNHKNY |
| 854 | STN CDR-L2 | WASTRES |
| 855 | STN CDR-L3 | QNDYTYPYT |
| 856 | STN VH | EVQLVQSGAEVKKPGASVKVSCKASGYTFTDHAIHWVRQAPGQG |
| | | LEWMGYFSPGNDDIKYNEKFRGRVTMTADKSSSTAYMELRSLRS |
| | | DDTAVYFCKRSLSTPYWGQGTLVTVSS |
| 857 | STN VL | DIVMTQSPDSLAVSLGERATINCKSSQSLLNRGNHKNYLTWYQQK |
| | | PGQPPKLLIYWASTRESGVPDRFSGSGSGTDFTLTISSLQAEDVAV |
| | | YYCQNDYTYPYTFGQGTKVEIK |
| 858 | CD33 CDR-H1 | NYDIN |
| 859 | CD33 CDR-H2 | WIYPGDGSTKYNEKFKA |
| 860 | CD33 CDR-H3 | GYEDAMDY |
| 861 | CD33 CDR-L1 | KASQDINSYLS |
| 862 | CD33 CDR-L2 | RANRLVD |
| 863 | CD33 CDR-L3 | LQYDEFPLT |
| 864 | CD33 VH | QVQLVQSGAE VKKPGASVKV SCKASGYTFT NYDINWVRQA |
| | | PGQGLEWIGW IYPGDGSTKY NEKFKAKATL TADTSTSTAY |
| | | MELRSLRSDD TAVYYCASGY EDAMDYWGQG TTVTVSS |
| 865 | CD33 VL | DIQMTQSPS SLSASVGDRVT |
| | | INCKASQDINSYLSWFQQKPGKAPKTL IYRANRLVDGVPS |
| | | RFSGSGSGQDYTLT ISSLQPEDFATYYCLQYDEFPLTFGGGTKVE |
| 866 | NTBA CDR-H1 | NYGMN |
| 867 | NTBA CDR-H2 | WINTYSGEPRYADDFKG |
| 868 | NTBA CDR-H3 | DYGRWYFDV |
| 869 | NTBA CDR-L1 | RASSSVSHMH |
| 870 | NTBA CDR-L2 | ATSNLAS |
| 871 | NTBA CDR-L3 | QQWSSTPRT |
| 872 | NTBA VH | QIQLVQSGSELKKPGASVKVSCKASGYTFTNYGMNWVRQAPGQD |
| | | LKWMGWINTYSGEPRYADDFKGRFVFSLDKSVNTAYLQISSLKAE |
| | | DTAVYYCARDYGRWYFDVWGQGTTVTVSS |
| 873 | NTBA VL | QIVLSQSPATLSLSPGERATMSCRASSSVSHMHWYQQKPGQAPRP |
| | | WIYATSNLASGVPARFSGSGSGTDYTLTISSLEPEDFAVYYCQQWS |
| | | STPRTFGGGTKVEIK |
| 874 | BCMA CDR-H1 | DYYIH |
| 875 | BCMA CDR-H2 | YINPNSGYTNYAQKFQG |
| 876 | BCMA CDR-H3 | YMWERVTGFFDF |
| 877 | BCMA CDR-L1 | LASEDISDDLA |
| 878 | BCMA CDR-L2 | TTSSLQS |
| 879 | BCMA CDR-L3 | QQTYKFPPT |
| 880 | BCMA VH | QVQLVQSGAEVKKPGASVKLSCKASGYTFTDYYIHWVRQAPGQG |
| | | LEWIGYINPNSGYTNYAQKFQGRATMTADKSINTAYVELSRLRSD |
| | | DTAVYFCTRYMWERVTGFFDFWGQGTMVTVSS |
| 881 | BCMA VL | DIQMTQSPSSVSASVGDRVTITCLASEDISDDLAWYQQKPGKAPK |
| | | VLVYTTSSLQSGVPSRFSGSGSGTDFTLTISSLQPEDFATYFCQQTY |
| | | KFPPTFGGGTKVEIK |
| 882 | TF CDR-H1 | GFTFSNYA |
| 883 | TF CDR-H2 | ISGSGDYT |
| 884 | TF CDR-H3 | ARSPWGYYLDS |
| 885 | TF CDR-L1 | QGISSR |
| 886 | TF CDR-L2 | AAS |
| 887 | TF CDR-L3 | QQYNSYPYT |
| 888 | TF VH | EVQLLESGGGLVQPGGSLRLSCAASGFTFSNYAMSWVRQAPGKG |
| | | LEWVSSISGSGDYTYYTDSVKGRFTISRDNSKNTLYLQMNSLRAE |
| | | DTAVYYCARSPWGYYLDSWGQGTLVTVSS |
| 889 | TF VL | DIQMTQSPPSLSASAGDRVTITCRASQGISSRLAWYQQKPEKAPKS |
| | | LIYAASSLQSGVPSRFSGSGSGTDFTLTISSLQPEDFATYYCQQYNS |
| | | YPYTFGQGTKLEIK |
| 890 | CD40 IgH | EVQLVESGGGLVQPGGSLRLSCAASGYSFTGYYIHWVRQAPGKG |
| | | LEWVARVIPNAGGTSYNQKFKGRFTLSVDNSKNTAYLQMNSLRA |
| | | EDTAVYYCAREGIYWWGQGTLVTVSSASTKGPSVFPLAPSSKSTS |
| | | GGTAALGCLVKDYFPEPVTVSWNSGALTSGVHTFPAVLQSSGLYS |
| | | LSSVVTVPSSSLGTQTYICNVNHKPSNTKVDKKVEPKSCDKTHTCP |
| | | PCPAPELLGGPSVFLFPPKPKDTLMISRTPEVTCVVVDVSHEDPEV |
| | | KFNWYVDGVEVHNAKTKPREEQYNSTYRVVSVLTVLHQDWLNG |
| | | KEYKCKVSNKALPAPIEKTISKAKGQPREPQVYTLPPSREEMTKNQ |
| | | VSLTCLVKGFYPSDIAVEWESNGQPENNYKTTPPVLDSDGSFFLYS |
| | | KLTVDKSRWQQGNVFSCSVMHEALHNHYTQKSLSLSPGK |
| 891 | CD40 IgL | DIQMTQSPSSLSASVGDRVTITCRSSQSLVHSNGNTFLHWYQQKP |
| | | GKAPKLLIYTVSNRFSGVPSRFSGSGSGTDFTLTISSLQPEDFATYF |
| | | CSQTTHVPWTFGQGTKVEIKRTVAAPSVFIFPPSDEQLKSGTASVV |
| | | CLLNNFYPREAKVQWKVDNALQSGNSQESVTEQDSKDSTYSLSST |
| | | LTLSKADYEKHKVYACEVTHQGLSSPVTKSFNRGEC |
| 892 | PD-1 IgH | QVQLVESGGGVVQPGRSLRLDCKASGITFSNSGMHWVRQAPGKG |
| | | LEWVAVIWYDGSKRYYADSVKGRFTISRDNSKNTLFLQMNSLRA |
| | | EDTAVYYCATNDDYWGQGTLVTVSSASTKGPSVFPLAPCSRSTSE |
| | | STAALGCLVKDYFPEPVTVSWNSGALTSGVHTFPAVLQSSGLYSL |
| | | SSVVTVPSSSLGTKTYTCNVDHKPSNTKVDKRVESKYGPPCPPCP |
| | | APEFLGGPSVFLFPPKPKDTLMISRTPEVTCVVVDVSQEDPEVQFN |
| | | WYVDGVEVHNAKTKPREEQFNSTYRVVSVLTVLHQDWLNGKEY |
| | | KCKVSNKGLPSSIEKTISKAKGQPREPQVYTLPPSQEEMTKNQVSL |
| | | TCLVKGFYPSDIAVEWESNGQPENNYKTTPPVLDSDGSFFLYSRLT |
| | | VDKSRWQEGNVFSCSVMHEALHNHYTQKSLSLSLGK |
| 893 | PD-1 IgL | EIVLTQSPATLSLSPGERATLSCRASQSVSSYLAWYQQKPGQAPRL |
| | | LIYDASNRATGIPARFSGSGSGTDFTLTISSLEPEDFAVYYCQQSSN |
| | | WPRTFGQGTKVEIKRTVAAPSVFIFPPSDEQLKSGTASVVCLLNNF |
| | | YPREAKVQWKVDNALQSGNSQESVTEQDSKDSTYSLSSTLTLSKA |
| | | DYEKHKVYACEVTHQGLSSPVTKSFNRGEC |
| 894 | PD-1 IgH | QVQLVQSGVEVKKPGASVKVSCKASGYTFTNYYMYWVRQAPGQ |
| | | GLEWMGGINPSNGGTNFNEKFKNRVTLTTDSSTTTAYMELKSLQF |
| | | DDTAVYYCARRDYRFDMGFDYWGQGTTVTVSSASTKGPSVFPLA |
| | | PCSRSTSESTAALGCLVKDYFPEPVTVSWNSGALTSGVHTFPAVL |
| | | QSSGLYSLSSVVTVPSSSLGTKTYTCNVDHKPSNTKVDKRVESKY |
| | | GPPCPPCPAPEFLGGPSVFLFPPKPKDTLMISRTPEVTCVVVDVSQE |
| | | DPEVQFNWYVDGVEVHNAKTKPREEQFNSTYRVVSVLTVLHQD |
| | | WLNGKEYKCKVSNKGLPSSIEKTISKAKGQPREPQVYTLPPSQEE |
| | | MTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK |
| 895 | PD-1 IgL | EIVLTQSPATLSLSPGERATLSCRASKGVSTSGYSYLHWYQQKPGQ |
| | | APRLLIYLASYLESGVPARFSGSGSGTDFTLTISSLEPEDFAVYYCQ |
| | | HSRDLPLTFGGGTKVEIKRTVAAPSVFIFPPSDEQLKSGTASVVCLL |
| | | NNFYPREAKVQWKVDNALQSGNSQESVTEQDSKDSTYSLSSTLTL |
| | | SKADYEKHKVYACEVTHQGLSSPVTKSFNRGEC |
| 896 | PD-L1 IgH | EVQLVESGGGLVQPGGSLRLSCAASGFTFSRYWMSWVRQAPGKG |
| | | LEWVANIKQDGSEKYYVDSVKGRFTISRDNAKNSLYLQMNSLRA |
| | | EDTAVYYCAREGGWFGELAFDYWGQGTLVTVSSASTKGPSVFPL |
| | | APSSKSTSGGTAALGCLVKDYFPEPVTVSWNSGALTSGVHTFPAV |
| | | LQSSGLYSLSSVVTVPSSSLGTQTYICNVNHKPSNTKVDKRVEPKS |
| | | CDKTHTCPPCPAPEFEGGPSVFLFPPKPKDTLMISRTPEVTCVVVD |
| | | VSHEDPEVKFNWYVDGVEVHNAKTKPREEQYNSTYRVVSVLTVL |
| | | HQDWLNGKEYKCKVSNKALPASIEKTISKAKGQPREPQVYTLPPS |
| | | REEMTKNQVSLTCLVKGFYPSDIAVEWESNGQPENNYKTTPPVLD |
| | | SDGSFFLYSKLTVDKSRWQQGNVFSCSVMHEALHNHYTQKSLSL |
| | | SPGK |
| 897 | PD-L1 IgL | EIVLTQSPGTLSLSPGERATLSCRASQRVSSSYLAWYQQKPGQAPR |
| | | LLIYDASSRATGIPDRFSGSGSGTDFTLTISRLEPEDFAVYYCQQYG |
| | | SLPWTFGQGTKVEIKRTVAAPSVFIFPPSDEQLKSGTASVVCLLNN |
| | | FYPREAKVQWKVDNALQSGNSQESVTEQDSKDSTYSLSSTLTLSK |
| | | ADYEKHKVYACEVTHQGLSSPVTKSFNRGEC |
| 898 | PD-L1 IgH | EVQLVESGGGLVQPGGSLRLSCAASGFTFSDSWIHWVRQAPGKGL |
| | | EWVAWISPYGGSTYYADSVKGRFTISADTSKNTAYLQMNSLRAE |
| | | DTAVYYCARRHWPGGFDYWGQGTLVTVSSASTKGPSVFPLAPSS |
| | | KSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDK |
| | | THTCPPCPAPELLGGPSVFLFPPKPKDTLMISRTPEVTCVVVDVSHE |
| | | DPEVKFNWYVDGVEVHNAKTKPREEQYASTYRVVSVLTVLHQD |
| | | WLNGKEYKCKVSNKALPAPIEKTISKAKGQPREPQVYTLPPSREE |
| | | MT |
| | | KNQVSLTCLVKGFYPSDIAVEWESNGQPENNYKTTPPVLDSDGSF |
| | | FLYSKLTVDKSRWQQGNVFSCSVMHEALHNHYTQKSLSLSPGK |
| 899 | PD-L1 IgL | DIQMTQSPSSLSASVGDRVTITCRASQDVSTAVAWYQQKPGKAPK |
| | | LLIYSASFLYSGVPSRFSGSGSGTDFTLTISSLQPEDFATYYCQQYL |
| | | YHPATFGQGTKVEIKRTVAAPSVFIFPPSDEQLKSGTASVVCLLNN |
| | | FYPREAKVQWKVDNALQSGNSQESVTEQDSKDSTYSLSSTLTLSK |
| | | ADYEKHKVYACEVTHQGLSSPVTKSFNRGEC |

In some embodiments, an antibody as described herein comprises a sequence which has at least 80% sequence identity to any one of SEQ ID NO: 1-899. In some embodiments, the antibody comprises a sequence which has at least 90% sequence identity to any one of SEQ ID NO: 1-899. In some embodiments, the antibody comprises a sequence which has at least 95% sequence identity to any one of SEQ ID NO: 1-899. In some embodiments, the antibody comprises a sequence which has at least 98% sequence identity to any one of SEQ ID NO: 1-899. In some embodiments, the antibody comprises a sequence which has at least 99% sequence identity to any one of SEQ ID NO: 1-899. In some embodiments, the antibody comprises a sequence which has at most 3 mutations relative to any one of SEQ ID NO: 1-899. In some embodiments, the antibody comprises a sequence which has at most 2 mutations relative to any one of SEQ ID NO: 1-899. In some embodiments, the antibody comprises a sequence which has at most 1 mutation relative to any one of SEQ ID NO: 1-899.

In some embodiments, an antibody as described herein targets CD40, BCMA, CD40, TIGIT, HER2, PD-1, PD-L1, or a combination thereof. In some embodiments, the antibody targets CD40, CD20, PD-1, PD-L1 or a combination thereof. In some embodiments, the antibody targets BCMA, TIGIT, HER2, or a combination thereof.

In some embodiments, an antibody as described herein (e.g., the antibody component of a MEF antibody) is a regulatory agency-approved, e.g., FDA- or EMA-approved therapeutic antibody. In some embodiments, the antibody described herein is selected from the group consisting of avelumab, durvalumab, daratumumab, elotuzumab, necitumumab, atezolizumab, nivolumab, dinutuximab, bevacizumab, pembrolizumab, ramucirumab, alemtuzumab, pertuzumab, obinutuzumab, ipilimumab, denosumab, ofatumumab, catumaxomab, panitumumab, bevacizumab, cetuximab, tositumomab, alemtuzumab, trastuzumab, rituximab, sintilimab, tislelizumab, camrelizumab, huJ591, JS001, hu3S193, TRC093, TRC105, AGEN1181, AGEN2034, MEDI4736, NEO-102, MK-0646, ZKAB001, TB-403, GLS-010, CT-011, INCMGA00012, AGEN1884, MK-3475, GC1118, DS-8201a, CC-95251, Sym004, CS1001, and REGN2810. In some embodiments, the antibody described herein is selected from the group consisting of rituximab, obinutuzumab, ofatumumab, trastuzumab, alemtuzumab, mogamulizumab, cetuximab, and dinutuximab. In some embodiments, the antibody described herein is rituximab. In some embodiments, the antibody described herein is obinutuzumab. In some embodiments, the antibody described herein is ofatumumab. In some embodiments, the antibody described herein is trastuzumab. In some embodiments, the antibody described herein is alemtuzumab. In some embodiments, the antibody described herein is mogamulizumab. In some embodiments, the antibody described herein is cetuximab. In some embodiments, the antibody described herein is dinutuximab.

### CD40-Targeting MEF Antibodies

In some embodiments, MEF antibodies of the present disclosure target Cluster of differentiation 40 (CD40). CD40 is a member of the tumor necrosis factor (TNF) receptor superfamily. It is a single chain type I transmembrane protein with an apparent MW of 50 kDa. Its mature polypeptide core consists of 237 amino acids, of which 173 amino acids comprise an extracellular domain (ECD) organized into 4 cysteine-rich repeats that are characteristic of TNF receptor family members. Two potential N-linked glycosylation sites are present in the membrane proximal region of the ECD, while potential O-linked glycosylation sites are absent. A 22 amino acid transmembrane domain connects the ECD with the 42 amino acid cytoplasmic tail of CD40. Sequence motifs involved in CD40-mediated signal transduction have been identified in the CD40 cytoplasmic tail. These motifs interact with cytoplasmic factors called TNF-R-associated factors (TRAFs) to trigger multiple downstream events including activation of MAP kinases and NFκB, which in turn modulate the transcriptional activities of a variety of inflammation-, survival-, and growth-related genes. See, e.g., van Kooten and Banchereau, J. Leukoc. Biol. 67:2-17 (2000); Elgueta et al., Immunol. Rev. 229:152-172 (2009).

Within the hematopoietic system, CD40 can be found on B cells at multiple stages of differentiation, monocytes, macrophages, platelets, follicular dendritic cells, dendritic cells (DC), eosinophils, and activated T cells. In normal non-hematopoietic tissues, CD40 has been detected on renal epithelial cells, keratinocytes, fibroblasts of synovial membrane and dermal origins, and activated endothelium. A soluble version of CD40 is released from CD40-expressing cells, possibly through differential splicing of the primary transcript or limited proteolysis by the metalloproteinase TNFα converting enzyme. Shed CD40 can potentially modify immune responses by interfering with the CD40/CD40L interaction. See, e.g., van Kooten and Banchereau, J. Leukoc. Biol. 67:2-17 (2000); Elgueta et al., Immunol. Rev. 229:152-172 (2009).

The endogenous ligand for CD40 (CD40L) is a type II membrane glycoprotein of 39 kDa also known as CD154. CD40L is a member of the TNF superfamily and is expressed as a trimer on the cell surface. CD40L is transiently expressed on activated CD4+, CD8+, and γδ T cells. CD40L is also detected at variable levels on purified monocytes, activated B cells, epithelial and vascular endothelial cells, smooth muscle cells, and DCs, but the functional relevance of CD40L expression on these cell types has not been clearly defined (van Kooten 2000; Elgueta 2009). However, expression of CD40L on activated platelets has been implicated in the pathogenesis of thrombotic diseases. See, e.g., Ferroni et al., Curr. Med. Chem. 14:2170-2180 (2007).

The best-characterized function of the CD40/CD40L interaction is its role in contact-dependent reciprocal interaction between antigen-presenting cells and T cells. See, e.g., van Kooten and Banchereau, J. Leukoc. Biol. 67:2-17 (2000); Elgueta et al., Immunol. Rev. 229:152-172 (2009). Binding of CD40L on activated T cells to CD40 on antigen-activated B cells not only drives rapid B cell expansion, but also provides an essential signal for B cells to differentiate into either memory B cells or plasma cells. CD40 signaling is responsible for the formation of germinal centers in which B cells undergo affinity maturation and isotype switching to acquire the ability to produce high affinity antibodies of the IgG, IgA, and IgE isotypes. See, e.g., Kehry, J. Immunol. 156:2345-2348 (1996). Thus, individuals with mutations in the CD40L locus that prevent functional CD40/CD40L interaction suffer from the primary immunodeficiency X-linked hyper-IgM syndrome that is characterized by over-representation of circulating IgM and the inability to produce IgG, IgA, and IgE. These patients demonstrate suppressed secondary humoral immune responses, increased susceptibility to recurrent pyrogenic infections, and a higher frequency of carcinomas and lymphomas. Gene knockout experiments in mice to inactivate either CD40 or CD40L locus reproduce the major defects seen in X-linked hyper-IgM patients. These KO mice also show impaired antigen-specific T cell priming, suggesting that the CD40L/CD40 interaction is also a critical factor for mounting cell-mediated immune responses. See, e.g., Elgueta et al., Immunol. Rev. 229:152-172 (2009).

The immune-stimulatory effects of CD40 ligation by CD40L or anti-CD40 in vivo have correlated with immune responses against syngeneic tumors. See, e.g., French et al., Nat. Med. 5:548-553 (1999). A deficient immune response against tumor cells can result from a combination of factors such as expression of immune checkpoint molecules, such as PD1 or CTLA-4, decreased expression of MI-IC antigens, poor expression of tumor-associated antigens, appropriate adhesion, or co-stimulatory molecules, and the production of immunosuppressive proteins like TGFβ by the tumor cells. CD40 ligation on antigen presenting and transformed cells results in up-regulation of adhesion proteins (e.g., CD54), co-stimulatory molecules (e.g., CD86) and WIC antigens, as well as inflammatory cytokine secretion, thereby potentially inducing and/or enhancing the antitumor immune response, as well as the immunogenicity of the tumor cells. See, e.g., Gajewski et al., Nat. Immunol. 14:1014-1022 (2013).

A primary consequence of CD40 cross-linking is DC activation (often termed licensing) and potentiation of myeloid and B cells ability to process and present tumor-associated antigens to T cells. Besides having a direct ability to activate the innate immune response, a unique consequence of CD40 signaling is APC presentation of tumor-derived antigens to CD8+ cytotoxic T cell (CTL) precursors in a process known as ‘cross-priming’. This CD40-dependent activation and differentiation of CTL precursors by mature DCs into tumor-specific effector CTLs can enhance cell-mediated immune responses against tumor cells. See, e.g., Kurts et al., Nat. Rev. Immunol. 10:403-414 (2010).

In certain aspects, the present disclosure provides a MEF anti-CD40 antibody. Amino acid sequences of a heavy chain and a light chain for a humanized anti-CD40 antibody which may be an MEF antibody of the present disclosure are disclosed as SEQ ID NO: 890 and 891, respectively, wherein the variable region of the heavy chain is from amino acids 1-113 of SEQ ID NO: 890 and the variable region of the light chain is from amino acids 1-113 of SEQ ID NO: 891.

In some embodiments, the anti-CD40 antibody comprises a sequence which has at least 80% sequence identity to SEQ ID NO: 890. In some embodiments, the anti-CD40 antibody comprises a sequence which has at least 90% sequence identity to SEQ ID NO: 890. In some embodiments, the anti-CD40 antibody comprises a sequence which has at least 95% sequence identity to SEQ ID NO: 890. In some embodiments, the anti-CD40 antibody comprises a sequence which has at least 98% sequence identity to SEQ ID NO: 890. In some embodiments, the anti-CD40 antibody comprises a sequence which has at least 99% sequence identity to SEQ ID NO: 890. In some embodiments, the anti-CD40 antibody comprises a sequence which has at least 80% sequence identity to SEQ ID NO: 891. In some embodiments, the anti-CD40 antibody comprises a sequence which has at least 90% sequence identity to SEQ ID NO: 891. In some embodiments, the anti-CD40 antibody comprises a sequence which has at least 95% sequence identity to SEQ ID NO: 891. In some embodiments, the anti-CD40 antibody comprises a sequence which has at least 98% sequence identity to SEQ ID NO: 891. In some embodiments, the anti-CD40 antibody comprises a sequence which has at least 99% sequence identity to SEQ ID NO: 891.

In some embodiments, the anti-CD40 antibody comprises a first sequence which has at least 80% sequence identity to SEQ ID NO: 890 and a second sequence which has at least 80% sequence identity to SEQ ID NO: 891. In some embodiments, the anti-CD40 antibody comprises a first sequence which has at least 90% sequence identity to SEQ ID NO: 890 and a second sequence which has at least 90% sequence identity to SEQ ID NO: 891. In some embodiments, the anti-CD40 antibody comprises a first sequence which has at least 95% sequence identity to SEQ ID NO: 890 and a second sequence which has at least 95% sequence identity to SEQ ID NO: 891. In some embodiments, the anti-CD40 antibody comprises a first sequence which has at least 98% sequence identity to SEQ ID NO: 890 and a second sequence which has at least 98% sequence identity to SEQ ID NO: 891. In some embodiments, the anti-CD40 antibody comprises a first sequence which has at least 99% sequence identity to SEQ ID NO: 890 and a second sequence which has at least 99% sequence identity to SEQ ID NO: 891.

In some cases, the anti-CD40 antibody has a dissociation constant (K_{D}) for human CD40 of at most 500 nM. In some cases, the anti-CD40 antibody has a dissociation constant (K_{D}) for human CD40 of at most 100 nM. In some cases, the anti-CD40 antibody has a dissociation constant (K_{D}) for human CD40 of at most 50 nM. In some cases, the anti-CD40 antibody has a dissociation constant (K_{D}) for human CD40 of at most 10 nM. In some cases, the anti-CD40 antibody has a dissociation constant (K_{D}) for human CD40 of at most 5 nM. In some cases, the anti-CD40 antibody has a dissociation constant (K_{D}) for human CD40 of at most 1 nM. In some cases, the anti-CD40 antibody has a dissociation constant (K_{D}) for human CD40 of at most 500 pM.

In many instances, the MEF anti-CD40 antibody comprises one or more BPM functionalizations. For example, each interchain disulfide can be reduced and reversibly coupled to effector function-diminishing PEG moieties. Furthermore, in many instances, the MEF anti-CD40 antibody comprises an effector function enhancing modification, such as afucosylation. Modulated effector function of the MEF anti-CD40 antibody can result in lower toxicity, diminished b cell depletion, enhanced activity localization, and improved half-life relative to antibodies lacking the effector function modulations.

Anti-CD40 MEF antibodies of the present disclosure (as well as fragments, chimeric constructs, fusion constructs, and mutants thereof) can exhibit enhanced binding to FcγIII receptors, as well as enhanced ability to activate the CD40 signaling pathway in immune cells. In many cases, these antibodies act as agonists or partial agonists of the CD40 signaling pathway. In many cases, these antibodies bind to human CD40 protein, and can activate the CD40 signaling pathway.

In some embodiment, a humanized anti-CD40 antibody disclosed herein is useful in the treatment of various disorders associated with the expression of CD40 as described herein. Because these antibodies can activate the immune system to respond against tumor-related antigens, their uses are not limited to cancers that express CD40. Thus, these antibodies can be used to treat both CD40 positive and CD40 negative cancers.

### PD-1 and PD-L1-Targeting Antibodies

In certain embodiments, the antibody that binds an immune cell engager is a PD-1/PD-L1 inhibitor. Examples of PD-1/PD-L1 inhibitors include, but are not limited to, those described in U.S. Pat. Nos. 7,488,802; 7,943,743; 8,008,449; 8,168,757; 8,217,149, and PCT Patent Application Publication Nos. WO2003042402, WO2008156712, WO2010089411, WO2010036959, WO2011066342, WO2011159877, WO2011082400, and WO2011161699, all of which are incorporated herein in their entireties.

In some embodiments, the antibody that binds an immune cell engager is a PD-1 inhibitor. In one embodiment, the PD-1 inhibitor is an anti-PD-1 antibody. In one embodiment, the anti-PD-1 antibody is BGB-A317, nivolumab (also known as ONO-4538, BMS-936558, or MDX1106), pembrolizumab (also known as MK-3475, SCH 900475, or lambrolizumab), or a nonfucosylated version thereof. In one embodiment, the anti-PD-1 antibody is nivolumab or a nonfucosylated version thereof. Nivolumab is a human IgG4 anti-PD-1 monoclonal antibody, and is marketed under the trade name Opdivo™. In another embodiment, the anti-PD-1 antibody is pembrolizumab or a nonfucosylated version thereof. Pembrolizumab is a humanized monoclonal IgG4 antibody and is marketed under the trade name Keytruda™. In yet another embodiment, the anti-PD-1 antibody is CT-011, a humanized antibody, or a nonfucosylated version thereof. CT-011 administered alone has failed to show response in treating acute myeloid leukemia (AML) at relapse. In yet another embodiment, the anti-PD-1 antibody is AMP-224, a fusion protein, or a nonfucosylated version thereof. In another embodiment, the PD-1 antibody is BGB-A317, or a nonfucosylated version thereof. BGB-A317 is a monoclonal antibody in which the ability to bind Fc gamma receptor I is specifically engineered out, and which has a unique binding signature to PD-1 with high affinity and superior target specificity. In one embodiment, the PD-1 antibody is cemiplimab or a nonfucosylated version thereof. In another embodiment, the PD-1 antibody is camrelizumab or a nonfucosylated version thereof. In a further embodiment, the PD-1 antibody is sintilimab or a nonfucosylated version thereof. In some embodiments, the PD-1 antibody is tislelizumab or a nonfucosylated version thereof. In certain embodiments, the PD-1 antibody is TSR-042 or a nonfucosylated version thereof. In yet another embodiment, the PD-1 antibody is PDR001 or a nonfucosylated version thereof. In yet another embodiment, the PD-1 antibody is toripalimab or a nonfucosylated version thereof.

In certain aspects, the present disclosure provides a MEF anti-PD-1 antibody. In some cases, amino acid sequences of a heavy chain and a light chain for a humanized anti-PD-1 antibody are SEQ ID NO: 892 and 893, respectively. In some cases, amino acid sequences of a heavy chain and a light chain for a humanized anti-PD-1 antibody are SEQ ID NO: 894 and 895, respectively.

In some embodiments, the anti-PD-1 antibody comprises a sequence which has at least 80% sequence identity to SEQ ID NO: 892. In some embodiments, the anti-PD-1 antibody comprises a sequence which has at least 90% sequence identity to SEQ ID NO: 892. In some embodiments, the anti-PD-1 antibody comprises a sequence which has at least 95% sequence identity to SEQ ID NO: 892. In some embodiments, the anti-PD-1 antibody comprises a sequence which has at least 98% sequence identity to SEQ ID NO: 892. In some embodiments, the anti-PD-1 antibody comprises a sequence which has at least 99% sequence identity to SEQ ID NO: 892. In some embodiments, the anti-PD-1 antibody comprises a sequence which has at least 80% sequence identity to SEQ ID NO: 893. In some embodiments, the anti-PD-1 antibody comprises a sequence which has at least 90% sequence identity to SEQ ID NO: 893. In some embodiments, the anti-PD-1 antibody comprises a sequence which has at least 95% sequence identity to SEQ ID NO: 893. In some embodiments, the anti-PD-1 antibody comprises a sequence which has at least 98% sequence identity to SEQ ID NO: 893. In some embodiments, the anti-PD-1 antibody comprises a sequence which has at least 99% sequence identity to SEQ ID NO: 893. In some embodiments, the anti-PD-1 antibody comprises a sequence which has at least 80% sequence identity to SEQ ID NO: 894. In some embodiments, the anti-PD-1 antibody comprises a sequence which has at least 90% sequence identity to SEQ ID NO: 894. In some embodiments, the anti-PD-1 antibody comprises a sequence which has at least 95% sequence identity to SEQ ID NO: 894. In some embodiments, the anti-PD-1 antibody comprises a sequence which has at least 98% sequence identity to SEQ ID NO: 894. In some embodiments, the anti-PD-1 antibody comprises a sequence which has at least 99% sequence identity to SEQ ID NO: 894. In some embodiments, the anti-PD-1 antibody comprises a sequence which has at least 80% sequence identity to SEQ ID NO: 895. In some embodiments, the anti-PD-1 antibody comprises a sequence which has at least 90% sequence identity to SEQ ID NO: 895. In some embodiments, the anti-PD-1 antibody comprises a sequence which has at least 95% sequence identity to SEQ ID NO: 895. In some embodiments, the anti-PD-1 antibody comprises a sequence which has at least 98% sequence identity to SEQ ID NO: 895. In some embodiments, the anti-PD-1 antibody comprises a sequence which has at least 99% sequence identity to SEQ ID NO: 895.

In certain embodiments, the antibody that binds an immune cell engager is a PD-L1 inhibitor. In one embodiment, the PD-L1 inhibitor is an anti-PD-L1 antibody. In one embodiment, the anti-PD-L1 antibody is MEDI4736 (durvalumab) or a nonfucosylated version thereof. In another embodiment, the anti-PD-L1 antibody is BMS-936559 (also known as MDX-1105-01) or a nonfucosylated version thereof. In yet another embodiment, the PD-L1 inhibitor is atezolizumab (also known as MPDL3280A, and Tecentriq®) or a nonfucosylated version thereof. In a further embodiment, the PD-L1 inhibitor is avelumab or a nonfucosylated version thereof.

In certain aspects, the present disclosure provides a MEF anti-PD-L1 antibody. In some cases, amino acid sequences of a heavy chain and a light chain for a humanized anti-PD-L1 antibody are SEQ ID NO: 896 and 897, respectively. In some cases, amino acid sequences of a heavy chain and a light chain for a humanized anti-PD-L1 antibody are SEQ ID NO: 898 and 899, respectively.

In some embodiments, the anti-PD-L1 antibody comprises a sequence which has at least 80% sequence identity to SEQ ID NO: 896. In some embodiments, the anti-PD-L1 antibody comprises a sequence which has at least 90% sequence identity to SEQ ID NO: 896. In some embodiments, the anti-PD-L1 antibody comprises a sequence which has at least 95% sequence identity to SEQ ID NO: 896. In some embodiments, the anti-PD-L1 antibody comprises a sequence which has at least 98% sequence identity to SEQ ID NO: 896. In some embodiments, the anti-PD-L1 antibody comprises a sequence which has at least 99% sequence identity to SEQ ID NO: 896. In some embodiments, the anti-PD-L1 antibody comprises a sequence which has at least 80% sequence identity to SEQ ID NO: 897. In some embodiments, the anti-PD-L1 antibody comprises a sequence which has at least 90% sequence identity to SEQ ID NO: 897. In some embodiments, the anti-PD-L1 antibody comprises a sequence which has at least 95% sequence identity to SEQ ID NO: 897. In some embodiments, the anti-PD-L1 antibody comprises a sequence which has at least 98% sequence identity to SEQ ID NO: 897. In some embodiments, the anti-PD-L1 antibody comprises a sequence which has at least 99% sequence identity to SEQ ID NO: 897. In some embodiments, the anti-PD-L1 antibody comprises a sequence which has at least 80% sequence identity to SEQ ID NO: 898. In some embodiments, the anti-PD-L1 antibody comprises a sequence which has at least 90% sequence identity to SEQ ID NO: 898. In some embodiments, the anti-PD-L1 antibody comprises a sequence which has at least 95% sequence identity to SEQ ID NO: 898. In some embodiments, the anti-PD-L1 antibody comprises a sequence which has at least 98% sequence identity to SEQ ID NO: 898. In some embodiments, the anti-PD-L1 antibody comprises a sequence which has at least 99% sequence identity to SEQ ID NO: 898. In some embodiments, the anti-PD-L1 antibody comprises a sequence which has at least 80% sequence identity to SEQ ID NO: 899. In some embodiments, the anti-PD-L1 antibody comprises a sequence which has at least 90% sequence identity to SEQ ID NO: 899. In some embodiments, the anti-PD-L1 antibody comprises a sequence which has at least 95% sequence identity to SEQ ID NO: 899. In some embodiments, the anti-PD-L1 antibody comprises a sequence which has at least 98% sequence identity to SEQ ID NO: 899. In some embodiments, the anti-PD-L1 antibody comprises a sequence which has at least 99% sequence identity to SEQ ID NO: 899.

### MEF Antibody Activity

In some embodiments, when a MEF antibody as described herein is introduced to a population of cells comprising one or more target cells, the binding of the MEF antibody to the one or more target cells provides a time-dependent reduction in the peripheral cytokine levels relative to peripheral cytokine levels provided by binding of an equimolar amount of the equivalent antibody lacking the BPM. In some embodiments, the peripheral cytokine levels are reduced for a period of time.

Peripheral cytokine levels in a subject can refer to systemic or circulating cytokine levels. For example, in a subject with a solid tumor, central or local cytokine levels occur in the region substantially around the solid tumor, while peripheral cytokine levels could be measured, for example, in a blood or plasma sample. In some embodiments, the peripheral cytokines described herein are selected from the group consisting of EGF, Eotaxin, G-CSF, GM-CSF, IFNα2, IFNγ, IL-10, IL-12P40, IL-12P70, IL-13, IL-15, IL-17A, IL-1RA, IL-1α, IL-1β, IL-2, IL-3, IL-4, IL-5, IL-6, IL-7, IL-8, IP-10, MCP-1, MIP-1α, MIP-1β, RANTES, TNFα, TNFβ, VEGF, FGF-2, TGF-α, FIT-3L, Fractalkine, GRO, MCP-3, MDC, PDGF-AA, PDGF-AB/BB, sCD40L, IL-9, and combinations of any of the foregoing.

In some embodiments, the peripheral cytokine levels are reduced by about 1% to about 80%. For example, about 1% to about 20%, about 10% to about 30%, about 20% to about 40%, about 30% to about 50%, about 40% to about 60%, about 50% to about 70%, about 60% to about 80%, or any value in between. In some embodiments, the period of time is from about 4 hours to about 24 hours after the MEF antibody described herein is introduced to the population of cells, and the peripheral cytokine levels are reduced by about 1% to about 20%. In some embodiments, the period of time is from about 4 hours to about 48 hours after the MEF antibody described herein is introduced to the population of cells, and the peripheral cytokine levels are reduced by about 1% to about 20%. In some embodiments, the period of time is from about 24 hours to about 48 hours after the MEF antibody described herein is introduced to the population of cells, and the peripheral cytokine levels are reduced by about 1% to about 20%. In some embodiments, the period of time is from about 36 hours to about 72 hours after the MEF antibody described herein is introduced to the population of cells, and the peripheral cytokine levels are reduced by about 1% to about 20%. In some embodiments, the period of time is from about 48 hours to about 96 hours after the MEF antibody described herein is introduced to the population of cells, and the peripheral cytokine levels are reduced by about 1% to about 20%.

In some embodiments, the time-dependent reduction in peripheral cytokine levels is characterized by an initial reduction in peripheral cytokine levels in the supernatant of the biological sample relative to that from an equimolar amount of the equivalent antibody. In some embodiments, the time-dependent reduction of peripheral cytokine levels is characterized by an initial reduction of at least about 50%. In some embodiments, the time-dependent reduction of peripheral cytokine levels is characterized by an initial reduction of at least about 80%.

In some embodiments, the initial reduction comprises a period of time from the administration of the MEF antibody to a subject (e.g., “0 hours” post-administration) and about 3 hours after administration of the MEF antibody to the subject. For example, about 0 hours to about 2 hours post-administration, about 0 hours to about 1.5 hours post-administration, about 0 hours to about 1 hour post-administration, about 0 hours to about 0.5 hours post-administration, about 0.5 hours to about 2 hours post administration, or about 0.5 hours to 1.5 hours post-administration.

In some embodiments, the time-dependent reduction of peripheral cytokine levels is characterized by recovery of the peripheral cytokine levels to at least about 50% relative to that from an equimolar amount of the equivalent antibody after from about 48 h to about 96 h. In some embodiments, the time-dependent reduction of peripheral cytokine levels is characterized by recovery of the peripheral cytokine levels to about 100% relative to that from an equimolar amount of the equivalent antibody after from about 48 h to about 96 h.

In some embodiments, when a MEF antibody as described herein is introduced to a population of cells comprising one or more target cells, the binding of the MEF antibody to the one or more target cells provides a time-dependent reduction in the rate of cell lysis of the one or more target cells relative to the rate of cell lysis provided by binding of an equimolar amount of an equivalent antibody.

In some embodiments, the population of cells is a biological sample. In some embodiments, the population of cells is in a subject.

In some embodiments, the population of cells is in a subject; and the peripheral cytokine levels are systemic cytokine levels in the plasma of the subject.

In some embodiments, administration of an antibody as described herein to a subject provides a reduction of about 20% to about 75% in cytokine Cₘₐₓ relative to administration of an equimolar amount of an equivalent antibody. In some embodiments, the reduction in cytokine Cₘₐₓ is about 20% to about 40%, about 30% to about 50%, about 40% to about 60%, about 50% to about 75%, or any value in between.

In some embodiments, administration of an antibody as described herein to a subject provides substantially the same total antibody AUC_{0-∞} relative to administration of an equimolar amount of an equivalent antibody.

In some embodiments, administration to a subject of a MEF antibody provides a reduction in effector function relative to administration to the subject of an equivalent antibody. In some embodiments, the effector function that is reduced relative to an equivalent antibody on administration of the MEF antibody to a subject is antibody-dependent cellular cytotoxicity (ADCC) and antibody-dependent cellular phagocytosis (ADCP). In some embodiments, the effector function that is reduced relative to an equivalent antibody on administration of the MEF antibody to a subject is antibody-dependent cellular cytotoxicity (ADCC) or antibody-dependent cellular phagocytosis (ADCP). In some embodiments, the effector function that is reduced relative to an equivalent antibody on administration of the MEF antibody to a subject is antibody-dependent cellular cytotoxicity (ADCC). In some embodiments, the effector function that is reduced relative to an equivalent antibody on administration of the MEF antibody to a subject is antibody-dependent cellular phagocytosis (ADCP).

### Methods of Making Afucosylated Antibodies

Methods of making afucosylated antibodies by incubating antibody-producing cells with a fucose analogue are described, e.g., in WO2009/135181. Briefly, cells that have been engineered to express antibodies of the present disclosure are incubated in the presence of a fucose analogue or an intracellular metabolite or product of the fucose analog. An intracellular metabolite can be, for example, a GDP-modified analog or a fully or partially de-esterified analog. A product can be, for example, a fully or partially de-esterified analog. In some embodiments, a fucose analogue can inhibit an enzyme(s) in the fucose salvage pathway. For example, a fucose analog (or an intracellular metabolite or product of the fucose analog) can inhibit the activity of fucokinase, or GDP-fucose-pyrophosphorylase. In some embodiments, a fucose analog (or an intracellular metabolite or product of the fucose analog) inhibits fucosyltransferase (preferably a 1,6-fucosyltransferase, e.g., the FUT8 protein). In some embodiments, a fucose analog (or an intracellular metabolite or product of the fucose analog) can inhibit the activity of an enzyme in the de novo synthetic pathway for fucose. For example, a fucose analog (or an intracellular metabolite or product of the fucose analog) can inhibit the activity of GDP-mannose 4,6-dehydratase or/or GDP-fucose synthetase. In some embodiments, the fucose analog (or an intracellular metabolite or product of the fucose analog) can inhibit a fucose transporter (e.g., GDP-fucose transporter).

In one embodiment, the fucose analogue is 2-flurofucose. Methods of using fucose analogues in growth medium and other fucose analogues are disclosed, e.g., in WO 2009/135181, which is herein incorporated by reference.

Other methods for engineering cell lines to reduce core fucosylation included gene knock-outs, gene knock-ins and RNA interference (RNAi). In gene knock-outs, the gene encoding FUT8 (alpha 1,6-fucosyltransferase enzyme) is inactivated. FUT8 catalyzes the transfer of a fucosyl residue from GDP-fucose to position 6 of Asn-linked (N-linked) GlcNac of an N-glycan. FUT8 is reported to be the only enzyme responsible for adding fucose to the N-linked biantennary carbohydrate at Asn297. Gene knock-ins add genes encoding enzymes such as GNTIII or a golgi alpha mannosidase II. An increase in the levels of such enzymes in cells diverts monoclonal antibodies from the fucosylation pathway (leading to decreased core fucosylation), and having increased amount of bisecting N-acetylglucosamines. RNAi typically also targets FUT8 gene expression, leading to decreased mRNA transcript levels or knocking out gene expression entirely. Any of these methods can be used to generate a cell line that would be able to produce an afucosylated antibody.

Many methods are available to determine the amount of fucosylation on an antibody. Methods include, e.g., LC-MS via PLRP-S chromatography, electrospray ionization quadrupole TOF MS, Capillary Electrophoresis with Laser-Induced Fluorescence (CE-LIF) and, Hydrophilic Interaction Chromatography with Fluorescence Detection (HILIC).

### Assays

Some embodiments provide assays for assessing antibody-dependent cellular cytotoxicity (ADCC) and/or antibody-dependent cellular phagocytosis (ADCP). In some embodiments, when a MEF antibody as described herein is introduced to a population of cells comprising one or more target cells, the peripheral cytokine levels are reduced for a period of time relative to an equimolar amount of an equivalent antibody. Peripheral cytokine levels refers to cytokine levels in regions where there are no target cells. For example, in cell culture, peripheral cytokine levels can refer to cytokine levels in the cell culture media or supernatant. In some embodiments, the population of cells is a biological sample; and the peripheral cytokine levels are reduced in the supernatant. In some embodiments, the population of cells is in a subject; and the peripheral cytokine levels are systemic cytokine levels in the plasma of the subject. In some embodiments, the period of time is from about 12 hours to about 36 hours after the MEF antibody is introduced to the population of cells, and the peripheral cytokine levels are reduced by about 1% to about 20%. In some embodiments, the period of time is from about 16 hours to about 24 hours after the MEF antibody is introduced to the population of cells, and the peripheral cytokine levels are reduced by about 1% to about 20%.

In some embodiments, the population of cells is a biological sample. In some embodiments, the one or more target cells comprise cancer cells comprising antigens, or immune cells comprising antigens. In some embodiments, the population of cells further comprises normal peripheral blood mononuclear cells (PBMCs). In some embodiments, the normal PBMCs comprise natural killer cells.

In some embodiments, the target cells further comprise a radiolabel (i.e., the cells are radiolabeled). In some embodiments, the radiolabel is released into the cell culture media or supernatant upon cell lysis.

In some embodiments, the Fc receptor in an antibody assay is present on a PBMC. In some embodiments, the Fc receptor is the Fc gamma receptor III. In some embodiments, the PBMC is a natural killer cell. In some embodiments, the PBMC is enriched from plasma of a normal donor. In some embodiments, the normal donor is a human having the Fc gamma receptor III 158 V/V genotype. In some embodiments, reduction in Fc receptor binding is determined by competitive binding of the MEF antibody and a labeled isotype matched IgG Fc fragment to an orthogonally labeled Fc receptor.

In some embodiments, the IgG Fc fragment is the labeled isotype matched Fc domain of a human IgG₁ antibody. In some embodiments, the label of the isotype matched IgG Fc fragment comprises a fluorophore. Exemplary fluorophores include, but are not limited to coumarins, Alexa fluors, cyanines, rhodamines, and BODIPY. In some embodiments, the labeled isotype matched IgG Fc fragment is immobilized on a solid support. In some embodiments, the orthogonal label of the Fc receptor comprises biotin. In some embodiments, the Fc receptor is Fc gamma Ma or Fc gamma Mb. In some embodiments, the Fc receptor is Fc gamma Ma. In some embodiments, the Fc receptor is Fc gamma IIIb.

In some embodiments, when an antibody as described herein is introduced to a population of cells comprising one or more target cells, the lysis of the one or more target cells is reduced for a period of time relative to an equimolar amount of an equivalent antibody. In some embodiments, the lysis of the one or more target cells is reduced by about 1% to about 80%. For example, about 1% to about 20%, about 10% to about 30%, about 20% to about 40%, about 30% to about 50%, about 40% to about 60%, about 50% to about 70%, about 60% to about 80%, or any value in between. In some embodiments, the period of time is from about 48 hours to about 96 hours after the MEF antibody is introduced to the population of cells, and the lysis of the one or more target cells is reduced by about 1% to about 20%.

### Compositions and Methods of Administration

The present disclosure provides pharmaceutical compositions comprising the MEF antibodies described herein. Some embodiments provide a pharmaceutical composition comprising an MEF antibody and a pharmaceutically acceptable carrier. In some embodiments, the composition comprises a distribution of MEF antibodies. In some embodiments, the sole active ingredient in the composition is the MEF antibody.

While therapeutic antibodies often affect high levels of systemic cytokine release upon administration, many of the modulated effector antibodies disclosed herein exhibit diminished and/or delayed effector function activities, thereby lowering the risk for cytokine release syndrome. During cases of cytokine release syndrome, wherein the cytokine or the inflammatory marker is monocyte chemotactic protein-1 (MCP-1), macrophage inflammatory protein-1 (MIP-1β), tumor necrosis factor (TNF-α), interferon gamma (IFN-γ), interleukin-1 receptor agonist (IL-1RA), interleukin 1 beta (IL1B), interleukin 6 (IL6), interleukin 10 (IL10), or a combination thereof often undergo multi-fold serum-level increases which can affect systemic toxicities, allowing these species to serve as useful markers for antibody toxicities.

In some cases, a unit dose of the composition does not increase systemic levels of monocyte chemotactic protein-1 (MCP-1), macrophage inflammatory protein-1 (MIP-1β), tumor necrosis factor (TNF-α), interferon gamma (IFN-γ), interleukin-1 receptor agonist (IL-1RA), interleukin 1 beta (IL1B), interleukin 6 (IL6), interleukin 10 (IL10), or a combination thereof by more than 20-fold above levels prior to the administering (e.g., peak level as measured by an ELISA assay on plasma collected from a subject). In some cases, a unit dose of the composition does not increase systemic levels of monocyte chemotactic protein-1 (MCP-1), macrophage inflammatory protein-1 (MIP-1β), tumor necrosis factor (TNF-α), interferon gamma (IFN-γ), interleukin-1 receptor agonist (IL-1RA), interleukin 1 beta (IL1B), interleukin 6 (IL6), interleukin 10 (IL10), or a combination thereof by more than 10-fold above levels prior to the administering (e.g., as measured by an ELISA assay on plasma collected from a subject). In some cases, a unit dose of the composition does not increase systemic levels of monocyte chemotactic protein-1 (MCP-1), macrophage inflammatory protein-1 (MIP-1β), tumor necrosis factor (TNF-α), interferon gamma (IFN-γ), interleukin-1 receptor agonist (IL-1RA), interleukin 1 beta (IL1B), interleukin 6 (IL6), interleukin 10 (IL10), or a combination thereof by more than 5-fold above levels prior to the administering (e.g., as measured by an ELISA assay on plasma collected from a subject). In some cases, a unit dose of the composition does not increase systemic levels of monocyte chemotactic protein-1 (MCP-1), macrophage inflammatory protein-1 (MIP-1β), tumor necrosis factor (TNF-α), interferon gamma (IFN-γ), interleukin-1 receptor agonist (IL-1RA), interleukin 1 beta (IL1B), interleukin 6 (IL6), interleukin 10 (IL10), or a combination thereof by more than 3-fold above levels prior to the administering (e.g., as measured by an ELISA assay on plasma collected from a subject). In some cases, a unit dose of the composition does not increase systemic levels of MCP-1 by more than 100 pg/mL, by more than 400 pg/mL, or by more than 800 pg/mL. In some cases, a unit dose of the composition does not increase systemic levels of TNF-α by more than 15 pg/mL, by more than 60 pg/mL, or by more than 120 pg/mL. In some cases, a unit dose of the composition does not increase systemic levels of IFN-γ by more than 25 pg/mL, by more than 100 pg/mL, or by more than 200 pg/mL. In some cases, a unit dose of the composition does not increase systemic levels of IL1B by more than 2 pg/mL, by more than 8 pg/mL, or by more than 20 pg/mL. In some cases, a unit dose of the composition does not increase systemic levels of IL6 by more than 1 pg/mL, by more than 4 pg/mL, or by more than 10 pg/mL. In some cases, a unit dose of the composition does not increase systemic levels of IL6 by more than 10 pg/mL, by more than 40 pg/mL, or by more than 100 pg/mL.

In some cases, a unit dose of the composition does not increase systemic levels of monocyte chemotactic protein-1 (MCP-1) by more than 20-fold above levels prior to the administering (e.g., as measured by an ELISA assay on plasma collected from a subject). In some cases, a unit dose of the composition does not increase systemic levels of monocyte chemotactic protein-1 (MCP-1) by more than 10-fold above levels prior to the administering. In some cases, a unit dose of the composition does not increase systemic levels of monocyte chemotactic protein-1 (MCP-1) by more than 5-fold above levels prior to the administering. In some cases, a unit dose of the composition does not increase systemic levels of monocyte chemotactic protein-1 (MCP-1) by more than 3-fold above levels prior to the administering. In some cases, a unit dose of the composition does not increase systemic levels of MCP-1 by more than 100 pg/mL, by more than 400 pg/mL, or by more than 800 pg/mL.

In some cases, a unit dose of the composition does not increase systemic levels of monocyte chemotactic protein-1 (MCP-1), macrophage inflammatory protein-1 (MIP-1β), interleukin-1 receptor agonist (IL-1RA), or a combination thereof by more than 20-fold above levels prior to the administering. In some cases, a unit dose of the composition does not increase systemic levels of monocyte chemotactic protein-1 (MCP-1), macrophage inflammatory protein-1 (MIP-1β), interleukin-1 receptor agonist (IL-1RA), or a combination thereof by more than 10-fold above levels prior to the administering. In some cases, a unit dose of the composition does not increase systemic levels of monocyte chemotactic protein-1 (MCP-1), macrophage inflammatory protein-1 (MIP-1β), interleukin-1 receptor agonist (IL-1RA), or a combination thereof by more than 5-fold above levels prior to the administering. In some cases, a unit dose of the composition does not increase systemic levels of monocyte chemotactic protein-1 (MCP-1), macrophage inflammatory protein-1 (MIP-1β), interleukin-1 receptor agonist (IL-1RA), or a combination thereof by more than 3-fold above levels prior to the administering.

In some cases, a unit dose of the composition does not increase systemic levels of MIP-1β by more than 20-fold above levels prior to the administering. In some cases, a unit dose of the composition does not increase systemic levels of MIP-1β by more than 10-fold above levels prior to the administering. In some cases, a unit dose of the composition does not increase systemic levels of MIP-1β by more than 5-fold above levels prior to the administering. In some cases, a unit dose of the composition does not increase systemic levels of MIP-43 by more than 3-fold above levels prior to the administering.

In some cases, a unit dose of the composition does not increase systemic levels of IL-1RA by more than 20-fold above levels prior to the administering. In some cases, a unit dose of the composition does not increase systemic levels of IL-1RA by more than 10-fold above levels prior to the administering. In some cases, a unit dose of the composition does not increase systemic levels of IL-1RA by more than 5-fold above levels prior to the administering. In some cases, a unit dose of the composition does not increase systemic levels of IL-1RA by more than 3-fold above levels prior to the administering.

In some embodiments, the composition comprises a first population comprising a distribution of MEF antibodies; a second population comprising a distribution of MEF antibodies; and at least one pharmaceutically acceptable carrier; wherein the BPMs present in the first population of MEF antibodies are different than the BPMs present in the second population of MEF antibodies. In some embodiments, the composition comprises a first population comprising a distribution of MEF antibodies; a second population comprising a distribution of MEF antibodies; and at least one pharmaceutically acceptable carrier; wherein the cleavable moieties present in the first population of MEF antibodies are different than the cleavable moieties present in the second population of MEF antibodies.

In some embodiments, the first population and the second population are substantially the same except for the BPMs. In some embodiments, the first population and the second population are substantially the same except for the cleavable moieties. For example, the first and second populations can have substantially the same distribution of MEF antibodies (i.e., the average number of BPMs per antibody), number of BPMs, and/or location of covalent linkage of one or more cleavable moieties to each MEF antibody.

In some embodiments, the first population and the second population are different, in addition to having different BPMs. In some embodiments, the first population and the second population are different, in addition to having different cleavable moieties. For example, the first and second populations can have different distributions of MEF antibodies, number of BPMs, and location of covalent linkage of one or more cleavable moieties to each MEF antibody.

In some embodiments, the percent aggregation of antibodies as described herein in the composition is increased by not more than about 1-fold to about 1.1 fold relative to an equivalent antibody lacking BPM functionalizations. In some embodiments, the percent aggregation of antibodies as described herein in the composition is increased by about 1-fold to about 1.1 fold relative to an equivalent antibody lacking BPM functionalizations. For example, the percent aggregation can be increased by about 1-fold, about 1.01-fold, about 1.02-fold, about 1.03-fold, about 1.04-fold, about 1.05-fold, about 1.06-fold, about 1.07-fold, about 1.08-fold, about 1.09-fold, about 1.1-fold, or any value in between, relative to an equivalent antibody lacking BPM functionalizations. In some embodiments, the percent aggregation is determined by spectrophotometric (e.g., OD) or chromatographic methods (e.g., SEC or HIC).

The preferred route of administration for the MEF antibody composition described herein is parenteral. Parenteral administration includes subcutaneous injections, intravenous, intramuscular, intrasternal injection or infusion techniques. In some embodiments, the compositions are administered parenterally. In one of those embodiments, the compositions are administered intravenously. Administration is typically through any convenient route, for example by infusion or bolus injection.

Pharmaceutical compositions of an antibody are formulated so as to allow it to be bioavailable upon administration of the composition to a subject. Compositions will be in the form of one or more injectable dosage units.

Materials used in preparing the pharmaceutical compositions can be non-toxic in the amounts used. It will be evident to those of ordinary skill in the art that the optimal dosage of the active ingredient(s) in the pharmaceutical composition will depend on a variety of factors. Relevant factors include, without limitation, the type of animal (e.g., human), the particular form of the compound, the manner of administration, and the composition employed.

In some embodiments, the MEF antibody composition described herein is a solid, for example, as a lyophilized powder, suitable for reconstitution into a liquid prior to administration. In some embodiments, the MEF antibody described herein composition is a liquid composition, such as a solution or a suspension. A liquid composition or suspension is useful for delivery by injection and a lyophilized solid is suitable for reconstitution as a liquid or suspension using a diluent suitable for injection. In a composition administered by injection, one or more of a surfactant, preservative, wetting agent, dispersing agent, suspending agent, buffer, stabilizer and isotonic agent is typically included.

In some embodiments the liquid compositions, whether they are solutions, suspensions or other like form, can also include one or more of the following: sterile diluents such as water for injection, saline solution, preferably physiological saline, Ringer's solution, isotonic sodium chloride, fixed oils such as synthetic mono or diglycerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as amino acids, acetates, citrates or phosphates; detergents, such as nonionic surfactants, polyols; and agents for the adjustment of tonicity such as sodium chloride or dextrose. A parenteral composition is typically enclosed in ampoule, a disposable syringe or a multiple-dose vial made of glass, plastic or other material. Physiological saline is an exemplary adjuvant. An injectable composition is preferably a liquid composition that is sterile.

The amount of an antibody as described herein that is effective in the treatment of a particular disorder or condition will depend on the nature of the disorder or condition, which is usually determined by standard clinical techniques. In addition, in vitro or in vivo assays are sometimes employed to help identify optimal dosage ranges. The precise dose to be employed in the compositions will also depend on the route of parenteral administration, and the seriousness of the disease or disorder, and should be decided according to the judgment of the practitioner and each subject's circumstances.

In some embodiments, the compositions comprise a therapeutically effective amount of an antibody as described herein such that a suitable dosage will be obtained. Typically, this amount is at least about 0.01% of the MEF antibody by weight of the composition.

In some embodiments, the compositions dosage of an antibody administered to a subject is from about 0.01 mg/kg to about 100 mg/kg, from about 1 to about 100 mg of a per kg or from about 0.1 to about 25 mg/kg of the subject's body weight. In some embodiments, the dosage administered to a subject is about 0.01 mg/kg to about 15 mg/kg of the subject's body weight. In some embodiments, the dosage administered to a subject is about 0.1 mg/kg to about 15 mg/kg of the subject's body weight. In some embodiments, the dosage administered to a subject is about 0.1 mg/kg to about 20 mg/kg of the subject's body weight. In some embodiments, the dosage administered is about 0.1 mg/kg to about 5 mg/kg or about 0.1 mg/kg to about 10 mg/kg of the subject's body weight. In some embodiments, the dosage administered is about 1 mg/kg to about 15 mg/kg of the subject's body weight. In some embodiments, the dosage administered is about 1 mg/kg to about 10 mg/kg of the subject's body weight. In some embodiments, the dosage administered is about 0.1 to about 4 mg/kg, about 0.1 to about 3.2 mg/kg, or about 0.1 to about 2.7 mg/kg of the subject's body weight over a treatment cycle.

The term “carrier” refers to a diluent, adjuvant or excipient, with which a compound is administered. Such pharmaceutical carriers are liquids. Water is an exemplary carrier when the compounds are administered intravenously. Saline solutions and aqueous dextrose and glycerol solutions are also useful as liquid carriers for injectable solutions. Suitable pharmaceutical carriers also include glycerol, propylene, glycol, or ethanol. The present compositions, if desired, will in some embodiments also contain minor amounts of wetting or emulsifying agents, and/or pH buffering agents.

In some embodiments, the antibodies described herein are formulated in accordance with routine procedures as a pharmaceutical composition adapted for intravenous administration to animals, particularly human beings. Typically, the carriers or vehicles for intravenous administration are sterile isotonic aqueous buffer solutions. In some embodiments, the composition further comprises a local anesthetic, such as lignocaine, to ease pain at the site of the injection. In some embodiments, an antibody as described herein and the remainder of the formulation are supplied either separately or mixed together in unit dosage form, for example, as a dry lyophilized powder or water free concentrate in a hermetically sealed container such as an ampoule or sachette indicating the quantity of active agent. Where an antibody is to be administered by infusion, it is sometimes dispensed, for example, with an infusion bottle containing sterile pharmaceutical grade water or saline. Where the composition is administered by injection, an ampoule of sterile water for injection or saline is typically provided so that the ingredients can be mixed prior to administration.

The pharmaceutical compositions are generally formulated as sterile, substantially isotonic and in full compliance with all Good Manufacturing Practice (GMP) regulations of the U.S. Food and Drug Administration.

### Methods of Use

Some embodiments provide a method of treating cancer in a subject in need thereof, comprising administering to the subject a therapeutically effective amount of a MEF antibody.

Some embodiments provide a method of treating cancer in a subject in need thereof, comprising administering to the subject a therapeutically effective amount of a MEF antibody to the subject before, during, or after administration of another anticancer agent to the subject.

Some embodiments provide a method for delaying and/or preventing acquired resistance to an anticancer agent, comprising administering a therapeutically effective amount of a MEF antibody to a subject at risk for developing or having acquired resistance to an anticancer agent. In some embodiments, the subject is administered a dose of the anticancer agent (e.g., at substantially the same time as a dose of a MEF antibody is administered to the subject).

Some embodiments provide a method of delaying and/or preventing development of cancer resistant to an anticancer agent in a subject, comprising administering to the subject a therapeutically effective amount of a MEF antibody before, during, or after administration of a therapeutically effective amount of the anticancer agent.

Some embodiments provide a method of treating a condition in a subject in need thereof, the method comprising: administering to the subject a therapeutically effective amount of a composition comprising a modulated effector function (MEF) antibody which comprises a modification which decreases an effector function of the MEF antibody, and which at least partially reverses subsequent to the administration to affect an increase in the effector function; and treating the condition while maintaining a systemic level of monocyte chemotactic protein-1 (MCP-1) to no more than 10-fold above a level prior to the administering. In some cases, the method further comprises maintaining levels of tumor necrosis factor (TNF-α), interferon gamma (IFN-γ), interleukin 1 beta (IL1B), interleukin 6 (IL6), interleukin 10 (IL10), or a combination thereof to no more than 10-fold above levels prior to the administering. In some cases, the modification comprises a cleavable biocompatible polymeric moiety (BPM) covalently attached to an amino acid residue or a post-translational modification of the MEF antibody. In some cases, prior to the BPM cleavage, the MEF antibody has between 2% and 20% of the effector function activity of an equivalent antibody lacking the BPM. In some cases, 192 hours after administration, the MEF antibody has between 30% and 70% of the effector function activity of an equivalent antibody lacking the BPM. In some cases, the modification which decreases the effector function of the MEF antibody decreases FcγRIII binding affinity of the MEF antibody.

The antibodies described herein are useful for inhibiting the multiplication of a tumor cell or cancer cell, causing apoptosis in a tumor or cancer cell, and/or for treating cancer in a subject in need thereof. The antibodies can be used accordingly in a variety of settings for the treatment of cancers.

In some embodiments, a MEF antibody as described herein binds to the tumor cell or cancer cell. In some embodiments, a MEF antibody as described herein binds to a tumor cell or cancer cell antigen which is on the surface of the tumor cell or cancer cell. In some embodiments, a MEF antibody as described herein binds to a tumor cell or cancer cell antigen which is an extracellular matrix protein associated with the tumor cell or cancer cell.

The specificity of the MEF antibody for a particular tumor cell or cancer cell can be important for determining those tumors or cancers that are most effectively treated. For example, antibodies that target a cancer cell antigen present on hematopoietic cancer cells in some embodiments treat hematologic malignancies. In some embodiments, antibodies that target a cancer cell antigen present on abnormal cells of solid tumors for treating such solid tumors. In some embodiments, antibodies are directed against abnormal cells of hematopoietic cancers such as, for example, lymphomas (Hodgkin Lymphoma and Non-Hodgkin Lymphomas) and leukemias and solid tumors.

Cancers, including, but not limited to, a tumor, metastasis, or other disease or disorder characterized by abnormal cells that are characterized by uncontrolled cell growth in some embodiments are treated or inhibited by administration of a MEF antibody.

In some embodiments, the subject has previously undergone treatment for the cancer. In some embodiments, the prior treatment is surgery, radiation therapy, administration of one or more anticancer agents, or a combination of any of the foregoing.

In any of the methods described herein, the cancer is selected from the group consisting of: adenocarcinoma, adrenal gland cortical carcinoma, adrenal gland neuroblastoma, anus squamous cell carcinoma, appendix adenocarcinoma, bladder urothelial carcinoma, bile duct adenocarcinoma, bladder carcinoma, bladder urothelial carcinoma, bone chordoma, bone marrow leukemia lymphocytic chronic, bone marrow leukemia non-lymphocytic acute myelocytic, bone marrow lymph proliferative disease, bone marrow multiple myeloma, bone sarcoma, brain astrocytoma, brain glioblastoma, brain medulloblastoma, brain meningioma, brain oligodendroglioma, breast adenoid cystic carcinoma, breast carcinoma, breast ductal carcinoma in situ, breast invasive ductal carcinoma, breast invasive lobular carcinoma, breast metaplastic carcinoma, cervix neuroendocrine carcinoma, cervix squamous cell carcinoma, colon adenocarcinoma, colon carcinoid tumor, duodenum adenocarcinoma, endometrioid tumor, esophagus adenocarcinoma, esophagus and stomach carcinoma, eye intraocular melanoma, eye intraocular squamous cell carcinoma, eye lacrimal duct carcinoma, fallopian tube serous carcinoma, gallbladder adenocarcinoma, gallbladder glomus tumor, gastroesophageal junction adenocarcinoma, head and neck adenoid cystic carcinoma, head and neck carcinoma, head and neck neuroblastoma, head and neck squamous cell carcinoma, kidney chromophore carcinoma, kidney medullary carcinoma, kidney renal cell carcinoma, kidney renal papillary carcinoma, kidney sarcomatoid carcinoma, kidney urothelial carcinoma, kidney carcinoma, leukemia lymphocytic, leukemia lymphocytic chronic, liver cholangiocarcinoma, liver hepatocellular carcinoma, liver carcinoma, lung adenocarcinoma, lung adenosquamous carcinoma, lung atypical carcinoid, lung carcinosarcoma, lung large cell neuroendocrine carcinoma, lung non-small cell lung carcinoma, lung sarcoma, lung sarcomatoid carcinoma, lung small cell carcinoma, lung small cell undifferentiated carcinoma, lung squamous cell carcinoma, upper aerodigestive tract squamous cell carcinoma, upper aerodigestive tract carcinoma, lymph node lymphoma diffuse large B cell, lymph node lymphoma follicular lymphoma, lymph node lymphoma mediastinal B-cell, lymph node lymphoma plasmablastic lung adenocarcinoma, lymphoma follicular lymphoma, lymphoma, non-Hodgkins, nasopharynx and paranasal sinuses undifferentiated carcinoma, ovary carcinoma, ovary carcinosarcoma, ovary clear cell carcinoma, ovary epithelial carcinoma, ovary granulosa cell tumor, ovary serous carcinoma, pancreas carcinoma, pancreas ductal adenocarcinoma, pancreas neuroendocrine carcinoma, peritoneum mesothelioma, peritoneum serous carcinoma, placenta choriocarcinoma, pleura mesothelioma, prostate acinar adenocarcinoma, prostate carcinoma, rectum adenocarcinoma, rectum squamous cell carcinoma, skin adnexal carcinoma, skin basal cell carcinoma, skin melanoma, skin Merkel cell carcinoma, skin squamous cell carcinoma, small intestine adenocarcinoma, small intestine gastrointestinal stromal tumors (GISTs), large intestine/colon carcinoma, large intestine adenocarcinoma, soft tissue angiosarcoma, soft tissue Ewing sarcoma, soft tissue hemangioendothelioma, soft tissue inflammatory myofibroblastic tumor, soft tissue leiomyosarcoma, soft tissue liposarcoma, soft tissue neuroblastoma, soft tissue paraganglioma, soft tissue perivascular epithelioid cell tumor, soft tissue sarcoma, soft tissue synovial sarcoma, stomach adenocarcinoma, stomach adenocarcinoma diffuse-type, stomach adenocarcinoma intestinal type, stomach adenocarcinoma intestinal type, stomach leiomyosarcoma, thymus carcinoma, thymus thymoma lymphocytic, thyroid papillary carcinoma, unknown primary adenocarcinoma, unknown primary carcinoma, unknown primary malignant neoplasm, lymphoid neoplasm, unknown primary melanoma, unknown primary sarcomatoid carcinoma, unknown primary squamous cell carcinoma, unknown undifferentiated neuroendocrine carcinoma, unknown primary undifferentiated small cell carcinoma, uterus carcinosarcoma, uterus endometrial adenocarcinoma, uterus endometrial adenocarcinoma endometrioid, uterus endometrial adenocarcinoma papillary serous, and uterus leiomyosarcoma.

In some embodiments, the subject is concurrently administered one or more additional anticancer agents with a MEF antibody. In some embodiments, the subject is concurrently receiving radiation therapy with a MEF antibody. In some embodiments, the subject is administered one or more additional anticancer agents after administration of a MEF antibody. In some embodiments, the subject receives radiation therapy after administration of a MEF antibody.

In some embodiments, the subject has discontinued the prior therapy, for example, due to unacceptable or unbearable side effects, where the prior therapy was too toxic, and/or where the subject developed resistance to the prior therapy.

Some embodiments provide a method of treating an autoimmune disorder in a subject in need thereof, comprising administering to the subject a therapeutically effective amount of a MEF antibody.

Some embodiments provide a method of treating an autoimmune disorder in a subject in need thereof, comprising administering a therapeutically effective amount of a MEF antibody to the subject before, during, or after administration of an additional therapeutic agent to the subject (e.g., methotrexate).

Some embodiments provide a method of ameliorating one or more symptoms of an autoimmune disorder in a subject in need thereof, comprising administering to the subject a therapeutically effective amount of a MEF antibody.

Some embodiments provide a method of ameliorating one or more symptoms of an autoimmune disorder in a subject in need thereof, comprising administering to the subject a therapeutically effective amount of a MEF antibody before, during, or after administration of an additional therapeutic agent to the subject.

Some embodiments provide a method of reducing the occurrence of flare-ups of an autoimmune disorder in a subject in need thereof, comprising administering to the subject a therapeutically effective amount of a MEF antibody.

Some embodiments provide a method of reducing the occurrence of flare-ups an autoimmune disorder in a subject in need thereof, comprising administering to the subject a therapeutically effective amount of a MEF antibody before, during, or after administration of an additional therapeutic agent to the subject (e.g., methotrexate).

A “flare-up” refers to a sudden onset of symptoms, or sudden increase in severity of symptoms, of a disorder. For example, a flare-up in mild joint pain typically addressed with non-steroidal anti-inflammatory drugs (NSAIDs) could result in debilitating joint pain, preventing normal locomotion even with NSAIDS.

In some embodiments, a MEF antibody as described herein binds to an autoimmune antigen. In some embodiments, the antigen is on the surface of a cell involved in an autoimmune disorder. In some embodiments, a MEF antibody as described herein binds to an autoimmune antigen which is on the surface of a cell. In some embodiments, a MEF antibody as described herein binds to activated lymphocytes that are associated with the autoimmune disorder state. In some embodiments, the kills or inhibit the multiplication of cells that produce an autoimmune antibody associated with a particular autoimmune disorder.

In some embodiments, the subject is concurrently administered one or more additional therapeutic agents with a MEF antibody as described herein. In some embodiments, one or more additional therapeutic agents are compounds known to treat and/or ameliorate the symptoms of an autoimmune disorder (e.g., compounds that are approved by the FDA or EMA for the treatment of an autoimmune disorder).

In some embodiments, the autoimmune disorders include, but are not limited to, Th2 lymphocyte related disorders (e.g., atopic dermatitis, atopic asthma, rhinoconjunctivitis, allergic rhinitis, Omenn's syndrome, systemic sclerosis, and graft versus host disease); Th1 lymphocyte-related disorders (e.g., rheumatoid arthritis, multiple sclerosis, psoriasis, Sjorgren's syndrome, Hashimoto's thyroiditis, Grave's disease, primary biliary cirrhosis, Wegener's granulomatosis, and tuberculosis); and activated B lymphocyte-related disorders (e.g., systemic lupus erythematosus, Goodpasture's syndrome, rheumatoid arthritis, and type I diabetes).

In some embodiments, the one or more symptoms of an autoimmune disorder include, but are not limited to joint pain, joint swelling, skin rash, itching, fever, fatigue, anemia, diarrhea, dry eyes, dry mouth, hair loss, and muscle aches.

Infusion related reactions associated with administration of antibodies are graded by increasing severity from 0 (no reaction) to 4 (severe reaction). Subjects with a Grade 1 or 2 infusion related reaction exhibit mild symptoms, and subjects with a Grade 3 reaction exhibit moderate symptoms. Some embodiments provide a method of decreasing the severity of an infusion related reaction associated with an antibody, comprising intravenously administering a composition comprising the MEF antibodies described herein to a subject in need thereof; wherein the severity of the infusion related reaction is decreased from 1 to 4 units relative to intravenous administration of an equimolar amount of the antibody, wherein the antibody is equivalent to the MEF antibody. In some embodiments, the severity is decreased by 1 unit, by 2 units, by 3 units, or by 4 units, to a minimum score of 0, for example, a maximum decrease of Grade 4 to Grade 0.

Some embodiments provide a method of reducing the incidence of and/or risk of developing an infusion related reaction associated with an antibody, comprising intravenously administering a composition comprising the MEF antibodies described herein to a subject in need thereof; wherein the incidence the infusion related reaction is reduced relative to intravenous administration of an equimolar amount of the antibody, and wherein the antibody is equivalent to the MEF antibody. In some embodiments, the incidence and/or risk is reduced by about 10% to about 99%, for example, about 10% to about 50%, about 25% to about 75%, about 50% to about 99%, or any value in between.

Some embodiments provide a method of reducing the symptoms of an infusion related reaction associated with an antibody, comprising intravenously administering a composition comprising the MEF antibodies described herein to a subject in need thereof; wherein the symptoms of the infusion related reaction are reduced relative to intravenous administration of an equimolar amount of the antibody, and wherein the antibody is equivalent to the MEF antibody. In some embodiments, reducing the symptoms of an infusion related reaction comprises reducing the number and/or severity of one or more symptoms. In some embodiments, the severity of one or more symptoms of an infusion related reaction is reduced by about 10% to about 99%, for example, about 10% to about 50%, about 25% to about 75%, about 50% to about 99%, or any value in between. In some embodiments, the one or more symptoms comprise nausea, vomiting, headache, tachycardia, hypotension, rash, flushing, fever, shortness of breath, bronchiospasm, urticaria, edema, or a combination of any of the foregoing.

Some embodiments provide a method of reducing the severity of an injection site reaction associated with an antibody, comprising intravenously administering a composition comprising the MEF antibodies described herein to a subject in need thereof; wherein the severity of the injection site reaction is reduced relative to intravenous administration of an equimolar amount of the antibody, and wherein the antibody is equivalent to the MEF antibody. In some embodiments, the severity of an injection site reaction is reduced by about 10% to about 99%, for example, about 10% to about 50%, about 25% to about 75%, about 50% to about 99%, or any value in between.

Some embodiments provide a method of reducing the symptoms of an injection site reaction associated with an antibody, comprising administering a composition comprising the MEF antibodies described herein to a subject in need thereof; wherein the symptoms of the injection site reaction are reduced relative to intravenous administration of an equimolar amount of the antibody, and wherein the antibody is equivalent to the MEF antibody. In some embodiments, the severity of one or more symptoms of an injection site reaction is reduced by about 10% to about 99%, for example, about 10% to about 50%, about 25% to about 75%, about 50% to about 99%, or any value in between. In some embodiments, the one or more symptoms comprise one or more of the following at the injection site: pain, itchiness, redness, burning, tenderness, warmth, blistering, or a combination of any of the foregoing.

Some embodiments provide a method of reducing the incidence of and/or risk of developing an injection site reaction associated with an antibody, comprising a composition comprising the antibodies described herein to a subject in need thereof; wherein the incidence of an injection site reaction is reduced relative to intravenous administration of an equimolar amount of the antibody, and wherein the antibody is equivalent to the MEF antibody. In some embodiments, the incidence and/or risk is reduced by about 10% to about 99%, for example, about 10% to about 50%, about 25% to about 75%, about 50% to about 99%, or any value in between.

Some embodiments provide a method of decreasing the Cₘₐₓ of an active antibody, comprising intravenously administering a composition comprising a distribution of MEF antibodies; wherein the active antibody is equivalent to the MEF antibody; and wherein the Cₘₐₓ of the active antibody after intravenous administration of the MEF antibody composition is decreased relative to the Cₘₐₓ after intravenous administration of an equimolar amount of the active antibody.

As used herein, an antibody that is an “active antibody” is an antibody that has substantially the same activity as an equivalent antibody. Active antibodies include antibodies that lack any remnant of the cleavable moieties and/or BPMs, as well as antibodies with one or more adducts of the cleavable moieties and/or BPMs still covalently attached. Despite their covalent attachment to the MEF antibody, these adducts have no meaningful impact on the efficacy of the MEF antibody. These adducts can be, for example, from cleavage of a particular cleavable moiety that will necessarily form such an adduct, from incomplete cleavage of one or more cleavable moieties, or from a secondary or alternative cleavage mechanism. In some embodiments, the active antibody comprises no remnant of the cleavable moieties and no remnant of the BPMs. In some embodiments, the active antibody comprises one or more adducts from the cleavable moieties and/or the BPMs. In some embodiments, the one or more adducts comprises 1-8 adducts from the cleavable moieties. In some embodiments, the one or more adducts comprises 2-4, 4-6, or 6-8 adducts from the cleavable moieties.

Some embodiments provide a method of delaying maximal Fc gamma receptor IIIa binding of an antibody, comprising intravenously administering a composition comprising the MEF antibodies described herein; wherein the antibody is equivalent to the MEF antibody; and wherein the MEF antibody delays binding to Fc gamma receptor IIIa relative to the antibody. In some embodiments, the delay in Fc gamma receptor IIIa a binding is about 3 hours to about 96 hours, for example, about 3 hours to about 12 hours, about 6 hours to about 18 hours, about 12 hours to about 24 hours, about 18 hours to about 36 hours, about 24 hours to about 48 hours, about 36 hours to about 72 hours, about 48 hours to about 96 hours, or any value in between. In some embodiments, the delay in Fc gamma receptor IIIa binding relative to an equivalent antibody is about 1.5-fold to about 50-fold, for example, about 1.5-fold to about 5-fold, about 3-fold to about 10-fold, about 6-fold to about 15-fold, about 10-fold to about 20-fold, about 15-fold to about 25-fold, about 20-fold to about 30-fold, about 25-fold to about 35-fold, about 30-fold to about 40-fold, about 35-fold to about 45-fold, about 40-fold to about 50-fold, or any value in between.

Some embodiments provide a method of selectively increasing binding of an antibody to Fc gamma receptor IIIa in a target cell in a subject, comprising intravenously administering to the subject a composition comprising the MEF antibodies described herein; wherein the antibody is equivalent to the MEF antibody; and wherein the ratio of the MEF antibody (i) bound to Fc gamma receptor IIIa at the target cell and (ii) bound to Fc gamma receptor IIIa systemically is increased relative to the ratio of the antibody (i) bound to Fc gamma receptor IIIa at the target cell and (ii) bound to Fc gamma receptor IIIa systemically. In some embodiments, the selective increase in Fc gamma receptor IIIa binding in a target cell relative to Fc gamma receptor IIIa binding systemically is about 1.5-fold to about 50-fold, for example, about 1.5-fold to about 5-fold, about 3-fold to about 10-fold, about 6-fold to about 15-fold, about 10-fold to about 20-fold, about 15-fold to about 25-fold, about 20-fold to about 30-fold, about 25-fold to about 35-fold, about 30-fold to about 40-fold, about 35-fold to about 45-fold, about 40-fold to about 50-fold, or any value in between.

Cytokine release syndrome is a systemic inflammatory response that can be triggered by administration of antibody immunotherapy resulting, in part, from off-target engagement of Fc receptors. Some embodiments provide a method of reducing systemic Fc activation in a subject after administration of an antibody, comprising intravenously administering to the subject a composition comprising the antibodies described herein; wherein the MEF antibody reduces systemic activation of Fc relative to an equivalent antibody. In some embodiments, systemic activation of Fc is reduced by about 10% to about 100% (elimination of systemic activation of Fc relative to an equivalent antibody). In some embodiments, systemic activation of Fc is reduced by about 10% to about 50%, about 30% to about 70%, about 50% to about 90%, about 70% to about 100%, or any value in between.

Some embodiments provide a method of reducing systemic Fc gamma receptor IIIa activation in a subject after administration of an antibody, comprising intravenously administering to the subject a composition comprising the MEF antibodies described herein; wherein the antibody is equivalent to the MEF antibody; and wherein the administration of the MEF antibody provides reduced systemic activation of Fc gamma receptor IIIa relative to intravenous administration of an equimolar amount of the antibody. In some embodiments, systemic activation of Fc gamma receptor IIIa is reduced by about 10% to about 100% (elimination of systemic activation of Fc gamma receptor IIIa relative to an equivalent antibody). In some embodiments, systemic activation of Fc gamma receptor IIIa is reduced by about 10% to about 50%, about 30% to about 70%, about 50% to about 90%, about 70% to about 100%, or any value in between.

Some embodiments provide a method of decreasing systemic cytokine production in a subject after administration of an antibody, comprising intravenously administering to the subject a composition comprising the MEF antibodies described herein; wherein the antibody is equivalent to the MEF antibody; and wherein administration of the composition comprising the MEF antibody decreases systemic cytokine production relative to intravenous administration of an equimolar amount of the antibody. In some embodiments, the systemic cytokine levels in the plasma of the subject are reduced by about 1% to about 80%. For example, about 1% to about 20%, about 10% to about 30%, about 20% to about 40%, about 30% to about 50%, about 40% to about 60%, about 50% to about 70%, about 60% to about 80%, or any value in between.

Some embodiments provide a method of selectively activating an antibody, comprising intravenously administering a composition comprising a distribution of MEF antibodies as described herein to a subject; wherein at least about 10% of the BPMs are cleaved from the MEF antibody within about 12 hours and at least about 25% of the BPMs are cleaved from the MEF antibody within 48 hours; wherein at least about 10% of the BPMs are cleaved from the MEF antibody within about 12 hours and at least about 30% of the BPMs are cleaved from the MEF antibody within 48 hours; wherein at least about 20% of the BPMs are cleaved from the MEF antibody within about 12 hours; and at least about 40% of the BPMs are cleaved from the MEF antibody within 48 hours; wherein at least about 30% of the BPMs are cleaved from the MEF antibody within about 12 hours and at least about 50% of the BPMs are cleaved from the MEF antibody within 48 hours; wherein at least about 50% of the BPMs are cleaved from the MEF antibody within about 12 hours and at least about 75% of the BPMs are cleaved from the MEF antibody within 48 hours; wherein at least about 50% of the BPMs are cleaved from the MEF antibody within about 12 hours and at least about 100% of the BPMs are cleaved from the MEF antibody within 48 hours.

In some embodiments, each cleavable moiety comprises a structure according to Formula (II):

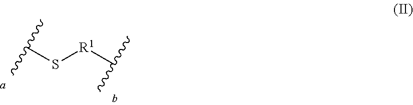

wherein at least about 10% of the BPMs are cleaved from the MEF antibody within about 12 hours and at least about 25% of the BPMs are cleaved from the MEF antibody within 48 hours after intravenous administration.

In some embodiments, each cleavable moiety comprises a structure according to Formula (III):

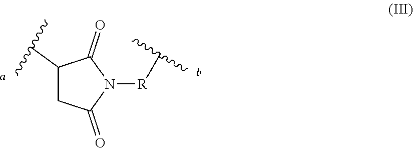

wherein at least about 10% of the BPMs are cleaved from the MEF antibody within about 12 hours and at least about 25% of the BPMs are cleaved from the MEF antibody within 48 hours after intravenous administration.

In some embodiments, at least about 10% of the BPMs are cleaved from the MEF antibody within about 12 hours and at least about 30% of the BPMs are cleaved from the MEF antibody within 48 hours after intravenous administration to a subject. In some embodiments, at least about 20% of the BPMs are cleaved from the MEF antibody within about 12 hours and at least about 40% of the BPMs are cleaved from the MEF antibody within 48 hours after intravenous administration to a subject. In some embodiments, at least about 30% of the BPMs are cleaved from the MEF antibody within about 12 hours and at least about 50% of the BPMs are cleaved from the MEF antibody within 48 hours after intravenous administration to a subject. In some embodiments, at least about 50% of the BPMs are cleaved from the MEF antibody within about 12 hours and about 100% of the BPMs are cleaved from the MEF antibody within 48 hours after intravenous administration to a subject. In some embodiments, at least about 50% of the BPMs are cleaved from the MEF antibody within about 12 hours to a subject. In some embodiments, cleavage of one or more cleavable moieties (and thus, removal of the BPM from the antibody) releases an active antibody, as described herein.

In some embodiments, each cleavable moiety comprises a structure according to Formula (II):

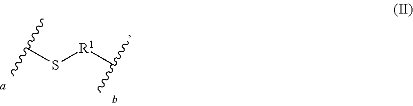

wherein at least about 50% of the BPMs are cleaved from the MEF antibody within about 12 hours and about 100% of the BPMs are cleaved from the MEF antibody within 48 hours after intravenous administration to a subject.

In some embodiments, each cleavable moiety comprises a structure according to Formula (III):

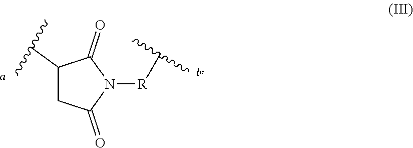

wherein at least about 30% of the BPMs are cleaved from the MEF antibody within about 12 hours and at least about 50% of the BPMs are cleaved from the MEF antibody within 48 hours after intravenous administration to a subject.

In some embodiments, each cleavable moiety comprises a structure according to Formula (III):

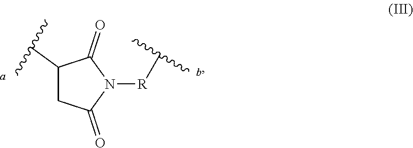

wherein about 30% to about 50% of the BPMs are cleaved from the MEF antibody within about 12 hours and about 50% to about 75% of the BPMs are cleaved from the MEF antibody within 48 hours after intravenous administration to a subject.

In some embodiments, the MEF antibody is modified with a cleavable moiety comprising a maleimido group of different carbon chain lengths, for example, a 3-carbon chain (maleimidopropionyl), a 6-carbon chain (maleimidocaproyl), a 7-carbon chain (maleimidoheptanoyl), or an 8-carbon chain (maleimidooctanoyl). In some embodiments, the MEF antibody is modified with a cleavable moiety comprising a maleimidopropionyl group. In some embodiments, the MEF antibody is modified with a cleavable moiety comprising a maleimidocaproyl group. In some embodiments, the MEF antibody is modified with a cleavable moiety comprising a maleimidocaproyl group covalently linked to a PEG group of different number of ethylene glycol units, for example, a 2-ethylene glycol unit PEG (PEG4), a 4-ethylene glycol unit PEG (PEGS), a 6-ethylene glycol unit PEG (PEG12), an 18-ethylene glycol unit PEG (PEG36), or a 24-ethylene glycol unit PEG (PEG48). In some embodiments, the MEF antibody is modified with a cleavable moiety comprising a maleimidocaproyl group covalently linked to a PEG12 group. In some embodiments, the MEF antibody is modified with a cleavable moiety comprising a maleimidocaproyl group covalently linked to a PEG48 group. In some embodiments, the MEF antibody is modified with a cleavable moiety having the structure according to either Formula (IIo) or (IIIb):

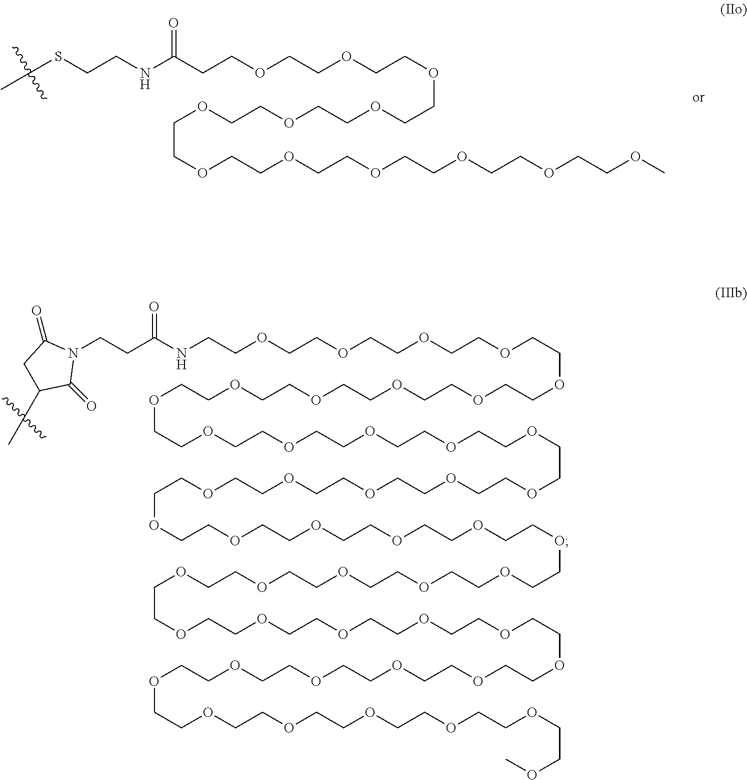

wherein [symbol] represents the covalent attachment to a sulfur atom of the antibody (e.g., the sulfur atom of a cysteine residue of a reduced interchain disulfide bond of the MEF antibody).

In some embodiments, the MEF antibody is modified with a cleavable moiety comprising a branched structure. As disclosed herein, many branched polymers (in particular branched PEG polymers) comprise lower hydrodynamic radii and intrinsic viscosities than equivalent molecular weight linear polymers. These properties can, in certain cases, be exploited to generate MEF antibodies with greater steric shielding at sites surrounding BPM attachment (e.g., antibody Fc regions) and properties (e.g., diffusion, biological partitioning, etc.) more closely mimicking the non-BPM-modified antibody. Furthermore, in some cases, BPM branching structure affects cleavable moiety (e.g., disulfide attachment) accessibility, thereby modifying and/or increasing control over BPM cleavage rate. In some cases, the BPM comprises at least two branches, such as the (PEG4)₂ of MEF antibody Anti-CD40-AF-17 (outlined in Example 5). In some cases, the BPM comprises at least three branches, such as the PEG4-(PEG8)₃ of MEF antibody Anti-CD40-AF-19 (outlined in Example 5).

In some embodiments, the MEF antibody is modified with a cleavable moiety comprising a disulfide group covalently linked to a branched or linear carbon chain of different lengths, for example, a linear 2-carbon chain, a branched 2-carbon chain, a linear 3-carbon chain, a linear 4-carbon chain, or a linear 5-carbon chain. In some embodiments, the MEF antibody is modified with a cleavable moiety comprising a disulfide group covalently linked to a branched or linear 2-carbon chain. In some embodiments, the MEF antibody is modified with a cleavable moiety comprising a disulfide group covalently linked to a branched or linear 2-carbon chain, which is further covalently linked to a PEG group of different number of ethylene glycol units, for example, a 2-ethylene glycol unit PEG (PEG4), a 4-ethylene glycol unit PEG (PEGS), a 6-ethylene glycol unit PEG (PEG12), an 18-ethylene glycol unit PEG (PEG36), or a 24-ethylene glycol unit PEG (PEG48). In some embodiments, the cleavable moiety comprises a disulfide group covalently linked to a linear 2-carbon chain which is further covalently linked to a PEG12 group. In some embodiments, the MEF antibody is modified with a cleavable moiety having a structure according to Formula (IIo):

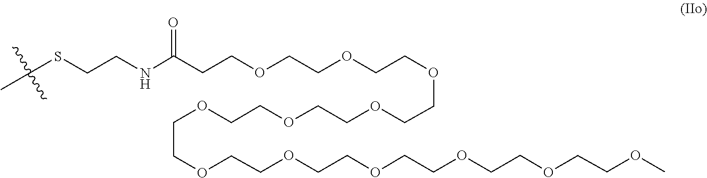

wherein [symbol] represents the covalent attachment to a sulfur atom of the antibody (e.g., the sulfur atom of a cysteine residue of a reduced interchain disulfide bond of the MEF antibody).

In some embodiments, the cleavable moiety comprises a disulfide linkage and about 10% to about 50% of the BPMs are released within 12 hours, for example, about 10% to about 30%, about 20% to about 40%, or about 30% to about 50%. In some embodiments, the cleavable moiety comprises a disulfide linkage and about 25% to about 100% of the BPMs are released within 24 hours, for example, about 25% to about 50%, about 40% to about 60%, about 50% to about 70%, about 60% to about 80%, about 70% to about 90%, or about 80% to about 100%. In some embodiments, the cleavable moiety comprises a disulfide linkage and about 25% to about 100% of the BPMs are released within 48 hours, for example, about 25% to about 50%, about 40% to about 60%, about 50% to about 70%, about 60% to about 80%, about 70% to about 90%, or about 80% to about 100%.

In some embodiments, the cleavable moiety comprises a succinimide moiety, and about 25 to about 75% are released within 24 hours, for example, about 25% to about 45%, about 35% to about 55%, about 45% to about 65%, or about 55% to about 75%. In some embodiments, cleavable moiety comprises a succinimide moiety, and about 25 to about 75% are released within 48 hours, for example, about 25% to about 45%, about 35% to about 55%, about 45% to about 65%, or about 55% to about 75%. In some embodiments, the cleavable moiety comprises a succinimide moiety, and about 50 to about 100% are released within 96 hours, for example, about 50% to about 70%, about 60% to about 80%, about 70% to about 90%, or about 80% to about 100%.

### EXAMPLES

### General Methods

All commercially available anhydrous solvents and reagents were used without further purification. UPLC-MS system 1 consisted of a Waters SQ mass detector 2 interfaced to an Acquity Ultra Performance LC equipped with a CORTECS UPLC C18 2.1×50 mm, 1.6 μm reverse phase column (Method 1). The acidic mobile phase (0.1% formic acid) consisted of a gradient of 3% acetonitrile/97% water to 100% acetonitrile (flow rate=0.5 mL/min). UPLC-MS system 2 consisted of a Waters Xevo G2 ToF mass spectrometer interfaced to a Waters Acquity H-Class Ultra Performance LC equipped with a CORTECS UPLC C18 2.1×50 mm, 1.6 μm reverse phase column (Method 2). Reaction monitoring was performed by PLRP-MS (Poly LC reverse phase HPLC with electrospray ionization QTOF mass spectroscopy). Microwave reactions were conducted in a Biotage Initiator+ microwave reactor. Preparative HPLC was carried out on a Waters 2545 Binary Gradient Module with a Waters 2998 Photodiode Array Detector. Products were purified over a C12 Phenomenex Synergi 250×10.0 mm, 4 μm, 80 Å reverse phase column (<10 mg scale) (Method 3) or a C12 Phenomenex Synergi 250×50 mm, 10 Inn, 80 Å reverse phase column (10-100 mg scale) (Method 4) eluting with 0.1% (v/v) trifluoroacetic acid (TFA) in water (solvent A) and 0.1% (v/v) TFA in acetonitrile (MeCN) (solvent B). The purification methods generally consisted of linear gradients of solvent A to solvent B, ramping from 90% aqueous solvent A to 5% solvent A. The flow rate was 4.6 mL/min with monitoring at UV 220 nm. NMR spectral data were collected on a Varian Mercury 400 MHz spectrometer. Coupling constants (1) are reported in hertz.

### Example 1

### Synthesis of Formamidine Disulfides

### Synthesis of 38-oxo-2,5,8,11,14,17,20,23,26,29,32,35-dodecaoxa-39-azahentetracontan-41-yl carbamo(dithioperoxo)imidate (1)

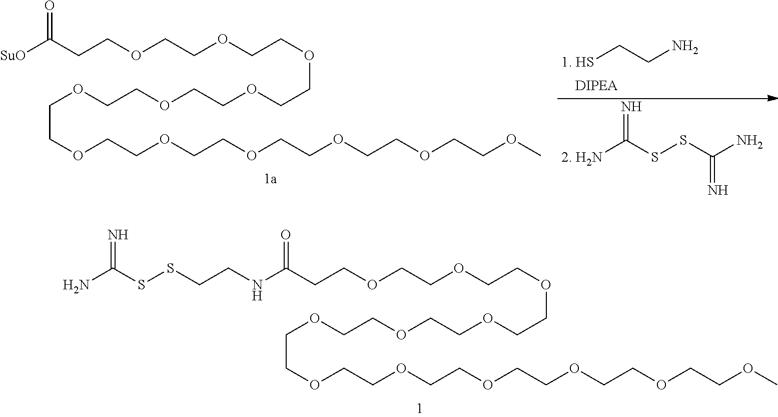

A 4-mL vial equipped with a stir bar was charged with PEG12-OSu ester (1a, 23.4 mg, 0.03 mmol), cystamine (4 mg, 0.05 mmol), diisopropylethylamine (DIPEA, 11.9 μL, 0.07 mmol) and N,N-dimethylformamide (DMF, 300 μL). The reaction mixture was stirred for 4 h at room temperature (RT). The reaction mixture was then concentrated in vacuo and the resulting residue re-dissolved in water (500 μL). Formamidine disulfide dihydrochloride (22 mg, 0.1 mmol) was added to the reaction mixture and the mixture was stirred for 3 h. The reaction mixture was then diluted with DMSO (2 mL) and water (2 mL) and loaded onto a preparative HPLC (Method 3) to isolate compound 1 (3 mg, 14% yield). Analytical UPLC-MS (Method 1): Retention time=0.97 min, m/z (ES+) (M+H)⁺, 722.35 (theoretical); 722.93 (observed).

### Synthesis of 38-oxo-2,5,8,11,14,17,20,23,26,29,32,35-dodecaoxa-39-azadotetracontan-41-yl carbamo(dithioperoxo)imidate (2)

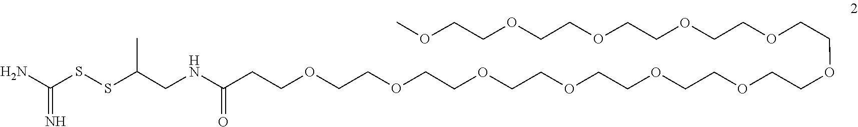 [SMILES: COCCOCCOCCOCCOCCOCCOCCOCCOCCOCCOCCOCCC(=O)NCC(C)SSC(=N)N]

Compound 2 was prepared using similar procedures as those used for compound 1, replacing cystamine with 1-aminopropane-2-thiol. Compound 2 was isolated using preparative HPLC (Method 3) (3 mg, 12% yield). Analytical UPLC-MS (Method 1): Retention time=1.15 min, m/z (ES+) (M+H)⁺, 736.37 (theoretical); 736.75 (observed).

### Synthesis of 38-oxo-2,5,8,11,14,17,20,23,26,29,32,35-dodecaoxa-39-azadotetracontan-42-yl carbamo(dithioperoxo)imidate (3)

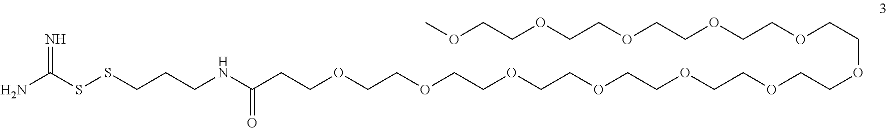 [SMILES: COCCOCCOCCOCCOCCOCCOCCOCCOCCOCCOCCOCCC(=O)NCCCSSC(=N)N]

Compound 3 was prepared using similar procedures as those used for compound 1, replacing cystamine with 3-aminopropane-1-thiol. Compound 3 was isolated using preparative HPLC (Method 3) (4 mg, 20% yield). Analytical UPLC-MS (Method 1): Retention time=1.04 min, m/z (ES+) (M+H)⁺: 736.37 (theoretical); 736.65 (observed).

### Synthesis of 38-oxo-2,5,8,11,14,17,20,23,26,29,32,35-dodecaoxa-39-azatritetracontan-43-yl carbamo(dithioperoxo)imidate (4)

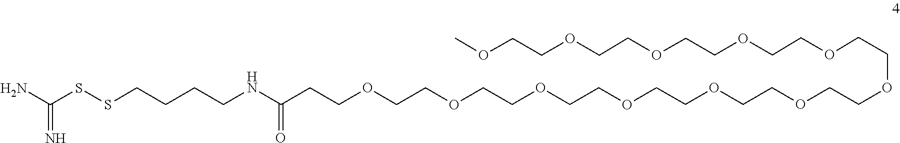 [SMILES: COCCOCCOCCOCCOCCOCCOCCOCCOCCOCCOCCOCCC(=O)NCCCCSSC(=N)N]

Compound 4 was prepared using similar procedures as those used for compound 1, replacing cystamine with 4-aminobutane-1-thiol. Compound 4 was isolated using preparative HPLC (Method 3) (4 mg, 18% yield). Analytical UPLC-MS (Method 1): Retention time=1.05 min, m/z (ES+) (M+H)⁺: 750.38 (theoretical); 750.41 (observed).

### Synthesis of 38-oxo-2,5,8,11,14,17,20,23,26,29,32,35-dodecaoxa-39-azatetratetracontan-44-yl carbamo(dithioperoxo)imidate (5)

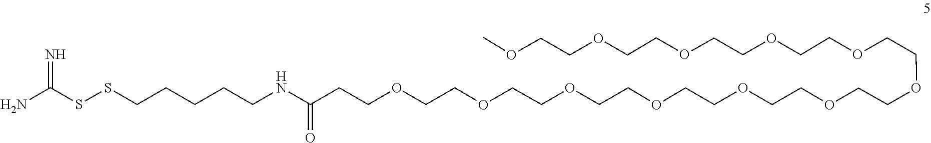 [SMILES: COCCOCCOCCOCCOCCOCCOCCOCCOCCOCCOCCOCCC(=O)NCCCCCSSC(=N)N]

Compound 5 was prepared using similar procedures as those used for compound 1, replacing cystamine with 5-aminopentane-1-thiol. Compound 5 was isolated using preparative HPLC (Method 3) (2 mg, 8% yield). Analytical UPLC-MS (Method 1): Retention time=1.06 min, m/z (ES+) (M+H)⁺: 764.40 (theoretical); 764.93 (observed).

### Synthesis of 38-oxo-2,5,8,11,14,17,20,23,26,29,32,35-dodecaoxa-39-azatetratetracontan-44-yl carbamo(dithioperoxo)imidate (6)

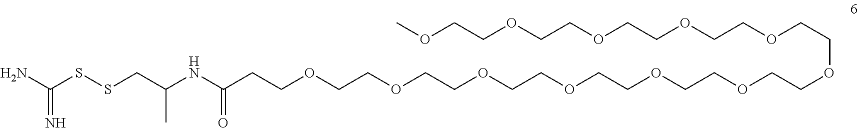 [SMILES: COCCOCCOCCOCCOCCOCCOCCOCCOCCOCCOCCOCCC(=O)NC(C)CSSC(=N)N]

Compound 6 was prepared using similar procedures as those used for compound 1, replacing cystamine with 2-aminopropane-1-thiol. Compound 6 was isolated using preparative HPLC (Method 3) (4 mg, 18% yield). Analytical UPLC-MS (Method 1): Retention time=0.97 min, m/z (ES+) (M+H)⁺: 736.37 (theoretical); 736.37 (observed).

### Synthesis of 38-oxo-2,5,8,11,14,17,20,23,26,29,32,35-dodecaoxa-39-azatetratetracontan-44-yl carbamo(dithioperoxo)imidate (7)

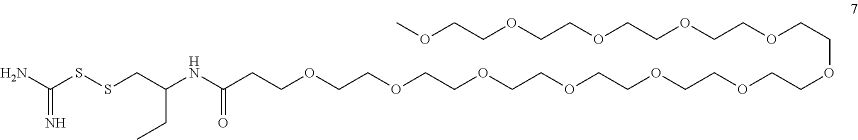 [SMILES: CCC(CSSC(=N)N)NC(=O)CCOCCOCCOCCOCCOCCOCCOCCOCCOCCOCCOCCOC]

Compound 7 was prepared using similar procedures as those used for compound 1, replacing cystamine with 2-aminobutane-1-thiol. Compound 7 was isolated using preparative HPLC (Method 3) (7 mg, 31% yield). Analytical UPLC-MS (Method 1): Retention time=1.03 min, m/z (ES+) (M+H)⁺: 750.38 (theoretical); 750.32 (observed).

### Synthesis of S-(carbamimidoylthio)-N-(2,5,8,11,14,17,20,23,26,29,32,35-dodecaoxaoctatriacontan-38-oyl)-L-cysteine (8)

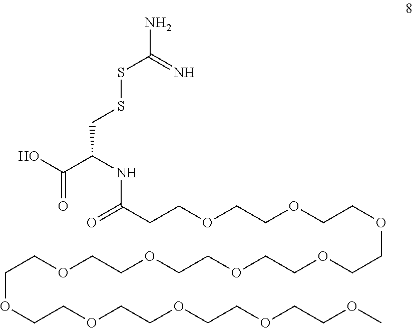 [SMILES: COCCOCCOCCOCCOCCOCCOCCOCCOCCOCCOCCOCCC(=O)N[C@@H](CSSC(=N)N)C(=O)O]

Compound 8 was prepared using similar procedures as those used for compound 1, replacing cystamine with L-cysteine. Compound 8 was isolated using preparative HPLC (Method 3) (2 mg, 9% yield). Analytical UPLC-MS (Method 1): Retention time=1.02 min, m/z (ES+) (M+H)⁺: 766.34 (theoretical); 766.64 (observed).

### Example 2

### Synthesis of Cleavable Moieties with Maleimide Groups

### Synthesis of 3-(2,5-dioxo-2,5-dihydro-1H-pyrrol-1-yl)-N-(2,5,8,11,14,17,20,23,26,29,32,35-dodecaoxaheptatriacontan-37-yl)propanamide (9)

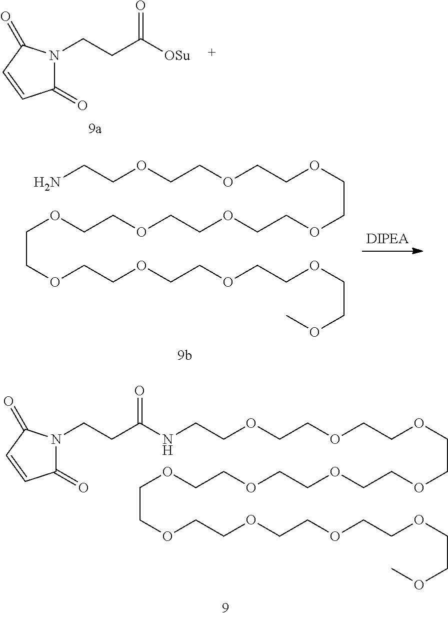

A 4-mL glass vial equipped with a stir bar was charged with maleimidopropionic OSu ester (9a, 3.0 mg, 0.011 mmol), PEG12 amine (9b, 6.3 mg, 0.011 mmol), DIPEA (3.9 μL, 0.023 mmol) and dichloromethane (DCM, 300 μL). The reaction mixture was stirred at RT for 4 h. The reaction mixture was then concentrated in vacuo, and the resulting residue re-dissolved in DMSO (500 μL). The reaction mixture was loaded onto a preparative HPLC and compound 9 was isolated using Method 3 (5 mg, 62% yield). Analytical UPLC-MS (Method 1): Retention time=1.20 min, m/z (ES+) (M+H)⁺: 711.39 (theoretical); 711.22 (observed).

### Synthesis of 3-(2,5-dioxo-2,5-dihydro-1H-pyrrol-1-yl)-N-(2,5,8,11,14,17,20,23,26,29,32,35,38,41,44,47,50,53,56,59,62,65,68,71,74,77,80,83,86,89,92,95,98,101,104,107,110,113,116,119,122,125,128,131,134,137,140,143-oetatetracontaoxapentatetracontahectan-145-yl)propanamide (10)

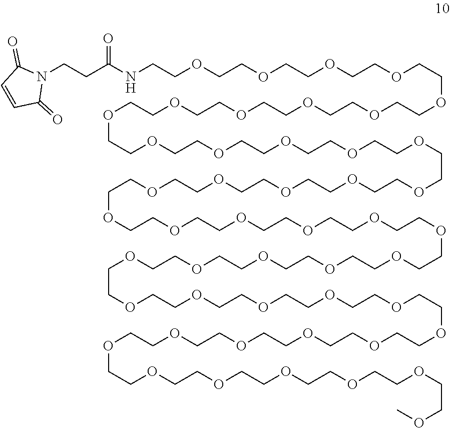 [SMILES: COCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCNC(=O)CCN1C(=O)C=CC1=O]

Compound 10 was prepared using similar procedures as those used for compound 9, replacing PEG12 amine (9b) with PEG48 amine. Compound 10 was isolated using preparative HPLC (Method 4) (8 mg, 31% yield). Analytical UPLC-MS (Method 1): Retention time=1.46 min, m/z (ES+) (M+2H)²⁺: 1149.17 (theoretical); 1149.67 (observed).

### Synthesis of 6-(2,5-dioro-2,5-dihydro-1H-pyrrol-1-yl)-N-(2,5,8,11-tetraoxatridecan-13-yl)hexanamide (11)

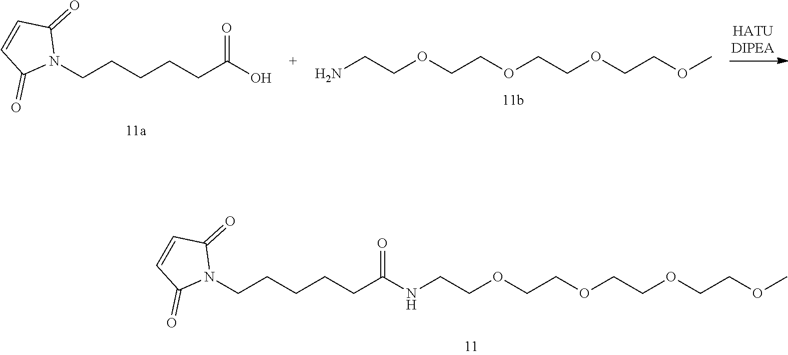 [SMILES: O=C(O)CCCCCN1C(=O)C=CC1=O ; COCCOCCOCCOCCN ; COCCOCCOCCOCCNC(=O)CCCCCN1C(=O)C=CC1=O]

A 4-mL glass vial equipped with a stir bar was charged with 6-maleimidocaproic acid (11a, 20.4 mg, 0.096 mmol), 1-[bis(dimethylamino)methylene]-1H-1,2,3-triazolo[4,5-b]pyridinium 3-oxid hexafluorophosphate (HATU, 34.9 mg, 0.092 mmol), anhydrous DMF (0.5 mL), and DIPEA (0.050 mL, 0.289 mmol). The mixture was stirred at RT for 20 min. Amino-PEG4 (11b, 20 mg, 0.096 mmol) was added to the vial and the resulting mixture was stirred at RT for 3 h. The solvent was removed in vacuo and the residue was taken up in 0.1% (v/v) aqueous TFA. The reaction mixture was loaded onto a preparative HPLC (Method 4) and the fractions containing compound 11 were combined and lyophilized to yield compound 11 (27% yield). Analytical UPLC-MS (Method 1): Retention time=1.64 min, m/z (ES+) (M+H)⁺: 401.22 (theoretical); 401.20 (observed).

### Synthesis of 6-(2,5-dioxo-2,5-dihydro-1H-pyrrol-1-yl)-N-(2,5,8,11,14,17,20,23,26,29,32,35-dodecaoxaheptatriacontan-37-yl)hexanamide (12)

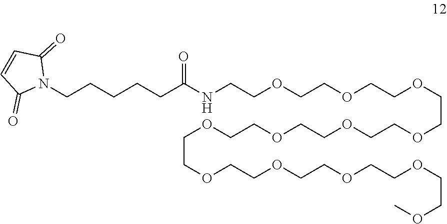 [SMILES: COCCOCCOCCOCCOCCOCCOCCOCCOCCOCCOCCOCCNC(=O)CCCCCN1C(=O)C=CC1=O]

Compound 12 was prepared using similar procedures as those used for compound 11, replacing amino PEG4 (11b) with amino PEG12 (9b). Compound 12 was isolated using preparative HPLC (Method 4) (30% yield). Analytical UPLC-MS (Method 1): Retention time=1.79 min, m/z (ES+) (M+H)⁺: 753.43 (theoretical); 753.42 (observed).

### Synthesis of 6-(2,5-dioxo-2,5-dihydro-1H-pyrrol-1-yl)-N-(2, 5,8, 11, 14, 17,20,23,26,29,32,35, 38,41,44,47,50,53,56,59,62,65,68,71,74,77,80,83,86,89,92,95,98, 101,104, 107,110, 113,116, 119, 122, 125,128, 131,134, 137,140, 143-octatetracontaoxapentatetracontahectan-145-yl)hexanamide (13)

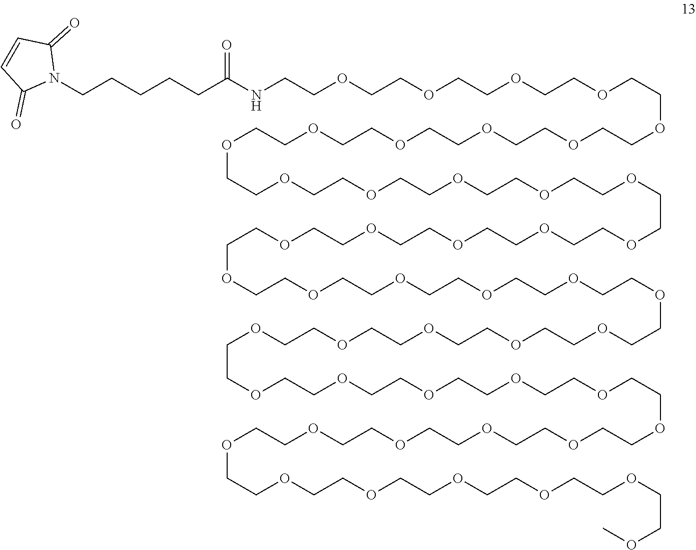 [SMILES: COCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCNC(=O)CCCCCN1C(=O)C=CC1=O]

Compound 13 was prepared using similar procedures as those used for compound 11, replacing amino PEG4 (11b) with amino PEG48. Compound 13 was isolated using preparative HPLC (Method 4) (18% yield). Analytical UPLC-MS (Method 1): Retention time=2.05 min, m/z (ES+) (M+2H)²⁺: 1170.20 (theoretical); 1170.18 (observed).

### Synthesis of 6-(2,5-dioxo-2,5-dihydro-1H-pyrrol-1-yl)-N-(2,5,8,11,14,17,20,23-octaoxapentacosan-25-yl)hexanamide (14)

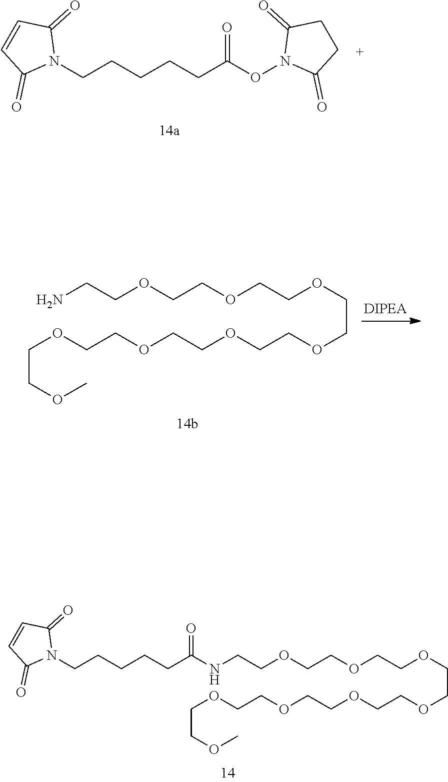 [SMILES: O=C(CCCCCN1C(=O)C=CC1=O)ON1C(=O)CCC1=O ; COCCOCCOCCOCCOCCOCCOCCOCCN ; COCCOCCOCCOCCOCCOCCOCCOCCNC(=O)CCCCCN1C(=O)C=CC1=O]

A 4-mL glass vial equipped with a stir bar was charged with 2,5-dioxopyrrolidin-1-yl 6-(2,5-dioxo-2,5-dihydro-1H-pyrrol-1-yl)hexanoate (14a, 19.29 mg, 0.063 mmol), amino-PEG8 (14b, 20 mg, 0.052 mmol), anhydrous DMF (0.5 mL), and DIPEA (0.045 mL, 0.261 mmol). The mixture was stirred at RT for 3 h. The solvent was then removed in vacuo and the resulting residue was taken up in 0.1% (v/v) aqueous TFA. The reaction mixture was loaded onto a preparative HPLC (Method 4) and the fractions containing compound 14 were combined and lyophilized to yield compound 14 (17.73 mg, 58.95% yield). Analytical UPLC-MS (Method 1): Retention time=1.69 min, m/z (ES+) (M+H)⁺: 577.33 (theoretical); 577.28 (observed).

### Synthesis of 6-(2,5-dioxo-2,5-dihydro-1H-pyrrol-1-yl)-N-(2,5,8,11,14,17,20,23,26,29,32,35,38,41,44,47,50,53,56,59,62,65,68,71-tetracosaoxaniheptacontan-73-yl)hexanamide (15)

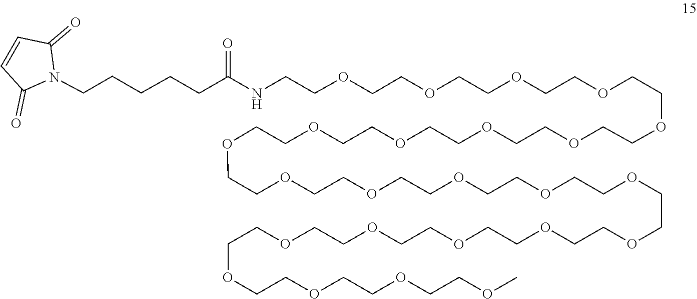 [SMILES: COCCOCCOCCOCCOCCOCCOCCOCCOCCOCCOCCOCCOCCOCCOCCOCCOCCOCCOCCOCCOCCOCCOCCOCCNC(=O)CCCCCN1C(=O)C=CC1=O]

Compound 15 was prepared using similar procedures as those used for compound 14, replacing amino PEG8 (14b) with amino PEG24. Compound 15 was isolated using preparative HPLC (Method 4) (19.48 mg, 82.72% yield). Analytical UPLC-MS (Method 1): Retention time=1.87 min, m/z (ES+) (M+H)⁺: 1281.75 (theoretical); 1281.72 (observed).

### Synthesis of 6-(2,5-dioxo-2,5-dihydro-1H-pyrrol-1-yl)-N-(2,5,8,11,14,17,20,23,26,29,32,35,38,41,44,47,50,53,56,59,62,65,68,71,74,77,80,83,86,89,92,95,98,101,104,107-hexatriacontaoxanonahectan-109-yl)hexanamide (16)

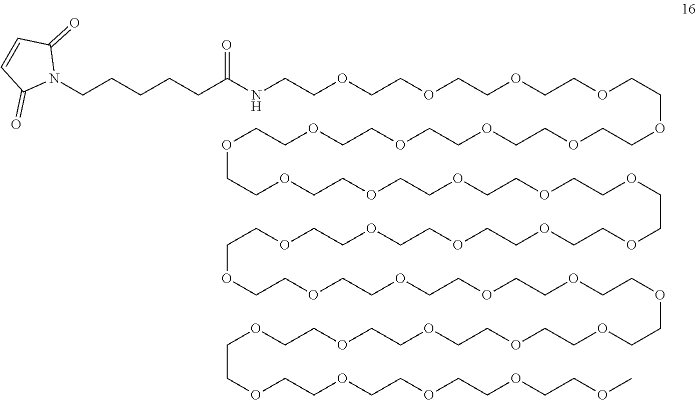 [SMILES: COCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCOCCNC(=O)CCCCCN1C(=O)C=CC1=O]

Compound 16 was prepared using similar procedures as those used for compound 14, replacing amino PEG8 (14b) with amino PEG36. Compound 16 was isolated using preparative HPLC (Method 4) (16.76 mg, 74.86% yield). Analytical UPLC-MS (Method 1): Retention time=1.93 min, m/z (ES+) (M+H)⁺: 1810.06 (theoretical); 1810.02 (observed).

### Synthesis of 6-(2,5-dioxo-2,5-dihydro-1H-pyrrol-1-yl)-N,N-di(2,5,8,11-tetraoxatridecan-13-yl)hexanamide (17)

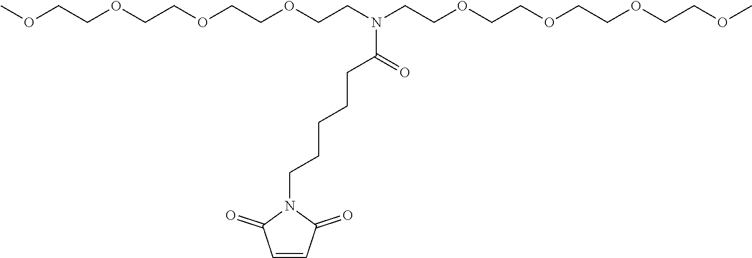

Compound 17 was prepared using similar procedures as those used for compound 14, replacing amino PEG8 (14b) with di(2,5,8,11-tetraoxatridecan-13-yl)amine. Compound 17 was isolated using preparative HPLC (Method 4) (19.11 mg, 64.30% yield). Analytical UPLC-MS (Method 1): Retention time=1.84 min, m/z (ES+) (M+H)⁺: 591.34 (theoretical); 591.31 (observed).

### Synthesis of 1-(2,5-dioro-2,5-dihydro-1H-pyrrol-1-yl)-3-oxo-7,10,13,16-tetraoxa-4-azanonadecan-19-oic acid (18)

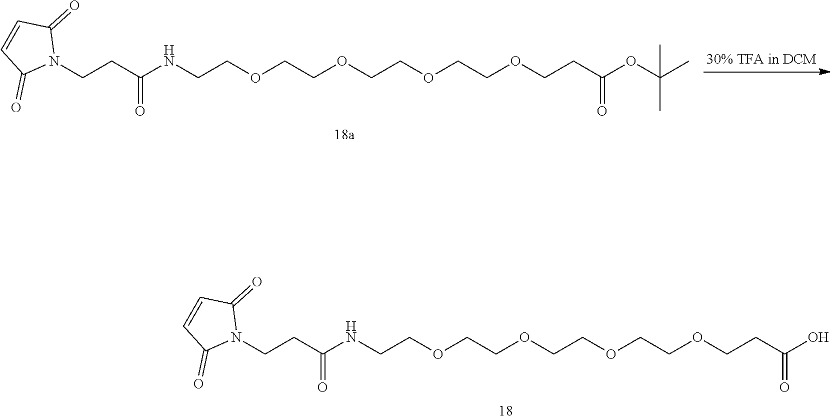 [SMILES: CC(C)(C)OC(=O)CCOCCOCCOCCOCCNC(=O)CCN1C(=O)C=CC1=O ; O=C(O)CCOCCOCCOCCOCCNC(=O)CCN1C(=O)C=CC1=O]

A 4-mL glass vial equipped with a stir bar was charged with mp-PEG4-OtBu (18a, 22 mg, 0.047 mmol) and 30% (v/v) TFA in DCM (1 mL). The mixture was stirred at RT for 1 h. The solvent was then removed in vacuo and the resulting residue was taken up in 0.1% (v/v) aqueous TFA. The reaction mixture was loaded onto a preparative HPLC system (Method 4) and the fractions containing compound 18 were combined and lyophilized to yield compound 18 (18.97 mg, 98.08% yield). Analytical UPLC-MS (Method 1): Retention time=1.39 min, m/z (ES+) 417.18 (M+H)⁺: 417.18 (theoretical); 417.15 (observed).

### Synthesis of 3,3′-((2-(22-(2,5-dioxo-2,5-dihydro-1H-pyrrol-1-yl)-17-oxo-4,7,10,13-tetraoxa-16-azadocosanamido)-2-(27-oxo-2,5,8,11,14,17,20,23,30-nonaoxa-26-azahentriacontan-31-yl)propane-1,3-diyl)bis(oxy))bis(N-(2,5,8,11,14,17,20,23-octaompentacosan-25-yl)propanamide) (19)

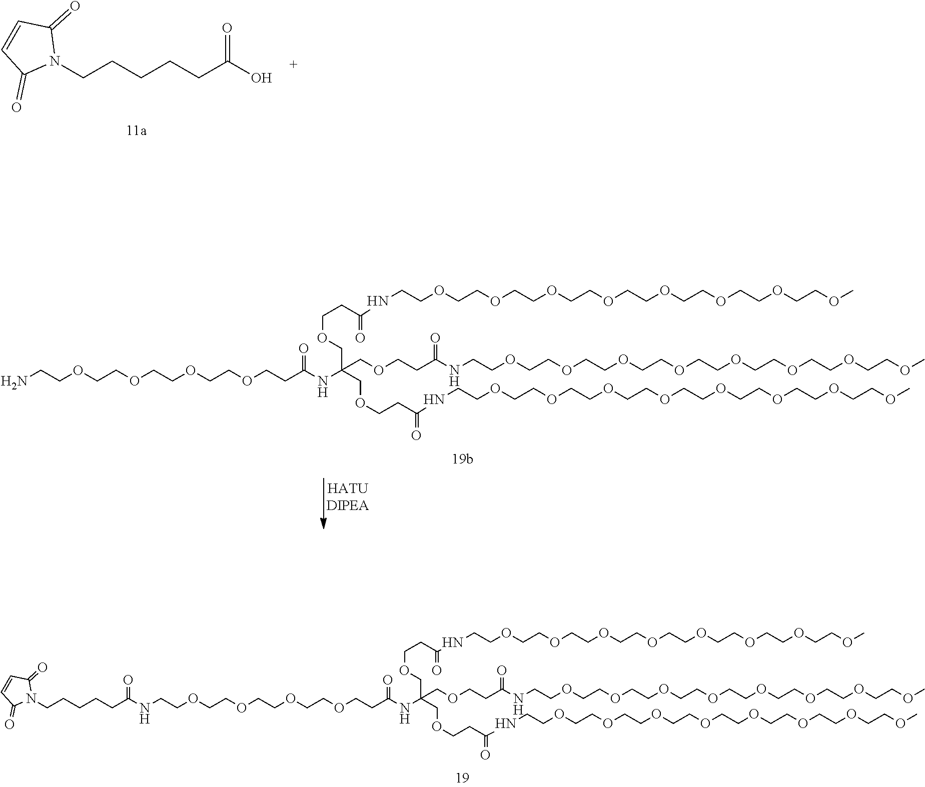 [SMILES: O=C(O)CCCCCN1C(=O)C=CC1=O ; COCCOCCOCCOCCOCCOCCOCCOCCNC(=O)CCOCC(COCCC(=O)NCCOCCOCCOCCOCCOCCOCCOCCOC)(COCCC(=O)NCCOCCOCCOCCOCCOCCOCCOCCOC)NC(=O)CCOCCOCCOCCOCCN ; COCCOCCOCCOCCOCCOCCOCCOCCNC(=O)CCOCC(COCCC(=O)NCCOCCOCCOCCOCCOCCOCCOCCOC)(COCCC(=O)NCCOCCOCCOCCOCCOCCOCCOCCOC)NC(=O)CCOCCOCCOCCOCCNC(=O)CCCCCN1C(=O)C=CC1=O]

A 4-mL glass vial equipped with a stir bar was charged with 6-maleimidocaproic acid (11a, 1.67 mg, 0.008 mmol), HATU (2.86 mg, 0.008 mmol), anhydrous DMF (0.5 mL), and DIPEA (0.004 mL, 0.024 mmol). The mixture was stirred at RT for 20 min. Amino-PEG4-(PEG8)₃ (19b, 30 mg, 0.008 mmol) was added to the vial and the mixture was stirred at RT for 3 h. The solvent was then removed in vacuo and the resulting residue was taken up in 0.1% (v/v) aqueous TFA. The reaction was loaded onto a preparative HPLC system (Method 4) and the fractions containing compound 19 were combined and lyophilized to yield compound 19 (25% yield). Analytical UPLC-MS (Method 1): Retention time=1.91 min, m/z (ES+) (M+H)⁺: 1874.08 (theoretical); 1874.04 (observed).

### Synthesis of 3,3′-((2-(22-(2,5-dioxo-2,5-dihydro-1H-pyrrol-1-yl)-17-oxo-4,7,10,13-tetraoxa-16-azadocosanannclo)-2-(75-oxo-2,5,8,11,14,17,20,23,26,29,32,35,38,41,44,47,50,53,56,59,62,65,68,71,78-pentacosaoxa-74-azanonaheptacontan-79-yl)propane-1,3-diyl)bis(oxy))bis(N-(2,5,8,11,14,17,20,23,26,29,32,35,38,41,44,47,50,53,56,59,62,65,68,71-tetracosaoxatriheptacontan-73-yl)propanamide) (20)

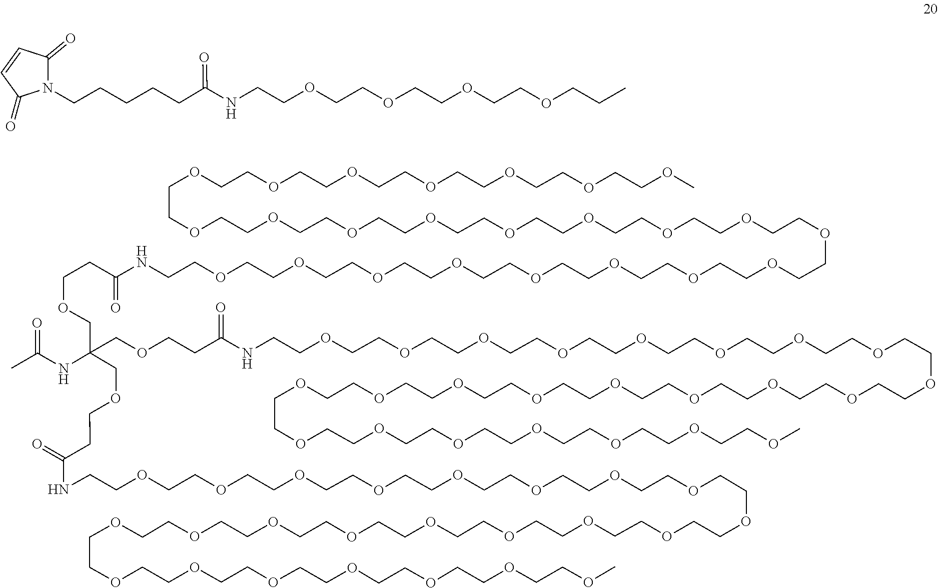

Compound 20 was prepared using similar procedures as those used for compound 19, replacing amino-PEG4-(PEG8)₃ (19b) with amino-PEG4-(PEG24)₃. Compound 20 was isolated using preparative HPLC (Method 4) (21% yield). Analytical UPLC-MS (Method 1): Retention time=1.99 min, m/z (ES+) (M+2H)²⁺: 1995.18 (theoretical); 1995.11 (observed).

### Synthesis of 7-(2,5-dioxo-2,5-dihydro-1H-pyrrol-1-yl)-N-(2,5,8,11,14,17,20,23,26,29,32,35-dodecaoxahepianiacontan-37-yl)heptanantide (21)

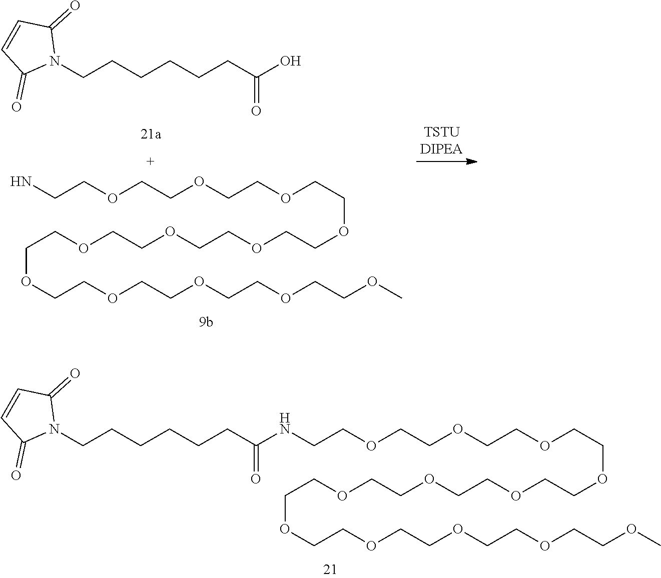 [SMILES: O=C(O)CCCCCCN1C(=O)C=CC1=O ; COCCOCCOCCOCCOCCOCCOCCOCCOCCOCCOCCOCCN ; COCCOCCOCCOCCOCCOCCOCCOCCOCCOCCOCCOCCNC(=O)CCCCCCN1C(=O)C=CC1=O]

A 4-mL glass vial equipped with a stir bar was charged with 7-maleimidoheptanoic acid (21a, 20.4 mg, 0.096 mmol), O—(N-Succinimidyl)-N,N,N′,N′-tetramethyluronium tetrafluoroborate (TSTU, 34.9 mg, 0.092 mmol), anhydrous DMF (0.5 mL), and DIPEA (0.050 mL, 0.289 mmol). The mixture was stirred at RT for 20 min. Amino-PEG12 (9b, 20 mg, 0.096 mmol) was added to the vial and the mixture was stirred at RT for 3 h. The solvent was then removed in vacuo and the resulting residue was taken up in 0.1% (v/v) aqueous TFA. The reaction mixture was loaded onto a preparative HPLC system (Method 4) and the fractions containing compound 21 were combined and lyophilized to yield compound 21 (50% yield). Analytical UPLC-MS (Method 2): Retention time=1.39 min, m/z (ES+) (M+Na)⁺: 790.44 (theoretical); 789.93 (observed).

### Synthesis of 8-(2,5-dioxo-2,5-dihydro-111-pyrrol-1-yl)-N-(2,5,8,11,14,17,20,23,26,29,32,35-dodecaoxaheptatriacontan-37-yl)octanamide (22)

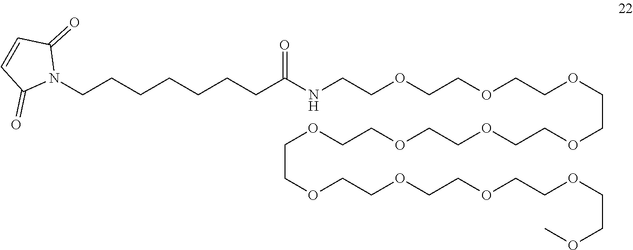 [SMILES: COCCOCCOCCOCCOCCOCCOCCOCCOCCOCCOCCOCCNC(=O)CCCCCCCN1C(=O)C=CC1=O]

### Synthesis of 8-(2,5-dioxo-2,5-dihydro-11-1-pyrrol-1-yl)octanoic acid (22c)

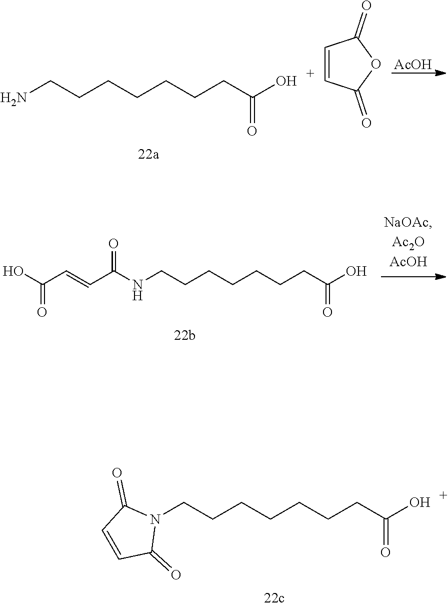

A 20-mL glass vial equipped with a stir bar was charged with 8-aminooctanoic acid (22a, 66.58 mg, 0.418 mmol), maleic anhydride (40 mg, 0.418 mmol), and glacial acetic acid (AcOH, 5 mL). The mixture was stirred at 40° C. for 1 h. The solvent was then removed in vacuo and the residue was taken up in DCM (4 mL). Compound 22b was obtained by precipitation using cold hexanes and isolated by filtration (85.1 mg, 79.11% yield) and used in subsequent steps without further purification. Analytical UPLC-MS (Method 2): Retention time=1.30 min, m/z (ES−) (M−H)⁻: 256.13 (theoretical); 256.26 (observed).

A 4-mL glass vial equipped with a stir bar was charged with compound 22b (10 mg, 0.039 mmol), sodium acetate (NaOAc, 1.59 mg, 0.019 mmol), glacial acetic acid (AcOH, 0.002 mL, 0.039 mmol), and acetic anhydride (Ac₂O, 1 mL). The mixture was stirred at 60° C. for 1 h. The solvent was then removed in vacuo and the resulting residue was resuspended in and azeotroped with toluene (3×2 mL) to yield compound 22c (8.02 mg, 86.24% yield), which was used in subsequent steps without further purification. Analytical UPLC-MS (Method 2): Retention time=1.91 min, m/z (ES+) (M+H)⁺: 240.12 (theoretical); 240.17 (observed).

### Synthesis of dodecaoxaheptatriacontan-37-yl)octanamide (22)

Compound 22 was prepared using similar procedures as those used for compound 21, replacing 7-maleimidoheptanoic acid (21a) with 8-(2,5-dioxo-2,5-dihydro-1H-pyrrol-1-yl)octanoic acid (22c). Compound 22 was isolated using preparative HPLC (Method 4) (4.73 mg, 14.55% yield). Analytical UPLC-MS (Method 2): Retention time=1.50 min, m/z (ES+) (M+H)⁺: 781.47 (theoretical); 781.92 (observed).

### Synthesis of 8-(2,5-dioxo-2,5-dihydro-1H-pyrrol-1-yl)-N-(2,5,8,11,14,17,20,23,26,29,32,35-dodecaoxaheptatriacontan-37-yl)octanamide (23)

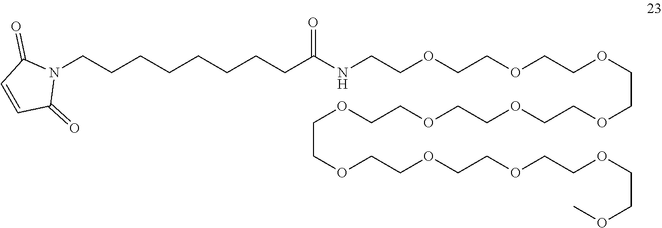 [SMILES: COCCOCCOCCOCCOCCOCCOCCOCCOCCOCCOCCOCCNC(=O)CCCCCCCCN1C(=O)C=CC1=O]

### Synthesis of 8-(2,5-dioxo-2,5-dihydro-1H-pyrrol-1-yl)octanoic acid (23d)

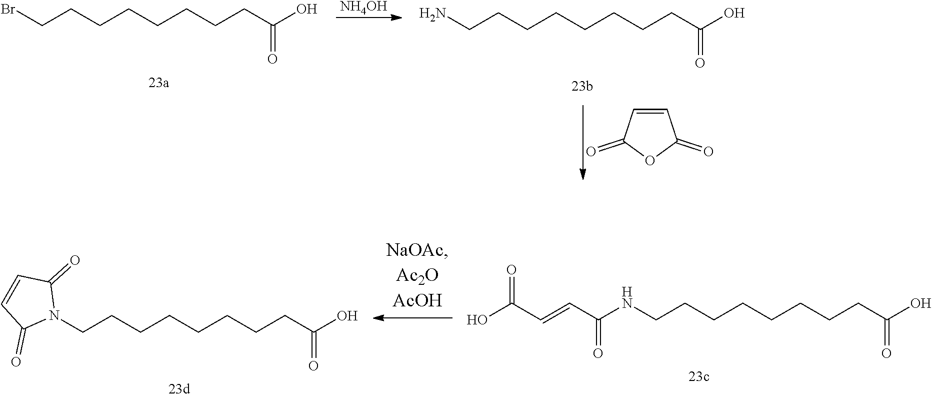

A 20-mL glass vial equipped with a stir bar was charged with bromonanoic acid (23a, 30 mg, 0.127 mmol), aqueous ammonium hydroxide (1.5 mL), and DMF (5 mL). The mixture was stirred at RT overnight. The solvent was then removed in vacuo and the resulting residue was suspended in and azeotroped with toluene (3×3 mL) to yield compound 23b which was used in subsequent steps without further purification. Analytical UPLC-MS (Method 2): Retention time=0.81 min, m/z (ES+) (M+H)⁺: 174.14 (theoretical); 174.13 (observed).

A 20-mL glass vial equipped with a stir bar was charged with the crude 9-aminonanoic acid (23b, 31.1 mg, 0.180 mmol), maleic anhydride (17.60 mg, 0.180 mmol), and glacial acetic acid (5 mL). The mixture was stirred at 40° C. for 1 h. The solvent was then removed in vacuo and the resulting residue was taken up in DCM (4 mL). Compound 23c was obtained by precipitation using cold hexanes and isolated by filtration. The crude 23c isolated was used in subsequent steps without further purification. Analytical UPLC-MS (Method 2): Retention time=1.41 min, m/z (ES+) (M+H)⁺: 272.15; 272.20 (observed).

A 4-mL glass vial equipped with a stir bar was charged with crude nanoic acid (23c, 10 mg, 0.039 mmol), sodium acetate (1.51 mg, 0.018 mmol), glacial acetic acid (2 μL, 0.037 mmol), and acetic anhydride (1 mL). The mixture was stirred at 60° C. for 1 h. The solvent was then removed in vacuo and the resulting residue was resuspended in and azeotroped with toluene (3×2 mL) to yield compound 23d, which was used in subsequent steps without further purification. Analytical UPLC-MS (Method 2): Retention time=1.77 min, m/z (ES+) (M+H)⁺: 254.13; 254.00 (observed).

### Synthesis of 8-(2,5-dioxo-2,5-dihydro-1H-pyrrol-1-yl)-N-(2,5,8,11,14,17,20,23,26,29,32,35-dodecaoxaheptatriacontan-37-yl)octanamide (23)

Compound 23 was prepared using similar procedures as those used for compound 21, replacing 7-maleimidoheptanoic acid (21a) with 8-(2,5-dioxo-2,5-dihydro-1H-pyrrol-1-yl)octanoic acid (23d). Compound 23 was isolated using preparative HPLC (Method 3) (0.80 mg, 2.7% yield). Analytical UPLC-MS (Method 2): Retention time=1.60 min, m/z (ES+) (M+H)⁺: 795.48 (theoretical); 796.04 (observed).

### Synthesis of 4-((2,5-dioxo-2,5-dihydro-1H-pyrrol-1-yl)diphenylmethyl)-N-(2, 5, 8, 11, 14, 17, 20,23, 26,29,32,35-dodecaoxaheptatriacontan-37-yl)benzamide (24)

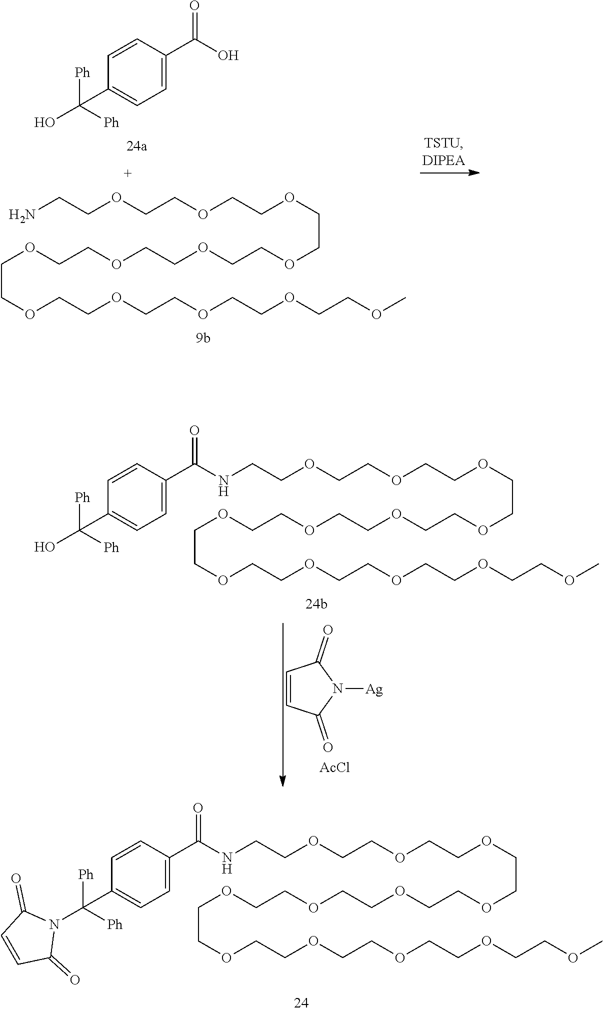

A 4-mL vial equipped with a stir bar was charged with 4-(hydroxydiphenylmethyl)benzoic acid (24a, 100 mg, 0.33 mmol), TSTU (100 mg, 0.33 mmol), DIPEA (0.17 μL, 0.99 mmol), and DMF (2 mL). The reaction was stirred for 30 min at RT, then amino-PEG12 (9b, 183 mg, 0.33 mmol) was added. The reaction mixture was stirred for 3 h and concentrated in vacuo. Compound 24b (100 mg, 36% yield) was isolated by preparative HPLC (Method 4). Analytical UPLC-MS (Method 1): Retention time=1.81 min, m/z (ES+) (M+H)⁺: 846.46 (theoretical); 846.33 (observed).

A 4-mL glass vial equipped with a stir bar was charged with compound 24b (100 mg, 0.12 mmol) and DCM (1 mL). Acetyl chloride (AcCl, 16.5 μL, 0.23 mmol) was added to the reaction mixture at RT and the mixture was stirred for 2 h. Silver maleimide (52 mg, 0.25 mmol) was added and the reaction mixture was stirred at RT for 4 h, upon which the reaction mixture was filtered and concentrated in vacuo. Compound 24 (26 mg, 24% yield) was isolated by preparative HPLC (Method 4). Analytical UPLC-MS (Method 1): Retention time=1.84 min, m/z (ES+) (M+Na)⁺: 925.47 (theoretical); 925.99 (observed).

### Synthesis of (E)-N-(2, 5, 8,11, 14, 17,20, 23,26, 29, 32, 35-dodecaoxaheptatriacontan-37-yl)-4-(2-nitrovinyl)-3-(trifluoromethyl)benzamide (25)

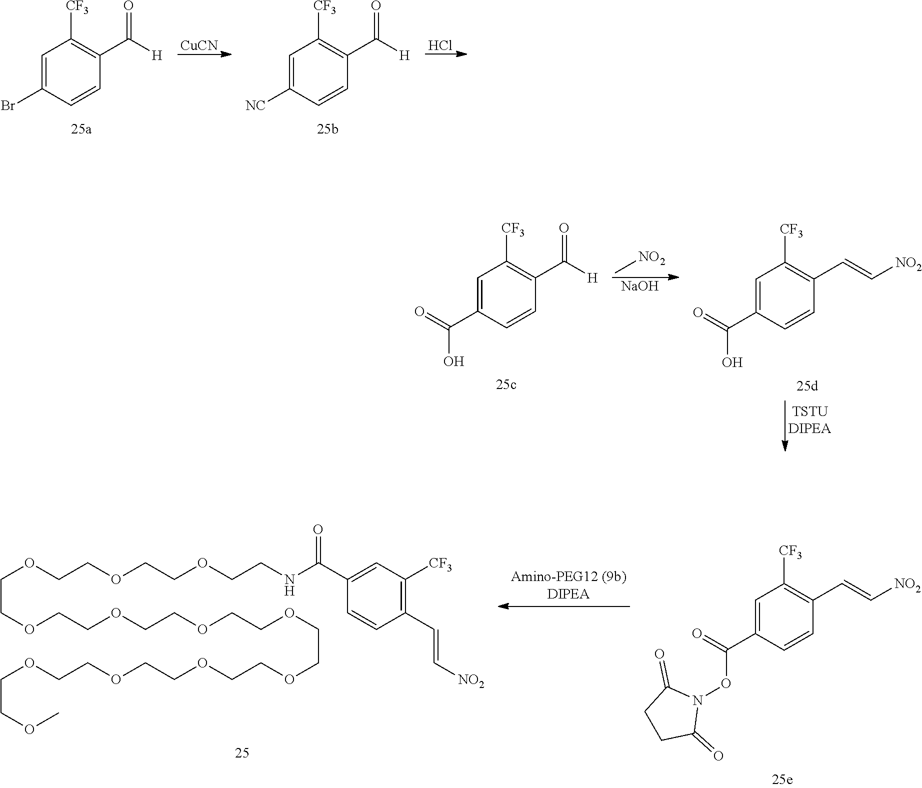

### Synthesis of 4-formyl-3-(trifluoromethyl)benzonitrile (25b)

A 5-mL microwave-compatible vial equipped with a stir bar was charged with 4-bromo-2-(trifluoromethyl)benzaldehyde (25a, 100 mg, 0.395 mmol), copper (I) cyanide (46.02 mg, 0.514 mmol), and N-methyl-2-pyrrolidone (NMP, 4 mL). The mixture was heated to 200° C. and stirred for 15 min in a microwave reactor. The reaction mixture was then diluted with DCM (20 mL) and filtered through celite. The solvent was removed in vacuo and compound 25b (62.47 mg, 79.38% yield) was isolated by flash column chromatography using a silica gel column and elution using a gradient of 0-20% ethyl acetate in hexanes.

### Synthesis of 4-formyl-3-(trifluoromethyl)benzoic acid (25c)

A 20-mL glass vial equipped with a stir bar was charged with compound 25b (62.47 mg, 0.314 mmol), and concentrated HCl (7 mL). The mixture was heated to 90° C. and stirred for 1 h. The reaction mixture was then diluted with water (30 mL) and extracted with 3×30 mL ethyl acetate (EtOAc). The organic layers were combined and washed with 3×30 mL brine, dried over magnesium sulfate (MgSO₄) and the solvent was removed in vacuo to yield compound 25c (52.80 mg, 77.16% yield), which was used in subsequent steps without further purification. Analytical UPLC-MS (Method 2): Retention time=1.63 min, m/z (ES−) (M−H)⁻: 217.02 (theoretical); 217.03 (observed).

### Synthesis of (E)-4-(2-nitrovinyl)-3-(trifluoromethyl)benzoic acid (25d)

A 20-mL glass vial equipped with a stir bar was charged with compound 25c, nitromethane (0.064 mL, 1.174 mmol), 1M aqueous sodium hydroxide (0.082 mL, 0.367 mmol) and methanol (MeOH, 5 mL). The mixture was stirred at RT for 2 h. The reaction was quenched by adding 6M aqueous HCl (0.014 mL) and stirred at 0° C. for 15 min. The reaction mixture was extracted with DCM (3×15 mL), and the organic layers were combined and washed with brine (3×15 mL), and dried over MgSO₄ and the solvent was removed in vacuo to yield compound 25d (7.28 mg, 19% yield), which was isolated by flash column chromatography using a silica gel column and elution using a gradient of 30-100% EtOAc in hexanes, followed by elution using a gradient of 0-40% MeOH in DCM. Analytical UPLC-MS (Method 2): Retention time=1.78 min, m/z (ES−) (M−H)⁻: 260.02 (theoretical); 260.04 (observed).

### Synthesis of (E)-N-(2, 5, 8, 11, 14, 17,20, 23, 26, 29,32, 35-dodecaoxaheptatriacontan-37-yl)-4-(2-nitrovinyl)-3-(trifluoromethyl)benzamide (25)

A 4-mL vial equipped with a stir bar was charged with compound 25d (2.27 mg, 0.009 mmol), TSTU (2.24 mg, 0.010 mmol), DIPEA (0.005 mL, 0.026 mmol) and anhydrous DMF (0.5 mL). The mixture was stirred at RT for 1 h, and the solvent was removed in vacuo to yield compound 25e, which was not isolated. Amino-PEG12 (9b, 5.63 mg, 0.010 mmol), DIPEA (0.004 mL, 0.025 mmol) and anhydrous DMF (0.5 mL) were added to the crude 25e, and the mixture was stirred at RT for 2 h. The solvent was removed in vacuo and the crude product was purified by HPLC (Method 3) to yield compound 25 (1.69 mg, 25.1% yield). Analytical UPLC-MS (Method 2): Retention=1.75 min, m/z (ES−) (M−H)⁻: 801.37 (theoretical); 801.43 (observed).

### Synthesis of (Z)-6-(2-(3-(2,5-dioxo-2,5-dihydro-1H-pyrrol-1-yl)propanoyl)hydrazono)-N-(2,5,8,11,14,17,20,23,26,29,32,35-dodecaoxaheptatriacontan-37-yl)-6-phenylhexanamide (26)

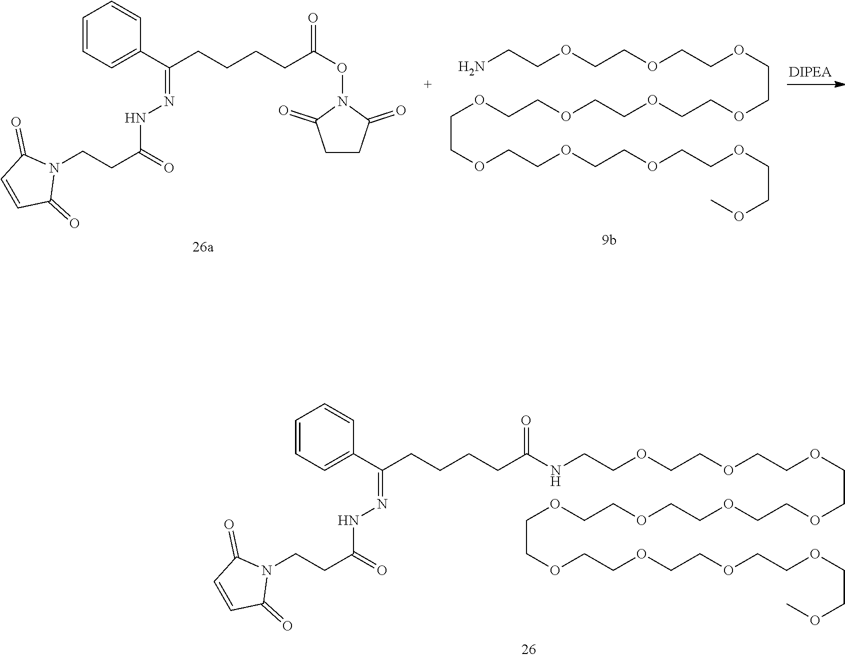 [SMILES: O=C(CCN1C(=O)C=CC1=O)N/N=C(/CCCCC(=O)ON1C(=O)CCC1=O)c1ccccc1 ; COCCOCCOCCOCCOCCOCCOCCOCCOCCOCCOCCOCCN ; COCCOCCOCCOCCOCCOCCOCCOCCOCCOCCOCCOCCNC(=O)CCCC/C(=N/NC(=O)CCN1C(=O)C=CC1=O)c1ccccc1]

A 4-mL glass vial equipped with a stir bar was charged with 2,5-dioxopyrrolidin-1-yl(Z)-6-(2-(3-(2,5-dioxo-2,5-dihydro-1H-pyrrol-1-yl)propanoyl)hydrazono)-6 phenylhexanoate (26a, 10 mg, 0.021 mmol), amino-PEG12 (9b, 9.96 mg, 0.018 mmol), anhydrous DMF (1.0 mL), and DIPEA (0.009 mL, 0.053 mmol). The mixture was stirred at RT for 3 h. The solvent was then removed in vacuo and the resulting residue was taken up in MeOH (2 mL). Compound 26 (9.46 mg, 58.24% yield) was isolated by flash column chromatography using a silica gel column and elution using a gradient of 0-25% MeOH in DCM. Analytical UPLC-MS (Method 2): Retention time=1.60 min, m/z (ES+) (M+H)⁺: 913.50 (theoretical); 913.76 (observed).

### Synthesis of (E)-4-(1-(2-(3-(2,5-dioxo-2,5-dihydro-1yl)pyrrol-1-propanoyl)hydrazono)ethyl)-N-(2,5,8,11,14,17,20,23,26,29,32,35-dodecaoxaheptatriacontan-37yl)benzamide (27)

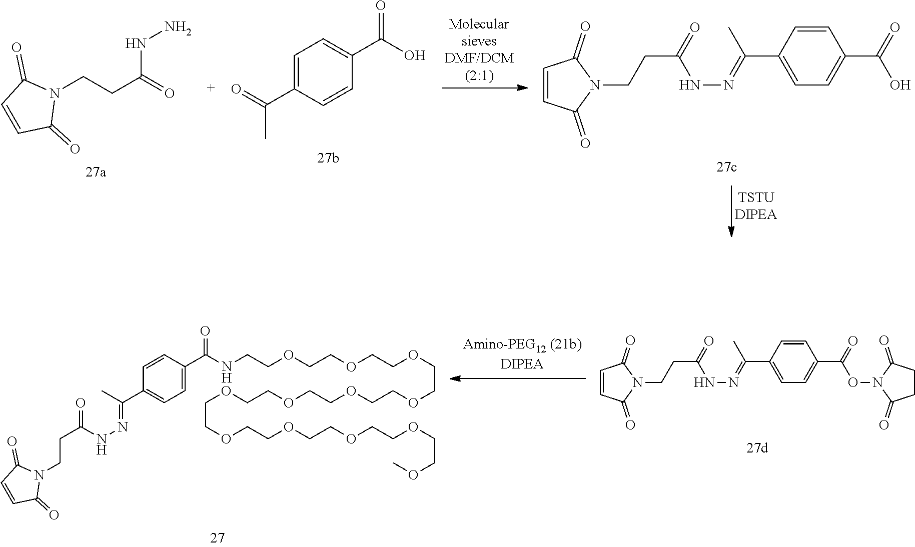 [SMILES: NNC(=O)CCN1C(=O)C=CC1=O ; CC(=O)c1ccc(C(=O)O)cc1 ; C/C(=N\NC(=O)CCN1C(=O)C=CC1=O)c1ccc(C(=O)O)cc1 ; COCCOCCOCCOCCOCCOCCOCCOCCOCCOCCOCCOCCNC(=O)c1ccc(/C(C)=N/NC(=O)CCN2C(=O)C=CC2=O)cc1 ; C/C(=N\NC(=O)CCN1C(=O)C=CC1=O)c1ccc(C(=O)ON2C(=O)CCC2=O)cc1]

A 4-mL glass vial equipped with a stir bar was charged with 3-maleimido-propionic acid hydrazide (27a, 10 mg, 0.055 mmol), 4-acetylbenzoic acid (27b, 8.96 mg, 0.055 mmol), molecular sieves, and 2:1 DMF/DCM (1.0 mL). The mixture was stirred at RT for 3 h. The reaction mixture was then filtered, and the solvent was removed in vacuo to yield crude compound 27c, which was used in subsequent steps without further purification. Analytical UPLC-MS (Method 2): Retention time=1.06 min, m/z (ES+) (M+H)⁺: 330.10 (theoretical); 330.33 (observed).

Compound 27c prepared above was dissolved in anhydrous DMF (1.0 mL) and TSTU (14.05 mg, 0.066 mmol) and DIPEA (0.029 mL, 0.164 mmol) were added to it. The mixture was stirred at RT for 1 h to yield compound 27d, which was not isolated. Amino-PEG12 (9b, 36.23 mg, 0.065 mmol) and DIPEA (0.028 mL, 0.162 mmol) were added, and the reaction mixture was stirred at RT for an additional 2 h. The solvent was removed in vacuo, and the resulting residue was taken up in MeOH (2 mL). Compound 27 (34.7 mg, 73.9% yield) by isolated by flash column chromatography using a silica gel column and elution using a gradient of 0-25% MeOH in DCM. Analytical UPLC-MS (Method 2): Retention time=1.33 min, m/z (ES+) (M+H)⁺: 871.45 (theoretical); 871.58 (observed).

### Synthesis of 1-benzhydryl-1H-pyrrole-2,5-dione (28)

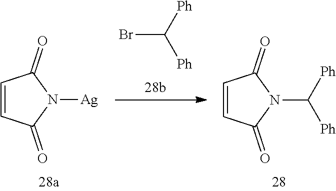

A 20-mL vial equipped with a stir bar was charged with silver maleimide (28a, 100 mg, 0.49 mmol) and anhydrous benzene (5 mL) at RT. Bromodiphenylmethane (28b, 121 mg, 0.49 mmol) was added and the reaction mixture was heated at reflux for 2 h. The reaction mixture was allowed to cool, filtered and solvent was removed in vacuo to yield crude compound 28, which was isolated by preparative HPLC (Method 4). (129 mg, 46% yield) Analytical UPLC-MS (Method 2): Retention time=2.05 min, m/z (ES+) (M+Na)⁺: 286.08 (theoretical); 286.06 (observed).

### Synthesis of 1-trityl-1H-pyrrole-2,5-dione (29)

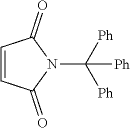 [SMILES: O=C1C=CC(=O)N1C(c1ccccc1)(c1ccccc1)c1ccccc1]

Compound 29 was prepared using similar procedures as those used to prepare compound 28. (61 mg, 73% yield) Analytical UPLC-MS (Method 2): Retention time=1.48 min, m/z (ES+) (M+Na)⁺: 362.12 (theoretical); 362.07 (observed).

### Synthesis of 3-fluoro-1-hexyl-1H-pyrrole-2,5-dione (30)

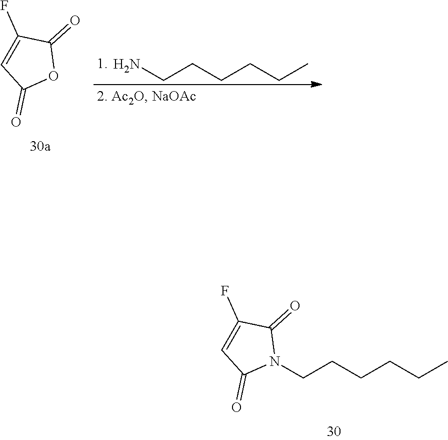

A 4-mL glass vial equipped with a stir bar was charged with 2-fluoromaleic acid (30a, 15 mg, 0.13 mmol), hexylamine (7.7 mg, 0.13 mmol), and glacial acetic acid (1 mL). The mixture was stirred at RT for 1 h. The solvent was removed in vacuo and the resulting residue was taken up in DCM (500 to which was added cold hexanes which resulted in precipitation. The precipitate was collected by filtration.

A 4-mL glass vial equipped with a stir bar was charged with the precipitate from above (6 mg, 0.028 mmol), sodium acetate (1.13 mg, 0.014 mmol), and acetic anhydride (130 uL, 1.38 mmol). The mixture was heated to 60° C. and stirred for 1 h. The solvent was removed in vacuo and compound 30 (5 mg, 82% yield) was isolated by preparative HPLC (Method 3). Analytical UPLC-MS (Method 2): Retention time=1.91 min, m/z (ES+) (M+H)⁺: 200.10 (theoretical); 200.08 (observed).

### Synthesis of 3-(2-methylene-5-oxo-2,5-dihydro-1H-pyrrol-1-yl)propanoic acid (31)

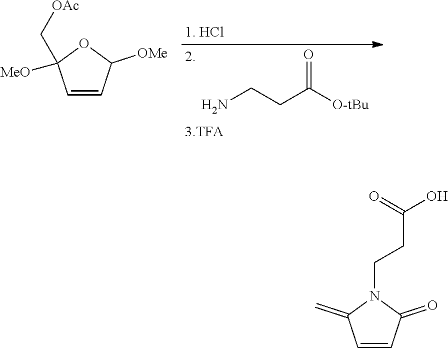

Compound 31 was synthesized using the procedures as reported in J. Am. Chem. Soc. 2017, 139, 6146-6151.

A 4-mL glass vial equipped with a stir bar was charged with (2,5-dimethoxy-2,5-dihydrofuran-2-yl)methylacetate (100 mg, 0.5 mmol) and 2 mL of 0.1 M HCl. The reaction mixture was stirred at RT for 3 h, and solid NaHCO₃(16 mg, 0.2 mmol) was added to neutralize the solution. HEPES buffer (0.5 M, pH 7.5, 0.2 mL) and tert-butyl 3-aminopropanoate (60 mg, 0.99 mmol) was added and the mixture was stirred at RT for 1 h, upon which the reaction mixture was concentrated in vacuo. The concentrated reaction mixture was diluted with DCM (10 mL), the organic layer separated, and washed with brine (2×10 mL). The organic layer was concentrated in vacuo resulting in a residue. This residue was dissolved in 30% (v/v) aqueous TFA (1 mL) and the solution was stirred at RT for 3 h. The solvent was removed in vacuo and compound 31 (15 mg, 18% yield) was isolated by preparative HPLC (Method 4). Analytical UPLC-MS (Method 2): Retention time=1.25 min, m/z (ES+) (M+H)⁺: 168.07 (theoretical); 168.07 (observed).

### Synthesis of 1-(2-hydroxyethyl)-5-methylene-1,5-dihydro-2H-pyrrol-2-one (32)

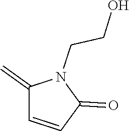

Compound 32 was synthesized using similar procedures as those used to prepare compound 31, with 2-aminoethanol in place of tert-butyl 3-aminopropanoate (12 mg, 17% yield). Analytical UPLC-MS (Method 2): Retention time=0.99 min, m/z (ES+) (M+H)⁺: 162.05 (theoretical); 162.06 (observed).

### Synthesis of (S)—N-((18S,21S,27S)-1-amino-21-isobutyl-18,29-dimethyl-4,14,17,20,23,26-hexaoxo-7,10-dioxa-3,13,16,19,22,25-hexaazatriacontan-27-yl)-1-(6-(2,5-dioxo-2,5-dihydro-1H-pyrrol-1-yl)hexanoyl)pyrrolidine-2-carboxamide (33)

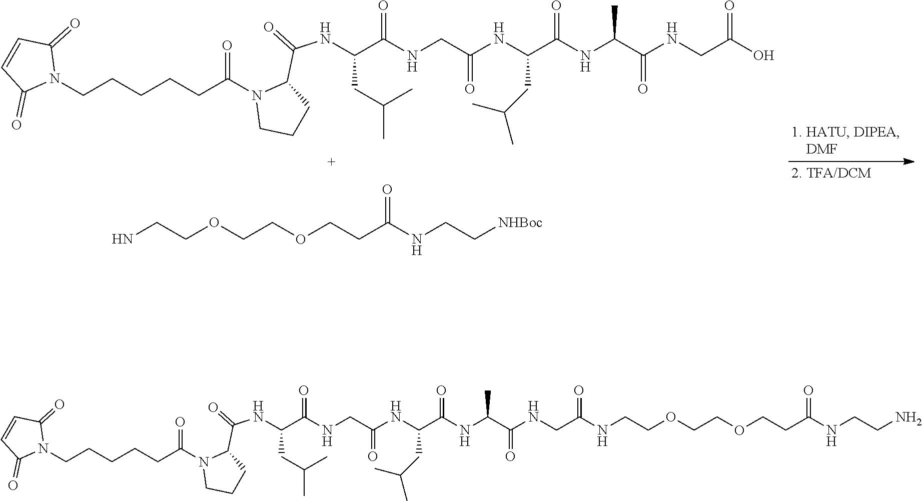 [SMILES: CC(C)C[C@H](NC(=O)CNC(=O)[C@H](CC(C)C)NC(=O)[C@@H]1CCCN1C(=O)CCCCCN1C(=O)C=CC1=O)C(=O)N[C@@H](C)C(=O)NCC(=O)O ; CC(C)(C)OC(=O)NCCNC(=O)CCOCCOCCN ; CC(C)C[C@H](NC(=O)CNC(=O)[C@H](CC(C)C)NC(=O)[C@@H]1CCCN1C(=O)CCCCCN1C(=O)C=CC1=O)C(=O)N[C@@H](C)C(=O)NCC(=O)NCCOCCOCCC(=O)NCCN]

A 4-mL glass vial equipped with a stir bar was charged with a peptide comprising a matrix metalloproteinase cleavage sequence, (6-(2,5-dioxo-2,5-dihydro-1H-pyrrol-1-yl)hexanoyl)-L-prolyl-L-leucylglycyl-L-leucyl-L-alanylglycine (20 mg, 0.027 mmol), as well as HATU (10 mg, 0.027 mmol), DIPEA (20 μL, 0.108 mmol) and DMF (300 μL). Tert-butyl (2-(3-(2-(2-(12-azaneyl)ethoxy)ethoxy)propanamido)ethyl)carbamate (9 mg, 0.0.27 mmol) was added, and the reaction mixture was stirred at RT for 4 h. The reaction mixture was then concentrated in vacuo, and then dissolved in 10% TFA in DCM (3 mL). This reaction was stirred for 1 h and concentrated in vacuo. The resulting residue re-dissolved in DMSO (2 mL) and loaded onto a preparative HPLC. Product was isolated using Method 3 (11 mg, 44% yield). Analytical UPLC-MS (Method 1): Retention time=1.35 min, m/z (ES+) (M+H)⁺: 921.54 (theoretical); 921.97 (observed).

### Example 3

### General Procedures for Attachment of Maleimide-Containing Cleavable Moieties to Antibodies

Full reduction of all interchain disulfide bonds of the antibody followed by covalent attachment of a cleavable moiety with a maleimide to the resulting reduced interchain disulfide bond thiol groups was performed as follows: 12 molar equivalents relative to antibody (or 1.5 molar equivalents to the antibody interchain disulfide bonds) of TCEP (tris 2-carboxyethyl phosphine) or DTT (dithiothreitol) were added to the antibody formulated in 1×PBS pH 7.4 with 5 mM EDTA (ethylenediaminetetraacetic acid). The solution was incubated at 37° C. for one hour. Full reduction of the interchain disulfide bonds was confirmed by PLRP-MS. Excess reductant was removed by diluting the reaction mixture into 1×PBS with 5 mM ETDA, followed by diafiltration using a low molecular weight (30 kDa) cutoff filter. Conjugation to the various maleimide intermediates was performed by adding ten molar equivalents of the maleimide intermediate relative to the antibody to the disulfide-reduced antibodies, and then incubating the reaction mixture at RT for 30 minutes. Degree of maleimide intermediate loading was determined by PLRP-MS. Excess maleimide intermediate was removed by diafiltration in 1×PBS using a low molecular weight cutoff (30 kDa) filter. The pH of the resulting modified antibody mixture was adjusted to pH 8.0 and incubated at RT overnight to force hydrolyze the succinimide groups.

### Example 4

### Succinimide Stability Assays

To assess succinimide hydrolysis and stability of the modified antibody in plasma, the MEF antibodies prepared as described above were incubated in rat plasma for between 0 and 7 days. The antibodies were purified from the rat plasma using anti-human Ab capture resin, reduced with DTT, and analyzed by PLRP-MS. An increase of 18 daltons in the m/z of the antibody light chain (LC) with BPM peak is indicative of succinimide hydrolysis. Stability of the BPMs in the modified antibodies were assessed by comparing the BPM to antibody ratio at each time point, as measured by PLRP-MS. BPM loss was assessed by the change in mass that results from deconjugation from the antibody LC or HC. BPM loss was calculated as a percentage of total BPM remaining based on total maleimide present at t=0 as described herein.

### Capillary Gel Electrophoresis Analysis of Antibody-BPM Stability

The extent of antibody disulfide re-oxidation upon release of BPMs was assessed using a Protein Simple WES® instrument. Antibodies or modified anti-CD40 antibodies that had been incubated in rat plasma in vivo or ex vivo were purified using IgSelect Protein A resin and then analyzed using a 12-230 kDa WES capillary electrophoresis separation system. Antibodies were diluted to 8 μg/mL in tris-buffered saline-Tween 20 (TBS-T) buffer, separated by capillary electrophoresis, and detected using a biotinylated F(ab′)₂ fragment goat anti-human primary antibody (10 μg/mL, Jackson ImmunoResearch) and streptavidin-poly-HRP40 secondary detection (10 μg/mL, Fitzgerald Industries).

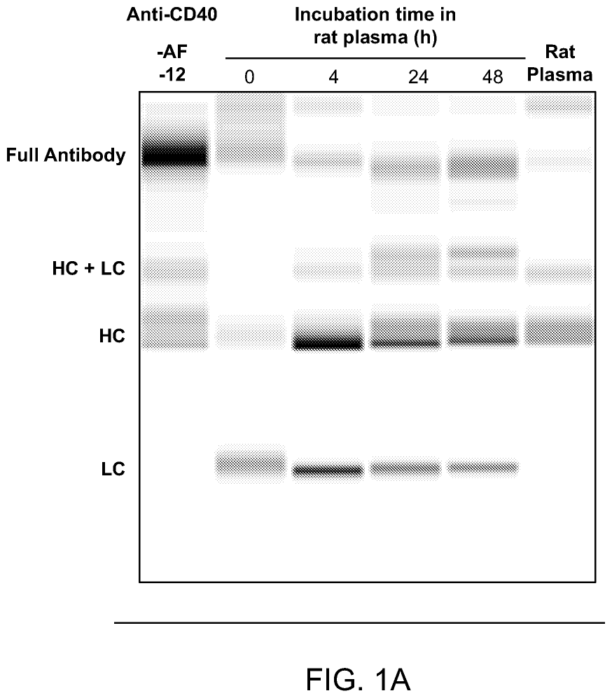

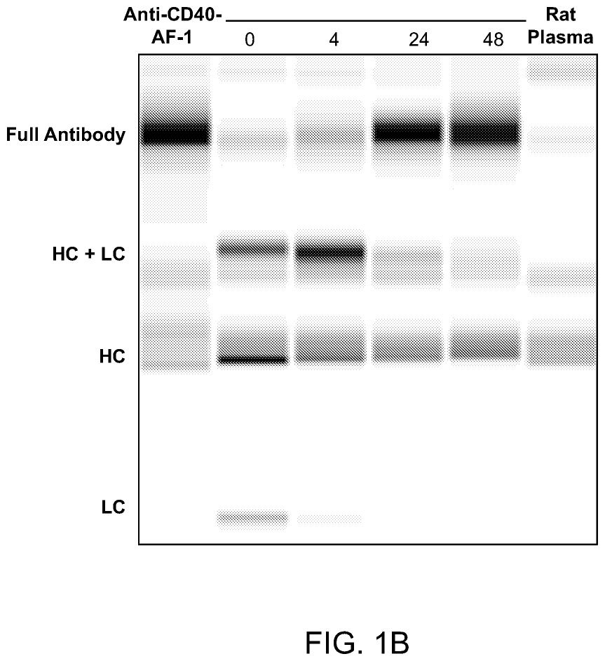

The extent of antibody re-oxidation upon release of BPMs is depicted in FIGS. 1A-B. Upon incubation of the Ab-BPM conjugates in rat plasma and de-conjugation of the BPMs, increasing band densities for antibody HC+LC and full antibody were observed compared to t=0. For Anti-CD40-AF-12 (FIG. 1A), an increase of HC+LC species were observed by 24 and 48 hr, indicating only partial BPM deconjugation and restoration of antibody interchain disulfides. For Anti-CD40-AF-1 (FIG. 1B), more complete deconjugation of the BPM led to the formation of intact antibody at the 24 and 48 hr time points.

### Example 5

### In Vitro Assays

CD16a Competitive Binding Assay Measurement of Kinetic Binding Parameters (kₒₙ, k_{off} and K_{D}) Using Biolayer Interferometry

Kinetic binding assays were performed using a ForteBio Octet RED384 instrument. Recombinant hCD16 158V monomeric Fc proteins were produced using transient expression in Chinese hamster ovary (CHO) cells. The hCD16 protein was incubated with N-hydroxysuccinimidobiotin (NHS-Biotin) at a 1:1 (mol/mol) ratio for 1 hr at RT in 1×PBS, pH 8 to introduce biotin groups. Biotinylated hCD16 protein was loaded on SAX (High sensitivity streptavidin) tips at 0.8 nM loading density. Affinity measurements were run in the kinetic buffer comprising 1×PBS, 0.1% BSA, 0.02% Tween20, pH 7.4. Association measurements were performed for 300 seconds and disassociation measurements were performed for 600 seconds. Each curve was reference subtracted and modeled using a 1:1 global fit. K_{D} results are reported as k_{off} divided by kₒₙ.

The binding kinetics antibodies modified with various BPMs (e.g. 2-8) with human Fc receptors (FcγRI, FcγRIIa H131, FcγRIIa R131, FcγRIIIa F158, and FcγRIIIa V158) were assessed by BLI (Biolayer interferometry) using ForteBio Octet RED384 and HTX instruments. Biotinylated avidin-tagged human FcγR-monomeric Fc N297A LALA-PG and FcRN monomeric Fc N297A IHH fusion proteins (designed and expressed at Seagen) were loaded onto high precision streptavidin biosensors (ForteBio) until responses between 0.3-1 nm were reached, following a 100-second sensor check in Buffer A (0.1% BSA, 0.02% Tween20, 1×PBS pH 7.4). After another baseline, titrated antibodies were associated for 600, 10, 100, 50, and 10 seconds and dissociated for 1000, 50, 100, 500, and 50 seconds in Buffer B (1% casein, 0.2% Tween20, 1×PBS pH 7.4) for FcγRI, IIa, IIIa, FcRn pH 6, and FcRn respectively. Prior to analysis, the corresponding reference curve was subtracted from each sample curve. All the sensorgrams were processed with a Y-axis alignment at the end of the second baseline and an inter-step dissociation correction. A 1:1 Langmuir isotherm global fit model was used to fit the curves.

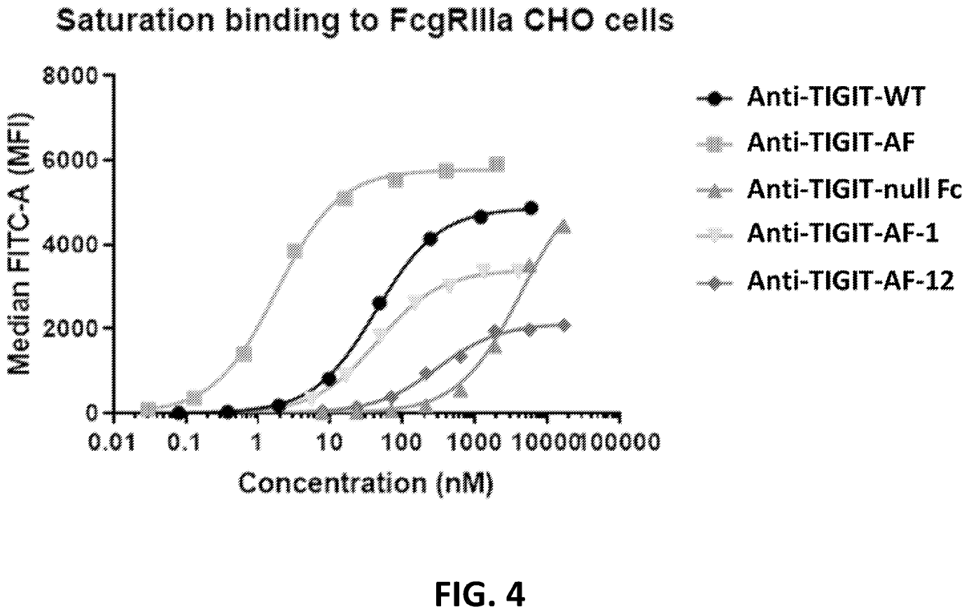

Binding data generated by BLI, presented in Tables 1 and 3, demonstrate that introduction of BPMs with increasing PEG length or bulk results in increasingly attenuated binding to all Fc gamma receptors with little impact on FcRn binding. In addition, saturation binding on CHO cells expressing human FcgRIIIa demonstrates that BPM conjugation attenuates FcgRIIIa binding. Conjugation of 1 to Anti-TIGIT-AF results in binding that is similar to Anti-TIGIT-WT, whereas conjugation of 12 to Anti-TIGIT-AF attenuates binding in a similar manner to Fc amino acid point mutations designed to minimize antibody FcgR binding (Anti-TIGIT-null Fc) (FIG. 4).

TABLE 1
Binding affinity of antibodies and 8-load PEG-BPM modified
antibodies to FcgRIIIa measured by biolayer interferometry.

| Example | PEG length | K_{D} (nM) |
|---|---|---|
| Anti-CD40-AF | — | 11 |
| (afucosylated) | | |
| Anti-CD40-AF-NEM | — | 53 |
| Anti-CD40-AF-17 | (PEG4)₂ | 75 |
| Anti-CD40-AF-14 | PEG8 | 169 |
| Anti-CD40-AF-12 | PEG12 | 197 |
| Anti-CD40-WT | — | 232 |
| Anti-CD40-AF-15 | PEG24 | 234 |
| Anti-CD40-AF-16 | PEG36 | 776 |
| Anti-CD40-AF-10 | PEG48 | 665 |
| Anti-CD40-AF-19 | PEG4-(PEG8)₃ | 913 |
| Anti-CD40-AF-20 | PEG4-(PEG24)₃ | >>1 |

TABLE 2
In vitro EC₅₀ values (μg/mL) of antibodies and 8-load modified
antibodies to FcgRIa, FcgRIIa, and FcgRIIIa by NFAT activity assay.

| Example | FcgRIa | FcgRIIa | FcgRIIIa |
|---|---|---|---|
| Anti-CD40-WT | 0.09 | 0.08 | 0.16 |
| Anti-CD40-AF | 0.07 | 0.09 | 0.05 |
| Anti-CD40-AF-9 | 0.35 | 14.31 | 0.22 |
| Anti-CD40-AF-10 | 3.73 | 227.47 | 2.09 |

TABLE 3
Binding affinity values of antibodies and 8-load modified
antibodies to FcgRIIIa measured by biolayer interferometry.

| | | FcgRIIa | FcgRIIa | | FcgRIIIa | FcgRIIIa | FcRn | FcRn |
|---|---|---|---|---|---|---|---|---|
| | FcgRIa | H131 | R131 | FcgRIIb | F158 | V158 | pH 6 | pH7.4 |
| Example | (nM) | (μM) | (μM) | (μM) | (nM) | (nM) | (nM) | (μM) |
| Anti- | 0.57 | 8.8 | 6.8 | 24 | 390 | 75 | 24 | 2 |
| CD40-AF | | | | | | | | |
| Anti-CD40- | 0.65 | 8.0 | 7.0 | 21 | 370 | 85 | 25 | 25 |
| AF-1(2) | | | | | | | | |
| Anti-CD40- | 0.49 | 9.9 | 9.8 | 19 | 640 | 140 | 27 | 9.6 |
| AF-9(2) | | | | | | | | |
| Anti-CD40- | 0.62 | 8.7 | 7.7 | 22 | 540 | 130 | 26 | 10 |
| AF-1(4) | | | | | | | | |
| Anti-CD40- | 0.9 | 19 | 13 | 64 | 1400 | 360 | 26 | 13 |
| AF-9(4) | | | | | | | | |
| Anti-CD40- | 1.4 | 15 | 15 | 25 | 970 | 220 | 29 | 10 |
| AF-1(6) | | | | | | | | |
| Anti-CD40- | 1.7 | 27 | 21 | 97 | 2600 | 710 | 28 | 11 |
| AF-9(6) | | | | | | | | |
| Anti-CD40- | 3.8 | 25 | 27 | >100 | 6500 | 2500 | 31 | 9 |
| AF-1(8) | | | | | | | | |
| Anti-CD40- | 1.5 | 21 | 18 | >100 | 5800 | 2800 | 32 | 11 |
| AF-9(8) | | | | | | | | |
| Anti-CD40- | 1.2 | 24 | 22 | 56 | 3900 | 1300 | 28 | 15 |
| AF NEM | | | | | | | | |

Anti-CD40-AF-1 = Anti-CD40-AF conjugated to Compound 1, disulfide-PEG12
Anti-CD40-AF-9 = Anti-CD40-AF conjugated to Compound 9, maleimidopropionyl-PEG12

### PBMC Cytokine Stimulation

Frozen human PBMCs obtained from Astarte Biologics were incubated with increasing concentrations of Anti-CD40-WT, Anti-CD40-AF, or modified Anti-CD40-AF antibodies in a 96-well tissue culture plate for 6-24 hours at 37° C. in 8% CO₂. PBMCs were then spun with a plate adapter at 800 rpm for 5 min. Tissue culture supernatant was removed and transferred to a 96-strip tube rack and samples were frozen at −80° C. until further processing.

Cytokine production was monitored using a Luminex Multiplex Kit from Millipore (HCYTOMAG-60K). Tissue culture supernatants and serum samples were processed as per the manufacturer's instructions. Briefly, assay plates were washed with 200 μL of wash buffer per well, followed by addition of 25 μL standard or buffer, 25 μL matrix or sample, and 25 μL of multiplexed analyte beads to each well. Samples were incubated overnight with vigorous shaking using an orbital shaker at 4° C. The assay plates were washed twice with wash buffer. To each well was added 25 μL of a solution containing the detection antibodies, and the assay plates were incubated at RT for 1 hour. Thereafter, 25 μL of a solution containing streptavidin-phycoerythrin (SA-PE) were added and the assay plates were incubated at RT for 30 minutes. The plates were washed twice with wash buffer and beads were resuspended with 150 μL of sheath fluid. The samples were analyzed using Luminex MagPix systems using the Xponent software. Cytokine levels were measured against the standard curve generated within each experiment.

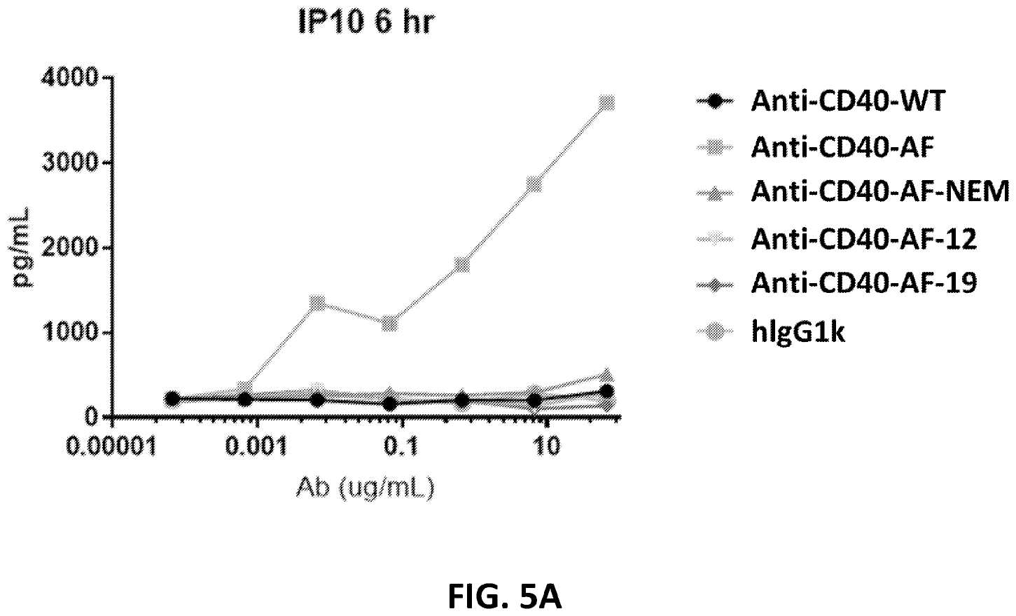

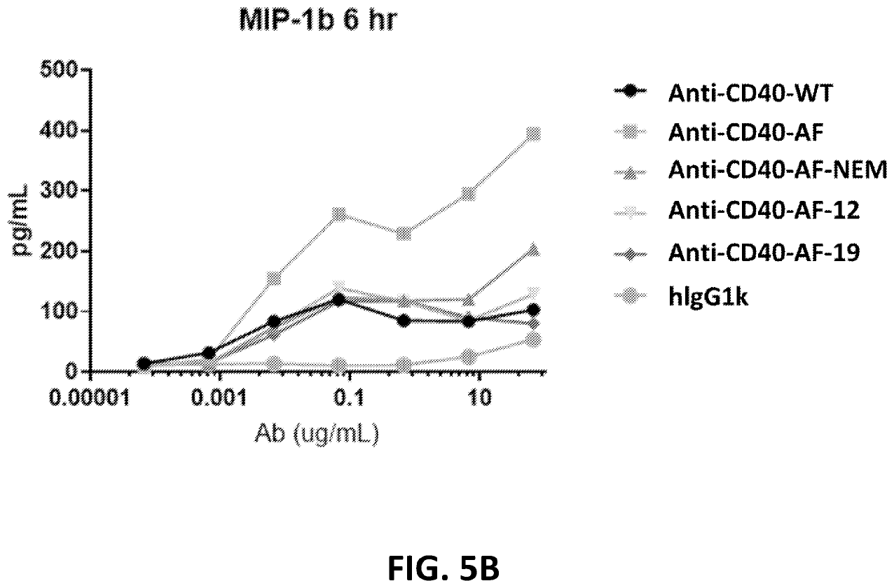

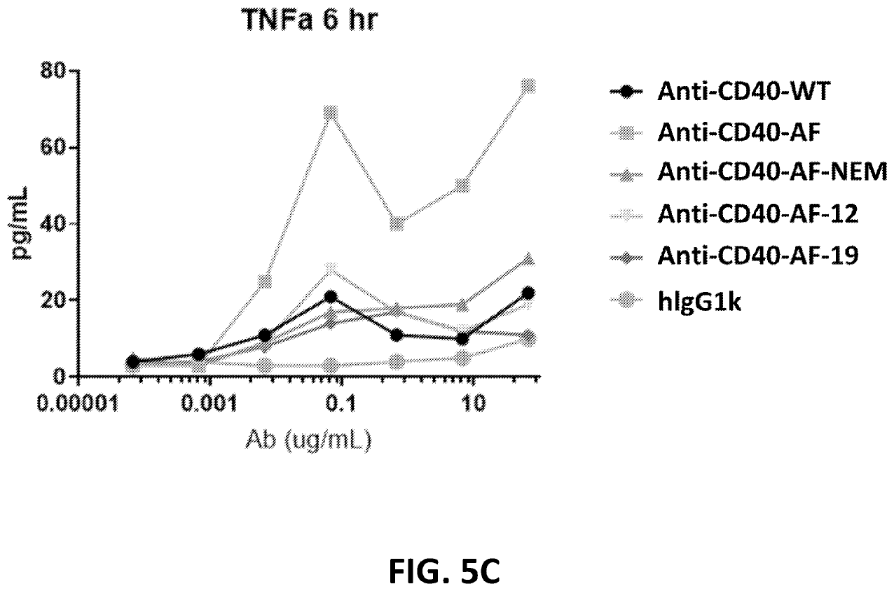

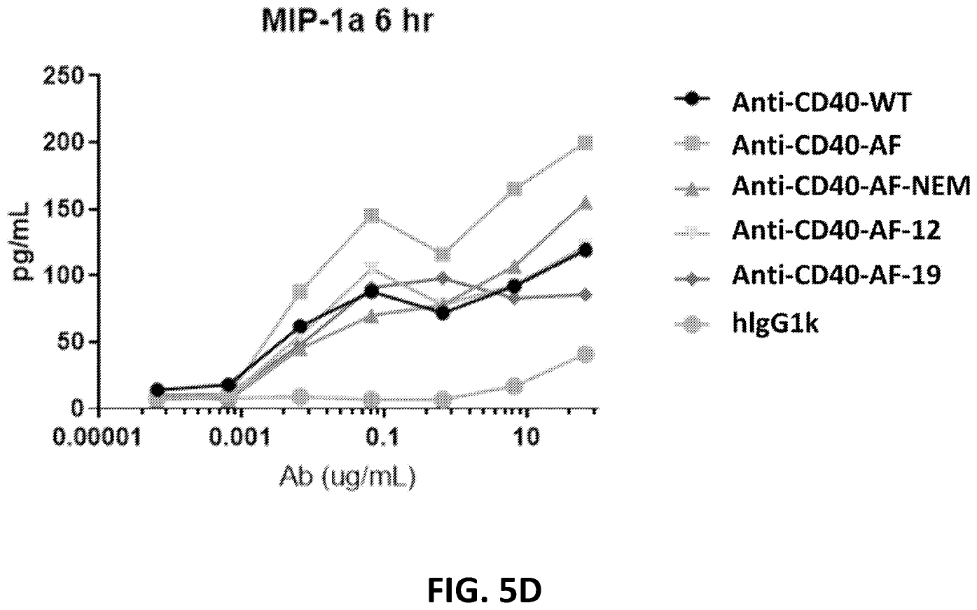

FIGS. 5A-D summarize cytokine responses resulting from incubation of Anti-CD40-WT, Ab-AF, Ab-AF-NEM, Anti-CD40-AF-12, Anti-CD40-AF-19, and human IgG1k isotype control with human PBMCs for 6 hours. Anti-CD40-AF results in increased production of IP10 (FIG. 5A), MIP-1β (FIG. 5B), TNFα (FIG. 5C), and MIP-1α (FIG. 5D) production in comparison to Anti-CD40-WT. Conjugation of BPMs 12 or 19 to Anti-CD40-AF attenuates production of IP10, TNFα, and MIP-1α to levels that are similar to Anti-CD40-WT.

### CD16a NFAT Signaling Assay

WIL2-S target cells were diluted to a density of 1.5×10⁶ cells/mL in pre-warmed RPMI 1640 cell culture media containing 4% Super Low IgG Defined FBS and 25 μL of cells were plated in each well of a conical bottom 96-well plate. To the WIL2-S cells were added serial dilutions of antibody or modified antibody (25 μL per well) and the plates were shaken at RT at 300 rpm for approximately 5 min on an orbital shaker. During this time, Jurkat NFAT CD16a (FcγRIIIa) cells were suspended in low IgG media to a density of 3.0×10⁶ cells/mL. 25 μL of a suspension containing Jurkat NFAT effector cells were then transferred to each well of the plate and the samples were incubated at 37° C., 5% CO₂ for 4-24 hours. Thereafter, 75 μL of Bio-Glo luciferase assay reagent was added to each well and the luminescence was measured using an Envision multilabel plate reader.

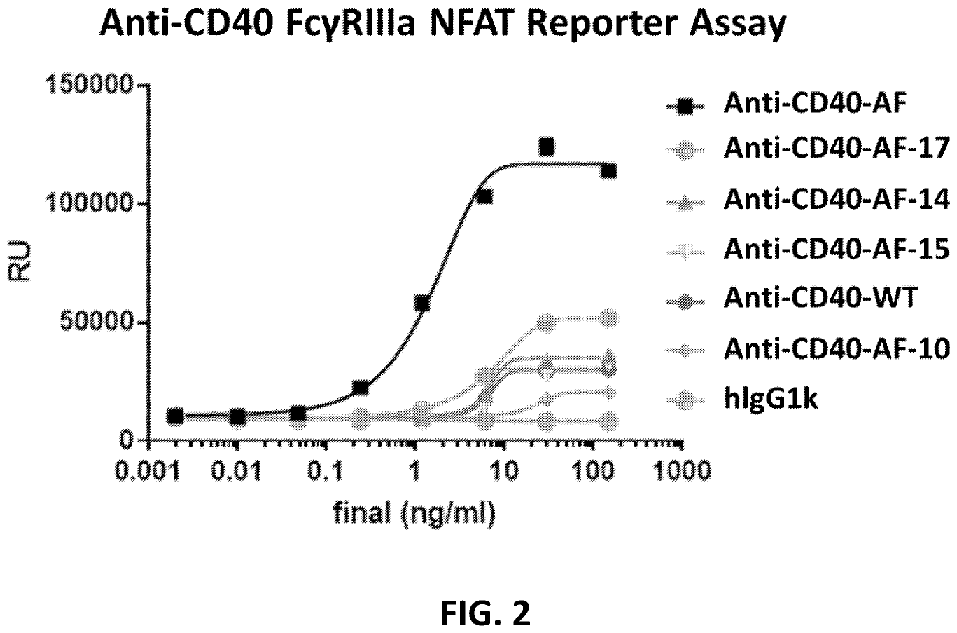

FIG. 2 depicts the impact of BPM moieties with different PEG lengths or bulk on the signaling of Anti-CD40-AF antibodies in an FcγRIIIa NFAT signaling assay. The conjugates were compared to Anti-CD40-WT, Anti-CD40-AF, and hIgG1k isotype control antibodies. Conjugates with impaired FcγRIIIa binding displayed minimal signaling, similar to Anti-CD40-WT, and signaling was further impaired in a manner consistent with the measured FcγRIIIa affinity.

FIGS. 3A-B depict the impact of BPM 1 or 12 conjugation to Anti-CD40-AF in a FcgRIIIa NFAT signaling assay, before and after incubation in rat plasma for up to 48 hours. The BPM conjugates were compared to Anti-CD40-WT, Anti-CD40-AF, and hIgG1k isotype control antibodies. At time 0, both Anti-CD40-AF-1 and Anti-CD40-AF-12 display limited signaling through FcgRIIIa, similar to Anti-CD40-WT. Over the course of 48 hours, as BPM deconjugation occurs, signaling is increased. For Anti-CD40-AF-1 signaling at 24-48 hours is comparable to Anti-CD40-AF. Meanwhile, signaling of Anti-CD40-AF-12 at 48 hours is still diminished compared to Anti-CD40-AF due to incomplete deconjugation and restoration of antibody interchain disulfide bonds. FIG. 3C depicts the impact of BPM 12 conjugation to Anti-BCMA-AF in a FcgRIIIa NFAT signaling assay, before and after incubation in rat plasma for up to 5 days. Anti-BCMA-AF-12 was compared to Anti-BCMA-WT, Anti-BCMA-AF, and hIgG1k isotype control antibodies. At time 0, Anti-BCMA-AF-12 display limited signaling through FcgRIIIa, similar to Anti-BCMA-WT. Over the course of the incubation, as BPM deconjugation occurs, signaling is increased. At later timepoints (2-5 days), the extent of signaling for Anti-BCMA-AF-12 is similar in magnitude to Anti-BCMA-AF, though with a lower EC₅₀, likely indicating that some of the BPM groups are retained on the antibody interchain disulfide cysteine residues.

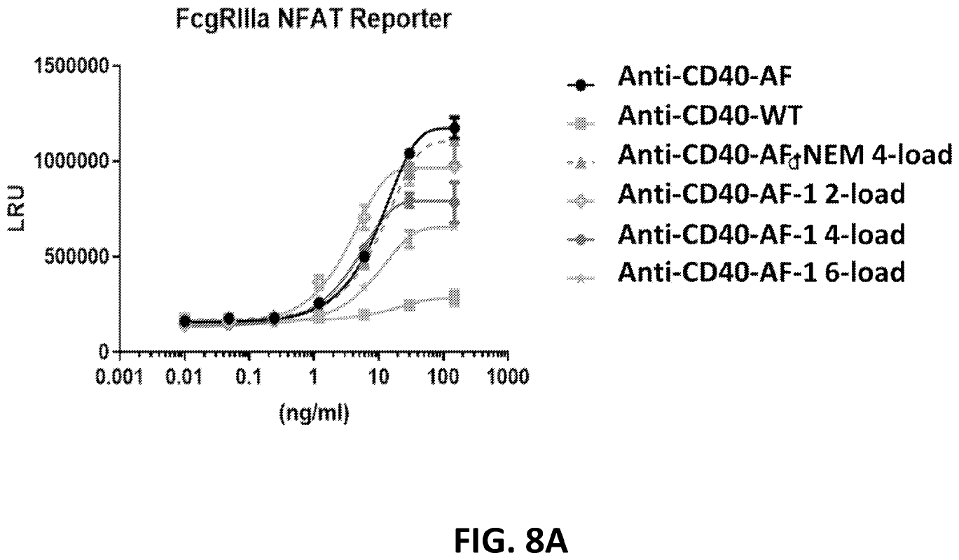

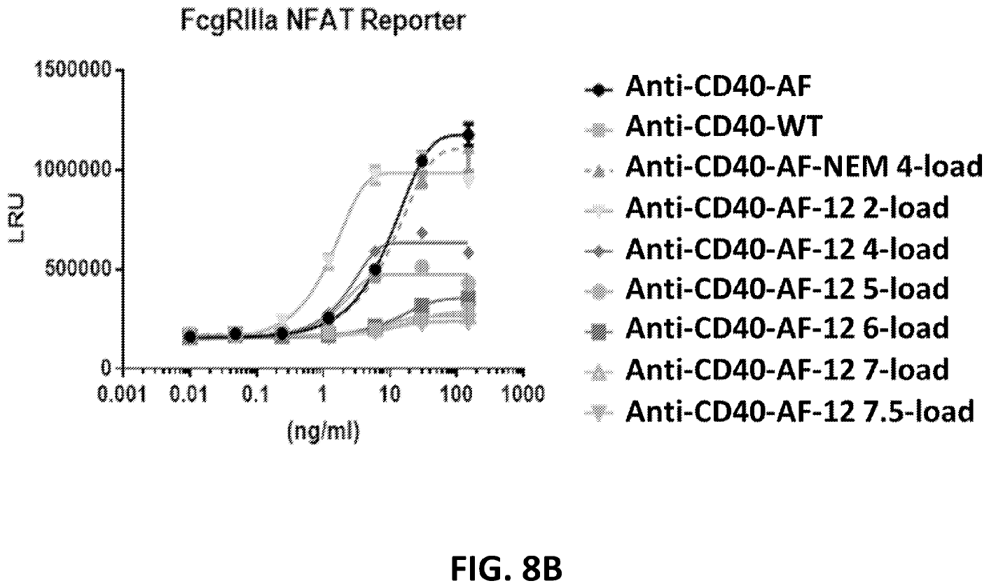

FIG. 8A depicts the impact of partial BPM conjugation to Anti-CD40-AF and its impacts on signaling in an FcγRIIIa NFAT assay. Anti-CD40-AF was conjugated with BPM 1 to achieve partial-loaded conjugates bearing 2, 4, or 6 BPM per antibody. These conjugates were compared to Anti-CD40-AF and Anti-CD40-WT antibody controls in an FcγRIIIa NFAT signaling assay. This experiment demonstrated that complete conjugation of BPM 1 is required to ablate FcγRIIIa binding and signaling, and also indicates that only minimal deconjugation of BPM 1 would be required in vivo to restore antibody binding to FcγRIIIa. In FIG. 8B, Anti-CD40-AF was conjugated with BPM 12 to achieve partial-loaded conjugates bearing 2, 4, 5, 6, 7, and 7.5 BPM per antibody. These conjugates were compared to Anti-CD40-AF and Anti-CD40-WT antibody controls in an FcγRIIIa NFAT signaling assay. This experiment demonstrated that conjugates with at least 6 BPM per antibody had minimal binding and signaling, whereas conjugates bearing few than 5 BPM per antibody begin to elicit signaling.

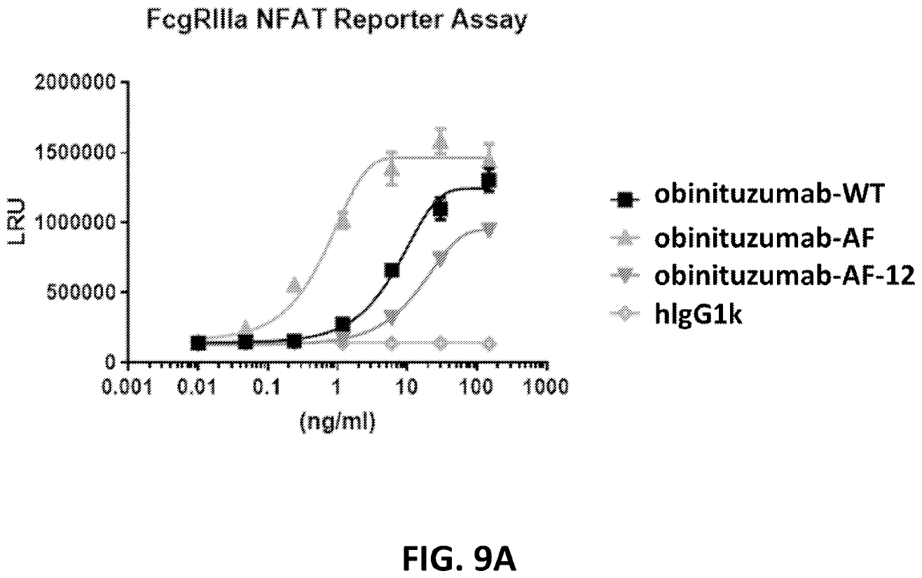

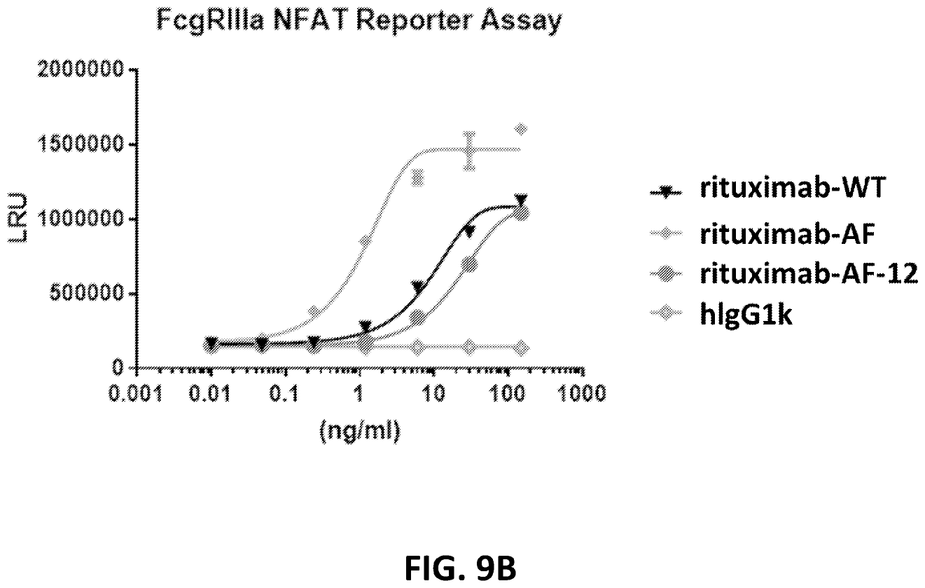

FIG. 9A depicts the impact of BPM 12 on the signaling of obinituzumab-AF antibody in an FcγRIIIa NFAT signaling assay. The conjugates were compared to obinituzumab-WT, obinituzumab-AF, and hIgG1k isotype control antibodies. obinituzumab-AF-12 displayed FcγRIIIa engagement and signaling that was diminished compared to obinituzumab-WT. FIG. 9B depicts the impact of BPM 12 on the signaling of rituximab-AF antibody in an FcγRIIIa NFAT signaling assay. The conjugates were compared to rituximab-WT, rituximab-AF, and hIgG1k isotype control antibodies. rituximab-AF-12 displayed FcgRIIIa engagement and signaling that was diminished compared to rituximab-WT.

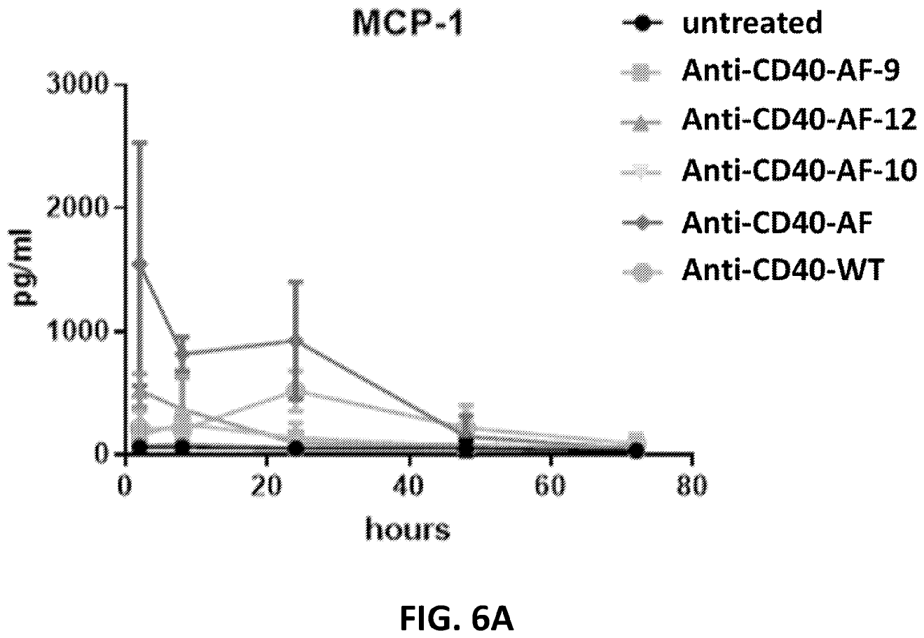

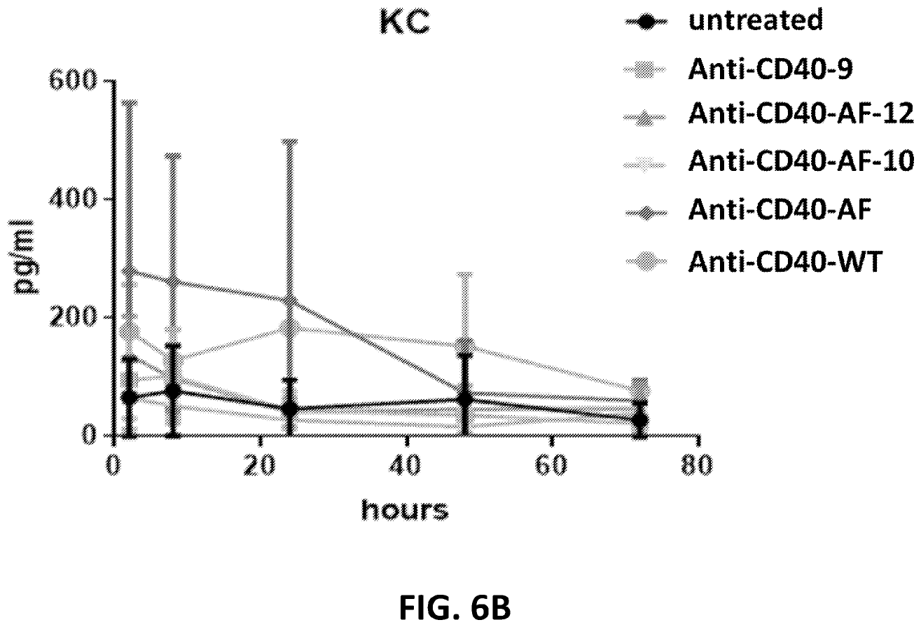

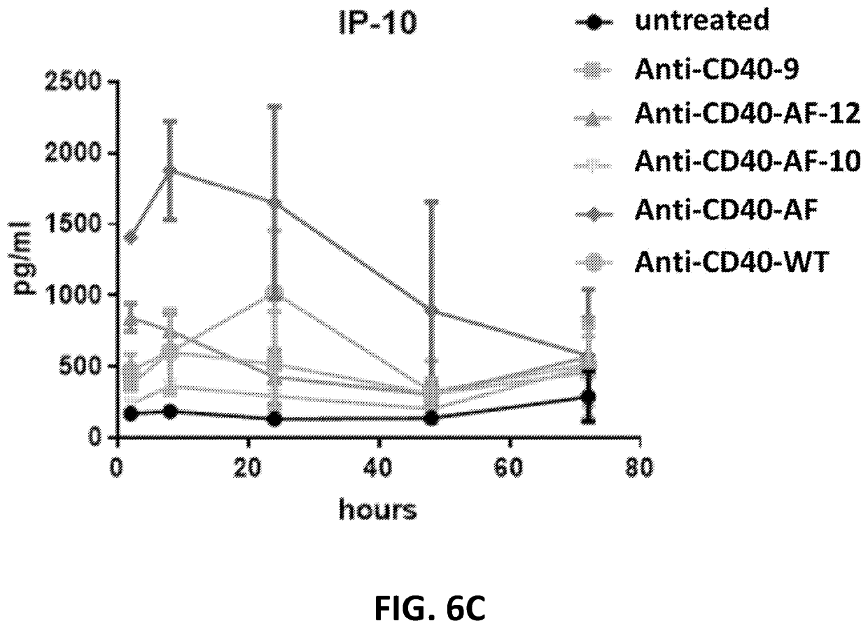

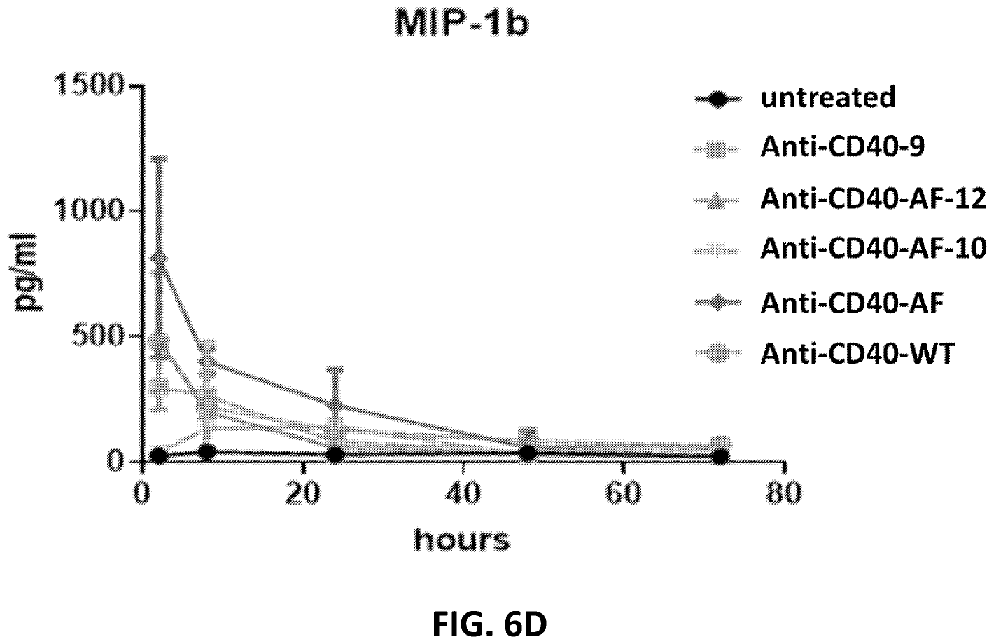

FIG. 6 depicts the cytokine response that results from in vivo dosing of Anti-CD40-AF-9, Anti-CD40-AF-10, and Anti-CD40-AF-12 into mice with the human transgenic receptor for Anti-CD40. These conjugates were compared to Anti-CD40-WT, Anti-CD40-AF, and an untreated control, and plasma samples were taken at timepoints of 2, 6, 24, 48 and 72 hours. Conjugates displayed similar cytokine release profiles as Anti-CD40-WT and the levels of cytokines were diminished in a manner consistent with the measured FcγRIIIa activity. There was no delayed response despite restoration of FcγR binding over time.

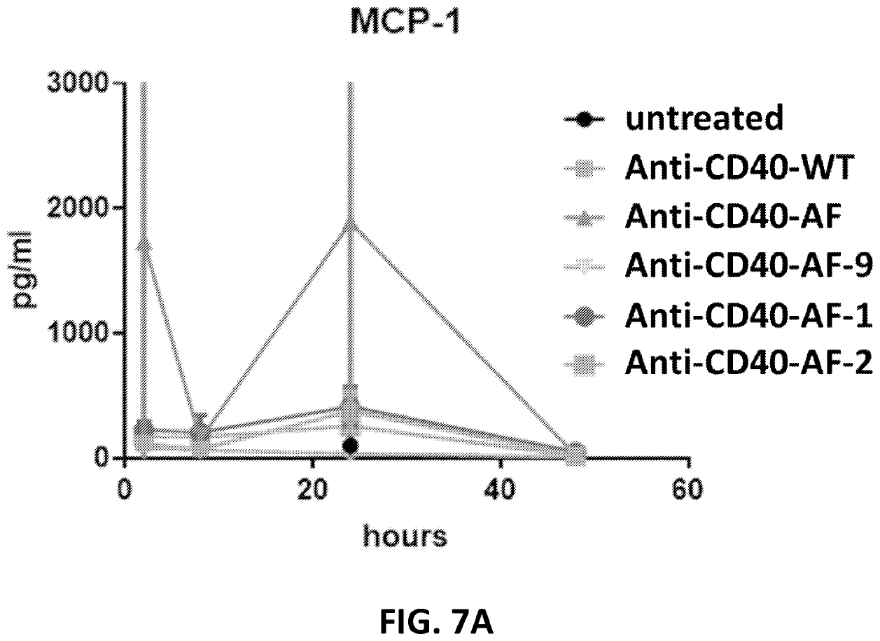

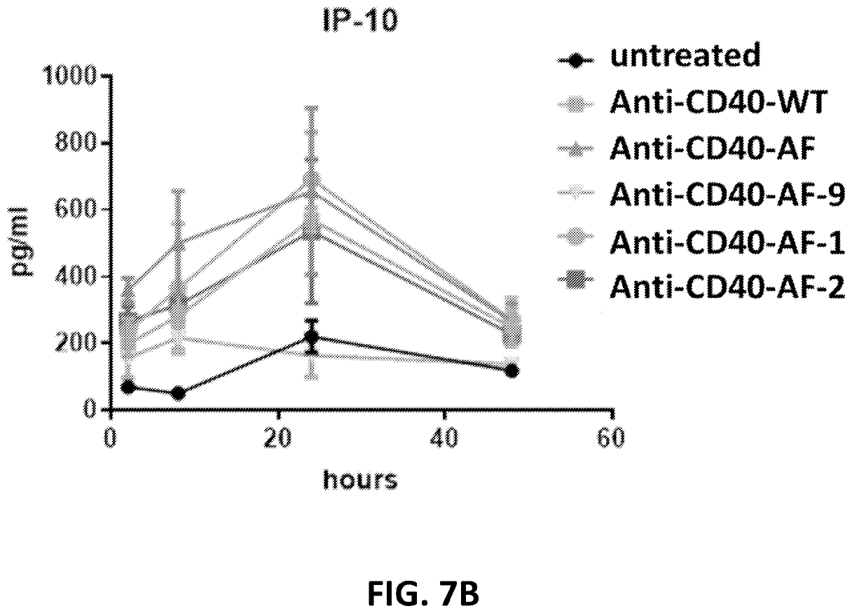

FIG. 7 depicts the cytokine response that results from in vivo dosing of Anti-CD40-AF-1, Anti-CD40-AF-2, and Anti-CD40-AF-9 into mice with the human transgenic receptor for Anti-CD40. These conjugates were compared to Anti-CD40-WT, Anti-CD40-AF, and an untreated control, and plasma samples were taken at timepoints of 2, 6, 24, and 48 hours. Conjugates displayed similar cytokine release profiles as Anti-CD40-WT and decreased cytokine levels as Anti-CD40-AF. There was no delayed response despite restoration of FcgR binding over time.

### Assessment of BPM-Conjugated Antibodies in a Murine Tumor Models:

Mouse colon cancer cells (100,000 CT26WT cells) were implanted subcutaneously to Balb/c mice. Mice were randomized into cohorts each with tumor size of approximately 50 mm³ on average. Mice were then given intraperitoneal injections of either Anti-TIGIT-WT, Anti-TIGIT-AF, Anti-TIGIT-null Fc, Anti-TIGIT-AF-1, or Anti-TIGIT-AF-12, every three days for a total of three doses. Mice were monitored until the implanted tumors reach 500 mm³, at which point they were sacrificed.

Mouse B-cell lymphoma cells (5,000,000 A20 cells) were implanted subcutaneously to Balb/c mice with human transgenic receptor binding to Anti-CD40. Mice were randomized into cohorts each with tumor size of approximately 50 mm³ on average. Mice were then given intraperitoneal injections of either Anti-CD40-WT, Anti-CD40-AF, Anti-CD40-AF-1, Anti-CD40-AF-9, or Anti-CD40-AF-12, every three days for a total of three doses. Mice were monitored until the implanted tumors reach 1000 mm³, at which point they were sacrificed.

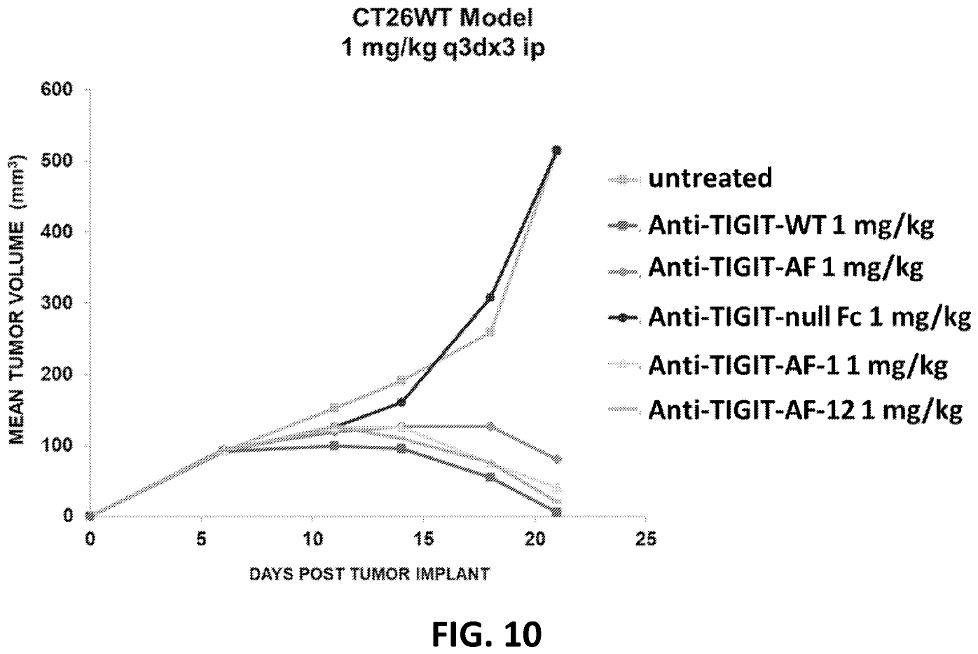

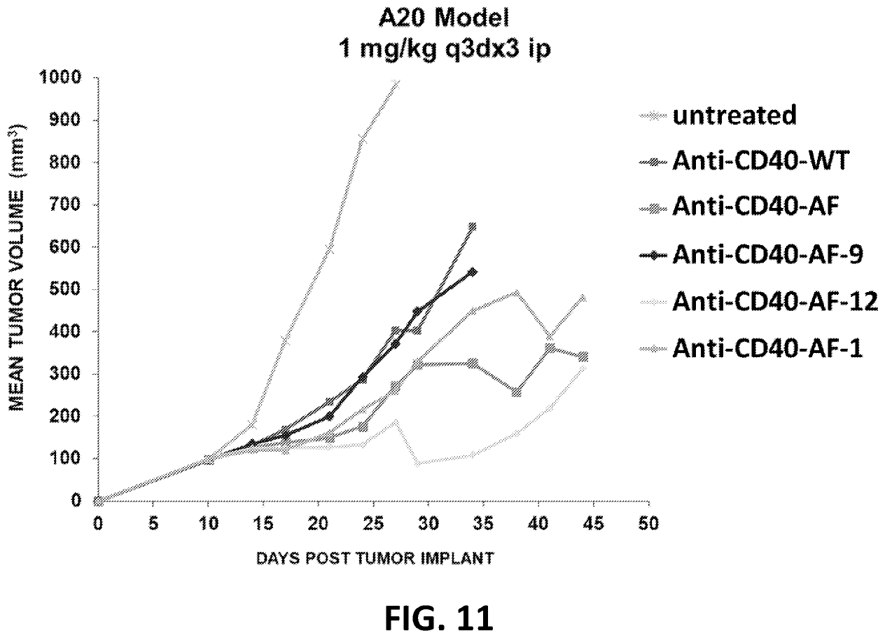

FIG. 10 depicts the tumor growth or delayed growth from in vivo dosing of Anti-TIGIT-WT, Anti-TIGIT-AF, Anti-TIGIT-null Fc, Anti-TIGIT-AF-1, or Anti-TIGIT-AF-12. Mice treated with Anti-TIGIT-AF-1 or Anti-TIGIT-AF-12 showed significant survival benefit over the Anti-TIGIT-null Fc and similar tumor delay as the parental Anti-TIGIT-AF. FIG. 11S depicts the tumor growth or delayed growth from in vivo dosing of Anti-CD40-WT, Anti-CD40-AF, Anti-CD40-AF-1, Anti-CD40-AF-9, or Anti-CD40-AF-12. Mice treated with Anti-CD40-AF-12 showed significant survival benefit over the Anti-CD40-AF, and Anti-CD40-AF-1 showed a similar tumor growth delay as Anti-CD40-AF. Anti-CD40-AF-9, a stable, force-hydrolyzed conjugate, showed similar efficacy as Anti-CD40-WT.

### Example 6

### MEF Antibody BPM Cleavage Rates and CD16a Activity

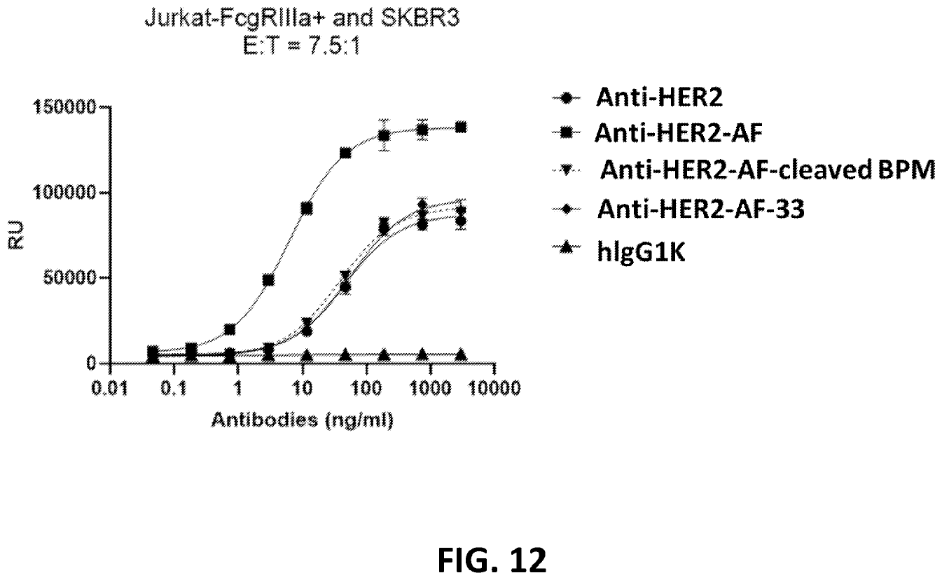

This example covers an FcγIIIa binding assay with an antibody containing PEGylated-oligopeptide functionalizations. Afucosylated Anti-HER2 antibodies were prepared with an oligopeptide-PEG-containing BPM (compound 33), the cleaved analogue of compound 33, or no functionalizations, and then utilized for FcγRIIIa activity assays as outlined in EXAMPLE 5. Results from these assays are summarized in FIG. 12. Non-BPM functionalized fucosylated and afucosylated antibodies were also interrogated. While the afucosylated, non-BPM-functionalized antibody exhibited the highest activity at all doses, the activities of the BPM-functionalized and cleaved-BPM-functionalized antibodies exhibited similar dose-response relationships.

### Example 7

### Pharmacokinetic Profiles of a PEGylated Antibody

In this example, pharmacokinetic profiles were analyzed following administration of a single intravenous dose of PEGYLATED antibody conjugates to Sprague Dawley rats. Plasma was collected and analyzed for generic total antibody (gTAb) by immunoassay and by LCMS/MS, as well as BPM-antibody ratios.

### Generic Total Antibody Measurements

Total human IgG was detected in plasma using the Gyrolab platform (Gyros AB, Sweden). Assay standards and quality control samples (QCs) were prepared using the dosed test article diluted in pooled female Sprague Dawley rat plasma. Standards, QCs, and study samples were diluted into Rexxip buffer (Gyros AB, Sweden). Briefly, a biotinylated murine anti-human IgG was captured onto streptavidin coated beads within the Gyrolab Bioaffy CD. After being captured, human IgG was detected with an Alexa Fluor 647 (Thermo Scientific) labeled goat anti-human IgG. The fluorescence signal (in Response Units) was read at the 1% photomultiplier tube (PMT) setting. Unknown sample concentrations were determined by interpolating against a standard curve fit with a 5-parameter logistic function weighted by 1/y² using the Gyrolab Evaluator Software (Version 3.6.2.30). The dynamic range of the assay is 22.9 ng/mL-10,000 ng/mL in neat plasma.

### Drug-Antibody Ratio Measurements

To directly measure the drug-to-antibody ratio (DAR) of Anti-CD40-SEA-MCPEG12 dosed in rats, in vivo plasma samples were subjected to immunoaffinity enrichment using biotinylated anti-idiotypic antibodies conjugated to paramagnetic beads. In these assays, the DAR was defined as the ratio of cleavable PEG moieties to antibodies. The assay was optimized to capture 70 μg of Anti-CD40-SEA-MCPEG12 from Sprague Dawley rat plasma diluted 1:5 in PBS-T. Plasma dilutions were determined based upon gTAb analysis. Immunocaptured ADC was eluted from the conjugated beads using a glycine-based buffer followed by alkalization to pH 8.0 using Tris (tris hydroxymethyl aminomethane) prior to deglycosylation with PNGase F. The ADC was then buffer exchanged into 50 mM ammonium acetate for subsequent intact protein analysis by nSEC-MS (native Size Exclusion Chromatography with Mass Spectrometry detection). Raw mass spectrometry files were deconvoluted using a custom protocol in automated software to provide the PEG load profile at each study timepoint that was used to calculate the drug-to-antibody ratio.

FIG. 13 summarizes results of the DAR analysis, with the inset table providing calculated DARs for plasma collected 0.042, 0.25, 1, 3, 5, and 8 days following injection. As can be seen from the plot, DAR followed an exponential decay-like profile with respect to time. At 0.042 days (approximately 1 hour) following injection, the DAR is close to 8, indicating that the majority of the 8 available thiols were coupled to PEG linkers. At 1 day, the DAR measured to 6.05, indicating that approximately 3-in-4 thiols remained coupled to PEG units. The DARs of 3.88 and 3.17 at days 5 and 8 indicate that the antibodies retained partial PEGylation, and therefore likely also exhibited diminished effector functions. These results demonstrate that MEF antibodies can be tuned for delayed and time-dependent effector functions.

Next, the effect of antibody incubation time was interrogated in a CD16a NFAT signaling assay with the PEG12 functionalized antibody. The assays were performed according to Example 5. Antibodies were collected 1 to 192 hours after administration to rats, and dosed to a co-culture of An1-positive WIL2-S target cells and Jurkat FcgRIIIa NFAT reporter cells to test the impacts on PEG deconjugation and effector function.

Results of the analyses are provided in FIG. 14, with data representing an average of 3 animals per timepoint, and each concentration and control (Anti-CD40-IgG, Anti-CD40-SEA, Isotype IgG1) run in duplicate. As can be seen in FIG. 14, the activity of the PEG12 antibody increased with incubation time, exhibiting about ⅙^{th} of the maximum activity following 1 hour of incubation than at 192 hours. Activity increased between each collection time (1 hour, 6 hours, 24 hours, 72 hours, 120 hours, and 192 hours). The lower maximum activity of the PEG12 functionalized antibody as compared to the Anti-CD40-SEA control antibody suggests that the PEG12 functionalized antibody may have regained only partial activity following 192 hours of incubation, and that the antibody may continue increasing in activity after 192 hours. The half maximal effective concentration remained fairly constant over the tested incubation time range.

### Example 8

### Effects of Antibody Mutation and PEGylation on FcγR Binding

This example covers the effects of antibody mutations and PEGylation on effector function activity generated with in CD16a NFAT assays utilizing wild-type and FcγRIIIa V158. For these analyses, Fc receptor proteins were bound tightly to the SAX sensors used for the interferometry measurements. Biotinylated recombinant human Fc receptor proteins containing either C-terminal Avi- or monomeric Fc-tags (produced at Seagen) were diluted in immobilization buffer (0.1% BSA+0.02% Tween20, 1×PBS pH 7.4) and loaded onto SAX (streptavidin) biosensors (ForteBio) with optimized conditions (TABLES 4 and 5). After a quick baseline in immobilization buffer to ensure recombinant Fc receptor proteins were bound tightly to the SAX sensors, a second baseline in kinetic buffer (1% casein+0.2% Tween20, 1×PBS pH 7.4 for all huFcγR interactions and 1% BSA+0.2% Tween20, Phosphate Citrate pH 6.0 for huFcRN interactions) was performed.

Serial dilutions of multiple MEF and variant antibodies with combinations of S239D, A330L, I332E, and PEG BPM modifications were allowed to associate with recombinant protein immobilized on biosensors until the top concentration of test articles reached equilibrium with recombinant protein. CD16a NFAT EC₅₀ values for the various antibodies are summarized in Table 4, while CD16a V158 K_{D} and k_{d} values are listed in Table 5. Lastly, biosensors were incubated in kinetic buffer to allow for antibody dissociation to occur. Sensorgrams capturing the association and dissociation of antibody from recombinant protein were generated at 30° C. on an Octet HTX system (ForteBio). Reference biosensors with immobilized recombinant protein were measured in the absence of test article. Negative control biosensors without immobilized recombinant protein were assessed with test articles present at 20 μM to verify the absence of nonspecific binding of the test articles to the SAX biosensors themselves.

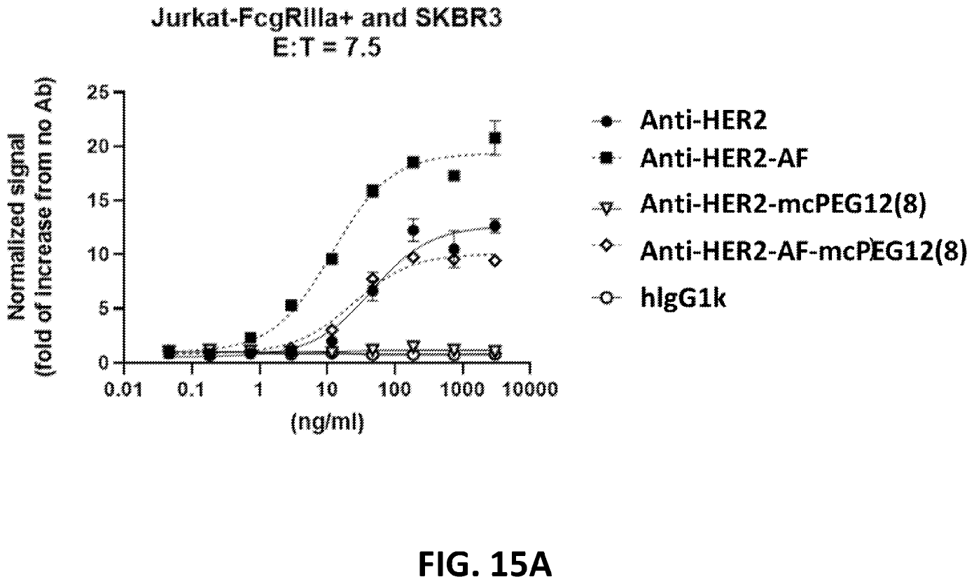

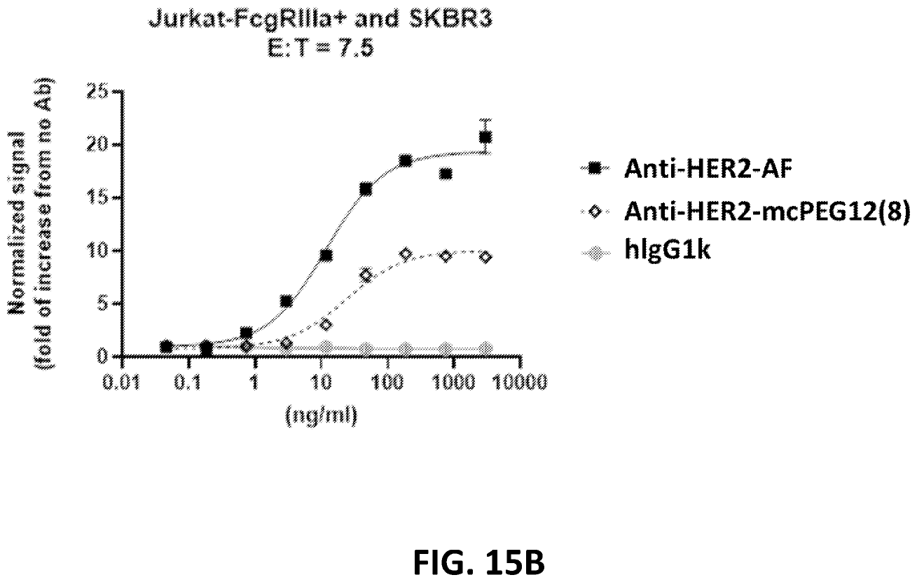

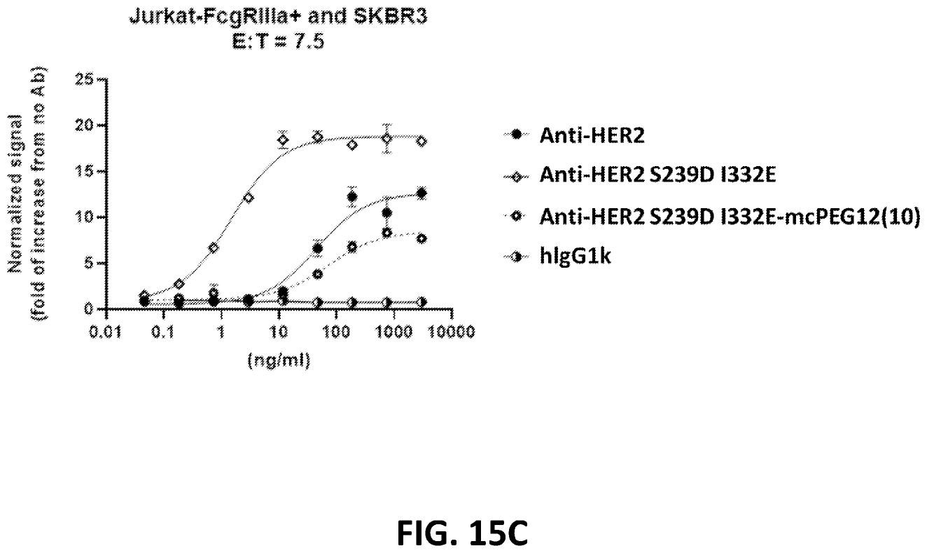

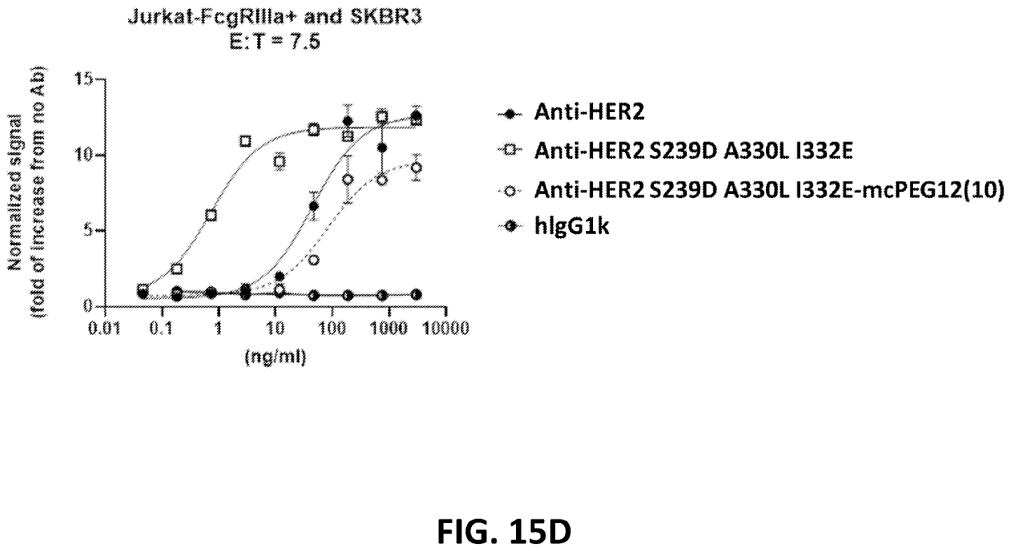

Binding results are summarized in FIGS. 15A-D and TABLES 4-8. In these tables and figures, ‘SEA’ indicates afucosylation, and ‘mcPEG12(8) and mcPEG12(10) indicate BPMs functionalizations. As shown in TABLE 4, while BPM functionalized antibodies lacking effector function enhancing modifications (e.g., afucosylation, S239D mutations, etc.) exhibited FcγRIIIa μg/mL-range EC₅₀ values, antibodies with BPMs and effector function enhancing modifications maintained FcγRIIIa activities similar to that of an unfunctionalized antibody (Anti-HER2). For V158 and F158 FcγRIIIa, FcγRIIIa variants with diminished Fc binding affinity, multiple effector function enhancing modifications (e.g., Anti-HER2 S239D I332E SEA-mcPEG12(10)) were required for BPM-functionalized antibodies to exhibit nM binding affinities. While antibodies with and without effector function enhancing modifications exhibited similar-fold decreases in FcγRI, FcγRIIIa, and variant FcγRIIIa binding affinity upon BPM functionalization, only some antibodies retained H131 FcγRIIa binding upon BPM functionalization.

TABLE 4
Wild-Type FcγRIIIa Antibody Binding

| | CD16a NFAT |
|---|---|
| Analyte | EC₅₀ (ng/mL) |
| Anti-HER2 | 44.5 |
| Anti-HER2-mcPEG12(8) | >2000 |
| Anti-HER2 SEA | 11.8 |
| Anti-HER2 SEA- mcPEG12(8) | 23.6 |
| Anti-HER2 S239D 1332E | 1.5 |
| Anti-HER2 S239D 1332E- mcPEG12(10) | 71.2 |
| Anti-HER2 S239D 1332E SEA | 2.3 |
| Anti-HER2 S239D 1332E SEA-mcPEG12(10) | 28.1 |
| Anti-HER2 S239D A330L 1332E | 0.7 |
| Anti-HER2 S239D A330L 1332E -mcPEG12(10) | 83.5 |
| Anti-HER2 S239D A330L 1332E SEA | 2.0 |
| Anti-HER2 S239D A330L 1332E SEA- mcPEG12(10) | 33.0 |

TABLE 5
Human V158 FcγRIIIa Biolayer Interferometry

| | K_{D} | K_{D} | K_{D} Fold | kₐ | k_{d} |
|---|---|---|---|---|---|
| Analyte | (M) | (nM) | change | (1/(Ms)) | (1/s) |
| Anti-HER2 | 5.7E−07 | 570 | | 8.0E+04 | 4.5E−02 |
| Anti-HER2-mcPEG12(8) | 1.8E−05 | 18000 | 31.6 | 1.3E+04 | 2.3E−01 |
| Anti-HER2SEA | 3.7E−08 | 37 | | 5.0E+05 | 1.9E−02 |
| Anti-HER2SEA- mcPEG12(8) | 1.2E−06 | 1200 | 32.4 | 9.0E+04 | 1.1E−01 |
| Anti-HER2S239D I332E | 4.3E−08 | 43 | | 7.4E+05 | 3.2E−02 |
| Anti-HER2 S239D I332E- | 2.8E−06 | 2800 | 65.1 | 4.2E+04 | 1.2E−01 |
| mcPEG12(8) | | | | | |
| Anti-HER2 S239D I332E SEA | 2.9E−09 | 2.9 | | 8.0E+05 | 2.3E−03 |
| Anti-HER2 S239D I332E SEA- | 5.3E−08 | 53 | 18.3 | 2.2E+05 | 1.2E−02 |
| mcPEG12(10) | | | | | |
| Anti-HER2 S239D A330L I332E | 3.7E−08 | 37 | | 7.5E+05 | 2.8E−02 |
| Anti-HER2 S239D A330L I332E - | 2.9E−06 | 2900 | 78.4 | 2.9E+04 | 8.5E−02 |
| mcPEG12(10) | | | | | |
| Anti-HER2 S239D A330L I332E SEA | 2.9E−09 | 2.9 | | 9.2E+05 | 2.6E−03 |
| Anti-HER2 S239D A330L I332E | 6.7E−08 | 67 | 23.1 | 1.3E+05 | 8.8E−03 |
| SEA- mcPEG12(10) | | | | | |

TABLE 6
Human F158 FcγRIIIa Biolayer Interferometry

| | K_{D} | K_{D} | K_{D} Fold | kₐ | k_{d} |
|---|---|---|---|---|---|
| Analyte | (M) | (nM) | change | (1/(Ms)) | (1/s) |
| Anti-HER2 | 2.90E−06 | 2900.00 | | 4.20E+04 | 1.20E−01 |
| Anti-HER2-mcPEG12(8) | 4.90E−05 | 49000.00 | 16.9 | 7.80E+03 | 3.80E−01 |
| Anti-HER2 SEA | 1.80E−07 | 180.00 | | 2.90E+05 | 5.20E−02 |
| Anti-HER2 SEA- mcPEG12(8) | 6.60E−06 | 6600.00 | 36.7 | 3.40E+04 | 2.30E−01 |
| Anti-HER2 S239D I332E | 1.10E−07 | 110.00 | | 5.10E+05 | 5.60E−02 |
| Anti-HER2 S239D I332E- | 7.70E−06 | 7700.00 | 70.0 | 1.40E+04 | 1.10E−01 |
| mcPEG12(8) | | | | | |
| Anti-HER2 S239D I332E SEA | 4.40E−09 | 4.40 | | 7.50E+05 | 3.30E−03 |
| Anti-HER2 S239D I332E SEA- | 1.50E−07 | 150.00 | 34.1 | 1.90E+05 | 2.90E−02 |
| mcPEG12(10) | | | | | |
| Anti-HER2 S239D A330L I332E | 9.50E−08 | 95.00 | | 5.20E+05 | 4.90E−02 |
| Anti-HER2 S239D A330L I332E - | 3.80E−06 | 3800.00 | 40.0 | 2.00E+04 | 7.70E−02 |
| mcPEG12(10) | | | | | |
| Anti-HER2 S239D A330L I332E SEA | 2.60E−09 | 2.60 | | 9.00E+05 | 2.40E−03 |
| Anti-HER2 S239D A330L I332E | 1.40E−07 | 140.00 | 53.8 | 1.10E+05 | 1.50E−02 |
| SEA- mcPEG12(10) | | | | | |

TABLE 7
Human FcγRI Biolayer Interferometry

| | K_{D} | K_{D} | K_{D} Fold | kₐ | k_{d} |
|---|---|---|---|---|---|
| Analyte | (M) | (nM) | change | (1/(Ms)) | (1/s) |
| Anti-HER2 | 1.2E−09 | 1.20 | | 4.10E+05 | 5.10E−04 |
| Anti-HER2-mcPEG12(8) | 9.0E−09 | 9.00 | 7.5 | 2.90E+05 | 2.60E−03 |
| Anti-HER2 SEA | 8.9E−10 | 0.89 | | 4.30E+05 | 3.90E−04 |
| Anti-HER2 SEA- mcPEG12(8) | 7.1E−09 | 7.10 | 8.0 | 2.70E+05 | 1.90E−03 |
| Anti-HER2 S239D I332E | 1.0E−10 | 0.10 | | 6.50E+05 | 6.70E−05 |
| Anti-HER2 S239D I332E- | 1.0E−09 | 1.00 | 10.0 | 1.60E+05 | 1.60E−04 |
| mcPEG12(8) | | | | | |
| Anti-HER2 S239D I332E SEA | 1.6E−10 | 0.16 | | 5.50E+05 | 8.90E−05 |
| Anti-HER2 S239D I332E SEA- | 5.2E−10 | 0.52 | 3.3 | 2.90E+05 | 1.50E−04 |
| mcPEG12(10) | | | | | |
| Anti-HER2 S239D A330L I332E | 1.2E−10 | 0.12 | | 5.90E+05 | 6.80E−05 |
| Anti-HER2 S239D A330L I332E - | 1.2E−09 | 1.20 | 10.0 | 9.50E+04 | 1.10E−04 |
| mcPEG12(10) | | | | | |
| Anti-HER2 S239D A330L I332E SEA | 1.1E−10 | 0.11 | | 6.60E+05 | 7.40E−05 |
| Anti-HER2 S239D A330L I332E | 6.4E−10 | 0.64 | 5.8 | 1.60E+05 | 1.00E−04 |
| SEA- mcPEG12(10) | | | | | |

TABLE 8
Human H131 FcγRIIa Biolayer Interferometry

| | K_{D} | K_{D} | K_{D} Fold | kₐ | k_{d} |
|---|---|---|---|---|---|
| Analyte | (M) | (nM) | change | (1/(Ms)) | (1/s) |
| Anti-HER2 | 1.90E−06 | 1900 | | 1.70E+05 | 3.20E−01 |
| Anti-HER2-mcPEG12(8) | 1.30E−05 | 13000 | 6.8 | 3.60E+04 | 4.70E−01 |
| Anti-HER2 SEA | 1.50E−06 | 1500 | | 1.50E+05 | 2.30E−01 |
| Anti-HER2 SEA- mcPEG12(8) | 1.80E−05 | 18000 | 12.0 | 2.70E+04 | 5.00E−01 |
| Anti-HER2 S239D I332E | 1.20E−06 | 1200 | | 2.30E+05 | 2.80E−01 |
| Anti-HER2 S239D I332E- | No Binding | | | | |
| mcPEG12(8) | | | | | |
| Anti-HER2 S239D I332E SEA | 1.70E−06 | 1700 | | 2.40E+05 | 3.90E−01 |
| Anti-HER2 S239D I332E SEA- | No Binding | | | | |
| mcPEG12(10) | | | | | |
| Anti-HER2 S239D A330L I332E | 2.10E−06 | 2100 | | 1.90E+05 | 3.80E−01 |
| Anti-HER2 S239D A330L I332E - | No Binding | | | | |
| mcPEG12(10) | | | | | |
| Anti-HER2 S239D A330L I332E | 3.40E−06 | | | 1.40E+05 | 4.50E−01 |
| SEA | | | | | |
| Anti-HER2 S239D A330L I332E | No Binding | | | | |
| SEA- mcPEG12(10) | | | | | |

### Example 9

### Cynomolgus Macaque Models for PEGylated Antibody Activity

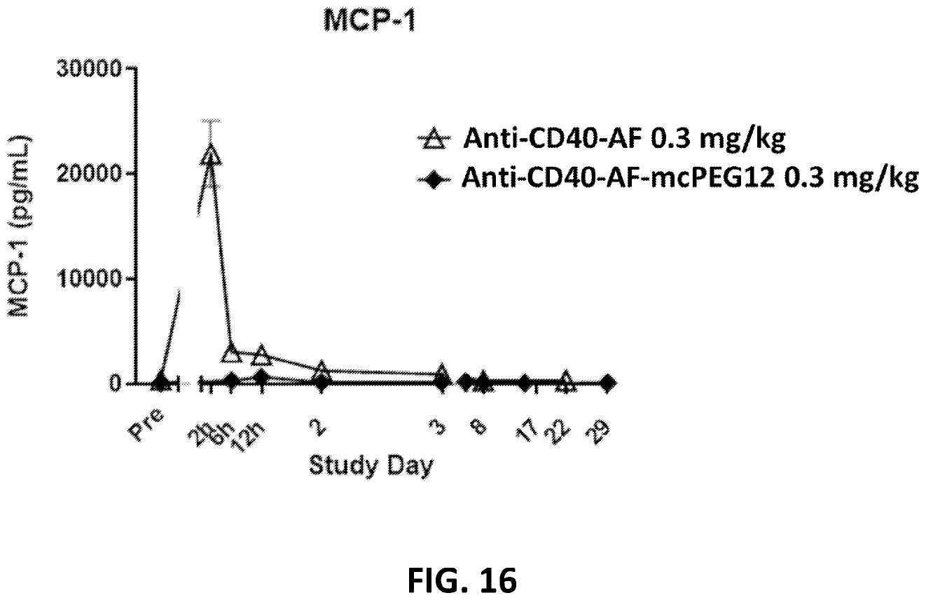

In this example, antibodies and antibody conjugates were administered to cynomolgus macaques via a single bolus intravenous injection at 0.3 mg/kg. The extent of on-target B cell depletion was monitored by flow cytometry of CD20+ lymphocytes in whole blood at the designated timepoints and compared to a pre-dose baseline sample. Cytokine levels were assessed in K2EDTA plasma at the designated timepoints using Luminex multiplexed cytokine analysis and compared to a pre-dose baseline sample. Plasma samples were analyzed for total antibody (TAb) using an ELISA-based immunoassay. Results from these assays are summarized in FIGS. 16-18, with the first day of dosing designated as Day 1.

FIG. 16 summarizes MCP-1 plasma levels prior to (x-axis ‘Pre’) and following non-PEGylated (Anti-CD40-SEA) and PEGylated (Anti-CD40-SEA-MC-PEG12) antibody administration. While non-PEGylated antibody generated a spike in MCP-1 levels (maximizing approximately 2 hours following antibody administration), the PEGylated antibody affected lower minimal MCP-1 increases, with MCP-1 levels maximizing after approximately 12 hours following administration, and reaching less than 4% of the maximum levels affected by the non-PEGylated antibody.

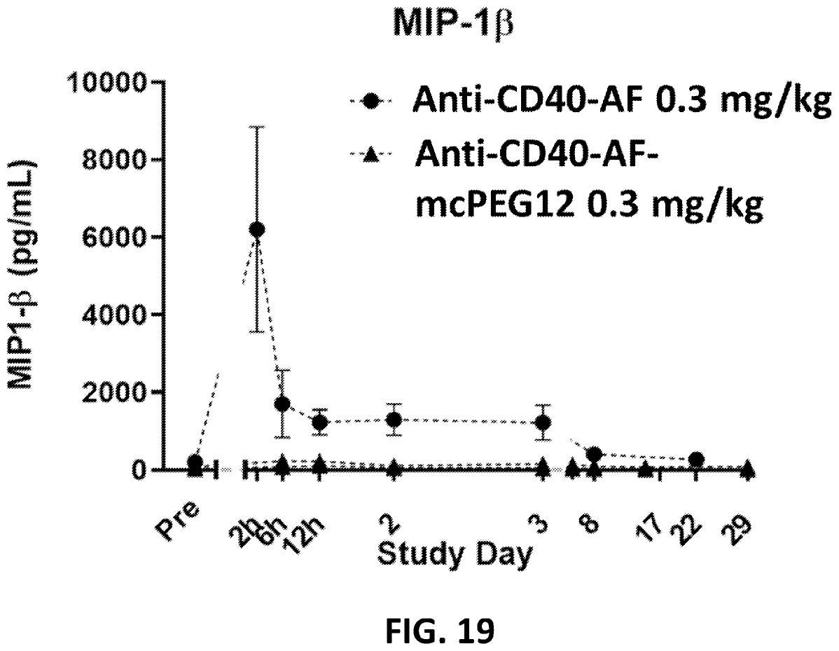

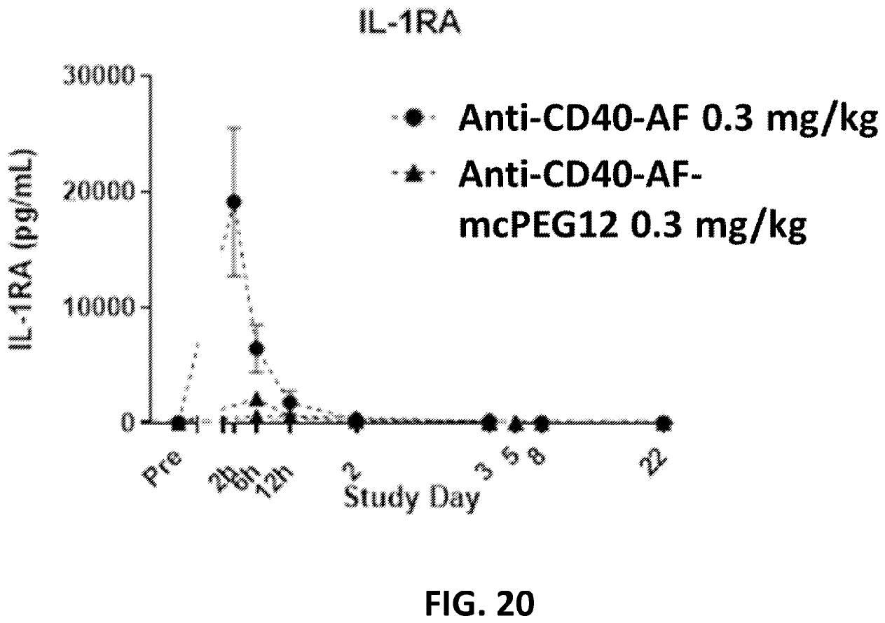

Similar trends were observed for MIP-1β (FIG. 19) and IL-1RA (FIG. 20) plasma levels following 0.3 mg/kg antibody administration. While non-BPM functionalized afucosylated antibody administration lead to ng/mL-level spikes in these cytokines approximately two hours following administration, BPM-functionalized afucosylated antibody administration delayed and suppressed MIP-1β and IL-1RA responses, with maxima for each cytokine not exceeding pg/mL-levels. Peak MCP-1, MIP-1β, and IL-1RA plasma levels following BPM-functionalized and BPM-non-functionalized antibody administration are summarized in TABLE 9.

TABLE 9
Peak Cytokine Levels in Cynomolgus Macaques
Following Antibody Administration

| Cytokine | Anti-CD40-SEA (2 hr) | Anti-CD40-SEA-mcPEG12 (6 hr) |
|---|---|---|
| IL-1RA | 19191 | 1441 |
| MIP-1b | 21950 | 669 |
| MCP-1 | 6200 | 166 |

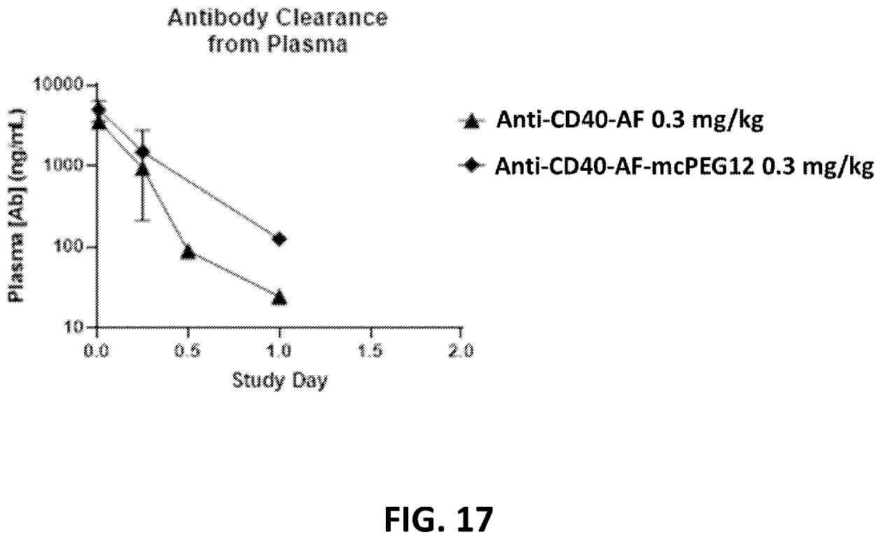

FIG. 17 summarizes antibody levels following administration. The non-PEGylated antibody exhibited greater clearance over the first day following administration than the PEGylated antibody.

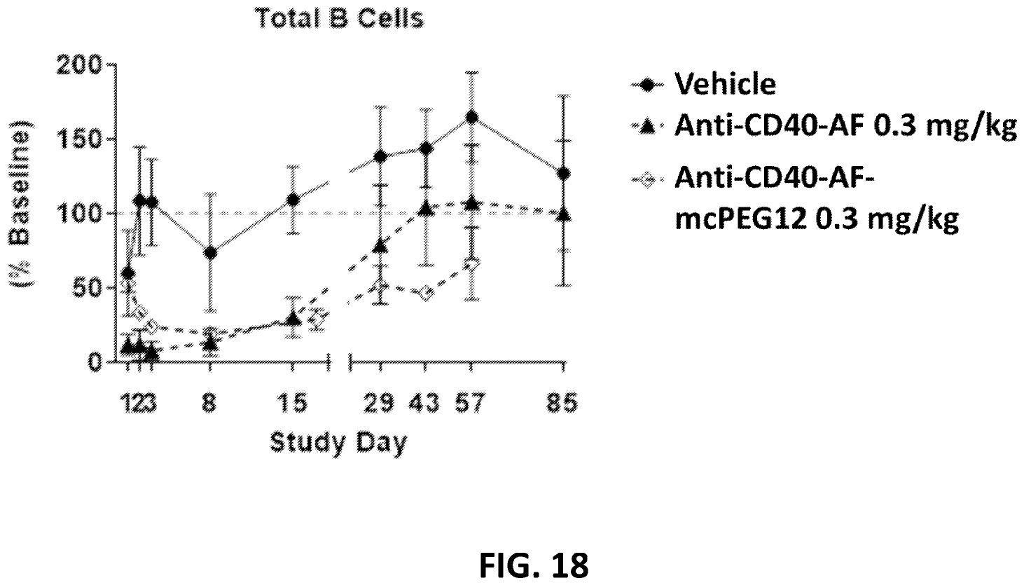

FIG. 18 provides a comparison of B-cell depletion following antibody administration. While both the non-PEGylated and PEGylated antibodies affected approximately 80-90% depletion of B cells at their respective nadirs, the timing of depletion was delayed for the PEGylated antibody, with the nadir occurring at approximately Study Day 8 instead of almost immediate depletion observed for Anti-CD40-SEA.

### Example 10

### Synthesis of an Afucosylated Humanized Anti-CD40 Antibody

A humanized anti-CD40 antibody with heavy and light chains of SEQ ID NOs: 890 and 891, respectively, was expressed in CHO cells. A fucosylation inhibitor, 2-fluorofucose, was included in the cell culture media during the production of antibodies and resulted in non-afucosylation. The base media for cell growth was fucose free and 2-flurofucose was added to the media to inhibit protein fucosylation. Ibid. Incorporation of fucose into antibodies was measured by LC-MS via PLRP-S chromatography and electrospray ionization quadrople TOF MS. Ibid. Data not shown.

1. A modulated effector function (MEF) antibody, wherein the MEF antibody comprises an effector function enhancing modification and an effector function diminishing modification, wherein the effector function diminishing modification comprises a biocompatible polymeric moiety (BPM) with a covalent attachment to an amino acid or post-translational modification of the MEF antibody, wherein the effector function diminishing modification is at least partially reversible.

2. (canceled)

3. The MEF antibody of claim 1, wherein the covalent attachment is cleavable, cleavage of which covalent attachment at least partially reverses the effector function diminishing modification.

4. (canceled)

5. The MEF antibody of claim 1, wherein the effector function enhancing modification increases a binding affinity of the MEF antibody for FcγRI, FcγRIIa, FcγRIIb, FcγRIIIa, FcγRIIIb, or a combination thereof.

6.-7. (canceled)

8. The MEF antibody of claim 1, wherein the amino acid comprises a cysteine residue or a methionine residue.

9.-12. (canceled)

13. The MEF antibody of claim 1, wherein the BPM comprises an enzymatically cleavable moiety.

14.-17. (canceled)

18. The MEF antibody of claim 1, wherein, prior to the at least partial reversal of the effector function diminishing modification, the MEF antibody has between 2% and 20% of an effector function activity of an equivalent antibody lacking the BPM.

19. The MEF antibody of claim 1, wherein the MEF antibody has between 30% and 70% of the effector function activity of an equivalent antibody lacking the BPM following 192 hours incubation in 37° C. human plasma.

20. The MEF antibody of claim 1, wherein, prior to the at least partial reversal of the effector function diminishing modification, the MEF antibody has between 2% and 20% of an FcγRIII binding affinity of an equivalent antibody lacking the BPM.

21. The MEF antibody of claim 1, wherein, 192 hours after administration, the MEF antibody has between 30% and 70% of an FcγRIII binding affinity of an equivalent antibody lacking the BPM.

22.-23. (canceled)

24. A modulated effector function (MEF) antibody coupled to a plurality of biocompatible polymeric moieties (BPM) and an Fc which is at least partially blocked by the BPM; wherein a BPM of the plurality of BPMs is attached to a methionine residue by a cleavable moiety.

25. (canceled)

26. A modulated effector function (MEF) antibody, wherein the MEF antibody has 1, 2, 3, or 4 reduced interchain disulfide bonds and 2, 4, 6, or 8 biocompatible polymeric moieties (BPMs), respectively; wherein each BPM is covalently attached to a sulfur atom of a cysteine residue of a reduced interchain disulfide bond of the MEF antibody via a cleavable moiety; and wherein the MEF antibody exhibits time-dependent reduction in FcR binding, and thus a corresponding time-dependent reduction in an effector function, relative to that of an equivalent antibody.

27. The MEF antibody of claim 26, wherein the MEF antibody has between 2% and 20% of the effector function activity of an equivalent antibody lacking the BPM.

28.-35. (canceled)

36. The MEF antibody of claim 26, wherein each cleavable moiety is covalently attached to a sulfur atom of a cysteine residue of a reduced interchain disulfide bond of the MEF antibody through a cleavable disulfide bond or through a cleavable thioether bond to a non-hydrolyzed succinimide moiety.

37.-39. (canceled)

40. The MEF antibody of claim 26, wherein each cleavable moiety comprises a structure according either to Formula (II) or (III): 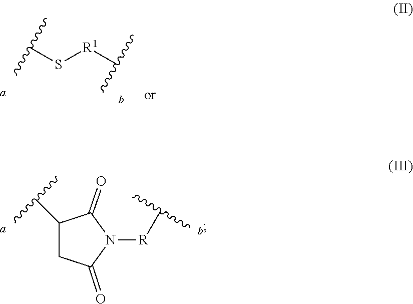
wherein:
R¹ is a C₂-C₁₂ alkylene, optionally interrupted with one of —NH—C(═O)—, —C(═O)NH—, —NH—, and —O—;
R is absent, or is a C₁-C₁₂ alkylene optionally interrupted with one or two of phenyl, —NH—C(═O)—, —C(═O)NH—, —NH—, —O—, —O—C(═O)—, —C(═O)O—, —S—C(═O)—, —C(═O)S—, —O—C(═O)O—, —C(═NR^{1A}), an acetal, —O(SO₂)O—, —O—[P(═O)(—OH)]O—, —C(═N—OH)—, —C(═N—NH₂)—, and —C(R^{1A})═N—NH—; and R is optionally substituted with 1-3 substituents independently selected from phenyl, oxo, and —CO₂R^{A}; C₃-C₆ cycloalkylene; and phenyl optionally substituted with 1-3 independently selected C₁-C₃ alkoxy;
each R^{A} is independently hydrogen or C₁-C₆ alkyl;
each R^{1A} is independently hydrogen or C₁-C₆ alkyl;
wherein [symbol] (a) represents the covalent attachment to the cysteine residue of the reduced interchain disulfide bond of the MEF antibody; and [symbol]
(b) represents the covalent attachment to a BPM or the remainder of the cleavable moiety, which retains covalent attachment to a BPM.

41. The MEF antibody of claim 40, wherein each cleavable moiety has a structure according to Formula (III): 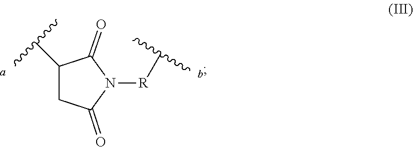
and
wherein R is a C₁-C₁₂ alkylene interrupted with —C(═N—NH₂)— or —C(R^{1A})═N—NH—; or
interrupted with phenyl and one of —C(═N—NH₂)— and —C(R^{1A})═N—NH—.

42. The MEF antibody of claim 40, wherein each cleavable moiety comprises a structure of any one of Formulas (IIa)-(IIi): 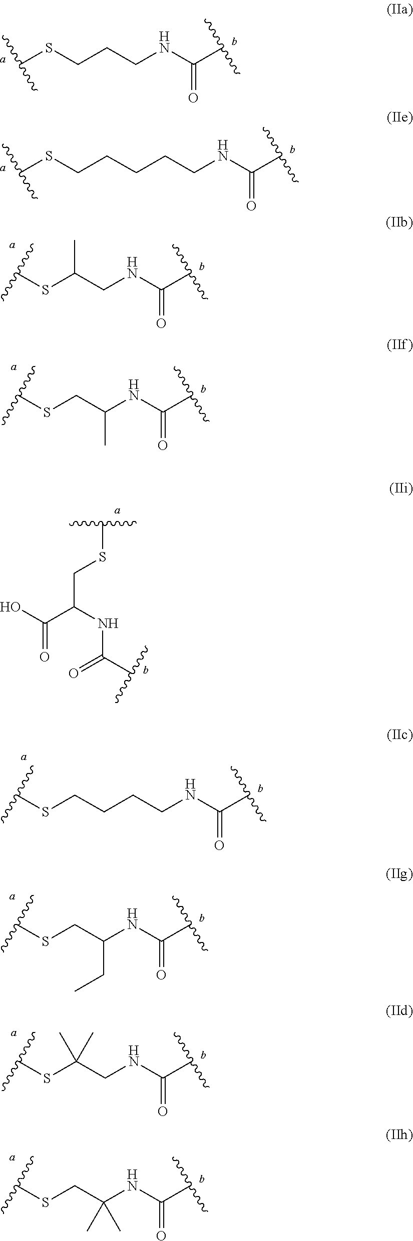
wherein [symbol] (a) represents the covalent attachment to the sulfur atom of a cysteine residue of a reduced interchain disulfide bond of the MEF antibody; and [symbol] (b) represents the covalent attachment of the cleavable moiety to a BPM.

43-48. (canceled)

49. The MEF antibody of claim 26, wherein the time-dependent reduction in FcR binding of the MEF antibody is characterized by an initial reduction in the binding of FcR from at least about 50% to about 90% relative to the equivalent antibody.

50. The MEF antibody of claim 49, wherein the initial reduction in FcR binding of the MEF antibody is characterized by a K_{D} that is about 2-fold to about 1,000-fold higher than the equivalent antibody.

51.-57. (canceled)

58. The MEF antibody of claim 26, wherein each BPM comprises a polyethylene glycol moiety, a polyketal moiety, a polyglycerol moiety, a polysaccharide moiety, a polysarcosine moiety, a polypeptide moiety, or a polyzwitterionic moiety.

59. The MEF antibody of claim 26, wherein the BPMs are monodisperse.

60. (canceled)

61. The MEF antibody of claim 26, wherein the BPMs are polydisperse.

62. (canceled)

63. The MEF antibody of claim 26, wherein each BPM independently has a weight-average molecular weight of about 100 Daltons to about 5,000 Daltons.

64.-69. (canceled)

70. The MEF antibody of claim 26, wherein each BPM comprises a monodispersed PEG2 to PEG72 moiety.

71.-78. (canceled)

79. The MEF antibody of claim 26, each BPM has a structure selected from the group consisting of: 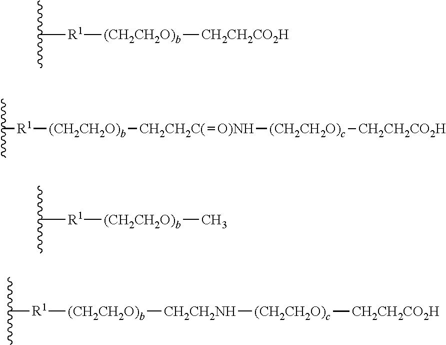
wherein R¹ is a C₂-C₁₂ alkylene, optionally interrupted with one of —NH—C(═O)—, —C(═O)NH—, —NH—, or —O— to which the cleavable moiety is covalently attached, and optionally substituted with —CO₂H;
each subscript b ranges from 2 to 72;
each subscript c ranges from 1 to 72; and [symbol]
indicates site of covalent attachment to the cleavable moiety.

80. The MEF antibody of claim 79, wherein subscript b ranges from 6 to 72 and subscript c ranges from 1-12.

81.-83. (canceled)

84. The MEF antibody of claim 26, wherein each BPM and cleavable moiety, together with a sulfur atom of a cysteine residue of a reduced interchain disulfide bond of the MEF antibody to which the cleavable moiety is covalently attached, has a structure according to any one of Formulas (IIj-IIn) or (IIIa)-(IIIg): 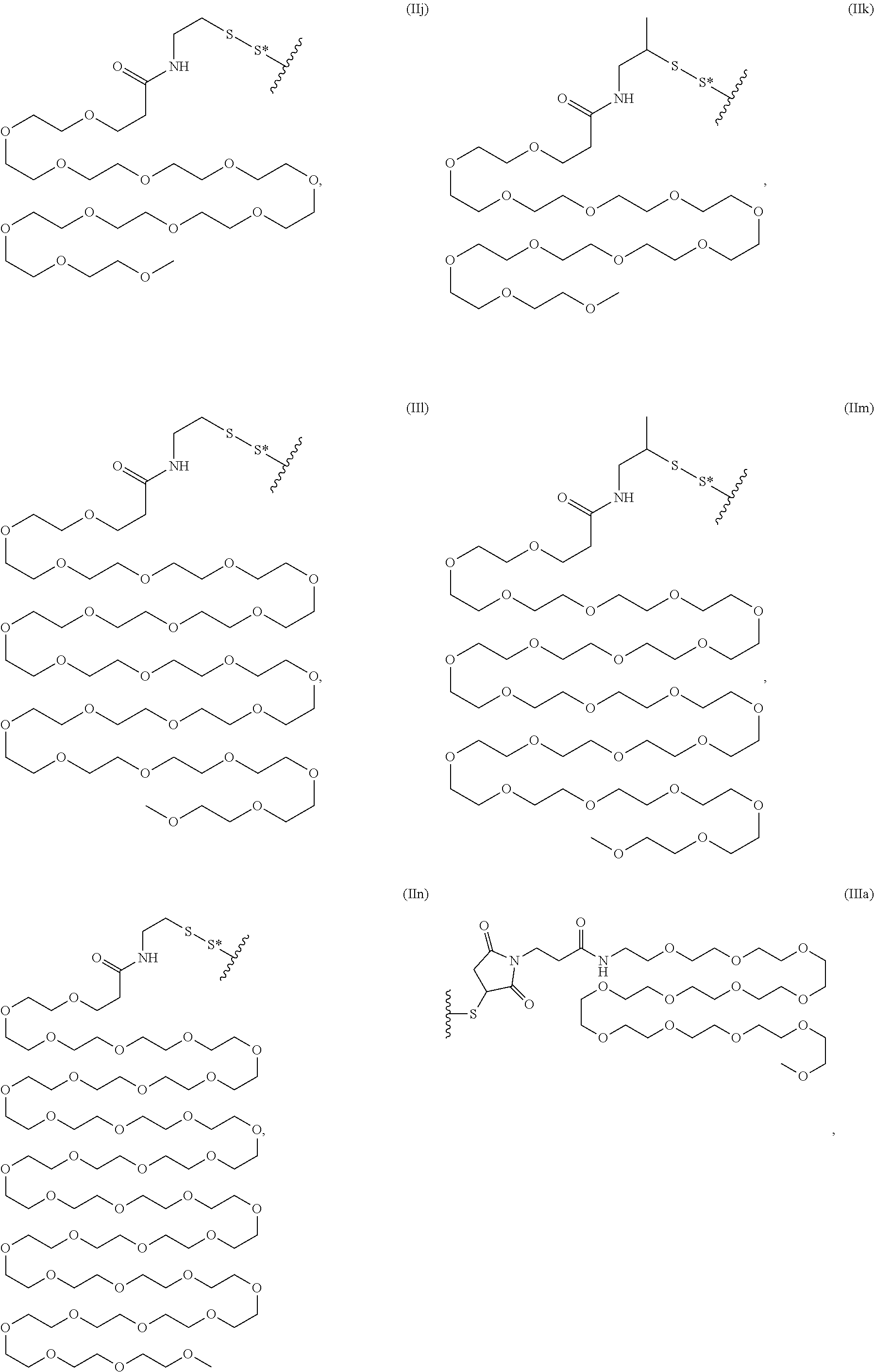 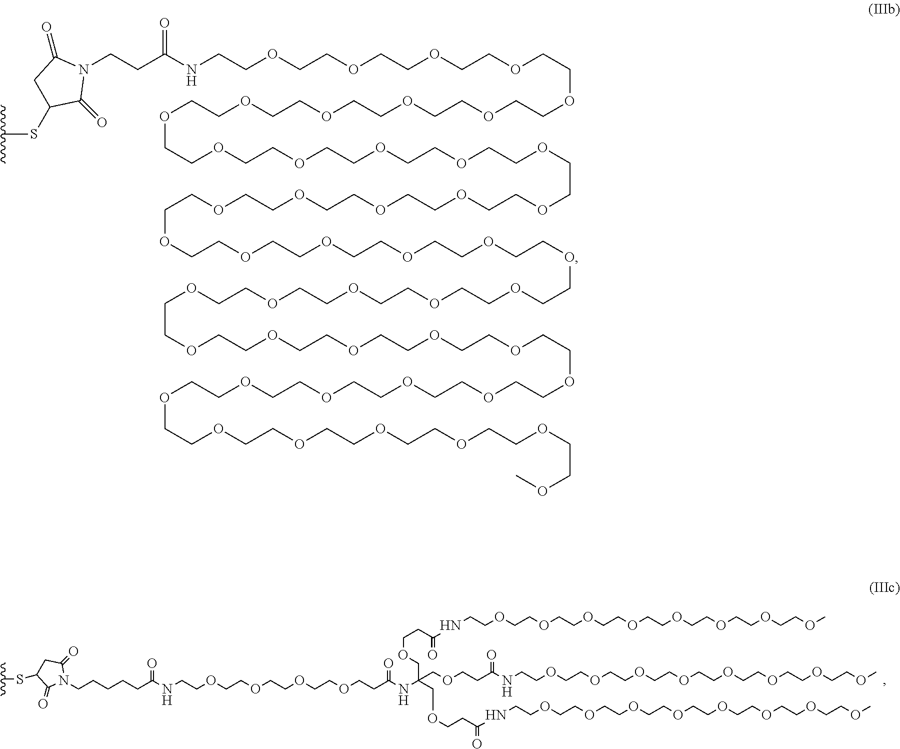 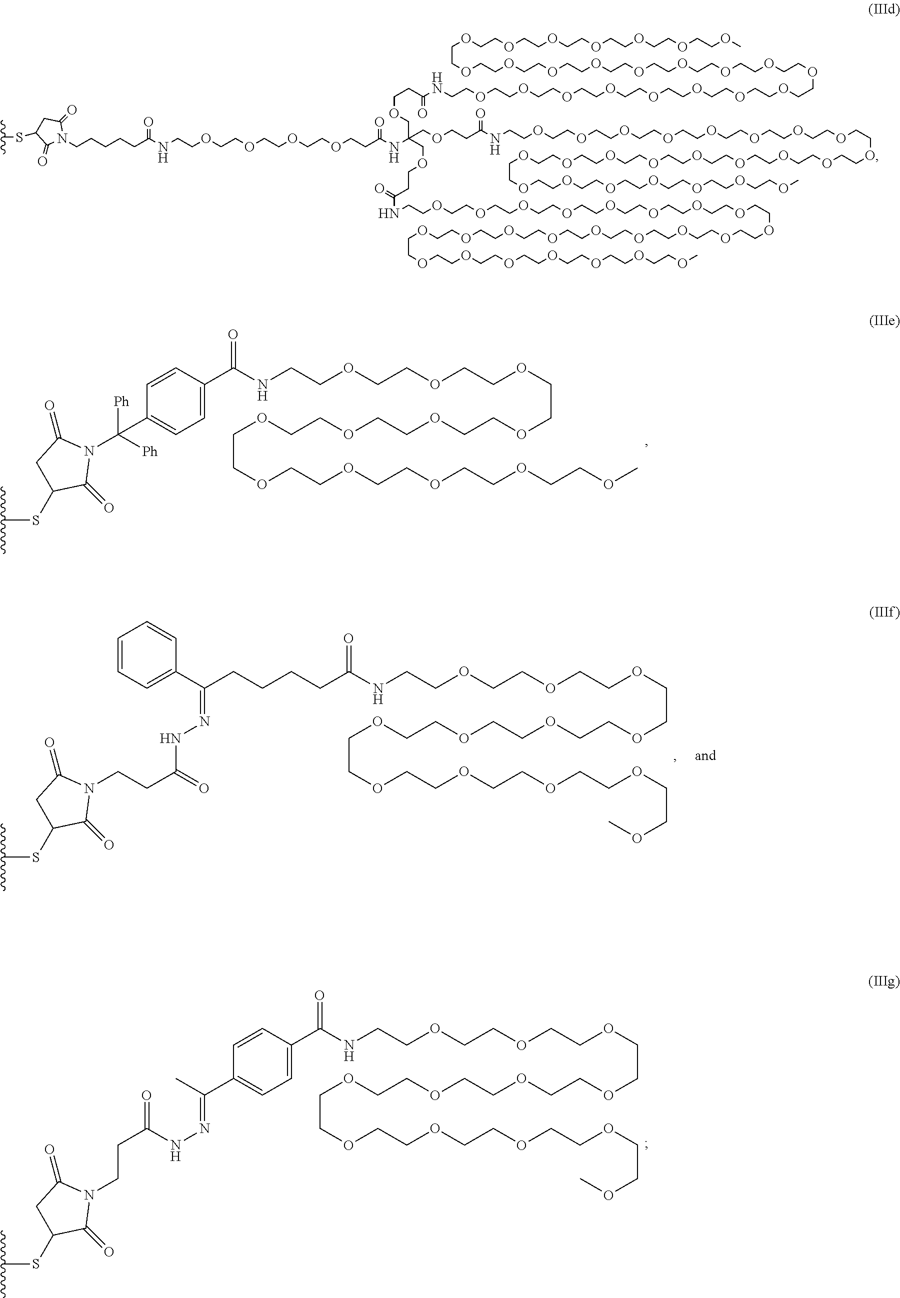
wherein S* is a sulfur atom from a cysteine residue of a reduced interchain disulfide bond of the MEF antibody; and
wherein [symbol] indicates covalent attachment to the remainder of the MEF antibody.

85.-98. (canceled)

99. The MEF antibody of claim 26, wherein the MEF antibody is an IgG₁ antibody, a monoclonal antibody, a chimeric antibody, a humanized antibody, or a combination thereof.

100.-102. (canceled)

103. The MEF antibody of claim 26, wherein the MEF antibody has one or more mutations in the Fc region;
wherein the MEF antibody having the one or more mutations has higher effector function relative to the equivalent antibody.

104. The MEF antibody of claim 103, wherein the MEF antibody is an IgG₁ antibody; and the one or more mutations in the Fc region are selected from the group consisting of S298A, E333A, K334A, S239D, I332E, G236A, S239E, A330L, I332E, G236A, S239D, I332E, G236A, L234Y, G236W, S296A, F243, R292P, Y300L, V305L, and P396L.

105. The MEF antibody of claim 26, wherein the MEF antibody binds to a cancer cell.

106. The MEF antibody of claim 26, wherein the antibody comprises rituximab, obinutuzumab, ofatumumab, trastuzumab, alemtuzumab, mogamulizumab, cetuximab, or dinutuximab.

107. The MEF antibody of claim 26, wherein the MEF antibody binds to an immune cell.

108. The MEF antibody of claim 26, wherein the MEF antibody binds to human CD40.

109. The MEF antibody of claim 26, wherein the MEF antibody comprises a sequence which has at least 80% sequence identity to SEQ ID NO: 890 or SEQ ID NO:891.

110.-130. (canceled)

131. A composition comprising a distribution of MEF antibodies of claim 26.

132.-133. (canceled)

134. The composition of claim 131, further comprising at least one pharmaceutically acceptable carrier.

135.-136. (canceled)

137. The composition of claim 131, wherein the sole active ingredient in the composition is the MEF antibody.

138.-140. (canceled)

141. A method of treating a condition in a subject in need thereof, the method comprising:
administering to the subject a therapeutically effective amount of a composition comprising a modulated effector function (MEF) antibody which comprises an effector function diminishing modification, and which effector function diminishing modification is at least partially reversible under physiological conditions; and
treating the condition while maintaining a systemic level of a cytokine or an inflammatory marker to no more than 10-fold above a level prior to the administering.

142. The method of claim 141, wherein the cytokine or the inflammatory marker is monocyte chemotactic protein-1 (MCP-1), macrophage inflammatory protein-1 (MIP-1β), tumor necrosis factor (TNF-α), interferon gamma (IFN-γ), interleukin-1 receptor agonist (IL-1RA), interleukin 1 beta (IL1B), interleukin 6 (IL6), interleukin 10 (IL10), or a combination thereof.

143. (canceled)

144. The method of claim 141, wherein the modification comprises a cleavable biocompatible polymeric moiety (BPM) covalently attached to an amino acid residue or a post-translational modification of the MEF antibody.

145.-150. (canceled)

151. A method of decreasing the severity, incidence, risk, and/or symptom of an infusion related reaction in a subject associated with an antibody, comprising intravenously administering to the subject a composition comprising the antibody comprising an effector function enhancing modification and an effector function diminishing modification comprising a biocompatible polymeric moiety (BPM) comprising a covalent attachment to an amino acid or post-translational modification of the MEF antibody;
thereby decreasing the severity, incidence, risk, and/or symptom of the infusion related reaction relative to intravenous administration of an equimolar amount of the antibody not comprising the effector function enhancing modification or the effector function diminishing modification.

152.-158. (canceled)

159. The method of claim 151, wherein each cleavable moiety comprises a structure according to either Formula (II) or Formula (III): 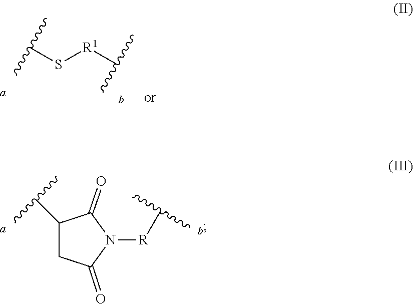
wherein:
R¹ is a C₂-C₁₂ alkylene, optionally interrupted with one of —NH—C(═O)—, —C(═O)NH—, —NH—, and —O—;
R is absent, or is a C₁-C₁₂ alkylene optionally interrupted with one or two of phenyl, —NH—C(═O)—, —C(═O)NH—, —NH—, —O—, —O—C(═O)—, —C(═O)O—, —S—C(═O)—, —C(═O)S—, —O—C(═O)O—, —C(═NR^{1A}), an acetal, —O(SO₂)O—, —O—[P(═O)(—OH)]O—, —C(═N—OH)—, —C(═N—NH₂)—, and —C(R^{1A})═N—NH—; and R is optionally substituted with 1-3 substituents independently selected from phenyl, oxo, and —CO₂R^{A}; C₃-C₆ cycloalkylene; and phenyl optionally substituted with 1-3 independently selected C₁-C₃ alkoxy;
each R^{A} is independently hydrogen or C₁-C₆ alkyl;
each R^{1A} is independently hydrogen or C₁-C₆ alkyl;
wherein [symbol] (a) represents the covalent attachment to the amino acid or the post-translational modification of the MEF antibody; and [symbol]
(b) represents covalent attachment to the remainder of the BPM.

160. (canceled)

161. The method of claim 159, wherein at least about 10% of the BPMs are cleaved from the MEF antibody within about 12 hours and at least about 30% of the BPMs are cleaved from the MEF antibody within 48 hours after intravenous administration.

162.-167. (canceled)

168. A modulated effector function (MEF) antibody having the structure:
Ab-(S*—X-BPM)ₚ
wherein:
each S* is a sulfur atom from a cysteine residue of a reduced interchain disulfide of the MEF antibody;
each X is a cleavable moiety; each BPM is a polyethylene glycol moiety, a polyketal moiety, a polyglycerol moiety, a polysaccharide moiety, a polysarcosine moiety, a polypeptide moiety, or a polyzwitterionic moiety;
subscript p is 2, 4, 6, or 8; and
Ab represents the remainder of the antibody.

169.-171. (canceled)

172. The MEF antibody of claim 168, wherein each X comprises a structure of either Formula (II) or (III): 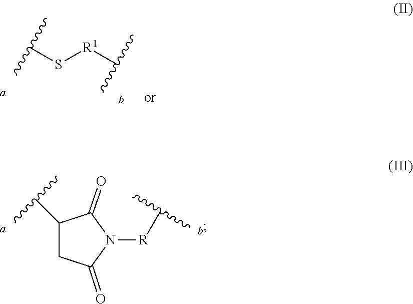
wherein:
R¹ is a C₂-C₁₂ alkylene, optionally interrupted with one of —NH—C(═O)—, —C(═O)NH—, —NH—, and —O—;
R is absent, or is a C₁-C₁₂ alkylene optionally interrupted with one or two of phenyl, —NH—C(═O)—, —C(═O)NH—, —NH—, —O—, —O—C(═O)—, —C(═O)O—, —S—C(═O)—, —C(═O)S—, —O—C(═O)O—, —C(═NR^{1A}), an acetal, —O(SO₂)O—, —O—[P(═O)(—OH)]O—, —C(═N—OH)—, —C(═N—NH₂)—, and —C(R^{1A})═N—NH—; and R is optionally substituted with 1-3 substituents independently selected from phenyl, oxo, and —CO₂R^{A}; C₃-C₆ cycloalkylene; and phenyl optionally substituted with 1-3 independently selected C₁-C₃ alkoxy;
each R^{A} is independently hydrogen or C₁-C₆ alkyl;
each R^{1A} is independently hydrogen or C₁-C₆ alkyl;
wherein [symbol] (a) represents the covalent attachment to the cysteine residue of the reduced interchain disulfide bond of the MEF antibody; and [symbol]
(b) represents the covalent attachment to a BPM, which retains covalent attachment to a BPM.

173.-178. (canceled)

179. A method of treating cancer in a subject comprising administering to the subject a composition comprising an antibody an amino acid or a post-translational modification covalently coupled to a biocompatible polymeric moiety (BPM) through a cleavable linker.


SEQUENCE LISTING (899 sequences)

SEQ ID NO: 1 - protein, 5 aa, synthetic construct
  REGION 1..5: note = Description of Artificial Sequence: Synthetic peptide
DYYIT

SEQ ID NO: 2 - protein, 17 aa, synthetic construct
  REGION 1..17: note = Description of Artificial Sequence: Synthetic peptide
WIYPGSGNTKYNEKFKG

SEQ ID NO: 3 - protein, 8 aa, synthetic construct
  REGION 1..8: note = Description of Artificial Sequence: Synthetic peptide
YGNYWFAY

SEQ ID NO: 4 - protein, 15 aa, synthetic construct
  REGION 1..15: note = Description of Artificial Sequence: Synthetic peptide
KASQSVDFDGDSYMN

SEQ ID NO: 5 - protein, 7 aa, synthetic construct
  REGION 1..7: note = Description of Artificial Sequence: Synthetic peptide
AASNLES

SEQ ID NO: 6 - protein, 9 aa, synthetic construct
  REGION 1..9: note = Description of Artificial Sequence: Synthetic peptide
QQSNEDPWT

SEQ ID NO: 7 - protein, 117 aa, synthetic construct
  REGION 1..117: note = Description of Artificial Sequence: Synthetic polypeptide
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSA

SEQ ID NO: 8 - protein, 111 aa, synthetic construct
  REGION 1..111: note = Description of Artificial Sequence: Synthetic polypeptide
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIK

SEQ ID NO: 9 - protein, 447 aa, synthetic construct
  REGION 1..447: note = Description of Artificial Sequence: Synthetic polypeptide
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 446 aa, synthetic construct
  REGION 1..446: note = Description of Artificial Sequence: Synthetic polypeptide
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 218 aa, synthetic construct
  REGION 1..218: note = Description of Artificial Sequence: Synthetic polypeptide
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 118 aa, synthetic construct
  REGION 1..118: note = Description of Artificial Sequence: Synthetic polypeptide
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 13 - protein, 111 aa, synthetic construct
  REGION 1..111: note = Description of Artificial Sequence: Synthetic polypeptide
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 14 - protein, 448 aa, synthetic construct
  REGION 1..448: note = Description of Artificial Sequence: Synthetic polypeptide
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 218 aa, synthetic construct
  REGION 1..218: note = Description of Artificial Sequence: Synthetic polypeptide
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 5 aa, synthetic construct
  REGION 1..5: note = Description of Artificial Sequence: Synthetic peptide
NYGMN

SEQ ID NO: 17 - protein, 17 aa, synthetic construct
  REGION 1..17: note = Description of Artificial Sequence: Synthetic peptide
WINTYTGEPTYTDDFKG

SEQ ID NO: 18 - protein, 12 aa, synthetic construct
  REGION 1..12: note = Description of Artificial Sequence: Synthetic peptide
GGFGSSYWYFDV

SEQ ID NO: 19 - protein, 11 aa, synthetic construct
  REGION 1..11: note = Description of Artificial Sequence: Synthetic peptide
KASQDVSIAVA

SEQ ID NO: 20 - protein, 7 aa, synthetic construct
  REGION 1..7: note = Description of Artificial Sequence: Synthetic peptide
SASYRYT

SEQ ID NO: 21 - protein, 9 aa, synthetic construct
  REGION 1..9: note = Description of Artificial Sequence: Synthetic peptide
QQHYITPLT

SEQ ID NO: 22 - protein, 121 aa, synthetic construct
  REGION 1..121: note = Description of Artificial Sequence: Synthetic polypeptide
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 23 - protein, 107 aa, synthetic construct
  REGION 1..107: note = Description of Artificial Sequence: Synthetic polypeptide
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIK

SEQ ID NO: 24 - protein, 5 aa, synthetic construct
  REGION 1..5: note = Description of Artificial Sequence: Synthetic peptide
TAGMQ

SEQ ID NO: 25 - protein, 17 aa, synthetic construct
  REGION 1..17: note = Description of Artificial Sequence: Synthetic peptide
WINTHSGVPKYAEDFKG

SEQ ID NO: 26 - protein, 12 aa, synthetic construct
  REGION 1..12: note = Description of Artificial Sequence: Synthetic peptide
SGFGSSYWYFDV

SEQ ID NO: 27 - protein, 11 aa, synthetic construct
  REGION 1..11: note = Description of Artificial Sequence: Synthetic peptide
KASQDVSTAVA

SEQ ID NO: 28 - protein, 7 aa, synthetic construct
  REGION 1..7: note = Description of Artificial Sequence: Synthetic peptide
SASYRYT

SEQ ID NO: 29 - protein, 9 aa, synthetic construct
  REGION 1..9: note = Description of Artificial Sequence: Synthetic peptide
QQHYITPLT

SEQ ID NO: 30 - protein, 121 aa, synthetic construct
  REGION 1..121: note = Description of Artificial Sequence: Synthetic polypeptide
QVQLVQSGAEVKKPGASVKVSCKASGYTFTTAGMQWVRQAPGQGLEWMGWINTHSGVPKYAEDFKGRVTISADTSTSTAYLQLSSLKSEDTAVYYCARSGFGSSYWYFDVWGQGTLVTVSS

SEQ ID NO: 31 - protein, 107 aa, synthetic construct
  REGION 1..107: note = Description of Artificial Sequence: Synthetic polypeptide
DIQMTQSPSSLSASVGDRVTITCKASQDVSTAVAWYQQKPGKAPKLLIYSASYRYTGVPSRFSGSGSGTDFTLTISSLQPEDFAVYYCQQHYITPLTFGQGTKLEIK

SEQ ID NO: 32 - protein, 5 aa, synthetic construct
  REGION 1..5: note = Description of Artificial Sequence: Synthetic peptide
SQNIY

SEQ ID NO: 33 - protein, 17 aa, synthetic construct
  REGION 1..17: note = Description of Artificial Sequence: Synthetic peptide
YIEPYNVVPMYNPKFKG

SEQ ID NO: 34 - protein, 8 aa, synthetic construct
  REGION 1..8: note = Description of Artificial Sequence: Synthetic peptide
SGSSNFDY

SEQ ID NO: 35 - protein, 12 aa, synthetic construct
  REGION 1..12: note = Description of Artificial Sequence: Synthetic peptide
SASSSISSHYLH

SEQ ID NO: 36 - protein, 7 aa, synthetic construct
  REGION 1..7: note = Description of Artificial Sequence: Synthetic peptide
RTSNLAS

SEQ ID NO: 37 - protein, 9 aa, synthetic construct
  REGION 1..9: note = Description of Artificial Sequence: Synthetic peptide
QQGSSLPLT

SEQ ID NO: 38 - protein, 117 aa, synthetic construct
  REGION 1..117: note = Description of Artificial Sequence: Synthetic polypeptide
EIQLVQSGAEVKKPGASVKVSCKASGYAFTSQNIYWVRQAPGQGLEWIGYIEPYNVVPMYNPKFKGRATLTVDKSTSTAYLELSSLRSEDTAVYYCARSGSSNFDYWGQGTLVTVSS

SEQ ID NO: 39 - protein, 108 aa, synthetic construct
  REGION 1..108: note = Description of Artificial Sequence: Synthetic polypeptide
DIQLTQSPSSLSASVGDRVTITCSASSSISSHYLHWYQQKPGKSPKLLIYRTSNLASGVPSRFSGSGSGTDYTLTISSLQPEDFATYYCQQGSSLPLTFGQGTKVEIK

SEQ ID NO: 40 - protein, 5 aa, synthetic construct
  REGION 1..5: note = Description of Artificial Sequence: Synthetic peptide
NYAMH

SEQ ID NO: 41 - protein, 17 aa, synthetic construct
  REGION 1..17: note = Description of Artificial Sequence: Synthetic peptide
LIWYDGSNKFYGDSVKG

SEQ ID NO: 42 - protein, 6 aa, synthetic construct
  REGION 1..6: note = Description of Artificial Sequence: Synthetic peptide
EGSGHY

SEQ ID NO: 43 - protein, 11 aa, synthetic construct
  REGION 1..11: note = Description of Artificial Sequence: Synthetic peptide
RASQGISSALA

SEQ ID NO: 44 - protein, 7 aa, synthetic construct
  REGION 1..7: note = Description of Artificial Sequence: Synthetic peptide
DASSLES

SEQ ID NO: 45 - protein, 9 aa, synthetic construct
  REGION 1..9: note = Description of Artificial Sequence: Synthetic peptide
QQFNSYPIT

SEQ ID NO: 46 - protein, 115 aa, synthetic construct
  REGION 1..115: note = Description of Artificial Sequence: Synthetic polypeptide
QVQLVESGGGVVQPGRSLRLSCAASGFTFSNYAMHWVRQAPGEGLEWVALIWYDGSNKFYGDSVKGRFTISRDNSKNTLYLQMNSLSAEDTAVYYCAREGSGHYWGQGTLVTVSS

SEQ ID NO: 47 - protein, 107 aa, synthetic construct
  REGION 1..107: note = Description of Artificial Sequence: Synthetic polypeptide
AIQLTQSPSSLSASVGDRVTITCRASQGISSALAWYQQKPGKVPKSLIYDASSLESGVPSRFSGSGSGTDFTLTISSLQPEDFATYYCQQFNSYPITFGQGTRLEIK

SEQ ID NO: 48 - protein, 5 aa, synthetic construct
  REGION 1..5: note = Description of Artificial Sequence: Synthetic peptide
NYAMS

SEQ ID NO: 49 - protein, 17 aa, synthetic construct
  REGION 1..17: note = Description of Artificial Sequence: Synthetic peptide
YISPGGDYIYYADSVKG

SEQ ID NO: 50 - protein, 12 aa, synthetic construct
  REGION 1..12: note = Description of Artificial Sequence: Synthetic peptide
DRRHYGSYAMDY

SEQ ID NO: 51 - protein, 16 aa, synthetic construct
  REGION 1..16: note = Description of Artificial Sequence: Synthetic peptide
RSSKSLLHSNLNTYLY

SEQ ID NO: 52 - protein, 7 aa, synthetic construct
  REGION 1..7: note = Description of Artificial Sequence: Synthetic peptide
RMSNLAS

SEQ ID NO: 53 - protein, 9 aa, synthetic construct
  REGION 1..9: note = Description of Artificial Sequence: Synthetic peptide
MQHLEYPFT

SEQ ID NO: 54 - protein, 121 aa, synthetic construct
  REGION 1..121: note = Description of Artificial Sequence: Synthetic polypeptide
QVQLVESGGGLVKPGGSLRLSCAASGFTFSNYAMSWIRQAPGKGLEWVSYISPGGDYIYYADSVKGRFTISRDNAKNSLYLQMNSLRAEDTAVYYCTTDRRHYGSYAMDYWGQGTLVTVSS

SEQ ID NO: 55 - protein, 112 aa, synthetic construct
  REGION 1..112: note = Description of Artificial Sequence: Synthetic polypeptide
DIVMTQSPLSLPVTPGEPASISCRSSKSLLHSNLNTYLYWFLQKPGQSPQILIYRMSNLASGVPDRFSGSGSGTAFTLKISRVEAEDVGVYYCMQHLEYPFTFGPGTKLEIK

SEQ ID NO: 56 - protein, 5 aa, synthetic construct
  REGION 1..5: note = Description of Artificial Sequence: Synthetic peptide
TYAFH

SEQ ID NO: 57 - protein, 17 aa, synthetic construct
  REGION 1..17: note = Description of Artificial Sequence: Synthetic peptide
GIVPIFGTLKYAQKFQD

SEQ ID NO: 58 - protein, 11 aa, synthetic construct
  REGION 1..11: note = Description of Artificial Sequence: Synthetic peptide
AIQLEGRPFDH

SEQ ID NO: 59 - protein, 11 aa, synthetic construct
  REGION 1..11: note = Description of Artificial Sequence: Synthetic peptide
RASQGITSYLA

SEQ ID NO: 60 - protein, 7 aa, synthetic construct
  REGION 1..7: note = Description of Artificial Sequence: Synthetic peptide
AASALQS

SEQ ID NO: 61 - protein, 10 aa, synthetic construct
  REGION 1..10: note = Description of Artificial Sequence: Synthetic peptide
QQVNRGAAIT

SEQ ID NO: 62 - protein, 120 aa, synthetic construct
  REGION 1..120: note = Description of Artificial Sequence: Synthetic polypeptide
QVQLVQSGAEVKKPGSSVRVSCRASGGSSTTYAFHWVRQAPGQGLEWMGGIVPIFGTLKYAQKFQDRVTLTADKSTGTAYMELNSLRLDDTAVYYCARAIQLEGRPFDHWGQGTQVTVSA

SEQ ID NO: 63 - protein, 108 aa, synthetic construct
  REGION 1..108: note = Description of Artificial Sequence: Synthetic polypeptide
DIQLTQSPSFLSASVGDRVTITCRASQGITSYLAWYQQKPGKAPKLLIYAASALQSGVPSRFSGRGSGTEFTLTISSLQPEDFATYYCQQVNRGAAITFGHGTRLDIK

SEQ ID NO: 64 - protein, 5 aa, synthetic construct
  REGION 1..5: note = Description of Artificial Sequence: Synthetic peptide
TYAFH

SEQ ID NO: 65 - protein, 17 aa, synthetic construct
  REGION 1..17: note = Description of Artificial Sequence: Synthetic peptide
GIVPIFGTLKYAQKFQD

SEQ ID NO: 66 - protein, 11 aa, synthetic construct
  REGION 1..11: note = Description of Artificial Sequence: Synthetic peptide
AIQLEGRPFDH

SEQ ID NO: 67 - protein, 11 aa, synthetic construct
  REGION 1..11: note = Description of Artificial Sequence: Synthetic peptide
RASQGITSYLA

SEQ ID NO: 68 - protein, 7 aa, synthetic construct
  REGION 1..7: note = Description of Artificial Sequence: Synthetic peptide
AASALQS

SEQ ID NO: 69 - protein, 10 aa, synthetic construct
  REGION 1..10: note = Description of Artificial Sequence: Synthetic peptide
QQVNRGAAIT

SEQ ID NO: 70 - protein, 120 aa, synthetic construct; identical to SEQ ID NO: 62
  REGION 1..120: note = Description of Artificial Sequence: Synthetic polypeptide

SEQ ID NO: 71 - protein, 108 aa, synthetic construct; identical to SEQ ID NO: 63
  REGION 1..108: note = Description of Artificial Sequence: Synthetic polypeptide

SEQ ID NO: 72 - protein, 5 aa, synthetic construct
  REGION 1..5: note = Description of Artificial Sequence: Synthetic peptide
RYTMH

SEQ ID NO: 73 - protein, 17 aa, synthetic construct
  REGION 1..17: note = Description of Artificial Sequence: Synthetic peptide
VISFDGSNKYYVDSVKG

SEQ ID NO: 74 - protein, 10 aa, synthetic construct
  REGION 1..10: note = Description of Artificial Sequence: Synthetic peptide
EARGSYAFDI

SEQ ID NO: 75 - protein, 11 aa, synthetic construct
  REGION 1..11: note = Description of Artificial Sequence: Synthetic peptide
RASQSVSSYLA

SEQ ID NO: 76 - protein, 7 aa, synthetic construct
  REGION 1..7: note = Description of Artificial Sequence: Synthetic peptide
DASNRAT

SEQ ID NO: 77 - protein, 10 aa, synthetic construct
  REGION 1..10: note = Description of Artificial Sequence: Synthetic peptide
QQRSNWPPFT

SEQ ID NO: 78 - protein, 119 aa, synthetic construct
  REGION 1..119: note = Description of Artificial Sequence: Synthetic polypeptide
QVQLVESGGGVVQPGRSRRLSCAASGFTFSRYTMHWVRQAPGKGLEWVAVISFDGSNKYYVDSVKGRFTISRDNSENTLYLQVNILRAEDTAVYYCAREARGSYAFDIWGQGTMVTVSS

SEQ ID NO: 79 - protein, 108 aa, synthetic construct
  REGION 1..108: note = Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPPFTFGPGTKVDIK

SEQ ID NO: 80 - protein, 5 aa, synthetic construct
  REGION 1..5: note = Description of Artificial Sequence: Synthetic peptide
SFWMH

SEQ ID NO: 81 - protein, 17 aa, synthetic construct
  REGION 1..17: note = Description of Artificial Sequence: Synthetic peptide
YINPRSGYTEYNEIFRD

SEQ ID NO: 82 - protein, 9 aa, synthetic construct
  REGION 1..9: note = Description of Artificial Sequence: Synthetic peptide
FLGRGAMDY

SEQ ID NO: 83 - protein, 11 aa, synthetic construct
  REGION 1..11: note = Description of Artificial Sequence: Synthetic peptide
RASQDISNYLA

SEQ ID NO: 84 - protein, 7 aa, synthetic construct
  REGION 1..7: note = Description of Artificial Sequence: Synthetic peptide
YTSKIHS

SEQ ID NO: 85 - protein, 9 aa, synthetic construct
  REGION 1..9: note = Description of Artificial Sequence: Synthetic peptide
QQGNTFPYT

SEQ ID NO: 86 - protein, 118 aa, synthetic construct [sequence omitted]

SEQ ID NO: 87 - protein, 107 aa, synthetic construct [sequence omitted]

SEQ ID NO: 88 - protein, 7 aa, synthetic construct
  REGION 1..7: note = Description of Artificial Sequence: Synthetic peptide
TSSYYWG

SEQ ID NO: 89 - protein, 16 aa, synthetic construct
  REGION 1..16: note = Description of Artificial Sequence: Synthetic peptide
TIYYNGSTYYSPSLKS

SEQ ID NO: 90 - protein, 11 aa, synthetic construct
  REGION 1..11: note = Description of Artificial Sequence: Synthetic peptide
QGYDIKINIDV

SEQ ID NO: 91 - protein, 11 aa, synthetic construct
  REGION 1..11: note = Description of Artificial Sequence: Synthetic peptide
RASQSVSSYLA

SEQ ID NO: 92 - protein, 7 aa, synthetic construct
  REGION 1..7: note = Description of Artificial Sequence: Synthetic peptide
VASNRAT

SEQ ID NO: 93 - protein, 9 aa, synthetic construct
  REGION 1..9: note = Description of Artificial Sequence: Synthetic peptide
QQRSNWPLT

SEQ ID NO: 94 - protein, 121 aa, synthetic construct
  REGION 1..121: note = Description of Artificial Sequence: Synthetic polypeptide
QLQLQESGPGLVKPSETLSLTCTVSGGSISTSSYYWGWIRQPPGKGLEWIGTIYYNGSTYYSPSLKSRVSISVDTSKNQFSLKLSSVTAADTSVYYCARQGYDIKINIDVWGQGTTVTVSS

SEQ ID NO: 95 - protein, 107 aa, synthetic construct
  REGION 1..107: note = Description of Artificial Sequence: Synthetic polypeptide
EIVLTQSPATLSLSPGERATLSCRASQSVSSYLAWYQQKPGQAPRLLIYVASNRATGIPARFSGSGSGTDFTLTISSLEPEDFAVYYCQQRSNWPLTFGGGTKVEIK

SEQ ID NO: 96 - protein, 5 aa, synthetic construct
  REGION 1..5: note = Description of Artificial Sequence: Synthetic peptide
SSWMN

SEQ ID NO: 97 - protein, 17 aa, synthetic construct
  REGION 1..17: note = Description of Artificial Sequence: Synthetic peptide
RIYPGDGNTHYAQKFQG

SEQ ID NO: 98 - protein, 8 aa, synthetic construct
  REGION 1..8: note = Description of Artificial Sequence: Synthetic peptide
GYLDPMDY

SEQ ID NO: 99 - protein, 11 aa, synthetic construct
  REGION 1..11: note = Description of Artificial Sequence: Synthetic peptide
QASQGINNYLN

SEQ ID NO: 100 - protein, 7 aa, synthetic construct
  REGION 1..7: note = Description of Artificial Sequence: Synthetic peptide
YTSGLHA

SEQ ID NO: 101 - protein, 9 aa, synthetic construct
  REGION 1..9: note = Description of Artificial Sequence: Synthetic peptide
QQYSILPWT

SEQ ID NO: 102 - protein, 117 aa, synthetic construct
  REGION 1..117: note = Description of Artificial Sequence: Synthetic polypeptide
QVQLVQSGAEVKKPGSSVKVSCKASGYAFTSSWMNWVRQAPGQGLEWMGRIYPGDGNTHYAQKFQGRVTLTADKSTSTAYMELSSLRSEDTAVYYCGEGYLDPMDYWGQGTLVTVSS

SEQ ID NO: 103 - protein, 107 aa, synthetic construct
  REGION 1..107: note = Description of Artificial Sequence: Synthetic polypeptide
DIQMTQSPSSLSASVGDRVTITCQASQGINNYLNWYQQKPGKAPKLLIHYTSGLHAGVPSRFSGSGSGTDYTLTISSLEPEDVATYYCQQYSILPWTFGGGTKVEIK

SEQ ID NO: 104 - protein, 5 aa, synthetic construct
  REGION 1..5: note = Description of Artificial Sequence: Synthetic peptide
SYGMH

SEQ ID NO: 105 - protein, 17 aa, synthetic construct
  REGION 1..17: note = Description of Artificial Sequence: Synthetic peptide
VISYDGSNKYYADSVKG

SEQ ID NO: 106 - skipped

SEQ ID NO: 107 - protein, 11 aa, synthetic construct
  REGION 1..11: note = Description of Artificial Sequence: Synthetic peptide
RTSQSISSYLN

SEQ ID NO: 108 - protein, 7 aa, synthetic construct
  REGION 1..7: note = Description of Artificial Sequence: Synthetic peptide
WASTRES

SEQ ID NO: 109 - protein, 9 aa, synthetic construct
  REGION 1..9: note = Description of Artificial Sequence: Synthetic peptide
QQSYDIPYT

SEQ ID NO: 110 - protein, 127 aa, synthetic construct
  REGION 1..127: note = Description of Artificial Sequence: Synthetic polypeptide
EVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKDMGWGSGWRPYYYYGMDVWGQGTTVTVSS

SEQ ID NO: 111 - protein, 107 aa, synthetic construct
  REGION 1..107: note = Description of Artificial Sequence: Synthetic polypeptide
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIK

SEQ ID NO: 112 - protein, 5 aa, synthetic construct
  REGION 1..5: note = Description of Artificial Sequence: Synthetic peptide
NYWMS

SEQ ID NO: 113 - protein, 17 aa, synthetic construct
  REGION 1..17: note = Description of Artificial Sequence: Synthetic peptide
NIKQDGSEKFYADSVKG

SEQ ID NO: 114 - protein, 9 aa, synthetic construct
  REGION 1..9: note = Description of Artificial Sequence: Synthetic peptide
VGPSWEQDY

SEQ ID NO: 115 - protein, 14 aa, synthetic construct
  REGION 1..14: note = Description of Artificial Sequence: Synthetic peptide
TGSSSNIGSYYGVH

SEQ ID NO: 116 - protein, 7 aa, synthetic construct
  REGION 1..7: note = Description of Artificial Sequence: Synthetic peptide
SDTNRPS

SEQ ID NO: 117 - protein, 11 aa, synthetic construct
  REGION 1..11: note = Description of Artificial Sequence: Synthetic peptide
QSYDKGFGHRV

SEQ ID NO: 118 - protein, 118 aa, synthetic construct
  REGION 1..118: note = Description of Artificial Sequence: Synthetic polypeptide
EVQLVESGGGLVQPGGSLRLSCAASGFTFSNYWMSWVRQAPGKGLEWVANIKQDGSEKFYADSVKGRFTISRDNAKNSLYLQMNSLRAEDTAVYYCARVGPSWEQDYWGQGTLVTVSA

SEQ ID NO: 119 - protein, 94 aa, synthetic construct
  REGION 1..94: note = Description of Artificial Sequence: Synthetic polypeptide
QSVLTQPPSVSGAPGQRVTISCTGSSSNIGSYYGVHWYQQLPGTAPKLLIYSDTNRPSGVPDRFSGSKSGTSASLAITGLQAEDEADYYCQSYD

SEQ ID NO: 120 - protein, 5 aa, synthetic construct
  REGION 1..5: note = Description of Artificial Sequence: Synthetic peptide
SYAIS

SEQ ID NO: 121 - protein, 17 aa, synthetic construct
  REGION 1..17: note = Description of Artificial Sequence: Synthetic peptide
GIIPIFGTANYAQKFQG

SEQ ID NO: 122 - protein, 6 aa, synthetic construct
  REGION 1..6: note = Description of Artificial Sequence: Synthetic peptide
GLLWNY

SEQ ID NO: 123 - protein, 11 aa, synthetic construct
  REGION 1..11: note = Description of Artificial Sequence: Synthetic peptide
RASQSVSSNLA

SEQ ID NO: 124 - protein, 7 aa, synthetic construct
  REGION 1..7: note = Description of Artificial Sequence: Synthetic peptide
GASTTAS

SEQ ID NO: 125 - protein, 11 aa, synthetic construct
  REGION 1..11: note = Description of Artificial Sequence: Synthetic peptide
QQYNNWPPAYT

SEQ ID NO: 126 - protein, 115 aa, synthetic construct
  REGION 1..115: note = Description of Artificial Sequence: Synthetic polypeptide
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGLLWNYWGQGTLVTVSS

SEQ ID NO: 127 - protein, 109 aa, synthetic construct
  REGION 1..109: note = Description of Artificial Sequence: Synthetic polypeptide
EIVMTQSPATLSVSPGERATLSCRASQSVSSNLAWYQQKPGQAPRLIIYGASTTASGIPARFSASGSGTDFTLTISSLQSEDFAVYYCQQYNNWPPAYTFGQGTKLEIK

SEQ ID NO: 128 - protein, 5 aa, synthetic construct
  REGION 1..5: note = Description of Artificial Sequence: Synthetic peptide
NYGMN

SEQ ID NO: 129 - protein, 17 aa, synthetic construct
  REGION 1..17: note = Description of Artificial Sequence: Synthetic peptide
WINTYTGEPTYGEDFKG

SEQ ID NO: 130 - protein, 7 aa, synthetic construct
  REGION 1..7: note = Description of Artificial Sequence: Synthetic peptide
FGNYVDY

SEQ ID NO: 131 - protein, 16 aa, synthetic construct
  REGION 1..16: note = Description of Artificial Sequence: Synthetic peptide
RSSKNLLHSNGITYLY

SEQ ID NO: 132 - protein, 7 aa, synthetic construct
  REGION 1..7: note = Description of Artificial Sequence: Synthetic peptide
QMSNLAS

SEQ ID NO: 133 - protein, 9 aa, synthetic construct
  REGION 1..9: note = Description of Artificial Sequence: Synthetic peptide
AQNLEIPRT

SEQ ID NO: 134 - protein, 116 aa, synthetic construct
  REGION 1..116: note = Description of Artificial Sequence: Synthetic polypeptide
QVQLVQSGPEVKKPGASVKVSCKASGYTFTNYGMNWVRQAPGQGLEWMGWINTYTGEPTYGEDFKGRFAFSLDTSASTAYMELSSLRSEDTAVYFCARFGNYVDYWGQGSLVTVSS

SEQ ID NO: 135 - protein, 112 aa, synthetic construct
  REGION 1..112: note = Description of Artificial Sequence: Synthetic polypeptide
DIVMTQSPLSLPVTPGEPASISCRSSKNLLHSNGITYLYWYLQKPGQSPQLLIYQMSNLASGVPDRFSSSGSGTDFTLKISRVEAEDVGVYYCAQNLEIPRTFGQGTKVEIK

SEQ ID NO: 136 - protein, 5 aa, synthetic construct
  REGION 1..5: note = Description of Artificial Sequence: Synthetic peptide
KYGMN

SEQ ID NO: 137 - protein, 17 aa, synthetic construct
  REGION 1..17: note = Description of Artificial Sequence: Synthetic peptide
WINTYTEEPTYGDDFKG

SEQ ID NO: 138 - protein, 7 aa, synthetic construct
  REGION 1..7: note = Description of Artificial Sequence: Synthetic peptide
FGSAVDY

SEQ ID NO: 139 - protein, 16 aa, synthetic construct
  REGION 1..16: note = Description of Artificial Sequence: Synthetic peptide
RSSKSLLHSNGITYLY

SEQ ID NO: 140 - protein, 7 aa, synthetic construct
  REGION 1..7: note = Description of Artificial Sequence: Synthetic peptide
QMSNRAS

SEQ ID NO: 141 - protein, 9 aa, synthetic construct
  REGION 1..9: note = Description of Artificial Sequence: Synthetic peptide
AQNLELPRT

SEQ ID NO: 142 - protein, 116 aa, synthetic construct
  REGION 1..116: note = Description of Artificial Sequence: Synthetic polypeptide
QIQLVQSGPEVKKPGESVKISCKASGYTFTKYGMNWVKQAPGQGLKWMGWINTYTEEPTYGDDFKGRFTFTLDTSTSTAYLEISSLRSEDTATYFCARFGSAVDYWGQGTLVTVSS

SEQ ID NO: 143 - protein, 113 aa, synthetic construct
  REGION 1..113: note = Description of Artificial Sequence: Synthetic polypeptide
DIVMTQSALSNPVTLGESGSISCRSSKSLLHSNGITYLYWYLQKPGQSPQLLIYQMSNRASGVPDRFSSSGSGTDFTLKISRVEAEDVGVYYCAQNLELPRTFGQGTKLEMKR

SEQ ID NO: 144 - protein, 5 aa, synthetic construct
  REGION 1..5: note = Description of Artificial Sequence: Synthetic peptide
DYSMH

SEQ ID NO: 145 - protein, 17 aa, synthetic construct
  REGION 1..17: note = Description of Artificial Sequence: Synthetic peptide
WINTETGEPTYADDFKG

SEQ ID NO: 146 - protein, 4 aa, synthetic construct
  REGION 1..4: note = Description of Artificial Sequence: Synthetic peptide
TAVY

SEQ ID NO: 147 - protein, 11 aa, synthetic construct
  REGION 1..11: note = Description of Artificial Sequence: Synthetic peptide
RASQEISVSLS

SEQ ID NO: 148 - protein, 7 aa, synthetic construct
  REGION 1..7: note = Description of Artificial Sequence: Synthetic peptide
ATSTLDS

SEQ ID NO: 149 - protein, 9 aa, synthetic construct
  REGION 1..9: note = Description of Artificial Sequence: Synthetic peptide
LQYASYPWT

SEQ ID NO: 150 - protein, 113 aa, synthetic construct
  REGION 1..113: note = Description of Artificial Sequence: Synthetic polypeptide
QVKLQESGPELKKPGETVKISCKASGYTFTDYSMHWVKQAPGKGLKWMGWINTETGEPTYADDFKGRFAFSLETSASTAYLQINNLKNEDTATYFCARTAVYWGQGTTVTVSS

SEQ ID NO: 151 - protein, 108 aa, synthetic construct
  REGION 1..108: note = Description of Artificial Sequence: Synthetic polypeptide
DIQMTQSPSSLSASLGERVSLTCRASQEISVSLSWLQQEPDGTIKRLIYATSTLDSGVPKRFSGSRSGSDYSLTISSLESEDFVDYYCLQYASYPWTFGGGTKLEIKR

SEQ ID NO: 152 - protein, 5 aa, synthetic construct
  REGION 1..5: note = Description of Artificial Sequence: Synthetic peptide
NYGMN

SEQ ID NO: 153 - protein, 17 aa, synthetic construct
  REGION 1..17: note = Description of Artificial Sequence: Synthetic peptide
WINTYSGEPRYADDFKG

SEQ ID NO: 154 - protein, 9 aa, synthetic construct
  REGION 1..9: note = Description of Artificial Sequence: Synthetic peptide
DYGRWYFDV

SEQ ID NO: 155 - protein, 10 aa, synthetic construct
  REGION 1..10: note = Description of Artificial Sequence: Synthetic peptide
RASSSVSHMH

SEQ ID NO: 156 - protein, 7 aa, synthetic construct
  REGION 1..7: note = Description of Artificial Sequence: Synthetic peptide
ATSNLAS

SEQ ID NO: 157 - protein, 9 aa, synthetic construct
  REGION 1..9: note = Description of Artificial Sequence: Synthetic peptide
QQWSSTPRT

SEQ ID NO: 158 - protein, 118 aa, synthetic construct
  REGION 1..118: note = Description of Artificial Sequence: Synthetic polypeptide
QIQLVQSGSELKKPGASVKVSCKASGYTFTNYGMNWVRQAPGQDLKWMGWINTYSGEPRYADDFKGRFVFSLDKSVNTAYLQISSLKAEDTAVYYCARDYGRWYFDVWGQGTTVTVSS

SEQ ID NO: 159 - protein, 107 aa, synthetic construct
  REGION 1..107: note = Description of Artificial Sequence: Synthetic polypeptide
QIVLSQSPATLSLSPGERATMSCRASSSVSHMHWYQQKPGQAPRPWIYATSNLASGVPARFSGSGSGTDYTLTISSLEPEDFAVYYCQQWSSTPRTFGGGTKVEIKR

SEQ ID NO: 160 - protein, 5 aa, synthetic construct
  REGION 1..5: note = Description of Artificial Sequence: Synthetic peptide
RYWMS

SEQ ID NO: 161 - protein, 17 aa, synthetic construct
  REGION 1..17: note = Description of Artificial Sequence: Synthetic peptide
EINPDSSTINYAPSLKD

SEQ ID NO: 162 - protein, 10 aa, synthetic construct
  REGION 1..10: note = Description of Artificial Sequence: Synthetic peptide
PDGNYWYFDV

SEQ ID NO: 163 - protein, 11 aa, synthetic construct
  REGION 1..11: note = Description of Artificial Sequence: Synthetic peptide
KASQDVGIAVA

SEQ ID NO: 164 - protein, 7 aa, synthetic construct
  REGION 1..7: note = Description of Artificial Sequence: Synthetic peptide
WASTRHT

SEQ ID NO: 165 - protein, 9 aa, synthetic construct
  REGION 1..9: note = Description of Artificial Sequence: Synthetic peptide
QQYSSYPYT

SEQ ID NO: 166 - protein, 119 aa, synthetic construct
  REGION 1..119: note = Description of Artificial Sequence: Synthetic polypeptide
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSS

SEQ ID NO: 167 - protein, 108 aa, synthetic construct
  REGION 1..108: note = Description of Artificial Sequence: Synthetic polypeptide
DIQMTQSPSSLSASVGDRVTITCKASQDVGIAVAWYQQKPGKVPKLLIYWASTRHTGVPDRFSGSGSGTDFTLTISSLQPEDVATYYCQQYSSYPYTFGQGTKVEIKR

SEQ ID NO: 168 - protein, 5 aa, synthetic construct
  REGION 1..5: note = Description of Artificial Sequence: Synthetic peptide
SFAMS

SEQ ID NO: 169 - protein, 17 aa, synthetic construct
  REGION 1..17: note = Description of Artificial Sequence: Synthetic peptide
AISGSGGGTYYADSVKG

SEQ ID NO: 170 - protein, 13 aa, synthetic construct
  REGION 1..13: note = Description of Artificial Sequence: Synthetic peptide
DKILWFGEPVFDY

SEQ ID NO: 171 - protein, 11 aa, synthetic construct
  REGION 1..11: note = Description of Artificial Sequence: Synthetic peptide
RASQSVSSYLA

SEQ ID NO: 172 - protein, 7 aa, synthetic construct
  REGION 1..7: note = Description of Artificial Sequence: Synthetic peptide
DASNRAT

SEQ ID NO: 173 - protein, 9 aa, synthetic construct
  REGION 1..9: note = Description of Artificial Sequence: Synthetic peptide
QQRSNWPPT

SEQ ID NO: 174 - protein, 122 aa, synthetic construct [sequence omitted]

SEQ ID NO: 175 - protein, 108 aa, synthetic construct
  REGION 1..108: note = Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKR

SEQ ID NO: 176 - protein, 5 aa, synthetic construct
  REGION 1..5: note = Description of Artificial Sequence: Synthetic peptide
SYRMH

SEQ ID NO: 177 - protein, 17 aa, synthetic construct
  REGION 1..17: note = Description of Artificial Sequence: Synthetic peptide
YINPSTGYTEYNQKFKD

SEQ ID NO: 178 - protein, 7 aa, synthetic construct
  REGION 1..7: note = Description of Artificial Sequence: Synthetic peptide
GGGVFDY

SEQ ID NO: 179 - protein, 10 aa, synthetic construct
  REGION 1..10: note = Description of Artificial Sequence: Synthetic peptide
SASSSISYMH

SEQ ID NO: 180 - protein, 7 aa, synthetic construct
  REGION 1..7: note = Description of Artificial Sequence: Synthetic peptide
TTSNLAS

SEQ ID NO: 181 - protein, 9 aa, synthetic construct
  REGION 1..9: note = Description of Artificial Sequence: Synthetic peptide
HQRSTYPLT

SEQ ID NO: 182 - protein, 116 aa, synthetic construct
  REGION 1..116: note = Description of Artificial Sequence: Synthetic polypeptide
QVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSS

SEQ ID NO: 183 - protein, 106 aa, synthetic construct
  REGION 1..106: note = Description of Artificial Sequence: Synthetic polypeptide
DIQMTQSPSTLSASVGDRVTITCSASSSISYMHWYQQKPGKAPKLLIYTTSNLASGVPARFSGSGSGTEFTLTISSLQPDDFATYYCHQRSTYPLTFGQGTKVEVK

SEQ ID NO: 184 - protein, 4 aa, synthetic construct
  REGION 1..4: note = Description of Artificial Sequence: Synthetic peptide
SYWM

SEQ ID NO: 185 - protein, 17 aa, synthetic construct
  REGION 1..17: note = Description of Artificial Sequence: Synthetic peptide
EIIPINGHTNYNEKFKS

SEQ ID NO: 186 - protein, 14 aa, synthetic construct
  REGION 1..14: note = Description of Artificial Sequence: Synthetic peptide
GGYYYYGSRDYFDY

SEQ ID NO: 187 - protein, 15 aa, synthetic construct
  REGION 1..15: note = Description of Artificial Sequence: Synthetic peptide
KASQSVDYDGDSYMN

SEQ ID NO: 188 - protein, 7 aa, synthetic construct
  REGION 1..7: note = Description of Artificial Sequence: Synthetic peptide
AASDLES

SEQ ID NO: 189 - protein, 9 aa, synthetic construct
  REGION 1..9: note = Description of Artificial Sequence: Synthetic peptide
QQSHEDPFT

SEQ ID NO: 190 - protein, 123 aa, synthetic construct
  REGION 1..123: note = Description of Artificial Sequence: Synthetic polypeptide
QVQLQQPGAELVKPGASVKLSCKASGYTFTSYWMHWVKQRPGQGLEWIGEIIPINGHTNYNEKFKSKATLTLDKSSSTAYMQLSSLASEDSAVYYCARGGYYYYGSRDYFDYWGQGTTLTVSS

SEQ ID NO: 191 - protein, 111 aa, synthetic construct
  REGION 1..111: note = Description of Artificial Sequence: Synthetic polypeptide
DIVLTQSPASLAVSLGQRATISCKASQSVDYDGDSYMNWYQQIPGQPPKLLIYAASDLESGIPARFSGSGSGTDFTLNIHPVEEEDAATYYCQQSHEDPFTFGGGTKLEIK

SEQ ID NO: 192 - protein, 4 aa, synthetic construct
  REGION 1..4: note = Description of Artificial Sequence: Synthetic peptide
SYWM

SEQ ID NO: 193 - protein, 17 aa, synthetic construct
  REGION 1..17: note = Description of Artificial Sequence: Synthetic peptide
EIIPIFGHTNYNEKFKS

SEQ ID NO: 194 - protein, 14 aa, synthetic construct
  REGION 1..14: note = Description of Artificial Sequence: Synthetic peptide
GGYYYYPRQGFLDY

SEQ ID NO: 195 - protein, 16 aa, synthetic construct
  REGION 1..16: note = Description of Artificial Sequence: Synthetic peptide
KASQSVDYDSGDSYMN

SEQ ID NO: 196 - protein, 7 aa, synthetic construct
  REGION 1..7: note = Description of Artificial Sequence: Synthetic peptide
AASDLES

SEQ ID NO: 197 - protein, 9 aa, synthetic construct
  REGION 1..9: note = Description of Artificial Sequence: Synthetic peptide
QQSHEDPFT

SEQ ID NO: 198 - protein, 123 aa, synthetic construct
  REGION 1..123: note = Description of Artificial Sequence: Synthetic polypeptide
EVQLVESGGGLVKPGGSLRLSCAASGFTFSSYWMHWVRQAPGKGLEWVGEIIPIFGHTNYNEKFKSRFTISLDNSKNTLYLQMGSLRAEDTAVYYCARGGYYYYPRQGFLDYWGQGTTVTVSS

SEQ ID NO: 199 - protein, 111 aa, synthetic construct
  REGION 1..111: note = Description of Artificial Sequence: Synthetic polypeptide
DIVMTQSPDSLAVSLGERATISCKASQSVDYSGDSYMNWYQQKPGQPPKLLIYAASDLESGIPARFSGSGSGTDFTLTISSLEPEDFATYYCQQSHEDPFTFGQGTKLEIK

SEQ ID NO: 200 - protein, 4 aa, synthetic construct
  REGION 1..4: note = Description of Artificial Sequence: Synthetic peptide
YGMN

SEQ ID NO: 201 - protein, 16 aa, synthetic construct
  REGION 1..16: note = Description of Artificial Sequence: Synthetic peptide
YISSSSSTIYADSVKG

SEQ ID NO: 202 - protein, 6 aa, synthetic construct
  REGION 1..6: note = Description of Artificial Sequence: Synthetic peptide
GPGMDV

SEQ ID NO: 203 - protein, 17 aa, synthetic construct
  REGION 1..17: note = Description of Artificial Sequence: Synthetic peptide
KSSQSVLYSSNNKNYLA

SEQ ID NO: 204 - protein, 7 aa, synthetic construct
  REGION 1..7: note = Description of Artificial Sequence: Synthetic peptide
WASTRES

SEQ ID NO: 205 - protein, 10 aa, synthetic construct
  REGION 1..10: note = Description of Artificial Sequence: Synthetic peptide
QQYYSTPQLT

SEQ ID NO: 206 - protein, 112 aa, synthetic construct
  REGION 1..112: note = Description of Artificial Sequence: Synthetic polypeptide
QVQLQQSGGGVVQPGRSLGLSCAASFTFSSYGMNWVRQAPGKGLEWVSYISSSSSTIYADSVKGRFTISRDNSKNTLYLQMNSLRAEDTAVYYCARGPGMDVWGQGTTVTVS

SEQ ID NO: 207 - protein, 114 aa, synthetic construct
  REGION 1..114: note = Description of Artificial Sequence: Synthetic polypeptide
DIVLTQSPDSLAVSLGERATINCKSSQSVLYSSNNKNYLAWYQQKPGQPPKLLIYWASTRESGVPDRFSGSGSGTDFTPAISSLQAEDVAVYYCQQYYSTPQLTFGGGTKVDIK

SEQ ID NO: 208 - protein, 7 aa, synthetic construct
  REGION 1..7: note = Description of Artificial Sequence: Synthetic peptide
TSGMGVG

SEQ ID NO: 209 - protein, 16 aa, synthetic construct
  REGION 1..16: note = Description of Artificial Sequence: Synthetic peptide
HIWWDDDKRYNPALKS

SEQ ID NO: 210 - protein, 10 aa, synthetic construct
  REGION 1..10: note = Description of Artificial Sequence: Synthetic peptide
MELWSYYFDY

SEQ ID NO: 211 - protein, 10 aa, synthetic construct
  REGION 1..10: note = Description of Artificial Sequence: Synthetic peptide
SASSSVSYMH

SEQ ID NO: 212 - protein, 7 aa, synthetic construct
  REGION 1..7: note = Description of Artificial Sequence: Synthetic peptide
DTSKLAS

SEQ ID NO: 213 - protein, 9 aa, synthetic construct
  REGION 1..9: note = Description of Artificial Sequence: Synthetic peptide
FQGSVYPFT

SEQ ID NO: 214 - protein, 120 aa, synthetic construct
  REGION 1..120: note = Description of Artificial Sequence: Synthetic polypeptide
QVQLQESGPGLVKPSQTLSLTCTVSGGSISTSGMGVGWIRQHPGKGLEWIGHIWWDDDKRYNPALKSRVTISVDTSKNQFSLKLSSVTAADTAVYYCARMELWSYYFDYWGQGTLVTVSS

SEQ ID NO: 215 - protein, 107 aa, synthetic construct
  REGION 1..107: note = Description of Artificial Sequence: Synthetic polypeptide
EIVLTQSPATLSLSPGERATLSCSASSSVSYMHWYQQKPGQAPRLLIYDTSKLASGIPARFSGSGSGTDFTLTISSLEPEDVAVYYCFQGSVYPFTFGQGTKLEIKR

SEQ ID NO: 216 - protein, 5 aa, synthetic construct
  REGION 1..5: note = Description of Artificial Sequence: Synthetic peptide
NYGMN

SEQ ID NO: 217 - protein, 17 aa, synthetic construct
  REGION 1..17: note = Description of Artificial Sequence: Synthetic peptide
WINTYTGEPTYADAFKG

SEQ ID NO: 218 - protein, 9 aa, synthetic construct
  REGION 1..9: note = Description of Artificial Sequence: Synthetic peptide
DYGDYGMDY

SEQ ID NO: 219 - protein, 15 aa, synthetic construct
  REGION 1..15: note = Description of Artificial Sequence: Synthetic peptide
RASKSVSTSGYSFMH

SEQ ID NO: 220 - protein, 7 aa, synthetic construct
  REGION 1..7: note = Description of Artificial Sequence: Synthetic peptide
LASNLES

SEQ ID NO: 221 - protein, 9 aa, synthetic construct
  REGION 1..9: note = Description of Artificial Sequence: Synthetic peptide
QHSREVPWT

SEQ ID NO: 222 - protein, 118 aa, synthetic construct; identical to SEQ ID NO: 12
  REGION 1..118: note = Description of Artificial Sequence: Synthetic polypeptide

SEQ ID NO: 223 - protein, 111 aa, synthetic construct; identical to SEQ ID NO: 13
  REGION 1..111: note = Description of Artificial Sequence: Synthetic polypeptide

SEQ ID NO: 224 - protein, 6 aa, synthetic construct
  REGION 1..6: note = Description of Artificial Sequence: Synthetic peptide
SGYSWH

SEQ ID NO: 225 - protein, 16 aa, synthetic construct
  REGION 1..16: note = Description of Artificial Sequence: Synthetic peptide
YIHSSGSTNYNPSLKS

SEQ ID NO: 226 - protein, 7 aa, synthetic construct
  REGION 1..7: note = Description of Artificial Sequence: Synthetic peptide
YDDYFEY

SEQ ID NO: 227 - protein, 11 aa, synthetic construct
  REGION 1..11: note = Description of Artificial Sequence: Synthetic peptide
KASQNVGFNVA

SEQ ID NO: 228 - protein, 7 aa, synthetic construct
  REGION 1..7: note = Description of Artificial Sequence: Synthetic peptide
SASYRYS

SEQ ID NO: 229 - protein, 9 aa, synthetic construct
  REGION 1..9: note = Description of Artificial Sequence: Synthetic peptide
QQYNWYPFT

SEQ ID NO: 230 - protein, 116 aa, synthetic construct
  REGION 1..116: note = Description of Artificial Sequence: Synthetic polypeptide
EVQLQESGPGLVKPSETLSLTCAVTGYSITSGYSWHWIRQFPGNGLEWMGYIHSSGSTNYNPSLKSRISISRDTSKNQFFLKLSSVTAADTAVYYCAGYDDYFEYWGQGTTVTVSS

SEQ ID NO: 231 - protein, 107 aa, synthetic construct
  REGION 1..107: note = Description of Artificial Sequence: Synthetic polypeptide
DIQMTQSPSSLSASVGDRVTITCKASQNVGFNVAWYQQKPGKSPKALIYSASYRYSGVPSRFSGSGSGTDFTLTISSLQPEDFAEYFCQQYNWYPFTFGQGTKLEIK

SEQ ID NO: 232 - protein, 5 aa, synthetic construct
  REGION 1..5: note = Description of Artificial Sequence: Synthetic peptide
NYWIE

SEQ ID NO: 233 - protein, 17 aa, synthetic construct
  REGION 1..17: note = Description of Artificial Sequence: Synthetic peptide
EILPGTGRTIYNEKFKG

SEQ ID NO: 234 - protein, 13 aa, synthetic construct
  REGION 1..13: note = Description of Artificial Sequence: Synthetic peptide
RDYYGNFYYAMDY

SEQ ID NO: 235 - protein, 11 aa, synthetic construct
  REGION 1..11: note = Description of Artificial Sequence: Synthetic peptide
SASQGINNYLN

SEQ ID NO: 236 - protein, 7 aa, synthetic construct
  REGION 1..7: note = Description of Artificial Sequence: Synthetic peptide
YTSTLQS

SEQ ID NO: 237 - protein, 9 aa, synthetic construct
  REGION 1..9: note = Description of Artificial Sequence: Synthetic peptide
QQYSKLPRT

SEQ ID NO: 238 - protein, 122 aa, synthetic construct
  REGION 1..122: note = Description of Artificial Sequence: Synthetic polypeptide
QVQLQQSGSELMMPGASVKISCKATGYTFSNYWIEWVKQRPGHGLEWIGEILPGTGRTIYNEKFKGKATFTADISSNTVQMQLSSLTSEDSAVYYCARRDYYGNFYYAMDYWGQGTSVTVSS

SEQ ID NO: 239 - protein, 107 aa, synthetic construct
  REGION 1..107: note = Description of Artificial Sequence: Synthetic polypeptide
DIQMTQSTSSLSASLGDRVTISCSASQGINNYLNWYQQKPDGTVELLIYYTSTLQSGVPSRFSGSGSGTDYSLTISNLEPEDIGTYYCQQYSKLPRTFGGGTKLEIK

SEQ ID NO: 240 - protein, 7 aa, synthetic construct
  REGION 1..7: note = Description of Artificial Sequence: Synthetic peptide
TYGMGVG

SEQ ID NO: 241 - protein, 16 aa, synthetic construct
  REGION 1..16: note = Description of Artificial Sequence: Synthetic peptide
NIWWSEDKHYSPSLKS

SEQ ID NO: 242 - protein, 11 aa, synthetic construct
  REGION 1..11: note = Description of Artificial Sequence: Synthetic peptide
IDYGNDYAFTY

SEQ ID NO: 243 - protein, 16 aa, synthetic construct
  REGION 1..16: note = Description of Artificial Sequence: Synthetic peptide
RSSKSLLHSNGITYLY

SEQ ID NO: 244 - protein, 7 aa, synthetic construct
  REGION 1..7: note = Description of Artificial Sequence: Synthetic peptide
QMSNLAS

SEQ ID NO: 245 - protein, 9 aa, synthetic construct
  REGION 1..9: note = Description of Artificial Sequence: Synthetic peptide
AQNLELPYT

SEQ ID NO: 246 - protein, 121 aa, synthetic construct
  REGION 1..121: note = Description of Artificial Sequence: Synthetic polypeptide
QITLKESGPTLVKPTQTLTLTCTFSGFSLSTYGMGVGWIRQPPGKALEWLANIWWSEDKHYSPSLKSRLTITKDTSKNQVVLTITNVDPVDTATYYCVQIDYGNDYAFTYWGQGTLVTVSS

SEQ ID NO: 247 - protein, 112 aa, synthetic construct
  REGION 1..112: note = Description of Artificial Sequence: Synthetic polypeptide
DIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGQGTKLEIK

SEQ ID NO: 248 - protein, 5 aa, synthetic construct
  REGION 1..5: note = Description of Artificial Sequence: Synthetic peptide
RYTMH

SEQ ID NO: 249 - protein, 17 aa, synthetic construct
  REGION 1..17: note = Description of Artificial Sequence: Synthetic peptide
VISFDGSNKYYVDSVKG

SEQ ID NO: 250 - protein, 10 aa, synthetic construct
  REGION 1..10: note = Description of Artificial Sequence: Synthetic peptide
EARGSYAFDI

SEQ ID NO: 251 - protein, 11 aa, synthetic construct
  REGION 1..11: note = Description of Artificial Sequence: Synthetic peptide
RASQSVSSYLA

SEQ ID NO: 252 - protein, 7 aa, synthetic construct
  REGION 1..7: note = Description of Artificial Sequence: Synthetic peptide
DASNRAT

SEQ ID NO: 253 - protein, 10 aa, synthetic construct
  REGION 1..10: note = Description of Artificial Sequence: Synthetic peptide
QQRSNWPPFT

SEQ ID NO: 254 - protein, 119 aa, synthetic construct; identical to SEQ ID NO: 78
  REGION 1..119: note = Description of Artificial Sequence: Synthetic polypeptide

SEQ ID NO: 255 - protein, 108 aa, synthetic construct; identical to SEQ ID NO: 79
  REGION 1..108: note = Description of Artificial Sequence: Synthetic polypeptide

SEQ ID NO: 256 - protein, 5 aa, synthetic construct
  REGION 1..5: note = Description of Artificial Sequence: Synthetic peptide
SFGMH

SEQ ID NO: 257 - protein, 17 aa, synthetic construct
  REGION 1..17: note = Description of Artificial Sequence: Synthetic peptide
YISSGSFTIYYADSVKG

SEQ ID NO: 258 - protein, 9 aa, synthetic construct
  REGION 1..9: note = Description of Artificial Sequence: Synthetic peptide
MRKGYAMDY

SEQ ID NO: 259 - protein, 16 aa, synthetic construct
  REGION 1..16: note = Description of Artificial Sequence: Synthetic peptide
RSSQIIIHSDGNTYLE

SEQ ID NO: 260 - protein, 7 aa, synthetic construct
  REGION 1..7: note = Description of Artificial Sequence: Synthetic peptide
KVSNRFS

SEQ ID NO: 261 - protein, 9 aa, synthetic construct
  REGION 1..9: note = Description of Artificial Sequence: Synthetic peptide
FQGSHVPHT

SEQ ID NO: 262 - protein, 118 aa, synthetic construct
  REGION 1..118: note = Description of Artificial Sequence: Synthetic polypeptide
QVQLVESGGGVVQPGRSLRLSCAASGFTFSSFGMHWVRQAPGKGLEWVAYISSGSFTIYYADSVKGRFTISRDNSKNTLYLQMNSLRAEDTAVYYCARMRKGYAMDYWGQGTLVTVSS

SEQ ID NO: 263 - protein, 112 aa, synthetic construct
  REGION 1..112: note = Description of Artificial Sequence: Synthetic polypeptide
DVVMTQSPLSLPVTLGQPASISCRSSQIIIHSDGNTYLEWFQQRPGQSPRRLIYKVSNRFSGVPDRFSGSGSGTDFTLKISRVEAEDVGVYYCFQGSHVPHTFGQGTKVEIK

SEQ ID NO: 264 - protein, 5 aa, synthetic construct
  REGION 1..5: note = Description of Artificial Sequence: Synthetic peptide
NYGVN

SEQ ID NO: 265 - protein, 17 aa, synthetic construct
  REGION 1..17: note = Description of Artificial Sequence: Synthetic peptide
WINPNTGEPTFDDDFKG

SEQ ID NO: 266 - protein, 11 aa, synthetic construct
  REGION 1..11: note = Description of Artificial Sequence: Synthetic peptide
SRGKNEAWFAY

SEQ ID NO: 267 - protein, 16 aa, synthetic construct
  REGION 1..16: note = Description of Artificial Sequence: Synthetic peptide
RSSQSLVHRNGNTYLH

SEQ ID NO: 268 - protein, 7 aa, synthetic construct
  REGION 1..7: note = Description of Artificial Sequence: Synthetic peptide
TVSNRFS

SEQ ID NO: 269 - protein, 9 aa, synthetic construct
  REGION 1..9: note = Description of Artificial Sequence: Synthetic peptide
SQSSHVPPT

SEQ ID NO: 270 - protein, 120 aa, synthetic construct
  REGION 1..120: note = Description of Artificial Sequence: Synthetic polypeptide
QVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSS

SEQ ID NO: 271 - protein, 112 aa, synthetic construct
  REGION 1..112: note = Description of Artificial Sequence: Synthetic polypeptide
DIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIK

SEQ ID NO: 272 - protein, 5 aa, synthetic construct
  REGION 1..5: note = Description of Artificial Sequence: Synthetic peptide
TYWMS

SEQ ID NO: 273 - protein, 17 aa, synthetic construct
  REGION 1..17: note = Description of Artificial Sequence: Synthetic peptide
EIHPDSSTINYAPSLKD

SEQ ID NO: 274 - protein, 10 aa, synthetic construct
  REGION 1..10: note = Description of Artificial Sequence: Synthetic peptide
LYFGFPWFAY

SEQ ID NO: 275 - protein, 11 aa, synthetic construct
  REGION 1..11: note = Description of Artificial Sequence: Synthetic peptide
KASQDVGTSVA

SEQ ID NO: 276 - protein, 7 aa, synthetic construct
  REGION 1..7: note = Description of Artificial Sequence: Synthetic peptide
WTSTRHT

SEQ ID NO: 277 - protein, 8 aa, synthetic construct
  REGION 1..8: note = Description of Artificial Sequence: Synthetic peptide
QQYSLYRS

SEQ ID NO: 278 - protein, 119 aa, synthetic construct
  REGION 1..119: note = Description of Artificial Sequence: Synthetic polypeptide
EVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSS

SEQ ID NO: 279 - protein, 106 aa, synthetic construct
  REGION 1..106: note = Description of Artificial Sequence: Synthetic polypeptide
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIK

SEQ ID NO: 280 - protein, 5 aa, synthetic construct
  REGION 1..5: note = Description of Artificial Sequence: Synthetic peptide
YYGMN

SEQ ID NO: 281 - protein, 17 aa, synthetic construct
  REGION 1..17: note = Description of Artificial Sequence: Synthetic peptide
WIDTTTGEPTYAQKFQG

SEQ ID NO: 282 - protein, 10 aa, synthetic construct
  REGION 1..10: note = Description of Artificial Sequence: Synthetic peptide
RGPYNWYFDV

SEQ ID NO: 283 - protein, 16 aa, synthetic construct
  REGION 1..16: note = Description of Artificial Sequence: Synthetic peptide
RSSKSLLHSNGNTYLY

SEQ ID NO: 284 - protein, 7 aa, synthetic construct
  REGION 1..7: note = Description of Artificial Sequence: Synthetic peptide
RMSNLVS

SEQ ID NO: 285 - protein, 9 aa, synthetic construct
  REGION 1..9: note = Description of Artificial Sequence: Synthetic peptide
LQHLEYPFT

SEQ ID NO: 286 - protein, 119 aa, synthetic construct
  REGION 1..119: note = Description of Artificial Sequence: Synthetic polypeptide
QVQLVQSGAEVKKPGETVKISCKASDYTFTYYGMNWVKQAPGQGLKWMGWIDTTTGEPTYAQKFQGRIAFSLETSASTAYLQIKSLKSEDTATYFCARRGPYNWYFDVWGQGTTVTVSS

SEQ ID NO: 287 - protein, 112 aa, synthetic construct
  REGION 1..112: note = Description of Artificial Sequence: Synthetic polypeptide
DIVMTQSPLSVPVTPGEPVSISCRSSKSLLHSNGNTYLYWFLQRPGQSPQLLIYRMSNLVSGVPDRFSGSGSGTAFTLRISRVEAEDVGVYYCLQHLEYPFTFGPGTKLELK

SEQ ID NO: 288 - protein, 5 aa, synthetic construct
  REGION 1..5: note = Description of Artificial Sequence: Synthetic peptide
NYGMN

SEQ ID NO: 289 - protein, 17 aa, synthetic construct
  REGION 1..17: note = Description of Artificial Sequence: Synthetic peptide
WINTYTGEPTYADDFKG

SEQ ID NO: 290 - protein, 9 aa, synthetic construct
  REGION 1..9: note = Description of Artificial Sequence: Synthetic peptide
IGDSSPSDY

SEQ ID NO: 291 - protein, 11 aa, synthetic construct
  REGION 1..11: note = Description of Artificial Sequence: Synthetic peptide
KASQSVSNDVV

SEQ ID NO: 292 - protein, 7 aa, synthetic construct
  REGION 1..7: note = Description of Artificial Sequence: Synthetic peptide
YASNRYT

SEQ ID NO: 293 - protein, 9 aa, synthetic construct
  REGION 1..9: note = Description of Artificial Sequence: Synthetic peptide
QQDYTSPWT

SEQ ID NO: 294 - protein, 118 aa, synthetic construct
  REGION 1..118: note = Description of Artificial Sequence: Synthetic polypeptide
QVQLVQSGAEVKKPGASVKVSCKASGYTFTNYGMNWVRQAPGQGLEWMGWINTYTGEPTYADDFKGRVTMTTDTSTSTAYMELRSLRSDDTAVYYCARIGDSSPSDYWGQGTLVTVSS

SEQ ID NO: 295 - protein, 107 aa, synthetic construct
  REGION 1..107: note = Description of Artificial Sequence: Synthetic polypeptide
EIVMTQSPATLSVSPGERATLSCKASQSVSNDVVWYQQKPGQAPRLLIYYASNRYTGIPARFSGSGSGTEFTLTISSLQSEDFAVYYCQQDYTSPWTFGQGTKLEIK

SEQ ID NO: 296 - protein, 5 aa, synthetic construct
  REGION 1..5: note = Description of Artificial Sequence: Synthetic peptide
SYWIE

SEQ ID NO: 297 - protein, 17 aa, synthetic construct
  REGION 1..17: note = Description of Artificial Sequence: Synthetic peptide
EILPGSGNTYYNERFKD

SEQ ID NO: 298 - protein, 14 aa, synthetic construct
  REGION 1..14: note = Description of Artificial Sequence: Synthetic peptide
RAAAYYSNPEWFAY

SEQ ID NO: 299 - protein, 12 aa, synthetic construct
  REGION 1..12: note = Description of Artificial Sequence: Synthetic peptide
TASSSVNSFYLH

SEQ ID NO: 300 - protein, 7 aa, synthetic construct
  REGION 1..7: note = Description of Artificial Sequence: Synthetic peptide
STSNLAS

SEQ ID NO: 301 - protein, 9 aa, synthetic construct
  REGION 1..9: note = Description of Artificial Sequence: Synthetic peptide
HQYHRSPYT

SEQ ID NO: 302 - protein, 123 aa, synthetic construct
  REGION 1..123: note = Description of Artificial Sequence: Synthetic polypeptide
QVQLVQSGAEVKKPGSSVKVSCKASGGTFSSYWIEWVRQAPGQGLEWMGEILPGSGNTYYNERFKDRVTITADESTSTAYMELSSLRSEDTAVYYCARRAAAYYSNPEWFAYWGQGTLVTVSS

SEQ ID NO: 303 - protein, 108 aa, synthetic construct
  REGION 1..108: note = Description of Artificial Sequence: Synthetic polypeptide
EIVLTQSPATLSLSPGERATLSCTASSSVNSFYLHWYQQKPGLAPRLLIYSTSNLASGIPDRFSGSGSGTDFTLTISRLEPEDFAVYYCHQYHRSPYTFGQGTKLEIK

SEQ ID NO: 304 - protein, 5 aa, synthetic construct
  REGION 1..5: note = Description of Artificial Sequence: Synthetic peptide
SYWMQ

SEQ ID NO: 305 - protein, 17 aa, synthetic construct
  REGION 1..17: note = Description of Artificial Sequence: Synthetic peptide
TIYPGDGDTTYTQKFQG

SEQ ID NO: 306 - protein, 10 aa, synthetic construct
  REGION 1..10: note = Description of Artificial Sequence: Synthetic peptide
YDAPGYAMDY

SEQ ID NO: 307 - protein, 11 aa, synthetic construct
  REGION 1..11: note = Description of Artificial Sequence: Synthetic peptide
RASQDINNYLA

SEQ ID NO: 308 - protein, 7 aa, synthetic construct
  REGION 1..7: note = Description of Artificial Sequence: Synthetic peptide
YTSTLHP

SEQ ID NO: 309 - protein, 9 aa, synthetic construct
  REGION 1..9: note = Description of Artificial Sequence: Synthetic peptide
LQYDNLLYT

SEQ ID NO: 310 - protein, 119 aa, synthetic construct
  REGION 1..119: note = Description of Artificial Sequence: Synthetic polypeptide
QVQLVQSGAEVAKPGASVKLSCKASGYTFTSYWMQWVKQRPGQGLECIGTIYPGDGDTTYTQKFQGKATLTADKSSSTAYMQLSSLRSEDSAVYYCARYDAPGYAMDYWGQGTLVTVSS

SEQ ID NO: 311 - protein, 107 aa, synthetic construct
  REGION 1..107: note = Description of Artificial Sequence: Synthetic polypeptide
DIQMTQSPSSLSASVGDRVTITCRASQDINNYLAWYQHKPGKGPKLLIHYTSTLHPGIPSRFSGSGSGRDYSFSISSLEPEDIATYYCLQYDNLLYTFGQGTKLEIK

SEQ ID NO: 312 - protein, 6 aa, synthetic construct
  REGION 1..6: note = Description of Artificial Sequence: Synthetic peptide
RDFAWN

SEQ ID NO: 313 - protein, 16 aa, synthetic construct
  REGION 1..16: note = Description of Artificial Sequence: Synthetic peptide
YISYNGNTRYQPSLKS

SEQ ID NO: 314 - protein, 7 aa, synthetic construct
  REGION 1..7: note = Description of Artificial Sequence: Synthetic peptide
ASRGFPY

SEQ ID NO: 315 - protein, 11 aa, synthetic construct
  REGION 1..11: note = Description of Artificial Sequence: Synthetic peptide
HSSQDINSNIG

SEQ ID NO: 316 - protein, 7 aa, synthetic construct
  REGION 1..7: note = Description of Artificial Sequence: Synthetic peptide
HGTNLDD

SEQ ID NO: 317 - protein, 9 aa, synthetic construct
  REGION 1..9: note = Description of Artificial Sequence: Synthetic peptide
VQYAQFPWT

SEQ ID NO: 318 - protein, 116 aa, synthetic construct
  REGION 1..116: note = Description of Artificial Sequence: Synthetic polypeptide
EVQLQESGPGLVKPSQTLSLTCTVSGYSISRDFAWNWIRQPPGKGLEWMGYISYNGNTRYQPSLKSRITISRDTSKNQFFLKLNSVTAADTATYYCVTASRGFPYWGQGTLVTVSS

SEQ ID NO: 319 - protein, 107 aa, synthetic construct
  REGION 1..107: note = Description of Artificial Sequence: Synthetic polypeptide
DIQMTQSPSSMSVSVGDRVTITCHSSQDINSNIGWLQQKPGKSFKGLIYHGTNLDDGVPSRFSGSGSGTDYTLTISSLQPEDFATYYCVQYAQFPWTFGGGTKLEIK

SEQ ID NO: 320 - protein, 6 aa, synthetic construct
  REGION 1..6: note = Description of Artificial Sequence: Synthetic peptide
RDFAWN

SEQ ID NO: 321 - protein, 16 aa, synthetic construct
  REGION 1..16: note = Description of Artificial Sequence: Synthetic peptide
YISYNGNTRYQPSLKS

SEQ ID NO: 322 - protein, 7 aa, synthetic construct
  REGION 1..7: note = Description of Artificial Sequence: Synthetic peptide
ASRGFPY

SEQ ID NO: 323 - protein, 11 aa, synthetic construct
  REGION 1..11: note = Description of Artificial Sequence: Synthetic peptide
HSSQDINSNIG

SEQ ID NO: 324 - protein, 7 aa, synthetic construct
  REGION 1..7: note = Description of Artificial Sequence: Synthetic peptide
HGTNLDD

SEQ ID NO: 325 - protein, 9 aa, synthetic construct
  REGION 1..9: note = Description of Artificial Sequence: Synthetic peptide
VQYAQFPWT

SEQ ID NO: 326 - protein, 116 aa, synthetic construct; identical to SEQ ID NO: 318
  REGION 1..116: note = Description of Artificial Sequence: Synthetic polypeptide

SEQ ID NO: 327 - protein, 107 aa, synthetic construct; identical to SEQ ID NO: 319
  REGION 1..107: note = Description of Artificial Sequence: Synthetic polypeptide

SEQ ID NO: 328 - protein, 5 aa, synthetic construct
  REGION 1..5: note = Description of Artificial Sequence: Synthetic peptide
NYGVH

SEQ ID NO: 329 - protein, 16 aa, synthetic construct
  REGION 1..16: note = Description of Artificial Sequence: Synthetic peptide
VIWSGGNTDYNTPFTS

SEQ ID NO: 330 - protein, 11 aa, synthetic construct
  REGION 1..11: note = Description of Artificial Sequence: Synthetic peptide
ALTYYDYEFAY

SEQ ID NO: 331 - protein, 11 aa, synthetic construct
  REGION 1..11: note = Description of Artificial Sequence: Synthetic peptide
RASQSIGTNIH

SEQ ID NO: 332 - protein, 7 aa, synthetic construct
  REGION 1..7: note = Description of Artificial Sequence: Synthetic peptide
YASESIS

SEQ ID NO: 333 - protein, 9 aa, synthetic construct
  REGION 1..9: note = Description of Artificial Sequence: Synthetic peptide
QQNNNWPTT

SEQ ID NO: 334 - protein, 119 aa, synthetic construct
  REGION 1..119: note = Description of Artificial Sequence: Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 335 - protein, 107 aa, synthetic construct
  REGION 1..107: note = Description of Artificial Sequence: Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 336 - protein, 5 aa, synthetic construct
  REGION 1..5: note = Description of Artificial Sequence: Synthetic peptide
GYFMN

SEQ ID NO: 337 - protein, 17 aa, synthetic construct
  REGION 1..17: note = Description of Artificial Sequence: Synthetic peptide
RIHPYDGDTFYNQKFQG

SEQ ID NO: 338 - protein, 9 aa, synthetic construct
  REGION 1..9: note = Description of Artificial Sequence: Synthetic peptide
YDGSRAMDY

SEQ ID NO: 339 - protein, 15 aa, synthetic construct
  REGION 1..15: note = Description of Artificial Sequence: Synthetic peptide
KASQSVSFAGTSLMH

SEQ ID NO: 340 - protein, 7 aa, synthetic construct
  REGION 1..7: note = Description of Artificial Sequence: Synthetic peptide
RASNLEA

SEQ ID NO: 341 - protein, 9 aa, synthetic construct
  REGION 1..9: note = Description of Artificial Sequence: Synthetic peptide
QQSREYPYT

SEQ ID NO: 342 - protein, 118 aa, synthetic construct
  REGION 1..118: note = Description of Artificial Sequence: Synthetic polypeptide
QVQLVQSGAEVVKPGASVKISCKASGYTFTGYFMNWVKQSPGQSLEWIGRIHPYDGDTFYNQKFQGKATLTVDKSSNTAHMELLSLTSEDFAVYYCTRYDGSRAMDYWGQGTTVTVSS

SEQ ID NO: 343 - protein, 111 aa, synthetic construct
  REGION 1..111: note = Description of Artificial Sequence: Synthetic polypeptide
DIVLTQSPLSLAVSLGQPAIISCKASQSVSFAGTSLMHWYHQKPGQQPRLLIYRASNLEAGVPDRFSGSGSKTDFTLTISPVEAEDAATYYCQQSREYPYTFGGGTKLEIK

SEQ ID NO: 344 - protein, 5 aa, synthetic construct
  REGION 1..5: note = Description of Artificial Sequence: Synthetic peptide
GYGLS

SEQ ID NO: 345 - protein, 17 aa, synthetic construct
  REGION 1..17: note = Description of Artificial Sequence: Synthetic peptide
MISSGGSYTYYADSVKG

SEQ ID NO: 346 - protein, 10 aa, synthetic construct
  REGION 1..10: note = Description of Artificial Sequence: Synthetic peptide
HGDDPAWFAY

SEQ ID NO: 347 - protein, 12 aa, synthetic construct
  REGION 1..12: note = Description of Artificial Sequence: Synthetic peptide
SVSSSISSNNLH

SEQ ID NO: 348 - protein, 7 aa, synthetic construct
  REGION 1..7: note = Description of Artificial Sequence: Synthetic peptide
GTSNLAS

SEQ ID NO: 349 - protein, 11 aa, synthetic construct
  REGION 1..11: note = Description of Artificial Sequence: Synthetic peptide
QQWSSYPYMYT

SEQ ID NO: 350 - protein, 119 aa, synthetic construct
  REGION 1..119: note = Description of Artificial Sequence: Synthetic polypeptide
EVQLVESGGGVVQPGRSLRLSCSASGFTFSGYGLSWVRQAPGKGLEWVAMISSGGSYTYYADSVKGRFAISRDNAKNTLFLQMDSLRPEDTGVYFCARHGDDPAWFAYWGQGTPVTVSS

SEQ ID NO: 351 - protein, 110 aa, synthetic construct
  REGION 1..110: note = Description of Artificial Sequence: Synthetic polypeptide
DIQLTQSPSSLSASVGDRVTITCSVSSSISSNNLHWYQQKPGKAPKPWIYGTSNLASGVPSRFSGSGSGTDYTFTISSLQPEDIATYYCQQWSSYPYMYTFGQGTKVEIK

SEQ ID NO: 352 - protein, 5 aa, synthetic construct
  REGION 1..5: note = Description of Artificial Sequence: Synthetic peptide
NYWMN

SEQ ID NO: 353 - protein, 19 aa, synthetic construct
  REGION 1..19: note = Description of Artificial Sequence: Synthetic peptide
EIRLKSNNYTTHYAESVKG

SEQ ID NO: 354 - protein, 6 aa, synthetic construct
  REGION 1..6: note = Description of Artificial Sequence: Synthetic peptide
HYYFDY

SEQ ID NO: 355 - protein, 16 aa, synthetic construct
  REGION 1..16: note = Description of Artificial Sequence: Synthetic peptide
RSSKSLLHSNGITYFF

SEQ ID NO: 356 - protein, 7 aa, synthetic construct
  REGION 1..7: note = Description of Artificial Sequence: Synthetic peptide
QMSNLAS

SEQ ID NO: 357 - protein, 9 aa, synthetic construct
  REGION 1..9: note = Description of Artificial Sequence: Synthetic peptide
AQNLELPPT

SEQ ID NO: 358 - protein, 117 aa, synthetic construct
  REGION 1..117: note = Description of Artificial Sequence: Synthetic polypeptide
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSS

SEQ ID NO: 359 - protein, 112 aa, synthetic construct
  REGION 1..112: note = Description of Artificial Sequence: Synthetic polypeptide
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIK

SEQ ID NO: 360 - protein, 5 aa, synthetic construct
  REGION 1..5: note = Description of Artificial Sequence: Synthetic peptide
SYWIG

SEQ ID NO: 361 - protein, 17 aa, synthetic construct
  REGION 1..17: note = Description of Artificial Sequence: Synthetic peptide
IIDPGDSRTRYSPSFQG

SEQ ID NO: 362 - protein, 11 aa, synthetic construct
  REGION 1..11: note = Description of Artificial Sequence: Synthetic peptide
GQLYGGTYMDG

SEQ ID NO: 363 - protein, 14 aa, synthetic construct
  REGION 1..14: note = Description of Artificial Sequence: Synthetic peptide
TGTSSDIGGYNSVS

SEQ ID NO: 364 - protein, 7 aa, synthetic construct
  REGION 1..7: note = Description of Artificial Sequence: Synthetic peptide
GVNNRPS

SEQ ID NO: 365 - protein, 11 aa, synthetic construct
  REGION 1..11: note = Description of Artificial Sequence: Synthetic peptide
SSYDIESATPV

SEQ ID NO: 366 - protein, 120 aa, synthetic construct
  REGION 1..120: note = Description of Artificial Sequence: Synthetic polypeptide
QVELVQSGAEVKKPGESLKISCKGSGYSFTSYWIGWVRQAPGKGLEWMGIIDPGDSRTRYSPSFQGQVTISADKSISTAYLQWSSLKASDTAMYYCARGQLYGGTYMDGWGQGTLVTVSS

SEQ ID NO: 367 - protein, 111 aa, synthetic construct
  REGION 1..111: note = Description of Artificial Sequence: Synthetic polypeptide
DIALTQPASVSGSPGQSITISCTGTSSDIGGYNSVSWYQQHPGKAPKLMIYGVNNRPSGVSNRFSGSKSGNTASLTISGLQAEDEADYYCSSYDIESATPVFGGGTKLTVL

SEQ ID NO: 368 - protein, 5 aa, synthetic construct
  REGION 1..5: note = Description of Artificial Sequence: Synthetic peptide
AYNIH

SEQ ID NO: 369 - protein, 17 aa, synthetic construct
  REGION 1..17: note = Description of Artificial Sequence: Synthetic peptide
SFDPYDGGSSYNQKFKD

SEQ ID NO: 370 - protein, 7 aa, synthetic construct
  REGION 1..7: note = Description of Artificial Sequence: Synthetic peptide
GWYYFDY

SEQ ID NO: 371 - protein, 11 aa, synthetic construct
  REGION 1..11: note = Description of Artificial Sequence: Synthetic peptide
RASKSISKYLA

SEQ ID NO: 372 - protein, 7 aa, synthetic construct
  REGION 1..7: note = Description of Artificial Sequence: Synthetic peptide
SGSTLQS

SEQ ID NO: 373 - protein, 9 aa, synthetic construct
  REGION 1..9: note = Description of Artificial Sequence: Synthetic peptide
QQHDESPYT

SEQ ID NO: 374 - protein, 116 aa, synthetic construct
  REGION 1..116: note = Description of Artificial Sequence: Synthetic polypeptide
QVQLQESGPGLVKPSQTLSLTCTVSGYAFTAYNIHWVRQAPGQGLEWMGSFDPYDGGSSYNQKFKDRLTISKDTSKNQVVLTMTNMDPVDTATYYCARGWYYFDYWGHGTLVTVSS

SEQ ID NO: 375 - protein, 107 aa, synthetic construct
  REGION 1..107: note = Description of Artificial Sequence: Synthetic polypeptide
DIVMTQTPLSLPVTPGEPASISCRASKSISKYLAWYQQKPGQAPRLLIYSGSTLQSGIPPRFSGSGYGTDFTLTINNIESEDAAYYFCQQHDESPYTFGEGTKVEIK

SEQ ID NO: 376 - protein, 11 aa, synthetic construct
  REGION 1..11: note = Description of Artificial Sequence: Synthetic peptide
GSIKSGSYYWG

SEQ ID NO: 377 - protein, 16 aa, synthetic construct
  REGION 1..16: note = Description of Artificial Sequence: Synthetic peptide
NIYYSGSTYYNPSLRS

SEQ ID NO: 378 - protein, 12 aa, synthetic construct
  REGION 1..12: note = Description of Artificial Sequence: Synthetic peptide
AREGSYPNQFDP

SEQ ID NO: 379 - protein, 11 aa, synthetic construct
  REGION 1..11: note = Description of Artificial Sequence: Synthetic peptide
RASQSVSSNLA

SEQ ID NO: 380 - protein, 7 aa, synthetic construct
  REGION 1..7: note = Description of Artificial Sequence: Synthetic peptide
GASTRAT

SEQ ID NO: 381 - protein, 9 aa, synthetic construct
  REGION 1..9: note = Description of Artificial Sequence: Synthetic peptide
QQYHSFPFT

SEQ ID NO: 382 - protein, 120 aa, synthetic construct
  REGION 1..120: note = Description of Artificial Sequence: Synthetic polypeptide
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSS

SEQ ID NO: 383 - protein, 107 aa, synthetic construct
  REGION 1..107: note = Description of Artificial Sequence: Synthetic polypeptide
EIVMTQSPATLSVSPGERATLSCRASQSVSSNLAWYQQKPGQAPRLLIYGASTRATGIPARFSGSGSGTEFTLTISSLQSEDFAVYYCQQYHSFPFTFGGGTKVEIK

SEQ ID NO: 384 - protein, 5 aa, synthetic construct
  REGION 1..5: note = Description of Artificial Sequence: Synthetic peptide
SFGMH

SEQ ID NO: 385 - protein, 11 aa, synthetic construct
  REGION 1..11: note = Description of Artificial Sequence: Synthetic peptide
YISSDSSAIYY

SEQ ID NO: 386 - protein, 12 aa, synthetic construct
  REGION 1..12: note = Description of Artificial Sequence: Synthetic peptide
GRENIYYGSRLD

SEQ ID NO: 387 - protein, 7 aa, synthetic construct
  REGION 1..7: note = Description of Artificial Sequence: Synthetic peptide
KASQNVD

SEQ ID NO: 388 - protein, 11 aa, synthetic construct
  REGION 1..11: note = Description of Artificial Sequence: Synthetic peptide
SASYRYSGVPD

SEQ ID NO: 389 - protein, 12 aa, synthetic construct
  REGION 1..12: note = Description of Artificial Sequence: Synthetic peptide
QQYNNYPFTFGS

SEQ ID NO: 390 - protein, 122 aa, synthetic construct
  REGION 1..122: note = Description of Artificial Sequence: Synthetic polypeptide
DVQLVESGGGLVQPGGSRKLSCAASGFTFSSFGMHWVRQAPEKGLEWVAYISSDSSAIYYADTVKGRFTISRDNPKNTLFLQMTSLRSEDTAMYYCGRGRENIYYGSRLDYWGQGTTLTVSS

SEQ ID NO: 391 - protein, 107 aa, synthetic construct
  REGION 1..107: note = Description of Artificial Sequence: Synthetic polypeptide
DIAMTQSQKFMSTSVGDRVSVTCKASQNVDTNVAWYQQKPGQSPKALIYSASYRYSGVPDRFTGSGSGTDFTLTINNVQSEDLAEYFCQQYNNYPFTFGSGTKLEIK

SEQ ID NO: 392 - protein, 10 aa, synthetic construct
  REGION 1..10: note = Description of Artificial Sequence: Synthetic peptide
SYWMQWVRQA

SEQ ID NO: 393 - protein, 11 aa, synthetic construct
  REGION 1..11: note = Description of Artificial Sequence: Synthetic peptide
TIYPGDGDTRY

SEQ ID NO: 394 - protein, 12 aa, synthetic construct
  REGION 1..12: note = Description of Artificial Sequence: Synthetic peptide
RGIPRLWYFDVM

SEQ ID NO: 395 - protein, 10 aa, synthetic construct
  REGION 1..10: note = Description of Artificial Sequence: Synthetic peptide
ITCRASQDIS

SEQ ID NO: 396 - protein, 11 aa, synthetic construct
  REGION 1..11: note = Description of Artificial Sequence: Synthetic peptide
YTSRLHSGVPS

SEQ ID NO: 397 - protein, 12 aa, synthetic construct
  REGION 1..12: note = Description of Artificial Sequence: Synthetic peptide
QQGNTLPPFTGG

SEQ ID NO: 398 - protein, 122 aa, synthetic construct; identical to SEQ ID NO: 390
  REGION 1..122: note = Description of Artificial Sequence: Synthetic polypeptide

SEQ ID NO: 399 - protein, 107 aa, synthetic construct; identical to SEQ ID NO: 391
  REGION 1..107: note = Description of Artificial Sequence: Synthetic polypeptide

SEQ ID NO: 400 - protein, 10 aa, synthetic construct
  REGION 1..10: note = Description of Artificial Sequence: Synthetic peptide
SYGMSWVRQA

SEQ ID NO: 401 - protein, 10 aa, synthetic construct
  REGION 1..10: note = Description of Artificial Sequence: Synthetic peptide
INSGGSNTYY

SEQ ID NO: 402 - protein, 9 aa, synthetic construct
  REGION 1..9: note = Description of Artificial Sequence: Synthetic peptide
HDGGAMDYW

SEQ ID NO: 403 - protein, 14 aa, synthetic construct
  REGION 1..14: note = Description of Artificial Sequence: Synthetic peptide
ITCRASESIYSYLA

SEQ ID NO: 404 - protein, 7 aa, synthetic construct
  REGION 1..7: note = Description of Artificial Sequence: Synthetic peptide
NTKTLPE

SEQ ID NO: 405 - protein, 11 aa, synthetic construct
  REGION 1..11: note = Description of Artificial Sequence: Synthetic peptide
HHYGTPPWTFG

SEQ ID NO: 406 - protein, 117 aa, synthetic construct
  REGION 1..117: note = Description of Artificial Sequence: Synthetic polypeptide
EVQLVESGGGLVKPGGSLRLSCAASGFTFSSYGMSWVRQAPGKGLEWVATINSGGSNTYYPDSLKGRFTISRDNAKNSLYLQMNSLRAEDTAVYYCARHDGGAMDYWGQGTTVTVSS

SEQ ID NO: 407 - protein, 108 aa, synthetic construct
  REGION 1..108: note = Description of Artificial Sequence: Synthetic polypeptide
DIQMTQSPSSLSASVGDRVTITCRASESIYSYLAWYQQKPGKAPKLLVYNTKTLPEGVPSRFSGSGSGTDFTLTISSLQPEDFATYYCQHHYGTPPWTFGQGTRLEIK

SEQ ID NO: 408 - protein, 10 aa, synthetic construct
  REGION 1..10: note = Description of Artificial Sequence: Synthetic peptide
SFGMHWVRQA

SEQ ID NO: 409 - protein, 20 aa, synthetic construct
  REGION 1..20: note = Description of Artificial Sequence: Synthetic peptide
ISSGSGTIYYADTVKGRFTI

SEQ ID NO: 410 - protein, 12 aa, synthetic construct
  REGION 1..12: note = Description of Artificial Sequence: Synthetic peptide
HGYRYEGFDYWG

SEQ ID NO: 411 - protein, 14 aa, synthetic construct
  REGION 1..14: note = Description of Artificial Sequence: Synthetic peptide
ITCKASQNVDTNVA

SEQ ID NO: 412 - protein, 11 aa, synthetic construct
  REGION 1..11: note = Description of Artificial Sequence: Synthetic peptide
SASYRYSGVPS

SEQ ID NO: 413 - protein, 12 aa, synthetic construct
  REGION 1..12: note = Description of Artificial Sequence: Synthetic peptide
QQYNNYPFTFGQ

SEQ ID NO: 414 - protein, 119 aa, synthetic construct
  REGION 1..119: note = Description of Artificial Sequence: Synthetic polypeptide
EVQLVESGGGLVQPGGSLRLSCAASGFTFSSFGMHWVRQAPGKGLEWVAYISSGSGTIYYADTVKGRFTISRDNAKNSLYLQMNSLRAEDTAVYYCARHGYRYEGFDYWGQGTTVTVSS

SEQ ID NO: 415 - protein, 107 aa, synthetic construct
  REGION 1..107: note = Description of Artificial Sequence: Synthetic polypeptide
DIQMTQSPSFLSASVGDRVTITCKASQNVDTNVAWYQQKPGKAPKALIYSASYRYSGVPSRFSGSGSGTDFTLTISSLQPEDFAEYFCQQYNNYPFTFGQGTKLEIK

SEQ ID NO: 416 - protein, 5 aa, synthetic construct
  REGION 1..5: note = Description of Artificial Sequence: Synthetic peptide
NYVMH

SEQ ID NO: 417 - protein, 17 aa, synthetic construct
  REGION 1..17: note = Description of Artificial Sequence: Synthetic peptide
YINPYNDDVKYNEKFKG

SEQ ID NO: 418 - protein, 13 aa, synthetic construct
  REGION 1..13: note = Description of Artificial Sequence: Synthetic peptide
WGYYGSPLYYFDY

SEQ ID NO: 419 - protein, 10 aa, synthetic construct
  REGION 1..10: note = Description of Artificial Sequence: Synthetic peptide
RASSRLIYMH

SEQ ID NO: 420 - protein, 7 aa, synthetic construct
  REGION 1..7: note = Description of Artificial Sequence: Synthetic peptide
ATSNLAS

SEQ ID NO: 421 - protein, 9 aa, synthetic construct
  REGION 1..9: note = Description of Artificial Sequence: Synthetic peptide
QQWNSNPPT

SEQ ID NO: 422 - protein, 122 aa, synthetic construct
  REGION 1..122: note = Description of Artificial Sequence: Synthetic polypeptide
EVQLQQSGPELVKPGASVKMSCKASGYTFTNYVMHWVKQKPGQGLEWIGYINPYNDDVKYNEKFKGKATQTSDKSSSTAYMELSSLTSEDSAVYYCARWGYYGSPLYYFDYWGQGTTLTVSS

SEQ ID NO: 423 - protein, 106 aa, synthetic construct
  REGION 1..106: note = Description of Artificial Sequence: Synthetic polypeptide
QIVLSQSPTILSASPGEKVTMTCRASSRLIYMHWYQQKPGSSPKPWIYATSNLASGVPARFSGSGSGTSYSLTISRVEAEDAATYYCQQWNSNPPTFGTGTKLELK

SEQ ID NO: 424 - protein, 5 aa, synthetic construct
  REGION 1..5: note = Description of Artificial Sequence: Synthetic peptide
NYVMH

SEQ ID NO: 425 - protein, 17 aa, synthetic construct
  REGION 1..17: note = Description of Artificial Sequence: Synthetic peptide
YINPYNDDVKYNEKFKG

SEQ ID NO: 426 - protein, 13 aa, synthetic construct
  REGION 1..13: note = Description of Artificial Sequence: Synthetic peptide
WGYYGSPLYYFDY

SEQ ID NO: 427 - protein, 10 aa, synthetic construct
  REGION 1..10: note = Description of Artificial Sequence: Synthetic peptide
RASSRLIYMH

SEQ ID NO: 428 - protein, 7 aa, synthetic construct
  REGION 1..7: note = Description of Artificial Sequence: Synthetic peptide
ATSNLAS

SEQ ID NO: 429 - protein, 9 aa, synthetic construct
  REGION 1..9: note = Description of Artificial Sequence: Synthetic peptide
QQWNSNPPT

SEQ ID NO: 430 - protein, 122 aa, synthetic construct
  REGION 1..122: note = Description of Artificial Sequence: Synthetic polypeptide
QVQLVQSGAEVKKPGSSVKVSCKASGYTFTNYVMHWVRQAPGQGLEWMGYINPYNDDVKYNEKFKGRVTITADESTSTAYMELSSLRSEDTAVYYCARWGYYGSPLYYFDYWGQGTLVTVSS

SEQ ID NO: 431 - protein, 106 aa, synthetic construct
  REGION 1..106: note = Description of Artificial Sequence: Synthetic polypeptide
EIVLTQSPATLSLSPGERATLSCRASSRLIYMHWYQQKPGQAPRPLIYATSNLASGIPARFSGSGSGTDFTLTISSLEPEDFAVYYCQQWNSNPPTFGQGTKVEIK

SEQ ID NO: 432 - protein, 10 aa, synthetic construct
  REGION 1..10: note = Description of Artificial Sequence: Synthetic peptide
GYSFTSYTIH

SEQ ID NO: 433 - protein, 17 aa, synthetic construct
  REGION 1..17: note = Description of Artificial Sequence: Synthetic peptide
YINPNSRNTDYAQKFQG

SEQ ID NO: 434 - protein, 12 aa, synthetic construct
  REGION 1..12: note = Description of Artificial Sequence: Synthetic peptide
YSGSTPYWYFDV

SEQ ID NO: 435 - protein, 10 aa, synthetic construct
  REGION 1..10: note = Description of Artificial Sequence: Synthetic peptide
RASSSVSYMN

SEQ ID NO: 436 - protein, 7 aa, synthetic construct
  REGION 1..7: note = Description of Artificial Sequence: Synthetic peptide
ATSNLAS

SEQ ID NO: 437 - protein, 9 aa, synthetic construct
  REGION 1..9: note = Description of Artificial Sequence: Synthetic peptide
QQWSSNPLT

SEQ ID NO: 438 - protein, 121 aa, synthetic construct
  REGION 1..121: note = Description of Artificial Sequence: Synthetic polypeptide
EVQLVQSGAEVKKPGSSVKVSCKASGYSFTSYTIHWVRQAPGQGLEWMGYINPNSRNTDYAQKFQGRVTLTADKSTSTAYMELSSLRSEDTAVYYCARYSGSTPYWYFDVWGQGTTVTVSS

SEQ ID NO: 439 - protein, 107 aa, synthetic construct; identical to SEQ ID NO: 231
  REGION 1..107: note = Description of Artificial Sequence: Synthetic polypeptide

SEQ ID NO: 440 - protein, 10 aa, synthetic construct
  REGION 1..10: note = Description of Artificial Sequence: Synthetic peptide
GYTFSSYWMH

SEQ ID NO: 441 - protein, 17 aa, synthetic construct
  REGION 1..17: note = Description of Artificial Sequence: Synthetic peptide
LIHPDSGSTNYNEMFKN

SEQ ID NO: 442 - protein, 7 aa, synthetic construct
  REGION 1..7: note = Description of Artificial Sequence: Synthetic peptide
GGRLYFD

SEQ ID NO: 443 - protein, 16 aa, synthetic construct
  REGION 1..16: note = Description of Artificial Sequence: Synthetic peptide
RSSQSLVHSNGDTYLR

SEQ ID NO: 444 - protein, 7 aa, synthetic construct
  REGION 1..7: note = Description of Artificial Sequence: Synthetic peptide
KVSNRFS

SEQ ID NO: 445 - protein, 9 aa, synthetic construct
  REGION 1..9: note = Description of Artificial Sequence: Synthetic peptide
SQSTHVPYT

SEQ ID NO: 446 - protein, 117 aa, synthetic construct
  REGION 1..117: note = Description of Artificial Sequence: Synthetic polypeptide
EVQLVQSGAEVKKPGSSVKVSCKASGYTFSSYWMHWVRQAPGQGLEWIGLIHPDSGSTNYNEMFKNRATLTVDRSTSTAYVELSSLRSEDTAVYFCAGGGRLYFDYWGQGTTVTVSS

SEQ ID NO: 447 - protein, 112 aa, synthetic construct
  REGION 1..112: note = Description of Artificial Sequence: Synthetic polypeptide
DVVMTQSPLSLPVTPGEPASISCRSSQSLVHSNGDTYLRWYLQKPGQSPQLLIYKVSNRFSGVPDRFSGSGSGTDFTLKISRVEAEDVGVYYCSQSTHVPYTFGGGTKVEIK

SEQ ID NO: 448 - protein, 10 aa, synthetic construct
  REGION 1..10: note = Description of Artificial Sequence: Synthetic peptide
GYTFSSYWMH

SEQ ID NO: 449 - protein, 17 aa, synthetic construct
  REGION 1..17: note = Description of Artificial Sequence: Synthetic peptide
LIHPESGSTNYNEMFKN

SEQ ID NO: 450 - protein, 8 aa, synthetic construct
  REGION 1..8: note = Description of Artificial Sequence: Synthetic peptide
GGRLYFDY

SEQ ID NO: 451 - protein, 16 aa, synthetic construct
  REGION 1..16: note = Description of Artificial Sequence: Synthetic peptide
RSSQSLVHSNQDTYLR

SEQ ID NO: 452 - protein, 7 aa, synthetic construct
  REGION 1..7: note = Description of Artificial Sequence: Synthetic peptide
KVSNRFS

SEQ ID NO: 453 - protein, 9 aa, synthetic construct
  REGION 1..9: note = Description of Artificial Sequence: Synthetic peptide
SQSTHVPYT

SEQ ID NO: 454 - protein, 117 aa, synthetic construct
  REGION 1..117: note = Description of Artificial Sequence: Synthetic polypeptide
EVQLVQSGAEVKKPGSSVKVSCKASGYTFSSYWMHWVRQAPGQGLEWIGLIHPESGSTNYNEMFKNRATLTVDRSTSTAYMELSSLRSEDTAVYYCAGGGRLYFDYWGQGTTVTVSS

SEQ ID NO: 455 - protein, 113 aa, synthetic construct
  REGION 1..113: note = Description of Artificial Sequence: Synthetic polypeptide
DIVMTQSPLSLPVTPGEPASISCRSSQSLVHSNQDTYLRWYLQKPGQSPQLLIYKVSNRFSGVPDRFSGSGSGTDFTLKKISRVEAEDVGVYYCSQSTHVPYTFGGGTKVEIK

SEQ ID NO: 456 - protein, 12 aa, synthetic construct
  REGION 1..12: note = Description of Artificial Sequence: Synthetic peptide
TGYSITSGYSWH

SEQ ID NO: 457 - protein, 16 aa, synthetic construct
  REGION 1..16: note = Description of Artificial Sequence: Synthetic peptide
YIHSSGSTNYNPSLKS

SEQ ID NO: 458 - protein, 7 aa, synthetic construct
  REGION 1..7: note = Description of Artificial Sequence: Synthetic peptide
YDDYFEY

SEQ ID NO: 459 - protein, 12 aa, synthetic construct
  REGION 1..12: note = Description of Artificial Sequence: Synthetic peptide
KASQNVGFNVAW

SEQ ID NO: 460 - protein, 7 aa, synthetic construct
  REGION 1..7: note = Description of Artificial Sequence: Synthetic peptide
SASYRYS

SEQ ID NO: 461 - protein, 9 aa, synthetic construct
  REGION 1..9: note = Description of Artificial Sequence: Synthetic peptide
QQYNWYPFT

SEQ ID NO: 462 - protein, 116 aa, synthetic construct; identical to SEQ ID NO: 230
  REGION 1..116: note = Description of Artificial Sequence: Synthetic polypeptide

SEQ ID NO: 463 - protein, 108 aa, synthetic construct
  REGION 1..108: note = Description of Artificial Sequence: Synthetic polypeptide
DIQMTQSPSSLSASVGDRVTITCKASQNVGGFNVAWYQQKPGKSPKALIYSASYRYSGVPSRFSGSGSGTDFTLTISSLQPEDFAEYFCQQYNWYPFTFGQGTKLEIK

SEQ ID NO: 464 - protein, 5 aa, synthetic construct
  REGION 1..5: note = Description of Artificial Sequence: Synthetic peptide
NYDIN

SEQ ID NO: 465 - protein, 20 aa, synthetic construct
  REGION 1..20: note = Description of Artificial Sequence: Synthetic peptide
WIGWIFPGDDSTQYNEKFKG

SEQ ID NO: 466 - protein, 9 aa, synthetic construct
  REGION 1..9: note = Description of Artificial Sequence: Synthetic peptide
QTTGTWFAY

SEQ ID NO: 467 - protein, 11 aa, synthetic construct
  REGION 1..11: note = Description of Artificial Sequence: Synthetic peptide
RASQSISDYLY

SEQ ID NO: 468 - protein, 7 aa, synthetic construct
  REGION 1..7: note = Description of Artificial Sequence: Synthetic peptide
YASQSIS

SEQ ID NO: 469 - protein, 9 aa, synthetic construct
  REGION 1..9: note = Description of Artificial Sequence: Synthetic peptide
CQNGHSFPL

SEQ ID NO: 470 - protein, 118 aa, synthetic construct
  REGION 1..118: note = Description of Artificial Sequence: Synthetic polypeptide
QVQLVQSGAEVVKPGASVKLSCKTSGYTFTNYDINWVRQRPGQGLEWIGWIFPGDDSTQYNEKFKGKATLTTDTSTSTAYMELSSLRSEDTAVYFCARQTTGTWFAYWGQGTLVTVSS

SEQ ID NO: 471 - protein, 107 aa, synthetic construct
  REGION 1..107: note = Description of Artificial Sequence: Synthetic polypeptide
EIVMTQSPATLSVSPGERVTLSCRASQSISDYLYWYQQKSHESPRLLIKYASQSISGIPARFSGSGSGSEFTLTINSVEPEDVGVYYCQNGHSFPLTFGQGTKLELK

SEQ ID NO: 472 - protein, 119 aa, synthetic construct
  REGION 1..119: note = Description of Artificial Sequence: Synthetic polypeptide
QVQLQQSGAEVKKPGSSVKVSCKASGGTFSSYAISWVRQAPGQGLEWMGGIIPILGIANYAQKFQGRVTITADESTSTAYMELSSLRSEDTAVYYCARGGSGSYHMDVWGKGTTVTVSS

SEQ ID NO: 473 - protein, 109 aa, synthetic construct
  REGION 1..109: note = Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPPRITFGQGTRLEIK

SEQ ID NO: 474 - protein, 5 aa, synthetic construct
  REGION 1..5: note = Description of Artificial Sequence: Synthetic peptide
IYNVH

SEQ ID NO: 475 - protein, 17 aa, synthetic construct
  REGION 1..17: note = Description of Artificial Sequence: Synthetic peptide
TIFPGNGDTSYNQKFKD

SEQ ID NO: 476 - protein, 10 aa, synthetic construct
  REGION 1..10: note = Description of Artificial Sequence: Synthetic peptide
WDDGNVGFAH

SEQ ID NO: 477 - protein, 11 aa, synthetic construct
  REGION 1..11: note = Description of Artificial Sequence: Synthetic peptide
RASENINNYLT

SEQ ID NO: 478 - protein, 7 aa, synthetic construct
  REGION 1..7: note = Description of Artificial Sequence: Synthetic peptide
HAKTLAE

SEQ ID NO: 479 - protein, 9 aa, synthetic construct
  REGION 1..9: note = Description of Artificial Sequence: Synthetic peptide
QHHYGTPPT

SEQ ID NO: 480 - protein, 119 aa, synthetic construct
  REGION 1..119: note = Description of Artificial Sequence: Synthetic polypeptide
QVQLQQPGAELVKPGASVKMSCKASGYTFTIYNVHWIKQTPGQGLEWMGTIFPGNGDTSYNQKFKDKATLTTDKSSKTAYMQLNSLTSEDSAVYYCARWDDGNVGFAHWGQGTLVTVSA

SEQ ID NO: 481 - protein, 107 aa, synthetic construct
  REGION 1..107: note = Description of Artificial Sequence: Synthetic polypeptide
DIQMTQSPASLSASVGETVTITCRASENINNYLTWFQQKQGKSPQLLVYHAKTLAEGVPSRFSGSGSGTQFSLKINSLQPEDFGSYYCQHHYGTPPTFGGGTKLEIK

SEQ ID NO: 482 - protein, 119 aa, synthetic construct
  REGION 1..119: note = Description of Artificial Sequence: Synthetic polypeptide
EVQLVQSGAEVKKPGASVKVSCKASGYTFTIYNVHWVRQAPGQGLEWMGTIFPGNGDTSYNQKFKDKVTMTTDTSTSTAYMELSSLRSEDTAVYYCARWDDGNVGFAHWGQGTLVTVSS

SEQ ID NO: 483 - protein, 107 aa, synthetic construct
  REGION 1..107: note = Description of Artificial Sequence: Synthetic polypeptide
DIQMTQSPSSLSASVGDRVTITCRASENINNYLTWFQQKQGKSPQLLIYHAKTLAEGVPSRFSGSGSGTDFTLTISSLQPEDFATYYCQHHYGTPPTFGGGTKVEIK

SEQ ID NO: 484 - protein, 119 aa, synthetic construct
  REGION 1..119: note = Description of Artificial Sequence: Synthetic polypeptide
EVQLVQSGAEVKKPGASVKVSCKASGYTFTIYNVHWIRQAPGQGLEWMGTIFPGNGDTSYNQKFKDRATLTTDKSTKTAYMELRSLRSDDTAVYYCARWDDGNVGFAHWGQGTLVTVSS

SEQ ID NO: 485 - protein, 107 aa, synthetic construct
  REGION 1..107: note = Description of Artificial Sequence: Synthetic polypeptide
DIQMTQSPSSLSASVGDRVTITCRASENINNYLTWFQQKPGKAPKLLVYHAKTLAEGVPSRFSGSGSGTQFTLTISSLQPEDFATYYCQHHYGTPPTFGQGTKLEIK

SEQ ID NO: 486 - protein, 447 aa, synthetic construct
  REGION 1..447: note = Description of Artificial Sequence: Synthetic polypeptide
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 487 - protein, 220 aa, synthetic construct
  REGION 1..220: note = Description of Artificial Sequence: Synthetic polypeptide
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 488 - protein, 445 aa, synthetic construct
  REGION 1..445: note = Description of Artificial Sequence: Synthetic polypeptide
EVQLLESGGGLVQPGGSLRLSCAASGFTFSHYVMAWVRQAPGKGLEWVSSISSSGGWTLYADSVKGRFTISRDNSKNTLYLQMNSLRAEDTAVYYCTRGLKMATIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 489 - protein, 217 aa, synthetic construct
  REGION 1..217: note = Description of Artificial Sequence: Synthetic polypeptide
QSALTQPASVSGSPGQSITISCTGTSSDVGSYNVVSWYQQHPGKAPKLIIYEVSQRPSGVSNRFSGSKSGNTASLTISGLQTEDEADYYCCSYAGSSIFVIFGGGTKVTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 490 - protein, 447 aa, synthetic construct
  REGION 1..447: note = Description of Artificial Sequence: Synthetic polypeptide
EVQLLESGGGLVQPGGSLRLSCAASGFTFSSYAMSWVRQAPGKGLEWVSAINSQGKSTYYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 491 - protein, 214 aa, synthetic construct
  REGION 1..214: note = Description of Artificial Sequence: Synthetic polypeptide
DIQMTQSPSSLSASVGDRVTITCRASQGISNWLAWYQQKPGKAPKLLIYGASSLQSGVPSRFSGSGSGTDFTLTISSLQPEDFATYYCQQYSSFPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 492 - protein, 449 aa, synthetic construct
  REGION 1..449: note = Description of Artificial Sequence: Synthetic polypeptide
QVQLVQSGAEVKKPGASVKVSCKASGYTFRSSYISWVRQAPGQGLEWMGWIYAGTGSPSYNQKLQGRVTMTTDTSTSTAYMELRSLRSDDTAVYYCARHRDYYSNSLTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 493 - protein, 220 aa, synthetic construct
  REGION 1..220: note = Description of Artificial Sequence: Synthetic polypeptide
DIVMTQSPDSLAVSLGERATINCKSSQSVLNSGNQKNYLTWYQQKPGQPPKLLIYWASTRESGVPDRFSGSGSGTDFTLTISSLQAEDVAVYYCQSDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 494 - protein, 7 aa, synthetic construct
  REGION 1..7: note = Description of Artificial Sequence: Synthetic peptide
TSNMGVG

SEQ ID NO: 495 - protein, 16 aa, synthetic construct
  REGION 1..16: note = Description of Artificial Sequence: Synthetic peptide
HIWWDDDKYYSPSLKS

SEQ ID NO: 496 - protein, 10 aa, synthetic construct
  REGION 1..10: note = Description of Artificial Sequence: Synthetic peptide
SNYGYAWFAY

SEQ ID NO: 497 - protein, 11 aa, synthetic construct
  REGION 1..11: note = Description of Artificial Sequence: Synthetic peptide
KASQDIYPYLN

SEQ ID NO: 498 - protein, 7 aa, synthetic construct
  REGION 1..7: note = Description of Artificial Sequence: Synthetic peptide
RTNRLLD

SEQ ID NO: 499 - protein, 9 aa, synthetic construct
  REGION 1..9: note = Description of Artificial Sequence: Synthetic peptide
LQYDEFPLT

SEQ ID NO: 500 - protein, 120 aa, synthetic construct
  REGION 1..120: note = Description of Artificial Sequence: Synthetic polypeptide
QITLKESGPTLVKPTQTLTLTCTFSGFSLSTSNMGVGWIRQPPGKALEWLAHIWWDDDKYYSPSLKSRLTITKDTSKNQVVLTMTNMDPVDTATYYCVRSNYGYAWFAYWGQGTLVTVSS

SEQ ID NO: 501 - protein, 107 aa, synthetic construct
  REGION 1..107: note = Description of Artificial Sequence: Synthetic polypeptide
DIQMTQSPSSLSASVGDRVTITCKASQDIYPYLNWFQQKPGKAPKTLIYRTNRLLDGVPSRFSGSGSGTDFTFTISSLQPEDIATYYCLQYDEFPLTFGAGTKLEIK

SEQ ID NO: 502 - protein, 5 aa, synthetic construct
  REGION 1..5: note = Description of Artificial Sequence: Synthetic peptide
DYAVH

SEQ ID NO: 503 - protein, 17 aa, synthetic construct
  REGION 1..17: note = Description of Artificial Sequence: Synthetic peptide
VISTYNDYTYNNQDFKG

SEQ ID NO: 504 - protein, 10 aa, synthetic construct
  REGION 1..10: note = Description of Artificial Sequence: Synthetic peptide
GNSYFYALDY

SEQ ID NO: 505 - protein, 15 aa, synthetic construct
  REGION 1..15: note = Description of Artificial Sequence: Synthetic peptide
RASESVDSYGKSFMH

SEQ ID NO: 506 - protein, 7 aa, synthetic construct
  REGION 1..7: note = Description of Artificial Sequence: Synthetic peptide
RASNLES

SEQ ID NO: 507 - protein, 9 aa, synthetic construct
  REGION 1..9: note = Description of Artificial Sequence: Synthetic peptide
QQSNEDPWT

SEQ ID NO: 508 - protein, 119 aa, synthetic construct
  REGION 1..119: note = Description of Artificial Sequence: Synthetic polypeptide
QVQLVQSGPEVKKPGASVKVSCKASGYTFTDYAVHWVRQAPGKRLEWIGVISTYNDYTYNNQDFKGRVTMTRDTSASTAYMELSRLRSEDTAVYYCARGNSYFYALDYWGQGTSVTVSS

SEQ ID NO: 509 - protein, 111 aa, synthetic construct
  REGION 1..111: note = Description of Artificial Sequence: Synthetic polypeptide
EIVLTQSPATLSLSPGERATLSCRASESVDSYGKSFMHWYQQKPGQAPRLLIYRASNLESGIPARFSGSGSGTDFTLTISSLEPEDFAVYYCQQSNEDPWTFGGGTKLEIK

SEQ ID NO: 510 - protein, 5 aa, synthetic construct
  REGION 1..5: note = Description of Artificial Sequence: Synthetic peptide
RYWMS

SEQ ID NO: 511 - protein, 17 aa, synthetic construct
  REGION 1..17: note = Description of Artificial Sequence: Synthetic peptide
DLNPDSSAINYVDSVKG

SEQ ID NO: 512 - protein, 11 aa, synthetic construct
  REGION 1..11: note = Description of Artificial Sequence: Synthetic peptide
ITTLVPYTMDF

SEQ ID NO: 513 - protein, 11 aa, synthetic construct
  REGION 1..11: note = Description of Artificial Sequence: Synthetic peptide
ITNTDIDDDMN

SEQ ID NO: 514 - protein, 7 aa, synthetic construct
  REGION 1..7: note = Description of Artificial Sequence: Synthetic peptide
EGNGLRP

SEQ ID NO: 515 - protein, 9 aa, synthetic construct
  REGION 1..9: note = Description of Artificial Sequence: Synthetic peptide
LQSDNLPLT

SEQ ID NO: 516 - protein, 120 aa, synthetic construct
  REGION 1..120: note = Description of Artificial Sequence: Synthetic polypeptide
EVQLVESGGGLVQPGGSLRLSCAASGFDFSRYWMSWVRQAPGKGLEWIGDLNPDSSAINYVDSVKGRFTISRDNAKNSLYLQMNSLRAEDTAVYYCTLITTLVPYTMDFWGQGTSVTVSS

SEQ ID NO: 517 - protein, 107 aa, synthetic construct
  REGION 1..107: note = Description of Artificial Sequence: Synthetic polypeptide
ETTLTQSPAFMSATPGDKVNISCITNTDIDDDMNWYQQKPGEAAILLISEGNGLRPGIPPRFSGSGYGTDFTLTINNIESEDAAYYFCLQSDNLPLTFGSGTKLEIK

SEQ ID NO: 518 - protein, 5 aa, synthetic construct
  REGION 1..5: note = Description of Artificial Sequence: Synthetic peptide
DYYMH

SEQ ID NO: 519 - protein, 17 aa, synthetic construct
  REGION 1..17: note = Description of Artificial Sequence: Synthetic peptide
WIDPENGDTEYGPKFQG

SEQ ID NO: 520 - protein, 11 aa, synthetic construct
  REGION 1..11: note = Description of Artificial Sequence: Synthetic peptide
HNAHYGTWFAY

SEQ ID NO: 521 - protein, 16 aa, synthetic construct
  REGION 1..16: note = Description of Artificial Sequence: Synthetic peptide
RSSQSLLHSSGNTYLE

SEQ ID NO: 522 - protein, 7 aa, synthetic construct
  REGION 1..7: note = Description of Artificial Sequence: Synthetic peptide
KISTRFS

SEQ ID NO: 523 - protein, 9 aa, synthetic construct
  REGION 1..9: note = Description of Artificial Sequence: Synthetic peptide
FQGSHVPYT

SEQ ID NO: 524 - protein, 120 aa, synthetic construct
  REGION 1..120: note = Description of Artificial Sequence: Synthetic polypeptide
QVQLVQSGAEVKKPGASVKVSCKASGLTIEDYYMHWVRQAPGQGLEWMGWIDPENGDTEYGPKFQGRVTMTRDTSINTAYMELSRLRSDDTAVYYCAVHNAHYGTWFAYWGQGTLVTVSS

SEQ ID NO: 525 - protein, 112 aa, synthetic construct
  REGION 1..112: note = Description of Artificial Sequence: Synthetic polypeptide
DVVMTQSPLSLPVTLGQPASISCRSSQSLLHSSGNTYLEWYQQRPGQSPRPLIYKISTRFSGVPDRFSGSGSGTDFTLKISRVEAEDVGVYYCFQGSHVPYTFGGGTKVEIK

SEQ ID NO: 526 - protein, 5 aa, synthetic construct
  REGION 1..5: note = Description of Artificial Sequence: Synthetic peptide
DYNVN

SEQ ID NO: 527 - protein, 17 aa, synthetic construct
  REGION 1..17: note = Description of Artificial Sequence: Synthetic peptide
VINPKYGTTRYNQKFKG

SEQ ID NO: 528 - protein, 7 aa, synthetic construct
  REGION 1..7: note = Description of Artificial Sequence: Synthetic peptide
GLNAWDY

SEQ ID NO: 529 - protein, 11 aa, synthetic construct
  REGION 1..11: note = Description of Artificial Sequence: Synthetic peptide
GASENIYGALN

SEQ ID NO: 530 - protein, 7 aa, synthetic construct
  REGION 1..7: note = Description of Artificial Sequence: Synthetic peptide
GATNLED

SEQ ID NO: 531 - protein, 9 aa, synthetic construct
  REGION 1..9: note = Description of Artificial Sequence: Synthetic peptide
QNVLTTPYT

SEQ ID NO: 532 - protein, 116 aa, synthetic construct
  REGION 1..116: note = Description of Artificial Sequence: Synthetic polypeptide
QFQLVQSGAEVKKPGASVKVSCKASGYSFTDYNVNWVRQAPGQGLEWIGVINPKYGTTRYNQKFKGRATLTVDKSTSTAYMELSSLRSEDTAVYYCTRGLNAWDYWGQGTLVTVSS

SEQ ID NO: 533 - protein, 107 aa, synthetic construct
  REGION 1..107: note = Description of Artificial Sequence: Synthetic polypeptide
DIQMTQSPSSLSASVGDRVTITCGASENIYGALNWYQQKPGKAPKLLIYGATNLEDGVPSRFSGSGSGRDYTFTISSLQPEDIATYYCQNVLTTPYTFGQGTKLEIK

SEQ ID NO: 534 - protein, 5 aa, synthetic construct
  REGION 1..5: note = Description of Artificial Sequence: Synthetic peptide
GYFMN

SEQ ID NO: 535 - protein, 17 aa, synthetic construct
  REGION 1..17: note = Description of Artificial Sequence: Synthetic peptide
LINPYNGDSFYNQKFKG

SEQ ID NO: 536 - protein, 8 aa, synthetic construct
  REGION 1..8: note = Description of Artificial Sequence: Synthetic peptide
GLRRDFDY

SEQ ID NO: 537 - protein, 16 aa, synthetic construct
  REGION 1..16: note = Description of Artificial Sequence: Synthetic peptide
KSSQSLLDSDGKTYLN

SEQ ID NO: 538 - protein, 7 aa, synthetic construct
  REGION 1..7: note = Description of Artificial Sequence: Synthetic peptide
LVSELDS

SEQ ID NO: 539 - protein, 9 aa, synthetic construct
  REGION 1..9: note = Description of Artificial Sequence: Synthetic peptide
WQGTHFPRT

SEQ ID NO: 540 - protein, 117 aa, synthetic construct
  REGION 1..117: note = Description of Artificial Sequence: Synthetic polypeptide
QVQLVQSGAEVKKPGASVKVSCKASGYSFSGYFMNWVRQAPGQGLEWMGLINPYNGDSFYNQKFKGRVTMTRQTSTSTVYMELSSLRSEDTAVYYCVRGLRRDFDYWGQGTLVTVSS

SEQ ID NO: 541 - protein, 112 aa, synthetic construct
  REGION 1..112: note = Description of Artificial Sequence: Synthetic polypeptide
DVVMTQSPLSLPVTLGQPASISCKSSQSLLDSDGKTYLNWLFQRPGQSPRRLIYLVSELDSGVPDRFSGSGSGTDFTLKISRVEAEDVGVYYCWQGTHFPRTFGGGTKLEIK

SEQ ID NO: 542 - protein, 5 aa, synthetic construct
  REGION 1..5: note = Description of Artificial Sequence: Synthetic peptide
DFGMN

SEQ ID NO: 543 - protein, 17 aa, synthetic construct
  REGION 1..17: note = Description of Artificial Sequence: Synthetic peptide
WINTFTGEPSYGNVFKG

SEQ ID NO: 544 - protein, 10 aa, synthetic construct
  REGION 1..10: note = Description of Artificial Sequence: Synthetic peptide
RHGNGNVFDS

SEQ ID NO: 545 - protein, 11 aa, synthetic construct
  REGION 1..11: note = Description of Artificial Sequence: Synthetic peptide
RASQSIGSNIH

SEQ ID NO: 546 - protein, 7 aa, synthetic construct
  REGION 1..7: note = Description of Artificial Sequence: Synthetic peptide
YTSESIS

SEQ ID NO: 547 - protein, 9 aa, synthetic construct
  REGION 1..9: note = Description of Artificial Sequence: Synthetic peptide
QQSNSWPLT

SEQ ID NO: 548 - protein, 119 aa, synthetic construct
  REGION 1..119: note = Description of Artificial Sequence: Synthetic polypeptide
QVQLVQSGSELKKPGASVKVSCKASGYTFTDFGMNWVRQAPGQGLEWMGWINTFTGEPSYGNVFKGRFVFSLDTSVSTAYLQISSLKAEDTAVYYCARRHGNGNVFDSWGQGTLVTVSS

SEQ ID NO: 549 - protein, 108 aa, synthetic construct
  REGION 1..108: note = Description of Artificial Sequence: Synthetic polypeptide
EIVLTQSPDFQSVTPKEKVTITCRASQSIGSNIHWYQQKPDQSPKLLIKYTSESISGVPSRFSGSGSGTDFTLTINSLEAEDAATYYCQQSNSWPLTFGGGTKVEIKR

SEQ ID NO: 550 - protein, 5 aa, synthetic construct
  REGION 1..5: note = Description of Artificial Sequence: Synthetic peptide
TAAIS

SEQ ID NO: 551 - protein, 17 aa, synthetic construct
  REGION 1..17: note = Description of Artificial Sequence: Synthetic peptide
GIIPIFGKAHYAQKFQG

SEQ ID NO: 552 - protein, 14 aa, synthetic construct
  REGION 1..14: note = Description of Artificial Sequence: Synthetic peptide
KFHFVSGSPFGMDV

SEQ ID NO: 553 - protein, 11 aa, synthetic construct
  REGION 1..11: note = Description of Artificial Sequence: Synthetic peptide
RASQSVSSYLA

SEQ ID NO: 554 - protein, 7 aa, synthetic construct
  REGION 1..7: note = Description of Artificial Sequence: Synthetic peptide
DASNRAT

SEQ ID NO: 555 - protein, 8 aa, synthetic construct
  REGION 1..8: note = Description of Artificial Sequence: Synthetic peptide
QQRSNWPT

SEQ ID NO: 556 - protein, 123 aa, synthetic construct
  REGION 1..123: note = Description of Artificial Sequence: Synthetic polypeptide
QVQLVQSGAEVKKPGSSVKVSCKTSGDTFSTAAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 557 - protein, 106 aa, synthetic construct
  REGION 1..106: note = Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 558 - protein, 5 aa, synthetic construct
  REGION 1..5: note = Description of Artificial Sequence: Synthetic peptide
SYAIS

SEQ ID NO: 559 - protein, 17 aa, synthetic construct
  REGION 1..17: note = Description of Artificial Sequence: Synthetic peptide
GIIPIFGTANYAQKFQG

SEQ ID NO: 560 - protein, 21 aa, synthetic construct
  REGION 1..21: note = Description of Artificial Sequence: Synthetic peptide
APLRFLEWSTQDHYYYYYMDV

SEQ ID NO: 561 - protein, 11 aa, synthetic construct
  REGION 1..11: note = Description of Artificial Sequence: Synthetic peptide
QGDSLRSYYAT

SEQ ID NO: 562 - protein, 7 aa, synthetic construct
  REGION 1..7: note = Description of Artificial Sequence: Synthetic peptide
GENKRPS

SEQ ID NO: 563 - protein, 11 aa, synthetic construct
  REGION 1..11: note = Description of Artificial Sequence: Synthetic peptide
KSRDGSGQHLV

SEQ ID NO: 564 - protein, 130 aa, synthetic construct
  REGION 1..130: note = Description of Artificial Sequence: Synthetic polypeptide
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARAPLRFLEWSTQDHYYYYYMDVWGKGTTVTVSS

SEQ ID NO: 565 - protein, 108 aa, synthetic construct
  REGION 1..108: note = Description of Artificial Sequence: Synthetic polypeptide
SSELTQDPAVSVALGQTVRITCQGDSLRSYYATWYQQKPGQAPILVIYGENKRPSGIPDRFSGSSSGNTASLTITGAQAEDEADYYCKSRDGSGQHLVFGGGTKLTVL

SEQ ID NO: 566 - protein, 5 aa, synthetic construct
  REGION 1..5: note = Description of Artificial Sequence: Synthetic peptide
SYWIN

SEQ ID NO: 567 - protein, 17 aa, synthetic construct
  REGION 1..17: note = Description of Artificial Sequence: Synthetic peptide
NIYPSDSYTNYNQKFKD

SEQ ID NO: 568 - protein, 9 aa, synthetic construct
  REGION 1..9: note = Description of Artificial Sequence: Synthetic peptide
SWRGNSFDY

SEQ ID NO: 569 - protein, 17 aa, synthetic construct
  REGION 1..17: note = Description of Artificial Sequence: Synthetic peptide
KSSQSLLNSGNQKNYLT

SEQ ID NO: 570 - protein, 7 aa, synthetic construct
  REGION 1..7: note = Description of Artificial Sequence: Synthetic peptide
WASTRES

SEQ ID NO: 571 - protein, 9 aa, synthetic construct
  REGION 1..9: note = Description of Artificial Sequence: Synthetic peptide
QNDYSYPFT

SEQ ID NO: 572 - protein, 118 aa, synthetic construct
  REGION 1..118: note = Description of Artificial Sequence: Synthetic polypeptide
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSS

SEQ ID NO: 573 - protein, 113 aa, synthetic construct
  REGION 1..113: note = Description of Artificial Sequence: Synthetic polypeptide
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 574 - protein, 5 aa, synthetic construct
  REGION 1..5: note = Description of Artificial Sequence: Synthetic peptide
NYGMN

SEQ ID NO: 575 - protein, 17 aa, synthetic construct
  REGION 1..17: note = Description of Artificial Sequence: Synthetic peptide
WINTNTGEPTYAEEFKG

SEQ ID NO: 576 - protein, 9 aa, synthetic construct
  REGION 1..9: note = Description of Artificial Sequence: Synthetic peptide
LGFGNAMDY

SEQ ID NO: 577 - protein, 17 aa, synthetic construct
  REGION 1..17: note = Description of Artificial Sequence: Synthetic peptide
KSSQSLLNSGNQKNYLT

SEQ ID NO: 578 - protein, 7 aa, synthetic construct
  REGION 1..7: note = Description of Artificial Sequence: Synthetic peptide
WASTRES

SEQ ID NO: 579 - protein, 9 aa, synthetic construct
  REGION 1..9: note = Description of Artificial Sequence: Synthetic peptide
QNDYSYPLT

SEQ ID NO: 580 - protein, 118 aa, synthetic construct
  REGION 1..118: note = Description of Artificial Sequence: Synthetic polypeptide
QIQLVQSGPELKKPGETVKISCKASGYTFTNYGMNWVKQAPGKGLKWMGWINTNTGEPTYAEEFKGRFAFSLETSASTAYLQINNLKNEDTATYFCARLGFGNAMDYWGQGTSVTVSS

SEQ ID NO: 581 - protein, 113 aa, synthetic construct
  REGION 1..113: note = Description of Artificial Sequence: Synthetic polypeptide
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELK

SEQ ID NO: 582 - protein, 5 aa, synthetic construct
  REGION 1..5: note = Description of Artificial Sequence: Synthetic peptide
SYNMN

SEQ ID NO: 583 - protein, 17 aa, synthetic construct
  REGION 1..17: note = Description of Artificial Sequence: Synthetic peptide
YISSSSSTIYYADSVKG

SEQ ID NO: 584 - protein, 8 aa, synthetic construct
  REGION 1..8: note = Description of Artificial Sequence: Synthetic peptide
AYYYGMDV

SEQ ID NO: 585 - protein, 11 aa, synthetic construct
  REGION 1..11: note = Description of Artificial Sequence: Synthetic peptide
RASQGISGWLA

SEQ ID NO: 586 - protein, 7 aa, synthetic construct
  REGION 1..7: note = Description of Artificial Sequence: Synthetic peptide
AASTLQS

SEQ ID NO: 587 - protein, 9 aa, synthetic construct
  REGION 1..9: note = Description of Artificial Sequence: Synthetic peptide
QQANSFPPT

SEQ ID NO: 588 - protein, 117 aa, synthetic construct
  REGION 1..117: note = Description of Artificial Sequence: Synthetic polypeptide
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSS

SEQ ID NO: 589 - protein, 107 aa, synthetic construct
  REGION 1..107: note = Description of Artificial Sequence: Synthetic polypeptide
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIK

SEQ ID NO: 590 - protein, 5 aa, synthetic construct
  REGION 1..5: note = Description of Artificial Sequence: Synthetic peptide
SYGMH

SEQ ID NO: 591 - protein, 17 aa, synthetic construct
  REGION 1..17: note = Description of Artificial Sequence: Synthetic peptide
VIWYDGSNQYYADSVKG

SEQ ID NO: 592 - protein, 11 aa, synthetic construct
  REGION 1..11: note = Description of Artificial Sequence: Synthetic peptide
GLTSGRYGMDV

SEQ ID NO: 593 - protein, 16 aa, synthetic construct
  REGION 1..16: note = Description of Artificial Sequence: Synthetic peptide
RSSQSLLLSHGFNYLD

SEQ ID NO: 594 - protein, 7 aa, synthetic construct
  REGION 1..7: note = Description of Artificial Sequence: Synthetic peptide
LGSSRAS

SEQ ID NO: 595 - protein, 9 aa, synthetic construct
  REGION 1..9: note = Description of Artificial Sequence: Synthetic peptide
MQPLQIPWT

SEQ ID NO: 596 - protein, 120 aa, synthetic construct
  REGION 1..120: note = Description of Artificial Sequence: Synthetic polypeptide
QVQLVESGGGVVQPGRSLRLSCAASGFTFSSYGMHWVRQAPGKGLEWVAVIWYDGSNQYYADSVKGRFTISRDNSKNTLFLQMHSLRAEDTAVYYCARGLTSGRYGMDVWGQGTTVTVSS

SEQ ID NO: 597 - protein, 112 aa, synthetic construct
  REGION 1..112: note = Description of Artificial Sequence: Synthetic polypeptide
DIVMTQSPLSLPVTPGEPASISCRSSQSLLLSHGFNYLDWYLQKPGQSPQLLIYLGSSRASGVPDRFSGSGSGTDFTLKISRVEAEDVGLYYCMQPLQIPWTFGQGTKVEIK

SEQ ID NO: 598 - protein, 5 aa, synthetic construct
  REGION 1..5: note = Description of Artificial Sequence: Synthetic peptide
SGYWN

SEQ ID NO: 599 - protein, 16 aa, synthetic construct
  REGION 1..16: note = Description of Artificial Sequence: Synthetic peptide
YISDSGITYYNPSLKS

SEQ ID NO: 600 - protein, 11 aa, synthetic construct
  REGION 1..11: note = Description of Artificial Sequence: Synthetic peptide
RTLATYYAMDY

SEQ ID NO: 601 - protein, 16 aa, synthetic construct
  REGION 1..16: note = Description of Artificial Sequence: Synthetic peptide
RASQSLVHSDGNTYLH

SEQ ID NO: 602 - protein, 7 aa, synthetic construct
  REGION 1..7: note = Description of Artificial Sequence: Synthetic peptide
RVSNRFS

SEQ ID NO: 603 - protein, 9 aa, synthetic construct
  REGION 1..9: note = Description of Artificial Sequence: Synthetic peptide
SQSTHVPPT

SEQ ID NO: 604 - protein, 119 aa, synthetic construct
  REGION 1..119: note = Description of Artificial Sequence: Synthetic polypeptide
QVQLQESGPGLVKPSETLSLTCTVSGDSITSGYWNWIRQPPGKGLEYIGYISDSGITYYNPSLKSRVTISRDTSKNQYSLKLSSVTAADTAVYYCARRTLATYYAMDYWGQGTLVTVSS

SEQ ID NO: 605 - protein, 113 aa, synthetic construct
  REGION 1..113: note = Description of Artificial Sequence: Synthetic polypeptide
DFVMTQSPLSLPVTLGQPASISCRASQSLVHSDGNTYLHWYQQRPGQSPRLLIYRVSNRFSGVPDRFSGSGSGTDFTLKISRVEAEDVGVYYCSQSTHVPPTFGQGTKLEIKR

SEQ ID NO: 606 - protein, 5 aa, synthetic construct
  REGION 1..5: note = Description of Artificial Sequence: Synthetic peptide
NYAMS

SEQ ID NO: 607 - protein, 17 aa, synthetic construct
  REGION 1..17: note = Description of Artificial Sequence: Synthetic peptide
SISGSGDYTYYTDSVKG

SEQ ID NO: 608 - protein, 9 aa, synthetic construct
  REGION 1..9: note = Description of Artificial Sequence: Synthetic peptide
SPWGYYLDS

SEQ ID NO: 609 - protein, 11 aa, synthetic construct
  REGION 1..11: note = Description of Artificial Sequence: Synthetic peptide
RASQGISSRLA

SEQ ID NO: 610 - protein, 7 aa, synthetic construct
  REGION 1..7: note = Description of Artificial Sequence: Synthetic peptide
AASSLQS

SEQ ID NO: 611 - protein, 9 aa, synthetic construct
  REGION 1..9: note = Description of Artificial Sequence: Synthetic peptide
QQYNSYPYT

SEQ ID NO: 612 - protein, 118 aa, synthetic construct
  REGION 1..118: note = Description of Artificial Sequence: Synthetic polypeptide
EVQLLESGGGLVQPGGSLRLSCAASGFTFSNYAMSWVRQAPGKGLEWVSSISGSGDYTYYTDSVKGRFTISRDNSKNTLYLQMNSLRAEDTAVYYCARSPWGYYLDSWGQGTLVTVSS

SEQ ID NO: 613 - protein, 107 aa, synthetic construct
  REGION 1..107: note = Description of Artificial Sequence: Synthetic polypeptide
DIQMTQSPPSLSASAGDRVTITCRASQGISSRLAWYQQKPEKAPKSLIYAASSLQSGVPSRFSGSGSGTDFTLTISSLQPEDFATYYCQQYNSYPYTFGQGTKLEIK

SEQ ID NO: 614 - protein, 5 aa, synthetic construct
  REGION 1..5: note = Description of Artificial Sequence: Synthetic peptide
DHAIH

SEQ ID NO: 615 - protein, 17 aa, synthetic construct
  REGION 1..17: note = Description of Artificial Sequence: Synthetic peptide
YFSPGNDDIKYNEKFRG

SEQ ID NO: 616 - protein, 6 aa, synthetic construct
  REGION 1..6: note = Description of Artificial Sequence: Synthetic peptide
SLSTPY

SEQ ID NO: 617 - protein, 17 aa, synthetic construct
  REGION 1..17: note = Description of Artificial Sequence: Synthetic peptide
KSSQSLLNRGNHKNYLT

SEQ ID NO: 618 - protein, 7 aa, synthetic construct
  REGION 1..7: note = Description of Artificial Sequence: Synthetic peptide
WASTRES

SEQ ID NO: 619 - protein, 9 aa, synthetic construct
  REGION 1..9: note = Description of Artificial Sequence: Synthetic peptide
QNDYTYPYT

SEQ ID NO: 620 - protein, 115 aa, synthetic construct
  REGION 1..115: note = Description of Artificial Sequence: Synthetic polypeptide
EVQLVQSGAEVKKPGASVKVSCKASGYTFTDHAIHWVRQAPGQGLEWMGYFSPGNDDIKYNEKFRGRVTMTADKSSSTAYMELRSLRSDDTAVYFCKRSLSTPYWGQGTLVTVSS

SEQ ID NO: 621 - protein, 113 aa, synthetic construct
  REGION 1..113: note = Description of Artificial Sequence: Synthetic polypeptide
DIVMTQSPDSLAVSLGERATINCKSSQSLLNRGNHKNYLTWYQQKPGQPPKLLIYWASTRESGVPDRFSGSGSGTDFTLTISSLQAEDVAVYYCQNDYTYPYTFGQGTKVEIK

SEQ ID NO: 622 - protein, 5 aa, synthetic construct
  REGION 1..5: note = Description of Artificial Sequence: Synthetic peptide
SYNMH

SEQ ID NO: 623 - protein, 17 aa, synthetic construct
  REGION 1..17: note = Description of Artificial Sequence: Synthetic peptide
AIYPGNGDTSYNQKFKG

SEQ ID NO: 624 - protein, 12 aa, synthetic construct
  REGION 1..12: note = Description of Artificial Sequence: Synthetic peptide
STYYGGDWYFNV

SEQ ID NO: 625 - protein, 10 aa, synthetic construct
  REGION 1..10: note = Description of Artificial Sequence: Synthetic peptide
RASSSVSYIH

SEQ ID NO: 626 - protein, 7 aa, synthetic construct
  REGION 1..7: note = Description of Artificial Sequence: Synthetic peptide
ATSNLAS

SEQ ID NO: 627 - protein, 9 aa, synthetic construct
  REGION 1..9: note = Description of Artificial Sequence: Synthetic peptide
QQWTSNPPT

SEQ ID NO: 628 - protein, 121 aa, synthetic construct [sequence omitted]

SEQ ID NO: 629 - protein, 106 aa, synthetic construct [sequence omitted]

SEQ ID NO: 630 - protein, 5 aa, synthetic construct
  REGION 1..5: note = Description of Artificial Sequence: Synthetic peptide
DTYIH

SEQ ID NO: 631 - protein, 17 aa, synthetic construct
  REGION 1..17: note = Description of Artificial Sequence: Synthetic peptide
RIYPTNGYTRYADSVKG

SEQ ID NO: 632 - protein, 11 aa, synthetic construct
  REGION 1..11: note = Description of Artificial Sequence: Synthetic peptide
WGGDGFYAMDY

SEQ ID NO: 633 - protein, 11 aa, synthetic construct
  REGION 1..11: note = Description of Artificial Sequence: Synthetic peptide
RASQDVNTAVA

SEQ ID NO: 634 - protein, 7 aa, synthetic construct
  REGION 1..7: note = Description of Artificial Sequence: Synthetic peptide
SASFLYS

SEQ ID NO: 635 - protein, 9 aa, synthetic construct
  REGION 1..9: note = Description of Artificial Sequence: Synthetic peptide
QQHYTTPPT

SEQ ID NO: 636 - protein, 120 aa, synthetic construct [sequence omitted]

SEQ ID NO: 637 - protein, 107 aa, synthetic construct [sequence omitted]

SEQ ID NO: 638 - protein, 5 aa, synthetic construct
  REGION 1..5: note = Description of Artificial Sequence: Synthetic peptide
SYWIE

SEQ ID NO: 639 - protein, 17 aa, synthetic construct
  REGION 1..17: note = Description of Artificial Sequence: Synthetic peptide
EILPGGGDTNYNEIFKG

SEQ ID NO: 640 - protein, 8 aa, synthetic construct
  REGION 1..8: note = Description of Artificial Sequence: Synthetic peptide
RVPIRLDY

SEQ ID NO: 641 - protein, 15 aa, synthetic construct
  REGION 1..15: note = Description of Artificial Sequence: Synthetic peptide
KASQSVDYEGDSFLN

SEQ ID NO: 642 - protein, 7 aa, synthetic construct
  REGION 1..7: note = Description of Artificial Sequence: Synthetic peptide
AASNLES

SEQ ID NO: 643 - protein, 9 aa, synthetic construct
  REGION 1..9: note = Description of Artificial Sequence: Synthetic peptide
QQSNEDPLT

SEQ ID NO: 644 - protein, 117 aa, synthetic construct [sequence omitted]

SEQ ID NO: 645 - protein, 111 aa, synthetic construct [sequence omitted]

SEQ ID NO: 646 - protein, 5 aa, synthetic construct
  REGION 1..5: note = Description of Artificial Sequence: Synthetic peptide
DFAMS

SEQ ID NO: 647 - protein, 17 aa, synthetic construct
  REGION 1..17: note = Description of Artificial Sequence: Synthetic peptide
TIGRVAFHTYYPDSMKG

SEQ ID NO: 648 - protein, 11 aa, synthetic construct
  REGION 1..11: note = Description of Artificial Sequence: Synthetic peptide
HRGFDVGHFDF

SEQ ID NO: 649 - protein, 16 aa, synthetic construct
  REGION 1..16: note = Description of Artificial Sequence: Synthetic peptide
RSSETLVHSSGNTYLE

SEQ ID NO: 650 - protein, 7 aa, synthetic construct
  REGION 1..7: note = Description of Artificial Sequence: Synthetic peptide
RVSNRFS

SEQ ID NO: 651 - protein, 9 aa, synthetic construct
  REGION 1..9: note = Description of Artificial Sequence: Synthetic peptide
FQGSFNPLT

SEQ ID NO: 652 - protein, 120 aa, synthetic construct
  REGION 1..120: note = Description of Artificial Sequence: Synthetic polypeptide
EVQLVESGGGLVQPGGSLRLSCAASGFSFSDFAMSWVRQAPGKGLEWVATIGRVAFHTYYPDSMKGRFTISRDNSKNTLYLQMNSLRAEDTAVYYCARHRGFDVGHFDFWGQGTLVTVSS

SEQ ID NO: 653 - protein, 112 aa, synthetic construct
  REGION 1..112: note = Description of Artificial Sequence: Synthetic polypeptide
DIQMTQSPSSLSASVGDRVTITCRSSETLVHSSGNTYLEWYQQKPGKAPKLLIYRVSNRFSGVPSRFSGSGSGTDFTLTISSLQPEDFATYYCFQGSFNPLTFGQGTKVEIK

SEQ ID NO: 654 - protein, 6 aa, synthetic construct
  REGION 1..6: note = Description of Artificial Sequence: Synthetic peptide
NDYAWN

SEQ ID NO: 655 - protein, 16 aa, synthetic construct
  REGION 1..16: note = Description of Artificial Sequence: Synthetic peptide
YISYSGYTTYNPSLKS

SEQ ID NO: 656 - protein, 7 aa, synthetic construct
  REGION 1..7: note = Description of Artificial Sequence: Synthetic peptide
WTSGLDY

SEQ ID NO: 657 - protein, 11 aa, synthetic construct
  REGION 1..11: note = Description of Artificial Sequence: Synthetic peptide
KASDLIHNWLA

SEQ ID NO: 658 - protein, 7 aa, synthetic construct
  REGION 1..7: note = Description of Artificial Sequence: Synthetic peptide
GATSLET

SEQ ID NO: 659 - protein, 9 aa, synthetic construct
  REGION 1..9: note = Description of Artificial Sequence: Synthetic peptide
QQYWTTPFT

SEQ ID NO: 660 - protein, 116 aa, synthetic construct
  REGION 1..116: note = Description of Artificial Sequence: Synthetic polypeptide
EVQLVESGGGLVQPGGSLRLSCAASGYSITNDYAWNWVRQAPGKGLEWVGYISYSGYTTYNPSLKSRFTISRDTSKNTLYLQMNSLRAEDTAVYYCARWTSGLDYWGQGTLVTVSS

SEQ ID NO: 661 - protein, 107 aa, synthetic construct
  REGION 1..107: note = Description of Artificial Sequence: Synthetic polypeptide
DIQMTQSPSSLSASVGDRVTITCKASDLIHNWLAWYQQKPGKAPKLLIYGATSLETGVPSRFSGSGSGTDFTLTISSLQPEDFATYYCQQYWTTPFTFGQGTKVEIK

SEQ ID NO: 662 - protein, 6 aa, synthetic construct
  REGION 1..6: note = Description of Artificial Sequence: Synthetic peptide
SDYAWN

SEQ ID NO: 663 - protein, 16 aa, synthetic construct
  REGION 1..16: note = Description of Artificial Sequence: Synthetic peptide
YISNSGSTSYNPSLKS

SEQ ID NO: 664 - protein, 15 aa, synthetic construct
  REGION 1..15: note = Description of Artificial Sequence: Synthetic peptide
ERNYDYDDYYYAMDY

SEQ ID NO: 665 - protein, 17 aa, synthetic construct
  REGION 1..17: note = Description of Artificial Sequence: Synthetic peptide
KSSQSLLYRSNQKNYLA

SEQ ID NO: 666 - protein, 7 aa, synthetic construct
  REGION 1..7: note = Description of Artificial Sequence: Synthetic peptide
WASTRES

SEQ ID NO: 667 - protein, 9 aa, synthetic construct
  REGION 1..9: note = Description of Artificial Sequence: Synthetic peptide
QQYYNYPRT

SEQ ID NO: 668 - protein, 124 aa, synthetic construct
  REGION 1..124: note = Description of Artificial Sequence: Synthetic polypeptide
EVQLVESGGGLVQPGGSLRLSCAVSGYSITSDYAWNWVRQAPGKGLEWVGYISNSGSTSYNPSLKSRFTISRDTSKNTLYLQMNSLRAEDTAVYYCARERNYDYDDYYYAMDYWGQGTLVTVSS

SEQ ID NO: 669 - protein, 113 aa, synthetic construct
  REGION 1..113: note = Description of Artificial Sequence: Synthetic polypeptide
DIQMTQSPSSLSASVGDRVTITCKSSQSLLYRSNQKNYLAWYQQKPGKAPKLLIYWASTRESGVPSRFSGSGSGTDFTLTISSLQPEDFATYYCQQYYNYPRTFGQGTKVEIK

SEQ ID NO: 670 - protein, 5 aa, synthetic construct
  REGION 1..5: note = Description of Artificial Sequence: Synthetic peptide
NYWMH

SEQ ID NO: 671 - protein, 17 aa, synthetic construct
  REGION 1..17: note = Description of Artificial Sequence: Synthetic peptide
ATYRGHSDTYYNQKFKG

SEQ ID NO: 672 - protein, 12 aa, synthetic construct
  REGION 1..12: note = Description of Artificial Sequence: Synthetic peptide
GAIYDGYDVLDN

SEQ ID NO: 673 - protein, 11 aa, synthetic construct
  REGION 1..11: note = Description of Artificial Sequence: Synthetic peptide
SASQDISNYLN

SEQ ID NO: 674 - protein, 7 aa, synthetic construct
  REGION 1..7: note = Description of Artificial Sequence: Synthetic peptide
YTSNLHS

SEQ ID NO: 675 - protein, 9 aa, synthetic construct
  REGION 1..9: note = Description of Artificial Sequence: Synthetic peptide
QQYRKLPWT

SEQ ID NO: 676 - protein, 121 aa, synthetic construct
  REGION 1..121: note = Description of Artificial Sequence: Synthetic polypeptide
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 677 - protein, 107 aa, synthetic construct
  REGION 1..107: note = Description of Artificial Sequence: Synthetic polypeptide
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIK

SEQ ID NO: 678 - protein, 5 aa, synthetic construct
  REGION 1..5: note = Description of Artificial Sequence: Synthetic peptide
AYTMH

SEQ ID NO: 679 - protein, 17 aa, synthetic construct
  REGION 1..17: note = Description of Artificial Sequence: Synthetic peptide
WIKPNNGLANYAQKFQG

SEQ ID NO: 680 - protein, 9 aa, synthetic construct
  REGION 1..9: note = Description of Artificial Sequence: Synthetic peptide
SEITTEFDY

SEQ ID NO: 681 - protein, 15 aa, synthetic construct
  REGION 1..15: note = Description of Artificial Sequence: Synthetic peptide
KSSESVDSYANSFLH

SEQ ID NO: 682 - protein, 7 aa, synthetic construct
  REGION 1..7: note = Description of Artificial Sequence: Synthetic peptide
RASTRES

SEQ ID NO: 683 - protein, 9 aa, synthetic construct
  REGION 1..9: note = Description of Artificial Sequence: Synthetic peptide
QQSKEDPLT

SEQ ID NO: 684 - protein, 118 aa, synthetic construct
  REGION 1..118: note = Description of Artificial Sequence: Synthetic polypeptide
QVQLVQSGAEVKKPGASVKVSCKASGYIFTAYTMHWVRQAPGQGLEWMGWIKPNNGLANYAQKFQGRVTMTRDTSISTAYMELSRLRSDDTAVYYCARSEITTEFDYWGQGTLVTVSS

SEQ ID NO: 685 - protein, 111 aa, synthetic construct
  REGION 1..111: note = Description of Artificial Sequence: Synthetic polypeptide
DIVMTQSPDSLAVSLGERATINCKSSESVDSYANSFLHWYQQKPGQPPKLLIYRASTRESGVPDRFSGSGSGTDFTLTISSLQAEDVAVYYCQQSKEDPLTFGGGTKVEIK

SEQ ID NO: 686 - protein, 6 aa, synthetic construct
  REGION 1..6: note = Description of Artificial Sequence: Synthetic peptide
SDFAWN

SEQ ID NO: 687 - protein, 16 aa, synthetic construct
  REGION 1..16: note = Description of Artificial Sequence: Synthetic peptide
YISYSGNTRYQPSLKS

SEQ ID NO: 688 - protein, 7 aa, synthetic construct
  REGION 1..7: note = Description of Artificial Sequence: Synthetic peptide
AGRGFPY

SEQ ID NO: 689 - protein, 11 aa, synthetic construct
  REGION 1..11: note = Description of Artificial Sequence: Synthetic peptide
HSSQDINSNIG

SEQ ID NO: 690 - protein, 7 aa, synthetic construct
  REGION 1..7: note = Description of Artificial Sequence: Synthetic peptide
HGTNLDD

SEQ ID NO: 691 - protein, 9 aa, synthetic construct
  REGION 1..9: note = Description of Artificial Sequence: Synthetic peptide
VQYAQFPWT

SEQ ID NO: 692 - protein, 116 aa, synthetic construct
  REGION 1..116: note = Description of Artificial Sequence: Synthetic polypeptide
QVQLQESGPGLVKPSQTLSLTCTVSGYSISSDFAWNWIRQPPGKGLEWMGYISYSGNTRYQPSLKSRITISRDTSKNQFFLKLNSVTAADTATYYCVTAGRGFPYWGQGTLVTVSS

SEQ ID NO: 693 - protein, 107 aa, synthetic construct; identical to SEQ ID NO: 319
  REGION 1..107: note = Description of Artificial Sequence: Synthetic polypeptide

SEQ ID NO: 694 - protein, 5 aa, synthetic construct
  REGION 1..5: note = Description of Artificial Sequence: Synthetic peptide
DYYMA

SEQ ID NO: 695 - protein, 17 aa, synthetic construct
  REGION 1..17: note = Description of Artificial Sequence: Synthetic peptide
SINYDGSSTYYVDSVKG

SEQ ID NO: 696 - protein, 8 aa, synthetic construct
  REGION 1..8: note = Description of Artificial Sequence: Synthetic peptide
DRGYYFDY

SEQ ID NO: 697 - protein, 16 aa, synthetic construct
  REGION 1..16: note = Description of Artificial Sequence: Synthetic peptide
RSSQSLVHSNGNTYLH

SEQ ID NO: 698 - protein, 7 aa, synthetic construct
  REGION 1..7: note = Description of Artificial Sequence: Synthetic peptide
KVSNRFS

SEQ ID NO: 699 - protein, 10 aa, synthetic construct
  REGION 1..10: note = Description of Artificial Sequence: Synthetic peptide
SQSTHVPPFT

SEQ ID NO: 700 - protein, 117 aa, synthetic construct
  REGION 1..117: note = Description of Artificial Sequence: Synthetic polypeptide
EVQLVESGGGLVQPGGSLRLSCAASGFTFSDYYMAWVRQAPGKGLEWVASINYDGSSTYYVDSVKGRFTISRDNAKNSLYLQMNSLRAEDTAVYYCARDRGYYFDYWGQGTTVTVSS

SEQ ID NO: 701 - protein, 113 aa, synthetic construct
  REGION 1..113: note = Description of Artificial Sequence: Synthetic polypeptide
DVVMTQTPLSLSVTPGQPASISCRSSQSLVHSNGNTYLHWYLQKPGQSPQLLIYKVSNRFSGVPDRFSGSGSGTDFTLKISRVEAEDVGVYFCSQSTHVPPFTFGGGTKVEIK

SEQ ID NO: 702 - protein, 5 aa, synthetic construct
  REGION 1..5: note = Description of Artificial Sequence: Synthetic peptide
SYGMH

SEQ ID NO: 703 - protein, 17 aa, synthetic construct
  REGION 1..17: note = Description of Artificial Sequence: Synthetic peptide
VIWYDGSNQYYADSVKG

SEQ ID NO: 704 - protein, 11 aa, synthetic construct
  REGION 1..11: note = Description of Artificial Sequence: Synthetic peptide
GLTSGRYGMDV

SEQ ID NO: 705 - protein, 16 aa, synthetic construct
  REGION 1..16: note = Description of Artificial Sequence: Synthetic peptide
RSSQSLLLSHGFNYLD

SEQ ID NO: 706 - protein, 7 aa, synthetic construct
  REGION 1..7: note = Description of Artificial Sequence: Synthetic peptide
LGSSRAS

SEQ ID NO: 707 - protein, 9 aa, synthetic construct
  REGION 1..9: note = Description of Artificial Sequence: Synthetic peptide
MQPLQIPWT

SEQ ID NO: 708 - protein, 120 aa, synthetic construct; identical to SEQ ID NO: 596
  REGION 1..120: note = Description of Artificial Sequence: Synthetic polypeptide

SEQ ID NO: 709 - protein, 112 aa, synthetic construct; identical to SEQ ID NO: 597
  REGION 1..112: note = Description of Artificial Sequence: Synthetic polypeptide

SEQ ID NO: 710 - protein, 5 aa, synthetic construct
  REGION 1..5: note = Description of Artificial Sequence: Synthetic peptide
NAWMS

SEQ ID NO: 711 - protein, 17 aa, synthetic construct
  REGION 1..17: note = Description of Artificial Sequence: Synthetic peptide
YISSSGSTIYYADSVKG

SEQ ID NO: 712 - protein, 8 aa, synthetic construct
  REGION 1..8: note = Description of Artificial Sequence: Synthetic peptide
EGLWAFDY

SEQ ID NO: 713 - protein, 14 aa, synthetic construct
  REGION 1..14: note = Description of Artificial Sequence: Synthetic peptide
TGSSSNIGAGYVVH

SEQ ID NO: 714 - protein, 7 aa, synthetic construct
  REGION 1..7: note = Description of Artificial Sequence: Synthetic peptide
DNNKRPS

SEQ ID NO: 715 - protein, 11 aa, synthetic construct
  REGION 1..11: note = Description of Artificial Sequence: Synthetic peptide
AAWDDRLNGPV

SEQ ID NO: 716 - protein, 117 aa, synthetic construct
  REGION 1..117: note = Description of Artificial Sequence: Synthetic polypeptide
EVQLLESGGGLVQPGGSLRLSCAASGFTFSNAWMSWVRQAPGKGLEWVSYISSSGSTIYYADSVKGRFTISRDNSKNTLYLQMNSLRAEDTAVYYCAREGLWAFDYWGQGTLVTVSS

SEQ ID NO: 717 - protein, 111 aa, synthetic construct
  REGION 1..111: note = Description of Artificial Sequence: Synthetic polypeptide
ESVLTQPPSVSGAPGQRVTISCTGSSSNIGAGYVVHWYQQLPGTAPKLLIYDNNKRPSGVPDRFSGSKSGTSASLAISGLRSEDEADYYCAAWDDRLNGPVFGGGTKLTVL

SEQ ID NO: 718 - protein, 5 aa, synthetic construct
  REGION 1..5: note = Description of Artificial Sequence: Synthetic peptide
GYYWS

SEQ ID NO: 719 - protein, 16 aa, synthetic construct
  REGION 1..16: note = Description of Artificial Sequence: Synthetic peptide
EINHRGNTNDNPSLKS

SEQ ID NO: 720 - protein, 11 aa, synthetic construct
  REGION 1..11: note = Description of Artificial Sequence: Synthetic peptide
ERGYTYGNFDH

SEQ ID NO: 721 - protein, 11 aa, synthetic construct
  REGION 1..11: note = Description of Artificial Sequence: Synthetic peptide
RASQSVSRNLA

SEQ ID NO: 722 - protein, 7 aa, synthetic construct
  REGION 1..7: note = Description of Artificial Sequence: Synthetic peptide
GASTRAT

SEQ ID NO: 723 - protein, 9 aa, synthetic construct
  REGION 1..9: note = Description of Artificial Sequence: Synthetic peptide
QQYKTWPRT

SEQ ID NO: 724 - protein, 119 aa, synthetic construct
  REGION 1..119: note = Description of Artificial Sequence: Synthetic polypeptide
QVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSS

SEQ ID NO: 725 - protein, 107 aa, synthetic construct
  REGION 1..107: note = Description of Artificial Sequence: Synthetic polypeptide
EIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIK

SEQ ID NO: 726 - protein, 5 aa, synthetic construct
  REGION 1..5: note = Description of Artificial Sequence: Synthetic peptide
SYAMN

SEQ ID NO: 727 - protein, 17 aa, synthetic construct
  REGION 1..17: note = Description of Artificial Sequence: Synthetic peptide
TTSGSGASTYYADSVKG

SEQ ID NO: 728 - protein, 7 aa, synthetic construct
  REGION 1..7: note = Description of Artificial Sequence: Synthetic peptide
IWIAFDI

SEQ ID NO: 729 - protein, 12 aa, synthetic construct
  REGION 1..12: note = Description of Artificial Sequence: Synthetic peptide
RASQSVSSSYLA

SEQ ID NO: 730 - protein, 7 aa, synthetic construct
  REGION 1..7: note = Description of Artificial Sequence: Synthetic peptide
GASSRAT

SEQ ID NO: 731 - protein, 9 aa, synthetic construct
  REGION 1..9: note = Description of Artificial Sequence: Synthetic peptide
QQYGSSPYT

SEQ ID NO: 732 - protein, 116 aa, synthetic construct
  REGION 1..116: note = Description of Artificial Sequence: Synthetic polypeptide
EVQLLESGGGLVQPGGSLRLSCAASGFTFSSYAMNWVRQAPGKGLEWVSTTSGSGASTYYADSVKGRFTISRDNSKNTLYLQMNSLRAEDTAVYYCAKIWIAFDIWGQGTMVTVSS

SEQ ID NO: 733 - protein, 108 aa, synthetic construct
  REGION 1..108: note = Description of Artificial Sequence: Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPYTFGQGTKLEIK

SEQ ID NO: 734 - protein, 7 aa, synthetic construct
  REGION 1..7: note = Description of Artificial Sequence: Synthetic peptide
SFNYYWS

SEQ ID NO: 735 - protein, 16 aa, synthetic construct
  REGION 1..16: note = Description of Artificial Sequence: Synthetic peptide
YIYYSGSTYSNPSLKS

SEQ ID NO: 736 - protein, 9 aa, synthetic construct
  REGION 1..9: note = Description of Artificial Sequence: Synthetic peptide
GYNWNYFDY

SEQ ID NO: 737 - protein, 11 aa, synthetic construct
  REGION 1..11: note = Description of Artificial Sequence: Synthetic peptide
RASQSVDNNLV

SEQ ID NO: 738 - protein, 7 aa, synthetic construct
  REGION 1..7: note = Description of Artificial Sequence: Synthetic peptide
GASTRAT

SEQ ID NO: 739 - protein, 10 aa, synthetic construct [sequence omitted]

SEQ ID NO: 740 - protein, 119 aa, synthetic construct [sequence omitted]

SEQ ID NO: 741 - protein, 108 aa, synthetic construct [sequence omitted]

SEQ ID NO: 742 - protein, 5 aa, synthetic construct
  REGION 1..5: note = Description of Artificial Sequence: Synthetic peptide
TYWMH

SEQ ID NO: 743 - protein, 17 aa, synthetic construct
  REGION 1..17: note = Description of Artificial Sequence: Synthetic peptide
EIDPSDSYSNYNQKFKD

SEQ ID NO: 744 - protein, 11 aa, synthetic construct
  REGION 1..11: note = Description of Artificial Sequence: Synthetic peptide
NGGLGPAWFSY

SEQ ID NO: 745 - protein, 11 aa, synthetic construct
  REGION 1..11: note = Description of Artificial Sequence: Synthetic peptide
KASQYVGTAVA

SEQ ID NO: 746 - protein, 7 aa, synthetic construct
  REGION 1..7: note = Description of Artificial Sequence: Synthetic peptide
SASNRYT

SEQ ID NO: 747 - protein, 9 aa, synthetic construct
  REGION 1..9: note = Description of Artificial Sequence: Synthetic peptide
QQYSSYPWT

SEQ ID NO: 748 - protein, 120 aa, synthetic construct
  REGION 1..120: note = Description of Artificial Sequence: Synthetic polypeptide
EVQLVQSGAEVKKPGSSVKVSCKASGYTFTTYWMHWVRQAPGQGLEWIGEIDPSDSYSNYNQKFKDRATLTVDKSTSTAYMELSSLRSEDTAVYYCARNGGLGPAWFSYWGQGTLVTVSS

SEQ ID NO: 749 - protein, 107 aa, synthetic construct
  REGION 1..107: note = Description of Artificial Sequence: Synthetic polypeptide
DIQMTQSPSSVSASVGDRVTITCKASQYVGTAVAWYQQKPGKSPKLLIYSASNRYTGVPSRFSDSGSGTDFTLTISSLQPEDFATYFCQQYSSYPWTFGGGTKVEIK

SEQ ID NO: 750 - protein, 5 aa, synthetic construct
  REGION 1..5: note = Description of Artificial Sequence: Synthetic peptide
SYAMS

SEQ ID NO: 751 - protein, 17 aa, synthetic construct
  REGION 1..17: note = Description of Artificial Sequence: Synthetic peptide
AISGSGTSTYYADSVKG

SEQ ID NO: 752 - protein, 13 aa, synthetic construct
  REGION 1..13: note = Description of Artificial Sequence: Synthetic peptide
VRYNWNHGDWFDP

SEQ ID NO: 753 - protein, 13 aa, synthetic construct
  REGION 1..13: note = Description of Artificial Sequence: Synthetic peptide
SGSSSNIGNNYVS

SEQ ID NO: 754 - protein, 7 aa, synthetic construct
  REGION 1..7: note = Description of Artificial Sequence: Synthetic peptide
ENYNRPA

SEQ ID NO: 755 - protein, 11 aa, synthetic construct
  REGION 1..11: note = Description of Artificial Sequence: Synthetic peptide
SSWDDSLNYWV

SEQ ID NO: 756 - protein, 122 aa, synthetic construct
  REGION 1..122: note = Description of Artificial Sequence: Synthetic polypeptide
EVQLLESGGGLVQPGGSLRLSCAASGFTFSSYAMSWVRQAPGKGLEWVSAISGSGTSTYYADSVKGRFTISRDNSKNTLYLQMNSLRAEDTAVYYCARVRYNWNHGDWFDPWGQGTLVTVSS

SEQ ID NO: 757 - protein, 110 aa, synthetic construct
  REGION 1..110: note = Description of Artificial Sequence: Synthetic polypeptide
QSVLTQPPSASGTPGQRVTISCSGSSSNIGNNYVSWYQQLPGTAPKLLIYENYNRPAGVPDRFSGSKSGTSASLAISGLRSEDEADYYCSSWDDSLNYWVFGGGTKLTVL

SEQ ID NO: 758 - protein, 5 aa, synthetic construct
  REGION 1..5: note = Description of Artificial Sequence: Synthetic peptide
SYGMS

SEQ ID NO: 759 - protein, 17 aa, synthetic construct
  REGION 1..17: note = Description of Artificial Sequence: Synthetic peptide
TISSGGSYKYYVDSVKG

SEQ ID NO: 760 - protein, 11 aa, synthetic construct
  REGION 1..11: note = Description of Artificial Sequence: Synthetic peptide
HPDYDGVWFAY

SEQ ID NO: 761 - protein, 10 aa, synthetic construct
  REGION 1..10: note = Description of Artificial Sequence: Synthetic peptide
SVSSSVFYVH

SEQ ID NO: 762 - protein, 7 aa, synthetic construct
  REGION 1..7: note = Description of Artificial Sequence: Synthetic peptide
DTSKLAS

SEQ ID NO: 763 - protein, 9 aa, synthetic construct
  REGION 1..9: note = Description of Artificial Sequence: Synthetic peptide
QQWNSNPPT

SEQ ID NO: 764 - protein, 120 aa, synthetic construct
  REGION 1..120: note = Description of Artificial Sequence: Synthetic polypeptide
EVQLVESGGGLVQPGGSLRLSCAASGFTFNSYGMSWVRQAPGKGLEWVATISSGGSYKYYVDSVKGRFTISRDNAKNSLYLQMNSLRAEDTAVYYCARHPDYDGVWFAYWGQGTLVTVSS

SEQ ID NO: 765 - protein, 107 aa, synthetic construct
  REGION 1..107: note = Description of Artificial Sequence: Synthetic polypeptide
DIQMTQSPSSLSASVGDRVTITCSVSSSVFYVHWYQQKPGKAPKLLIYDTSKLASSGVPSRFSGSGSGTDFTFTISSLQPEDIATYYCQQWNSNPPTFGGGTKVEIK

SEQ ID NO: 766 - protein, 5 aa, synthetic construct
  REGION 1..5: note = Description of Artificial Sequence: Synthetic peptide
SYGMS

SEQ ID NO: 767 - protein, 17 aa, synthetic construct
  REGION 1..17: note = Description of Artificial Sequence: Synthetic peptide
TISSGGSYTYYPDSVKG

SEQ ID NO: 768 - protein, 11 aa, synthetic construct
  REGION 1..11: note = Description of Artificial Sequence: Synthetic peptide
HPDYDGVWFAY

SEQ ID NO: 769 - protein, 10 aa, synthetic construct
  REGION 1..10: note = Description of Artificial Sequence: Synthetic peptide
SVSSSVFYVH

SEQ ID NO: 770 - protein, 7 aa, synthetic construct
  REGION 1..7: note = Description of Artificial Sequence: Synthetic peptide
DTSKLAS

SEQ ID NO: 771 - protein, 9 aa, synthetic construct
  REGION 1..9: note = Description of Artificial Sequence: Synthetic peptide
QQWNSNPPT

SEQ ID NO: 772 - protein, 120 aa, synthetic construct
  REGION 1..120: note = Description of Artificial Sequence: Synthetic polypeptide
EVQLVESGGDLVKPGGSLKLSCAASGFTFNSYGMSWVRQTPDKRLEWVATISSGGSYTYYPDSVKGRFTISRDNAKNTLYLQMSSLKSEDTAMYYCARHPDYDGVWFAYWGQGTLVTVSA

SEQ ID NO: 773 - protein, 105 aa, synthetic construct
  REGION 1..105: note = Description of Artificial Sequence: Synthetic polypeptide
QIVLTQSPAIMASPGEKVTMTCSVSSSVFYVHWYQQKSGTSPKRWIYDTSKLASGVPARFSGSGSGTSYSLTISSMEAEDAATYYCQQWNSNPPTFGGGTKLEIK

SEQ ID NO: 774 - protein, 5 aa, synthetic construct
  REGION 1..5: note = Description of Artificial Sequence: Synthetic peptide
SYYMH

SEQ ID NO: 775 - protein, 17 aa, synthetic construct
  REGION 1..17: note = Description of Artificial Sequence: Synthetic peptide
IINPSGGSTSYAQKFQG

SEQ ID NO: 776 - protein, 16 aa, synthetic construct
  REGION 1..16: note = Description of Artificial Sequence: Synthetic peptide
DGVLRYFDWLLDYYYY

SEQ ID NO: 777 - protein, 11 aa, synthetic construct
  REGION 1..11: note = Description of Artificial Sequence: Synthetic peptide
RASQSVGSYLA

SEQ ID NO: 778 - protein, 7 aa, synthetic construct
  REGION 1..7: note = Description of Artificial Sequence: Synthetic peptide
DASNRAT

SEQ ID NO: 779 - protein, 8 aa, synthetic construct
  REGION 1..8: note = Description of Artificial Sequence: Synthetic peptide
QQRANVFT

SEQ ID NO: 780 - protein, 128 aa, synthetic construct
  REGION 1..128: note = Description of Artificial Sequence: Synthetic polypeptide
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDGVLRYFDWLLDYYYYMDVWGKGTTVTVSS

SEQ ID NO: 781 - protein, 106 aa, synthetic construct
  REGION 1..106: note = Description of Artificial Sequence: Synthetic polypeptide
EIVLTQSPATLSLSPGERATLSCRASQSVGSYLAWYQQRPGQAPRLLIYDASNRATGIPARFSGSGSGTDFTLTISSLEPEDFAVYYCQQRANVFTFGQGTKVEIK

SEQ ID NO: 782 - protein, 5 aa, synthetic construct
  REGION 1..5: note = Description of Artificial Sequence: Synthetic peptide
SYYMH

SEQ ID NO: 783 - protein, 17 aa, synthetic construct
  REGION 1..17: note = Description of Artificial Sequence: Synthetic peptide
IINPSGGSTSYAQKFQG

SEQ ID NO: 784 - protein, 19 aa, synthetic construct
  REGION 1..19: note = Description of Artificial Sequence: Synthetic peptide
DAELRHFDHLLDYHYYMDV

SEQ ID NO: 785 - protein, 11 aa, synthetic construct
  REGION 1..11: note = Description of Artificial Sequence: Synthetic peptide
RASQSVGSYLA

SEQ ID NO: 786 - protein, 7 aa, synthetic construct
  REGION 1..7: note = Description of Artificial Sequence: Synthetic peptide
DASNRAT

SEQ ID NO: 787 - protein, 8 aa, synthetic construct
  REGION 1..8: note = Description of Artificial Sequence: Synthetic peptide
QQRAQEFT

SEQ ID NO: 788 - protein, 128 aa, synthetic construct
  REGION 1..128: note = Description of Artificial Sequence: Synthetic polypeptide
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDAELRHFDHLLDYHYYMDVWGQGTTVTVSS

SEQ ID NO: 789 - protein, 106 aa, synthetic construct
  REGION 1..106: note = Description of Artificial Sequence: Synthetic polypeptide
EIVMTQSPATLSLSPGERATLSCRASQSVGSYLAWYQQKPGQAPRLLIYDASNRATGIPARFSGSGSGTDFTLTISSLQPEDFAVYYCQQRAQEFTFGQGTKVEIK

SEQ ID NO: 790 - protein, 120 aa, synthetic construct
  REGION 1..120: note = Description of Artificial Sequence: Synthetic polypeptide
QVQLVQSGSELKKPGAPVKVSCKASGYTFSTFGMSWVRQAPGQGLKWMGWIHTYAGVPIYGDDFKGRFVFSLDTSVSTAYLQISSLKAEDTAVYFCARRSDNYRYFFDYWGQGTTVTVSS

SEQ ID NO: 791 - protein, 107 aa, synthetic construct
  REGION 1..107: note = Description of Artificial Sequence: Synthetic polypeptide
DIQMTQSPSSLSASLGDRVTITCRASQDIRNYLNWYQQKPGKAPKLLIYYTSRLHSGVPSRFSGSGSGTDYTLTISSLQPEDFATYFCQQGHTLPPTFGQGTKLEIK

SEQ ID NO: 792 - protein, 120 aa, synthetic construct
  REGION 1..120: note = Description of Artificial Sequence: Synthetic polypeptide
QIQLVQSGPELKKPGAPVKISCKASGYTFTTFGMSWVKQAPGQGLKWMGWIHTYAGVPIYGDDFKGRFVFSLDTSVSTAYLQISSVKAEDTATYFCARRSDNYRYFFDYWGQGTTLTVSS

SEQ ID NO: 793 - protein, 107 aa, synthetic construct; identical to SEQ ID NO: 791
  REGION 1..107: note = Description of Artificial Sequence: Synthetic polypeptide

SEQ ID NO: 794 - protein, 5 aa, synthetic construct
  REGION 1..5: note = Description of Artificial Sequence: Synthetic peptide
NYYMA

SEQ ID NO: 795 - protein, 17 aa, synthetic construct
  REGION 1..17: note = Description of Artificial Sequence: Synthetic peptide
SITKGGGNTYYRDSVKG

SEQ ID NO: 796 - protein, 14 aa, synthetic construct
  REGION 1..14: note = Description of Artificial Sequence: Synthetic peptide
QVTIAAVSTSYFDS

SEQ ID NO: 797 - protein, 15 aa, synthetic construct
  REGION 1..15: note = Description of Artificial Sequence: Synthetic peptide
KTNQKVDYYGNSYVY

SEQ ID NO: 798 - protein, 7 aa, synthetic construct
  REGION 1..7: note = Description of Artificial Sequence: Synthetic peptide
LASNLAS

SEQ ID NO: 799 - protein, 9 aa, synthetic construct
  REGION 1..9: note = Description of Artificial Sequence: Synthetic peptide
QQSRNLPYT

SEQ ID NO: 800 - protein, 123 aa, synthetic construct
  REGION 1..123: note = Description of Artificial Sequence: Synthetic polypeptide
EVQLVESGGGLVQSGRSIRLSCAASGFSFSNYYMAWVRQAPSKGLEWVASITKGGGNTYYRDSVKGRFTFSRDNAKSTLYLQMDSLRSEDTATYYCARQVTIAAVSTSYFDSWGQGVMVTVSS

SEQ ID NO: 801 - protein, 110 aa, synthetic construct
  REGION 1..110: note = Description of Artificial Sequence: Synthetic polypeptide
DIVLTQSPALAVSLGQRATISCKTNQKVDYYGNSYVYWYQQKPGQQPKLLIYLASNLASGIPARFSGRGSGTDFTLTIDPVEADDTATYYCQQSRNLPYTFGAGTKLELK

SEQ ID NO: 802 - protein, 5 aa, synthetic construct
  REGION 1..5: note = Description of Artificial Sequence: Synthetic peptide
DYYMK

SEQ ID NO: 803 - protein, 17 aa, synthetic construct
  REGION 1..17: note = Description of Artificial Sequence: Synthetic peptide
DIIPSNGATFYNQKFKG

SEQ ID NO: 804 - protein, 11 aa, synthetic construct
  REGION 1..11: note = Description of Artificial Sequence: Synthetic peptide
SHLLRASWFAY

SEQ ID NO: 805 - protein, 17 aa, synthetic construct
  REGION 1..17: note = Description of Artificial Sequence: Synthetic peptide
KSSQSLLNSGNQKNYLT

SEQ ID NO: 806 - protein, 7 aa, synthetic construct
  REGION 1..7: note = Description of Artificial Sequence: Synthetic peptide
WASTRES

SEQ ID NO: 807 - protein, 9 aa, synthetic construct
  REGION 1..9: note = Description of Artificial Sequence: Synthetic peptide
QNDYSYPYT

SEQ ID NO: 808 - protein, 120 aa, synthetic construct
  REGION 1..120: note = Description of Artificial Sequence: Synthetic polypeptide
QVQLVQSGAEVKKPGASVKMSCKASGYTFTDYYMKWVKQAPGQGLEWIGDIIPSNGATFYNQKFKGKATLTVDRSISTAYMHLNRLRSDDTAVYYCTRSHLLRASWFAYWGQGTLVTVSS

SEQ ID NO: 809 - protein, 113 aa, synthetic construct
  REGION 1..113: note = Description of Artificial Sequence: Synthetic polypeptide
DFVMTQSPDSLAVSLGERATINCKSSQSLLNSGNQKNYLTWYLQKPGQPPKLLIYWASTRESGVPDRFSGSGSGTDFTLTISSLQAEDVAVYYCQNDYSYPYTFGQGTKLEIK

SEQ ID NO: 810 - protein, 5 aa, synthetic construct
  REGION 1..5: note = Description of Artificial Sequence: Synthetic peptide
DYEMH

SEQ ID NO: 811 - protein, 20 aa, synthetic construct
  REGION 1..20: note = Description of Artificial Sequence: Synthetic peptide
WIGGIDPETGGTAYNQKFKG

SEQ ID NO: 812 - protein, 6 aa, synthetic construct
  REGION 1..6: note = Description of Artificial Sequence: Synthetic peptide
YYSFAY

SEQ ID NO: 813 - protein, 16 aa, synthetic construct
  REGION 1..16: note = Description of Artificial Sequence: Synthetic peptide
RSSQSIVHSNGNTYLQ

SEQ ID NO: 814 - protein, 7 aa, synthetic construct
  REGION 1..7: note = Description of Artificial Sequence: Synthetic peptide
KVSNRFS

SEQ ID NO: 815 - protein, 9 aa, synthetic construct
  REGION 1..9: note = Description of Artificial Sequence: Synthetic peptide
FQVSHVPYT

SEQ ID NO: 816 - protein, 115 aa, synthetic construct
  REGION 1..115: note = Description of Artificial Sequence: Synthetic polypeptide
EVQLVQSGAEVKKPGATVKISCKVSGYTFTDYEMHWVQQAPGKGLEWMGGIDPETGGTAYNQKFKGRVTLTADKSTDTAYMELSSLRSEDTAVYYCGRYYSFAYWGQGTLVTVSS

SEQ ID NO: 817 - protein, 112 aa, synthetic construct
  REGION 1..112: note = Description of Artificial Sequence: Synthetic polypeptide
DVVMTQSPLSLPVTLGQPASISCRSSQSIVHSNANTYLQWFQQRPGQSPRLLIYKVSNRFSGVPDRFSGSGSGTDFTLKISRVEAEDVGVYYCFQVSHVPYTFGQGTKLEIK

SEQ ID NO: 818 - protein, 5 aa, synthetic construct
  REGION 1..5: note = Description of Artificial Sequence: Synthetic peptide
DYNVN

SEQ ID NO: 819 - protein, 17 aa, synthetic construct
  REGION 1..17: note = Description of Artificial Sequence: Synthetic peptide
VINPKYGTTRYNQKFKG

SEQ ID NO: 820 - protein, 7 aa, synthetic construct
  REGION 1..7: note = Description of Artificial Sequence: Synthetic peptide
GLNAWDY

SEQ ID NO: 821 - protein, 11 aa, synthetic construct
  REGION 1..11: note = Description of Artificial Sequence: Synthetic peptide
GASENIYGALN

SEQ ID NO: 822 - protein, 7 aa, synthetic construct
  REGION 1..7: note = Description of Artificial Sequence: Synthetic peptide
GATNLED

SEQ ID NO: 823 - protein, 9 aa, synthetic construct
  REGION 1..9: note = Description of Artificial Sequence: Synthetic peptide
QNVLTTPYT

SEQ ID NO: 824 - protein, 116 aa, synthetic construct; identical to SEQ ID NO: 532
  REGION 1..116: note = Description of Artificial Sequence: Synthetic polypeptide

SEQ ID NO: 825 - protein, 107 aa, synthetic construct; identical to SEQ ID NO: 533
  REGION 1..107: note = Description of Artificial Sequence: Synthetic polypeptide

SEQ ID NO: 826 - protein, 5 aa, synthetic construct
  REGION 1..5: note = Description of Artificial Sequence: Synthetic peptide
GYFMN

SEQ ID NO: 827 - protein, 17 aa, synthetic construct
  REGION 1..17: note = Description of Artificial Sequence: Synthetic peptide
LINPYNGDSFYNQKFKG

SEQ ID NO: 828 - protein, 8 aa, synthetic construct
  REGION 1..8: note = Description of Artificial Sequence: Synthetic peptide
GLRRDFDY

SEQ ID NO: 829 - protein, 16 aa, synthetic construct
  REGION 1..16: note = Description of Artificial Sequence: Synthetic peptide
KSSQSLLDSDGKTYLN

SEQ ID NO: 830 - protein, 7 aa, synthetic construct
  REGION 1..7: note = Description of Artificial Sequence: Synthetic peptide
LVSELDS

SEQ ID NO: 831 - protein, 9 aa, synthetic construct
  REGION 1..9: note = Description of Artificial Sequence: Synthetic peptide
WQGTHFPRT

SEQ ID NO: 832 - protein, 117 aa, synthetic construct; identical to SEQ ID NO: 540
  REGION 1..117: note = Description of Artificial Sequence: Synthetic polypeptide

SEQ ID NO: 833 - protein, 112 aa, synthetic construct; identical to SEQ ID NO: 541
  REGION 1..112: note = Description of Artificial Sequence: Synthetic polypeptide

SEQ ID NO: 834 - protein, 5 aa, synthetic construct
  REGION 1..5: note = Description of Artificial Sequence: Synthetic peptide
TAAIS

SEQ ID NO: 835 - protein, 17 aa, synthetic construct
  REGION 1..17: note = Description of Artificial Sequence: Synthetic peptide
GIIPIFGKAHYAQKFQG

SEQ ID NO: 836 - protein, 14 aa, synthetic construct
  REGION 1..14: note = Description of Artificial Sequence: Synthetic peptide
KFHFVSGSPFGMDV

SEQ ID NO: 837 - protein, 11 aa, synthetic construct
  REGION 1..11: note = Description of Artificial Sequence: Synthetic peptide
RASQSVSSYLA

SEQ ID NO: 838 - protein, 7 aa, synthetic construct
  REGION 1..7: note = Description of Artificial Sequence: Synthetic peptide
DASNRAT

SEQ ID NO: 839 - protein, 8 aa, synthetic construct
  REGION 1..8: note = Description of Artificial Sequence: Synthetic peptide
QQRSNWPT

SEQ ID NO: 840 - protein, 123 aa, synthetic construct; identical to SEQ ID NO: 556
  REGION 1..123: note = Description of Artificial Sequence: Synthetic polypeptide

SEQ ID NO: 841 - protein, 106 aa, synthetic construct; identical to SEQ ID NO: 557
  REGION 1..106: note = Description of Artificial Sequence: Synthetic polypeptide

SEQ ID NO: 842 - protein, 9 aa, synthetic construct
  REGION 1..9: note = Description of Artificial Sequence: Synthetic peptide
GTFSSYAIS

SEQ ID NO: 843 - protein, 17 aa, synthetic construct
  REGION 1..17: note = Description of Artificial Sequence: Synthetic peptide
SIIPIFGTANYAQKFQG

SEQ ID NO: 844 - protein, 18 aa, synthetic construct
  REGION 1..18: note = Description of Artificial Sequence: Synthetic peptide
ARGPSEVGAILGYVWFDP

SEQ ID NO: 845 - protein, 16 aa, synthetic construct
  REGION 1..16: note = Description of Artificial Sequence: Synthetic peptide
RSSQSLLHSNGYNYLD

SEQ ID NO: 846 - protein, 7 aa, synthetic construct
  REGION 1..7: note = Description of Artificial Sequence: Synthetic peptide
LGSNRAS

SEQ ID NO: 847 - protein, 9 aa, synthetic construct
  REGION 1..9: note = Description of Artificial Sequence: Synthetic peptide
MQARRIPIT

SEQ ID NO: 848 - protein, 125 aa, synthetic construct
  REGION 1..125: note = Description of Artificial Sequence: Synthetic polypeptide
QVQLVQSGAEVKKPGSSVKVSCKASGGTFSSYAISWVRQAPGQGLEWMGSIIPIFGTANYAQKFQGRVTITADESTSTAYMELSSLRSEDTAVYYCARGPSEVGAILGYVWFDPWGQGTLVTVSS

SEQ ID NO: 849 - protein, 112 aa, synthetic construct
  REGION 1..112: note = Description of Artificial Sequence: Synthetic polypeptide
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQARRIPITFGGGTKVEIK

SEQ ID NO: 850 - protein, 12 aa, synthetic construct
  REGION 1..12: note = Description of Artificial Sequence: Synthetic peptide
GYTFTDHAIHWV

SEQ ID NO: 851 - protein, 10 aa, synthetic construct
  REGION 1..10: note = Description of Artificial Sequence: Synthetic peptide
FSPGNDDIKY

SEQ ID NO: 852 - protein, 8 aa, synthetic construct
  REGION 1..8: note = Description of Artificial Sequence: Synthetic peptide
KRSLSTPY

SEQ ID NO: 853 - protein, 12 aa, synthetic construct
  REGION 1..12: note = Description of Artificial Sequence: Synthetic peptide
QSLLNRGNHKNY

SEQ ID NO: 854 - protein, 7 aa, synthetic construct
  REGION 1..7: note = Description of Artificial Sequence: Synthetic peptide
WASTRES

SEQ ID NO: 855 - protein, 9 aa, synthetic construct
  REGION 1..9: note = Description of Artificial Sequence: Synthetic peptide
QNDYTYPYT

SEQ ID NO: 856 - protein, 115 aa, synthetic construct; identical to SEQ ID NO: 620
  REGION 1..115: note = Description of Artificial Sequence: Synthetic polypeptide

SEQ ID NO: 857 - protein, 113 aa, synthetic construct; identical to SEQ ID NO: 621
  REGION 1..113: note = Description of Artificial Sequence: Synthetic polypeptide

SEQ ID NO: 858 - protein, 5 aa, synthetic construct
  REGION 1..5: note = Description of Artificial Sequence: Synthetic peptide
NYDIN

SEQ ID NO: 859 - protein, 17 aa, synthetic construct
  REGION 1..17: note = Description of Artificial Sequence: Synthetic peptide
WIYPGDGSTKYNEKFKA

SEQ ID NO: 860 - protein, 8 aa, synthetic construct
  REGION 1..8: note = Description of Artificial Sequence: Synthetic peptide
GYEDAMDY

SEQ ID NO: 861 - protein, 11 aa, synthetic construct
  REGION 1..11: note = Description of Artificial Sequence: Synthetic peptide
KASQDINSYLS

SEQ ID NO: 862 - protein, 7 aa, synthetic construct
  REGION 1..7: note = Description of Artificial Sequence: Synthetic peptide
RANRLVD

SEQ ID NO: 863 - protein, 9 aa, synthetic construct
  REGION 1..9: note = Description of Artificial Sequence: Synthetic peptide
LQYDEFPLT

SEQ ID NO: 864 - protein, 117 aa, synthetic construct
  REGION 1..117: note = Description of Artificial Sequence: Synthetic polypeptide
QVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSS

SEQ ID NO: 865 - protein, 105 aa, synthetic construct
  REGION 1..105: note = Description of Artificial Sequence: Synthetic polypeptide
DIQMTQSPSSLSASVGDRVTINCKASQDINSYLSWFQQKPGKAPKTLIYRANRLVDGVPSRFSGSGSGQDYTLTISSLQPEDFATYYCLQYDEFPLTFGGGTKVE

SEQ ID NO: 866 - protein, 5 aa, synthetic construct
  REGION 1..5: note = Description of Artificial Sequence: Synthetic peptide
NYGMN

SEQ ID NO: 867 - protein, 17 aa, synthetic construct
  REGION 1..17: note = Description of Artificial Sequence: Synthetic peptide
WINTYSGEPRYADDFKG

SEQ ID NO: 868 - protein, 9 aa, synthetic construct
  REGION 1..9: note = Description of Artificial Sequence: Synthetic peptide
DYGRWYFDV

SEQ ID NO: 869 - protein, 10 aa, synthetic construct
  REGION 1..10: note = Description of Artificial Sequence: Synthetic peptide
RASSSVSHMH

SEQ ID NO: 870 - protein, 7 aa, synthetic construct
  REGION 1..7: note = Description of Artificial Sequence: Synthetic peptide
ATSNLAS

SEQ ID NO: 871 - protein, 9 aa, synthetic construct
  REGION 1..9: note = Description of Artificial Sequence: Synthetic peptide
QQWSSTPRT

SEQ ID NO: 872 - protein, 118 aa, synthetic construct; identical to SEQ ID NO: 158
  REGION 1..118: note = Description of Artificial Sequence: Synthetic polypeptide

SEQ ID NO: 873 - protein, 106 aa, synthetic construct
  REGION 1..106: note = Description of Artificial Sequence: Synthetic polypeptide
QIVLSQSPATLSLSPGERATMSCRASSSVSHMHWYQQKPGQAPRPWIYATSNLASGVPARFSGSGSGTDYTLTISSLEPEDFAVYYCQQWSSTPRTFGGGTKVEIK

SEQ ID NO: 874 - protein, 5 aa, synthetic construct
  REGION 1..5: note = Description of Artificial Sequence: Synthetic peptide
DYYIH

SEQ ID NO: 875 - protein, 17 aa, synthetic construct
  REGION 1..17: note = Description of Artificial Sequence: Synthetic peptide
YINPNSGYTNYAQKFQG

SEQ ID NO: 876 - protein, 12 aa, synthetic construct
  REGION 1..12: note = Description of Artificial Sequence: Synthetic peptide
YMWERVTGFFDF

SEQ ID NO: 877 - protein, 11 aa, synthetic construct
  REGION 1..11: note = Description of Artificial Sequence: Synthetic peptide
LASEDISDDLA

SEQ ID NO: 878 - protein, 7 aa, synthetic construct
  REGION 1..7: note = Description of Artificial Sequence: Synthetic peptide
TTSSLQS

SEQ ID NO: 879 - protein, 9 aa, synthetic construct
  REGION 1..9: note = Description of Artificial Sequence: Synthetic peptide
QQTYKFPPT

SEQ ID NO: 880 - protein, 121 aa, synthetic construct
  REGION 1..121: note = Description of Artificial Sequence: Synthetic polypeptide
QVQLVQSGAEVKKPGASVKLSCKASGYTFTDYYIHWVRQAPGQGLEWIGYINPNSGYTNYAQKFQGRATMTADKSINTAYVELSRLRSDDTAVYFCTRYMWERVTGFFDFWGQGTMVTVSS

SEQ ID NO: 881 - protein, 107 aa, synthetic construct
  REGION 1..107: note = Description of Artificial Sequence: Synthetic polypeptide
DIQMTQSPSSVSASVGDRVTITCLASEDISDDLAWYQQKPGKAPKVLVYTTSSLQSGVPSRFSGSGSGTDFTLTISSLQPEDFATYFCQQTYKFPPTFGGGTKVEIK

SEQ ID NO: 882 - protein, 8 aa, synthetic construct
  REGION 1..8: note = Description of Artificial Sequence: Synthetic peptide
GFTFSNYA

SEQ ID NO: 883 - protein, 8 aa, synthetic construct
  REGION 1..8: note = Description of Artificial Sequence: Synthetic peptide
ISGSGDYT

SEQ ID NO: 884 - protein, 11 aa, synthetic construct
  REGION 1..11: note = Description of Artificial Sequence: Synthetic peptide
ARSPWGYYLDS

SEQ ID NO: 885 - protein, 6 aa, synthetic construct
  REGION 1..6: note = Description of Artificial Sequence: Synthetic peptide
QGISSR

SEQ ID NO: 886 - skipped

SEQ ID NO: 887 - protein, 9 aa, synthetic construct
  REGION 1..9: note = Description of Artificial Sequence: Synthetic peptide
QQYNSYPYT

SEQ ID NO: 888 - protein, 118 aa, synthetic construct; identical to SEQ ID NO: 612
  REGION 1..118: note = Description of Artificial Sequence: Synthetic polypeptide

SEQ ID NO: 889 - protein, 107 aa, synthetic construct; identical to SEQ ID NO: 613
  REGION 1..107: note = Description of Artificial Sequence: Synthetic polypeptide

SEQ ID NO: 890 - protein, 444 aa, synthetic construct
  REGION 1..444: note = Description of Artificial Sequence: Synthetic polypeptide
  REGION 1..444: note = hS2C6 heavy chain
EVQLVESGGGLVQPGGSLRLSCAASGYSFTGYYIHWVRQAPGKGLEWVARVIPNAGGTSYNQKFKGRFTLSVDNSKNTAYLQMNSLRAEDTAVYYCAREGIYWWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 891 - protein, 219 aa, synthetic construct
  REGION 1..219: note = Description of Artificial Sequence: Synthetic polypeptide
  REGION 1..219: note = hS2C6 light chain
DIQMTQSPSSLSASVGDRVTITCRSSQSLVHSNGNTFLHWYQQKPGKAPKLLIYTVSNRFSGVPSRFSGSGSGTDFTLTISSLQPEDFATYFCSQTTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 892 - protein, 440 aa, synthetic construct
  REGION 1..440: note = Description of Artificial Sequence: Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 893 - protein, 214 aa, synthetic construct
  REGION 1..214: note = Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 894 - protein, 447 aa, synthetic construct
  REGION 1..447: note = Description of Artificial Sequence: Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 895 - protein, 218 aa, synthetic construct
  REGION 1..218: note = Description of Artificial Sequence: Synthetic polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 896 - protein, 451 aa, synthetic construct
  REGION 1..451: note = Description of Artificial Sequence: Synthetic polypeptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 897 - protein, 215 aa, synthetic construct
  REGION 1..215: note = Description of Artificial Sequence: Synthetic polypeptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 898 - protein, 448 aa, synthetic construct
  REGION 1..448: note = Description of Artificial Sequence: Synthetic polypeptide
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 899 - protein, 214 aa, synthetic construct
  REGION 1..214: note = Description of Artificial Sequence: Synthetic polypeptide
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC